SEQUENCE LISTING (6 sequences)

SEQ ID NO: 1 - protein, 122 aa, Streptococcus pneumoniae
  mat_peptide (1)..()
MRMIVGHGIDIEELASIESAVTRHEGFAKRVLTAQEMERFTSLKGRRQIEYLAGRWSAKEAFSKAMGTGISKLGFQDLEVLNNERGAPYFSQAPFSGKIWLSISHTDQFVTASVILEENHES

SEQ ID NO: 2 - DNA, 34 bp, Artificial Sequence
  PCR Primer
cgcggatcccatatgatagttggacacggaattg

SEQ ID NO: 3 - DNA, 31 bp, Artificial Sequence
  PCR Primer
cgcggatccctagctttcatgaatttcctcc

SEQ ID NO: 4 - DNA, 43 bp, Artificial Sequence
  PCR Primer
cgcggatcccatatgacagaaaaagaaatttttgaccgtattg

SEQ ID NO: 5 - DNA, 45 bp, Artificial Sequence
  PCR Primer
cgcggatccgaattcctattttccttgaatgattttaaccacatc

SEQ ID NO: 6 - protein, 9 aa, Streptococcus pneumoniae
MIVGHGIDI

(19) United States
(12) Patent Application Publication
(10) Pub. No.: US 2003/0068802 A1
(43) Pub. Date: Apr. 10, 2003

(54) Use of streptococcus pneumoniae acyl carrier protein synthase crystal structure in diagnostics, antimicrobial drug design, and biosensors

(76) Inventors: Nicholas Yuri Chirgadze, Indianapolis, IN (US); Stephen Lyle Briggs, Indianapolis, IN (US); Genshi Zhao, Indianapolis, IN (US); Kelly Ann McAllister, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 09/897,645

(22) Filed: Jun. 29, 2001

Related U.S. Application Data
(60) Provisional application No. 60/215,577, filed on Jun. 30, 2000.

(51) Int. Cl. C12N 9/10; G06F 19/00; G01N 33/48; G01N 33/50

(52) U.S. Cl. 435/193; 702/19

(57) ABSTRACT

Provided are methods of purifying and crystallizing Streptococcus pneumoniae acyl carrier protein synthase (AcpS) enzyme, crystals of AcpS, the use of such crystals to determine the three-dimensional structure of AcpS enzymes, and the three-dimensional structure of AcpS. The three-dimensional crystal structure of AcpS can be used in medical diagnostics to produce antibodies that permit detection of Streptococcus pneumoniae both in vitro and in vivo. The three-dimensional crystal structure of AcpS can also be used in pharmaceutical discovery and development to identify and design compounds that inhibit the biochemical activity of AcpS enzyme in bacteria. Inhibitory compounds identified in this way can be optimized by structure/activity studies to develop antibacterial pharmaceutical compounds useful for the prevention or treatment of bacterial infections.

This application claims the benefit of priority of U.S. Provisional Patent Application Serial No. 60/215,577 filed on Jun. 30, 2000, the contents of which are herein incorporated by reference in their entirety.

### BACKGROUND OF THE INVENTION

1. Field of the Invention

The present invention relates to the fields of protein crystallography, medical diagnostics, and pharmaceutical development. More particularly, the present invention relates to methods of purifying and crystallizing Streptococcus pneumoniae acyl carrier protein synthase (AcpS) enzyme, crystals of AcpS enzyme, the use of these crystals to determine the three-dimensional structure of AcpS enzyme, and to the three-dimensional structure of AcpS enzyme. The three-dimensional crystal structure of the AcpS enzyme can be used in medical diagnostics to produce antibodies that permit detection of Streptococcus pneumoniae both in vitro and in vivo, and therefore accurate diagnosis of infections caused by this bacterium. The three-dimensional crystal structure of AcpS can also be used in pharmaceutical discovery and development to identify and design compounds that inhibit the biochemical activity of AcpS enzyme in bacteria. The inhibitory activity of compounds identified in this way can be optimized by structure/activity studies to develop antibacterial pharmaceutical compounds for the prevention or treatment of bacterial infections in mammals.

2. Description of Related Art

Bacterial Drug Resistance

The emerging resistance of bacteria to antibiotics is a frightening clinical problem (Cohen, 1992; Neu, 1992; Davies, 1994; Spratt, 1994a). A number of common pathogenic bacterial species such as Streptococcus pneumoniae, Staphylococcus aureus, Enterococcus, Shigella dysenteriae, and Mycobacterium tuberculosis have developed resistance to almost all of the antibiotics, including β-lactams and quinolones, two of the largest and most important classes of antibiotics that have been widely prescribed for upper respiratory tract infections (Cohen, 1992; Neu, 1992; Davies, 1994; Spratt, 1994b; Tomasz and Munoz, 1995; Thomson and Sanders, 1998; Ahamed et al., 1999). The β-lactam and quinolone antibiotics are known to target the biosynthesis of bacterial cell walls and DNA replication, respectively (Neu, 1992; Davies, 1994; Spratt, 1994b; Tomasz and Munoz, 1995 Thomson and Sanders, 1998).

Bacterial Acyl Carrier Protein Synthases

The biosynthesis of fatty acids is known to be required for the growth of bacteria as fatty acids are essential components of bacterial membrane lipids and lipopoly-saccharides (Cronan and Rock, 1996; Rock and Cronan, 1996). The fatty acid biosynthetic pathway in bacteria is well characterized (Cronan and Rock, 1996; Rock and Cronan, 1996). Bacteria utilize the type II, or dissociated fatty acid synthase system, for fatty acid synthesis (Cronan and Rock, 1996; Rock and Cronan, 1996). The type II fatty acid synthase system consists of individual enzymes that are encoded by separate genes (Cronan and Rock, 1996; Rock and Cronan, 1996). On the other hand, the type I fatty acid synthase system, almost exclusively present in eukaryotes, is characterized by the presence of a multifunctional protein that possesses all the catalytic activities required for fatty acid synthesis (S. Smith, 1994). In both systems, fatty acids are synthesized by using a repeated cycle of condensation, reduction, dehydration, and reduction reactions (Cronan and Rock, 1996; Rock and Cronan, 1996; S. Smith, 1994). In these reactions, holo-acyl carrier protein (holo-ACP) plays an essential role as an acyl carrier for fatty acid precursors, growing acyl intermediates, and nascent fatty acid products (Cronan and Rock, 1996; Rock and Cronan, 1996; S. Smith, 1994; Elovson and Vagelos, 1968; Lambalot and Walsh, 1995).

ACP is a small acidic protein in bacteria (Cronan and Rock, 1996; Rock and Cronan, 1996) or a small domain of the type I fatty acid synthase in eukaryotes (S. Smith, 1994). ACP in E. coli is encoded by the acpP gene (Cronan and Rock, 1996; Rock and Cronan, 1996). The newly synthesized ACP, or apo-ACP, is not functional in fatty acid synthesis. The conversion of apo-ACP to holo-ACP, by ACP synthase (AcpS) is required for its functionality (Cronan and Rock, 1996; Rock and Cronan, 1996; Elovson and Vagelos, 1968; Lambalot and Walsh, 1995). The enzymatic step of the reaction that converts apo-acyl carrier protein (apo-ACP) to holo-ACP (Elovson and Vagelos, 1968; Lam et al., 1992; Lambalot and Walsh, 1995; Lambalot et al., 1996; McAllister et al., 2000) is catalyzed by ACP synthase (AcpS), encoded by the acpS gene. This reaction involves the transfer of the 4′-phosphopantetheine group of Coenzyme A (CoA) onto a serine residue of apo-ACP, thereby converting apo-ACP to holo-ACP (Elovson and Vagelos, 1968; Lam et al., 1992; Lambalot and Walsh, 1995; Lambalot et al., 1996). The resulting holo-ACP mediates the transfer of fatty acid intermediates during the biosynthesis of fatty acids and lipids via the covalent attachment of carboxyl groups of fatty acid intermediates to the thiol of the 4′-phosphopantetheine prosthetic group of holo-ACP (Elovson and Vagelos, 1968; Lam et al., 1992; Magnuson et al., 1993; Lambalot and Walsh, 1995; Lambalot et al., 1996). This reaction is therefore required for the biosynthesis of all bacterial fatty acids, lipid A, which is an essential component of bacterial lipopolysacchrides, and the membrane lipids, which are also derived exclusively from acyl intermediates of fatty acids (Magnuson et al., 1993). The essential nature of this reaction has been well established genetically in E. coli and S. pneumoniae (Lam et al., 1992; Takiff et al., 1992; McAllister et al., 2000). Consistent with its important role in fatty acid biosynthesis, AcpS is widely present in Mycoplasma, as well as gram-negative and gram-positive bacteria. Homologues of AcpS and ACP have been identified in many bacterial genomes sequenced to date (Blattner et al., 1997; Cole et al., 1998; Himmelreich et al., 1996; Kalman et al., 1999); Kuns et al., 1997; Tomb et al., 1997). Thus, AcpS appears to be an attractive antibacterial target for discovery of novel antimicrobial agents.

E. coli AcpS has been well studied (Majerus et al., 1965; Elovson and Vagelos, 1968; Lambalot and Walsh, 1995; Lambalot et al., 1996; Gehring et al., 1997; Flugel et al., 2000). The acpS gene from E. coli forms an operon with the upstream gene, pdxJ, whose function is required for vitamin B₆ biosynthesis (Lam et al., 1992; Takiff et al., 1992). The acpS gene was originally identified as dpj (downstream of pdxJ) whose function, although unknown, was required for the growth of E. coli (Lam et al., 1992; Takiff et al., 1992). Later, the landmark biochemical study by Lambalot and Walsh (1995) led to the identification of Dpj as AcpS. E. coli AcpS is a small, highly basic protein of approximately 14 kDa (Lambalot and Walsh, 1995). The E. coli enzyme has been purified and characterized (Lambalot and Walsh, 1995). The enzyme exhibits a broad substrate specificity, and can utilize a variety of AcpS, which are required for many diverse aspects of cellular metabolism (Majerus et al., 1965; Crosby et al., 1995; Lambalot and Walsh, 1995; Lambalot et al., 1996; Carreas et al., 1997; Gehring et al., 1997; Tropf et al., 1998; Kutchma et al., 1999; Zhou et al, 1999; Flugel et al., 2000). Purified AcpS also exhibits activity with a number of CoA derivatives (Gehring et al., 1997). These results indicate that AcpS may be able to participate in other metabolism besides fatty acid biosynthesis in the cell. Purified AcpS also exhibits activity with a number of CoA derivatives (Gehring et al., 1997). Finally, AcpS is a very low abundance protein in E. coli (Elovson and Vagelos, 1968; Lamabolt and Walsh, 1995). In contrast, ACP is a very abundant protein that has been estimated to be present at 25,000-60,000 molecules per cell (Cronan and Rock, 1996; Rock and Cronan, 1996; Jackowski and Rock, 1984; Vallari and Jackowshi, 1988). The majority of AcpS present in the cell are found to be holo-AcpS (Cronan and Rock, 1996; Rock and Cronan, 1996; Jackowski and Rock, 1983; Heath and Rock, 1996).

Although E. coli AcpS is well studied, the reaction mechanism of AcpS remains unknown. In addition, only the AcpS from E. coli, a rod-shaped, Gram-negative bacterium, has been thoroughly characterized to date. It still remains to be determined whether AcpS from Gram-positive bacteria plays the same physiological role. Finally, AcpS appears to possess all the features necessary for a good antibacterial target, such as its essential nature, widespread existence in bacteria, and unique catalytic position in an important biosynthetic pathway (fatty acid biosynthesis). Thus, AcpS appears to be a valuable antibacterial target for identifying novel antimicrobial agents.

Recently, AcpS from Streptococcus pneumoniae has been purified and characterized (McAllister et al., 2000). The S. pneumoniae enzyme exhibits biochemical properties similar to those of the E. coli AcpS. The acpS gene has also been shown to be essential for the growth of S. pneumoniae (McAllister et al., 2000).

The crystal structure of Bacillus subtilis AcpS has recently been described by Parris et al. (2000).

The pressing need for new antibacterial compounds can desirably be met by methods that do not rely on serendipity and/or systematic screening of large numbers of natural and synthetic compounds. One such method relies on structure-based drug design using computer modeling and the three-dimensional crystal structure of bacterial target proteins to identify and optimize antimicrobial drug candidates. The three-dimensional structure of AcpS has heretofore remained unknown as no crystals thereof have been produced that permitted the required crystallographic data to be obtained. Therefore, there is a need in the art for such crystals, for the determination of the three-dimensional structure of such crystals, and for structure-based drug design and diagnostics based on such crystallographic data.

### SUMMARY OF THE INVENTION

Accordingly, in order to exploit bacterial cellular components that can serve as diagnostic agents and antibacterial targets for identifying novel antibiotics that can be used to combat the current crisis of antibiotic resistance, the present inventors have focused their attention on AcpS. The present invention includes among its various aspects crystals of AcpS enzyme, use of such AcpS crystals to determine the three-dimensional structure of AcpS enzyme, the three-dimensional crystal structure of AcpS enzyme, methods of drug discovery/design and diagnostics based on the three-dimensional crystal structure AcpS enzyme, use of crystals of AcpS in biosensors and other applications, and methods of purifying and crystallizing AcpS enzyme. As a first step toward structure-based drug design, the present invention provides the crystal structures of S. pneumoniae AcpS and the AcpS/3′,5′-ADP complex at 2.0 and 1.9 Å resolution, respectively.

More specifically, in a first aspect, the present invention provides a composition comprising a crystal of isolated Streptococcus pneumoniae AcpS. Such crystal effectively diffracts X-rays, and permits the determination of the atomic coordinates of the AcpS to a resolution of 2.0 Å or greater. As used herein, the term “greater” refers to resolution of the atomic coordinates to a value lower than 2.0 Å.

More particularly, the present invention provides a composition comprising a crystal of Streptococcus pneumoniae AcpS having the amino acid sequence shown in SEQ ID NO: 1 wherein methionine is substituted with selenomethioinine, wherein said AcpS is a homotrimer, wherein each protomer comprises the following structural motifs: (a) a three-stranded anti-parallel β-sheet formed by strands β1, β5, and β4; (b) a long α-helix that packs diagonally against said β-sheet, together with α-helices α1 α2, α3, and α4 of an anti-parallel four helical bundle; and (c) a long, extended loop with a two-strand anti-parallel β-sheet comprising strands β2 and β3, wherein said structural motifs (a), (b), and (c) are organized such that said long helix α4 runs through said homotrimer, and is surrounded by the remainder of said structural motifs, as shown in FIGS. 8(B) and 8(C). When the AcpS is in native form, i.e., the active site is unoccupied, the crystal belongs to orthorhombic space group P2₁2₁2₁, with unit cell dimensions of a=49.8 Å, b=59.6 Å, and c=114.7 Å, or monoclinic space group C2, with unit cell dimensions of a=120.2 Å, b=62.3 Å, c=51.7 Å, and β=98.7°. Such crystals can have the atomic coordinates shown in Tables 3 and 4. When the AcpS is complexed with 3′,5′-adenosine diphosphate, the crystal belongs to monoclinic space group C2, with unit cell dimensions of a=120.2 Å, b=62.3 Å, c=51.7 Å, and β=98.7°, and the crystal can have the atomic coordinates shown in Table 5.

In another aspect, the present invention provides an enzyme active site crystal structure comprising the 3′,5′-adenosine diphosphate binding site shown in FIG. 9. More particularly, the enzyme comprises isolated, properly folded Streptococcus pneumoniae AcpS, or a fragment or protein fusion product thereof comprising the active site. Also encompassed by the present invention are active site crystal structures that are variants or homologs of the AcpS binding pocket having a root mean square deviation from the amino acid residues comprising the AcpS active site shown in FIG. 9 in the range of from about 1 to about 3 Å, more preferably from about 1 to about 2 Å, and most preferably of about 1.15 Å. Such active sites can form binding complexes with an ACP, a CoA, an apo-ACP, an acetyl-CoA, a desulfo-CoA, an acetoacetyl-CoA, a malonyl-CoA, or a dephospho-CoA.

In a further aspect, the present invention provides a method of isolating Streptococcus pneumoniae AcpS, comprising:

(a) growing Streptococcus pneumoniae in a medium lacking methionine but containing L-selenomethionine;

(b) preparing a cell extract of the Streptococcus pneumoniae;

(c) centrifuging the cell extract to produce a supernatant fraction, and collecting this supernatant fraction;

(d) chromatographing the supernatant fraction on a cation exchange column in buffer containing dithiothreitol or β-mercaptoethanol, and collecting fractions containing the Streptococcus pneumoniae AcpS; and

(e) chromatographing the fractions of step (d) on a gel filtration column in buffer containing dithiothreitol or β-mercaptoethanol, and collecting fractions containing the Streptococcus pneumoniae AcpS comprising L-selenomethionine.

An additional step in this method can comprise chromatographing the fractions of step (e) on an anion exchange column in buffer containing dithiothreitol or β-mercapto-ethanol, and collecting fractions containing Streptococcus pneumoniae AcpS.

In yet another aspect, the present invention provides isolated Streptococcus pneumoniae acyl carrier protein synthase produced by the foregoing method.

In another aspect, the present invention provides a method of producing a crystal of Streptococcus pneumoniae AcpS that diffracts X-rays, comprising:

(a) providing Streptococcus pneumoniae AcpS isolated according to the foregoing method;

(b) concentrating the AcpS to 8 mg/ml in a solution containing 10 mM MgCl₂, 14 mM KCl, and 20 mM Tris-HCl at pH 7.1;

(c) equilibrating a 4 μl drop of the AcpS in a solution comprising a mixture of 1:1, v/v, protein solution/reservoir solution over a 500 μl reservoir solution comprising 8-15% polyethyleneglycol 4000, 200 mM ammonium sulfate, and 100 mM citrate buffer at pH 4.5; and

(d) growing a crystal of AcpS by vapor diffusion at 294K for 4 to 5 days or longer.

In another aspect, the present invention provides a crystal of Streptococcus pneumoniae AcpS produced by the foregoing method.

In a further aspect, the present invention provides a co-crystal of Streptococcus pneumoniae AcpS with a compound, produced by either including such compound in the protein solution/reservoir solution during crystallization, or by contacting a crystal of AcpS and a solution, such as protein or reservoir solution, comprising such compound (“soaking” the crystal in a solution of compound), and incubating the resulting mixture to permit the compound to diffuse into the crystal, thereby producing a complex between AcpS and the compound. The compound can be 3′,5′-adenosine diphosphate. Whatever compound is employed, a three-dimensional structure of the resulting acyl carrier protein synthase/compound crystal complex can then be determined.

In another aspect, the present invention provides a co-crystal of AcpS produced by the foregoing or similar method.

In yet other aspects, the present invention provides the use of a three-dimensional crystal or co-crystal structure of AcpS in medical diagnostics, biosensors, and pharmaceutical drug discovery and design. The latter enables methods of treating Streptococcal infections utilizing compounds that block the biochemical activity of Streptococcus pneumoniae AcpS enzyme, by, for example, determining a three-dimensional structure of AcpS enzyme from Streptococcus pneumoniae, utilizing such three-dimensional structure to identify/develop a compound that binds to and inhibits the enzyme, and contacting, either in vitro or in vivo, such compound and Streptococcal cells containing the enzyme, thereby inhibiting the biochemical activity of the enzyme and treating or preventing Streptococcal infections.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### BRIEF DESCRIPTION OF THE DRAWINGS AND TABLES

The file of this patent contains at least one drawing and one table executed in color. Copies of this patent with color drawing(s) and tables will be provided by the Patent and Trademark Office upon request and payment of the necessary fee.

The above and other aspects, features, and advantages of the present invention will be better understood from the following detailed description taken in conjunction with the accompanying drawings, all of which are given by way of illustration only, and are not limitative of the present invention, in which:

FIGS. 1A and 1B show SDS-PAGE analysis of purified S. pneumoniae AcpS and apo-ACP, respectively, expressed in E. coli, and purified as described in Example 1. The purified AcpS is analyzed by SDS-PAGE (16% Tricine gels) and stained with Coomassie Blue R-250. FIG. 1A: Each lane contains 5 μg of protein. Lane M: prestained molecular weight markers. Lane 1: Crude extract of E. coli containing overexpressed S. pneumoniae AcpS. Lane 2: pooled fractions from 15S Source S column; Lane 3: pooled fractions from S-100 Sepharose column. FIG. 1B: Each lane contains 10 μg of protein. Lane M: prestained molecular weight markers. Lane 1: Crude extract of E. coli containing overexpressed S. pneumoniae ACP. Lane 2: pooled fractions from Source 15Q column; Lane 3: pooled fractions from S-100 Sepharose column.

FIG. 2 shows analysis of the native structure of S. pneumoniae AcpS and apo-ACP by gel filtration column chromatography. Both AcpS and apo-ACP are purified as described in Example 1. Purified AcpS (27 μM) and apo-ACP (100 μM) are subjected to gel filtration column (S-75 Sephadex) chromatography, and their molecular weights are determined as described in Example 1. Panel A shows gel filtration column chromatograph. Peak A contains both AcpS and apo-ACP, and has an elution volume of 10.5 ml with an estimated molecular weight of 53 kDa. Peak B contains only AcpS, and has an elution volume of 11.5 ml, with an estimated molecular weight of 38 kDa. The arrow C points to the area where apo-ACP elutes. Since apo-ACP does not absorb at 280 nm, there is no apparent protein peak observed. Panel B shows SDS-PAGE analysis of the column fractions. All relevant fractions collected from Panel A are subjected to SDS-PAGE analysis. The gels are stained with SYPRO Orange, and analyzed using a FluorImager.

FIG. 3 shows analysis of the S. pneumoniae AcpS and apo-ACP native structures by cross-linking. AcpS and apo-ACP are purified as described in Example 1. Purified AcpS (163 μM) and apo-ACP (94 μM) are treated without or with 19.5 and 9.4 mM sulfo-EGS, respectively. The resulting AcpS (Panel A) and apo-ACP (Panel B) preparations are analyzed by SDS-PAGE (16% tricine gels). Panel A: Lane 1: the pre-stained molecular weight marker; lane 2: AcpS untreated with sulfo-EGS; and lane 3: AcpS treated with sulfo-EGS. Panel B: Lane 1, the pre-stained molecular weight marker; lane 2: apo-ACP untreated with sulfo-EGS; lane 3: apo-ACP treated with sulfo-EGS.

FIG. 4 shows a kinetic analysis of the effect of apo-ACP concentrations on the AcpS activity of S. pneumoniae. AcpS activity is measured using the HPLC method under conditions where the CoA concentration is fixed at 20 μM and the apo-ACP concentration is varied from 0.5 to 100 μM. A: the substrate (apo-ACP) saturation curve of AcpS. B and C: the double reciprocal plots of the initial velocities of the enzyme versus the various apo-ACP concentrations (<5 μM and >15 μM, respectively).

FIG. 5 shows a kinetic analysis of the effect of CoA concentrations on the AcpS activity of S. pneumoniae. AcpS activity is measured by the HPLC method under conditions where the CoA concentrations are varied (2.5-600 μM) and the apo-ACP concentration is fixed (2 μM). A: the substrate (CoA) saturation curve of AcpS. B: the double reciprocal plot of the initial velocities of AcpS versus CoA concentrations.

FIG. 6 shows an analysis of the mechanism of the AcpS catalyzed reaction.

AcpS activity is measured using HPLC methods. A: double-reciprocal plots of the initial velocities of AcpS versus the various CoA concentrations (2.5 -30 μM) and the fixed apo-ACP concentrations (0.25 (●),0.35 (▪), 0.5 (▴), and 0.6 μM (♦)). B: double-reciprocal plots of the initial velocities of AcpS versus the various apo-ACP concentrations (0.25-0.6 μM) and the fixed CoA concentrations (2.5 (●) , 5.0 (▪), 10 (▴), and 20 μM (♦)).

FIG. 7 shows an analysis of the inhibition kinetics of 3′,5′-ADP with respect to apo-ACP and CoA. AcpS activity is measured in the absence (●) or presence of 3′,5′-ADP (5 (▪) and 10 μM (▴)). A and B: the double-reciprocal plots of the initial 2 5 velocities of AcpS versus the various CoA concentrations and the fixed apo-ACP concentration, and the various apo-ACP concentrations and the fixed CoA concentrations, respectively. C and D: Dixon plots of A and B, respectively. The CoA concentrations used in panel C are 10 (●) and 40 (▪) μM.

FIG. 8A is a color stereoview showing a ribbon diagram of the AcpS homotrimer, viewed along a non-crystallographic 3-fold axis.

FIG. 8B is a color ribbon diagram of the Cα backbone of one Streptococcus pneumoniae AcpS monomer structure.

FIG. 8C is a color topology (Richardson) diagram of AcpS. β-strands are represented as arrows, while α helices are rectangles. The secondary structure elements are defined as follows: β1, Ile4-Glu13; α1, Leu14-Arg23; α2, Phe27-Val31; α3, Ala34-Ser42; α4, Gly45-Met66; α5, Ile70-Leu73; β2, Glu79-Asn82; β3, Pro88-Gln92; β4, Lys98-His105; β5, Phe109-Glu117.

FIG. 9 is a color stereoview of the 3′,5′-ADP fragment of CoA bound to the active site of S. pneumoniae AcpS. The omitted electron density map corresponding to the ligand is contoured at the 1 σ level at 1.9 Å resolution.

FIG. 10 shows a color superposition of apo-AcpS from S. pneumoniae (shown in blue) on the surfactin synthetase activating enzyme Sfp (4′-phosphopantetheinyl transferase) from Bacillus subtilis complexed with CoA (shown in red). One protomer of AcpS is superimposed on the N-terminal domain of Sfp, and a second AcpS protomer is superimposed on the C-terminal domain of Sfp. The third protomer of the AcpS trimer does not have a counterpart in the Sfp structure. A sulfate ion is found in the AcpS binding site that corresponds to the position of the α-phosphate of CoA in the Sfp molecule.

Table 1A shows sequence alignment of bacterial AcpS genes from different species. The most conserved regions are shown in grey. Secondary structural elements observed in the S. pneumoniae AcpS crystal structure are indicated above the sequence pile-up.

Table 1B shows structural alignment of two Streptococcus pneumoniae AcpS monomer molecules with two domains of Bacillus subtilis Sfp. The N-terminal half of Sfp from Metl to ProlO3 corresponds to one protomer of the AcpS trimer (shown in blue), and has a 22% sequence identity. The C-terminal half of Sfp from Ile104 to Pro209 corresponds to a second AcpS protomer (shown in green) and has 25% sequence identity. The remaining C-terminal portion from Asp210 to Leu224 has no counterpart in the AcpS structure. The three amino acid residues involved in Mg²⁺ binding are marked by an asterisk (*). The regions involved in CoA binding are marked by plus (+) signs.

Table 2 (1 page) summarizes the crystallographic data disclosed herein.

Table 3 (51 pages) shows the atomic coordinates for AcpS native 1.

Table 4 (52 pages) shows the atomic coordinates for AcpS native 2.

Table 5 (53 pages) shows the atomic coordinates for AcpS/3′,5′-ADP complex.

The tables herein are presented after the Abstract of the Disclosure.

### DETAILED DESCRIPTION OF THE INVENTION

The following detailed description of the invention is provided to aid those skilled in the in art in practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

The present invention provides novel Streptococcus pneumoniae AcpS crystal structures, including their active sites, use of these crystal structures to produce antibodies to epitopes of this protein for in vitro and in vivo diagnostic purposes, and methods of using these crystal structures and active sites to identify or design AcpS inhibitor compounds for use as antibacterials in the treatment of bacterial infections. Among its many aspects, the present invention provides a method for inhibiting AcpS by administering compounds having certain structural, physical, and spatial characteristics that permit interaction, e.g., binding, of such compounds with specific amino acid residues within acyl carrier protein synthases, particularly the active sites of these enzymes. Such compounds may bind to all or only a portion of an active site, and may be competitive or non-competitive inhibitors of these enzymes.

As used herein, the term “crystal” includes an ordered protein array having a regular structure of a constituent chemical species, e.g., such as an AcpS protein or portion thereof. In one embodiment, a crystal of the present invention comprises a solid three-dimensional AcpS protein aggregate in which planar surfaces intersect at definite angles. In another embodiment, a crystal of the invention comprises an ordered two dimensional packing of AcpS proteins, such as, e.g., in a monolayer.

As used herein, the term “active site” refers to the general region of an enzyme molecule containing the catalytic residues identified with the binding of substrate(s) (and prosthetic group(s), if any), and reaction of substrate(s) by the making and breaking of bonds. “Catalytic residues” include any of the amino acid residues in an enzyme that are directly involved in making or breaking covalent bonds while the enzyme is acting on a substrate. It includes those amino acid residues that are, in the enzyme-substrate complex, either contact amino acids, i.e., those that at some point are within only one bond distance of some point on the substrate molecule (which may include both catalytic residues and specificity residues), or auxiliary amino acids, i.e., those that are not in such intimate physical contact with the substrate, but which nonetheless play a definite role in the action of the enzyme. The active site generally takes up a relatively small part of the total volume of an enzyme molecule. Most of the amino acids residues in an enzyme are not in contact with the substrate. The active site is a three-dimensional entity, often a cleft or crevice, formed by groups that come from different parts of the linear amino acid sequence. Residues far apart in the linear sequence may interact more strongly than adjacent residues in the amino acid sequence. Substrates are bound to enzymes by multiple weak attractions, including electrostatic bonds, hydrogen bonds, van der Waals forces, and hydrophobic interactions. The enzyme and substrate should have complementary shapes. Finally, the specificity of binding depends on the precisely defined arrangement of atoms in the active site, and the shapes of the active sites of some enzymes are markedly modified by the binding of substrate. Thus, the active sites of these enzymes have shapes that are complementary to that of the substrate only after the substrate is bound, a process of dynamic recognition called “induced fit.”

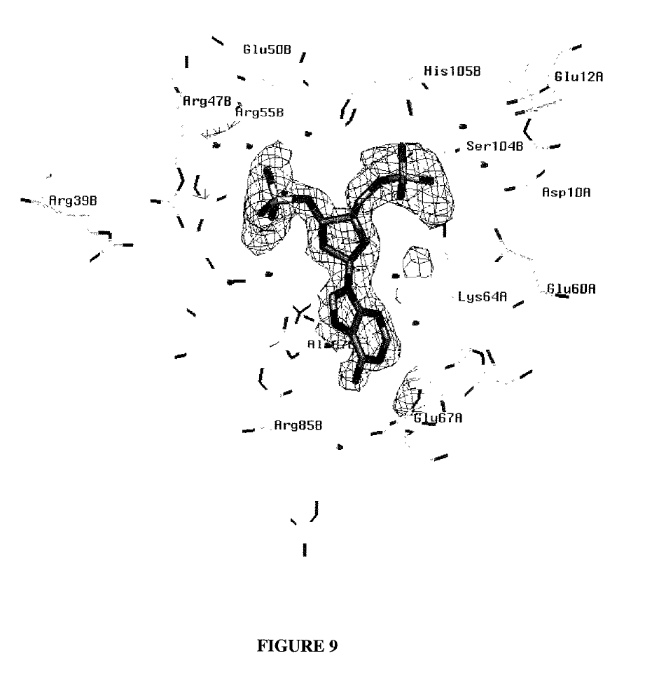

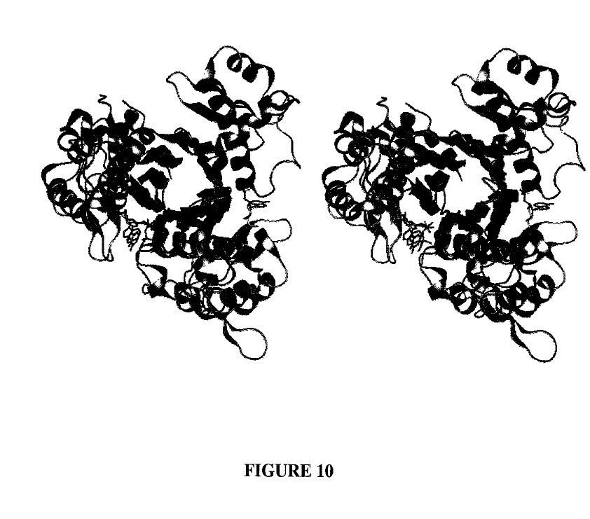

Inhibitors of acyl carrier protein synthases are predicted to interact with (bind) these proteins in the region comprising the 3′,5′-adenosine diphosphate binding site, shown in FIG. 9.

As used herein, including the appended claims, singular forms of words such as “a,” “an,” and “the” include, e.g., their corresponding plural referents unless the context clearly dictates otherwise. Thus, for example, reference to “a crystal” includes, e.g., one or more crystals, reference to “an AcpS crystal” includes, e.g., one or more of such crystals; reference to a binding event includes one or more such events; reference to bind a crystal includes binding one or more such crystals; and reference to “a method” includes, e.g., reference to equivalent steps and methods known to a person of ordinary skill in the art, and so forth.

With respect to antibodies for diagnostic purposes, the crystal structures disclosed herein facilitate the production of such antibodies. Knowledge of the three-dimensional structure of AcpS provides information as to various epitopes on the enzyme surface. Using this knowledge, one can synthesize peptides comprising such epitopes, and produce antibodies that specifically bind thereto by methods well known in the art. Antibodies produced in response to these peptides can then be used to detect the presence of Streptococcus pneumoniae both in vitro and in vivo, thereby providing useful diagnostic means for detecting the presence of this pathogen.

Structure-Based Drug Design

The use of protein crystal structures to design candidate antimicrobial compounds can be accomplished by structure-based drug design. In one embodiment of this method, the three-dimensional structure of a protein or protein fragment, such as a peptide, is determined, and potential antagonists (or agonists, if desired) are designed with the aid of computer modeling (Bugg et al.,1993; West et al., 1995). Once the crystal structure of the target, e.g., Streptococcus pneumoniae AcpS is determined, computer modeling is conducted (using programs such as DOCK or Multiple Copy Simultaneous Search (MCSS)) to construct candidate inhibitor compounds based on the crystal structure. These compounds are chemically synthesized, or obtained from previously existing drug libraries, and their binding to the AcpS target and/or their biological activity, i.e., inhibitory activity, against AcpS is assayed. Compounds that bind and/or that inhibit the enzymatic activity of AcpS are thereby identified as drug candidates. In a further refinement of this method, those compounds exhibiting binding and/or activity can be associated or complexed with the crystal for further X-ray diffraction analysis to map their interactions with the crystal structure. This can be accomplished by growing a supplemental crystal of AcpS in the presence of one such compound to produce a crystal containing a complex formed between the AcpS protein and the drug candidate. A crystal is chosen that effectively diffracts X-rays, allowing the determination of the atomic coordinates of the protein-ligand complex to a resolution greater than about 10 Å, preferably to a resolution greater than about 5 Å, more preferably to a resolution greater than about 3 Å, more preferably to a resolution greater than about 2.0 Å, and most preferably to a resolution of about 1.9 Å. The three-dimensional structure of the supplemental crystal is determined by molecular replacement analysis, and a drug is selected by performing rational drug design with the three-dimensional structure determined using the supplemental crystal. Such selecting is preferably performed in conjunction with computer modeling.

From the resulting inhibitor-target crystal structure, one of ordinary skill in the art can construct further improved candidate compounds.

The steps set forth in the preceding paragraphs can be repeated and refined as desired until a drug candidate having the desired potency is identified.

Protein Expression

As would be apparent to those of ordinary skill in the art, conventional molecular biological, microbiological, and recombinant DNA techniques are available that permit expression of amounts of AcpS protein sufficient for the types of studies described herein using genomic DNA, cDNA, synthetic DNA, etc. coding on expression therefor. Such techniques are fully explained in the literature, e.g., Sambrook et al., 1989 and Ausubel et al., 1987. Note also in this regard U.S. Pat. Nos. 6,020,162 and 6,087,478.

Preparation of Protein Crystals

Crystals of AcpS can be grown by a number of techniques, including batch crystallization, vapor diffusion (either by sitting drop, hanging drop, or sandwich), microdialysis, membrane crystallization, or any other conventional method of protein crystallization. Seeding of crystals is sometimes required in order to obtain X-ray quality crystals. Standard micro- and/or macro-seeding of crystals can therefore be employed. As described below, the hanging drop method can be used to obtain the crystals disclosed herein.

X-ray Diffraction

Once a crystal of the present invention is grown, X-ray diffraction data can be collected. Crystals can be characterized, for example, by using X-rays produced in a conventional source (such as a sealed tube or a rotating anode), or using a synchrotron source. Methods of characterization include, but are not limited to, precision photography, oscillation photography, and diffractometer data collection. Heavy atom derivatives, such as produced using Hg, Pb, Au, U, Pt, I, Os, etc., can be performed using Fuji imaging plates. Alternatively, AcpS can be synthesized with selenium-methionine (Se-Met) in place of methionine, and the Se-Met multiwavelength anomalous dispersion data (Hendrickson, 1991) can be collected on CHESS F2, using reverse-beam geometry to record Friedel pairs at four X-ray wavelengths, corresponding to two remote points above and below the Se absorption edge (λ₁ and λ₄) and the absorption edge inflection point (λ₂) and peak (λ₃). Selenium sites can be located using SHELXS-90 in Patterson search mode (G. M. Sheldrick). Experimental phases (α_{MAD}) can be estimated via a multiple isomorphous replacement/anomalous scattering strategy using MLPHARE (Z. Otwinowski, Southwestern University of Texas, Dallas) with three of the wavelengths treated as derivatives and one (λ₂) treated as the parent for example. In either case, data can be processed using HKL, DENZO, and SCALEPACK (Otwinowski and Minor, 1997).

In addition, X-PLOR (Brünger, 1992; X-PLOR v. 3.1 Manual, New Haven, Yale University) or Heavy (T. Terwilliger, Los Alamos National Laboratory) can be utilized for bulk solvent correction and B-factor scaling. After density modification and non-crystallographic averaging, the protein is built into an electron density map using the program O (Jones et al., 1991). Model building interspersed with positional and simulated annealing refinement (Brünger, 1992, supra) can permit the unambiguous trace and sequence assignment of the AcpS protein.

Protein-Structure Based Design of Agonists and Antagonists of AcpS

Once the three-dimensional structure of a crystal comprising the AcpS protein, or fragment or fusion protein derivative thereof comprising an active site, is determined, a potential ligand (antagonist or agonist) can be examined via the use of computer modeling using a docking program such as RAM, DOCK, or AUTODOCK (Dunbrack et al., 1997). This procedure can include computer fitting of potential ligands to AcpS to ascertain how well the shape and the chemical structure of the potential ligand will complement or interfere with the enzyme (Bugg et al., 1993; West et al., 1995). Computer programs can also be employed to estimate the attraction, repulsion, and steric hindrance of the ligand to the AcpS binding site. Generally the tighter the fit (e.g., the lower the steric hindrance, and/or the greater the attractive force) the more potent the potential drug will be since these properties are consistent with a tighter binding constant. Furthermore, the greater the specificity in the design of a potential drug, the more likely that the drug will not interfere with other properties of the AcpS protein or other proteins in host cells. This will minimize potential side-effects due to unwanted interactions with other proteins.

Initially, a potential ligand could be obtained by screening, for example, a random peptide library produced by recombinant bacteriophage (Scott et al., 1990; Cwirla et al., 1990; Devlin et al., 1990) or a chemical library. A ligand selected in this manner could be then be systematically modified by computer modeling programs until one or more promising potential ligands are identified. Such analysis has been shown to be effective in the development of HIV protease inhibitors (Lam et al., 1994; Wlodawer et al., 1993; Appelt, 1993; Erickson, 1993).

Such computer modeling allows the selection of a finite number of rational chemical modifications, as opposed to the countless number of essentially random chemical modifications that could be made, and of which any one might lead to a useful drug. Each chemical modification requires additional chemical steps, which while being reasonable for the synthesis of a finite number of compounds, quickly becomes overwhelming if all possible modifications needed to be synthesized. Thus, through the use of the three-dimensional structure disclosed herein in combination with computer modeling, a large number of these compounds can be rapidly screened on the computer monitor screen, and a few likely candidates can be determined without the laborious synthesis of untold numbers of compounds.

Once a potential ligand (agonist or antagonist) is identified, it can either be selected from a library of chemicals as are commercially available from most large chemical companies including Eli Lilly and Company, or alternatively, the potential ligand can be synthesized de novo. As noted above, the de novo synthesis of one or even a relatively small group of specific compounds is reasonable in the art of drug design. The prospective drug can be placed into any standard binding assay described below to test its effect on the AcpS protein.

When a suitable drug is identified, a supplemental crystal can be grown which comprises a protein-ligand complex formed between the AcpS protein and the drug.

Preferably, the crystal effectively diffracts X-rays, allowing the determination of the atomic coordinates of the protein-ligand complex to a resolution of greater than about 5.0 Å, more preferably greater than about 3.0 Å, and even more preferably greater than about 2.0 Å. The three-dimensional structure of the supplemental crystal can be determined by molecular replacement analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used for this purpose include X-PLOR and AMORE (J. Navaza, 1994). Once the position and orientation are known, an electron density map can be calculated using the search model to provide X-ray phases. Thereafter, the electron density is inspected for structural differences, and the search model is modified to conform to the new structure. Using this approach, it will be possible to use the claimed structure of the AcpS protein to solve the three-dimensional structures of any such AcpS protein. Other computer programs that can be used to solve the structures of such AcpS crystals include QUANTA, CHARMM. INSIGHT, SYBYL, MACROMODE, and ICM.

For all of the drug screening assays described herein, further refinements to the structure of the drug will generally be necessary and can be made by the successive iterations of any and/or all of the steps provided by the particular drug screening assay.

Binding and Other Assays for Drug Screening

Once identified by computer modeling techniques, candidate compounds can be tested for biological activity using standard techniques. For example, such compounds can be used in assays to assess inhibition of AcpS enzymatic activity, or binding assays using conventional formats to screen inhibitors. Examples of such assays include enzyme-linked immunosorbent assays (ELISA) or fluorescence quench assays. Such compounds can also be tested in in vitro assays designed to assess growth inhibition or killing of the microorganism harboring such enzymes, or in vivo in infected hosts.

It should be noted that the present invention encompasses the use of analogs and derivatives that have the same or substantially similar enzymatic activity as that of the Streptococcus pneumoniae AcpS protein exemplified herein. Such derivatives and analogs include, but are not limited to, AcpS proteins containing amino acid additions, substitutions, or deletions that result in proteins functionally equivalent to the Streptococcus pneumoniae AcpS protein disclosed herein.

AcpS Peptide, Polvpeptide, and Protein Variants

The present invention encompasses AcpS having the amino acid sequence shown in SEQ ID NO: 1, as well as fragments of this protein containing an active site, fusions of AcpS with other proteins, fusions of fragments of this protein containing an active site, as well as amino acid variants of any of these sequences that retain AcpS enzymatic activity.

The peptides, polypeptides, and proteins of the present invention, or variants thereof, can comprise any number of contiguous amino acid residues. The subsequence of contiguous amino acids derived from the protein sequence disclosed herein can be at least about 20, at least about 30, at least about 40, at least about 50, at least about 60, at least about 70, at least about 80, or at least about 90 amino acids in length. Furthermore, the number of contiguous amino acid residues in such subsequences can be any integer selected from the group consisting of from 1 to 20, such as 2, 3, 4, or 5.

In a further aspect, the present invention encompasses an isolated AcpS peptide, polypeptide, or protein comprising an amino acid sequence having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, and even yet more preferably at least about 99% sequence identity, to the corresponding region of the AcpS protein of the present invention (SEQ ID NO: 1).

In a further aspect, the present invention relates to an isolated peptide, polypeptide, or protein comprising an amino acid sequence scoring at least about 80% positives, preferably at least about 81% positives, more preferably at least about 82% positives, yet more preferably at least about 83% positive, yet more preferably at least about 84% positives, yet more preferably at least about 85% positives, yet more preferably at least about 86% positives, yet more preferably at least about 87% positives, yet more preferably at least about 88% positives, yet morepreferably at least about 89% positives, yet more preferably at least about 90% positives, yet more preferably at least about 91% positives, yet more preferably at least about 92% positives, yet more preferably at least about 93% positives, yet more preferably at least about 94% positives, yet more preferably at least about 95% positives, yet more preferably at least about 96% positives, yet more preferably at least about 97% positives, yet more preferably at least about 98% positives, yet more preferably at least about 99% positives, when compared with the amino acid sequence of corresponding residues shown in SEQ ID NO:1.

The present invention encompasses biochemically active variants of the AcpS protein disclosed herein. Biochemical activity includes, for example, AcpS enzymatic activity. Such biochemically active peptides, polypeptides, and proteins have activity that is at least about 20%, 30%, or 40%, preferably at least about 50%, 60%, or 70%, and most preferably at least about 80%, 90%, or 95%-100% of that of the corresponding native (non-synthetic) AcpS protein. Furthermore, the ligand binding specificity of such variant AcpS molecules is substantially similar to that of the corresponding native (non-synthetic) protein. Typically, the ligand binding specificity will be at least about 30%, 40%, or 50% that of the corresponding native (non-synthetic) protein, and more preferably at least about 60%, 70%, 80%, or 90%-100% thereof. Methods of assaying and quantifying measures of biochemical activity and ligand binding by AcpS are described herein; others are well known to those of skill in the art.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

In addition to using D-amino acids, those of ordinary skill in the art are aware that modifications in the amino acid sequence of a peptide, polypeptide, or protein can result in equivalent, or possibly improved, second generation peptides, etc., that display equivalent or superior functional characteristics when compared to the original amino acid sequences. Alterations in the AcpS peptides, polypeptides, or proteins of the present invention can include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) as the naturally occurring counterpart sequences disclosed herein.

One factor that can be considered in making such changes is the hydropathic index of amino acids. The importance of the hydropathic amino acid index in conferring interactive biological function on a protein has been discussed by Kyte and Doolittle (1982). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the no acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in the AcpS molecules of the present invention can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

Particularly preferred conserved amino acid substitutions are:

(a) Lys for His or for Arg, or vice versa, such that a positive charge is maintained;

(b) Glu for Asp, or vice versa, such that a negative charge is maintained;

(c) Ser for Thr, or vice versa, such that a free —OH group is maintained;

(d) Gln for Asn, or vice versa, such that a free —NH₂ group is maintained;

(e) Ile for Leu or for Val, or vice versa, as roughly equivalent hydrophobic amino acids; and

(f) Phe for Tyr, or vice versa, as roughly equivalent aromatic amino acids.

Non-conservative amino acid substitutions can also be introduced if they do not substantially affect either the ligand binding or enzymatic properties of the AcpS protein. Such non-conservative amino acid substitutions can occur in regions of the protein not involved in drug candidate binding, but can also be tolerated in regions involved in such binding if they do not significantly affect such binding, i.e., binding of the ligand occurs substantially the same as in the wild-type Streptococcus pneumoniae AcpS protein. It should be noted that changes that are not expected to be advantageous can also be useful if these result in the production of functional sequences. Since small peptides, etc., can be easily produced by conventional solid phase synthetic techniques, the present invention includes peptides, etc., such as those discussed herein, containing the amino acid modifications discussed above, alone or in various combinations. To the extent that such modifications can be made while substantially retaining the activity of the peptide, etc., they are included within the scope of the present invention. The utility of such modified peptides, etc., can be determined without undue experimentation by, for example, the methods described herein.

While biologically functional equivalents of the present AcpS molecules can have any number of conservative or non-conservative amino acid changes that do not significantly affect their activity(ies), or that increase or decrease activity as desired, 40, 30, 20, 10, 5, or 3 changes, such as 1-30 changes or any range or individual value therein, may be preferred. In particular, 10 or fewer amino acid changes may be preferred. More preferably, seven or fewer amino acid changes may be preferred; more preferably, five or fewer amino acid changes may be preferred; most preferably, three or fewer amino acid changes may be preferred. The encoding nucleotide sequences (gene, plasmid DNA, cDNA, synthetic DNA, or mRNA, for example) will thus have corresponding base substitutions, permitting them to code on expression for the biologically functional equivalent forms of the AcpS molecules. In any case, the AcpS peptides, polypeptides, or proteins exhibit the same or similar biological or immunological activity(ies) as that(those) of the AcpS molecule specifically dislcosed herein, or increased or reduced activity, if desired.

The activity(ies) of the variant AcpS molecules can be determined by the methods described herein or as are known in the art. Variant AcpS molecules biologically functionally equivalent to those specifically disclosed herein have activity(ies) differing from those of the presently disclosed molecules by about ±50% or less, preferably by about ±40% or less, more preferably by about ±30% or less, more preferably by about ±20% or less, more preferably by about ±10% or less, and even more preferably by about ±5% or less, when assayed by the methods disclosed herein, or as are known in the art.

Amino acids in an AcpS molecule of the present invention that are essential for activity can be identified by methods known in the art, such as site-directed mutagenesis or alanine-scanning mutagenesis (Cunningham and Wells, 1989). The latter procedure introduces single alanine mutations at every residue in the molecule. The resulting mutant molecules are then tested for biological activity. Sites that are critical for ligand binding can also be identified by structural analysis such as crystallization, nuclear magnetic resonance, or photoaffinity labeling (Smith et al., 1992, and de Vos et al., 1992).

Methods of Drug Discovery/Design Using the Three-Dimensional Structure of Crystallized AcpS

The three-dimensional crystal structures and atomic coordinates disclosed herein can be used to model an AcpS. These atomic coordinates can be used to computationally design a chemical compound that binds to the active site of an AcpS, and inhibit the enzymatic activity thereof. Such chemical compounds can be used to treat or prevent a Streptococcus pneumoniae infection in a mammal in need thereof.

Numerous methods can be employed to discover/design compounds that bind to AcpS, inhibit its activity, and that can therefore exhibit effective antibacterial activity. It should be noted that in all the methods described herein, one can employ the atomic coordinates of the entire AcpS protein, or just those of an active site.

For example, one method of drug design comprises employing the structural coordinates of a crystal of Streptococcus pneumoniae AcpS to computationally evaluate the ability of a chemical compound to interact with, e.g., bind to an active site of, the AcpS. Such chemical compound can be a competitive, non-competitive, uncompetitive, or mixed inhibitor that binds to, or inhibits the enzymatic activity of, AcpS. For example, the compound can be a competitive inhibitor that binds to the catalytic active site of AcpS.

Another method comprises employing the structural coordinates of a crystal of Streptococcus pneumoniae AcpS to identify an intermediate in a biochemical reaction between AcpS and a compound that is a substrate or inhibitor of this enzyme.

Another method of designing a candidate compound that binds to or inhibits Streptococcus pneumoniae AcpS comprises:

(a) providing the three-dimensional structure of a crystal of Streptococcus pneumoniae AcpS defined by the atomic coordinates shown in Tables 3 and 4, wherein the atomic coordinates include the active site of the synthase; and

(b) designing a candidate compound based upon the three-dimensional crystal structure of the active site of the enzyme.

Another method of designing a compound useful for inhibiting Streptococcus pneumoniae AcpS comprises:

(a) obtaining a crystal of Streptococcus penumoniae AcpS;

(b) evaluating the three-dimensional structure of the crystal;

(c) synthesizing or obtaining from a pre-existing chemical or computer library a potential inhibitor compound based on the three-dimensional crystal structure of the crystal;

(d) contacting the Streptococcus pneumoniae AcpS and the potential inhibitor compound; and

e) assaying the Streptococcus pneumoniae AcpS for activity, wherein a decrease in activity of the AcpS in the presence of the compound compared to the activity of the AcpS in the absence of the compound identifies the compound as an inhibitor of Streptococcus pneumoniae AcpS.

Another method of identifying a compound that binds to and inhibits the enzymatic activity of Streptococcus pneumoniae AcpS or any other AcpS comprising the same or similar active site coordinates (active site configuration) as those of Streptococcus pneumoniae AcpS comprises:

(a) introducing into a suitable computer program, e.g., a docking program, an algorithm for structure-based ligand design/optimization, etc., information defining the conformation of the catalytic active site of Streptococcus pneumoniae AcpS, wherein the program displays the three-dimensional structure of the catalytic active site;

(b) creating a three-dimensional representation of the active site cavity of the Streptococcus pneumoniae AcpS in the computer program;

(c) displaying and superimposing a model of the compound on the three-dimensional representation of the active site cavity of the AcpS;

(d) assessing whether the compound model fits spatially into the active site;

(e) incorporating the compound in a biological or biochemical activity assay for an AcpS comprising the active site; and

(f) determining whether the compound inhibits AcpS activity in the assay.

In another method, the coordinates in Tables 3 and 4 can be used to identify the active site of AcpS, and the obtained information can then be used for visual analysis/inspection of enzyme/inhibitor interactions to identify a compound that inhibits the function of AcpS.

Another method of identifying an inhibitor of AcpS activity comprises docking a computer representation of a first compound structure with a computer representation of the structure of the cavity formed by the active site of an AcpS, thereby forming a complex between the first compound and the AcpS. The method can further comprise:

(a) removing the computer representation of the first compound from the active site, and docking a computer representation of a second compound selected from a computer data base with the computer representation of the active site, thereby forming a second complex comprising the second compound and the AcpS active site;

(b) determining a conformation of the second complex of step (a) with a favorable geometric fit and favorable complementary interactions; and

(c) identifying a compound that best fits the active site as a potential modulator of the AcpS activity.

This method can further comprise:

(a) modifying the computer representation of the first compound by deleting one or more chemical groups from the first compound, or by adding one or more chemical groups to the first compound, thereby forming a second complex;

(b) determining a conformation of the second complex of step (a) with a favorable geometric fit and favorable complementary interactions; and

(c) identifying a compound that best fits said active site as a potential modulator of the AcpS activity.

This method can further comprise:

(a) removing the computer representation of the first compound complexed with the AcpS active site; and

(b) searching a database for a second compound structurally similar to the first compound using a compound searching computer program, or replacing portions of the first compound with chemical structures from a database using a compound construction computer program.

Furthermore, structural information on the interaction of a compound with AcpS can be used to place another compound in the active site, or to perform a virtual screen of a computer library of compounds to identify a lead compound by structure-based drug design.

The foregoing methods can further comprise:

(a) incorporating the second compound in a biological or biochemical activity assay for an AcpS comprising the active site; and

(b) determining whether the second compound inhibits the AcpS enzymatic activity in the assay.

Another method of identifying an inhibitor that competitively binds to the active site of Streptococcus pneumoniae AcpS or other acyl carrier protein synthase having the same or similar active site atomic coordinates comprises:

(a) providing the atomic coordinates of the active site to a computerized modeling system;

(b) identifying a compound that binds to the active site; and

(c) screening the compound identified in step (b) for AcpS inhibitory activity.

Another method of the present invention encompasses solving a crystal structure by using the structural coordinates of Streptococcus pneumoniae AcpS, or a portion thereof, as disclosed herein to solve a crystal form of a mutant, homologue, or co-complex of this AcpS by molecular rearrangement.

Yet other methods of the present invention relate to determining the three-dimensional structure of AcpS enzymes of unknown structure by using information derived from the three-dimensional structure of AcpS as disclosed herein. One such method comprises:

(a) aligning a computer representation of the amino acid sequence of Streptococcus pneumoniae AcpS with a computer representation of the amino acid sequence of another acyl carrier protein synthase by matching homologous regions of amino acid sequences of these representations;

(b) transferring computer representations of the amino acid sequence in said other acyl carrier protein synthase to computer representations of corresponding amino acid sequences in the three-dimensional structure of Streptococcus pneumoniae AcpS; and

(c) determining a low energy conformation of the other acyl carrier protein synthase structure resulting from step (b).

Another such method of determining the three-dimensional structure of an acyl carrier protein synthase of unknown structure comprises:

(a) determining the secondary structure of Streptococcus pneumoniae AcpS having the structural atomic coordinates set forth in Tables 3 and 4 using NMR data; and

(b) simplifying the assignment of through-space interactions of amino acids.

Another method encompassed by the present invention is one for designing a ligand that binds to the active site domain of AcpS. Such method can be computer-based, and can comprise:

(a) providing a model of the crystal structure of the active site domain of Streptococcus pneumoniae AcpS;

(b) analyzing the model to design a ligand that binds to the active site domain; and

(c) determining the effect of the ligand on the active site.

Such an effect might be a conformational change in the active site, covalent bond formation, etc. This method can further comprise modifying the ligand to improve the binding affinity to AcpS, selectivity to AcpS, or both, of the ligand.

Also encompassed by the present invention are antibacterial compounds that modulate AcpS activity, by, for example, binding to and/or inhibiting AcpS, discovered or designed by any of the foregoing methods. Non-limiting examples of such compounds include a compound selected from a computer database, a compound constructed from chemical groups selected from a computer database, a chemically synthesized compound, a naturally occurring compound, a peptide, peptidomimetic, or a natural product molecule that binds in the active site of Streptococcus pneumoniae AcpS.

Such compounds can be used in methods of preventing or treating a Streptococcus pneumoniae infection, or other infection caused by a microorganism having an AcpS enzyme, by administering to a patient in need thereof a pharmaceutically effective amount of such compound.

In addition to the foregoing applications of AcpS crystals, the present invention also encompasses the use of such crystals for a variety of other purposes, such as those discussed below.

AcpS Crystalline Compositions

Enzymes can be employed in a variety functions, such as, for example, as catalysts in large and/or laboratory scale economical production of fine and specialty chemicals (Jones, J. B., Tetrahedron 42: 3351-3403 (1986)), and as tools for the synthesis of organic compounds (Wong, C. H., Science 244: 1145-1152 (1989); Chemtracts-Org. Chem. 3: 91-111 (1990); Klibanov, A. M., Ace. Chem. Res. 23: 114-120 (1990)).

In the clinical area, enzymes can be used in extracorporeal therapy, such as hemodialysis and hemofiltration, where the enzymes selectively remove waste and toxic materials (Klein, M. and Langer, R., Trends in Biotechnology 4: 179-185 (1986)). Enzymes are used in these areas because they function efficiently as catalysts for a broad range of reaction types, at modest temperatures, with substrate specificity, and with stereoselectivity. Nonetheless, there are disadvantages associated with the use of soluble enzyme catalysts that limit their use in industrial and laboratory chemical processes (Akiyama et. al., Chemtech 627-634 (1988)).

Enzymes are expensive and relatively unstable compared to most industrial and laboratory catalysts, even when used in aqueous media where they normally function. Many of the more economically interesting chemical reactions carried out in common practice are incompatible with aqueous media, where, for example, substrates and products are often insoluble or unstable, and where hydrolysis can compete significantly. In addition, the recovery of soluble enzyme catalyst from product and unreacted substrate often requires the application of complicated and expensive separation technology. Finally, enzymes are difficult to store in a manner that retains their activity and functional integrity, for commercially reasonable periods of time (months to years) without resorting to refrigeration (4° C. to −80° C. to liquid N₂ temperatures), or to maintenance in aqueous solvents of suitable ionic strength, pH, etc. The use of crystallized enzymatic proteins can avoid such limitations and is beneficial in medical, clinical, research, and industrial applications. For instance, the slow dissolution rate of protein crystals has been utilized to achieve sustained release of medications, such as crystalline formulations of insulin, interferon-alpha, and pancreatic enzymes. (see, e.g., Matsuda, et al. 1989 J. Biol. Chem. 264, 13381; Peseta, et al., in Annual Review of Biochemistry, Vol. 56, edited by Richardson, et al.; Annual Reviews: Palo Alto, 1989; p. 727; Brange, J. in The Galenics of Insulin. Berlin: Springer, 1987; Reichert, et al., Metal-interferon-alpha crystals. U.S. Pat. No. 5,441,734, 1995; Long, et al., Crystal Growth 1996, 168, 233; and reference to the Cross-Linked Enzyme Crystal formulations (CLEC®) of Altus Biologics Inc. USA).

Accordingly, the present invention encompasses crystal compositions of AcpS.

Such crystals can be produced as described herein or made according to any method in the art such as, e.g., those described in McPherson, et al. 2000 Annu Rev Biophys Biomol Struct.;29:361-410; McPherson, A 1998. Crystallization of Biological Macromolecules, Cold Spring Harbor Laboratory Press, Cold Spring Harbor, N.Y.; Gilliland, G. L. 1988. A biological macromolecule crystallization database: a basis for a crystallization strategy. J. Cryst. Growth 90: 51-59; Chernov A. A. 1998 Acta Crystallogr A. Nov 1;54(Pt 6 Pt 1):859-872; McPherson 1985 Methods Enzymol. 114:112; and Gilliland 1988 J. Crystal Growth 90: 51-59, which include a comprehensive list of suitable conditions in reviews of the crystallization literature. Such an AcpS crystal will be useful as a composition in medical, clinical, research, industrial and pharmaceutical applications as described herein.

In another particular embodiment, a crystal composition of the present invention is modified by creating an immobilized AcpS crystal of the invention, such as, for example, by linking AcpS crystals to each other and/or to a solid substrate (using any number of linking reagents and linking methods or linking means known in the art. For example, such as those described in U.S. Pat. No. 6,004,768; U.S. Pat. No. 6,042,824; WO 01/1638A2; Weygand, et al 2000 J. Mater Chem. 10:141-148; Pum, et al 2000 Nanotechnology 11: 100-107; and Ullman, 1991 An Introduction to Ultrathin Organic Films: From Langmuir-Blodgett to Self Assembly, Academic Press). Immobilized enzyme crystals retain their catalytic activity and have improved characteristics such as greater stability and resistance to degradation (see, e.g., S. J.. Bayne et al., “Enzymatically Active, Cross-Linked Pig Heart Lactate Dehydrogenase Crystals”, Carlsberg Res. Comm., 41, pp. 211-216 (1976); A. Dyer et al., “A Thermal Investigation of the Stability of Crystalline Cross-Linked Carboxypeptidase A”, Thermochimica Acta, 8, pp. 455-464 (1974); J. V Hupkes, “Practical Process Conditions for the Use of Immobilized Glucose Isomerase,” Starch, 30, pp. 24-28 (1978); P.J. Kasvinsky et al., “Activity of Glycogen Phosphorylase in the Crystalline State”, J. Biol. Chem., 251, pp. 6852-6859 (1976); and H. Mrsten et al., “Catalytic Activity of Non-Cross-Linked Microcrystals of Aspartate Aminotransferase in Poly (ethylene glycol)”, Biochem. J., 211, pp. 427-434 (1983)). Such immobilized AcpS crystals are also useful as a composition in medical, clinical, research, industrial and pharmaceutical applications as described herein.

In a more particular embodiment, an immobilized crystal composition of the invention is produced by linking an AcpS crystal with a bifunctional linking reagent, such as, for example, glutaraldehyde. This results in the stabilization of the crystal lattice contacts between the individual enzyme catalyst molecules constituting the crystal. As a result, the crystals are immobilized and function at elevated temperatures, extremes of pH and in harsh aqueous, organic, or near-anhydrous media, including combinations of such conditions. Therefore, in one embodiment, an immobilized crystal composition of the invention functions in environments incompatible with the functional integrity of corresponding uncrystallized, unimmobilized, and/or its corresponding enzyme in the native state.

In still further embodiments, immobilization of an AcpS crystal further encompasses without limitation, an AcpS crystal of the invention being: caged (such as, e.g., Schnur, J. M. 1993. Lipid tubules: A paradigm for molecularly engineered structures. Science 262: 1669-1676; Schnur, et al. 1994. Biologically engineered microstructures—controlled-release applications. J. Controlled Release 28 (1-3)(Jan.): 3-13); encapsulated (such as, e.g., in foams, fluids, gels, polymers or membranes see, e.g., WO 00/18972AD; WO 98/23734); tethered (such as, e.g., by an antibody or antibody binding fragment); coupled; bound to bacterial S-layers in predictable and geometrically well defined ways (see, e.g., Pum & Sleytr 1999 Trends in Biotechnology 17:8-11 and the references cited therein); coated (such as, e.g., with nanoparticles or polyelectrolyte molecules); electrostatically bound; layered; (such as, e.g., in 2D crystal arrangements); formed by placing AcpS proteins beneath lipid monolayers then subsequently stabilized into a durable geometric arrangement; covalently, or non-covalently bonded using any method in the art (such as, e.g., WO 00/77281 Al; U.S. Pat. No. 5,091,187; U.S. Pat. No. 5,716,709; McPherson, A 1998. Crystallization of Biological Macromolecules, Cold Spring Harbor Laboratory Press, Cold Spring Harbor, N.Y. or; Gilliland, G. L. 1988. A biological macromolecule crystallization database: a basis for a crystallization strategy. J. Cryst. Growth 90: 51-59; Sára, et al. 1996 in Crystalline Bacterial Cell Surface Proteins (Sleytr, et al. Eds), pp. 133-159, R. G. Landesall).

Immobilized AcpS crystals can be lyophilized, producing AcpS compositions which can be stored at non-refrigerated (room) temperatures for extended periods of time, and which can be reconstituted in aqueous, organic, or mixed aqueous organic solvents of choice, without the formation of amorphous suspensions and with minimal risk of denaturation. The lyophilization of such resulting compositions provides a means of improving storage, handling, and manipulation properties of AcpS crystals of the invention. Use of the term “AcpS crystal” herein encompasses both immobilized and unimmobilized forms as described herein unless the context would clearly dictate otherwise to one skilled in the art to which it applies.

Using such crystal compositions, the present invention also encompasses methods of making selected products with them. For example, in the research and pharmaceutical area, AcpS crystals can be used in the preparation of acyl-carrier proteins (ACP) or ACP analogs or ACP derivatives such as, for example, ACP analogs with modified phosphopantetheines (see, e.g., Gehring, et al., 1997 “Ability of Streptomyces Acyl Carrier Proteins and Coenzyme A Analogs to Serve as Substrates in vitro for E. coli holo-ACP Synthase” in Chemistry and Biology 4:17). ACP analogs can be used to probe mechanistic questions in enzymes involved in the biosynthesis of fatty acids and they can also be used for commercial or pharmaceutical applications, such as, e.g., to facilitate the production of polyketides and polyketide derivatives, which have been shown to have many useful functions (for example, e.g., the polyketide antibiotic erythromycin A is used against Gram-positive bacterial infections, particularly against penicillin-resistant infections. Another polyketide, Amphotericin-B, is used primarily as an antifungal agent. The tetracyclines are broad-spectrum antibiotic polyketides that have activity against both Gram-positive and Gram-negative bacteria. Another important polyketide is doxorubicin (also known as Adriamycin), which is a widely used antitumor agent, particularly against solid tumors. Further, another interesting composition is the polyketide derivative lovastatin, a compound that has found wide use as a cholesterol reducing agent under the trade name of Mevacor®). Due to the broad substrate specificity of the AcpS enzyme, AcpS crystals themselves can also be used in the production of pharmaceutical reagents such as the above mentioned polyketides (see, e.g., Suo, et al. 2001 Proc Natl Acad Sci U S A 98(1):99-104). Accordingly, AcpS crystalline compositions are useful in the manufacture or catalysis of selected products such as for research, pharmaceutical, or industrial applications.

AcpS Crystalline Compositions for Use in Microelectronics

In still another embodiment, an AcpS crystal of the invention can be used, for example, as a fabrication material in the process, manufacture, and/or production of a microelectronic device. In one embodiment, AcpS protein crystals are employed using saturated solutions in the formation of a two-dimensional (2D) crystalline array on a solid support (such as, e.g., a silicon wafer). In 2D-nucleation growth, a 2D island is first nucleated on a flat crystal face. The 2d crystal island is a collection of molecules that are usually a single growth layer in height. Such an application of a crystal of the invention is useful as, for example, a nanometre-thick resist in semiconductor technologies and as a template for the formation of regularly arranged nanoparticles for applications in molecular electronics (see, e.g., Share, et al. Gradient composite replicas from protein crystal layer templates produced by pulsed laser deposition. PTB-Berichete, F-39, pp. 8-16; “Biologically Derived Nanometer-Scale Patterning on Chemically Modified Silicon Surfaces,” B. W. Holland, K. Douglas, N. A. Clark, Mat. Res. Soc. Symp. Proc. 330, 121 (1994); “Transfer of Biologically-Derived Nanometer-Scale Patterns to Smooth Substrates,” K. Douglas, G. Devaud, N. A. Clark, Science 257, 642 (1992); Abstract Y13.14 Laser Seeding for Biomolecular Crystallization Bancel, et al., from the 1998 March Meeting of The American Physical Society Los Angeles, Calif. showing a novel seeding technique in which a laser beam is used to select and transfer microscopic seed crystals in a growth solution; Allara, D. L. 1996. Nanoscale structures engineered by molecular self-assembly of functionalized monolayers. In Nanofabrication and Biosystems. Ed. H. C. Hoch, L. W. Jelinski, and H. G. Craighead. New York: Cambridge University Press, U.S. Pat. No. 5,597,457; WO 01/16328 for the application of proteins to a substrate. Additional references can be found in the work of Whitesides and colleagues which is described and referenced in WO 97/06468 and in the following patents: WO9954786 (A1) Elastomeric mask and use in fabrication of devices, including pixelated electroluminescent displays; WO9629629 (A2, A3), Microcontact printing on surfaces and derivative articles; W09707429 (A1), Self-assembled monolayer directed patterning of surfaces; WO9858967 (A1), Self-assembling peptide surfaces for cell patterning and interactions; U.S. Pat. No. 6,197,515, Molecular recognition at surfaces derivatized with self-assembled monolayers; U.S. Pat. No. 6,180,239, Microcontact printing on surfaces and derivative articles; U.S. Pat. No. 5,976,826, Device containing cytophilic islands that adhere cells separated by cytophobic regions; U.S. Pat. No. 5,900,160, Methods of etching articles via microcontact printing; U.S. Pat. No. 5,512,131, Formation of microstamped patterns on surfaces and derivative articles; U.S. Pat. No. 5,620,850, Molecular recognition at surfaces derivatized with self-assembled monolayers; U.S. Pat. No. 5,776,748, Method of formation of microstamped patterns on plates for adhesion of cells and other biological materials, devices and uses therefor.).

One advantage of using an AcpS crystal in microfabrication and in microlithography is that it permits the design and realization of smaller transistors that can be fabricated on a computer chip. Reduction in transistor size permits greater numbers of transistors in a defined area and thus faster data processing times that use less energy. Such reductions in transistor size support “Moore's Law,” which describes a predictable and continuing trend in the development of memory chip performance so that each new computer memory chip contains roughly twice as much capacity as its predecessor. Use of AcpS crystals in microchip fabrication provides another method for realizing “Moore's Law.”

Methods of fabricating microchips using proteins such as the AcpS crystals of the invention can be accomplished without undue experimentation using any known method in the art of protein engineering and microlithography. For example, one technique used in micro- and nanoelectronic applications is a microlithographic procedure using deep ultraviolet (DUV) laser irradiation for transferring (sub) micrometer patterns on a substrate to pattern 2D protein layers on silicon wafers (see, e.g., Pum, et al. 1996 Colloids Surf. B: Biointerfaces 8, 157-162; Pum, D. et al. 1997 Microelectron. Eng. 35, 297-300; and Calvert, J. M. 1993 J. Vaccine Sci. Technol. B 11, 2155-2163 and Pum & Sleytr 1999 Trends in Biotechnology 17:8-11). Typically, patterns are formed on 2D layers by bringing a chromium mask (on quartz glass) into direct contact with the 2D protein crystal layer on a silicon wafer. Upon irradiation with ArF pulses (DUV emitted after excitation of argon-fluoride gas in an excimer laser; wavelength 193 nm, dose ˜100 mJ per squared cm, pulse duration ˜8 nsec), the protein layer is completely removed in the exposed areas (i.e. without the mask) but retains its structural and functional integrity in the unexposed regions (i.e. masked areas). The masked or unexposed regions can subsequently be used to selectively bind other biologically functional molecules (here, for example, such as AcpS to form an AcpS/apo-ACP binding complex) or to be reinforced for subsequent reactive ion etching using any techniques in the art. Different etching rates between exposed and unexposed regions are necessary for reactive-ion etching and can obtained by reinforcing the protein layer (here, the AcpS layer) with silicon, a procedure known as silylation (see, e.g., Shaw, et al. 1989 J. Vaccine Sci. Technol. B 7, 1709). Using similar techniques, crystal protein layers are currently being produced for use as novel high-performance resists with thickness in the 10 nm range (see, e.g., Pum & Sleytr 1998 in Biological Molecules In Nanotechnology: The Convergence of Biolechnology, Polymer Chemistry and Materials Science, IBC Library Series pp.139-143 Southborough: International Business Communication, Inc.; Pum, et al. 1996 Colloids Surf. B: Biointerfaces 8, 157-162; WO 01/16328 Pum, et al. 1997 Microelectron. Eng. 35, 297-300).

Using these and similar techniques, one of skill in the art could use an AcpS crystal of the invention to serve as a fabrication mask and/or a template for improvements in silicon nano- and micro-fabrication technology. This is especially true in light of the recent successes in immobilizing biological nanoparticles (e.g., enzymes, antibodies, etc.) on these surfaces, leading to functional devices that can be employed as sensors, nanoelectrodes, prosthetic devices, or nanomachines.

AcpS Crystalline Compositions for Use in Biosensors

In another embodiment, a crystal composition of the invention is employed as a biomaterial for use in a biosensor. Biosensors use biomaterials, such as, e.g., an AcpS crystal of the application, to detect various substances of clinical, industrial, and other interest (see, e.g., Hall, E., Biosensors, Open University Press (1990)).

Biomaterials, such as those comprising a crystal of the invention (fabricated as described herein), can be integrated with a solid support using any number of strategies such as, e.g., those previously described herein and the following:

(1) by incorporation in organic or inorganic polymers that are associated with the solid matrices (see, e.g., Cosnier, S. (1997) Electropolymerization of amphiphilic monomers for designing amperometric biosensors. Electroanal. 9, 894-902; Kranz, C. et al. (1998) Controlled electrochemical preparation of amperometric biosensors based on conducting polymer multilayers. Electroanal. 10, 546-552; Willner, I. et al. (1992) Bioelectrocatalyzed reduction of nitrate utilizing polythiophene bipyridinium enzyme-electrodes. Bioelectrochem. Bioenerg. 29, 2945; Heller, A. (1992) Electrical connection of enzyme redox centers to electrodes. J. Phys. Chem. 96, 3579-3587; Gregg, B. A. and Heller, A. (1990) Cross-linked redox gels containing glucose-oxidase for amperometric biosensor applications. Anal. Chem. 62, 258-263; Gregg, B. A. and Heller, A. (1991) Redox polymer-films containing enzymes. 1. A redox-conducting epoxy cement—synthesis, characterization and electrocatalytic oxidation of hydroquinone. J. Phys. Chem. 95, 5970-5975; Gregg, B. A. and Heller, A. (1991) Redox polymer-films containing enzymes. 2. Glucose-oxidase containing enzyme electrodes. J. Phys. Chem. 95, 5976-5980; Walcarius, A. (1998) Analytical applications of silica-modified electrodes—a comprehensive review. Electroanal. 10, 1217-1235; Tsionsky, M. et al. (1994) Sol-gel-derived ceramic carbon composite electrodes—introduction and scope of applications. Anal. Chem. 66, 1747-1753);

(2) by the generation of physical blend composites between a biomaterial and an electronic element material (see, e.g., Wang, J. and Naser, N. (1994) Improved performance of carbon-paste amperometric biosensors through the incorporation of fumed silica. Electroanal. 6, 571-575; Hale, P. D. et al. (1991) Amperometric glucose biosensors based on redox polymer-mediated electron-transfer. Anal. Chem. 63, 677-682; Hale, P. D. et al. (1989) A new class of amperometric biosensor incorporating a polymeric electron-transfer mediator. J. Am. Chem. Soc. 111, 3482-3484; Kaku, T. et al. (1994) Amperometric glucose sensors based on immobilized glucose oxidase polyquinone system. Anal. Chem. 66, 1231-1235) e.g. redox enzymes in carbon paste blends); or

(3) by the incorporation of biomaterials in membrane assemblies organized on the transducers (Kinnear, K. T. and Monbouquette, H. G. (1993) Direct electron-transfer to Escherichia coli fumarate reductase in self-assembled alkanethiol monolayers on gold electrodes. Langmuir 2255-2257).

The functionalization of solid supports with monolayers, multilayers of controlled thickness, or thin film assemblies of biomaterials, reveals several attractive features for bioelectronic devices such as biosensors. Besides the fundamental feasibilities to structurally control, manipulate and address biomaterials such as protein crystals in 2D or thin 3D crystal arrays, these configurations exhibit practical advantages because they lack diffusional barriers, and biological processes that occur on the surface are rapidly translated to electronic outputs of a biosensor. The chemistry of surface modification of solid interfaces for use with monolayer and multilayer arrays has been addressed in several comprehensive review articles (see, e.g., Langmuir 2255-2257; Albery, W. J. and Hillman, A. R. (1982). Modified electrodes. Annu. Rep. Prog. Chem. Sect. C 78, 377-437; Murray, R. W. (1984) Chemically modified electrodes. In Electroanalytical Chemistry (Vol. 13) (Bard, A. J., ed.), pp. 191-368, Marcel Dekker, New York; Wrighton, M. S. (1986) Surface functionalization of electrodes with molecular reagents. Science 231, 32-37; Finklea, H. O. (1966) Electrochemistry of organized monolayers of thiols and related molecules on electrodes. In Electroanalytical Chemistry (Vol. 19) (Bard, A. J. and Rubinstein, I., eds), pp. 109-335, Marcel Dekker, New York; Zhong, C. J. and Porter, M. D. (1995) Designing interfaces at the molecular level. Anal. Chem. 67, 709A-715A.

For example, functionalized thiolate monolayers associated with gold electrodes have been used as base interfaces for the covalent linkage of biomaterials, for example, enzymes (Shoham, B. et al. (1995) A bilirubin biosensor based on a multilayer network enzyme electrode. Biosens. Bioelectron. 10, 341-352; Riklin, A. and Willner, I. (1995) Glucose and acetylcholine sensing multilayer enzyme electrodes of controlled enzyme layer thickness. Anal. Chem. 67, 4118-4126) or antibodies (Cohen, Y. et al. (1996) Modified monolayer electrodes for electrochemical and piezoelectric analysis of substrate-receptor interactions; novel immunosensor electrodes. J. Electroanal. Chem. 417, 65-75). Alternatively, functionalized siloxane films immobilized onto oxide solid supports can be used to assemble biomaterials on the oxide surfaces (Zou, C. F. and Wrighton, M. S. (1990) Synthesis of octamethylferrocene derivatives via reaction of (octamethylferrocenyl)methyl carbocation with nucleophiles and application to functionalization of surfaces. J. Am. Chem. Soc. 112, 7578-7584; Murray, R. W. (1980) Chemical modified electrodes. Acc. Chem. Res. 13, 135-141; Abruña, H. D. (1998) Coordination chemistry in 2 dimensions - chemically modified electrodes. Coord. Chem. Rev. 86, 135-189).

Using such teachings and knowledge in the art, one of ordinary skill could use a crystal composition of the invention as a biomaterial of a biosensor, for use in detecting and/or quantitating an analyte of interest, such as an analyte in a fluid, such as, for example a biological sample (e.g., a bodily fluid, such as, e.g., blood, urine, lavage, sputum, etc.), chemical and laboratory reaction media, organic media, water, culture media, foodstuffs, and beverages. In some instances, the fluid in question can be a gas.

In one particular embodiment, an AcpS crystal is used as a biomaterial in a biosensor and brought into contact with a fluid to detect an analyte. The analyte can be measured directly (such as, e.g., blood glucose level) or indirectly (e.g., such as by detecting or quantitating a substance which is a reactant (product or substrate) in a reaction in which the analyte of interest participates. In either case, the crystal composition is able to interact with the analyte or a substance that is a reactant in a reaction (such as, e.g., an analyte-binding partner that forms a binding complex with an AcpS crystal composition) in which the analyte also participates. The interaction of a crystal form of the enzyme of the invention with an analyte results in a detectable change such as, e.g., any of the following non-limiting examples: a change in pH; a colormetric change; a production of reaction product; a weight change; a change in an interference pattern (such as an interference pattern produced by a reflected wave, such as, e.g., a wave of light, a photon, a sound wave, a pressure wave, etc.); the production of light; a piezoelectric effect; a change in conductivity (such as, e.g., conductivity of: heat, light, ionic, electric, gravimetric, etc.); a change in heat; a change in electrical potential. Any such change detected and/or quantitated by any appropriate detecting means such as, e.g., without limitation: a pH electrode, a light or a heat sensing device, a means for measuring an interference pattern (e.g., such as a means for analyzing an interference pattern produced by coherent light), a means for measuring a fluctuation in weight; a means for measuring an enzymatic assay; a gravimetric detecting means; a means for measuring electrical charge; and/or a means for detecting a formation of an enzyme/binding partner complex is also encompassed herein. Any means useful for detecting a change resulting from an enzyme interaction with an analyte can be used and is encompassed by the present application. Typically a biosensor of the present invention comprises a crystal AcpS composition and a retaining means for the crystal which allows contact between the crystal(s) and an analyte of interest or a substance in a sample (e.g., such as a biological, organic, or inorganic sample) to produce a detectable change, such as, e.g., a change which is a reaction in which the analyte of interest participates.

In a more particular embodiment, a biosensor of the invention comprises amperometric detection of binding of an analyte, such as a binding agent, to a crystal composition of the invention (such as, e.g., a biomaterial comprising a 2D crystalline protein formation or a thin 3D crystal array). In this embodiment, the biosensor surface is an electrode, and the biomaterial is sufficiently closely packed and ordered (such as, e.g., in thin layer) to form an effective barrier to a current (across the biomaterial) mediated by, for example, a redox ion species in an aqueous solution in contact with the biomaterial layer. Binding of a binding agent to the biomaterial, e.g., the 2D crystalline protein formation or a or thin 3D crystal array, is sufficient to affect current flow (such as, e.g., current flow mediated by a redox species) so that binding to a crystal causes a shape change (such as, e.g., through cracking of the crystal and/or by shifting the order of the crystal packing), which subsequently permits a change in current flow across or through the biomaterial. In one such embodiment, a chamber in an apparatus is adapted to contain an aqueous solution of redox species in contact with a layer of a crystal of the invention, and the detector includes a circuit for measuring ion-mediated current across the layer in response to a binding event occurring between an AcpS crystal composition and an anaylate to form a binding complex with said crystal. The triggering event in such a biosensor is the binding of an anaylate to the AcpS crystal. Without being bound by theory, this binding can perturb the ordered structure of the biomaterial layer (or a sufficient number of individual crystals) to permit movement of redox species through the biomaterial to produce a detectable current or current change. In one example, the biosensor detects a binding event as an increase or decrease in current across an electrode, i.e., between working and counter electrodes. By analogy to a transistor, the redox solution serves as a “source,” the biomaterial layer as a “gate,” and the underlying electrode as the “drain.” Current in the biosensor “transistor” is initiated by applying a threshold voltage to the gate. In this embodiment, current is initiated by a stimulus to the monolayer “gate,” i.e., by binding to a crystal.

One extant example of an amperometric biosensor uses glucose oxidase modified with a ferrocene conductor. The modified enzyme is self-assembled onto a porous gold-black electrode to detect glucose in solution by exploiting an electron transfer pathway of the enzyme as a sensor (Aizawa, et al., 1996. Molecular assembly technology for biosensors. In Nanofabrication and Biosystems: Integrating Materials Science, Engineering And Biology. Ed. Hoch, et al., New York: Cambridge University Press).

In another embodiment, a biosensor is designed for gravimetric detection of binding of a binding agent to a crystal of the invention. In such an embodiment, the biosensor surface is a piezoelectric AcpS crystal.

The detector functions by generating a surface acoustic wave in the crystal and then detecting a shift in wave frequency, velocity, or resonance frequency of a surface acoustic wave produced in response to a binding event occurring between the crystal and an anaylate forming a binding complex with said crystal. Surface acoustic waves are generated in the crystal or crystal layer by any oscillator means. Not being bound by theory, but, according to currently accepted piezoelectric principles, the change in mass of the biomaterial resulting from binding of an AcpS crystal alters the frequency, resonance frequency, and/or wavelength of the surface acoustic waves, and at least one of these wave characteristics is measured by a detector means. The oscillator and detector collectively form a detector means for detecting binding of a binding agent to an AcpS crystal on the biosensor surface. Details of associated detector means in gravimetric biosensors are given, for example, in U.S. Pat. Nos. 5,478,756 and 4,789,804, and in PCT application WO 96/02830.

In still another embodiment, a biosensor encompassed herein is designed for optical surface plasmon resonance (SPR) detection of binding of a binding agent to a crystal composition of the invention. In this embodiment, the biosensor surface is a transparent dielectric substrate coated with a thin metal layer on which a 2D crystal layer or thin 3D crystal composition layer of the invention is formed so that the substrate and metal layer form a plasmon resonance interface. The detector functions to excite surface plasmons at a plasmon resonance angle which is dependent on the optical properties of the metal film and attached biomaterial layer, and to detect a shift in plasmon resonance angle in response to a binding event occurring between a crystal composition and an anaylate to form a binding complex.

Typical elements of a surface plasmon resonance (SPR) biosensor comprise: an open-top chamber in the biosensor which contains a waveguide composed of a dielectric film and a thin evaporated metal film constructed to support surface plasmon waves at the dielectric/metal film interface. The waveguide surface forms a biosensor surface having a crystal layer (such as, e.g., a crystalline 2D layer of AcpS or other finally deposited crystalline AcpS biomaterial). A light source directing a divergent light beam onto the biosensor surface through a lens. At some region along the length of the biosensor surface, the beam angle strikes the surface at an absorption angle at which absorption from the evanescent wave by surface plasmons occurs. The absorption angle will shift with changes in the composition of the biomaterial near the interface, that is, in response to binding events occurring at the crystal surface. The intensity of reflected light from each region along the biosensor surface is monitored by a photosensor whose photosensing grid is matched to specific detector surface regions, and which is operatively connected to an analyzer means. The light source and photosensor in this embodiment are referred to herein as biosensor means. In operation, the SPR absorption angle on the surface of the biomaterial is measured before and after addition of an analyte, with the measured shift in angle being proportional to the extent of binding of an analyte to form an AcpS/binding partner complex.

In an additional embodiment, a biosensor encompassed by the invention employs optical detection of binding of a binding agent to a crystal of the invention. The optical detector functions by irradiating a biomaterial surface of the biosensor with a light beam (such as, e.g., coherent light) and then detecting a change in the optical properties of the biomaterial. In one example, the detecting means uses ellipsometry (an optical physical measurement technique which can be used to measure small changes of refraction index at surfaces with high sensitivity, by measuring changes in elliptisity of polarized light, such as those caused by the presence of analyte biomolecules on the surface of a biomaterial of the invention) to detect binding.

Such biosensor applications can be used herein with a crystal composition of the present invention. More specifically, crystallized AcpS can be used in a biosensor application as described to detect the presence of a pathogen, such as e.g., but without limitation, the a bacterial or fungal agent that can cause a disease, disorder, condition, syndrome, or symptom such as, e.g., the following agents: Gram-Negative and Gram-positive bacteria and bacterial families and fungi such as: Actinomycetales (e.g., Corynebacterium, Mycobacterium, Norcardia), Cryptococcus neoformans, Aspergillosis, Bacillaceae (e.g., Anthrax, Clostridium), Bacteroidaceae, Blastomycosis, Bordetella, Borrelia (e.g., Borrelia burgdorferi), Brucellosis, Candidiasis, Campylobacter,Coccidioidomycosis, Cryptococcosis, Dermiatocycoses, E. coli (e.g., Enterotoxigenic E. coli and Enterohemorrhagic E. coli), Enterobacteriaceae (Klebsiella, Salmonella (e.g., Salmonella typhi, and Salmonella paratyphi), Serratia, Yersinia), Erysipelothrix, Helicobacter, Legionellosis, Leptospirosis, Listeria, Mycoplasmatales, Mycobacterium leprae, Vibrio cholerae, Neisseriaceae (e.g., Acinetobacter, Gonorrhea, Menigococcal), Meisseria meningitidis, Pasteurellacea Infections (e.g., Actinobacillus, Heamophilus (e.g., Heamophilus influenza type B), Pasteurella), Pseudomonas, Rickettsiaceae, Chlamydiaceae, Syphilis, Shigella spp., Staphylococcal, Meningiococcal, Pneumococcal and Streptococcal (e.g., S. typhimurium, Streptococcus pneumoniae and Group B Streptococcus).

For example, it has been shown that during infections AcpS proteins are specifically upregulated (e.g., the fadB gene of S. typhimurium (which encodes AcpS I) was identified in a screen for genes expressed during infection using in vivo expression technology (Mahan, et al. 1995 Proc Natl Acad Sci USA 92:669-673; and Mahan, et al. 1993 Infect Agents Dis 2:263-268) and the fadF gene also of S. typhimurium (which encodes AcpS II) was induced after the bacteria was phagocytosised by macrophages (DiRusso, et al. 1999 Progress in Lipid Research 38:129-197)). Accordingly, biological samples comprising an infectious agent and/or constituents thereof could be applied to a biosensor on a microchip for analytical, quantitative, and/or qualitative chemical analysis. Such “lab-on-a-chip” functions can be used for example, to determine the presence or level (compared to a control) of CoA and/or apo-ACP in a sample thus indicating the presence of an infectious agent. In another embodiment, AcpS crystals are used in a biosensor to determine binding agents that can modify an AcpS activity, for example, such as by acting as an agonist or antagonist.

Further embodiments of the present invention include computer-readable media encoded with data representing the atomic coordinates of the three-dimensional structure of Streptococcus pneumoniae AcpS, or computer-readable media having stored thereon a model comprising the three-dimensional structure of the catalytic active site domain of Streptococcus pneumoniae AcpS. As used herein, the term “computer-readable medium” refers to any medium that can be read and accessed directly by a computer. Such media include, but are not limited to, magnetic storage media, such as floppy discs, hard disc storage media, and magnetic tape; optical storage media, such as optical discs or CD-ROMs; electrical storage media, such as RAM and ROM; and hybrids of these categories, such as magnetic/optical storage media. Those of ordinary skill in the art can readily appreciate how any of the presently known computer-readable media can be used to create a manufacture comprising a computer-readable medium having recorded thereon an amino acid or nucleotide sequence and/or atomic coordinates of the present invention.

Other features of the present invention will become apparent from the following examples, which are for illustrative purposes only, and which are not intended to limit the invention in any way.

### EXAMPLE 1

### Cloning of the S. pneumoniae acpS and acpP Genes; Expression and Purification of ACPS and ACPP

To understand better the function of AcpS in Streptococcus pneumoniae, a sphere-shaped, Gram-positive bacterium and major human pathogen of the upper respiratory tract, and to explore AcpS as an antibacterial target, the acpS and acpP genes of S. pneumoniae are cloned and expressed, and the gene products characterized. As disclosed below, the results disclosed herein demonstrate that S. pneumoniae AcpS shares many biochemical properties with E. coli AcpS. In addition, the results suggest that AcpS proceeds by an ordered reaction mechanism with the initial formation of the enzyme-apo-ACP intermediate from apo-ACP, followed by the transfer of 4′-phosphopantetheine from CoA to the apo-ACP of the complex. Finally, both acpS and acpP form complex operons with the genes whose functions are not required for fatty acid biosynthesis.

AcpS, an enzyme essential for bacterial fatty acid synthesis, catalyzes the transfer of 4′-phosphopantetheine from CoA to apo-ACP to form holo-ACP along with the production of 3′,5′-ADP, and is an attractive target for the development of antibacterial drugs. The structure of AcpS reveals an α/β fold, and demonstrates that the trimeric structure of the enzyme appears to be essential for the AcpS activity. These results represent the first structural determination of the interaction between the AcpS enzyme and its product, 3′,5′-ADP. These data provide a starting point for structure-based drug design efforts that should identify novel AcpS inhibitors with potent antibacterial activity.

To solve the structure of the S. pneumoniae AcpS, the protein is purified to homogeneity from an E. coli expression host using a three-step purification method (McAllister et al., 2000). The purified AcpS is crystallized as described below. Crystals of AcpS are obtained after 4-5 days at room temperature. Data are collected from these crystals, and the structure of AcpS is then solved by the multiple anomalous dispersion method (Hendrickson et al., 1991) (MAD) using selenomethionine-substituted protein and exploiting non-crystallographic 3-fold averaging.

Materials

Unless specified otherwise, all fine chemicals are from Sigma Chemical Company (St. Louis, Mo.). All fast protein liquid chromatography (FPLC) resins and columns used for protein purification, and strains and reagents for construction, expression, and purification of GST-fused proteins, are obtained from Amersham Pharmacia Biotech (Piscataway, N.J.). Luria Bertani (LB) broth medium is purchased from Bio101, Inc. (Vista, Calif.). All polyacrylamide gels and reagents are purchased from Novex (San Diego, Calif.). SYPRO Orange and Bradford protein assay reagents are purchased from BIO-RAD (Hercules, Calif.), and Sulfo-EGS (ethylene glycolbis(succinimidylsuccinate)) is obtained from Pierce (Rockford, Ill.). ³H-CoA (specific activity, 1.5 Ci/mmol) is custom-synthesized by NEN Life Science Products (Boston, Mass.).

### Cloning and Expression of the acpS and acpP Genes of S. pneumoniae (hex-) R6

The acpS and acpP genes are cloned from S. pneumoniae by PCR using the same reagents, plasmids, and cell lines used for cloning and expression as described in Zhao et al., 1999. The sequences of the S. pneumoniae acpS gene and AcpS protein are disclosed in U.S. Pat. No. 6,060,282, issued May 9, 2000 (note also GenBank accession number AF276617). The acpS gene, 369 bp long, encodes a protein consisting of 122 amino acid residues, with a predicted molecular weight of 13.7 kDa (accession number AF276617). As disclosed in this patent, the deduced amino acid sequence of the S. pneumoniae AcpS protein consists of 122 amino acids, as follows (SEQ ID NO:1):

Met Arg Met Ile Val Gly His Gly Ile Asp Ile Glu Glu Leu Ala Ser
Ile Glu Ser Ala Val Thr Arg His Glu Gly Phe Ala Lys Arg Val Leu
Thr Ala Gln Glu Met Glu Arg Phe Thr Ser Leu Lys Gly Arg Arg Gln
Ile Glu Tyr Leu Ala Gly Arg Trp Ser Ala Lys Glu Ala Phe Ser Lys
Ala Met Gly Thr Gly Ile Ser Lys Leu Gly Phe Gln Asp Leu Glu Val
Leu Asn Asn Glu Arg Gly Ala Pro Tyr Phe Ser Gln Ala Pro Phe Ser
Gly Lys Ile Trp Leu Ser Ile Ser His Thr Asp Gln Phe Val Thr Ala
Ser Val Ile Leu Glu Glu Asn His Glu Ser

It is not known if the AcpS protein as naturally produced by S. pneumoniae begins with Met Arg or Met Ile. For the purpose of the studies herein, a primer corresponding to the second Met start codon at the N-terminus of the foregoing sequence is designed and used for cloning the S. pneumoniae acpS gene. Thus, the acpS coding sequence used for expression of the protein herein produces an AcpS protein lacking the first two amino acid residues (Met Arg) of the foregoing sequence. Specifically, the following PCR primers are designed and used to amplify the acpS gene for cloning into E. coli expression systems. The 5′ PCR primer (5′-CGCGGATCCCATATGATAGTTGGACACGGAATTG -3′; SEQ ID NO:2) is designed at the second ATG start codon of acpS, and contains BamHI and NdeI sites for cloning purposes. The 3′ PCR primer (5′-CGCGGATCCCTAGCTTTCATGAATTTCCTCC -3′; SEQ ID NO:3) is designed at the stop codon of acpS, and contains a BamHI site after the stop codon. Using these primers, acpS is PCR amplified from S. pneumoniae for 25 cycles under the conditions described in Zhao et al., 1999. Five PCR reaction products are combined, and a portion of the pooled PCR products is digested with BamHI. The BamHI-digested PCR fragment is cloned into pCZA342, a low copy number plasmid (Baltz et al., 1997) that is digested with BamHI and dephosphorylated with calf intestinal alkaline phosphatase. acpS from several pCZA342 clones is sequenced, and a clone containing the consensus acpS gene sequence is used for constructing expression systems. This pCZA342 clone is digested with NdeI and BamHI. The NdeI-BamHI DNA fragment containing acpS is subcloned into pET-11a (Novagen). The resulting construct is designated as pRBP-19. The pCZA342 clone is also digested with BamHI, and the BamHI fragment of acpS is subcloned into pGEX-2T, resulting in pRBP-20.

To clone the acpP gene, the following PCR primers are used for amplification. 5′ PCR primer:

5′-CGCGGATCCCATATGACAGAAAAAGAAATTTTTGACCGTATTG -3′ (SEQ ID NO:4); 3′ PCR primer:

5′-CGCGGATCCGAATTCCTATTTTCCTTGAATGATTTTAACCACATC-3′ (SEQ ID NO:5). Using these primers, acpP is PCR amplified from S. pneumoniae as described above. The PCR products are digested with BamHI. The BamHI-digested PCR fragment is cloned into pCZA342. The pCZA342 clone is digested with NdeI and BamHI. The NdeI-BamHI DNA fragment containing acpP is subcloned into pET-11a (Novagen), resulting in pRBP-16.

Purification of AcPS and ACP of S. pneumoniae

LY128 (E. coli BL21 (pLysS)/pRBP-19) is first grown at 35° C. overnight in LB broth medium supplemented with 100 μg/ml ampicillin. The overnight culture (40 ml) is then inoculated into 1000 ml of LB medium supplemented with ampicillin, and grown at 33° C. with shaking at 250 rpm until an OD590 of 0.5-0.6 is reached. The culture is induced with 1 mM isopropyl-1-thio-β-D-galactopyranoside (IPTG) for 3 hr. Cells are harvested by centrifugation at 4500×g at 4° C. for 8 min, washed twice in phosphate buffered saline (PBS), resuspended in 50 mM citrate phosphate pH 6.0, and disrupted by passing twice through a French pressure cell. The resulting cell extract is centrifuged at 160,000×g for 40 min at 4° C. The supernatant fraction is collected and applied to a 15S Source S cation exchange column (2.5×8 cm) equilibrated with 50 mM citrate phosphate, pH 6.0 (buffer A). The column is washed with buffer A, and eluted with a linear gradient of 0-1.0M KCl in buffer A. Fractions (7 ml each) are collected. The presence of AcpS in the fractions is detected by SDS-PAGE analysis (16% tricine gels) (Laemmli, 1970). The fractions containing AcpS are pooled, and applied to an S-100 Sepharose preparative gel filtration fast protein liquid chromatography column (5.0×60 cm) equilibrated with 50 mM Tris-HCl, pH 7.0, 100 mM KCl. The fractions containing AcpS are collected, adjusted with glycerol to a final concentration of 15% (v/v), and stored in small aliquots at −70° C. Protein concentration is determined using a protein assay kit (Bio-Rad), with BSA as a standard (Bradford, 1976).

LY135 (E. coli XL1 Blue (mRF′)/pRBP-20) is grown, induced, harvested, disrupted, and centrifuged as above. The supernatant fraction is applied to a glutathione Sepharose 4B column (10 ml) equilibrated with 100 ml of PBS. The column is washed with 100 ml of PBS, and the GST-AcpS fusion protein is eluted with 10 mM glutathione in PBS. Fractions are analyzed by SDS-PAGE (12% glycine) and those fractions containing GST-AcpS are pooled, dialyzed against 50 mM Tris-HCl, pH 7.0 (4 liters), adjusted with glycerol to the final concentration of 15% (v/v), and stored at −70° C. as described above.

LY140 (E. coli BL21 (pLysS)/pRBP-16) is grown, induced, harvested, and disrupted as described above. The resulting cell extract is also centrifuged as described above. The supernatant fraction is collected and applied to a 15S Source Q-column (2.5×8 cm) equilibrated with 50 mM Tris-HCl, pH 8.0, 100 mM KCI (buffer C). The column is washed with 100 ml of buffer C, and eluted with a linear gradient of 0.0-1.0M KCl in buffer C. Fractions (7 ml each) are collected, and the presence of apo-ACP in the fractions is detected by SDS-PAGE as described above (16% tricine gels). The fractions containing apo-ACP are pooled and applied to an S-100 Sepharose gel filtration column (5×60 cm) equilibrated with 50 mM Tris-HCl, pH 7.0, 100 mM KCl. The column is eluted with the same buffer. Fractions (10 ml each) containing apo-ACP are collected, analyzed by electrospray mass spectrometry, and stored at −70° C. as described above.

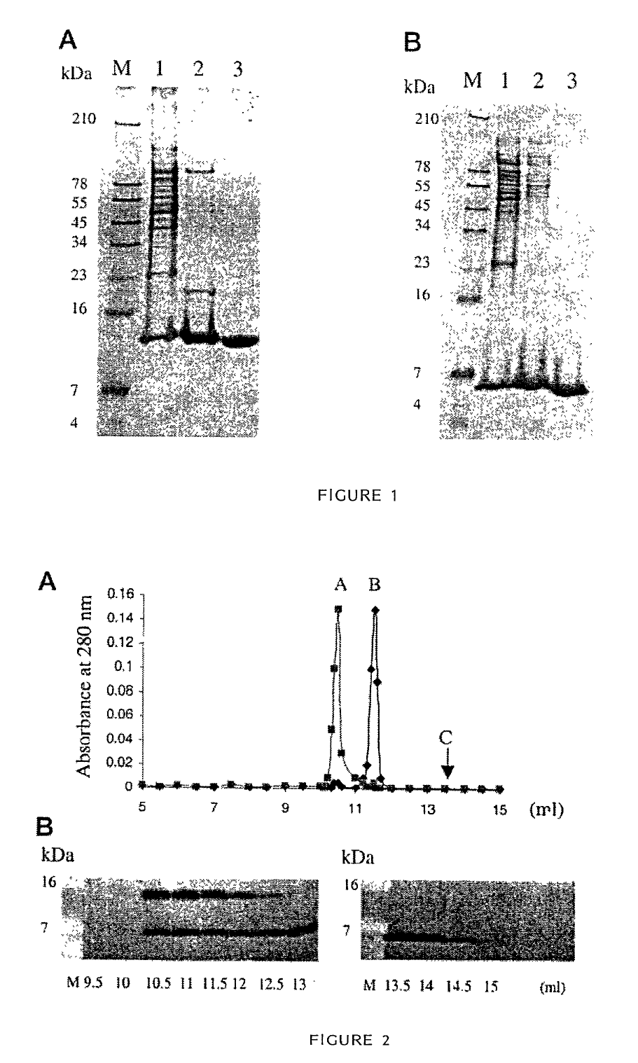

As shown in FIG. 1, AcpS is highly expressed in E. coli, and exhibits the predicted molecular weight of approximately 13,000 daltons. The overexpressed AcpS is purified to apparent homogeneity in two steps using Source S-cation-exchange and gel filtration column chromatography (FIG. 1).

To confirm the purified protein as AcpS, N-terminal sequencing and mass spectrometric analyses are performed. The first 9 amino acid residues of AcpS purified as described above are determined to be MIVGHGIDI (SEQ ID NO:6), a sequence that is identical to the predicted amino acid sequence for the protein encoded by the cloned acpS gene. This encoded protein is predicted to have a molecular weight of 13,388. Consistent with this predicted value, mass spectrometric analysis shows that purified AcpS has a molecular weight of 13,390. Thus, the purified protein is S. pneumoniae AcpS.

Analysis of AcpS by Gel Filtration Column Chromatography

To determine the native structure of AcpS, a purified AcpS preparation (375 μg) is applied to an S-75 Superdex gel filtration column (HR 1.0×30 cm), equilibrated with 50 mM Tris-HCl, pH 7.0, 50 mM KCl, 10 mM MgCl₂. The column is calibrated with protein molecular weight standards (Sigma). The effect of detergent or salt on the native structure of AcpS is analyzed by treating AcpS with 6 mM 3-cholamidopropyl-dimethylammonio-1-propane sulfonate/615 (CHAPS) or 50-500 mM KCl before and during column chromatography.

Sedimentation centrifugation analysis of AcpS is carried out using an XLA 10 ultracentrifuge (Beckman Instruments, Fullerton, Calif.). A purified AcpS preparation (adjusted to 0.2 and 0.4 mg/ml) is centrifuged at 16,000 rpm for 24 hr at 22° C. The absorbance at 280 nm as a function of radius after the system reaches equilibrium is analyzed using XL-A/XL-1, a non-linear least squares fit data analysis program. The partial specific volume of AcpS is calculated to be 0.721 ml/g based on its amino acid sequence. The molecular weight of AcpS is determined using a global fit of the two data sets collected with 0.2 and 0.4 mg/ml samples.

Cross-linking experiments are performed as follows. Purified AcpS and apo-ACP preparations (1 ml each) are dialyzed against 2 liters of 20 mM potassium phosphate buffer, pH 7.0, at 4° C. for 18 h. The dialyzed AcpS (163 μM) and apo-ACP (94 μM) preparations are mixed without or with 19.5 and 9.4 mM sulfo-EGS, respectively, and the mixtures are incubated at room temperature for 30 min. The reactions are stopped by the addition of 50 mM Tris-base, followed by incubation at room temperature for 30 min.

The resulting AcpS and apo-ACP preparations treated without or with the cross-linker (10 μl) are mixed with an equal volume of tricine sample buffer, and analyzed by SDS-PAGE (16% tricine gels).

To determine whether AcpS binds directly to apo-ACP or CoA in the absence of the other substrate, a purified AcpS preparation (27 μM) is first mixed with 10 mM MgCl2, and then either 100 μM apo-ACP or 50 μM CoA. The mixture is incubated at room temperature for 30 min and subjected to gel filtration column chromatography (S-75 Superdex) under the conditions described above. The fractions containing the AcpS-apo-ACP complex and unbound apo-ACP are analyzed by SDS-PAGE (16% tricine gels), SYPRO Orange staining, and mass spectrometry.

Enzyme Assay And Kinetics

Unless otherwise indicated, reaction mixtures contain 50 mM Tris-HCl, pH 7.0, 10 mM MgCl₂, 2.5-50 μM CoA, 0.25-6.0 μM purified apo-ACP of S. pneumoniae, and 3.7 nM purified AcpS of S. pneumoniae, and are incubated at 37° C. for 9 min. Reactions are stopped by the addition of 50 mM EDTA. The formation of holo-ACP is determined by an HPLC or a trichloroacetic acid (TCA) precipitation method (see below). An HPLC-based assay is adapted from the method described previously (Lambalot and Walsh, 1997). This assay monitors the conversion of apo-ACP to holo-ACP. Reaction mixtures (100 μl each) are injected into an analytical HPLC column (Vydac protein C4 reverse-phase; P. J. Cobert Associates, Inc., St. Louis, Mo.) equilibrated with 45% acetonitrile in 0.1% trifluoroacetic acid. The column is eluted with an 8 ml linear gradient of 45-80% acetonitrile. The column elution profiles are monitored at 220 nm. Under these conditions, holo-ACP migrates faster than apo-ACP. The amount of holo-ACP formed is estimated by comparing the peak area of the holo-ACP formed with those of both apo- and holo-ACP.

AcpS activity is also assayed by using a TCA precipitation method (Lambalot and Walsh, 1995). This method measures the incorporation of the 3H-labeled 4′-phospho-pantotheine group from [³H]CoA into apo-ACP. Reaction conditions are the same as those described for the HPLC assay, except that [³H]COA (specific activity, 1.5 Ci/mmol, NEN Life Science Products, Boston, Mass.) is used alone or in combination with CoA. Reactions are stopped by the addition of 0.9 ml of cold 10% TCA followed by 37 μg of bovine serum albumin (BSA). Precipitated protein is collected by centrifugation (a microfuge at 14,000 rpm) for 5 min and washed twice with ice-cold 10% TCA. The protein collected is resuspended in 150 μl of 1M Tris base and 0.1% Triton X-100. The resulting suspension (100 μl) is mixed with 2.5 ml of Ready Protein+scintillation fluid (Beckman, Fullerton, Calif.), and counted using an LS 60001C scintillation counter (Beckman).

To examine the substrate specificity of AcpS, a variety of CoA derivatives (acetyl-CoA, malonyl-CoA, acetoacetyl-CoA, desulfo-CoA, and dephospho-CoA) are tested. All reaction mixtures contain 1.5 μM apo-ACP, 20 μM CoA or CoA derivative, and 19 nM AcpS, and the formation of holo-ACP is determined by the HPLC method.

For the determination of Km and Vmax (kcat) of AcpS for apo-ACP, reaction mixtures (in quadruplicate) contain 20 μM CoA, 0.25 to 100 μM apo-ACP, and 3.7 nM. AcpS. The formation of holo-ACP is measured by the HPLC method or the TCA precipitation method. To determine Km for CoA, the reaction conditions are the same as those described above except that the concentration of apo-ACP is 2.0 μM, and the concentrations of CoA were 5 to 600 μM.

To analyze the kinetic mechanisms of AcpS, AcpS activity is measured at different concentrations of both substrates. Reaction mixtures (in triplicate) contain 2.5-40 μM CoA, 0.25-2.0 μM apo-ACP, and 3.7 nM AcpS. The formation of holo-ACP is analyzed by the HPLC assay.

To evaluate the inhibition of AcpS activity by 3′,5′-ADP with respect to CoA, reaction mixtures contain 1-60 μM 3′,5′-ADP, 2.5-60 μM CoA, 1.0 μM apo-ACP, and 3.7 nM AcpS. To evaluate the inhibition of AcpS activity by 3′,5′-ADP with respect to apo-ACP, reaction mixtures contain 1-60 μM 3′,5′-ADP, 0.5-6.0 μM apo-ACP, 20 μM CoA, and 3.7 nM AcpS. The formation of holo-ACP is analyzed by the HPLC method.

Identification and Organization of the acpS and acpP Genes of S. pneumoniae

To understand the function of AcpS in the biosynthesis of fatty acids in S. pneumoniae, cloning and expression of the acpS and acpP gene that encodes a substrate of AcpS are carried out. Both genes are identified from an S. pneumoniae data base (Baltz et al., 1998) using the E. coli acpS and acpP gene sequences as queries in the BLAST program (Altschul et al., 1990). The acpS gene, 369 bp long, encodes a protein consisting of 122 amino acid residues (SEQ ID NO: 1) with a predicted molecular mass of 13.7 kDa (GenBank™ accession number AF276617). The acpS gene appears to be organized into an operon with genes in the order aroG-aroF-acpS-alr-recG as there are long non-coding regions located upstream of aroG and downstream of recg. Thus, the acpS operon appears to consist of the genes that are required in aromatic amino acid biosynthesis (aroF and aroG encoding 3-deoxy-D-arabino-heptulosonate 7-phosphate synthases), cell wall biosynthesis (alr encoding D-alanine racemase), and DNA recombination (recG). In this regard, the genomic organization of acpS in S. pneumoniae is quite different from that of acpS in E. coli since acps in E. coli consists of an operon with its upstream pdxj gene that is required for vitamin B6 biosynthesis (Lam et al., 1992; Takiff et al., 1992).

The acpp gene, 234 bp long, encodes a protein consisting of 77 amino acid residues, with a predicted molecular weight of 8.7 kDa (GenBank™ accession number AF276618). The acpP gene appears to consist of an operon with the genes in the order hisC-unknown-plsX-acp. There are very long non-coding regions located in the upstream of hisC and downstream of acpP. Like the acpS operon, the genes in the acpp operon are also involved in different aspects of cellular metabolism such as histidine biosynthesis (his C encoding histidinol phosphate aminotransferase), lipid biosynthesis (plsx, required for the phenotype of plsB that encodes glycerol 3-phosphate acyltransferase, an enzyme required for lipid biosynthesis), and possibly others (unknown function gene). It is known that the acpP genes in Bacillus subtilis, E. coli, Pseudomonas aeruginosa, and Vibrio harveyi are organized into operons with other fatty acid biosynthetic genes (Kutchma et al., 1999; Morbidoni et al., 1996; Rawlings and Cronan, 1992; Shen and Byers, 1996). Thus, the operon organization of the acpp gene in S. pneumoniae is also different from those of the acpp genes in E. coli and other organisms (Kutchma et al., 1999; Morbidoni et al., 1996; Rawlings and Cronan, 1992; Shen and Byers, 1996). Finally, it is known that plsx and acpp, along with other fatty acid biosynthetic genes, are also located in the same operon in B. subtilis, E. coli, P. aeruginosa, and V. harveyi (Kutchma et al., 1999; Morbidoni et al., 1996; Rawlings and Cronan, 1992; Shen and Byers, 1996). This suggests that a genetic reorganization event might have occurred during evolution, which resulted in the formation of the complex operons that currently exist in organisms such as S. pneumoniae.

The subunits of S. pneumoniae AcpS and apo-ACP exhibit molecular weights virtually identical to those of E. coli AcpS and apo-ACP, respectively. Both proteins also share 38% identities with their counterparts in E. coli. The pI value of S. pneumoniae AcpS is estimated to be 6.5, which is much lower than 9.98, the pI value of E. coli AcpS (Lam et al., 1992; Takiff et al., 1992). Therefore, S. pneumoniae AcpS is significantly less basic than E. coli AcpS. Like other AcpS (Cronan and Rock, 1996; Rock and Cronan, 1996; Kutchma et al., 1999; Morbidoni et al., 1996; Rawlings and Cronan, 1992; Shen and Byers, 1996), S. pneumoniae apo-ACP is very acidic with a pI value of only 3.4.

Whether the S. pneumoniae acpS gene complements E. coli mutant strain HT253, which is defective in the production of AcpS (Takiff et al., 1992), is also investigated. HT253 contains a mini-TnlO insertion in the pdxJ gene, which is upstream of and forms an operon with acpS (Lam et al., 1992; Takiff et al., 1992). The mini-Tn10 carries two divergent tetracycline-inducible promoters (Takiff et al., 1992). In the absence of tetracycline, HT253 cannot grow on LB plates because the mini-TnlO insertion in pdxj blocks the transcription of the acpS gene. Thus, the growth of HT253 is tetracycline-dependent. When the acpS gene (pRBP123, acpS carried on pGEX-2T) is introduced into HT253, this mutant strain is able to grow on LB medium without the supplementation of tetracycline and IPTG. Apparently, the basal level expression of acpS without IPTG induction is sufficient for the complementation of HT253. This result clearly shows that the S. pneumoniae acpS gene complements the E. coli mutant deficient in the production of AcpS. Attempts to inactivate the acpS gene of S. pneumoniae through genetic insertional mutagenesis have failed (Baltz et al., 1997; P. Treadway, unpublished results), indicating that acpS is essential for growth. Since recG and air, down stream of acpS, are not essential genes (Kullik et al., 1998; Lloyd et al., 1996), we conclude that the acpS gene is essential for the growth of S. pneumoniae. Taken together, these results establish the identity of the gene as acpS, and that the function of acpS is essential for the growth of bacterial cells.

Expression, Puirifcation, and Identification of the AcpS and ACP of S. pneumoniae

The acpS and acpP genes identified are cloned into expression vectors and expressed in E. coli as described above. Both AcpS and apo-ACP are highly expressed in E. coli, and exhibit the molecular weights predicted (FIG. 1). The overexpressed AcpS is purified to apparent homogeneity in two steps (FIG. 1A) using Source S-cation-exchange and gel filtration column chromatography. The overexpressed S. pneumoniae apo-ACP is also purified to apparent homogeneity (FIG. 1B) in two steps using Source Q-anion exchange and gel filtration column chromatography.

To confirm the purified proteins as AcpS and ACP, the proteins can be subjected to N-terminal sequencing and mass spectrometric analyses. The first 9 amino acid residues of purified AcpS are determined to be MIVGHGIDI (SEQ ID NO:6), a sequence that is identical to the predicted amino acid sequence for the protein encoded by the cloned acpS gene (compare SEQ ID NO: 1). This encoded protein is predicted to have a molecular weight of 13,388. Consistent with this predicted value, mass spectrometric analysis shows that purified AcpS has a molecular weight of 13,390. Thus, the purified protein is S. pneumoniae AcpS.

N-terminal sequencing analysis also shows that purified apo-ACP exhibits the predicted amino acid sequence (data not shown). When subjected to mass spectrometric analysis, purified apo-ACP is found to exhibit two peaks. The major peak has a molecular mass of 8,834 Da (about 80% of the total protein), while the minor peak has a molecular mass of 8,861 Da (20%) that is 26 Da larger than that of the major species. The predicted molecular weight for S. pneumoniae apo-ACP is 8,706, in agreement with the results of mass spectrometric analysis. Mass spectrometric analysis further shows that both apo-AcpS are converted to holo-ACP upon their reaction with AcpS, since the molecular weights of both AcpS increase by 341 Da, corresponding to the molecular weight of the 4′-phospho-pantetheine group (data not shown). Finally, mass spectrometric analysis shows that holo-ACP is not detectable in the apo-ACP preparations (data not shown).

The mobilities of apo-ACP and holo-ACP can be examined by native gel electrophoresis followed by staining with SYPRO Orange. Holo-ACP migrates more slowly than apo-ACP (data not shown). The complete conversion of apo-ACP to holo-ACP is confirmed by the fact that the molecular weight of ACP increases from 8,834 Da (apo-ACP) to 9,174 Da (holo-ACP) upon treatment of apo-ACP with AcpS, CoA, and Mg⁺². Thus, unlike E. coli holo-ACP (5), S. pneumoniae holo-ACP migrates more slowly than apo-ACP.

Determination of the Native Structures of S. pneumoniae ACβS and ACP

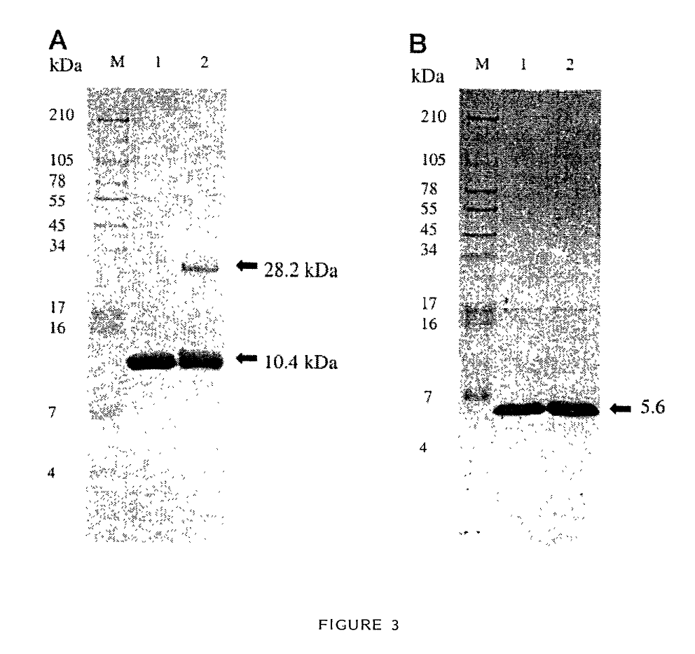

The molecular weight of native AcpS can be determined by subjecting a purified AcpS preparation to gel filtration column chromatography analysis. AcpS is eluted in the fractions corresponding to a molecular weight of 38 kDa (FIG. 2A, peak B). This result suggests that AcpS is a homotrimer with a predicted molecular mass of 41 kDa (GenBank™ accession number AF276617). To confirm this further, a purified AcpS preparation is subjected to sedimentation analysis. This analysis shows that purified AcpS has a molecular mass of 39 kDa, which is consistent with the gel filtration analysis. Finally, when a purified AcpS preparation is subjected to cross-linking followed by SDS-PAGE analysis, two protein bands are observed (FIG. 3). The two bands have molecular masses of 10.4 and 28.2 kDa, respectively, corresponding to the monomeric and trimeric forms of AcpS (FIG. 3, lane 2). Taken together, these results demonstrate that the AcpS of S. pneumoniae is a trimeric enzyme. The trimeric structure of AcpS appears to be stable as AcpS retains its native structure in the presence of 6 mM CHAPS or 50-500 mM KCl during gel filtration column chromatography (data not shown).

When apo-ACP is subjected to gel filtration column chromatography, it elutes in the fractions corresponding to a molecular mass of 17 kDa, indicating that apo-ACP may exist as a dimer (FIG. 2A). apo-ACP has been shown to behave abnormally on gel filtration columns due to its molecular asymmetry in shape (Cooper et al., 1987; Rock and Cronan, 1979). To examine further whether apo-ACP is a dimer, purified apo-ACP is subjected to cross-linking followed by SDS-PAGE analysis. Only one protein band is observed, having a molecular mass of 5.6 kDa (FIG. 3B). This result demonstrates that apo-ACP is a monomeric protein. The result of the gel filtration column analysis is consistent with the previously reported anomalous behavior of apo-ACP on gel filtration columns (Cooper et al., 1987; Rock and Cronan, 1979).

### EXAMPLE 2

### Kinetic Characterization of S. pneumoniae AcpS

To elucidate the reaction mechanism of AcpS, its substrate specificity and kinetics are examined. When assayed by the HPLC method, purified AcpS of S. pneumoniae exhibits optimal activity at 45-50° C. and pH 6.5, and is stable at 22-65° C. AcpS is able to utilize a number of CoA derivatives as substrates, and exhibits the following relative activities: 100 (CoA), 91 (acetyl-CoA), 76 (desulfo-CoA), 65 (acetoacetyl-CoA), 12 (malonyl-CoA), and 0 (dephospho-CoA). Thus, like E. coli AcpS and Bacillus subtilis Sfp protein, S. pneumoniae AcpS utilizes different CoA derivatives as substrates (Gehring et al., 1997; Quadri et al., 1998).

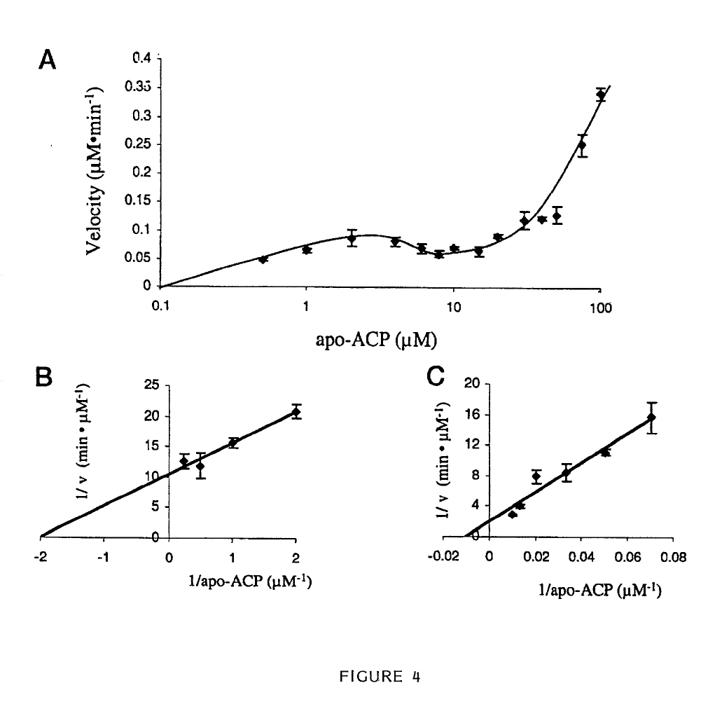

S. pneumoniae AcpS appears to exhibit Michaelis-Menten kinetics when assayed at various CoA concentrations and apo-ACP concentrations lower than 10 μM (FIG. 4A). AcpS activity increases in a dose-dependent manner at apo-ACP concentrations of 0.5-5 μM (FIG. 4A). When the concentration of apo-ACP approaches 10 μM, AcpS activity decreases (FIG. 4A). This result is consistent with the observation that apo-ACP is inhibitory to AcpS at higher concentrations (Elovson and Vagelos, 1968; Lambalot and Walsh, 1995; Flugel et al., 2000; Gehring et al., 1997). However, a further increase of apo-ACP concentrations (>10 μM) is accompanied by a significant increase in AcpS activity (FIG. 4A). As a result, two separate substrate saturation curves are obtained at low and high concentrations of apo-ACP (FIG. 4, B and C). Double reciprocal plot analyses indicate that AcpS has Km (for apo-ACP) values of 0.5±0.08 and 109±6.8 μM, and Vmax values of 2439±243 (kcat=1.7±0.17 s-1) and 13659±1290 (kcat=9.3±0.9 s-1) nmol/min/mg at the low and high concentrations of apo-ACP, respectively. Thus, at higher apo-ACP concentrations, the affinity of AcpS for apo-ACP is significantly decreased (approximately 200-fold), but its catalytic activity is significantly increased (5-fold). Together, these results indicate that the S. pneumoniae AcpS may be allosterically regulated by its substrate, apo-ACP.

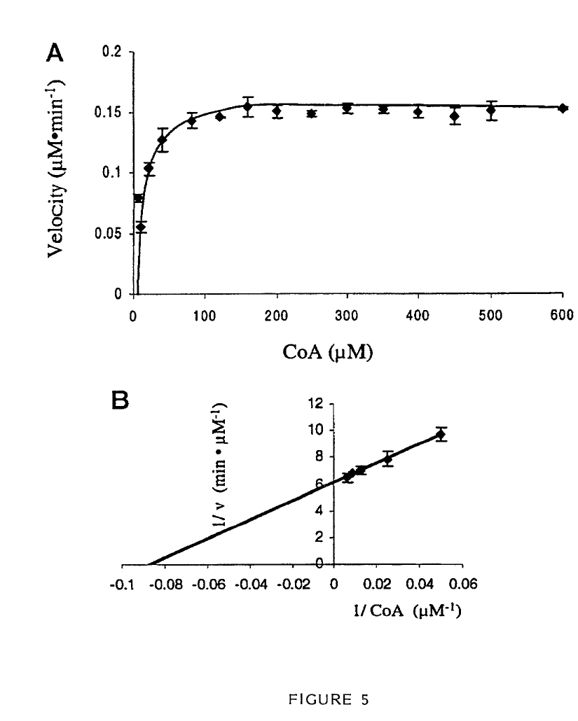

When a fixed apo-ACP concentration and various CoA concentrations are used, a hyperbolic substrate saturation curve is obtained for AcpS (FIG. 5A). The apparent Km and Vmax values of AcpS are determined to be 11.5±0.9 μM (for CoA) and 3976±73 nmol/min/mg (kcat=2.7±0.05 s-1), respectively (FIG. 5B). The kcat values determined for AcpS at low apo-ACP concentrations are thus in good agreement (1.7 versus 2.7 s⁻¹).

Since the TCA precipitation method has been often used for the assay of AcpS activity (Elovson and Vagelos, 1968; Lambalot and Walsh, 1995; Flugel et al., 2000), we also characterize the kinetic properties of the enzyme using this assay method. This assay utilizes [³H]CoA as a substrate for AcpS. The apparent Km values of the enzyme for apo-ACP and CoA are determined to be 1.3±0.7 and 7.1±0.4 μM, respectively. The Vmax (kcat) values determined are 4179±182 (2.8±0.04 s⁻¹) nmol/min/mg. Thus, the kinetic parameters determined by the TCA precipitation method are in general agreement with those obtained by the UPLC method. However, we did notice that the TCA precipitation method tends to generate variations significantly higher than those of the HPLC method, especially when apo-ACP is below 1 μM.

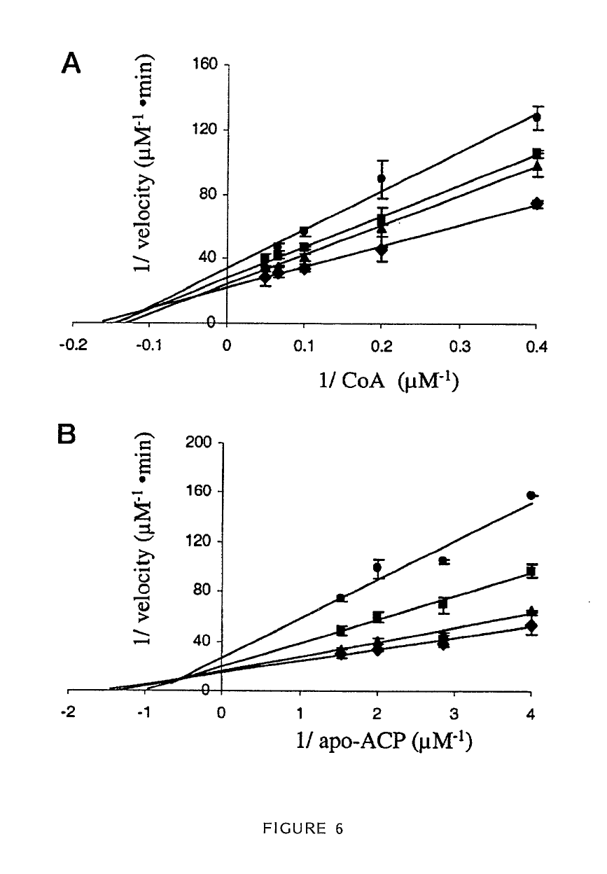

Although E. coli AcpS has been extensively studied (Elovson and Vagelos, 1968; Lambalot and Walsh, 1995; Flugel et al., 2000; Lambalot et al., 1996), the kinetic mechanism of this enzyme is unknown. To elucidate further the kinetic mechanism of S. pneumoniae AcpS, double reciprocal plots of the initial velocities of the enzyme at fixed concentrations of one substrate versus various concentrations of the other substrate (Copeland, 1996) are analyzed. This analysis yields Km and Vmax values that are similar to those determined before (data not shown). As shown in FIG. 6A, the double reciprocal plots of the initial velocities of AcpS obtained at the various CoA and fixed apo-ACP concentrations yields an intersecting pattern. The same pattern is obtained when various concentrations of apo-ACP and fixed concentrations of CoA are used (FIG. 6B). Taken together, these results suggest that AcpS proceeds by a random or compulsory ordered bi bi type, but not a ping-pong (double displacement) type, of reaction mechanism (Copeland, 1996).

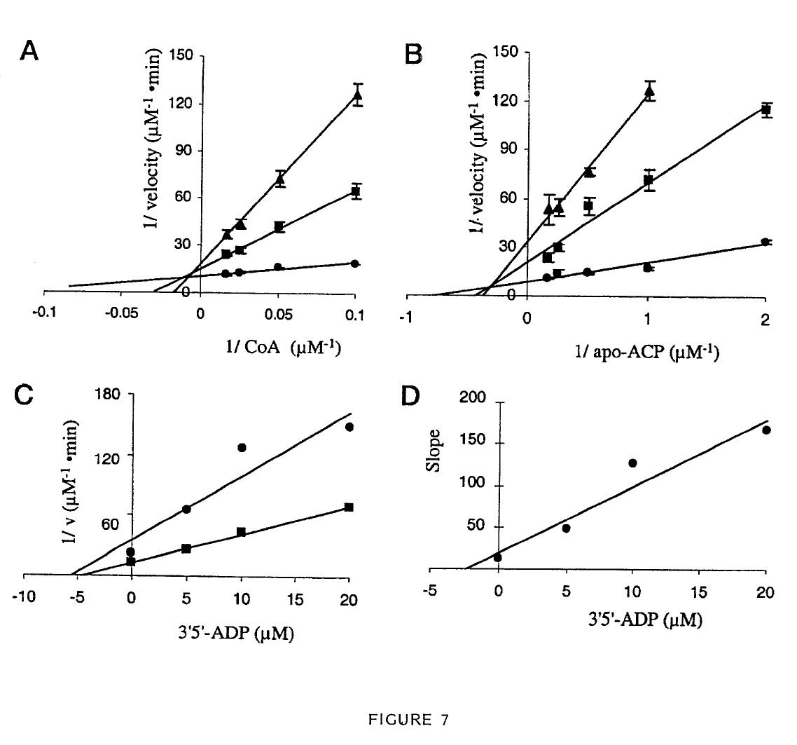

To differentiate these two possible reaction mechanisms, the kinetics of product inhibition are analyzed. AcpS activity is examined in the presence of 3′,5′-ADP. As shown in FIG. 7A, when various CoA concentrations are used, the double reciprocal plots yield a simple competitive pattern with a Ki of 6.0 μM (FIG. 7C). However, when various apo-ACP concentrations are used, the double reciprocal plots yield a linear-mixed pattern with a Ki of 2.5 μM (FIG. 7, A and D). Since the patterns of inhibition with respect to CoA and apo-ACP are competitive and mixed, respectively, these results suggest that apo-ACP is probably the first substrate to bind to the enzyme, which is followed by CoA (Copeland, 1996).

### EXAMPLE 3

### Binding of apo-ACP and CoA to AcpS

The order of substrate binding to AcpS can be analyzed by determining the binding of CoA and apo-ACP to purified AcpS by gel filtration column chromatography, mass spectrometry, or filter binding assays. If CoA binds to AcpS first and forms an enzyme-substrate complex that is required for the next reaction with apo-ACP, then a stable enzyme-substrate complex should be detectable. When a mixture of CoA and purified AcpS incubated at room temperature for 30 min is subjected to gel filtration column chromatographic and mass spectrometric analyses, the CoA is not detectable in the fractions containing purified AcpS (data not shown). Thus, CoA does not appear to bind to AcpS in the absence of apo-ACP. To examine further the binding of CoA to AcpS, a mixture of purified AcpS and [³H]CoA incubated under the same conditions is subjected to a filter binding assay. Under these conditions, [³H]CoA does not appear to bind to AcpS as the radioactivity of [3H]CoA is not detectable after washing (data not shown). Consistent with the foregoing evidence, CoA does not appear to bind to AcpS in the absence of apo-ACP.

Whether apo-ACP binds to AcpS in the absence of CoA, a mixture of AcpS and apo-ACP (apo-ACP/AcpS=5:1) can be subjected to gel filtration column chromatography and analysis of the column fractions by SDS-PAGE. Two protein peaks are observed (FIG. 2A). The leading peak (peak A) has a molecular mass of approximately 53 kDa as judged by gel filtration analysis (FIG. 2A). Since AcpS exists as a trimer with a molecular mass of approximately 41 kDa, this leading peak probably represents a complex between apo-ACP and AcpS (FIG. 2A). Consistent with the formation of the AcpS-apo-ACP complex, the presence of apo-ACP is also detected in the fractions containing purified AcpS (FIG. 2B). Together, these results demonstrate that apo-ACP can bind to AcpS in the absence of CoA. In summary, the analysis of the initial velocities of AcpS obtained at fixed concentrations of one substrate and various concentrations of another reveals that catalysis by AcpS probably proceeds by a random or ordered compulsory bi bi reaction mechanism because an intersecting pattern was obtained regardless of which substrate (CoA or apo-ACP) was the fixed one or the varied one (Copeland, 1996) (FIG. 6). The inhibition kinetics of 3′,5′-ADP, one of the reaction products, indicates that AcpS catalysis appears to proceed by an ordered reaction mechanism, with the initial formation of an AcpS-apo-ACP intermediate and the subsequent transfer of 4′-phosphopantetheine from CoA onto apo-ACP. The mode of inhibition by 3′,5′-ADP with respect to CoA is competitive when apo-ACP is the fixed substrate and CoA is the varied substrate (FIG. 7). The competitive inhibition with respect to CoA indicates that CoA only binds to the enzyme-apo-ACP intermediate. The mode of inhibition by 3′,5′-ADP with respect to apo-ACP is mixed, i.e., a combination of competitive and noncompetitive inhibition when apo-ACP is the varied substrate and CoA is the fixed substrate (FIG. 7B). The mixed type of inhibition by 3′,5′-ADP with respect to apo-ACP suggests that 3′,5′-ADP binds to the free enzyme and the enzyme-apo-ACP intermediate. Thus, inhibition is competitive with respect to apo-ACP when 3′,5′-ADP binds to the free enzyme, and noncompetitive with respect to apo-ACP when 3′,5′-ADP binds to the enzyme-apo-ACP intermediate. This proposed reaction mechanism for AcpS is consistent with the results of the substrate-binding experiments. Under the conditions tested, apo-ACP binds tightly to AcpS in the absence of CoA, but CoA fails to bind in the absence of apo-ACP. Taken together, these results suggest that the reaction mechanism of AcpS is ordered rather than random, and that the formation of the enzyme-apo-ACP intermediate occurs prior to the transfer of 4′-phosphopantethine from CoA onto apo-ACP.

### EXAMPLE 4

### Crystallization of AcpS; Data Collection, Structure Solution, and Refinement

Production and Purification of Selenomethionine-Containing AcpS

Selenomethionine is incorporated into AcpS using the method of Doublie, 1997, which is based on inhibition of the methionine biosynthetic pathway. One ml of E. coli BL21 (pLysS) cells containing pETI la that carries the acpS gene of S. pneumoniae from an overnight culture in LB medium is centrifuged, and resuspended in 1 ml of M9 minimal medium (Sambrook et al., 1989) supplemented with 100 μg/ml ampicillin and 4 g/L glucose. This 1 ml suspension is added to 1 liter of the same medium pre-warmed at 33° C. Cells are grown at 33° C. with shaking at 250 rpm to an optical density of 0.5-0.6 at 590 nm (mid-log phase) before addition of amino acids as follows: lysine, phenylalanine, and threonine at 100 mg/L; isoleucine, leucine, and valine at 50 mg/L; and L-selenomethionine at 60 mg/L. Expression is induced with 1 mM IPTG 15 min after adding amino acids, and continued for 18 hours. The cells are harvested, and AcpS is purified as described above, the only modifications being the addition of 1 mM dithiothreitol (DTT) to the purification buffers to prevent oxidation of the seleno-methionine groups, and Mono Q chromatography.

The fractions from the gel filtration FPLC step containing AcpS (verified by 16% Tricine SDS-PAGE) are pooled and applied directly to a Mono Q ion exchange FPLC column (1 ml bed volume) (Pharmacia) equilibrated in 50 mM Tris-HCl, pH 7.0. The column is washed for 10 bed volumes (10 mls) in this buffer, and then a 0M to 1M KCl gradient is applied. The column is run at 1.0 ml/min, detection is at 280 nm, and 1 ml fractions are collected. The fractions are analyzed by 16% tricine SDS-PAGE. Fractions containing AcpS are pooled, dialyzed against 50 mM Tris-HCl, pH 7.0, 140 mM KCl, 10 mM MgCl₂, analyzed for protein concentration using Bradford Protein assay (BioRad Laboratories) and bovine serum albumin as a standard. The resulting protein solution is then used for crystallography.

Crystallization of AcpS

Diffraction-quality crystals are grown by the vapor diffusion technique at 294K. The protein is concentrated using a Centricon filter (molecular weight cutoff=10 kDa) to 8 mg/ml in a solution of 10 mM MgCl₂, 14 mM KCl, and 20 mM Tris-HCl buffer at pH 7.1. A 4 μl (1:1, v/v, protein/reservoir solution) drop is equilibrated in a 500 μl solution containing 8-15% PEG 4000, 200 mM ammonium sulfate, and 100 mM citrate buffer at pH 4.5. Crystals of AcpS are obtained after 4 to 5 days at room temperature. Crystals belong to orthorhombic space group P2₁2₁2₁ (unit cell parameters a=49.8 Å, b=59.6 Å, c=114.7 Å) (Native 1; Tables 2 and 3). Crystallization conditions similar to those described above also yield crystals that belong to monoclinic space group C2 (unit cell parameters a=120.2 Å, b=62.3 Å, c=51.7 Å, β=98.7°) for apo-AcpS (Native 2; Tables 2 and 4) and 3′,5′-ADP complex (Tables 2 and 5). Both crystal forms have a homotrimeric molecule per asymmetric unit, with a Vm value (Matthews, 1968) of 2.08 Å³/Da, which corresponds to a solvent content of approximately 41% in both cases. CoA, in 2-3 fold of excess of the protein, is used as a starting material for co-crystallization in the 3′,5′-ADP complex.

Data Collection, Structure Solution, and Refinement

The diffraction data are collected using a MarCCD detector on IMCA (Industrial Macromolecular Crystallography Association) beam line ID-17 at the APS (Advanced Photon Source, Argonne National Laboratories) at 100K, using 15-20% glycerol as a cryoprotectant. The diffraction data are reduced using DENZO (HKL2000) (Otwinowski and Minor 1997), and the intensities are scaled with SCALEPACK (Collaborative Computing Project, No. 4, 1994). Most calculations are performed with the CCP4 suite of programs (Collaborative Computing Project, No. 4, 1994). Multiple anomalous dispersion (MAD) data at three wavelengths around the selenium K-shell edge are collected from a single crystal (selenomethionine-substituted protein) belonging to the P2₁2₁2₁ space group at 2.8 Å resolution using the inverse beam strategy (Hendrickson et al., 1991). Location of the Se sites and phasing are performed using SOLVE (Terwilliger and Berendzen, 1999), resulting in a figure of merit of 0.64 for the data in the resolution range 20-2.8 Å. Experimental phases are subsequently modified by the application of solvent flattening and 3-fold NCS averaging using the program DM (Collaborative Computing Project, No. 4, 1994). The experimental map allows tracing of 115 amino acid residues, excluding the three-residue loop corresponding to residues Gly69-Lys73, and the four C-terminal residues for each monomer molecule of the homotrimer. This model is refined against data between 20 and 2.4 Å using a maximum likelihood algorithm as incorporated in the program CNX2000 (Badger et al., 1999) (R_{work}=0.236, R_{free}=0.293) (Brünger, 1992). Subsequently, the coordinates of the trimer are used as a search model in molecular replacement (AmoRe) (Navaza, 1994) for the C2 space group crystals. This structure is refined to an R_{work} of 0.208 (R_{free}=0.247) against the data in the resolution range of 20-2.0 Å. The AcpS/3′,5′-ADP complex structure is refined to an R_{work} of 24.1% (R_{free}=27.1%) for the 20-1.9 Å resolution range. The program suite QUANTA 98 (Molecular Simulation Inc., San Diego, Calif.) is used for visual inspection and manual corrections between rounds of refinement. An analysis of the geometry shows that all parameters are within the values expected for a model at this resolution. All residues are found in the most favorable and additionally allowed regions of a Ramachandran plot for all three crystal structures. The last four residues in each AcpS protomer and residues Gly69-Lys7l of a disordered surface loop for one of the subunits are not defined in the electron density even after crystallographic refinement, and for this reason are not included in the final model. The overall average temperature factor of the structures are in good agreement with that calculated from the Wilson plot.

Coordinates are deposited in the PDB under entry codes 1FTE, 1FTF, and 1FTH.

### EXAMPLE 5

### Overall Structure of Apo-AcpS

Data are collected from crystals and the structure of AcpS solved by the multiple anomalous dispersion method (MAD) (Hendrickson et al., 1991) using selenomethionine-substituted protein and exploiting non-crystallographic 3-fold averaging. The atomic coordinates of native AcpS (Native 1 and Native 2) are shown in Tables 3 and 4, respectively. The crystallographic data collection statistics and refinement parameters are summarized in Table 2.

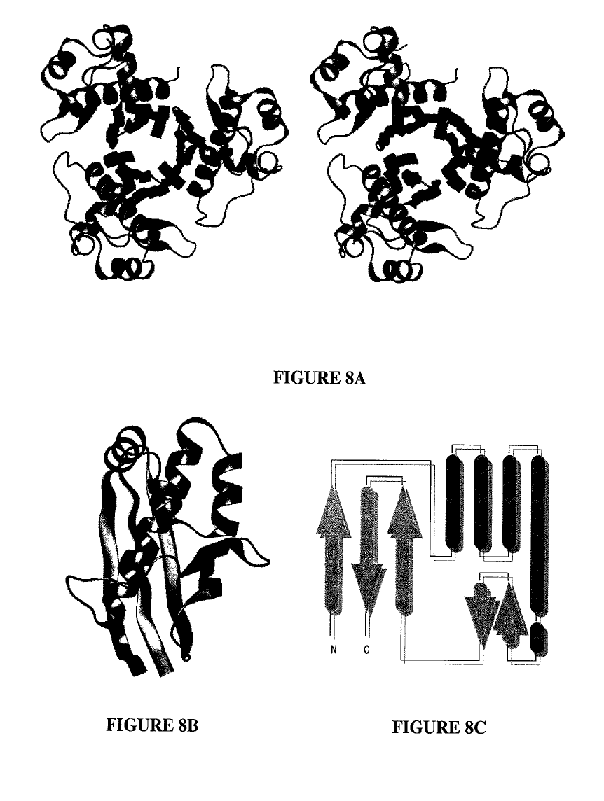

The structure of AcpS reveals that it assembles as a tightly packed homotrimer. The overall view of the AcpS molecule is shown in FIG. 8A. The AcpS monomer has an elongated elliptical shape with approximate dimensions of 30×35×45 Å (FIG. 8B). The Richardson topology diagram of secondary structural elements is shown in FIG. 8C. The location of these secondary structure elements within the protein sequence is given in Table lA. The AcpS structure has an α/β fold. A topology search using the SCOP program (Murzin et al 1995) does not reveal any significant similarity with AcpS. The AcpS protomer is characterized primarily by three structural motifs. The first is a classical three-stranded anti-parallel β-sheet formed by strands β1, β5, and β4. A long α-helix packs diagonally against the β-sheet together with α-helixes α1, α2, α3, and α4 of the anti-parallel four helical bundle, which represents the second structural motif. The third feature consists of a long extended loop with a two-strand anti-parallel β-sheet β2 and β3). These structural motifs are organized in such a way that the long helix α4 runs through the whole structure and is surrounded by the other structural elements.

The side chains of each helix in the four-helical bundle are arranged so that hydrophobic side chains are buried between the helices and form a hydrophobic core comprised of Ile 17, Ala 20, Val 21 (α1); Phe 27, Ala 28, Val 31, Leu 32 (α2); Met 37, Phe 40 (α3); Ile 49, Leu 52, Trp 56 (α4). Side chains of the residues Phe 75 and Ile 70 (α5) also participate in the formation of this hydrophobic cluster. Another hydrophobic cluster is formed by the long α-helix α4, and the three- and two-stranded β-sheets, which contain side chains of the residues Phe 62, Met 66 (α4); Phe 90 (β3); Phe 95, Ile99 (β4).

The final refined structure of AcpS contains 115 out of 122 amino acids. No electron density is observed for the first two N-terminal and final four C-terminal amino acids. The three protomers are related by a non-crystallographic 3-fold pseudo symmetry axis. The three-stranded anti-parallel β-sheets of each AcpS protomer are arranged together in a barrel-like structure in the homotrimer molecule, forming a long, mostly hydrophobic tunnel that runs through the whole structure. The active sites are formed at the intermolecular interface of the homotrimer (FIG. 8B) such that two protomers contribute to the formation of each active site. An active site pocket is formed by residues of helix α4, 1-strands β4 and β5, and loop β2-β3 of one protomer, and the opposite side of helix α4, β-stranded β5 and loop α4-α5 of the second protomer molecule. Since the AcpS active site is created by the interface between two monomers, which are oriented by their homotrimer architecture, the trimeric structure of AcpS appears to be essential for activity. This is consistent with the results of dynamic light scattering (Protein Solution Inc., Charlottesville, Va.), gel filtration column chromatography, and sedimentation analysis of the purified AcpS, which demonstrate that AcpS is a homotrimer (note Example 1 and McAllister et al., 2000). Finally, the native structure reveals that only two out of three active sites are occupied by sulfate ions that are present during the crystallization of AcpS. The sulfate ions are found to be present in the vicinity of His105, Asp10, and Lys64, which corresponds to the α-phosphate of CoA (see below).

### EXAMPLE 5

### Co-Crystallization of Purified AcpS with Coenzyme A; Overall Structure of the AcpS/3′,5′-ADP Complex

To help identify the potential active site of AcpS for a structure-based design effort, purified AcpS is co-crystallized with CoA, a substrate of AcpS. Surprisingly, however, the purified AcpS is co-crystallized with 3′,5′-ADP, a product of the AcpS reaction. The fact that only the 3′,5′-ADP moiety exhibits a well defined electron density clearly demonstrates that 3′,5′-ADP, rather than CoA, co-crystallizes with AcpS. Consistent with the native structure of AcpS, the structure of the AcpS/3′,5′-ADP complex shows that AcpS is a trimeric enzyme. In addition, only two out of three active sites are occupied by 3′,5′-ADP in this case, rather than sulfate ions as in the case of the native structure, thus indicating that the binding of the 3′,5′-ADP molecules to AcpS competes with that of the sulfate ions. These results are also in good agreement with those of gel filtration and SDS-PAGE analysis (supra, and McAllister et al., 2000) which demonstrate that ACP is bound to AcpS in a 2:3 ratio. Finally, similar to that of the native structure, the unoccupied active site of the complex structure also exhibits no defined electron density for residues Ile70-Leu73 of the α4-α5 loop. Thus, the complex structure of AcpS suggests that the active form of the enzyme is trimeric.

The atomic coordinates of the AcpS/3′,5′-ADP complex are shown in Table 5; the active site of the AcpS/3′,5′-ADP complex is shown in FIG. 9. The 3′,5′-ADP binding site is characterized by the following structural elements. The adenine base fits in between loop β2-β3 (Gly86, Ala87, and Pro88) and loop α4-α5 (Lys64, Gly67, and Thr68) from another protomer of the AcpS trimer. An amino group of the adenine ring is in a favorable position to form a hydrogen bond with a carbonyl oxygen of Arg85 and Thr68. The ribose moiety is bound to the adenine ring with an anti-glycosidic torsion angle. The ribose is present in a 3′-endo conformation with the axial orientation of the 2′-hydroxyl and the equatorial orientation of the 3′-phosphate group. The 3′-phosphate portion of the ligand has an interaction with loop β2-β3, α4, and β4, all of which belong to the first protomer. The 3′-phosphate is surrounded by a negatively charged cluster formed by Arg39, Arg47, and Arg55. The 5′ α-phosphate is packed against β1 and α4 of the second protomer, and has hydrogen bonds with Lys64, Ser104, and His105.

In summary, AcpS, which catalyzes the transfer of 4′-phosphopantetheine from CoA to apo-ACP to form holo-ACP along with the production of 3′,5′-ADP, is an attractive target for the development of antibacterial drugs. The structure of AcpS reveals an α/β fold, and demonstrates that the trimeric structure of the enzyme appears to be essential for the AcpS activity. These results represent the first structural determination of the interaction between the AcpS enzyme and its product, 3′,5′-ADP. These data provide a starting point for structure-based diagnostics, and drug design efforts that will enable identification of novel AcpS inhibitors with potent antibacterial activity useful in treating bacterial infections.

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

### Literature Cited

Ahamed, J., Gangopadhyay, J., Sinha, A. K. (1999) Antimicrob. Agents. Chemother. 43, 2333-2334.

Altschul, S. F., Gish, W., Miller, W., Myers, E. W. and Lipman, D. J. (1990) J. Mol. Biol. 215, 403-410.

Appelt (1993) Perspectives in Drug Discovery and Design 1:23-48.

Ausubel et al., eds. (1987 and supplements) Current Protocols in Molecular Biology, John Wiley & Sons, N.Y.

Badger J., Kumar R. A., Yip P., Szalma S. (1999) X-PLOR Proteins 35, 25-33.

Baltz et al. (1997) Abstracts of the 37th ICAAC, p. 391(S-⁴6), Insterscience Conference on Antirnicrobial Agents and Chemotherapy, Toronto, Canada.

Baltz, R. H., Norris, F. H., et al. (1998) Microbial Drug Resistance 4, 1-9.

Blattner, F. R., Plunkett G., III, Bloch, C. A., et al., (1997) Science 277, 1453-1462.

Bradford, M. M. (1976) Anal. Biochem. 72:248-254.

Brünger A. T. (1992) Nature 355, 472-474.

Bugg et al. (1993) Scientific American, December:92-98.

Carreras, C. W., A. M. Gehring, C. T. Walsh, and C. Khosla. (1997) Biochemistry, 36:11757-11761.

Cohen, M. L. (1992) Science 257, 1050-1055.

Cole, S. T., Brosch, R., et al. (1998) Nature 393, 537-544.

Collaborative Computing Project, No. 4. (1994) Acta Crysta. D50, 760-763.

Cooper, C. L., Boyce, S. G., and Lueking, D. R. (1987) Biochemistry 26, 2740-2746.

Copeland, R. A. (1996) Enzymes: A Practical Introduction to Structure, Mechanism, and Data Analysis, Wiley-VCH, Inc., New York, N.Y.

Cronan, J. E., Jr., and Rock, C. O. (1996) in Escherichia coli and Salmonella: Cellular and Molecular Biology (Neidhardt, F. C., Curtiss, R., III, Ingramham, J. L., Lin, E. C. C., Low, K. B., Magasanik, B., Reznikoff, W. S., Riley, M., Schaechter, M., and Umbarger, H. E., eds) pp. 612-636, ASM Press, Washington, D.C.

Crosby, J., D. H. Sherman, M. J. Bibb, W. P. Revill, D. A. Hopwood, T. J. Simpson. (1995) Biochim. Biophys. Acta. 1251:32-42.

Cunningham and Wells (1989) Science 244:1081-1085.

Cwirla et al. (1990) Proc. Natl. Acad. Sci. USA 87:6378-6382.

Davies, J. (1994) Science 264, 375-382.

Devlin et al. (1990) Science 249:404-406.

de Vos et al. (1992) Science 255:306-312.

Doublié (1997) Preparation of selenomethionyl proteins for phase determination, Methods in Enzymology 276: 523-530.

Dunbrack et al. (1997) Folding & Design 2:R27-42.

Elovson, J., P. R. Vagelos. (1968) J. Biol. Chem. 243, 3603-3611.

Erickson (1993) Perspectives in Drug Discovery and Design 1: 109-128).

Flugel, R. S., Y. Hwangbo, R. H. Lambalot, J. E. Cronan, Jr., and C. T. Walsh. (2000) J. Biol. Chem. 275:959-968.

Gehring, A. M., R. H. Lambalot, K. W. Vogel, D. G. Drueckhammer, and C. T. Walsh. (1997) Chem. Biol. 4:17-24.

Heath, R. J., and Rock, C. O. (1996) J. Biol. Chem. 271, 1833-1836.

Hendrickson W. A. (1991) Science 254, 51-58.

Himmelreich, R., Hilbert, H., Plagens, H., Pirkl, E., Li, B. -C., and Herrmann, R. (1996) Nucleic Acid Res. 24, 4420-4449.

Jackowski S, and Rock, C. O. (1981) J. Bacteriol. 148,926-932.

Jackowski, S., and Rock, C. O. (1983). J. Biol. Chem. 258, 15186-15191.

Jackowski S., and Rock, C. O. (1984) J. Bacteriol. 158, 115-120.

Jackowski, S., and Rock, C. O. (1986) J. Bacteriol. 166, 866-871.

Jones et al. (1991) Acta Cryst. A47:110-119.

Kalman, S., Mitchell, W., Marathe, R., Lammel, C., Fan, J., Hyman, R. W., Olinger, L., Grimwood, J., Davis, R. W., and Stephens, R. S. (1999) Nature Genet. 21, 385-389.

Keating, D. H., Carey, M. R., and Cronan, J. E., Jr. (1995) J. Biol. Chem. 270, 22229-22235.

Kubitschek, H. E., and Friske, J. A. (1986) J. Bacteriol. 168, 1466-1467.

Kullik et al. (1998) Gene 219:9-17.

Kuns, F., Ogasawara, N., Moszer, I., et al. (1997) Nature 390, 249-256.

Kutchma, A. J., Hoang, T. T., and Schwezer, H. P. (1999) J. Bacteriol. 181:5498-5504.

Kyte and Doolittle (1982) J. Mol. Biol. 157: 105-132.

Laemmli, U. K. (1970) Nature 227:680-685.

Lam , H-M, E. Tancula, W. B. Dempsey, M. E. Winkler. (1992) J. Bacteriol. 174, 1554-1567.

Lam et al. (1994) Science 263:380-384.

Lambalot, R. H., C. T. Walsh. (1995) J. Biol. Chem. 270, 24658-24661.

Lambalot, R. H., and Walsh, C. T. (1997) Methods Enzymol. 279, 254-262.

Lambalot, R. H., A. M. Gehring, R. S. Flugel, P. Zuber, M. LaCelle, M. A. Marahiel, R.

Reid, C. Khosla, C. T. Walsh. (1996) Curr. Biol. 3, 923-936.

Lloyd et al. (1996) in Escherichia coli and Salmonella: Cellular and Molecular Biology, F. C. Neidhardt et al., eds., pp. 2236-2255, American Society for Microbiology Press, Washington, D.C.

Magnuson, K., S. Jackowski, C. O. Rock, J. E. Cronan, Jr. (1993) Microbiol. Rev. 57, 522-542.

Majerus, P. W., Alberts, A. W., and Vagelos, P. R. (1965) Proc. Natl. Acad. Sci. USA 20 53:410-417.

Matthews, B. W. (1968) J. Mol. Biol 33, 491-497.

McAllister, K. A., Peery, R. B., Meier, T. I., Fischl, A. S., and Zhao, G. (2000) J. Biol. Chem. 275, 30864-30872.

Morbidoni, H. R., Mendoza, D. D., and Cronan, J. E., Jr. (1996) J. Bacteriol. 178, 4794-4800.

Murzin A. G., Brenner S. E., Hubbard T., Chothia C. (1995) J. Mol. Biol. 247, 536-540

Navaza J. (1994) Acta Cryst. A50, 157-163.

Neu, H. C. (1992) Science 257:1064-1073.

Otwinowski Z, Minor W. In: Methods in Enzymology (Carter C. W. Jr and Sweet R. M., Eds., Academic Press) 276, 307-326.

Parris K. D., Lin L., Tam A., Mathew R., Hixon J., Stahl M, Fritz C. C., Seehra J. Somers W. S. (2000) Structure with Folding and Design, 8:883-895.

Quadri, L. E. N., Weinreb, P. H., Lie, M., Nakano, M. M., Zuber, P., and Walsh, C. T. (1998). Biochemistry 37, 1585-1595.

Rawlings, M., and Cronan, J. E., Jr. (1992) J. Biol. Chem. 267, 5751-5754.

Reuter K., Mofid M. R., Marahiel M. A., Ficner R. (1999) The EMBO J. 23, 6823-6831.

Rock, C. O., and Cronan, J. E., Jr. (1979) J. Biol. Chem. 254, 9778-9785.

Rock, C. O., and Cronan, J. E., Jr. (1996) Bioch. Biophys. Acta. 1302, 1-16.

Sambrook et al. (1989) Molecular Cloning, A Laboratory Manual, Second Edition, Cold Spring Harbor Laboratory Press, Cold Spring Harbor, N.Y.

Scott et al. (1990) Science 249:386-390.

Shen, Z., and Byers, D. M. (1996) J. Bacteriol. 178, 571-573.

Smith, et al. (1992) J. Mol. Biol. 224:899-904 (1992).

Smith, S. (1994) FASEB 8, 1248-1259.

Spratt, B. G. (1994a) Science 264, 388-393.

Spratt B. G. (1994b) “Resistance to β-lactam antibiotics” in Ghuysen J. M. and Hakenbeck R., eds., Bacterial Cell Wall, Elsevier, Amsterdam, The Netherlands, pp. 517-534.

Takiff, J. E., T. Baker, T. Copeland, S-M. Chen, D. L. Court. (1992) J. Bacteriol. 174, 1544-1553.

Terwilliger, T C & Berendzen, J. (1999) Acta Crystallogr. D 55, 849-861.

Thomson, K. S., Sanders, C. C. (1998) Antimicrob. Agents. Chemother. 42, 179-187.

Tomasz, A., Munoz, R. (1995) Microb. Drug. Resis. 1, 103-109.

Tomb, J. -F., White, O.,l Kerlavage, A. R., et al. (1997) Nature 388, 539-547.

Tropf, S., Revill, W. P., Bibb, M. J., Howood, D. A., and Schweizer, M. (1998) Chem. Biol. 5:135-146.

Vallari, D. S., Jackowski, S., and Rock, C. O. (1987) J. Biol. Chem. 262, 2468-2471.

Vallari, D. S., and Jackowski, S. (1988) J. Bacteriol. 170, 3961-3966.

West et al. (1995) TIPS 16:67-74.

Wlodawer et al. (1993) Ann. Rev. Biochem. 62:543-585.

Zhao, G., Meier, T. I., Peery, R. B., Matsushima, P., and Skatrud, P. L. (1999) Microbiology 145:791-800.

Zhou, P., Florova, G., and Reynolds, K. A. (1999) Chem. Biol. 6:577-584.

TABLE 1A

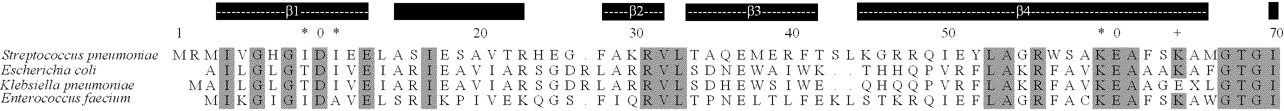

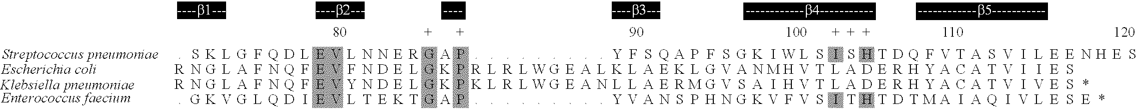

TABLE 1B

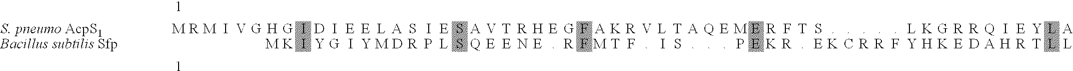

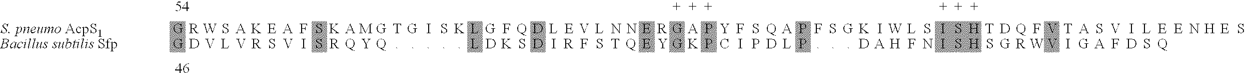

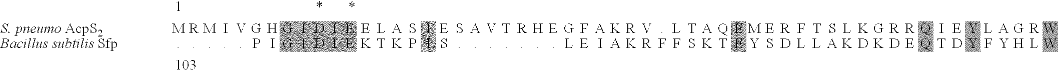

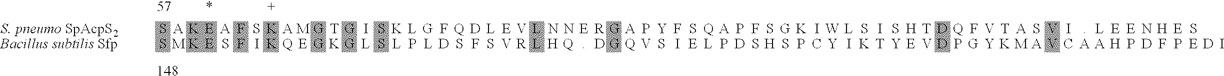

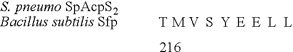

TABLE 2
Crystallographic data

| | | | | | |
|---|---|---|---|---|---|
| Inflection, L1 | Peak, L2 | Remote, L3 | Native 1 | ^{&}Native 2 | ^{&}3′5′-ADP |

Data collection

| | | | | | | |
|---|---|---|---|---|---|---|
| Energy, eV | 12,655.75 | 12,659.28 | 12,700.00 | 12,398.00 | 12,398.00 | 12,398.00 |
| Wavelength λ, Å | 0.979537 | 0.979400 | 0.976259 | 1.00000 | 1.00000 | 1.00000 |
| Number of observations | 210,097 | 205,948 | 211,823 | 336,325 | 423,425 | 304,946 |
| Number of reflections | 54,462 | 55,080 | 54,821 | 59,335 | 126,451 | 162,841 |
| Number of unique reflections | 8,450 | 8,508 | 8,398 | 14,842 | 20,366 | 29,541 |
| Average redundancy | 6.4 | 6.5 | 6.5 | 4.0 | 6.2 | 5.5 |
| Completeness, % | | | | | | |
| All reflections | ^{a}95/90 | 95/88 | 94/88 | 96/84 | 94/89 | 100/99 |
| Reflections with I > 3σ | 94/85 | 94/86 | 94/84 | 81/57 | 79/53 | 80/44 |
| <I/σ> overall | 40/14 | 41/19 | 40/18 | 25.6/4.5 | 26.5/3.0 | 31.1/3.8 |
| *R_{merge}, % | 5.6/9.2 | 5.2/8.8 | 4.7/8.4 | 4.9/23.0 | 6.0/30.1 | 5.3/26.8 |
| Resolution, Å | 2.8 | 2.8 | 2.8 | 2.4 | 2.0 | 1.9 |

Refinement

| | | | |
|---|---|---|---|
| Protein/Water | 2,696/128 | 2,730/125 | 2,717/177 |
| Deviation form ideal geometry | | | |
| bond length, Å/bond angles, ° | 0.007/1.169 | 0.005/1.057 | 0.010/1.146 |
| dihedrals/imprpers, ° | 25.956/0.588 | 23.123/0.610 | 22.247/0.770 |
| Ramachandran plot statistics | | | |
| Residues in most favorite/allowed regions, % | 92.0/8.0 | 91.2/9.0 | 92.4/7.6 |
| †R_{work}/^{¶}R_{free}, % | 23.6/29.3 | 20.8/24.7 | 24.1/27.1 |

^{a}Data after slash sign correspond to outer shell
*R_{merge} = Σ|I − <I>|/ΣI, where I is the intensity of an individual measurement, and <I> is the mean intensity of this reflection
^{¶}R_{free} is the cross-validation R-value calculated for 5% of the reflections omitted from the refinement.
†R_{work} = Σ|Fₒ − F_{c}|/Σ|Fₒ|, where |Fₒ| and |F_{c}| are the observed and calculated structure factor amplitudes respectively.
^{&}Space group for these data sets is monoclinic C2, the rest of the data sets belong to orthorombic P2₁2₁2₁ space group. Both of them contain one homotrimer per the asymmetric unit

TABLE 3
REMARK coordinates from restrained individual B-factor refinement
REMARK refinement resolution: 500.0-2.40 A

| | |
|---|---|
| REMARK starting | r = 0.2339 free_r = 0.2899 |
| REMARK final | r = 0.2336 free_r = 0.2950 |

| | |
|---|---|
| REMARK B rmsd for bonded mainchain atoms = 0.775 | target = 1.5 |
| REMARK B rmsd for bonded sidechain atoms = 0.798 | target = 2.0 |
| REMARK B rmsd for angle mainchain atoms = 1.417 | target = 2.0 |
| REMARK B rmsd for angle sidechain atoms = 1.333 | target = 2.5 |

REMARK wa = 3.19192
REMARK rweight = 0.44902
REMARK target = mlf steps = 30
REMARK sg = P2(1)2(1)2(l) a = 49.824 b = 59.561 c = 113.738 alpha = 90

beta = 90 gamma = 90

REMARK parameter file 1: CNX_TOPPAR:protein_rep.param
REMARK parameter file 2: CNX_TOPPAR:water_rep.param
REMARK parameter file 3: CNX_TOPPAR:ion.param
REMARK parameter file 4: so4/so4.param
REMARK molecular structure file: generate.psf
REMARK input coordinates: minimize.pdb
REMARK reflection file = x-ray_data.cv
REMARK ncs = none
REMARK B-correction resolution: 6.0-2.40
REMARK initial B-factor correction applied to fobs:
REMARK B11 = 5.613 B22 = 4.955 B33 = −10.569
REMARK B12 = 0.000 B13 = 0.000 B23 = 0.000
REMARK B-factor correction applied to coordinate array B: 0.171
REMARK bulk solvent: (Mask) density level = 0.356315 e/A\hat{ }3, B-factor = 44.7059 A\hat{ }2
REMARK reflections with |Fobs|/sigma_F < 0.0 rejected
REMARK reflections with |Fobs| > 10000 * rms(Fobs) rejected

| | | |
|---|---|---|
| REMARK theoretical total number of refl. in resol. range: | 13818 | (100.0%) |
| REMARK number of unobserved reflections (no entry or \|F\| = 0): | 537 | ( 3.9%) |
| REMARK number of reflections rejected: | 0 | ( 0.0%) |
| REMARK total number of reflections used: | 13281 | ( 96.1%) |
| REMARK number of reflections in working set: | 12603 | ( 91.2%) |
| REMARK number of reflections in test set: | 678 | ( 4.9%) |

REMARK FILENAME = “acps_native 1.pdb”
REMARK Written by CNX VERSION: 2000

| | | | | | | | | | | | |
|---|---|---|---|---|---|---|---|---|---|---|---|
| ATOM | 1 | CB | MET | A1003 | 6.910 | 33.184 | 39.062 | 1.00 | 31.51 | C | |
| ATOM | 2 | CG | MET | A1003 | 5.553 | 33.663 | 38.598 | 1.00 | 33.32 | C | |
| ATOM | 3 | SD | MET | A1003 | 4.818 | 34.801 | 39.816 | 1.00 | 36.27 | S | |
| ATOM | 4 | CE | MET | A1003 | 5.820 | 36.290 | 39.557 | 1.00 | 35.34 | C | |
| ATOM | 5 | C | MET | A1003 | 7.655 | 32.931 | 36.686 | 1.00 | 29.56 | C | |
| ATOM | 6 | O | MET | A1003 | 8.286 | 33.972 | 36.449 | 1.00 | 29.16 | O | |
| ATOM | 7 | N | MET | A1003 | 9.076 | 32.131 | 38.552 | 1.00 | 30.78 | N | |
| ATOM | 8 | CA | MET | A1003 | 7.675 | 32.305 | 38.074 | 1.00 | 30.32 | C | |
| ATOM | 9 | N | ILE | A1004 | 6.935 | 22.292 | 35.768 | 1.00 | 28.43 | N | |
| ATOM | 10 | CA | ILE | A1004 | 6.826 | 32.791 | 34.401 | 1.00 | 27.65 | C | |
| ATOM | 11 | CB | ILE | A1004 | 6.307 | 31.701 | 33.452 | 1.00 | 28.01 | C | |
| ATOM | 12 | CG2 | ILE | A1004 | 6.103 | 32.287 | 32.051 | 1.00 | 27.49 | C | |
| ATOM | 13 | CG1 | ILE | A1004 | 7.294 | 30.525 | 33.430 | 1.00 | 27.59 | C | |
| ATOM | 14 | CD1 | ILE | A1004 | 6.890 | 29.404 | 32.482 | 1.00 | 28.22 | C | |
| ATOM | 15 | C | ILE | A1004 | 5.885 | 33.992 | 34.322 | 1.00 | 26.94 | C | |
| ATOM | 16 | O | ILE | A1004 | 4.741 | 33.920 | 34.765 | 1.00 | 26.43 | O | |
| ATOM | 17 | N | VAL | A1005 | 6.380 | 35.088 | 33.750 | 1.00 | 25.94 | N | |
| ATOM | 18 | CA | VAL | A1005 | 5.595 | 36.306 | 33.617 | 1.00 | 25.33 | C | |
| ATOM | 19 | CB | VAL | A1005 | 6.211 | 37.446 | 34.445 | 1.00 | 25.03 | C | |
| ATOM | 20 | CG1 | VAL | A1005 | 6.389 | 36.995 | 35.882 | 1.00 | 25.00 | C | |
| ATOM | 21 | CG2 | VAL | A1005 | 7.535 | 37.878 | 33.846 | 1.00 | 24.19 | C | |
| ATOM | 22 | C | VAL | A1005 | 5.432 | 36.796 | 32.173 | 1.00 | 25.45 | C | |
| ATOM | 23 | O | VAL | A1005 | 4.982 | 37.920 | 31.949 | 1.00 | 25.63 | O | |
| ATOM | 24 | N | GLY | A1006 | 5.794 | 35.964 | 31.197 | 1.00 | 25.11 | N | |
| ATOM | 25 | CA | GLY | A1006 | 5.659 | 36.371 | 29.809 | 1.00 | 24.32 | C | |
| ATOM | 26 | C | GLY | A1006 | 6.306 | 35.452 | 28.786 | 1.00 | 23.99 | C | |
| ATOM | 27 | O | GLY | A1006 | 7.413 | 34.947 | 28.996 | 1.00 | 24.03 | O | |
| ATOM | 28 | N | HIS | A1007 | 5.613 | 35.232 | 27.671 | 1.00 | 23.11 | N | |
| ATOM | 29 | CA | HIS | A1007 | 6.132 | 34.386 | 26.603 | 1.00 | 22.22 | C | |
| ATOM | 30 | CB | HIS | A1007 | 5.633 | 32.943 | 26.789 | 1.00 | 22.37 | C | |
| ATOM | 31 | CG | HIS | A1007 | 6.046 | 32.010 | 25.693 | 1.00 | 22.55 | C | |
| ATOM | 32 | CD2 | HIS | A1007 | 5.331 | 31.123 | 24.961 | 1.00 | 22.89 | C | |
| ATOM | 33 | ND1 | HIS | A1007 | 7.342 | 31.933 | 25.226 | 1.00 | 22.71 | N | |
| ATOM | 34 | CE1 | HIS | A1007 | 7.405 | 31.044 | 24.251 | 1.00 | 22.72 | C | |
| ATOM | 35 | NE2 | HIS | A1007 | 6.198 | 30.537 | 24.070 | 1.00 | 22.98 | N | |
| ATOM | 36 | C | HIS | A1007 | 5.718 | 34.925 | 25.234 | 1.00 | 21.79 | C | |
| ATOM | 37 | O | HIS | A1007 | 4.553 | 35.230 | 25.007 | 1.00 | 21.69 | O | |
| ATOM | 38 | N | GLY | A1005 | 6.682 | 35.054 | 24.325 | 1.00 | 21.62 | N | |
| ATOM | 39 | CA | GLY | A1008 | 6.380 | 35.549 | 22.994 | 1.00 | 20.43 | C | |
| ATOM | 40 | C | GLY | A1005 | 7.237 | 34.888 | 21.932 | 1.00 | 20.87 | C | |
| ATOM | 41 | O | GLY | A1008 | 8.396 | 34.554 | 22.171 | 1.00 | 20.58 | O | |
| ATOM | 42 | N | ILE | A1009 | 6.655 | 34.663 | 20.760 | 1.00 | 20.92 | N | |
| ATOM | 43 | CA | ILE | A1009 | 7.389 | 34.077 | 19.652 | 1.00 | 21.06 | C | |
| ATOM | 44 | CB | ILE | A1009 | 6.911 | 32.638 | 19.304 | 1.00 | 21.27 | C | |
| ATOM | 45 | CG2 | ILE | A1009 | 7.140 | 31.699 | 20.485 | 1.00 | 21.48 | C | |
| ATOM | 46 | CG1 | ILE | A1009 | 5.438 | 32.654 | 18.892 | 1.00 | 21.01 | C | |
| ATOM | 47 | CD1 | ILE | A1009 | 4.967 | 31.340 | 18.285 | 1.00 | 20.98 | C | |
| ATOM | 48 | C | ILE | A1009 | 7.184 | 34.951 | 18.420 | 1.00 | 21.74 | C | |
| ATOM | 49 | O | ILE | A1009 | 6.345 | 35.846 | 18.404 | 1.00 | 20.69 | O | |
| ATOM | 50 | N | ASP | A1010 | 7.971 | 34.687 | 17.388 | 1.00 | 22.85 | N | |
| ATOM | 51 | CA | ASP | A1010 | 7.857 | 35.416 | 16.147 | 1.00 | 23.91 | C | |
| ATOM | 52 | CB | ASP | A1010 | 8.456 | 36.813 | 16.273 | 1.00 | 23.93 | C | |
| ATOM | 53 | CG | ASP | A1010 | 8.300 | 37.622 | 14.998 | 1.00 | 24.40 | C | |
| ATOM | 54 | OD1 | ASP | A1010 | 9.104 | 37.423 | 14.050 | 1.00 | 24.86 | O | |
| ATOM | 55 | OD2 | ASP | A1010 | 7.357 | 38.442 | 14.939 | 1.00 | 24.36 | O | |
| ATOM | 56 | C | ASP | A1010 | 8.553 | 34.658 | 15.041 | 1.00 | 24.83 | C | |
| ATOM | 57 | O | ASP | A1010 | 9.586 | 34.037 | 15.262 | 1.00 | 25.16 | O | |
| ATOM | 58 | N | ILE | A1011 | 7.959 | 34.687 | 13.852 | 1.00 | 25.82 | N | |
| ATOM | 59 | CA | ILE | A1011 | 8.541 | 34.021 | 12.697 | 1.00 | 27.04 | C | |
| ATOM | 60 | CB | ILE | A1011 | 7.713 | 32.780 | 12.258 | 1.00 | 26.91 | C | |
| ATOM | 61 | CG2 | ILE | A1011 | 6.274 | 33.168 | 11.940 | 1.00 | 26.65 | C | |
| ATOM | 62 | CG1 | ILE | A1011 | 8.378 | 32.126 | 11.048 | 1.00 | 27.05 | C | |
| ATOM | 63 | CD1 | ILE | A1011 | 7.818 | 30.757 | 10.708 | 1.00 | 27.44 | C | |
| ATOM | 64 | C | ILE | A1011 | 8.586 | 35.051 | 11.580 | 1.00 | 27.96 | C | |
| ATOM | 65 | O | ILE | A1011 | 7.652 | 35.832 | 11.404 | 1.00 | 28.18 | O | |
| ATOM | 66 | N | GLU | A1012 | 9.688 | 35.070 | 10.845 | 1.00 | 28.86 | N | |
| ATOM | 67 | CA | GLU | A1012 | 9.841 | 36.023 | 9.762 | 1.00 | 30.03 | C | |
| ATOM | 68 | CB | GLU | A1012 | 10.772 | 37.161 | 10.186 | 1.00 | 31.54 | C | |
| ATOM | 69 | CG | GLU | A1012 | 10.071 | 38.292 | 10.895 | 1.00 | 33.83 | C | |
| ATOM | 70 | CD | GLU | A1012 | 9.006 | 38.913 | 10.026 | 1.00 | 35.22 | C | |
| ATOM | 71 | OE1 | GLU | A1012 | 9.352 | 39.404 | 8.927 | 1.00 | 36.13 | O | |
| ATOM | 72 | OE2 | GLU | A1012 | 7.825 | 38.903 | 10.438 | 1.00 | 36.10 | O | |
| ATOM | 73 | C | GLU | A1012 | 10.375 | 35.414 | 8.487 | 1.00 | 29.94 | C | |
| ATOM | 74 | O | GLU | A1012 | 11.219 | 34.526 | 8.513 | 1.00 | 29.44 | O | |
| ATOM | 75 | N | GLU | A1013 | 9.864 | 35.901 | 7.367 | 1.00 | 30.50 | N | |
| ATOM | 76 | CA | GLU | A1013 | 10.331 | 35.444 | 6.069 | 1.00 | 30.96 | C | |
| ATOM | 77 | CE | GLU | A1013 | 9.236 | 35.587 | 5.012 | 1.00 | 31.74 | C | |
| ATOM | 78 | CG | GLD | A1013 | 9.629 | 34.951 | 3.693 | 1.00 | 33.86 | C | |
| ATOM | 79 | CD | GLU | A1013 | 8.621 | 35.179 | 2.582 | 1.00 | 35.01 | C | |
| ATOM | 80 | OE1 | GLU | A1013 | 8.792 | 34.550 | 1.515 | 1.00 | 36.28 | O | |
| ATOM | 81 | OE2 | GLU | A1013 | 7.675 | 35.980 | 2.762 | 1.00 | 35.21 | O | |
| ATOM | 82 | C | GLU | A1013 | 11.498 | 36.372 | 5.735 | 1.00 | 30.42 | C | |
| ATOM | 83 | O | GLU | A1013 | 11.348 | 37.594 | 5.750 | 1.00 | 30.06 | O | |
| ATOM | 84 | N | LEU | A1014 | 12.662 | 35.800 | 5.457 | 1.00 | 30.14 | N | |
| ATOM | 85 | CA | LEU | A1014 | 13.824 | 36.617 | 5.140 | 1.00 | 30.32 | C | |
| ATOM | 86 | CB | LEU | A1014 | 15.022 | 35.722 | 4.814 | 1.00 | 30.23 | C | |
| ATOM | 87 | CG | LEU | A1014 | 15.552 | 34.904 | 6.000 | 1.00 | 30.34 | C | |
| ATOM | 88 | CD1 | LEU | A1014 | 16.618 | 33.929 | 5.533 | 1.00 | 30.59 | C | |
| ATOM | 89 | CD2 | LEU | A1014 | 16.117 | 35.835 | 7.053 | 1.00 | 30.41 | C | |
| ATOM | 90 | C | LEU | A1014 | 13.526 | 37.563 | 3.977 | 1.00 | 30.65 | C | |
| ATOM | 91 | O | LEU | A1014 | 13.877 | 38.743 | 4.023 | 1.00 | 30.37 | O | |
| ATOM | 92 | N | ALA | A1015 | 12.857 | 37.048 | 2.949 | 1.00 | 30.96 | N | |
| ATOM | 93 | CA | ALA | A1015 | 12.519 | 37.855 | 1.784 | 1.00 | 31.57 | C | |
| ATOM | 94 | CB | ALA | A1015 | 11.683 | 37.038 | 0.798 | 1.00 | 30.98 | C | |
| ATOM | 95 | C | ALA | A1015 | 11.780 | 39.131 | 2.183 | 1.00 | 31.93 | C | |
| ATOM | 96 | O | ALA | A1015 | 11.947 | 40.170 | 1.544 | 1.00 | 31.75 | O | |
| ATOM | 97 | N | SER | A1016 | 10.972 | 39.055 | 3.240 | 1.00 | 32.40 | N | |
| ATOM | 98 | CA | SER | A1016 | 10.227 | 40.224 | 3.714 | 1.00 | 33.29 | C | |
| ATOM | 99 | CB | SER | A1016 | 9.264 | 39.844 | 4.847 | 1.00 | 33.61 | C | |
| ATOM | 100 | OG | SER | A1016 | 8.316 | 38.883 | 4.428 | 1.00 | 34.88 | O | |
| ATOM | 101 | C | SER | A1016 | 11.194 | 41.279 | 4.235 | 1.00 | 33.73 | C | |
| ATOM | 102 | O | SER | A1016 | 11.059 | 42.464 | 3.937 | 1.00 | 33.37 | O | |
| ATOM | 103 | N | ILE | A1017 | 12.156 | 40.828 | 5.035 | 1.00 | 34.48 | N | |
| ATOM | 104 | CA | ILE | A1017 | 13.167 | 41.698 | 5.615 | 1.00 | 35.17 | C | |
| ATOM | 105 | CB | ILE | A1017 | 14.041 | 40.922 | 6.641 | 1.00 | 34.97 | C | |
| ATOM | 106 | CG2 | ILE | A1017 | 15.151 | 41.814 | 7.173 | 1.00 | 34.62 | C | |
| ATOM | 107 | CG1 | ILE | A1017 | 13.172 | 40.436 | 7.805 | 1.00 | 35.03 | C | |
| ATOM | 108 | CD1 | ILE | A1017 | 12.550 | 41.555 | 8.623 | 1.00 | 34.83 | C | |
| ATOM | 109 | C | ILE | A1017 | 14.064 | 42.266 | 4.516 | 1.00 | 35.70 | C | |
| ATOM | 110 | O | ILE | A1017 | 14.435 | 43.439 | 4.555 | 1.00 | 35.49 | O | |
| ATOM | 111 | N | GLU | A1018 | 14.414 | 41.431 | 3.542 | 1.00 | 36.59 | N | |
| ATOM | 112 | CA | GLU | A1018 | 15.260 | 41.872 | 2.439 | 1.00 | 38.02 | C | |
| ATOM | 113 | CB | GLU | A1018 | 15.595 | 40.703 | 1.508 | 1.00 | 39.36 | C | |
| ATOM | 114 | CG | GLU | A1018 | 16.768 | 40.993 | 0.578 | 1.00 | 42.30 | C | |
| ATOM | 115 | CD | GLU | A1018 | 16.928 | 39.967 | −0.532 | 1.00 | 44.15 | C | |
| ATOM | 116 | OE1 | GLU | A1018 | 16.923 | 38.748 | −0.239 | 1.00 | 45.23 | O | |
| ATOM | 117 | OE2 | GLU | A1018 | 17.069 | 40.387 | −1.703 | 1.00 | 45.34 | O | |
| ATOM | 118 | C | GLU | A1018 | 14.480 | 42.925 | 1.658 | 1.00 | 38.06 | C | |
| ATOM | 119 | O | GLU | A1018 | 15.026 | 43.936 | 1.212 | 1.00 | 37.81 | O | |
| ATOM | 120 | N | SER | A1019 | 13.187 | 42.668 | 1.509 | 1.00 | 37.94 | N | |
| ATOM | 121 | CA | SER | A1019 | 12.297 | 43.561 | 0.799 | 1.00 | 37.74 | C | |
| ATOM | 122 | CB | SER | A1019 | 10.921 | 42.906 | 0.680 | 1.00 | 37.89 | C | |
| ATOM | 123 | OG | SER | A1019 | 9.994 | 43.764 | 0.044 | 1.00 | 38.96 | O | |
| ATOM | 124 | C | SER | A1019 | 12.182 | 44.916 | 1.505 | 1.00 | 37.72 | C | |
| ATOM | 125 | O | SER | A1019 | 12.074 | 45.957 | 0.850 | 1.00 | 37.89 | O | |
| ATOM | 126 | N | ALA | A1020 | 12.207 | 44.902 | 2.836 | 1.00 | 37.14 | N | |
| ATOM | 127 | CA | ALA | A1020 | 12.097 | 46.129 | 3.615 | 1.00 | 37.01 | C | |
| ATOM | 128 | CB | ALA | A1020 | 11.773 | 45.800 | 5.063 | 1.00 | 36.69 | C | |
| ATOM | 129 | C | ALA | A1020 | 13.369 | 46.973 | 3.542 | 1.00 | 37.03 | C | |
| ATOM | 130 | O | ALA | A1020 | 13.309 | 48.201 | 3.538 | 1.00 | 36.27 | O | |
| ATOM | 131 | N | VAL | A1021 | 14.519 | 46.309 | 3.497 | 1.00 | 37.47 | N | |
| ATOM | 132 | CA | VAL | A1021 | 15.797 | 47.007 | 3.415 | 1.00 | 38.34 | C | |
| ATOM | 133 | CB | VAL | A1021 | 16.979 | 46.047 | 3.651 | 1.00 | 37.99 | C | |
| ATOM | 134 | CG1 | VAL | A1021 | 18.282 | 46.740 | 3.316 | 1.00 | 38.47 | C | |
| ATOM | 135 | CG2 | VAL | A1021 | 16.990 | 45.585 | 5.093 | 1.00 | 38.14 | C | |
| ATOM | 136 | C | VAL | A1021 | 15.937 | 47.605 | 2.025 | 1.00 | 38.86 | C | |
| ATOM | 137 | O | VAL | A1021 | 16.345 | 48.749 | 1.855 | 1.00 | 38.89 | O | |
| ATOM | 138 | N | THR | A1022 | 15.586 | 46.810 | 1.029 | 1.00 | 39.74 | N | |
| ATOM | 139 | CA | THR | A1022 | 15.670 | 47.244 | −0.350 | 1.00 | 40.51 | C | |
| ATOM | 140 | CB | THR | A1022 | 15.220 | 46.117 | −1.300 | 1.00 | 40.54 | C | |
| ATOM | 141 | OG1 | THR | A1022 | 15.975 | 44.930 | −1.019 | 1.00 | 40.47 | O | |
| ATOM | 142 | CG2 | THR | A1022 | 15.442 | 46.525 | −2.753 | 1.00 | 41.07 | C | |
| ATOM | 143 | C | THR | A1022 | 14.795 | 48.472 | −0.579 | 1.00 | 40.85 | C | |
| ATOM | 144 | O | THR | A1022 | 15.227 | 49.433 | −1.204 | 1.00 | 41.01 | O | |
| ATOM | 145 | N | ARG | A1023 | 13.577 | 48.442 | −0.048 | 1.00 | 41.28 | N | |
| ATOM | 146 | CA | ARG | A1023 | 12.626 | 49.541 | −0.215 | 1.00 | 41.51 | C | |
| ATOM | 147 | CB | ARG | A1023 | 11.198 | 49.003 | −0.083 | 1.00 | 41.55 | C | |
| ATOM | 148 | CG | ARG | A1023 | 10.804 | 48.042 | −1.201 | 1.00 | 42.10 | C | |
| ATOM | 149 | CD | ARG | A1023 | 9.460 | 47.375 | −0.938 | 1.00 | 42.16 | C | |
| ATOM | 150 | NE | ARG | A1023 | 8.439 | 48.328 | −0.514 | 0.05 | 42.12 | N | |
| ATOM | 151 | CZ | ARG | A1023 | 7.158 | 48.021 | −0.344 | 0.05 | 42.14 | C | |
| ATOM | 152 | NH1 | ARG | A1023 | 6.734 | 46.785 | −0.567 | 0.05 | 42.17 | N | |
| ATOM | 153 | NH2 | ARG | A1023 | 6.300 | 48.947 | 0.061 | 0.05 | 42.16 | N | |
| ATOM | 154 | C | ARG | A1023 | 12.798 | 50.747 | 0.710 | 1.00 | 41.61 | C | |
| ATOM | 155 | O | ARG | A1023 | 12.396 | 51.851 | 0.363 | 1.00 | 41.34 | O | |
| ATOM | 156 | N | HIS | A1024 | 13.395 | 50.544 | 1.878 | 1.00 | 42.27 | N | |
| ATOM | 157 | CA | HIS | A1024 | 13.583 | 51.636 | 2.835 | 1.00 | 42.70 | C | |
| ATOM | 158 | CB | HIS | A1024 | 12.658 | 51.445 | 4.040 | 0.05 | 42.61 | C | |
| ATOM | 159 | CG | HIS | A1024 | 11.202 | 51.471 | 3.693 | 0.05 | 42.54 | C | |
| ATOM | 160 | CD2 | HIS | A1024 | 10.233 | 50.537 | 3.841 | 0.05 | 42.53 | C | |
| ATOM | 161 | ND1 | HIS | A1024 | 10.592 | 52.567 | 3.121 | 0.05 | 42.51 | N | |
| ATOM | 162 | CE1 | HIS | A1024 | 9.311 | 52.307 | 2.932 | 0.05 | 42.49 | C | |
| ATOM | 163 | NE2 | HIS | A1024 | 9.067 | 51.081 | 3.360 | 0.05 | 42.48 | N | |
| ATOM | 164 | C | HIS | A1024 | 15.028 | 51.727 | 3.307 | 1.00 | 43.14 | C | |
| ATOM | 165 | O | HIS | A1024 | 15.483 | 50.930 | 4.131 | 1.00 | 43.24 | O | |
| ATOM | 166 | N | GLU | A1025 | 15.732 | 52.722 | 2.782 | 1.00 | 43.47 | N | |
| ATOM | 167 | CA | GLU | A1025 | 17.139 | 52.963 | 3.089 | 1.00 | 43.74 | C | |
| ATOM | 168 | CB | GLU | A1025 | 17.596 | 54.249 | 2.393 | 1.00 | 44.50 | C | |
| ATOM | 169 | CG | GLU | A1025 | 17.396 | 54.230 | 0.888 | 1.00 | 45.87 | C | |
| ATOM | 170 | CD | GLU | A1025 | 18.031 | 55.420 | 0.208 | 1.00 | 46.86 | C | |
| ATOM | 171 | OE1 | GLU | A1025 | 19.267 | 55.571 | 0.320 | 1.00 | 47.25 | O | |
| ATOM | 172 | OE2 | GLU | A1025 | 17.294 | 56.201 | −0.434 | 1.00 | 47.49 | O | |
| ATOM | 173 | C | GLU | A1025 | 17.539 | 53.030 | 4.565 | 1.00 | 43.28 | C | |
| ATOM | 174 | O | GLU | A1025 | 18.655 | 52.645 | 4.912 | 1.00 | 43.56 | O | |
| ATOM | 175 | N | GLY | A1026 | 16.649 | 53.523 | 5.424 | 1.00 | 42.64 | N | |
| ATOM | 176 | CA | GLY | A1026 | 16.972 | 53.631 | 6.838 | 1.00 | 41.75 | C | |
| ATOM | 177 | C | GLY | A1026 | 16.244 | 52.645 | 7.731 | 1.00 | 41.40 | C | |
| ATOM | 178 | O | GLY | A1026 | 16.091 | 52.877 | 8.931 | 1.00 | 41.35 | O | |
| ATOM | 179 | N | PHE | A1027 | 15.801 | 51.537 | 7.144 | 1.00 | 40.74 | N | |
| ATOM | 180 | CA | PHE | A1027 | 15.077 | 50.500 | 7.871 | 1.00 | 39.69 | C | |
| ATOM | 181 | CB | PHE | A1027 | 14.695 | 49.376 | 6.906 | 1.00 | 39.76 | C | |
| ATOM | 182 | CG | PHE | A1027 | 13.976 | 48.233 | 7.558 | 1.00 | 40.05 | C | |
| ATOM | 183 | CD1 | PHE | A1027 | 12.654 | 48.369 | 7.978 | 1.00 | 40.03 | C | |
| ATOM | 184 | CD2 | PHE | A1027 | 14.618 | 47.012 | 7.748 | 1.00 | 39.83 | C | |
| ATOM | 185 | CE1 | PHE | A1027 | 11.982 | 47.301 | 8.576 | 1.00 | 39.93 | C | |
| ATOM | 186 | CE2 | PHE | A1027 | 13.955 | 45.944 | 8.344 | 1.00 | 39.71 | C | |
| ATOM | 187 | CZ | PHE | A1027 | 12.636 | 46.089 | 8.757 | 1.00 | 39.70 | C | |
| ATOM | 188 | C | PHE | A1027 | 15.900 | 49.920 | 9.020 | 1.00 | 39.08 | C | |
| ATOM | 189 | O | PHE | A1027 | 15.470 | 49.929 | 10.175 | 1.00 | 38.67 | O | |
| ATOM | 190 | N | ALA | A1028 | 17.080 | 49.409 | 8.682 | 1.00 | 38.48 | N | |
| ATOM | 191 | CA | ALA | A1028 | 17.980 | 48.811 | 9.656 | 1.00 | 38.36 | C | |
| ATOM | 192 | CB | ALA | A1028 | 19.242 | 48.321 | 8.960 | 1.00 | 37.96 | C | |
| ATOM | 193 | C | ALA | A1028 | 18.349 | 49.784 | 10.764 | 1.00 | 38.25 | C | |
| ATOM | 194 | O | ALA | A1028 | 18.533 | 49.384 | 11.907 | 1.00 | 38.15 | O | |
| ATOM | 195 | N | LYS | A1029 | 18.456 | 51.061 | 10.418 | 1.00 | 38.38 | N | |
| ATOM | 196 | CA | LYS | A1029 | 18.823 | 52.094 | 11.380 | 1.00 | 38.44 | C | |
| ATOM | 197 | CB | LYS | A1029 | 19.154 | 53.391 | 10.636 | 1.00 | 39.34 | C | |
| ATOM | 198 | CG | LYS | A1029 | 19.997 | 54.393 | 11.411 | 1.00 | 40.55 | C | |
| ATOM | 199 | CD | LYS | A1029 | 19.248 | 55.005 | 12.591 | 1.00 | 41.77 | C | |
| ATOM | 200 | CE | LYS | A1029 | 20.043 | 56.146 | 13.215 | 1.00 | 41.81 | C | |
| ATOM | 201 | NZ | LYS | A1029 | 20.282 | 57.218 | 12.208 | 1.00 | 42.20 | N | |
| ATOM | 202 | C | LYS | A1029 | 17.703 | 52.331 | 12.386 | 1.00 | 37.98 | C | |
| ATOM | 203 | O | LYS | A1029 | 17.948 | 52.731 | 13.522 | 1.00 | 38.26 | O | |
| ATOM | 204 | N | ARG | A1030 | 16.470 | 52.084 | 11.969 | 1.00 | 37.55 | N | |
| ATOM | 205 | CA | ARG | A1030 | 15.330 | 52.267 | 12.856 | 1.00 | 37.18 | C | |
| ATOM | 206 | CB | ARG | A1030 | 14.050 | 52.489 | 12.040 | 1.00 | 38.15 | C | |
| ATOM | 207 | CG | ARG | A1030 | 13.833 | 53.934 | 11.573 | 1.00 | 39.87 | C | |
| ATOM | 208 | CD | ARG | A1030 | 12.449 | 54.110 | 10.937 | 1.00 | 40.62 | C | |
| ATOM | 209 | NE | ARG | A1030 | 12.383 | 53.532 | 9.598 | 1.00 | 41.54 | N | |
| ATOM | 210 | CZ | ARG | A1030 | 12.825 | 54.142 | 8.500 | 1.00 | 42.11 | C | |
| ATOM | 211 | NH1 | ARG | A1030 | 13.359 | 55.355 | 8.577 | 1.00 | 41.94 | N | |
| ATOM | 212 | NH2 | ARG | A1030 | 12.747 | 53.533 | 7.323 | 1.00 | 42.66 | N | |
| ATOM | 213 | C | ARG | A1030 | 15.145 | 51.064 | 13.780 | 1.00 | 36.44 | C | |
| ATOM | 214 | O | ARG | A1030 | 14.650 | 51.202 | 14.900 | 1.00 | 36.62 | O | |
| ATOM | 215 | N | VAL | A1031 | 15.560 | 49.889 | 13.313 | 1.00 | 35.30 | N | |
| ATOM | 216 | CA | VAL | A1031 | 15.417 | 48.655 | 14.080 | 1.00 | 34.12 | C | |
| ATOM | 217 | CB | VAL | A1031 | 15.160 | 47.458 | 13.145 | 1.00 | 34.29 | C | |
| ATOM | 218 | CG1 | VAL | A1031 | 14.965 | 46.190 | 13.963 | 1.00 | 34.93 | C | |
| ATOM | 219 | CG2 | VAL | A1031 | 13.949 | 47.723 | 12.279 | 1.00 | 34.77 | C | |
| ATOM | 220 | C | VAL | A1031 | 16.615 | 48.300 | 14.960 | 1.00 | 33.18 | C | |
| ATOM | 221 | O | VAL | A1031 | 16.447 | 47.763 | 16.057 | 1.00 | 33.04 | O | |
| ATOM | 222 | N | LEU | A1032 | 17.818 | 48.595 | 14.482 | 1.00 | 31.96 | N | |
| ATOM | 223 | CA | LEU | A1032 | 19.026 | 48.264 | 15.226 | 1.00 | 31.28 | C | |
| ATOM | 224 | CB | LEU | A1032 | 20.081 | 47.692 | 14.274 | 1.00 | 30.83 | C | |
| ATOM | 225 | CG | LEU | A1032 | 19.665 | 46.475 | 13.438 | 1.00 | 30.70 | C | |
| ATOM | 226 | CD1 | LEU | A1032 | 20.852 | 46.007 | 12.608 | 1.00 | 30.16 | C | |
| ATOM | 227 | CD2 | LEU | A1032 | 19.172 | 45.351 | 14.349 | 1.00 | 30.17 | C | |
| ATOM | 228 | C | LEU | A1032 | 19.634 | 49.422 | 16.005 | 1.00 | 30.91 | C | |
| ATOM | 229 | O | LEU | A1032 | 19.534 | 50.578 | 15.602 | 1.00 | 31.10 | O | |
| ATOM | 230 | N | THR | A1033 | 20.262 | 49.095 | 17.129 | 1.00 | 30.41 | N | |
| ATOM | 231 | CA | THR | A1033 | 20.918 | 50.094 | 17.960 | 1.00 | 29.93 | C | |
| ATOM | 232 | CB | THR | A1033 | 21.113 | 49.605 | 19.410 | 1.00 | 29.26 | C | |
| ATOM | 233 | OG1 | THR | A1033 | 22.084 | 48.552 | 19.438 | 1.00 | 28.24 | O | |
| ATOM | 234 | CG2 | THR | A1033 | 19.800 | 49.097 | 19.980 | 1.00 | 29.31 | C | |
| ATOM | 235 | C | THR | A1033 | 22.291 | 50.334 | 17.339 | 1.00 | 30.14 | C | |
| ATOM | 236 | O | THR | A1033 | 22.678 | 49.633 | 16.405 | 1.00 | 29.45 | O | |
| ATOM | 237 | N | ALA | A1034 | 23.018 | 51.321 | 17.856 | 1.00 | 30.49 | N | |
| ATOM | 238 | CA | ALA | A1034 | 24.344 | 5L638 | 17.339 | 1.00 | 31.05 | C | |
| ATOM | 239 | CB | ALA | A1034 | 24.987 | 52.734 | 18.177 | 1.00 | 30.92 | C | |
| ATOM | 240 | C | ALA | A1034 | 25.211 | 50.388 | 17.356 | 1.00 | 31.28 | C | |
| ATOM | 241 | O | ALA | A1034 | 25.837 | 50.047 | 16.357 | 1.00 | 31.20 | O | |
| ATOM | 242 | N | LEU | A1035 | 25.241 | 49.706 | 18.495 | 1.00 | 31.90 | N | |
| ATOM | 243 | CA | LEU | A1035 | 26.030 | 48.487 | 18.619 | 1.00 | 32.66 | C | |
| ATOM | 244 | CB | LEU | A1035 | 25.878 | 47.907 | 20.031 | 1.00 | 32.74 | C | |
| ATOM | 245 | CG | LEU | A1035 | 26.464 | 48.733 | 21.195 | 1.00 | 32.90 | C | |
| ATOM | 246 | CD1 | LEU | A1035 | 26.003 | 48.158 | 22.529 | 1.00 | 32.38 | C | |
| ATOM | 247 | CD2 | LEU | A1035 | 27.985 | 48.737 | 21.121 | 1.00 | 32.56 | C | |
| ATOM | 248 | C | LEU | A1035 | 25.612 | 47.454 | 17.556 | 1.00 | 33.36 | C | |
| ATOM | 249 | O | LEU | A1035 | 26.442 | 46.992 | 16.769 | 1.00 | 33.53 | O | |
| ATOM | 250 | N | GLU | A1036 | 24.328 | 47.104 | 17.526 | 1.00 | 33.64 | N | |
| ATOM | 251 | CA | GLU | A1036 | 23.822 | 46.140 | 16.555 | 1.00 | 34.14 | C | |
| ATOM | 252 | CB | GLU | A1036 | 22.299 | 46.014 | 16.682 | 1.00 | 33.87 | C | |
| ATOM | 253 | CG | GLU | A1036 | 21.818 | 45.217 | 17.890 | 1.00 | 33.65 | C | |
| ATOM | 254 | CD | GLU | A1036 | 20.341 | 45.462 | 18.216 | 1.00 | 33.37 | C | |
| ATOM | 255 | OE1 | GLU | A1036 | 19.726 | 44.613 | 18.895 | 1.00 | 32.88 | O | |
| ATOM | 256 | OE2 | GLU | A1036 | 19.797 | 46.509 | 17.808 | 1.00 | 33.01 | O | |
| ATOM | 257 | C | GLU | A1036 | 24.188 | 46.573 | 15.137 | 1.00 | 34.85 | C | |
| ATOM | 258 | O | GLU | A1036 | 24.526 | 45.752 | 14.284 | 1.00 | 34.59 | O | |
| ATOM | 259 | N | MET | A1037 | 24.120 | 47.875 | 14.893 | 1.00 | 35.84 | N | |
| ATOM | 260 | CA | MET | A1037 | 24.436 | 48.433 | 13.585 | 1.00 | 37.02 | C | |
| ATOM | 261 | CB | MET | A1037 | 24.172 | 49.936 | 13.605 | 1.00 | 37.11 | C | |
| ATOM | 262 | CG | MET | A1037 | 23.656 | 50.510 | 12.294 | 1.00 | 38.16 | C | |
| ATOM | 263 | SD | MET | A1037 | 22.148 | 49.717 | 11.685 | 1.00 | 38.53 | S | |
| ATOM | 264 | CE | MET | A1037 | 22.507 | 49.661 | 9.949 | 1.00 | 38.71 | C | |
| ATOM | 265 | C | MET | A1037 | 25.899 | 48.141 | 13.215 | 1.00 | 37.76 | C | |
| ATOM | 266 | O | MET | A1037 | 26.227 | 47.928 | 12.044 | 1.00 | 37.71 | O | |
| ATOM | 267 | N | GLU | A1038 | 26.774 | 48.117 | 14.217 | 1.00 | 38.51 | N | |
| ATOM | 268 | CA | GLU | A1038 | 28.183 | 47.835 | 13.977 | 1.00 | 39.30 | C | |
| ATOM | 269 | CB | GLU | A1038 | 28.989 | 47.966 | 15.275 | 1.00 | 40.38 | C | |
| ATOM | 270 | CG | GLU | A1038 | 28.963 | 49.369 | 15.880 | 1.00 | 41.32 | C | |
| ATOM | 271 | CD | GLU | A1038 | 29.874 | 49.513 | 17.086 | 1.00 | 42.00 | C | |
| ATOM | 272 | OE1 | GLU | A1038 | 29.789 | 50.553 | 17.771 | 1.00 | 42.33 | O | |
| ATOM | 273 | OE2 | GLU | A1038 | 30.677 | 48.592 | 17.349 | 1.00 | 42.44 | O | |
| ATOM | 274 | C | GLU | A1038 | 28.349 | 46.436 | 13.395 | 1.00 | 39.50 | C | |
| ATOM | 275 | O | GLU | A1038 | 28.932 | 46.272 | 12.327 | 1.00 | 40.05 | O | |
| ATOM | 276 | N | ARG | A1039 | 27.837 | 45.423 | 14.087 | 1.00 | 39.36 | N | |
| ATOM | 277 | CA | ARG | A1039 | 27.945 | 44.063 | 13.577 | 1.00 | 39.25 | C | |
| ATOM | 278 | CB | ARG | A1039 | 27.347 | 43.053 | 14.563 | 1.00 | 39.62 | C | |
| ATOM | 279 | CG | ARG | A1039 | 27.395 | 41.632 | 14.025 | 1.00 | 40.25 | C | |
| ATOM | 280 | CD | ARG | A1039 | 27.177 | 40.585 | 15.089 | 1.00 | 40.85 | C | |
| ATOM | 281 | NE | ARG | A1039 | 27.251 | 39.243 | 14.516 | 1.00 | 41.68 | N | |
| ATOM | 282 | CZ | ARG | A1039 | 28.314 | 38.756 | 13.879 | 1.00 | 42.00 | C | |
| ATOM | 283 | NH1 | ARG | A1039 | 28.289 | 37.523 | 13.387 | 1.00 | 42.01 | N | |
| ATOM | 284 | NH2 | ARG | A1039 | 29.408 | 39.496 | 13.735 | 1.00 | 41.92 | N | |
| ATOM | 285 | C | ARG | A1039 | 27.241 | 43.938 | 12.228 | 1.00 | 39.03 | C | |
| ATOM | 286 | O | ARG | A1039 | 27.774 | 43.347 | 11.291 | 1.00 | 38.64 | O | |
| ATOM | 287 | N | PHE | A1040 | 26.040 | 44.502 | 12.136 | 1.00 | 39.10 | N | |
| ATOM | 288 | CA | PHE | A1040 | 25.255 | 44.464 | 10.901 | 1.00 | 39.18 | C | |
| ATOM | 289 | CB | PHE | A1040 | 24.030 | 45.369 | 11.036 | 1.00 | 38.05 | C | |
| ATOM | 290 | CG | PHE | A1040 | 23.268 | 45.560 | 9.754 | 1.00 | 37.43 | C | |
| ATOM | 291 | CD1 | PHE | A1040 | 22.443 | 44.557 | 9.258 | 1.00 | 37.16 | C | |
| ATOM | 292 | CD2 | PHE | A1040 | 23.365 | 46.752 | 9.045 | 1.00 | 37.16 | C | |
| ATOM | 293 | CE1 | PHE | A1040 | 21.722 | 44.742 | 8.074 | 1.00 | 36.76 | C | |
| ATOM | 294 | CE2 | PHE | A1040 | 22.649 | 46.946 | 7.863 | 1.00 | 36.76 | C | |
| ATOM | 295 | CZ | PHE | A1040 | 21.825 | 45.936 | 7.379 | 1.00 | 36.61 | C | |
| ATOM | 296 | C | PHE | A1040 | 26.063 | 44.910 | 9.682 | 1.00 | 39.84 | C | |
| ATOM | 297 | O | PHE | A1040 | 26.157 | 44.190 | 8.682 | 1.00 | 39.67 | O | |
| ATOM | 298 | N | THR | A1041 | 26.643 | 46.104 | 9.774 | 1.00 | 40.57 | N | |
| ATOM | 299 | CA | THR | A1041 | 27.415 | 46.668 | 8.676 | 1.00 | 41.24 | C | |
| ATOM | 300 | CB | THR | A1041 | 27.726 | 48.138 | 8.938 | 1.00 | 41.14 | C | |
| ATOM | 301 | OG1 | THR | A1041 | 28.364 | 48.272 | 10.212 | 1.00 | 41.93 | O | |
| ATOM | 302 | CG2 | THR | A1041 | 26.446 | 48.948 | 8.933 | 1.00 | 40.95 | C | |
| ATOM | 303 | C | THR | A1041 | 28.708 | 45.931 | 8.353 | 1.00 | 41.72 | C | |
| ATOM | 304 | O | THR | A1041 | 29.251 | 46.080 | 7.259 | 1.00 | 41.99 | O | |
| ATOM | 305 | N | SER | A1042 | 29.198 | 45.133 | 9.297 | 1.00 | 42.15 | N | |
| ATOM | 306 | CA | SER | A1042 | 30.419 | 44.367 | 9.078 | 1.00 | 42.65 | C | |
| ATOM | 307 | CB | SER | A1042 | 31.106 | 44.057 | 10.407 | 1.00 | 42.64 | C | |
| ATOM | 308 | OG | SER | A1042 | 30.642 | 42.828 | 10.934 | 1.00 | 43.03 | O | |
| ATOM | 309 | C | SER | A1042 | 30.066 | 43.050 | 8.381 | 1.00 | 43.22 | C | |
| ATOM | 310 | O | SER | A1042 | 30.937 | 42.218 | 8.118 | 1.00 | 43.14 | O | |
| ATOM | 311 | N | LEU | A1043 | 28.781 | 42.869 | 8.095 | 1.00 | 43.80 | N | |
| ATOM | 312 | CA | LEU | A1043 | 28.298 | 41.660 | 7.439 | 1.00 | 44.32 | C | |
| ATOM | 313 | CB | LEU | A1043 | 27.151 | 41.040 | 8.247 | 1.00 | 43.98 | C | |
| ATOM | 314 | CG | LEU | A1043 | 27.491 | 40.512 | 9.642 | 1.00 | 43.88 | C | |
| ATOM | 315 | CD1 | LEU | A1043 | 26.219 | 40.165 | 10.388 | 1.00 | 43.76 | C | |
| ATOM | 316 | CD2 | LEU | A1043 | 28.383 | 39.294 | 9.521 | 1.00 | 44.03 | C | |
| ATOM | 317 | C | LEU | A1043 | 27.821 | 41.973 | 6.026 | 1.00 | 44.90 | C | |
| ATOM | 318 | O | LEU | A1043 | 27.448 | 43.110 | 5.722 | 1.00 | 44.88 | O | |
| ATOM | 319 | N | LYS | A1044 | 27.827 | 40.950 | 5.174 | 1.00 | 45.38 | N | |
| ATOM | 320 | CA | LYS | A1044 | 27.412 | 41.088 | 3.782 | 1.00 | 45.95 | C | |
| ATOM | 321 | CB | LYS | A1044 | 28.556 | 40.673 | 2.856 | 1.00 | 46.91 | C | |
| ATOM | 322 | CG | LYS | A1044 | 28.976 | 39.209 | 3.060 | 1.00 | 47.97 | C | |
| ATOM | 323 | CD | LYS | A1044 | 29.803 | 38.669 | 1.896 | 1.00 | 48.98 | C | |
| ATOM | 324 | CE | LYS | A1044 | 30.319 | 37.260 | 2.187 | 1.00 | 49.35 | C | |
| ATOM | 325 | NZ | LYS | A1044 | 29.216 | 36.313 | 2.505 | 1.00 | 49.24 | N | |
| ATOM | 326 | C | LYS | A1044 | 26.202 | 40.217 | 3.457 | 1.00 | 45.75 | C | |
| ATOM | 327 | O | LYS | A1044 | 25.779 | 39.391 | 4.266 | 1.00 | 46.06 | O | |
| ATOM | 328 | N | GLY | A1045 | 25.674 | 40.416 | 2.252 | 1.00 | 45.39 | N | |
| ATOM | 329 | CA | GLY | A1045 | 24.538 | 39.663 | 1.743 | 1.00 | 44.96 | C | |
| ATOM | 330 | C | GLY | A1045 | 23.577 | 38.960 | 2.685 | 1.00 | 44.52 | C | |
| ATOM | 331 | O | GLY | A1045 | 23.147 | 39.520 | 3.692 | 1.00 | 44.43 | O | |
| ATOM | 332 | N | ARG | A1046 | 23.231 | 37.726 | 2.333 | 1.00 | 44.21 | N | |
| ATOM | 333 | CA | ARG | A1046 | 22.297 | 36.917 | 3.112 | 1.00 | 43.89 | C | |
| ATOM | 334 | CB | ARG | A1046 | 22.304 | 35.474 | 2.589 | 1.00 | 44.58 | C | |
| ATOM | 335 | CG | ARG | A1046 | 21.962 | 35.399 | 1.099 | 1.00 | 46.46 | C | |
| ATOM | 336 | CD | ARG | A1046 | 21.891 | 33.977 | 0.547 | 1.00 | 47.97 | C | |
| ATOM | 337 | NE | ARG | A1046 | 20.698 | 33.253 | 0.979 | 1.00 | 49.25 | N | |
| ATOM | 338 | CZ | ARG | A1046 | 20.360 | 32.043 | 0.538 | 1.00 | 49.83 | C | |
| ATOM | 339 | NH1 | ARG | A1046 | 19.257 | 31.454 | 0.983 | 1.00 | 49.98 | N | |
| ATOM | 340 | NH2 | ARG | A1046 | 21.123 | 31.420 | −0.352 | 1.00 | 49.87 | N | |
| ATOM | 341 | C | ARG | A1046 | 22.572 | 36.951 | 4.609 | 1.00 | 43.05 | C | |
| ATOM | 342 | O | ARG | A1046 | 21.657 | 37.167 | 5.402 | 1.00 | 42.92 | O | |
| ATOM | 343 | N | ARG | A1047 | 23.830 | 36.751 | 4.994 | 1.00 | 42.04 | N | |
| ATOM | 344 | CA | ARG | A1047 | 24.212 | 36.775 | 6.401 | 1.00 | 40.92 | C | |
| ATOM | 345 | CB | ARG | A1047 | 25.726 | 36.592 | 6.553 | 1.00 | 41.86 | C | |
| ATOM | 346 | CG | ARG | A1047 | 26.196 | 35.146 | 6.581 | 1.00 | 43.21 | C | |
| ATOM | 347 | CD | ARG | A1047 | 27.613 | 35.070 | 7.134 | 1.00 | 44.61 | C | |
| ATOM | 348 | NE | ARG | A1047 | 28.023 | 33.709 | 7.475 | 1.00 | 45.70 | N | |
| ATOM | 349 | CZ | ARG | A1047 | 28.346 | 32.773 | 6.588 | 1.00 | 46.26 | C | |
| ATOM | 350 | NH1 | ARG | A1047 | 28.705 | 31.565 | 7.004 | 1.00 | 46.85 | N | |
| ATOM | 351 | NH2 | ARG | A1047 | 28.318 | 33.042 | 5.289 | 1.00 | 46.67 | N | |
| ATOM | 352 | C | ARG | A1047 | 23.800 | 38.082 | 7.074 | 1.00 | 39.44 | C | |
| ATOM | 353 | O | ARG | A1047 | 23.397 | 38.100 | 8.236 | 1.00 | 38.89 | O | |
| ATOM | 354 | N | GLN | A1048 | 23.914 | 39.178 | 6.338 | 1.00 | 37.81 | N | |
| ATOM | 355 | CA | GLN | A1048 | 23.550 | 40.475 | 6.871 | 1.00 | 36.41 | C | |
| ATOM | 356 | CB | GLN | A1048 | 24.011 | 41.576 | 5.920 | 1.00 | 36.74 | C | |
| ATOM | 357 | CG | GLN | A1048 | 24.106 | 42.940 | 6.565 | 1.00 | 37.57 | C | |
| ATOM | 358 | CD | GLN | A1048 | 24.476 | 44.024 | 5.578 | 1.00 | 38.09 | C | |
| ATOM | 359 | OE1 | GLN | A1048 | 24.860 | 45.132 | 5.965 | 1.00 | 38.67 | O | |
| ATOM | 360 | NE2 | CLN | A1048 | 24.351 | 43.717 | 4.295 | 1.00 | 38.03 | N | |
| ATOM | 361 | C | GLN | A1048 | 22.037 | 40.561 | 7.074 | 1.00 | 35.21 | C | |
| ATOM | 362 | O | GLN | A1048 | 21.567 | 41.201 | 8.015 | 1.00 | 34.68 | O | |
| ATOM | 363 | N | ILE | A1049 | 21.279 | 39.919 | 6.189 | 1.00 | 33.75 | N | |
| ATOM | 364 | CA | ILE | A1049 | 19.826 | 39.934 | 6.295 | 1.00 | 32.76 | C | |
| ATOM | 365 | CB | ILE | A1049 | 19.146 | 39.436 | 4.987 | 1.00 | 32.88 | C | |
| ATOM | 366 | CG2 | ILE | A1049 | 17.628 | 39.437 | 5.153 | 1.00 | 32.63 | C | |
| ATOM | 367 | CG1 | ILE | A1049 | 19.536 | 40.333 | 3.808 | 1.00 | 32.63 | C | |
| ATOM | 368 | CD1 | ILE | A1049 | 19.070 | 41.776 | 3.930 | 1.00 | 32.59 | C | |
| ATOM | 369 | C | ILE | A1049 | 19.366 | 39.062 | 7.466 | 1.00 | 31.99 | C | |
| ATOM | 370 | O | ILE | A1049 | 18.480 | 39.456 | 8.223 | 1.00 | 31.76 | O | |
| ATOM | 371 | N | GLU | A1050 | 19.964 | 37.883 | 7.611 | 1.00 | 31.37 | N | |
| ATOM | 372 | CA | GLU | A1050 | 19.610 | 36.982 | 8.707 | 1.00 | 31.34 | C | |
| ATOM | 373 | CB | GLU | A1050 | 20.362 | 35.648 | 8.591 | 1.00 | 32.61 | C | |
| ATOM | 374 | CG | GLU | A1050 | 20.706 | 34.980 | 9.944 | 1.00 | 36.11 | C | |
| ATOM | 375 | CD | GLU | A1050 | 19.482 | 34.642 | 10.819 | 1.00 | 37.78 | C | |
| ATOM | 376 | OE1 | GLU | A1050 | 19.674 | 34.340 | 12.024 | 1.00 | 39.05 | O | |
| ATOM | 377 | OE2 | GLU | A1050 | 18.339 | 34.671 | 10.311 | 1.00 | 38.44 | O | |
| ATOM | 378 | C | GLU | A1050 | 19.929 | 37.642 | 10.047 | 1.00 | 30.27 | C | |
| ATOM | 379 | O | GLU | A1050 | 19.172 | 37.494 | 11.014 | 1.00 | 30.09 | O | |
| ATOM | 380 | N | TYR | A1051 | 21.041 | 38.372 | 10.107 | 1.00 | 28.49 | N | |
| ATOM | 381 | CA | TYR | A1051 | 21.398 | 39.040 | 11.343 | 1.00 | 27.23 | C | |
| ATOM | 382 | CB | TYR | A1051 | 22.747 | 39.750 | 11.244 | 1.00 | 26.82 | C | |
| ATOM | 383 | CG | TYR | A1051 | 23.032 | 40.565 | 12.484 | 1.00 | 26.72 | C | |
| ATOM | 384 | CD1 | TYR | A1051 | 22.657 | 41.906 | 12.569 | 1.00 | 26.89 | C | |
| ATOM | 385 | CE1 | TYR | A1051 | 22.830 | 42.627 | 13.750 | 1.00 | 26.37 | C | |
| ATOM | 386 | CD2 | TYR | A1051 | 23.590 | 39.970 | 13.612 | 1.00 | 26.66 | C | |
| ATOM | 387 | CE2 | TYR | A1051 | 23.761 | 40.676 | 14.786 | 1.00 | 26.28 | C | |
| ATOM | 388 | CZ | TYR | A1051 | 23.381 | 42.000 | 14.852 | 1.00 | 26.29 | C | |
| ATOM | 389 | OH | TYR | A1051 | 23.554 | 42.688 | 16.031 | 1.00 | 26.92 | O | |
| ATOM | 390 | C | TYR | A1051 | 20.332 | 40.069 | 11.670 | 1.00 | 26.31 | C | |
| ATOM | 391 | O | TYR | A1051 | 19.893 | 40.189 | 12.811 | 1.00 | 25.81 | O | |
| ATOM | 392 | N | LEU | A1052 | 19.927 | 40.812 | 10.650 | 1.00 | 25.22 | N | |
| ATOM | 393 | CA | LEU | A1052 | 18.923 | 41.845 | 10.809 | 1.00 | 24.40 | C | |
| ATOM | 394 | CB | LEU | A1052 | 18.854 | 42.685 | 9.532 | 1.00 | 24.44 | C | |
| ATOM | 395 | CG | LEU | A1052 | 17.719 | 43.703 | 9.465 | 1.00 | 25.24 | C | |
| ATOM | 396 | CD1 | LEU | A1052 | 17.904 | 44.780 | 10.533 | 1.00 | 24.85 | C | |
| ATOM | 397 | CD2 | LEU | A1052 | 17.692 | 44.314 | 8.081 | 1.00 | 25.55 | C | |
| ATOM | 398 | C | LEU | A1052 | 17.551 | 41.242 | 11.126 | 1.00 | 23.78 | C | |
| ATOM | 399 | O | LEU | A1052 | 16.808 | 41.771 | 11.957 | 1.00 | 23.28 | O | |
| ATOM | 400 | N | ALA | A1053 | 17.227 | 40.134 | 10.459 | 1.00 | 22.87 | N | |
| ATOM | 401 | CA | ALA | A1053 | 15.948 | 39.459 | 10.655 | 1.00 | 22.13 | C | |
| ATOM | 402 | CB | ALA | A1053 | 15.780 | 38.354 | 9.619 | 1.00 | 21.79 | C | |
| ATOM | 403 | C | ALA | A1053 | 15.858 | 38.875 | 12.066 | 1.00 | 21.81 | C | |
| ATOM | 404 | O | ALA | A1053 | 14.841 | 39.022 | 12.748 | 1.00 | 21.24 | O | |
| ATOM | 405 | N | GLY | A1054 | 16.932 | 38.215 | 12.497 | 1.00 | 21.35 | N | |
| ATOM | 406 | CA | GLY | A1054 | 16.953 | 37.624 | 13.819 | 1.00 | 20.95 | C | |
| ATOM | 407 | C | GLY | A1054 | 16.707 | 38.649 | 14.910 | 1.00 | 20.80 | C | |
| ATOM | 408 | O | GLY | A1054 | 15.890 | 38.428 | 15.803 | 1.00 | 20.64 | O | |
| ATOM | 409 | N | ARG | A1055 | 17.419 | 39.769 | 14.845 | 1.00 | 20.73 | N | |
| ATOM | 410 | CA | ARG | A1055 | 17.255 | 40.822 | 15.843 | 1.00 | 21.15 | C | |
| ATOM | 411 | CB | ARG | A1055 | 18.254 | 41.959 | 15.586 | 1.00 | 20.76 | C | |
| ATOM | 412 | CG | ARG | A1055 | 19.514 | 41.893 | 16.444 | 1.00 | 20.45 | C | |
| ATOM | 413 | CD | ARG | A1055 | 20.238 | 40.562 | 16.281 | 1.00 | 20.93 | C | |
| ATOM | 414 | NE | ARG | A1055 | 21.323 | 40.413 | 17.249 | 1.00 | 20.92 | N | |
| ATOM | 415 | CZ | ARG | A1055 | 22.109 | 39.344 | 17.337 | 1.00 | 21.08 | C | |
| ATOM | 416 | NH1 | ARG | A1055 | 21.937 | 38.314 | 16.515 | 1.00 | 21.54 | N | |
| ATOM | 417 | NH2 | ARG | A1055 | 23.073 | 39.308 | 18.246 | 1.00 | 20.48 | N | |
| ATOM | 418 | C | ARG | A1055 | 15.822 | 41.355 | 15.834 | 1.00 | 20.82 | C | |
| ATOM | 419 | O | ARG | A1055 | 15.235 | 41.597 | 16.884 | 1.00 | 19.96 | O | |
| ATOM | 420 | N | TRP | A1056 | 15.272 | 41.535 | 14.638 | 1.00 | 21.33 | N | |
| ATOM | 421 | CA | TRP | A1056 | 13.907 | 42.018 | 14.481 | 1.00 | 21.83 | C | |
| ATOM | 422 | CB | TRP | A1056 | 13.597 | 42.252 | 12.995 | 1.00 | 23.26 | C | |
| ATOM | 423 | CG | TRP | A1056 | 12.117 | 42.342 | 12.688 | 1.00 | 25.60 | C | |
| ATOM | 424 | CD2 | TRP | A1056 | 11.384 | 43.518 | 12.326 | 1.00 | 26.69 | C | |
| ATOM | 425 | CE2 | TRP | A1056 | 10.026 | 43.140 | 12.186 | 1.00 | 27.05 | C | |
| ATOM | 426 | CE3 | TRP | A1056 | 11.741 | 44.852 | 12.106 | 1.00 | 27.71 | C | |
| ATOM | 427 | CD1 | TRP | A1056 | 11.199 | 41.324 | 12.745 | 1.00 | 26.25 | C | |
| ATOM | 428 | NE1 | TRP | A1056 | 9.942 | 41.798 | 12.448 | 1.00 | 27.38 | N | |
| ATOM | 429 | CZ2 | TRP | A1056 | 9.025 | 44.048 | 11.839 | 1.00 | 28.05 | C | |
| ATOM | 430 | CZ3 | TRP | A1056 | 10.741 | 45.763 | 11.757 | 1.00 | 29.41 | C | |
| ATOM | 431 | CH2 | TRP | A1056 | 9.397 | 45.353 | 11.628 | 1.00 | 28.81 | C | |
| ATOM | 432 | C | TRP | A1056 | 12.956 | 40.973 | 15.059 | 1.00 | 21.40 | C | |
| ATOM | 433 | O | TRP | A1056 | 11.999 | 41.299 | 15.754 | 1.00 | 21.32 | O | |
| ATOM | 434 | N | SER | A1057 | 13.233 | 39.714 | 14.752 | 1.00 | 20.70 | N | |
| ATOM | 435 | CA | SER | A1057 | 12.429 | 38.605 | 15.231 | 1.00 | 20.76 | C | |
| ATOM | 436 | CB | SER | A1057 | 12.956 | 37.293 | 14.632 | 1.00 | 21.29 | C | |
| ATOM | 437 | OG | SER | A1057 | 12.032 | 36.234 | 14.815 | 1.00 | 23.54 | O | |
| ATOM | 438 | C | SER | A1057 | 12.498 | 38.559 | 16.760 | 1.00 | 20.11 | C | |
| ATOM | 439 | O | SER | A1057 | 11.484 | 38.361 | 17.438 | 1.00 | 19.93 | O | |
| ATOM | 440 | N | ALA | A1058 | 13,693 | 38.750 | 17.303 | 1.00 | 19.35 | N | |
| ATOM | 441 | CA | ALA | A1058 | 13.881 | 38.727 | 18.751 | 1.00 | 18.89 | C | |
| ATOM | 442 | CB | ALA | A1058 | 15.373 | 38.780 | 19.098 | 1.00 | 18.02 | C | |
| ATOM | 443 | C | ALA | A1058 | 13.139 | 39.865 | 19.448 | 1.00 | 18.51 | C | |
| ATOM | 444 | O | ALA | A1058 | 12.502 | 39.657 | 20.482 | 1.00 | 17.31 | O | |
| ATOM | 445 | N | LYS | A1059 | 13.213 | 41.062 | 18.875 | 1.00 | 19.01 | N | |
| ATOM | 446 | CA | LYS | A1059 | 12.560 | 42.230 | 19.470 | 1.00 | 20.07 | C | |
| ATOM | 447 | CB | LYS | A1059 | 13.026 | 43.513 | 18.771 | 1.00 | 19.92 | C | |
| ATOM | 448 | CG | LYS | A1059 | 14.539 | 43.671 | 18.811 | 1.00 | 20.81 | C | |
| ATOM | 449 | CD | LYS | A1059 | 15.003 | 45.101 | 18.534 | 1.00 | 21.35 | C | |
| ATOM | 450 | CE | LYS | A1059 | 16.530 | 45.190 | 18.538 | 1.00 | 21.30 | C | |
| ATOM | 451 | NZ | LYS | A1059 | 17.028 | 46.597 | 18.453 | 1.00 | 21.29 | N | |
| ATOM | 452 | C | LYS | A1059 | 11.045 | 42.112 | 19.420 | 1.00 | 20.60 | C | |
| ATOM | 453 | O | LYS | A1059 | 10.341 | 42.601 | 20.309 | 1.00 | 19.75 | O | |
| ATOM | 454 | N | GLU | A1060 | 10.550 | 41.448 | 18.381 | 1.00 | 21.44 | N | |
| ATOM | 455 | CA | GLU | A1060 | 9.118 | 41.246 | 18.234 | 1.00 | 22.69 | C | |
| ATOM | 456 | CB | GLU | A1060 | 8.783 | 40.742 | 16.832 | 1.00 | 24.22 | C | |
| ATOM | 457 | CG | GLU | A1060 | 7.303 | 40.761 | 16.521 | 1.00 | 27.42 | C | |
| ATOM | 458 | CD | GLU | A1060 | 6.730 | 42.169 | 16.542 | 1.00 | 29.59 | C | |
| ATOM | 459 | OE1 | GLU | A1060 | 7.314 | 43.053 | 15.866 | 1.00 | 29.73 | O | |
| ATOM | 460 | OE2 | GLU | A1060 | 5.700 | 42.386 | 17.229 | 1.00 | 30.63 | O | |
| ATOM | 461 | C | GLU | A1060 | 8.651 | 40.220 | 19.263 | 1.00 | 22.63 | C | |
| ATOM | 462 | O | GLU | A1060 | 7.631 | 40.418 | 19.932 | 1.00 | 22.87 | O | |
| ATOM | 463 | N | ALA | A1061 | 9.395 | 39.125 | 19.391 | 1.00 | 22.05 | N | |
| ATOM | 464 | CA | ALA | A1061 | 9.012 | 38.091 | 20.346 | 1.00 | 22.06 | C | |
| ATOM | 465 | CB | ALA | A1061 | 9.967 | 36.899 | 20.266 | 1.00 | 21.94 | C | |
| ATOM | 466 | C | ALA | A1061 | 8.997 | 38.663 | 21.755 | 1.00 | 22.04 | C | |
| ATOM | 467 | O | ALA | A1061 | 8.106 | 38.359 | 22.539 | 1.00 | 21.13 | O | |
| ATOM | 468 | N | PHE | A1062 | 9.978 | 39.506 | 22.063 | 1.00 | 22.38 | N | |
| ATOM | 469 | CA | PHE | A1062 | 10.055 | 40.119 | 23.380 | 1.00 | 23.29 | C | |
| ATOM | 470 | CB | PHE | A1062 | 11.348 | 40.918 | 23.532 | 1.00 | 22.75 | C | |
| ATOM | 471 | CG | PHE | A1062 | 11.440 | 41.648 | 24.833 | 1.00 | 22.71 | C | |
| ATOM | 472 | CD1 | PHE | A1062 | 10.804 | 42.876 | 25.003 | 1.00 | 22.28 | C | |
| ATOM | 473 | CD2 | PHE | A1062 | 12.107 | 41.080 | 25.914 | 1.00 | 22.60 | C | |
| ATOM | 474 | CE1 | PHE | A1062 | 10.828 | 43.529 | 26.232 | 1.00 | 22.88 | C | |
| ATOM | 475 | CE2 | PHE | A1062 | 12.138 | 41.722 | 27.154 | 1.00 | 22.54 | C | |
| ATOM | 476 | CZ | PHE | A1062 | 11.497 | 42.951 | 27.314 | 1.00 | 22.78 | C | |
| ATOM | 477 | C | PHE | A1062 | 8.865 | 41.030 | 23.667 | 1.00 | 24.27 | C | |
| ATOM | 478 | O | PHE | A1062 | 8.302 | 41.002 | 24.765 | 1.00 | 24.37 | O | |
| ATOM | 479 | N | SER | A1063 | 8.498 | 41.843 | 22.681 | 1.00 | 25.39 | N | |
| ATOM | 480 | CA | SER | A1063 | 7.373 | 42.751 | 22.818 | 1.00 | 26.79 | C | |
| ATOM | 481 | CB | SER | A1063 | 7.196 | 43.580 | 21.542 | 1.00 | 27.55 | C | |
| ATOM | 482 | OG | SER | A1063 | 8.184 | 44.598 | 21.464 | 1.00 | 28.80 | O | |
| ATOM | 483 | C | SER | A1063 | 6.108 | 41.965 | 23.094 | 1.00 | 27.26 | C | |
| ATOM | 484 | O | SER | A1063 | 5.271 | 42.366 | 23.897 | 1.00 | 27.47 | O | |
| ATOM | 485 | N | LYS | A1064 | 5.963 | 40.840 | 22.421 | 1.00 | 28.03 | N | |
| ATOM | 486 | CA | LYS | A1064 | 4.793 | 40.018 | 22.637 | 1.00 | 29.08 | C | |
| ATOM | 487 | CB | LYS | A1064 | 4.725 | 38.936 | 21.566 | 1.00 | 28.90 | C | |
| ATOM | 488 | CG | LYS | A1064 | 4.426 | 39.521 | 20.195 | 1.00 | 30.31 | C | |
| ATOM | 489 | CD | LYS | A1064 | 4.605 | 38.519 | 19.075 | 1.00 | 31.26 | C | |
| ATOM | 490 | CE | LYS | A1064 | 3.733 | 37.309 | 19.269 | 1.00 | 32.24 | C | |
| ATOM | 491 | NZ | LYS | A1064 | 3.887 | 36.361 | 18.141 | 1.00 | 33.56 | N | |
| ATOM | 492 | C | LYS | A1064 | 4.866 | 39.428 | 24.043 | 1.00 | 29.71 | C | |
| ATOM | 493 | O | LYS | A1064 | 3.886 | 39.467 | 24.788 | 1.00 | 29.61 | O | |
| ATOM | 494 | N | ALA | A1065 | 6.043 | 38.926 | 24.413 | 1.00 | 30.48 | N | |
| ATOM | 495 | CA | ALA | A1065 | 6.263 | 38.341 | 25.732 | 1.00 | 31.56 | C | |
| ATOM | 496 | CB | ALA | A1065 | 7.739 | 38.022 | 25.917 | 1.00 | 31.00 | C | |
| ATOM | 497 | C | ALA | A1065 | 5.780 | 39.269 | 26.851 | 1.00 | 32.65 | C | |
| ATOM | 498 | O | ALA | A1065 | 5.300 | 38.807 | 27.882 | 1.00 | 31.64 | O | |
| ATOM | 499 | N | MET | A1066 | 5.922 | 40.574 | 26.646 | 1.00 | 34.63 | N | |
| ATOM | 500 | CA | MET | A1066 | 5.479 | 41.557 | 27.630 | 1.00 | 37.05 | C | |
| ATOM | 501 | CB | MET | A1066 | 6.245 | 42.874 | 27.458 | 1.00 | 37.61 | C | |
| ATOM | 502 | CG | MET | A1066 | 7.710 | 42.808 | 27.867 | 1.00 | 39.52 | C | |
| ATOM | 503 | SD | MET | A1066 | 8.007 | 43.039 | 29.649 | 1.00 | 41.34 | S | |
| ATOM | 504 | CE | MET | A1066 | 8.983 | 44.550 | 29.647 | 1.00 | 40.03 | C | |
| ATOM | 505 | C | MET | A1066 | 3.985 | 41.798 | 27.435 | 1.00 | 38.24 | C | |
| ATOM | 506 | O | MET | A1066 | 3.313 | 42.364 | 28.295 | 1.00 | 38.31 | O | |
| ATOM | 507 | N | GLY | A1067 | 3.473 | 41.353 | 26.293 | 1.00 | 39.97 | N | |
| ATOM | 508 | CA | GLY | A1067 | 2.065 | 41.524 | 25.996 | 1.00 | 42.23 | C | |
| ATOM | 509 | C | GLY | A1067 | 1.716 | 42.953 | 25.623 | 1.00 | 43.61 | C | |
| ATOM | 510 | O | GLY | A1067 | 1.055 | 43.658 | 26.383 | 1.00 | 44.09 | O | |
| ATOM | 511 | N | THR | A1068 | 2.167 | 43.387 | 24,452 | 1.00 | 44.84 | N | |
| ATOM | 512 | CA | THR | A1068 | 1.889 | 44.739 | 23.985 | 1.00 | 46.14 | C | |
| ATOM | 513 | CB | THR | A1068 | 3.165 | 45.419 | 23.444 | 1.00 | 45.80 | C | |
| ATOM | 514 | OG1 | THR | A1068 | 3.639 | 44.703 | 22.297 | 1.00 | 45.63 | O | |
| ATOM | 515 | CG2 | THR | A1068 | 4.249 | 45.434 | 24.500 | 1.00 | 45.44 | C | |
| ATOM | 516 | C | THR | A1068 | 0.854 | 44.654 | 22.868 | 1.00 | 47.39 | C | |
| ATOM | 517 | O | THR | A1068 | 0.473 | 45.661 | 22.270 | 1.00 | 47.66 | O | |
| ATOM | 518 | N | GLY | A1069 | 0.409 | 43.432 | 22.592 | 1.00 | 48.83 | N | |
| ATOM | 519 | CA | GLY | A1069 | 0.585 | 43.211 | 21.555 | 1.00 | 50.40 | C | |
| ATOM | 520 | C | GLY | A1069 | 0.073 | 43.511 | 20.162 | 1.00 | 51.38 | C | |
| ATOM | 521 | O | GLY | A1069 | 0.661 | 42.716 | 19.569 | 1.00 | 51.45 | O | |
| ATOM | 522 | N | ILE | A1070 | 0.473 | 44.665 | 19.637 | 1.00 | 52.38 | N | |
| ATOM | 523 | CA | ILE | A1070 | 0.061 | 45.102 | 18.308 | 1.00 | 53.00 | C | |
| ATOM | 524 | CB | ILE | A1070 | 1.285 | 45.483 | 17.444 | 1.00 | 53.24 | C | |
| ATOM | 525 | CG2 | ILE | A1070 | 2.148 | 44.245 | 17.194 | 1.00 | 53.03 | C | |
| ATOM | 526 | CG1 | ILE | A1070 | 2.102 | 46.575 | 18.145 | 1.00 | 53.21 | C | |
| ATOM | 527 | CD1 | ILE | A1070 | 3.310 | 47.043 | 17.350 | 1.00 | 53.51 | C | |
| ATOM | 528 | C | ILE | A1070 | 0.850 | 46.316 | 18.471 | 1.00 | 53.30 | C | |
| ATOM | 529 | O | ILE | A1070 | 1.800 | 46.506 | 17.705 | 1.00 | 53.48 | O | |
| ATOM | 530 | N | SER | A1071 | 0.544 | 47.137 | 19.473 | 1.00 | 53.35 | N | |
| ATOM | 531 | CA | SER | A1071 | 1.344 | 48.321 | 19.771 | 1.00 | 53.45 | C | |
| ATOM | 532 | CB | SER | A1071 | 0.643 | 49.183 | 20.816 | 1.00 | 53.58 | C | |
| ATOM | 533 | OG | SER | A1071 | 0.577 | 48.494 | 22.058 | 1.00 | 54.00 | O | |
| ATOM | 534 | C | SER | A1071 | 2.631 | 47.765 | 20.360 | 1.00 | 53.38 | C | |
| ATOM | 535 | O | SER | A1071 | 2.596 | 46.720 | 21.019 | 1.00 | 53.61 | O | |
| ATOM | 536 | N | LYS | A1072 | 3.763 | 48.431 | 20.138 | 1.00 | 52.81 | N | |
| ATOM | 537 | CA | LYS | A1072 | 5.019 | 47.908 | 20.680 | 1.00 | 52.05 | C | |
| ATOM | 538 | CB | LYS | A1072 | 5.857 | 47.243 | 19.575 | 1.00 | 52.53 | C | |
| ATOM | 539 | CG | LYS | A1072 | 5.068 | 46.578 | 18.459 | 1.00 | 53.49 | C | |
| ATOM | 540 | CD | LYS | A1072 | 4.079 | 45.535 | 18.964 | 1.00 | 54.32 | C | |
| ATOM | 541 | CE | LYS | A1072 | 4.750 | 44.344 | 19.619 | 1.00 | 54.90 | C | |
| ATOM | 542 | NZ | LYS | A1072 | 3.722 | 43.377 | 20.117 | 1.00 | 55.42 | N | |
| ATOM | 543 | C | LYS | A1072 | 5.897 | 48.936 | 21.383 | 1.00 | 51.05 | C | |
| ATOM | 544 | O | LYS | A1072 | 5.587 | 50.127 | 21.442 | 1.00 | 50.60 | O | |
| ATOM | 545 | N | LEU | A1073 | 7.002 | 48.435 | 21.923 | 1.00 | 50.01 | N | |
| ATOM | 546 | CA | LEU | A1073 | 7.989 | 49.254 | 22.599 | 1.00 | 48.81 | C | |
| ATOM | 547 | CB | LEU | A1073 | 8.785 | 48.409 | 23.601 | 1.00 | 49.31 | C | |
| ATOM | 548 | CG | LEU | A1073 | 8.166 | 48.084 | 24.969 | 1.00 | 49.63 | C | |
| ATOM | 549 | CD1 | LEU | A1073 | 6.806 | 47,433 | 24.812 | 1.00 | 49.83 | C | |
| ATOM | 550 | CD2 | LEU | A1073 | 9.099 | 47.160 | 25.720 | 1.00 | 50.04 | C | |
| ATOM | 551 | C | LEU | A1073 | 8.930 | 49.779 | 21.521 | 1.00 | 47.79 | C | |
| ATOM | 552 | O | LEU | A1073 | 8.923 | 49.294 | 20.383 | 1.00 | 47.71 | O | |
| ATOM | 553 | N | GLY | A1074 | 9.733 | 50.776 | 21.869 | 1.00 | 46.44 | N | |
| ATOM | 554 | CA | GLY | A1074 | 10.670 | 51.301 | 20.896 | 1.00 | 44.58 | C | |
| ATOM | 555 | C | GLY | A1074 | 11.722 | 50.244 | 20.621 | 1.00 | 43.12 | C | |
| ATOM | 556 | O | GLY | A1074 | 12.315 | 49.697 | 21.553 | 1.00 | 43.10 | O | |
| ATOM | 557 | N | PHE | A1075 | 11.949 | 49.934 | 19.351 | 1.00 | 41.71 | N | |
| ATOM | 558 | CA | PHE | A1075 | 12.949 | 48.937 | 19.006 | 1.00 | 40.26 | C | |
| ATOM | 559 | CB | PHE | A1075 | 12.870 | 48.613 | 17.514 | 1.00 | 41.07 | C | |
| ATOM | 560 | CG | PHE | A1075 | 11.958 | 47.459 | 17.207 | 1.00 | 42.00 | C | |
| ATOM | 561 | CD1 | PHE | A1075 | 10.930 | 47.112 | 18.096 | 1.00 | 42.62 | C | |
| ATOM | 562 | CD2 | PHE | A1075 | 12.124 | 46.707 | 16.048 | 1.00 | 42.28 | C | |
| ATOM | 563 | CE1 | PHE | A1075 | 10.081 | 46.026 | 17.835 | 1.00 | 42.81 | C | |
| ATOM | 564 | CE2 | PHE | A1075 | 11.281 | 45.617 | 15.771 | 1.00 | 42.42 | C | |
| ATOM | 565 | CZ | PHE | A1075 | 10.261 | 45.276 | 16.666 | 1.00 | 42.86 | C | |
| ATOM | 566 | C | PHE | A1075 | 14.346 | 49.388 | 19.413 | 1.00 | 38.94 | C | |
| ATOM | 567 | O | PHE | A1075 | 15.257 | 48.569 | 19.552 | 1.00 | 38.45 | O | |
| ATOM | 568 | N | GLN | A1076 | 14.503 | 50.692 | 19.623 | 1.00 | 37.14 | N | |
| ATOM | 569 | CA | GLN | A1076 | 15.779 | 51.244 | 20.051 | 1.00 | 35.77 | C | |
| ATOM | 570 | CB | GLN | A1076 | 15.864 | 52.739 | 19.720 | 1.00 | 35.98 | C | |
| ATOM | 571 | CG | GLN | A1076 | 16.064 | 53.046 | 18.240 | 1.00 | 35.74 | C | |
| ATOM | 572 | CD | GLN | A1076 | 17.323 | 52.418 | 17.683 | 1.00 | 35.92 | C | |
| ATOM | 573 | OE1 | GLN | A1076 | 18.429 | 52.672 | 18.169 | 1.00 | 36.68 | O | |
| ATOM | 574 | NE2 | GLN | A1076 | 17.166 | 51.592 | 16.659 | 1.00 | 35.79 | N | |
| ATOM | 575 | C | GLN | A1076 | 15.927 | 51.043 | 21.556 | 1.00 | 34.84 | C | |
| ATOM | 576 | O | GLN | A1076 | 17.000 | 51.249 | 22.116 | 1.00 | 34.87 | O | |
| ATOM | 577 | N | ASP | A1077 | 14.840 | 50.638 | 22.202 | 1.00 | 33.69 | N | |
| ATOM | 578 | CA | ASP | A1077 | 14.838 | 50.395 | 23.641 | 1.00 | 32.88 | C | |
| ATOM | 579 | CB | ASP | A1077 | 13.466 | 50.738 | 24.229 | 1.00 | 34.04 | C | |
| ATOM | 580 | CG | ASP | A1077 | 13.183 | 52.230 | 24.220 | 1.00 | 35.48 | C | |
| ATOM | 581 | OD1 | ASP | A1077 | 13.877 | 52.971 | 24.953 | 1.00 | 36.31 | O | |
| ATOM | 582 | OD2 | ASP | A1077 | 12.269 | 52.659 | 23.477 | 1.00 | 36.39 | O | |
| ATOM | 583 | C | ASP | A1077 | 15.170 | 48.935 | 23.943 | 1.00 | 31.37 | C | |
| ATOM | 584 | O | ASP | A1077 | 15.205 | 48.517 | 25.103 | 1.00 | 30.93 | O | |
| ATOM | 585 | N | LEU | A1078 | 15.408 | 48.165 | 22.889 | 1.00 | 29.66 | N | |
| ATOM | 586 | CA | LEU | A1078 | 15.724 | 46.753 | 23.026 | 1.00 | 28.12 | C | |
| ATOM | 587 | CB | LEU | A1078 | 14.605 | 45.907 | 22.410 | 1.00 | 28.40 | C | |
| ATOM | 588 | CG | LEU | A1078 | 13.211 | 46.138 | 22.999 | 1.00 | 28.65 | C | |
| ATOM | 589 | CD1 | LEU | A1078 | 12.175 | 45.297 | 22.250 | 1.00 | 28.42 | C | |
| ATOM | 590 | CD2 | LEU | A1078 | 13.232 | 45.784 | 24.485 | 1.00 | 28.42 | C | |
| ATOM | 591 | C | LEU | A1078 | 17.029 | 46.469 | 22.317 | 1.00 | 26.77 | C | |
| ATOM | 592 | O | LEU | A1078 | 17.256 | 46.963 | 21.219 | 1.00 | 26.91 | O | |
| ATOM | 593 | N | GLU | A1079 | 17.892 | 45.683 | 22.944 | 1.00 | 25.48 | N | |
| ATOM | 594 | CA | GLU | A1079 | 19.166 | 45.352 | 22.330 | 1.00 | 24.50 | C | |
| ATOM | 595 | CB | GLU | A1079 | 20.287 | 46.202 | 22.921 | 1.00 | 24.22 | C | |
| ATOM | 596 | CG | GLU | A1079 | 21.561 | 46.128 | 22.114 | 1.00 | 23.64 | C | |
| ATOM | 597 | CD | GLU | A1079 | 22.638 | 47.051 | 22.635 | 1.00 | 24.07 | C | |
| ATOM | 598 | QE1 | GLU | A1079 | 23.079 | 46.862 | 23.791 | 1.00 | 23.41 | O | |
| ATOM | 599 | OE2 | GLU | A1079 | 23.044 | 47.967 | 21.883 | 1.00 | 24.29 | O | |
| ATOM | 600 | C | GLU | A1079 | 19.512 | 43.885 | 22.488 | 1.00 | 23.86 | C | |
| ATOM | 601 | O | GLU | A1079 | 19.341 | 43.305 | 23.560 | 1.00 | 23.77 | O | |
| ATOM | 602 | N | VAL | A1080 | 20.003 | 43.291 | 21.408 | 1.00 | 23.09 | N | |
| ATOM | 603 | CA | VAL | A1080 | 20.377 | 41.888 | 21.411 | 1.00 | 22.78 | C | |
| ATOM | 604 | CB | VAL | A1080 | 19.413 | 41.032 | 20.529 | 1.00 | 22.41 | C | |
| ATOM | 605 | CG1 | VAL | A1080 | 20.005 | 39.649 | 20.284 | 1.00 | 21.10 | C | |
| ATOM | 606 | CG2 | VAL | A1080 | 18.059 | 40.893 | 21.224 | 1.00 | 21.12 | C | |
| ATOM | 607 | C | VAL | A1080 | 21.800 | 41.707 | 20.909 | 1.00 | 23.31 | C | |
| ATOM | 608 | O | VAL | A1080 | 22.102 | 41.942 | 19.737 | 1.00 | 23.36 | O | |
| ATOM | 609 | N | LEU | A1081 | 22.673 | 41.292 | 21.818 | 1.00 | 23.52 | N | |
| ATOM | 610 | CA | LEU | A1081 | 24.066 | 41.056 | 21.488 | 1.00 | 23.32 | C | |
| ATOM | 611 | CB | LEU | A1081 | 24.968 | 41.910 | 22.391 | 1.00 | 22.81 | C | |
| ATOM | 612 | CG | LEU | A1081 | 24.725 | 43.426 | 22.313 | 1.00 | 22.95 | C | |
| ATOM | 613 | CD1 | LEU | A1081 | 25.680 | 44.170 | 23.239 | 1.00 | 22.31 | C | |
| ATOM | 614 | CD2 | LEU | A1081 | 24.919 | 43.902 | 20.874 | 1.00 | 23.08 | C | |
| ATOM | 615 | C | LEU | A1081 | 24.347 | 39.576 | 21.709 | 1.00 | 23.58 | C | |
| ATOM | 616 | O | LEU | A1081 | 23.436 | 38.786 | 21.981 | 1.00 | 22.36 | O | |
| ATOM | 617 | N | ASN | A1082 | 25.614 | 39.204 | 21.582 | 1.00 | 24.47 | N | |
| ATOM | 618 | CA | ASN | A1082 | 26.018 | 37.826 | 21.802 | 1.00 | 25.10 | C | |
| ATOM | 619 | CB | ASN | A1082 | 26.745 | 37.301 | 20.564 | 1.00 | 25.30 | C | |
| ATOM | 620 | CG | ASN | A1082 | 25.798 | 37.072 | 19.406 | 1.00 | 25.42 | C | |
| ATOM | 621 | OD1 | ASN | A1082 | 24.863 | 36.277 | 19.514 | 1.00 | 26.33 | O | |
| ATOM | 622 | ND2 | ASN | A1082 | 26.023 | 37.769 | 18.299 | 1.00 | 25.02 | N | |
| ATOM | 623 | C | ASN | A1082 | 26.897 | 37.774 | 23.042 | 1.00 | 25.09 | C | |
| ATOM | 624 | O | ASN | A1082 | 27.802 | 38.586 | 23.197 | 1.00 | 24.45 | O | |
| ATOM | 625 | N | ASN | A1083 | 26.614 | 36.825 | 23.932 | 1.00 | 26.16 | N | |
| ATOM | 626 | CA | ASN | A1083 | 27.365 | 36.697 | 25.180 | 1.00 | 26.89 | C | |
| ATOM | 627 | CB | ASN | A1083 | 26.484 | 36.048 | 26.264 | 1.00 | 26.14 | C | |
| ATOM | 628 | CG | ASN | A1083 | 26.094 | 34.600 | 25.952 | 1.00 | 25.65 | C | |
| ATOM | 629 | OD1 | ASN | A1083 | 25.289 | 34.006 | 26.668 | 1.00 | 25.06 | O | |
| ATOM | 630 | ND2 | ASN | A1083 | 26.665 | 34.032 | 24.900 | 1.00 | 25.04 | N | |
| ATOM | 631 | C | ASN | A1083 | 28.691 | 35.953 | 25.044 | 1.00 | 27.81 | C | |
| ATOM | 632 | O | ASN | A1083 | 29.098 | 35.595 | 23.939 | 1.00 | 27.57 | O | |
| ATOM | 633 | N | GLU | A1084 | 29.373 | 35.729 | 26.162 | 1.00 | 29.55 | N | |
| ATOM | 634 | CA | GLU | A1084 | 30.656 | 35.041 | 26.102 | 1.00 | 31.72 | C | |
| ATOM | 635 | CB | GLU | A1084 | 31.382 | 35.091 | 27.465 | 1.00 | 32.66 | C | |
| ATOM | 636 | CG | GLU | A1084 | 30.618 | 34.574 | 28.685 | 1.00 | 34.91 | C | |
| ATOM | 637 | CD | GLU | A1084 | 31.407 | 34.752 | 29.998 | 1.00 | 36.48 | C | |
| ATOM | 638 | OE1 | GLU | A1084 | 32.421 | 34.051 | 30.192 | 1.00 | 37.32 | O | |
| ATOM | 639 | OE2 | GLU | A1084 | 31.022 | 35.601 | 30.841 | 1.00 | 37.46 | O | |
| ATOM | 640 | C | GLU | A1084 | 30.532 | 33.603 | 25.584 | 1.00 | 32.13 | C | |
| ATOM | 641 | O | GLU | A1084 | 31.522 | 33.007 | 25.186 | 1.00 | 32.93 | O | |
| ATOM | 642 | N | ARG | A1085 | 29.322 | 33.052 | 25.578 | 1.00 | 32.33 | N | |
| ATOM | 643 | CA | ARG | A1085 | 29.118 | 31.703 | 25.061 | 1.00 | 32.71 | C | |
| ATOM | 644 | CB | ARG | A1085 | 28.057 | 30.950 | 25.866 | 1.00 | 34.65 | C | |
| ATOM | 645 | CG | ARG | A1085 | 28.525 | 30.506 | 27.238 | 1.00 | 37.26 | C | |
| ATOM | 646 | CD | ARG | A1085 | 29.588 | 29.432 | 27.112 | 1.00 | 39.70 | C | |
| ATOM | 647 | NE | ARG | A1085 | 30.430 | 29.337 | 28.303 | 1.00 | 42.39 | N | |
| ATOM | 648 | CZ | ARG | A1085 | 31.329 | 28.375 | 28.501 | 1.00 | 43.44 | C | |
| ATOM | 649 | NH1 | ARG | A1085 | 32.062 | 28.363 | 29.611 | 1.00 | 44.05 | N | |
| ATOM | 650 | NH2 | ARG | A1085 | 31.482 | 27.415 | 27.593 | 1.00 | 43.73 | N | |
| ATOM | 651 | C | ARG | A1085 | 28.682 | 31.788 | 23.606 | 1.00 | 31.86 | C | |
| ATOM | 652 | O | ARG | A1085 | 28.369 | 30.775 | 22.983 | 1.00 | 32.12 | O | |
| ATOM | 653 | N | GLY | A1086 | 28.664 | 33.006 | 23.070 | 1.00 | 30.59 | N | |
| ATOM | 654 | CA | GLY | A1086 | 28.275 | 33.206 | 21.687 | 1.00 | 29.15 | C | |
| ATOM | 655 | C | GLY | A1086 | 26.779 | 33.208 | 21.427 | 1.00 | 28.22 | C | |
| ATOM | 656 | O | GLY | A1086 | 26.347 | 33.336 | 20.287 | 1.00 | 28.76 | O | |
| ATOM | 657 | N | ALA | A1087 | 25.980 | 33.079 | 22.476 | 1.00 | 26.78 | N | |
| ATOM | 658 | CA | ALA | A1087 | 24.536 | 33.052 | 22.313 | 1.00 | 25.40 | C | |
| ATOM | 659 | CB | ALA | A1087 | 23.907 | 32.162 | 23.378 | 1.00 | 24.76 | C | |
| ATOM | 660 | C | ALA | A1087 | 23.936 | 34.448 | 22.390 | 1.00 | 24.84 | C | |
| ATOM | 661 | O | ALA | A1087 | 24.417 | 35.304 | 23.142 | 1.00 | 24.29 | O | |
| ATOM | 662 | N | PRO | A1088 | 22.886 | 34.703 | 21.591 | 1.00 | 23.94 | N | |
| ATOM | 663 | CD | PRO | A1088 | 22.363 | 33.864 | 20.496 | 1.00 | 23.58 | C | |
| ATOM | 664 | CA | PRO | A1088 | 22.234 | 36.009 | 21.601 | 1.00 | 23.19 | C | |
| ATOM | 665 | CB | PRO | A1088 | 21.303 | 35.944 | 20.388 | 1.00 | 23.32 | C | |
| ATOM | 666 | CG | PRO | A1088 | 21.019 | 34.482 | 20.239 | 1.00 | 23.28 | C | |
| ATOM | 667 | C | PRO | A1088 | 21.483 | 36.232 | 22.911 | 1.00 | 22.65 | C | |
| ATOM | 668 | O | PRO | A1088 | 20.908 | 35.304 | 23.475 | 1.00 | 22.59 | O | |
| ATOM | 669 | N | TYR | A1089 | 21.518 | 37.464 | 23.407 | 1.00 | 22.22 | N | |
| ATOM | 670 | CA | TYR | A1089 | 20.824 | 37.808 | 24.645 | 1.00 | 21.73 | C | |
| ATOM | 671 | CB | TYR | A1089 | 21.720 | 37.537 | 25.866 | 1.00 | 21.31 | C | |
| ATOM | 672 | CG | TYR | A1089 | 22.843 | 38.540 | 26.058 | 1.00 | 20.82 | C | |
| ATOM | 673 | CD1 | TYR | A1089 | 22.795 | 39.475 | 27.095 | 1.00 | 20.39 | C | |
| ATOM | 674 | CE1 | TYR | A1089 | 23.791 | 40.428 | 27.253 | 1.00 | 20.46 | C | |
| ATOM | 675 | CD2 | TYR | A1089 | 23.931 | 38.582 | 25.177 | 1.00 | 20.50 | C | |
| ATOM | 676 | CE2 | TYR | A1089 | 24.942 | 39.535 | 25.323 | 1.00 | 20.31 | C | |
| ATOM | 677 | CZ | TYR | A1089 | 24.865 | 40.458 | 26.363 | 1.00 | 21.12 | C | |
| ATOM | 678 | OH | TYR | A1089 | 25.844 | 41.421 | 26.507 | 1.00 | 20.91 | O | |
| ATOM | 679 | C | TYR | A1089 | 20.459 | 39.278 | 24.598 | 1.00 | 21.43 | C | |
| ATOM | 680 | O | TYR | A1089 | 20.986 | 40.045 | 23.782 | 1.00 | 21.20 | O | |
| ATOM | 681 | N | PHE | A1090 | 19.541 | 39.670 | 25.469 | 1.00 | 21.81 | N | |
| ATOM | 682 | CA | PHE | A1090 | 19.134 | 41.058 | 25.534 | 1.00 | 21.85 | C | |
| ATOM | 683 | CB | PHE | A1090 | 17.694 | 41.182 | 26.041 | 1.00 | 20.99 | C | |
| ATOM | 684 | CG | PHE | A1090 | 16.651 | 40.841 | 25.006 | 1.00 | 20.79 | C | |
| ATOM | 685 | CD1 | PHE | A1090 | 16.294 | 39.519 | 24.755 | 1.00 | 20.19 | C | |
| ATOM | 686 | CD2 | PHE | A1090 | 16.052 | 41.849 | 24.247 | 1.00 | 20.29 | C | |
| ATOM | 687 | CE1 | PHE | A1090 | 15.364 | 39.207 | 23.760 | 1.00 | 19.79 | C | |
| ATOM | 688 | CE2 | PHE | A1090 | 15.122 | 41.543 | 23.252 | 1.00 | 20.00 | C | |
| ATOM | 689 | CZ | PHE | A1090 | 14.778 | 40.221 | 23.007 | 1.00 | 19.21 | C | |
| ATOM | 690 | C | PHE | A1090 | 20.087 | 41.794 | 26.467 | 1.00 | 22.39 | C | |
| ATOM | 691 | O | PHE | A1090 | 20.197 | 41.460 | 27.643 | 1.00 | 22.82 | O | |
| ATOM | 692 | N | SER | A1091 | 20.791 | 42.780 | 25.922 | 1.00 | 23.17 | N | |
| ATOM | 693 | CA | SER | A1091 | 21.725 | 43.590 | 26.697 | 1.00 | 24.38 | C | |
| ATOM | 694 | CB | SER | A1091 | 22.879 | 44.048 | 25.805 | 1.00 | 24.43 | C | |
| ATOM | 695 | OG | SER | A1091 | 22.405 | 44.352 | 24.504 | 1.00 | 25.11 | O | |
| ATOM | 696 | C | SER | A1091 | 20.979 | 44.805 | 27.248 | 1.00 | 24.91 | C | |
| ATOM | 697 | O | SER | A1091 | 21.420 | 45.443 | 28.204 | 1.00 | 24.93 | O | |
| ATOM | 698 | N | GLN | A1092 | 19.854 | 45.124 | 26.618 | 1.00 | 25.07 | N | |
| ATOM | 699 | CA | GLN | A1092 | 19.023 | 46.235 | 27.046 | 1.00 | 26.14 | C | |
| ATOM | 700 | CB | GLN | A1092 | 19.357 | 47.517 | 26.258 | 1.00 | 27.41 | C | |
| ATOM | 701 | CG | GLN | A1092 | 20.779 | 48.089 | 26.463 | 1.00 | 29.10 | C | |
| ATOM | 702 | CD | GLN | A1092 | 21.096 | 48.475 | 27.918 | 1.00 | 31.36 | C | |
| ATOM | 703 | QE1 | GLN | A1092 | 20.273 | 49.093 | 28.607 | 1.00 | 32.18 | O | |
| ATOM | 704 | NE2 | GLN | A1092 | 22.306 | 48.127 | 28.380 | 1.00 | 31.33 | N | |
| ATOM | 705 | C | GLN | A1092 | 17.556 | 45.850 | 26.841 | 1.00 | 25.78 | C | |
| ATOM | 706 | O | GLN | A1092 | 17.148 | 45.443 | 25.753 | 1.00 | 25.27 | O | |
| ATOM | 707 | N | ALA | A1093 | 16.777 | 45.961 | 27.909 | 1.00 | 25.60 | N | |
| ATOM | 708 | CA | ALA | A1093 | 15.357 | 45.630 | 27.870 | 1.00 | 25.62 | C | |
| ATOM | 709 | CB | ALA | A1093 | 15.171 | 44.110 | 27.867 | 1.00 | 24.75 | C | |
| ATOM | 710 | C | ALA | A1093 | 14.677 | 46.238 | 29.090 | 1.00 | 25.61 | C | |
| ATOM | 711 | O | ALA | A1093 | 15.220 | 46.209 | 30.186 | 1.00 | 25.48 | O | |
| ATOM | 712 | N | PRO | A1094 | 13.474 | 46.796 | 28.916 | 1.00 | 26.29 | N | |
| ATOM | 713 | CD | PRO | A1094 | 12.668 | 46.878 | 27.682 | 1.00 | 26.31 | C | |
| ATOM | 714 | CA | PRO | A1094 | 12.758 | 47.400 | 30.046 | 1.00 | 26.85 | C | |
| ATOM | 715 | CB | PRO | A1094 | 11.622 | 48.147 | 29.358 | 1.00 | 26.71 | C | |
| ATOM | 716 | CG | PRO | A1094 | 11.292 | 47.223 | 28.222 | 1.00 | 26.01 | C | |
| ATOM | 717 | C | PRO | A1094 | 12.241 | 46.343 | 31.024 | 1.00 | 27.25 | C | |
| ATOM | 718 | O | PRO | A1094 | 11.182 | 46.511 | 31.631 | 1.00 | 28.16 | O | |
| ATOM | 719 | N | PHE | A1095 | 12.980 | 45.248 | 31.167 | 1.00 | 26.92 | N | |
| ATOM | 720 | CA | PHE | A1095 | 12.571 | 44.181 | 32.069 | 1.00 | 26.96 | C | |
| ATOM | 721 | CB | PHE | A1095 | 12.234 | 42.918 | 31.279 | 1.00 | 26.69 | C | |
| ATOM | 722 | CG | PHE | A1095 | 11.729 | 41.782 | 32.127 | 1.00 | 26.67 | C | |
| ATOM | 723 | CD1 | PHE | A1095 | 12.396 | 40.553 | 32.137 | 1.00 | 26.30 | C | |
| ATOM | 724 | CD2 | PHE | A1095 | 10.567 | 41.920 | 32.879 | 1.00 | 25.87 | C | |
| ATOM | 725 | CE1 | PHE | A1095 | 11.911 | 39.481 | 32.878 | 1.00 | 25.90 | C | |
| ATOM | 726 | CE2 | PHE | A1095 | 10.074 | 40.853 | 33.624 | 1.00 | 25.69 | C | |
| ATOM | 727 | CZ | PHE | A1095 | 10.746 | 39.628 | 33.625 | 1.00 | 26.13 | C | |
| ATOM | 728 | C | PHE | A1095 | 13.685 | 43.891 | 33.055 | 1.00 | 26.99 | C | |
| ATOM | 729 | O | PHE | A1095 | 14.854 | 43.835 | 32.680 | 1.00 | 27.34 | O | |
| ATOM | 730 | N | SER | A1096 | 13.319 | 43.687 | 34.314 | 1.00 | 26.53 | N | |
| ATOM | 731 | CA | SER | A1096 | 14.308 | 43.436 | 35.340 | 1.00 | 26.73 | C | |
| ATOM | 732 | CB | SER | A1096 | 13.973 | 44.263 | 36.596 | 1.00 | 27.70 | C | |
| ATOM | 733 | OG | SER | A1096 | 12.642 | 44.023 | 37.052 | 1.00 | 29.18 | O | |
| ATOM | 734 | C | SER | A1096 | 14.500 | 41.968 | 35.715 | 1.00 | 26.21 | C | |
| ATOM | 735 | O | SER | A1096 | 15.426 | 41.639 | 36.464 | 1.00 | 26.51 | O | |
| ATOM | 736 | N | GLY | A1097 | 13.648 | 41.088 | 35.198 | 1.00 | 25.26 | N | |
| ATOM | 737 | CA | GLY | A1097 | 13.773 | 39.679 | 35.521 | 1.00 | 24.11 | C | |
| ATOM | 738 | C | GLY | A1097 | 14.633 | 38.918 | 34.529 | 1.00 | 24.17 | C | |
| ATOM | 739 | O | GLY | A1097 | 15.383 | 39.512 | 33.758 | 1.00 | 24.01 | O | |
| ATOM | 740 | N | LYS | A1098 | 14.526 | 37.594 | 34.552 | 1.00 | 23.74 | N | |
| ATOM | 741 | CA | LYS | A1098 | 15.281 | 36.745 | 33.643 | 1.00 | 23.42 | C | |
| ATOM | 742 | CB | LYS | A1098 | 15.360 | 35.309 | 34.172 | 1.00 | 24.51 | C | |
| ATOM | 743 | CG | LYS | A1098 | 16.177 | 35.141 | 35.444 | 1.00 | 26.65 | C | |
| ATOM | 744 | CD | LYS | A1098 | 16.236 | 33.682 | 35.898 | 1.00 | 28.23 | C | |
| ATOM | 745 | CE | LYS | A1098 | 17.224 | 33.510 | 37.058 | 1.00 | 30.02 | C | |
| ATOM | 746 | NZ | LYS | A1098 | 17.270 | 32.108 | 37.594 | 1.00 | 30.95 | N | |
| ATOM | 747 | C | LYS | A1098 | 14.602 | 36.730 | 32.287 | 1.00 | 22.48 | C | |
| ATOM | 748 | O | LYS | A1098 | 13.374 | 36.671 | 32.192 | 1.00 | 22.17 | O | |
| ATOM | 749 | N | ILE | A1099 | 15.419 | 36.782 | 31.241 | 1.00 | 21.41 | N | |
| ATOM | 750 | CA | ILE | A1099 | 14.941 | 36.761 | 29.876 | 1.00 | 20.15 | C | |
| ATOM | 751 | CB | ILE | A1099 | 15.333 | 38.039 | 29.134 | 1.00 | 19.84 | C | |
| ATOM | 752 | CG2 | ILE | A1099 | 14.804 | 37.989 | 27.713 | 1.00 | 20.13 | C | |
| ATOM | 753 | CG1 | ILE | A1099 | 14.791 | 39.263 | 29.873 | 1.00 | 19.76 | C | |
| ATOM | 754 | CD1 | ILE | A1099 | 15.218 | 40.583 | 29.262 | 1.00 | 18.64 | C | |
| ATOM | 755 | C | ILE | A1099 | 15.576 | 35.581 | 29.148 | 1.00 | 19.81 | C | |
| ATOM | 756 | O | ILE | A1099 | 16.791 | 35.528 | 28.972 | 1.00 | 20.43 | O | |
| ATOM | 757 | N | TRP | A1100 | 14.761 | 34.623 | 28.740 | 1.00 | 19.20 | N | |
| ATOM | 758 | CA | TRP | A1100 | 15.277 | 33.480 | 28.005 | 1.00 | 18.42 | C | |
| ATOM | 759 | CB | TRP | A1100 | 14.653 | 32.193 | 28.530 | 1.00 | 17.66 | C | |
| ATOM | 760 | CG | TRP | A1100 | 14.970 | 31.962 | 29.973 | 1.00 | 17.80 | C | |
| ATOM | 761 | CD2 | TRP | A1100 | 16.150 | 31.351 | 30.501 | 1.00 | 17.39 | C | |
| ATOM | 762 | CE2 | TRP | A1100 | 16.038 | 31.375 | 31.914 | 1.00 | 17.58 | C | |
| ATOM | 763 | CE3 | TRP | A1100 | 17.294 | 30.787 | 29.921 | 1.00 | 17.40 | C | |
| ATOM | 764 | CD1 | TRP | A1100 | 14.210 | 32.323 | 31.055 | 1.00 | 17.92 | C | |
| ATOM | 765 | NE1 | TRP | A1100 | 14.844 | 31.972 | 32.222 | 1.00 | 17.09 | N | |
| ATOM | 766 | CZ2 | TRP | A1100 | 17.030 | 30.851 | 32.759 | 1.00 | 16.77 | C | |
| ATOM | 767 | CZ3 | TRP | A1100 | 18.283 | 30.268 | 30.763 | 1.00 | 17.50 | C | |
| ATOM | 768 | CH2 | TRP | A1100 | 18.138 | 30.307 | 32.167 | 1.00 | 17.07 | C | |
| ATOM | 769 | C | TRP | A1100 | 14.946 | 33.700 | 26.526 | 1.00 | 17.86 | C | |
| ATOM | 770 | O | TRP | A1100 | 13.791 | 33.866 | 26.143 | 1.00 | 18.05 | O | |
| ATOM | 771 | N | LEU | A1100 | 15.987 | 33.725 | 25.707 | 1.00 | 17.46 | N | |
| ATOM | 772 | CA | LEU | A1100 | 15.852 | 33.967 | 24.290 | 1.00 | 16.38 | C | |
| ATOM | 773 | CB | LEU | A1100 | 16.469 | 35.325 | 23.955 | 1.00 | 15.64 | C | |
| ATOM | 774 | CG | LEU | A1100 | 16.715 | 35.601 | 22.468 | 1.00 | 15.15 | C | |
| ATOM | 775 | CD1 | LEU | A1100 | 15.379 | 35.873 | 21.762 | 1.00 | 13.79 | C | |
| ATOM | 776 | CD2 | LEU | A1100 | 17.669 | 36.775 | 22.317 | 1.00 | 14.27 | C | |
| ATOM | 777 | C | LEU | A1100 | 16.520 | 32.916 | 23.429 | 1.00 | 16.47 | C | |
| ATOM | 778 | O | LEU | A1100 | 17.624 | 32.474 | 23.720 | 1.00 | 16.38 | O | |
| ATOM | 779 | N | SER | A1102 | 15.847 | 32.520 | 22.356 | 1.00 | 17.12 | N | |
| ATOM | 780 | CA | SER | A1102 | 16.429 | 31.565 | 21.432 | 1.00 | 17.39 | C | |
| ATOM | 781 | CB | SER | A1102 | 15.962 | 30.140 | 21.722 | 1.00 | 16.65 | C | |
| ATOM | 782 | OG | SER | A1102 | 16.736 | 29.217 | 20.966 | 1.00 | 15.28 | O | |
| ATOM | 783 | C | SER | A1102 | 16.052 | 31.965 | 20.011 | 1.00 | 18.55 | C | |
| ATOM | 784 | O | SER | A1102 | 14.902 | 32.319 | 19.727 | 1.00 | 18.33 | O | |
| ATOM | 785 | N | ILE | A1103 | 17.043 | 31.930 | 19.127 | 1.00 | 19.20 | N | |
| ATOM | 786 | CA | ILE | A1103 | 16.836 | 32.286 | 17.740 | 1.00 | 20.42 | C | |
| ATOM | 787 | CB | ILE | A1103 | 17.588 | 33.578 | 17.386 | 1.00 | 20.38 | C | |
| ATOM | 788 | CG2 | ILE | A1103 | 17.216 | 34.026 | 15.981 | 1.00 | 19.83 | C | |
| ATOM | 789 | CG1 | ILE | A1103 | 17.240 | 34.675 | 18.399 | 1.00 | 20.72 | C | |
| ATOM | 790 | CD1 | ILE | A1103 | 17.996 | 35.980 | 18.172 | 1.00 | 20.84 | C | |
| ATOM | 791 | C | ILE | A1103 | 17.347 | 31.169 | 16.843 | 1.00 | 21.44 | C | |
| ATOM | 792 | O | ILE | A1103 | 18.373 | 30.551 | 17.121 | 1.00 | 21.72 | O | |
| ATOM | 793 | N | SER | A1104 | 16.625 | 30.916 | 15.764 | 1.00 | 21.94 | N | |
| ATOM | 794 | CA | SER | A1104 | 17.015 | 29.884 | 14.823 | 1.00 | 22.99 | C | |
| ATOM | 795 | CB | SER | A1104 | 16.334 | 28.556 | 15.185 | 1.00 | 22.73 | C | |
| ATOM | 796 | OG | SER | A1104 | 16.618 | 27.550 | 14.229 | 1.00 | 21.97 | O | |
| ATOM | 797 | C | SER | A1104 | 16.607 | 30.322 | 13.421 | 1.00 | 23.89 | C | |
| ATOM | 798 | O | SER | A1104 | 15.698 | 31.139 | 13.246 | 1.00 | 24.13 | O | |
| ATOM | 799 | N | HIS | A1100 | 17.283 | 29.787 | 12.417 | 1.00 | 25.15 | N | |
| ATOM | 800 | CA | HIS | A1100 | 16.943 | 30.144 | 11.049 | 1.00 | 26.29 | C | |
| ATOM | 801 | CB | HIS | A1100 | 17.665 | 31.429 | 10.633 | 1.00 | 26.35 | C | |
| ATOM | 802 | CG | HIS | A1100 | 19.153 | 31.290 | 10.559 | 1.00 | 27.08 | C | |
| ATOM | 803 | CD2 | HIS | A1100 | 19.960 | 30.889 | 9.549 | 1.00 | 27.02 | C | |
| ATOM | 804 | ND1 | HIS | A1100 | 19.980 | 31.551 | 11.630 | 1.00 | 27.58 | N | |
| ATOM | 805 | CE1 | HIS | A1100 | 21.234 | 31.317 | 11.283 | 1.00 | 27.17 | C | |
| ATOM | 806 | NE2 | HIS | A1100 | 21.249 | 30.914 | 10.025 | 1.00 | 27.22 | N | |
| ATOM | 807 | C | HIS | A1100 | 17.276 | 29.060 | 10.042 | 1.00 | 26.76 | C | |
| ATOM | 808 | O | HIS | A1100 | 18.031 | 28.129 | 10.318 | 1.00 | 26.55 | O | |
| ATOM | 809 | N | THR | A1106 | 16.670 | 29.197 | 8.874 | 1.00 | 27.49 | N | |
| ATOM | 810 | CA | THR | A1106 | 16.901 | 28.309 | 7.751 | 1.00 | 28.62 | C | |
| ATOM | 811 | CB | THR | A1106 | 15.595 | 27.643 | 7.273 | 1.00 | 28.44 | C | |
| ATOM | 812 | OG1 | THR | A1106 | 14.721 | 28.635 | 6.712 | 1.00 | 27.15 | O | |
| ATOM | 813 | CG2 | THR | A1106 | 14.901 | 26.954 | 8.447 | 1.00 | 28.62 | C | |
| ATOM | 814 | C | THR | A1106 | 17.381 | 29.299 | 6.698 | 1.00 | 29.33 | C | |
| ATOM | 815 | O | THR | A1106 | 17.685 | 30.442 | 7.027 | 1.00 | 29.14 | O | |
| ATOM | 816 | N | ASP | A1107 | 17.446 | 28.880 | 5.444 | 1.00 | 30.59 | N | |
| ATOM | 817 | CA | ASP | A1107 | 17.878 | 29.789 | 4.392 | 1.00 | 31.74 | C | |
| ATOM | 818 | CB | ASP | A1107 | 18.324 | 28.992 | 3.160 | 1.00 | 33.44 | C | |
| ATOM | 819 | CG | ASP | A1107 | 19.499 | 28.054 | 3.454 | 1.00 | 35.18 | C | |
| ATOM | 820 | OD1 | ASP | A1107 | 19.827 | 27.214 | 2.582 | 1.00 | 36.15 | O | |
| ATOM | 821 | OD2 | ASP | A1107 | 20.099 | 28.153 | 4.548 | 1.00 | 35.79 | O | |
| ATOM | 822 | C | ASP | A1107 | 16.720 | 30.722 | 4.020 | 1.00 | 31.63 | C | |
| ATOM | 823 | O | ASP | A1107 | 16.913 | 31.724 | 3.334 | 1.00 | 31.90 | O | |
| ATOM | 824 | N | GLN | A1100 | 15.518 | 30.401 | 4.494 | 1.00 | 31.41 | N | |
| ATOM | 825 | CA | GLN | A1108 | 14.336 | 31.201 | 4.175 | 1.00 | 30.98 | C | |
| ATOM | 826 | CB | GLN | A1108 | 13.340 | 30.345 | 3.399 | 1.00 | 32.64 | C | |
| ATOM | 827 | CG | GLN | A1108 | 13.860 | 29.898 | 2.038 | 1.00 | 34.63 | C | |
| ATOM | 828 | CD | GLN | A1108 | 12.917 | 28.945 | 1.346 | 1.00 | 35.98 | C | |
| ATOM | 829 | OE1 | GLN | A1108 | 12.698 | 27.819 | 1.807 | 1.00 | 36.85 | O | |
| ATOM | 830 | NE2 | GLN | A1108 | 12.340 | 29.391 | 0.231 | 1.00 | 36.81 | N | |
| ATOM | 831 | C | GLN | A1108 | 13.616 | 31.862 | 5.338 | 1.00 | 29.94 | C | |
| ATOM | 832 | O | GLN | A1100 | 12.985 | 32.899 | 5.158 | 1.00 | 29.44 | O | |
| ATOM | 833 | N | PHE | A1109 | 13.700 | 31.269 | 6.529 | 1.00 | 29.06 | N | |
| ATOM | 834 | CA | PHE | A1109 | 13.024 | 31.838 | 7.692 | 1.00 | 27.73 | C | |
| ATOM | 835 | CB | PHE | A1109 | 11.762 | 31.036 | 8.037 | 1.00 | 27.57 | C | |
| ATOM | 836 | CG | PHE | A1109 | 10.744 | 31.005 | 6.941 | 1.00 | 27.23 | C | |
| ATOM | 837 | CD1 | PHE | A1109 | 10.788 | 30.017 | 5.962 | 1.00 | 27.53 | C | |
| ATOM | 838 | CD2 | PHE | A1109 | 9.749 | 31.976 | 6.871 | 1.00 | 27.16 | C | |
| ATOM | 839 | CE1 | PHE | A1109 | 9.852 | 30.000 | 4.921 | 1.00 | 27.28 | C | |
| ATOM | 840 | CE2 | PHE | A1109 | 8.810 | 31.970 | 5.838 | 1.00 | 26.77 | C | |
| ATOM | 841 | CZ | PHE | A1109 | 8.861 | 30.981 | 4.861 | 1.00 | 26.97 | C | |
| ATOM | 842 | C | PHE | A1109 | 13.868 | 31.932 | 8.950 | 1.00 | 26.95 | C | |
| ATOM | 843 | O | PHE | A1109 | 14.877 | 31.243 | 9.102 | 1.00 | 27.19 | O | |
| ATOM | 844 | N | VAL | A1110 | 13.431 | 32.803 | 9.852 | 1.00 | 25.89 | N | |
| ATOM | 845 | CA | VAL | A1110 | 14.080 | 32.992 | 11.138 | 1.00 | 25.13 | C | |
| ATOM | 846 | CB | VAL | A1110 | 14.844 | 34.347 | 11.197 | 1.00 | 25.40 | C | |
| ATOM | 847 | CG1 | VAL | A1110 | 13.874 | 35.510 | 11.081 | 1.00 | 25.64 | C | |
| ATOM | 848 | CG2 | VAL | A1110 | 15.623 | 34.449 | 12.499 | 1.00 | 25.77 | C | |
| ATOM | 849 | C | VAL | A1110 | 12.956 | 32.973 | 12.184 | 1.00 | 24.39 | C | |
| ATOM | 850 | O | VAL | A1110 | 11.863 | 33.500 | 11.936 | 1.00 | 24.17 | O | |
| ATOM | 851 | N | THR | A1111 | 13.206 | 32.344 | 13.332 | 1.00 | 23.30 | N | |
| ATOM | 852 | CA | THR | A1111 | 12.208 | 32.289 | 14.404 | 1.00 | 22.55 | C | |
| ATOM | 853 | CB | THR | A1111 | 11.610 | 30.888 | 14.562 | 1.00 | 22.93 | C | |
| ATOM | 854 | OG1 | THR | A1111 | 12.670 | 29.954 | 14.786 | 1.00 | 24.79 | O | |
| ATOM | 855 | CG2 | THR | A1111 | 10.839 | 30.479 | 13.323 | 1.00 | 23.61 | C | |
| ATOM | 856 | C | THR | A1111 | 12.830 | 32.664 | 15.746 | 1.00 | 21.62 | C | |
| ATOM | 857 | O | THR | A1111 | 13.999 | 32.377 | 16.002 | 1.00 | 21.64 | O | |
| ATOM | 858 | N | ALA | A1112 | 12.041 | 33.299 | 16.605 | 1.00 | 20.37 | N | |
| ATOM | 859 | CA | ALA | A1112 | 12.522 | 33.688 | 17.922 | 1.00 | 19.66 | C | |
| ATOM | 860 | CB | ALA | A1112 | 12.800 | 35.202 | 17.970 | 1.00 | 19.55 | C | |
| ATOM | 861 | C | ALA | A1112 | 11.516 | 33.311 | 19.004 | 1.00 | 19.07 | C | |
| ATOM | 862 | O | ALA | A1112 | 10.302 | 33.410 | 18.814 | 1.00 | 19.29 | O | |
| ATOM | 863 | N | SER | A1113 | 12.034 | 32.872 | 20.139 | 1.00 | 17.99 | N | |
| ATOM | 864 | CA | SER | A1113 | 11.195 | 32.506 | 21.263 | 1.00 | 17.22 | C | |
| ATOM | 865 | CB | SER | A1113 | 11.174 | 30;989 | 21.457 | 1.00 | 16.88 | C | |
| ATOM | 866 | OG | SER | A1113 | 10.313 | 30.614 | 22.521 | 1.00 | 16.26 | O | |
| ATOM | 867 | C | SER | A1113 | 11.782 | 33.180 | 22.487 | 1.00 | 17.09 | C | |
| ATOM | 868 | O | SER | A1113 | 12.995 | 33.112 | 22.733 | 1.00 | 16.32 | O | |
| ATOM | 869 | N | VAL | A1114 | 10.906 | 33.831 | 23.245 | 1.00 | 17.10 | N | |
| ATOM | 870 | CA | VAL | A1114 | 11.289 | 34.548 | 24.451 | 1.00 | 16.74 | C | |
| ATOM | 871 | CB | VAL | A1114 | 11.164 | 36.081 | 24.243 | 1.00 | 16.29 | C | |
| ATOM | 872 | CG1 | VAL | A1114 | 11.205 | 36.814 | 25.592 | 1.00 | 15.10 | C | |
| ATOM | 873 | CG2 | VAL | A1114 | 12.298 | 36.574 | 23.329 | 1.00 | 16.31 | C | |
| ATOM | 874 | C | VAL | A1114 | 10.422 | 34.149 | 25.635 | 1.00 | 17.36 | C | |
| ATOM | 875 | O | VAL | A1114 | 9.190 | 34.114 | 25.544 | 1.00 | 16.90 | O | |
| ATOM | 876 | N | ILE | A1115 | 11.073 | 33.834 | 26.747 | 1.00 | 17.78 | N | |
| ATOM | 877 | CA | ILE | A1115 | 10.354 | 33.478 | 27.957 | 1.00 | 18.59 | C | |
| ATOM | 878 | CB | ILE | A1115 | 10.632 | 32.020 | 28.395 | 1.00 | 17.89 | C | |
| ATOM | 879 | CG2 | ILE | A1115 | 9.806 | 31.691 | 29.641 | 1.00 | 17.73 | C | |
| ATOM | 880 | CG1 | ILE | A1115 | 10.247 | 31.053 | 27.272 | 1.00 | 18.13 | C | |
| ATOM | 881 | CD1 | ILE | A1115 | 10.654 | 29.599 | 27.521 | 1.00 | 17.22 | C | |
| ATOM | 882 | C | ILE | A1115 | 10.830 | 34.431 | 29.049 | 1.00 | 19.32 | C | |
| ATOM | 883 | O | ILE | A1115 | 12.025 | 34.538 | 29.304 | 1.00 | 19.25 | O | |
| ATOM | 884 | N | LEU | A1116 | 9.895 | 35.137 | 29.670 | 1.00 | 20.62 | N | |
| ATOM | 885 | CA | LEU | A1116 | 10.239 | 36.066 | 30.736 | 1.00 | 22.56 | C | |
| ATOM | 886 | CB | LEU | A1116 | 9.408 | 37.341 | 30.617 | 1.00 | 22.06 | C | |
| ATOM | 887 | CG | LEU | A1116 | 9.547 | 38.064 | 29.272 | 1.00 | 22.11 | C | |
| ATOM | 888 | CD1 | LEU | A1116 | 8.540 | 39.207 | 29.187 | 1.00 | 21.22 | C | |
| ATOM | 889 | CD2 | LEU | A1116 | 10.969 | 38.578 | 29.113 | 1.00 | 21.72 | C | |
| ATOM | 890 | C | LEU | A1116 | 9.984 | 35.379 | 32.069 | 1.00 | 24.06 | C | |
| ATOM | 891 | O | LEU | A1116 | 8.936 | 34.765 | 32.275 | 1.00 | 23.78 | O | |
| ATOM | 892 | N | GLU | A1117 | 10.953 | 35.486 | 32.970 | 1.00 | 25.93 | N | |
| ATOM | 893 | CA | GLU | A1117 | 10.857 | 34.843 | 34.267 | 1.00 | 28.06 | C | |
| ATOM | 894 | CB | GLU | A1117 | 11.604 | 33.502 | 34.204 | 1.00 | 28.05 | C | |
| ATOM | 895 | CG | GLU | A1117 | 11.800 | 32.793 | 35.536 | 1.00 | 28.32 | C | |
| ATOM | 896 | CD | GLU | A1117 | 12.626 | 31.512 | 35.405 | 1.00 | 28.66 | C | |
| ATOM | 897 | OE1 | GLU | A1117 | 13.711 | 31.567 | 34.777 | 1.00 | 28.33 | O | |
| ATOM | 898 | OE2 | GLU | A1117 | 12.196 | 30.458 | 35.936 | 1.00 | 28.07 | O | |
| ATOM | 899 | C | GLU | A1117 | 11.413 | 35.699 | 35.404 | 1.00 | 29.70 | C | |
| ATOM | 900 | O | GLU | A1117 | 12.242 | 36.583 | 35.190 | 1.00 | 29.81 | O | |
| ATOM | 901 | N | GLU | A1118 | 10.928 | 35.441 | 36.615 | 1.00 | 31.64 | N | |
| ATOM | 902 | CA | GLU | A1118 | 11.416 | 36.141 | 37.794 | 1.00 | 33.73 | C | |
| ATOM | 903 | CB | GLU | A1118 | 10.860 | 37.567 | 37.862 | 1.00 | 35.16 | C | |
| ATOM | 904 | CG | GLU | A1118 | 9.358 | 37.670 | 37.941 | 1.00 | 37.05 | C | |
| ATOM | 905 | CD | GLU | A1118 | 8.900 | 39.113 | 38.056 | 1.00 | 38.65 | C | |
| ATOM | 906 | OE1 | GLU | A1118 | 9.476 | 39.973 | 37.348 | 1.00 | 38.82 | O | |
| ATOM | 907 | OE2 | GLU | A1118 | 7.961 | 39.385 | 38.848 | 1.00 | 39.77 | O | |
| ATOM | 908 | C | GLU | A1118 | 11.043 | 35.365 | 39.048 | 1.00 | 34.16 | C | |
| ATOM | 909 | O | GLU | A1118 | 9.912 | 34.845 | 39.107 | 1.00 | 34.38 | O | |
| ATOM | 910 | OXT | GLU | A1118 | 11.894 | 35.280 | 39.956 | 1.00 | 35.21 | O | |
| TER | 911 | | GLU | A1118 | | | | | | | |
| ATOM | 912 | CB | MET | B2003 | 5.244 | 24.216 | 39.082 | 1.00 | 30.13 | C | |
| ATOM | 913 | CG | MET | B2003 | 5.229 | 22.763 | 38.655 | 1.00 | 32.03 | C | |
| ATOM | 914 | SD | MET | B2003 | 4.499 | 21.640 | 39.877 | 1.00 | 34.60 | S | |
| ATOM | 915 | CE | MET | B2003 | 5.729 | 20.323 | 39.875 | 1.00 | 32.91 | C | |
| ATOM | 916 | C | MET | B2003 | 5.364 | 24.946 | 36.697 | 1.00 | 27.68 | C | |
| ATOM | 917 | O | MET | B2003 | 4.190 | 25.254 | 36.454 | 1.00 | 27.76 | O | |
| ATOM | 918 | N | MET | B2003 | 5.824 | 26.555 | 38.533 | 1.00 | 28.66 | N | |
| ATOM | 919 | CA | MET | B2003 | 5.954 | 25.143 | 38.090 | 1.00 | 28.51 | C | |
| ATOM | 920 | N | ILE | B2004 | 6.184 | 24.439 | 35.782 | 1.00 | 26.12 | N | |
| ATOM | 921 | CA | ILE | B2004 | 5.730 | 24.192 | 34.423 | 1.00 | 24.36 | C | |
| ATOM | 922 | CB | ILE | B2004 | 6.907 | 24.200 | 33.423 | 1.00 | 23.97 | C | |
| ATOM | 923 | CG2 | ILE | B2004 | 6.445 | 23.691 | 32.062 | 1.00 | 22.87 | C | |
| ATOM | 924 | CG1 | ILE | B2004 | 7.465 | 25.624 | 33.300 | 1.00 | 23.67 | C | |
| ATOM | 925 | CD1 | ILE | B2004 | 8.670 | 25.746 | 32.379 | 1.00 | 23.02 | C | |
| ATOM | 926 | C | ILE | B2004 | 5.035 | 22.841 | 34.376 | 1.00 | 23.70 | C | |
| ATOM | 927 | O | ILE | B2004 | 5.537 | 21.854 | 34.917 | 1.00 | 23.27 | O | |
| ATOM | 928 | N | VAL | B2005 | 3.866 | 22.793 | 33.748 | 1.00 | 22.71 | N | |
| ATOM | 929 | CA | VAL | B2005 | 3.151 | 21.530 | 33.660 | 1.00 | 22.27 | C | |
| ATOM | 930 | CB | VAL | B2005 | 1.853 | 21.564 | 34.494 | 1.00 | 21.83 | C | |
| ATOM | 931 | CG1 | VAL | B2005 | 2.204 | 21.793 | 35.959 | 1.00 | 21.47 | C | |
| ATOM | 932 | CG2 | VAL | B2005 | 0.920 | 22.653 | 33.994 | 1.00 | 21.79 | C | |
| ATOM | 933 | C | VAL | B2005 | 2.847 | 21.133 | 32.228 | 1.00 | 22.00 | C | |
| ATOM | 934 | O | VAL | B2005 | 2.130 | 20.163 | 31.995 | 1.00 | 22.54 | O | |
| ATOM | 935 | N | GLY | B2006 | 3.417 | 21.880 | 31.278 | 1.00 | 21.53 | N | |
| ATOM | 936 | CA | GLY | B2006 | 3.225 | 21.594 | 29.867 | 1.00 | 20.47 | C | |
| ATOM | 937 | C | GLY | B2006 | 3.802 | 22.634 | 28.914 | 1.00 | 19.98 | C | |
| ATOM | 938 | O | GLY | B2006 | 3.873 | 23.820 | 29.237 | 1.00 | 20.12 | O | |
| ATOM | 939 | N | HIS | B2007 | 4.235 | 22.175 | 27.741 | 1.00 | 19.33 | N | |
| ATOM | 940 | CA | HIS | B2007 | 4.789 | 23.031 | 26.686 | 1.00 | 18.80 | C | |
| ATOM | 941 | CB | HIS | B2007 | 6.301 | 23.191 | 26.832 | 1.00 | 18.37 | C | |
| ATOM | 942 | CG | HIS | B2007 | 6.945 | 23.945 | 25.708 | 1.00 | 18.59 | C | |
| ATOM | 943 | CD2 | HIS | B2007 | 8.088 | 23.712 | 25.015 | 1.00 | 19.30 | C | |
| ATOM | 944 | ND1 | HIS | B2007 | 6.447 | 25.138 | 25.226 | 1.00 | 18.65 | N | |
| ATOM | 945 | CE1 | HIS | B2007 | 7.254 | 25.607 | 24.289 | 1.00 | 18.01 | C | |
| ATOM | 946 | NE2 | HIS | B2007 | 8.259 | 24.763 | 24.143 | 1.00 | 18.26 | N | |
| ATOM | 947 | C | HIS | B2007 | 4.482 | 22.387 | 25.342 | 1.00 | 18.69 | C | |
| ATOM | 948 | O | HIS | B2007 | 4.731 | 21.197 | 25.146 | 1.00 | 17.82 | O | |
| ATOM | 949 | N | GLY | B2008 | 3.925 | 23.174 | 24.425 | 1.00 | 19.16 | N | |
| ATOM | 950 | CA | GLY | B2008 | 3.599 | 22.655 | 23.110 | 1.00 | 18.31 | C | |
| ATOM | 951 | C | GLY | B2008 | 3.703 | 23.724 | 22.043 | 1.00 | 18.66 | C | |
| ATOM | 952 | O | GLY | B2008 | 3.378 | 24.890 | 22.297 | 1.00 | 18.62 | O | |
| ATOM | 953 | N | ILE | B2009 | 4.175 | 23.336 | 20.858 | 1.00 | 18.12 | N | |
| ATOM | 954 | CA | ILE | B2009 | 4.282 | 24.257 | 19.736 | 1.00 | 18.56 | C | |
| ATOM | 955 | CB | ILE | B2009 | 5.747 | 24.562 | 19.351 | 1.00 | 18.23 | C | |
| ATOM | 956 | CG2 | ILE | B2009 | 6.471 | 25.174 | 20.538 | 1.00 | 17.94 | C | |
| ATOM | 957 | CG1 | ILE | B2009 | 6.440 | 23.285 | 18.847 | 1.00 | 17.91 | C | |
| ATOM | 958 | CD1 | ILE | B2009 | 7.819 | 23.525 | 18.220 | 1.00 | 16.62 | C | |
| ATOM | 959 | C | ILE | B2009 | 3.601 | 23.657 | 18.513 | 1.00 | 19.50 | C | |
| ATOM | 960 | O | ILE | B2009 | 3.286 | 22.470 | 18.472 | 1.00 | 18.95 | O | |
| ATOM | 961 | N | ASP | B2010 | 3.356 | 24.492 | 17.518 | 1.00 | 20.39 | N | |
| ATOM | 962 | CA | ASP | B2010 | 2.751 | 24.015 | 16.298 | 1.00 | 21.57 | C | |
| ATOM | 963 | CB | ASP | B2010 | 1.251 | 23.773 | 16.481 | 1.00 | 22.40 | C | |
| ATOM | 964 | CG | ASP | B2010 | 0.635 | 22.980 | 15.317 | 1.00 | 23.74 | C | |
| ATOM | 965 | OD1 | ASP | B2010 | 0.223 | 23.595 | 14.308 | 1.00 | 23.88 | O | |
| ATOM | 966 | OD2 | ASP | B2010 | 0.576 | 21.731 | 15.415 | 1.00 | 24.74 | O | |
| ATOM | 967 | C | ASP | B2010 | 2.979 | 25.019 | 15.200 | 1.00 | 22.11 | C | |
| ATOM | 968 | O | ASP | B2010 | 3.032 | 26.224 | 15.443 | 1.00 | 22.03 | O | |
| ATOM | 969 | N | ILE | B2011 | 3.145 | 24.502 | 13.991 | 1.00 | 23.00 | N | |
| ATOM | 970 | CA | ILE | B2011 | 3.335 | 25.336 | 12.824 | 1.00 | 24.24 | C | |
| ATOM | 971 | CB | ILE | B2011 | 4.818 | 25.320 | 12.339 | 1.00 | 24.43 | C | |
| ATOM | 972 | CG2 | ILE | B2011 | 5.258 | 23.905 | 11.972 | 1.00 | 24.62 | C | |
| ATOM | 973 | CG1 | ILE | B2011 | 4.970 | 26.244 | 11.133 | 1.00 | 25.18 | C | |
| ATOM | 974 | CD1 | ILE | B2011 | 6.404 | 26.433 | 10.684 | 1.00 | 26.49 | C | |
| ATOM | 975 | C | ILE | B2011 | 2.398 | 24.818 | 11.732 | 1.00 | 25.03 | C | |
| ATOM | 976 | O | ILE | B2011 | 2.257 | 23.609 | 11.532 | 1.00 | 24.85 | O | |
| ATOM | 977 | N | GLU | B2011 | 1.733 | 25.740 | 11.050 | 1.00 | 26.14 | N | |
| ATOM | 978 | CA | GLU | B2011 | 0.813 | 25.377 | 9.979 | 1.00 | 27.22 | C | |
| ATOM | 979 | CB | GLU | B2011 | 0.636 | 25.692 | 10.369 | 1.00 | 28.06 | C | |
| ATOM | 980 | CG | GLU | B2012 | 1.179 | 24.920 | 11.549 | 1.00 | 30.35 | C | |
| ATOM | 981 | CD | GLU | B2011 | 1.237 | 23.427 | 11.301 | 1.00 | 31.85 | C | |
| ATOM | 982 | OE1 | GLU | B2011 | 1.472 | 23.026 | 10.133 | 1.00 | 32.85 | O | |
| ATOM | 983 | OE2 | GLU | B2011 | 1.065 | 22.660 | 12.278 | 1.00 | 31.85 | O | |
| ATOM | 984 | C | GLU | B2011 | 1.130 | 26.138 | 8.704 | 1.00 | 27.07 | C | |
| ATOM | 985 | O | GLU | B2012 | 1.558 | 27.294 | 8.738 | 1.00 | 26.78 | O | |
| ATOM | 986 | N | GLU | B2013 | 0.914 | 25.471 | 7.579 | 1.00 | 27.58 | N | |
| ATOM | 987 | CA | GLU | B2013 | 1.115 | 26.080 | 6.272 | 1.00 | 27.80 | C | |
| ATOM | 988 | CB | GLU | B2013 | 1.533 | 25.018 | 5.253 | 1.00 | 28.06 | C | |
| ATOM | 989 | CG | GLU | B2013 | 1.806 | 25.553 | 3.851 | 1.00 | 29.06 | C | |
| ATOM | 990 | CD | GLU | B2013 | 2.105 | 24.447 | 2.858 | 0.05 | 28.78 | C | |
| ATOM | 991 | OE1 | GLU | B2013 | 3.074 | 23.691 | 3.081 | 0.05 | 28.87 | O | |
| ATOM | 992 | OE2 | GLU | B2013 | 1.370 | 24.335 | 1.856 | 0.05 | 28.90 | O | |
| ATOM | 993 | C | GLU | B2013 | −0.268 | 26.626 | 5.939 | 1.00 | 27.67 | C | |
| ATOM | 994 | O | GLU | B2013 | −1.250 | 25.894 | 5.982 | 1.00 | 27.29 | O | |
| ATOM | 995 | N | LEU | B2014 | −0.350 | 27.916 | 5.644 | 1.00 | 28.07 | N | |
| ATOM | 996 | CA | LEU | B2014 | −1.629 | 28.542 | 5.330 | 1.00 | 28.65 | C | |
| ATOM | 997 | CB | LEU | B2014 | −1.426 | 30.023 | 4.986 | 1.00 | 28.56 | C | |
| ATOM | 998 | CG | LEU | B2014 | −1.332 | 31.022 | 6.147 | 1.00 | 28.97 | C | |
| ATOM | 999 | CD1 | LEU | B2014 | −2.677 | 31.113 | 6.840 | 1.00 | 28.76 | C | |
| ATOM | 1000 | CD2 | LEU | B2014 | −0.260 | 30.593 | 7.139 | 1.00 | 28.89 | C | |
| ATOM | 1001 | C | LEU | B2014 | −2.356 | 27.847 | 4.189 | 1.00 | 28.89 | C | |
| ATOM | 1002 | O | LEU | B2014 | −3.584 | 27.758 | 4.189 | 1.00 | 28.92 | O | |
| ATOM | 1003 | N | ALA | B2015 | −1.588 | 27.346 | 3.228 | 1.00 | 29.40 | N | |
| ATOM | 1004 | CA | ALA | B2015 | −2.137 | 26.665 | 2.058 | 1.00 | 29.93 | C | |
| ATOM | 1005 | CB | ALA | B2015 | −0.999 | 26.206 | 1.146 | 1.00 | 29.23 | C | |
| ATOM | 1006 | C | ALA | B2015 | −3.033 | 25.483 | 2.401 | 1.00 | 30.15 | C | |
| ATOM | 1007 | O | ALA | B2015 | −4.062 | 25.278 | 1.760 | 1.00 | 30.32 | O | |
| ATOM | 1008 | N | SER | D2016 | −2.648 | 24.703 | 3.403 | 1.00 | 30.53 | N | |
| ATOM | 1009 | CA | SER | B2016 | −3.445 | 23.544 | 3.776 | 1.00 | 31.47 | C | |
| ATOM | 1010 | CB | SER | B2016 | −2.625 | 22.583 | 4.640 | 1.00 | 31.89 | C | |
| ATOM | 1011 | OG | SER | B2016 | −2.229 | 23.193 | 5.846 | 1.00 | 33.02 | O | |
| ATOM | 1012 | C | SER | B2016 | −4.735 | 23.946 | 4.487 | 1.00 | 31.84 | C | |
| ATOM | 1013 | O | SER | B2016 | −5.741 | 23.243 | 4.391 | 1.00 | 31.71 | O | |
| ATOM | 1014 | N | ILE | B2017 | −4.716 | 25.068 | 5.201 | 1.00 | 32.07 | N | |
| ATOM | 1015 | CA | ILE | B2017 | −5.932 | 25.527 | 5.861 | 1.00 | 32.52 | C | |
| ATOM | 1016 | CB | ILE | B2017 | −5.651 | 26.688 | 6.843 | 1.00 | 32.37 | C | |
| ATOM | 1017 | CG2 | ILE | B2017 | −6.973 | 27.246 | 7.385 | 1.00 | 32.53 | C | |
| ATOM | 1018 | CG1 | ILE | B2017 | −4.759 | 26.203 | 7.996 | 1.00 | 32.12 | C | |
| ATOM | 1019 | CD1 | ILE | B2017 | −5.399 | 25.165 | 8.894 | 1.00 | 31.06 | C | |
| ATOM | 1020 | C | ILE | B2017 | −6.862 | 26.015 | 4.748 | 1.00 | 33.04 | C | |
| ATOM | 1021 | O | ILE | B2017 | −8.084 | 25.862 | 4.821 | 1.00 | 32.69 | O | |
| ATOM | 1022 | N | GLU | B2018 | −6.263 | 26.597 | 3.711 | 1.00 | 33.86 | N | |
| ATOM | 1023 | CA | GLU | B2018 | −7.006 | 27.095 | 2.554 | 1.00 | 34.86 | C | |
| ATOM | 1024 | CB | GLU | B2018 | −6.042 | 27.669 | 1.504 | 1.00 | 36.31 | C | |
| ATOM | 1025 | CG | GLU | B2018 | −5.544 | 29.080 | 1.764 | 1.00 | 38.14 | C | |
| ATOM | 1026 | CD | GLU | B2018 | −6.554 | 30.137 | 1.359 | 1.00 | 39.70 | C | |
| ATOM | 1027 | OE1 | GLU | B2018 | −7.673 | 29.764 | 0.937 | 1.00 | 40.41 | O | |
| ATOM | 1028 | OE2 | GLU | B2018 | −6.231 | 31.341 | 1.464 | 1.00 | 40.25 | O | |
| ATOM | 1029 | C | GLU | B2018 | −7.782 | 25.947 | 1.918 | 1.00 | 34.57 | C | |
| ATOM | 1030 | O | GLU | B2018 | −8.964 | 26.076 | 1.612 | 1.00 | 34.08 | O | |
| ATOM | 1031 | N | SER | B2019 | −7.094 | 24.828 | 1.711 | 1.00 | 34.49 | N | |
| ATOM | 1032 | CA | SER | B2019 | −7.705 | 23.650 | 1.103 | 1.00 | 34.60 | C | |
| ATOM | 1033 | CB | SER | B2019 | −6.639 | 22.610 | 0.793 | 1.00 | 34.46 | C | |
| ATOM | 1034 | OG | SER | B2019 | −5.712 | 23.150 | −0.125 | 1.00 | 35.85 | O | |
| ATOM | 1035 | C | SER | B2019 | −8.761 | 23.039 | 1.996 | 1.00 | 34.54 | C | |
| ATOM | 1036 | O | SER | B2019 | −9.827 | 22.646 | 1.523 | 1.00 | 34.67 | O | |
| ATOM | 1037 | N | ALA | B2020 | −8.463 | 22.965 | 3.290 | 1.00 | 34.29 | N | |
| ATOM | 1038 | CA | ALA | B2020 | −9.395 | 22.402 | 4.251 | 1.00 | 34.25 | C | |
| ATOM | 1039 | CB | ALA | B2020 | −8.811 | 22.471 | 5.650 | 1.00 | 33.91 | C | |
| ATOM | 1040 | C | ALA | B2020 | −10.703 | 23.175 | 4.180 | 1.00 | 34.65 | C | |
| ATOM | 1041 | O | ALA | B2020 | −11.778 | 22.579 | 4.133 | 1.00 | 34.60 | O | |
| ATOM | 1042 | N | VAL | B2021 | −10.604 | 24.502 | 4.151 | 1.00 | 35.08 | N | |
| ATOM | 1043 | CA | VAL | B2021 | −11.784 | 25.359 | 4.073 | 1.00 | 35.39 | C | |
| ATOM | 1044 | CB | VAL | B2021 | −11.411 | 26.855 | 4.220 | 1.00 | 35.27 | C | |
| ATOM | 1045 | CG1 | VAL | B2021 | −12.608 | 27.732 | 3.884 | 1.00 | 34.79 | C | |
| ATOM | 1046 | CG2 | VAL | B2021 | −10.951 | 27.136 | 5.637 | 1.00 | 35.33 | C | |
| ATOM | 1047 | C | VAL | B2021 | −12.526 | 25.174 | 2.756 | 1.00 | 36.00 | C | |
| ATOM | 1048 | O | VAL | B2021 | −13.746 | 24.985 | 2.749 | 1.00 | 35.93 | O | |
| ATOM | 1049 | N | THR | B2022 | −11.795 | 25.237 | 1.644 | 1.00 | 36.68 | N | |
| ATOM | 1050 | CA | THR | B2022 | −12.401 | 25.072 | 0.326 | 1.00 | 37.80 | C | |
| ATOM | 1051 | CB | THR | B2022 | −11.351 | 25.211 | −0.813 | 1.00 | 38.05 | C | |
| ATOM | 1052 | OG1 | THR | B2022 | −10.830 | 26.549 | −0.839 | 1.00 | 37.71 | O | |
| ATOM | 1053 | CG2 | THR | B2022 | −11.988 | 24.898 | −2.166 | 1.00 | 37.87 | C | |
| ATOM | 1054 | C | THR | B2022 | −13.064 | 23.701 | 0.210 | 1.00 | 38.58 | C | |
| ATOM | 1055 | O | THR | B2022 | −14.204 | 23.584 | −0.234 | 1.00 | 38.63 | O | |
| ATOM | 1056 | N | ARG | B2023 | −12.348 | 22.666 | 0.629 | 1.00 | 39.65 | N | |
| ATOM | 1057 | CA | ARG | B2023 | −12.864 | 21.306 | 0.563 | 1.00 | 41.03 | C | |
| ATOM | 1058 | CB | ARG | B2023 | −11.828 | 20.317 | 1.106 | 1.00 | 41.66 | C | |
| ATOM | 1059 | CG | ARG | B2023 | −11.891 | 18.939 | 0.452 | 1.00 | 43.04 | C | |
| ATOM | 1060 | CD | ARG | B2023 | −13.312 | 18.416 | 0.395 | 1.00 | 43.90 | C | |
| ATOM | 1061 | NE | ARG | B2023 | −13.406 | 17.090 | −0.202 | 1.00 | 44.75 | N | |
| ATOM | 1062 | CZ | ARG | B2023 | −14.524 | 16.371 | −0.231 | 1.00 | 45.34 | C | |
| ATOM | 1063 | NH1 | ARG | B2023 | −15.638 | 16.862 | 0.302 | 1.00 | 45.03 | N | |
| ATOM | 1064 | NH2 | ARG | B2023 | −14.527 | 15.157 | −0.773 | 1.00 | 45.61 | N | |
| ATOM | 1065 | C | ARG | B2023 | −14.165 | 21.138 | 1.343 | 1.00 | 41.52 | C | |
| ATOM | 1066 | O | ARG | B2023 | −15.180 | 20.712 | 0.787 | 1.00 | 41.49 | O | |
| ATOM | 1067 | N | HIS | B2024 | −14.120 | 21.467 | 2.631 | 1.00 | 41.94 | N | |
| ATOM | 1068 | CA | HIS | B2024 | −15.274 | 21.343 | 3.511 | 1.00 | 42.45 | C | |
| ATOM | 1069 | CB | HIS | B2024 | −14.931 | 20.424 | 4.678 | 1.00 | 43.39 | C | |
| ATOM | 1070 | CG | HIS | B2024 | −14.516 | 19.050 | 4.255 | 1.00 | 44.50 | C | |
| ATOM | 1071 | CD2 | HIS | B2024 | −13.294 | 18.468 | 4.191 | 1.00 | 44.72 | C | |
| ATOM | 1072 | ND1 | HIS | B2024 | −15.411 | 18.115 | 3.777 | 1.00 | 45.05 | N | |
| ATOM | 1073 | CE1 | HIS | B2024 | −14.758 | 17.018 | 3.436 | 1.00 | 45.22 | C | |
| ATOM | 1074 | NE2 | HIS | B2024 | −13.473 | 17.206 | 3.677 | 1.00 | 45.24 | N | |
| ATOM | 1075 | C | HIS | B2024 | −15.713 | 22.697 | 4.040 | 1.00 | 42.58 | C | |
| ATOM | 1076 | O | HIS | B2024 | −14.934 | 23.412 | 4.672 | 1.00 | 42.87 | O | |
| ATOM | 1077 | N | GLU | B2025 | −16.967 | 23.050 | 3.781 | 1.00 | 42.39 | N | |
| ATOM | 1078 | CA | GLU | B2025 | −17.490 | 24.326 | 4.242 | 1.00 | 41.91 | C | |
| ATOM | 1079 | CB | GLU | B2025 | −18.732 | 24.732 | 3.437 | 1.00 | 43.59 | C | |
| ATOM | 1080 | CG | GLU | B2025 | −18.797 | 26.229 | 3.153 | 1.00 | 45.80 | C | |
| ATOM | 1081 | CD | GLU | B2025 | −20.117 | 26.675 | 2.544 | 1.00 | 47.26 | C | |
| ATOM | 1082 | OE1 | GLU | B2025 | −20.610 | 26.003 | 1.603 | 1.00 | 47.52 | O | |
| ATOM | 1083 | OE2 | GLU | B2025 | −20.653 | 27.710 | 3.006 | 1.00 | 48.07 | O | |
| ATOM | 1084 | C | GLU | B2025 | −17.848 | 24.211 | 5.712 | 1.00 | 40.39 | C | |
| ATOM | 1085 | O | GLU | B2025 | −18.172 | 25.203 | 6.356 | 1.00 | 40.62 | O | |
| ATOM | 1086 | N | GLY | B2026 | −17.789 | 22.993 | 6.238 | 1.00 | 38.87 | N | |
| ATOM | 1087 | CA | GLY | B2026 | −18.105 | 22.788 | 7.638 | 1.00 | 36.84 | C | |
| ATOM | 1088 | C | GLY | B2026 | −16.865 | 22.836 | 8.507 | 1.00 | 35.50 | C | |
| ATOM | 1089 | O | GLY | B2026 | −16.954 | 22.961 | 9.732 | 1.00 | 35.89 | O | |
| ATOM | 1090 | N | PHE | B2027 | −15.706 | 22.746 | 7.866 | 1.00 | 33.86 | N | |
| ATOM | 1091 | CA | PHE | B2027 | −14.416 | 22.752 | 8.549 | 1.00 | 32.08 | C | |
| ATOM | 1092 | CB | PHE | B2027 | −13.293 | 22.958 | 7.532 | 1.00 | 31.28 | C | |
| ATOM | 1093 | CG | PHE | B2027 | −11.926 | 22.896 | 8.128 | 1.00 | 29.92 | C | |
| ATOM | 1094 | CD1 | PHE | B2027 | −11.396 | 21.682 | 8.555 | 1.00 | 28.97 | C | |
| ATOM | 1095 | CD2 | PHE | B2027 | −11.176 | 24.054 | 8.293 | 1.00 | 29.17 | C | |
| ATOM | 1096 | CE1 | PHE | B2027 | −10.143 | 21.623 | 9.135 | 1.00 | 28.66 | C | |
| ATOM | 1097 | CE2 | PHE | B2027 | −9.918 | 24.006 | 8.875 | 1.00 | 28.77 | C | |
| ATOM | 1098 | CZ | PHE | B2027 | −9.399 | 22.790 | 9.298 | 1.00 | 28.68 | C | |
| ATOM | 1099 | C | PHE | B2027 | −14.294 | 23.801 | 9.651 | 1.00 | 31.46 | C | |
| ATOM | 1100 | O | PHE | B2027 | −14.059 | 23.465 | 10.815 | 1.00 | 31.23 | O | |
| ATOM | 1101 | N | ALA | B2028 | −14.454 | 25.067 | 9.278 | 1.00 | 30.51 | N | |
| ATOM | 1102 | CA | ALA | B2028 | −14.360 | 26.169 | 10.228 | 1.00 | 30.25 | C | |
| ATOM | 1103 | CB | ALA | B2028 | −14.624 | 27.487 | 9.517 | 1.00 | 30.53 | C | |
| ATOM | 1104 | C | ALA | B2028 | −15.305 | 26.030 | 11.421 | 1.00 | 30.13 | C | |
| ATOM | 1105 | O | ALA | B2028 | −14.926 | 26.308 | 12.561 | 1.00 | 29.80 | O | |
| ATOM | 1106 | N | LYS | B2029 | −16.533 | 25.600 | 11.160 | 1.00 | 30.02 | N | |
| ATOM | 1107 | CA | LYS | B2029 | −17.520 | 25.448 | 12.225 | 1.00 | 30.10 | C | |
| ATOM | 1108 | CB | LYS | B2029 | −18.882 | 25.053 | 11.637 | 1.00 | 30.49 | C | |
| ATOM | 1109 | CG | LYS | B2029 | −19.552 | 26.148 | 10.821 | 1.00 | 31.20 | C | |
| ATOM | 1110 | CD | LYS | B2029 | −20.765 | 25.618 | 10.074 | 0.05 | 30.87 | C | |
| ATOM | 1111 | CE | LYS | B2029 | −21.354 | 26.675 | 9.153 | 0.05 | 30.89 | C | |
| ATOM | 1112 | NZ | LYS | B2029 | −22.504 | 26.150 | 8.365 | 0.05 | 30.76 | N | |
| ATOM | 1113 | C | LYS | B2029 | −17.099 | 24.408 | 13.248 | 1.00 | 29.50 | C | |
| ATOM | 1114 | O | LYS | B2029 | −17.467 | 24.491 | 14.420 | 1.00 | 29.99 | O | |
| ATOM | 1115 | N | ARG | B2030 | −16.326 | 23.428 | 12.798 | 1.00 | 28.70 | N | |
| ATOM | 1116 | CA | ARG | B2030 | −15.877 | 22.348 | 13.667 | 1.00 | 27.83 | C | |
| ATOM | 1117 | CB | ARG | B2030 | −15.616 | 21.093 | 12.822 | 1.00 | 27.88 | C | |
| ATOM | 1118 | CG | ARG | B2030 | −16.867 | 20.503 | 12.193 | 0.05 | 27.87 | C | |
| ATOM | 1119 | CD | ARG | B2030 | −16.543 | 19.293 | 11.332 | 0.05 | 27.87 | C | |
| ATOM | 1120 | NE | ARG | B2030 | −15.748 | 19.648 | 10.160 | 0.05 | 27.89 | N | |
| ATOM | 1121 | CZ | ARG | B2030 | −15.379 | 18.785 | 9.218 | 0.05 | 27.88 | C | |
| ATOM | 1122 | NH1 | ARG | B2030 | −15.732 | 17.510 | 9.309 | 0.05 | 27.89 | N | |
| ATOM | 1123 | NH2 | ARG | B2030 | −14.658 | 19.196 | 8.184 | 0.05 | 27.87 | N | |
| ATOM | 1124 | C | ARG | B2030 | −14.631 | 22.698 | 14.477 | 1.00 | 26.83 | C | |
| ATOM | 1125 | O | ARG | B2030 | −14.391 | 22.134 | 15.541 | 1.00 | 26.57 | O | |
| ATOM | 1126 | N | VAL | B2031 | −13.854 | 23.644 | 13.965 | 1.00 | 25.79 | N | |
| ATOM | 1127 | CA | VAL | B2031 | −12.610 | 24.073 | 14.596 | 1.00 | 24.76 | C | |
| ATOM | 1128 | CB | VAL | B2031 | −11.565 | 24.442 | 13.505 | 1.00 | 24.82 | C | |
| ATOM | 1129 | CG1 | VAL | B2031 | −10.365 | 25.139 | 14.121 | 1.00 | 24.72 | C | |
| ATOM | 1130 | CG2 | VAL | B2031 | −11.125 | 23.184 | 12.779 | 1.00 | 24.93 | C | |
| ATOM | 1131 | C | VAL | B2031 | −12.806 | 25.263 | 15.523 | 1.00 | 23.81 | C | |
| ATOM | 1132 | O | VAL | B2031 | −12.166 | 25.365 | 16.568 | 1.00 | 22.82 | O | |
| ATOM | 1133 | N | LEU | B2032 | −13.709 | 26.149 | 15.127 | 1.00 | 23.54 | N | |
| ATOM | 1134 | CA | LEU | B2032 | −13.997 | 27.361 | 15.873 | 1.00 | 23.22 | C | |
| ATOM | 1135 | CB | LEU | B2032 | −14.157 | 28.528 | 14.893 | 1.00 | 22.85 | C | |
| ATOM | 1136 | CG | LEU | B2032 | −12.924 | 28.789 | 14.022 | 1.00 | 22.24 | C | |
| ATOM | 1137 | CD1 | LEU | B2032 | −13.210 | 29.906 | 13.026 | 1.00 | 22.1.1 | C | |
| ATOM | 1138 | CD2 | LEU | B2032 | −11.747 | 29.152 | 14.918 | 1.00 | 22.17 | C | |
| ATOM | 1139 | C | LEU | B2032 | −15.233 | 27.256 | 16.744 | 1.00 | 23.32 | C | |
| ATOM | 1140 | O | LEU | B2032 | −16.122 | 26.454 | 16.482 | 1.00 | 23.42 | O | |
| ATOM | 1141 | N | THR | B2033 | −15.263 | 28.060 | 17.803 | 1.00 | 23.69 | N | |
| ATOM | 1142 | CA | THR | B2033 | −16.404 | 28.105 | 18.713 | 1.00 | 23.81 | C | |
| ATOM | 1143 | CB | THR | B2033 | −16.002 | 28.538 | 20.129 | 1.00 | 23.49 | C | |
| ATOM | 1144 | OG1 | THR | B2033 | −15.442 | 29.858 | 20.074 | 1.00 | 23.22 | O | |
| ATOM | 1145 | CG2 | THR | B2033 | −15.004 | 27.571 | 20.726 | 1.00 | 22.82 | C | |
| ATOM | 1146 | C | THR | B2033 | −17.312 | 29.191 | 18.162 | 1.00 | 24.17 | C | |
| ATOM | 1147 | O | THR | B2033 | −16.915 | 29.930 | 17.264 | 1.00 | 24.05 | O | |
| ATOM | 1148 | N | ALA | B2034 | −18.516 | 29.302 | 18.709 | 1.00 | 24.73 | N | |
| ATOM | 1149 | CA | ALA | B2034 | −19.456 | 30.322 | 18.246 | 1.00 | 25.88 | C | |
| ATOM | 1150 | CB | ALA | B2034 | −20.704 | 30.326 | 19.127 | 1.00 | 25.52 | C | |
| ATOM | 1151 | C | ALA | B2034 | −18.793 | 31.704 | 18.245 | 1.00 | 26.34 | C | |
| ATOM | 1152 | O | ALA | B2034 | −18.815 | 32.409 | 17.231 | 1.00 | 26.66 | O | |
| ATOM | 1153 | N | LEU | B2035 | −18.194 | 32.083 | 19.374 | 1.00 | 27.02 | N | |
| ATOM | 1154 | CA | LEU | B2035 | −17.518 | 33.375 | 19.477 | 1.00 | 27.85 | C | |
| ATOM | 1155 | CB | LEU | B2035 | −16.868 | 33.549 | 20.852 | 1.00 | 28.57 | C | |
| ATOM | 1156 | CG | LEU | B2035 | −17.760 | 33.942 | 22.028 | 1.00 | 29.51 | C | |
| ATOM | 1157 | CD1 | LEU | B2035 | −16.879 | 34.241 | 23.246 | 1.00 | 29.47 | C | |
| ATOM | 1158 | CD2 | LEU | B2035 | −18.596 | 35.181 | 21.648 | 1.00 | 29.85 | C | |
| ATOM | 1159 | C | LEU | B2035 | −16.445 | 33.516 | 18.405 | 1.00 | 28.07 | C | |
| ATOM | 1160 | O | LEU | B2035 | −16.395 | 34.516 | 11.687 | 1.00 | 28.48 | O | |
| ATOM | 1161 | N | GLU | B2036 | −15.578 | 32.518 | 18.308 | 1.00 | 27.90 | N | |
| ATOM | 1162 | CA | GLU | B2036 | −14.518 | 32.542 | 17.310 | 1.00 | 27.73 | C | |
| ATOM | 1163 | CB | GLU | B2036 | −13.666 | 31.270 | 17.414 | 1.00 | 26.50 | C | |
| ATOM | 1164 | CG | GLU | B2036 | −12.568 | 31.339 | 18.474 | 1.00 | 25.25 | C | |
| ATOM | 1165 | CD | GLU | B2036 | −11.969 | 29.976 | 18.799 | 1.00 | 24.47 | C | |
| ATOM | 1166 | OE1 | GLU | B2036 | −10.797 | 29.925 | 19.226 | 1.00 | 23.46 | O | |
| ATOM | 1167 | OE2 | GLU | B2036 | −12.676 | 28.957 | 18.636 | 1.00 | 24.23 | O | |
| ATOM | 1166 | C | GLU | B2036 | −15.073 | 32.686 | 15.895 | 1.00 | 28.15 | C | |
| ATOM | 1169 | O | GLU | B2036 | −14.507 | 33.403 | 15.083 | 1.00 | 28.18 | O | |
| ATOM | 1170 | N | MET | B2037 | −16.169 | 31.990 | 15.604 | 1.00 | 29.30 | N | |
| ATOM | 1171 | CA | MET | B2037 | −16.800 | 32.038 | 14.283 | 1.00 | 30.55 | C | |
| ATOM | 1172 | CB | MET | B2037 | −17.984 | 31.069 | 14.211 | 1.00 | 30.95 | C | |
| ATOM | 1173 | CG | MET | B2037 | −17.607 | 29.662 | 13.809 | 1.00 | 31.93 | C | |
| ATOM | 1174 | SD | MET | B2037 | −16.851 | 29.650 | 12.173 | 1.00 | 33.36 | S | |
| ATOM | 1175 | CE | MET | B2037 | −18.331 | 29.638 | 11.166 | 1.00 | 32.65 | C | |
| ATOM | 1176 | C | MET | B2037 | −17.286 | 33.436 | 13.934 | 1.00 | 31.15 | C | |
| ATOM | 1177 | O | MET | B2037 | −17.206 | 33.854 | 12.785 | 1.00 | 30.76 | O | |
| ATOM | 1178 | N | GLU | B2038 | −17.789 | 34.153 | 14.934 | 1.00 | 32.42 | N | |
| ATOM | 1179 | CA | GLU | B2038 | −18.278 | 35.513 | 14.730 | 1.00 | 33.88 | C | |
| ATOM | 1180 | CB | GLU | B2038 | −18.841 | 36.062 | 16.044 | 1.00 | 35.07 | C | |
| ATOM | 1181 | CG | GLU | B2038 | −19.432 | 37.455 | 15.931 | 1.00 | 37.91 | C | |
| ATOM | 1182 | CD | GLU | B2038 | −20.528 | 37.714 | 16.955 | 1.00 | 39.72 | C | |
| ATOM | 1183 | OE1 | GLU | B2038 | −21.587 | 37.050 | 16.864 | 1.00 | 40.71 | O | |
| ATOM | 1184 | OE2 | GLU | B2038 | −20.333 | 38.576 | 17.846 | 1.00 | 40.42 | O | |
| ATOM | 1185 | C | GLU | B2038 | −17.163 | 36.418 | 14.198 | 1.00 | 34.07 | C | |
| ATOM | 1186 | O | GLU | B2038 | −17.406 | 37.320 | 13.397 | 1.00 | 33.98 | O | |
| ATOM | 1187 | N | ARG | B2039 | −15.936 | 36.181 | 14.649 | 1.00 | 34.44 | N | |
| ATOM | 1188 | CA | ARG | B2039 | −14.810 | 36.972 | 14.179 | 1.00 | 35.18 | C | |
| ATOM | 1189 | CB | ARG | B2039 | −13.630 | 36.856 | 15.153 | 1.00 | 35.30 | C | |
| ATOM | 1190 | CG | ARG | B2039 | −12.370 | 37.601 | 14.720 | 1.00 | 35.26 | C | |
| ATOM | 1191 | CD | ARG | B2039 | −12.590 | 39.099 | 14.608 | 0.05 | 35.23 | C | |
| ATOM | 1192 | NE | ARG | B2039 | −11.358 | 39.788 | 14.235 | 0.05 | 35.24 | N | |
| ATOM | 1193 | CZ | ARG | B2039 | −11.260 | 41.099 | 14.040 | 0.05 | 35.24 | C | |
| ATOM | 1194 | NH1 | ARG | B2039 | −10.095 | 41.634 | 13.703 | 0.05 | 35.27 | N | |
| ATOM | 1195 | NH2 | ARG | B2039 | −12.325 | 41.875 | 14.182 | 0.05 | 35.27 | N | |
| ATOM | 1196 | C | ARG | B2039 | −14.418 | 36.445 | 12.797 | 1.00 | 35.34 | C | |
| ATOM | 1197 | O | ARG | B2039 | −14.167 | 37.217 | 11.868 | 1.00 | 35.66 | O | |
| ATOM | 1198 | N | PHE | B2040 | −14.382 | 35.123 | 12.673 | 1.00 | 35.29 | N | |
| ATOM | 1199 | CA | PHE | B2040 | −14.042 | 34.473 | 11.414 | 1.00 | 35.77 | C | |
| ATOM | 1200 | CB | PHE | B2040 | −14.241 | 32.961 | 11.545 | 1.00 | 34.55 | C | |
| ATOM | 1201 | CG | PHE | B2040 | −13.996 | 32.201 | 10.271 | 1.00 | 33.98 | C | |
| ATOM | 1202 | CD1 | PHE | B2040 | −12.716 | 32.122 | 9.725 | 1.00 | 33.52 | C | |
| ATOM | 1203 | CD2 | PHE | B2040 | −15.045 | 31.564 | 9.614 | 1.00 | 33.68 | C | |
| ATOM | 1204 | CE1 | PHE | B2040 | −12.488 | 31.420 | 8.548 | 1.00 | 33.41 | C | |
| ATOM | 1205 | CE2 | PHE | B2040 | −14.828 | 30.861 | 8.435 | 1.00 | 33.27 | C | |
| ATOM | 1206 | CZ | PHE | B2040 | −13.549 | 30.787 | 7.899 | 1.00 | 33.46 | C | |
| ATOM | 1207 | C | PHE | B2040 | −14.916 | 34.997 | 10.268 | 1.00 | 36.68 | C | |
| ATOM | 1208 | O | PHE | B2040 | −14.445 | 35.199 | 9.144 | 1.00 | 36.38 | O | |
| ATOM | 1209 | N | THR | B2041 | −16.191 | 35.221 | 10.561 | 1.00 | 37.87 | N | |
| ATOM | 1210 | CA | THR | B2041 | −17.121 | 35.692 | 9.546 | 1.00 | 39.25 | C | |
| ATOM | 1211 | CB | THR | B2041 | −18.580 | 35.450 | 9.981 | 1.00 | 39.24 | C | |
| ATOM | 1212 | OG1 | THR | B2041 | −18.811 | 34.041 | 10.115 | 1.00 | 39.45 | O | |
| ATOM | 1213 | CG2 | THR | B2041 | −19.540 | 36.001 | 8.943 | 1.00 | 40.16 | C | |
| ATOM | 1214 | C | THR | B2041 | −16.947 | 37.159 | 9.159 | 1.00 | 39.72 | C | |
| ATOM | 1215 | O | THR | B2041 | −17.496 | 37.598 | 8.157 | 1.00 | 40.46 | O | |
| ATOM | 1216 | N | SER | B2042 | −16.175 | 37.915 | 9.930 | 1.00 | 40.14 | N | |
| ATOM | 1217 | CA | SER | B2042 | −15.970 | 39.327 | 9.609 | 1.00 | 40.54 | C | |
| ATOM | 1218 | CB | SER | B2042 | −16.159 | 40.184 | 10.8S8 | 1.00 | 40.81 | C | |
| ATOM | 1219 | OG | SER | B2042 | −17.449 | 39.989 | 11.399 | 1.00 | 42.55 | O | |
| ATOM | 1220 | C | SER | B2O42 | −14.588 | 39.596 | 9.033 | 1.00 | 40.43 | C | |
| ATOM | 1221 | O | SER | B2042 | −14.039 | 40.686 | 9.218 | 1.00 | 40.73 | O | |
| ATOM | 1222 | N | LEU | B2043 | −14.031 | 38.615 | 6.327 | 1.00 | 39.91 | N | |
| ATOM | 1223 | CA | LEU | B2043 | −12.698 | 38.764 | 7.758 | 1.00 | 39.34 | C | |
| ATOM | 1224 | CB | LEU | B2043 | −11.663 | 38.115 | 8.683 | 1.00 | 38.78 | C | |
| ATOM | 1225 | CG | LEU | B2043 | −11.484 | 38.654 | 10.105 | 1.00 | 38.39 | C | |
| ATOM | 1226 | CD1 | LEU | B2043 | −10.642 | 37.666 | 10.913 | 1.00 | 37.70 | C | |
| ATOM | 1227 | CD2 | LEU | B2043 | −10.822 | 40.035 | 10.075 | 1.00 | 37.83 | C | |
| ATOM | 1228 | C | LEU | B2043 | −12.555 | 38.165 | 6.360 | 1.00 | 39.45 | C | |
| ATOM | 1229 | O | LEU | B2043 | −11.502 | 38.317 | 5.732 | 1.00 | 39.47 | O | |
| ATOM | 1230 | N | LYS | B2044 | −13.594 | 37.478 | 5.884 | 1.00 | 38.98 | N | |
| ATOM | 1231 | CA | LYS | B2044 | −13.569 | 36.850 | 4.556 | 1.00 | 38.77 | C | |
| ATOM | 1232 | CB | LYS | B2044 | −14.875 | 37.145 | 3.807 | 1.00 | 39.18 | C | |
| ATOM | 1233 | CG | LYS | B2044 | −14.988 | 36.486 | 2.442 | 0.05 | 38.97 | C | |
| ATOM | 1234 | CD | LYS | B2044 | −15.072 | 34.973 | 2.556 | 0.05 | 38.99 | C | |
| ATOM | 1235 | CE | LYS | B2044 | −15.263 | 34.332 | 1.191 | 0.05 | 38.96 | C | |
| ATOM | 1236 | NZ | LYS | B2044 | −15.382 | 32.851 | 1.284 | 0.05 | 38.96 | N | |
| ATOM | 1237 | C | LYS | B2044 | −12.383 | 37.340 | 3.720 | 1.00 | 38.31 | C | |
| ATOM | 1238 | O | LYS | B2044 | −12.253 | 38.538 | 3.458 | 1.00 | 38.14 | O | |
| ATOM | 1239 | N | GLY | B2045 | −11.522 | 36.412 | 3.310 | 1.00 | 37.92 | N | |
| ATOM | 1240 | CA | GLY | B2045 | −10.351 | 36.767 | 2.521 | 1.00 | 36.95 | C | |
| ATOM | 1241 | C | GLY | B2045 | −9.108 | 36.124 | 3.108 | 1.00 | 36.64 | C | |
| ATOM | 1242 | O | GLY | B2045 | −9.207 | 35.141 | 3.845 | 1.00 | 37.08 | O | |
| ATOM | 1243 | N | ARG | B2046 | −7.935 | 36.665 | 2.607 | 1.00 | 35.68 | N | |
| ATOM | 1244 | CA | ARG | B2046 | −6.703 | 36.084 | 3.332 | 1.00 | 35.35 | C | |
| ATOM | 1245 | CB | ARG | B2046 | −5.476 | 36.804 | 2.750 | 1.00 | 35.15 | C | |
| ATOM | 1246 | CG | ARG | B2046 | −4.150 | 36.148 | 3.087 | 0.05 | 35.14 | C | |
| ATOM | 1247 | CD | ARG | B2046 | −4.081 | 34.743 | 2.515 | 0.05 | 35.11 | C | |
| ATOM | 1248 | NE | ARG | B2046 | −2.811 | 34.090 | 2.811 | 0.05 | 35.11 | N | |
| ATOM | 1249 | CZ | ARG | B2046 | −2.504 | 32.853 | 2.436 | 0.05 | 35.09 | C | |
| ATOM | 1250 | NH1 | ARG | B2046 | −1.323 | 32.338 | 2.748 | 0.05 | 35.09 | N | |
| ATOM | 1251 | NH2 | ARG | B2046 | −3.377 | 32.130 | 1.748 | 0.05 | 35.07 | N | |
| ATOM | 1252 | C | ARG | B2046 | −6.661 | 36.147 | 4.859 | 1.00 | 34.86 | C | |
| ATOM | 1253 | O | ARG | B2046 | −6.273 | 35.184 | 5.522 | 1.00 | 34.07 | O | |
| ATOM | 1254 | N | ARG | B2047 | −7.068 | 37.289 | 5.407 | 1.00 | 34.76 | N | |
| ATOM | 1255 | CA | ARG | B2047 | −7.074 | 37.503 | 6.852 | 1.00 | 35.00 | C | |
| ATOM | 1256 | CB | ARG | B2047 | −7.559 | 38.918 | 7.156 | 1.00 | 35.75 | C | |
| ATOM | 1257 | CG | ARG | B2047 | −6.762 | 39.984 | 6.434 | 1.00 | 37.57 | C | |
| ATOM | 1258 | CD | ARG | B2047 | −7.345 | 41.342 | 6.697 | 1.00 | 39.01 | C | |
| ATOM | 1259 | NE | ARG | B2047 | −8.743 | 41.398 | 6.289 | 1.00 | 40.76 | N | |
| ATOM | 1260 | CZ | ARG | B2047 | −9.663 | 42.124 | 6.915 | 1.00 | 41.64 | C | |
| ATOM | 1261 | NH1 | ARG | B2047 | −9.322 | 42.852 | 7.974 | 1.00 | 41.91 | N | |
| ATOM | 1262 | NH2 | ARG | B2047 | −10.924 | 42.110 | 6.497 | 1.00 | 42.15 | N | |
| ATOM | 1263 | C | ARG | B2047 | −7.959 | 36.483 | 7.566 | 1.00 | 34.24 | C | |
| ATOM | 1264 | O | ARG | B2047 | −7.681 | 36;070 | 8.692 | 1.00 | 34.10 | O | |
| ATOM | 1265 | N | GLN | B2048 | −9.030 | 36.085 | 6.900 | 1.00 | 33.32 | N | |
| ATOM | 1266 | CA | GLN | B2048 | −9.944 | 35.112 | 7.460 | 1.00 | 32.27 | C | |
| ATOM | 1267 | CB | GLN | B2048 | −11.138 | 34.961 | 6.531 | 1.00 | 32.92 | C | |
| ATOM | 1268 | CG | GLN | B2048 | −12.281 | 34.193 | 7.115 | 1.00 | 33.21 | C | |
| ATOM | 1269 | CD | GLN | B2048 | −13.323 | 33.871 | 6.077 | 1.00 | 33.65 | C | |
| ATOM | 1270 | OE1 | GLN | B2048 | −13.016 | 33.274 | 5.044 | 1.00 | 34.07 | O | |
| ATOM | 1271 | NE2 | GLN | B2048 | −14.567 | 34.258 | 6.343 | 1.00 | 33.98 | N | |
| ATOM | 1272 | C | GLN | B2048 | −9.224 | 33.772 | 7.620 | 1.00 | 31.20 | C | |
| ATOM | 1273 | O | GLN | B2048 | −9.402 | 33.075 | 8.615 | 1.00 | 30.73 | O | |
| ATOM | 1274 | N | ILE | B2049 | −8.405 | 33.419 | 6.634 | 1.00 | 29.96 | N | |
| ATOM | 1275 | CA | ILE | B2049 | −7.661 | 32.170 | 6.682 | 1.00 | 28.94 | C | |
| ATOM | 1276 | CB | ILE | B2049 | −7.007 | 31.856 | 5.313 | 1.00 | 28.51 | C | |
| ATOM | 1277 | CG2 | ILE | B2049 | −6.069 | 30.658 | 5.438 | 1.00 | 27.59 | C | |
| ATOM | 1278 | CG1 | ILE | B2049 | −8.096 | 31.573 | 4.267 | 1.00 | 28.47 | C | |
| ATOM | 1279 | CD1 | ILE | B2049 | −8.950 | 30.333 | 4.553 | 1.00 | 27.80 | C | |
| ATOM | 1280 | C | ILE | B2049 | −6.574 | 32.198 | 7.759 | 1.00 | 28.73 | C | |
| ATOM | 1281 | O | ILE | B2049 | −6.353 | 31.206 | 8.463 | 1.00 | 28.34 | O | |
| ATOM | 1282 | N | GLU | B2050 | −5.896 | 33.336 | 7.879 | 1.00 | 28.38 | N | |
| ATOM | 1283 | CA | GLU | B2050 | −4.833 | 33.494 | 8.859 | 1.00 | 28.07 | C | |
| ATOM | 1284 | CB | GLU | B2050 | −4.151 | 34.848 | 8.665 | 1.00 | 29.65 | C | |
| ATOM | 1285 | CG | GLU | B2050 | −3.799 | 35.103 | 7.200 | 1.00 | 32.92 | C | |
| ATOM | 1286 | CD | GLU | B2050 | −2.865 | 36.279 | 6.995 | 1.00 | 34.62 | C | |
| ATOM | 1287 | OE1 | GLU | B2050 | −3.232 | 37.416 | 7.392 | 1.00 | 35.24 | O | |
| ATOM | 1288 | OE2 | GLU | B2050 | −1.762 | 36.055 | 6.430 | 1.00 | 35.19 | O | |
| ATOM | 1289 | C | GLU | B2050 | −5.416 | 33.374 | 10.260 | 1.00 | 26.78 | C | |
| ATOM | 1290 | O | GLU | B2050 | −4.812 | 32.768 | 11.150 | 1.00 | 26.09 | O | |
| ATOM | 1291 | N | TYR | B2051 | −6.600 | 33.944 | 10.453 | 1.00 | 25.41 | N | |
| ATOM | 1292 | CA | TYR | B2051 | −7.251 | 33.868 | 11.749 | 1.00 | 24.34 | C | |
| ATOM | 1293 | CB | TYR | B2051 | −8.535 | 34.703 | 11.741 | 1.00 | 23.37 | C | |
| ATOM | 1294 | CG | TYR | B2051 | −9.316 | 34.618 | 13.024 | 1.00 | 22.77 | C | |
| ATOM | 1295 | CD1 | TYR | B2051 | −10.347 | 33.695 | 13.169 | 1.00 | 22.86 | C | |
| ATOM | 1296 | CE1 | TYR | B2051 | −11.026 | 33.559 | 14.369 | 1.00 | 23.29 | C | |
| ATOM | 1297 | CD2 | TYR | B2051 | −8.984 | 35.413 | 14.116 | 1.00 | 22.81 | C | |
| ATOM | 1298 | CE2 | TYR | B2051 | −9.657 | 35.284 | 15.332 | 1.00 | 23.14 | C | |
| ATOM | 1299 | CZ | TYR | B2051 | −10.677 | 34.350 | 15.450 | 1.00 | 23.43 | C | |
| ATOM | 1300 | OH | TYR | B2051 | −11.332 | 34.186 | 16.649 | 1.00 | 23.03 | O | |
| ATOM | 1301 | C | TYR | B2051 | −7.550 | 32.400 | 12.086 | 1.00 | 24.05 | C | |
| ATOM | 1302 | O | TYR | B2051 | −7.176 | 31.910 | 13.154 | 1.00 | 24.52 | O | |
| ATOM | 1303 | N | LEU | B2052 | −8.211 | 31.700 | 11.170 | 1.00 | 23.25 | N | |
| ATOM | 1304 | CA | LEU | B2052 | −8.541 | 30.291 | 11.364 | 1.00 | 22.91 | C | |
| ATOM | 1305 | CB | LEU | B2052 | −9.274 | 29.748 | 10.133 | 1.00 | 22.55 | C | |
| ATOM | 1306 | CG | LEU | B2052 | −9.598 | 28.247 | 10.086 | 1.00 | 22.85 | C | |
| ATOM | 1307 | CD1 | LEU | B2052 | −10.345 | 27.814 | 11.343 | 1.00 | 21.95 | C | |
| ATOM | 1308 | CD2 | LEU | B2052 | −10.436 | 27.957 | 8.835 | 1.00 | 22.89 | C | |
| ATOM | 1309 | C | LEU | B2052 | −7.284 | 29.457 | 11.622 | 1.00 | 22.68 | C | |
| ATOM | 1310 | O | LEU | B2052 | −7.232 | 28.681 | 12.571 | 1.00 | 22.94 | O | |
| ATOM | 1311 | N | ALA | B2053 | −6.285 | 29.612 | 10.761 | 1.00 | 22.31 | N | |
| ATOM | 1312 | CA | ALA | B2053 | −5.024 | 28.897 | 10.884 | 1.00 | 21.99 | C | |
| ATOM | 1313 | CB | ALA | B2053 | −4.072 | 29.332 | 9.774 | 1.00 | 21.32 | C | |
| ATOM | 1314 | C | ALA | B2053 | −4.397 | 29.180 | 12.244 | 1.00 | 22.13 | C | |
| ATOM | 1315 | O | ALA | B2053 | −3.805 | 28.295 | 12.868 | 1.00 | 21.87 | O | |
| ATOM | 1316 | N | GLY | B2054 | −4.532 | 30.427 | 12.688 | 1.00 | 21.95 | N | |
| ATOM | 1317 | CA | GLY | B2054 | −3.981 | 30.829 | 13.963 | 1.00 | 21.46 | C | |
| ATOM | 1318 | C | GLY | B2054 | −4.654 | 30.111 | 15.107 | 1.00 | 21.35 | C | |
| ATOM | 1319 | O | GLY | B2054 | −3.986 | 29.678 | 16.049 | 1.00 | 21.19 | O | |
| ATOM | 1320 | N | ARG | B2055 | −5.914 | 29.976 | 15.040 | 1.00 | 20.89 | N | |
| ATOM | 1321 | CA | ARG | B2055 | −6.681 | 29.290 | 16.114 | 1.00 | 20.89 | C | |
| ATOM | 1322 | CB | ARG | B2055 | −8.191 | 29.543 | 16.034 | 1.00 | 20.27 | C | |
| ATOM | 1323 | CG | ARG | B2055 | −8.685 | 30.688 | 16.934 | 1.00 | 19.89 | C | |
| ATOM | 1324 | CD | ARG | B2055 | −7.926 | 31.986 | 16.664 | 1.00 | 20.08 | C | |
| ATOM | 1325 | NE | ARG | B2055 | −8.265 | 33.046 | 17.609 | 1.00 | 19.79 | N | |
| ATOM | 1326 | CZ | ARG | B2055 | −7.634 | 34.216 | 17.668 | 1.00 | 20.91 | C | |
| ATOM | 1327 | NH1 | ARG | B2055 | −6.626 | 34.473 | 16.832 | 1.00 | 20.64 | N | |
| ATOM | 1328 | NH2 | ARG | B2055 | −8.009 | 35.135 | 18.555 | 1.00 | 20.13 | N | |
| ATOM | 1329 | C | ARG | B2055 | −6.383 | 27.796 | 16.086 | 1.00 | 20.73 | C | |
| ATOM | 1330 | O | ARG | B2055 | −6.239 | 27.169 | 17.136 | 1.00 | 20.29 | O | |
| ATOM | 1331 | N | TRP | B2056 | −6.273 | 27.235 | 14.884 | 1.00 | 20.69 | N | |
| ATOM | 1332 | CA | TRP | B2056 | −5.976 | 25.817 | 14.742 | 1.00 | 20.72 | C | |
| ATOM | 1333 | CB | TRP | B2056 | −5.992 | 25.408 | 13.262 | 1.00 | 21.90 | C | |
| ATOM | 1334 | CG | TRP | B2056 | −5.402 | 24.051 | 13.020 | 1.00 | 23.77 | C | |
| ATOM | 1335 | CD2 | TRP | B2056 | −6.116 | 22.814 | 12.903 | 1.00 | 24.30 | C | |
| ATOM | 1336 | CE2 | TRP | B2056 | −5.151 | 21.780 | 12.814 | 1.00 | 24.91 | C | |
| ATOM | 1337 | CE3 | TRP | B2056 | −7.473 | 22.478 | 12.869 | 1.00 | 24.91 | C | |
| ATOM | 1338 | CD1 | TRP | B2056 | −4.065 | 23.727 | 12.983 | 1.00 | 23.97 | C | |
| ATOM | 1339 | NE1 | TRP | B2056 | −3.912 | 22.365 | 12.864 | 1.00 | 24.86 | N | |
| ATOM | 1340 | CZ2 | TRP | B2056 | −5.505 | 20.429 | 12.697 | 1.00 | 25.50 | C | |
| ATOM | 1341 | CZ3 | TRP | B2056 | −7.829 | 21.131 | 12.751 | 1.00 | 26.26 | C | |
| ATOM | 1342 | CH2 | TRP | B2056 | −6.843 | 20.122 | 12.667 | 1.00 | 26.22 | C | |
| ATOM | 1343 | C | TRP | B2056 | −4.603 | 25.547 | 15.348 | 1.00 | 20.41 | C | |
| ATOM | 1344 | O | TRP | B2056 | −4.409 | 24.582 | 16.093 | 1.00 | 20.04 | O | |
| ATOM | 1345 | N | SER | B2057 | −3.654 | 26.415 | 15.025 | 1.00 | 19.64 | N | |
| ATOM | 1346 | CA | SER | B2057 | −2.298 | 26.282 | 15.533 | 1.00 | 19.83 | C | |
| ATOM | 1347 | CB | SER | B2057 | −1.395 | 27.339 | 14.898 | 1.00 | 20.26 | C | |
| ATOM | 1348 | OG | SER | B2057 | −0.057 | 27.136 | 15.303 | 1.00 | 22.97 | O | |
| ATOM | 1349 | C | SER | B2057 | −2.239 | 26.402 | 17.062 | 1.00 | 18.86 | C | |
| ATOM | 1350 | O | SER | B2057 | −1.548 | 25.636 | 17.725 | 1.00 | 18.27 | O | |
| ATOM | 1351 | N | ALA | B2058 | −2.968 | 27.367 | 17.613 | 1.00 | 18.16 | N | |
| ATOM | 1352 | CA | ALA | B2058 | −2.996 | 27.566 | 19.058 | 1.00 | 17.01 | C | |
| ATOM | 1353 | CB | ALA | B2058 | −3.767 | 28.826 | 19.388 | 1.00 | 16.89 | C | |
| ATOM | 1354 | C | ALA | B2058 | −3.623 | 26.365 | 19.781 | 1.00 | 16.76 | C | |
| ATOM | 1355 | O | ALA | B2058 | −3.087 | 25.890 | 20.783 | 1.00 | 15.81 | O | |
| ATOM | 1356 | N | LYS | B2059 | −4.755 | 25.884 | 19.272 | 1.00 | 16.40 | N | |
| ATOM | 1357 | CA | LYS | B2059 | −5.446 | 24.746 | 19.883 | 1.00 | 17.29 | C | |
| ATOM | 1358 | CB | LYS | B2059 | −6.815 | 24.547 | 19.217 | 1.00 | 16.13 | C | |
| ATOM | 1359 | CG | LYS | B2059 | −7.751 | 25.730 | 19.498 | 1.00 | 15.32 | C | |
| ATOM | 1360 | CD | LYS | B2059 | −9.168 | 25.536 | 18.996 | 1.00 | 14.90 | C | |
| ATOM | 1361 | CE | LYS | B2059 | −10.011 | 26.783 | 19.319 | 1.00 | 13.82 | C | |
| ATOM | 1362 | NZ | LYS | B2059 | −11.432 | 26.637 | 18.937 | 1.00 | 11.65 | N | |
| ATOM | 1363 | C | LYS | B2059 | −4.620 | 23.464 | 19.826 | 1.00 | 17.91 | C | |
| ATOM | 1364 | O | LYS | B2059 | −4.640 | 22.656 | 20.748 | 1.00 | 16.96 | O | |
| ATOM | 1365 | N | GLU | B2060 | −3.879 | 23.293 | 18.738 | 1.00 | 19.43 | N | |
| ATOM | 1366 | CA | GLU | B2060 | −3.043 | 22.123 | 18.585 | 1.00 | 20.86 | C | |
| ATOM | 1367 | CB | GLU | B2060 | −2.516 | 22.046 | 17.146 | 1.00 | 22.77 | C | |
| ATOM | 1368 | CG | GLU | B2060 | −1.964 | 20.691 | 16.759 | 1.00 | 26.37 | C | |
| ATOM | 1369 | CD | GLU | B2060 | −2.982 | 19.574 | 16.959 | 1.00 | 29.21 | C | |
| ATOM | 1370 | OE1 | GLU | B2060 | −4.047 | 19.602 | 16.287 | 1.00 | 29.92 | O | |
| ATOM | 1371 | OE2 | GLU | B2060 | −2.720 | 18.670 | 17.795 | 1.00 | 30.31 | O | |
| ATOM | 1372 | C | GLU | B2060 | −1.889 | 22.256 | 19.593 | 1.00 | 20.86 | C | |
| ATOM | 1373 | O | GLU | B2060 | −1.556 | 21.302 | 20.307 | 1.00 | 20.96 | O | |
| ATOM | 1374 | N | ALA | B2061 | −1.299 | 23.447 | 19.661 | 1.00 | 20.25 | N | |
| ATOM | 1375 | CA | ALA | B2061 | −0.191 | 23,697 | 20.580 | 1.00 | 20.38 | C | |
| ATOM | 1376 | CB | ALA | B2061 | 0.348 | 25.139 | 20.411 | 1.00 | 19.50 | C | |
| ATOM | 1377 | C | ALA | B2061 | −0.655 | 23.464 | 22.015 | 1.00 | 20.28 | C | |
| ATOM | 1378 | O | ALA | B2061 | 0.103 | 22.952 | 22.841 | 1.00 | 20.05 | O | |
| ATOM | 1379 | N | PHE | B2062 | −1.896 | 23.832 | 22.313 | 1.00 | 20.49 | N | |
| ATOM | 1380 | CA | PHE | B2062 | −2.419 | 23.620 | 23.659 | 1.00 | 21.41 | C | |
| ATOM | 1381 | CB | PHE | B2062 | −3.738 | 24.382 | 23.880 | 1.00 | 21.26 | C | |
| ATOM | 1382 | CG | PHE | B2062 | −4.350 | 24.142 | 25.242 | 1.00 | 21.63 | C | |
| ATOM | 1383 | CD1 | PHE | B2062 | −5.134 | 23.015 | 25.481 | 1.00 | 21.14 | C | |
| ATOM | 1384 | CD2 | PHE | B2062 | −4.092 | 25.009 | 26.297 | 1.00 | 21.77 | C | |
| ATOM | 1385 | CE1 | PHE | B2062 | −5.646 | 22.756 | 26.745 | 1.00 | 21.47 | C | |
| ATOM | 1386 | CE2 | PHE | B2062 | −4.603 | 24.757 | 27.576 | 1.00 | 22.09 | C | |
| ATOM | 1387 | CZ | PHE | B2062 | −5.382 | 23.627 | 27.799 | 1.00 | 21.52 | C | |
| ATOM | 1388 | C | PHE | B2062 | −2.630 | 22.133 | 23.938 | 1.00 | 22.03 | C | |
| ATOM | 1389 | O | PHE | B2062 | −2.303 | 21.655 | 25.022 | 1.00 | 22.28 | O | |
| ATOM | 1390 | N | SER | B2063 | −3.183 | 21.406 | 22.970 | 1.00 | 22.94 | N | |
| ATOM | 1391 | CA | SER | B2063 | −3.403 | 19.972 | 23.135 | 1.00 | 24.34 | C | |
| ATOM | 1392 | CB | SER | B2063 | −4.120 | 19.379 | 21.921 | 1.00 | 24.96 | C | |
| ATOM | 1393 | OG | SER | B2063 | −5.510 | 19.618 | 21.995 | 1.00 | 26.96 | O | |
| ATOM | 1394 | C | SER | B2063 | −2.089 | 19.231 | 23.338 | 1.00 | 24.82 | C | |
| ATOM | 1395 | O | SER | B2063 | −2.053 | 18.192 | 23.989 | 1.00 | 24.78 | O | |
| ATOM | 1396 | N | LYS | B2064 | −1.010 | 19.754 | 22.778 | 1.00 | 24.97 | N | |
| ATOM | 1397 | CA | LYS | B2064 | 0.266 | 19.091 | 22.952 | 1.00 | 26.19 | C | |
| ATOM | 1398 | CB | LYS | B2064 | 1.201 | 19.454 | 21.802 | 1.00 | 25.92 | C | |
| ATOM | 1399 | CG | LYS | B2064 | 0.723 | 18.846 | 20.496 | 1.00 | 26.10 | C | |
| ATOM | 1400 | CD | LYS | B2064 | 1.437 | 19.406 | 19.293 | 1.00 | 26.40 | C | |
| ATOM | 1401 | CE | LYS | B2064 | 2.890 | 19.016 | 19.272 | 1.00 | 25.79 | C | |
| ATOM | 1402 | NZ | LYS | B2064 | 3.444 | 19.339 | 17.935 | 1.00 | 26.57 | N | |
| ATOM | 1403 | C | LYS | B2064 | 0.860 | 19.447 | 24.312 | 1.00 | 27.18 | C | |
| ATOM | 1404 | O | LYS | B2064 | 1.443 | 18.593 | 24.978 | 1.00 | 27.11 | O | |
| ATOM | 1405 | N | ALA | B2065 | 0.680 | 20.696 | 24.734 | 1.00 | 28.10 | N | |
| ATOM | 1406 | CA | ALA | B2065 | 1.175 | 21.142 | 26.030 | 1.00 | 29.62 | C | |
| ATOM | 1407 | CB | ALA | B2065 | 0.851 | 22.617 | 26.241 | 1.00 | 29.09 | C | |
| ATOM | 1408 | C | ALA | B2065 | 0.520 | 20.309 | 27.128 | 1.00 | 30.86 | C | |
| ATOM | 1409 | O | ALA | B2065 | 1.121 | 20.062 | 28.170 | 1.00 | 30.13 | O | |
| ATOM | 1410 | N | MET | B2066 | −0.721 | 19.890 | 26.892 | 1.00 | 32.95 | N | |
| ATOM | 1411 | CA | MET | B2066 | −1.449 | 19.079 | 27.866 | 1.00 | 35.41 | C | |
| ATOM | 1412 | CB | MET | B2066 | −2.957 | 19.167 | 27.626 | 1.00 | 35.78 | C | |
| ATOM | 1413 | CG | MET | B2066 | −3.588 | 20.479 | 28.047 | 1.00 | 36.88 | C | |
| ATOM | 1414 | SD | MET | B2066 | −3.734 | 20.673 | 29.828 | 1.00 | 38.18 | S | |
| ATOM | 1415 | CE | MET | B2066 | −5.497 | 20.288 | 30.048 | 1.00 | 38.92 | C | |
| ATOM | 1416 | C | MET | B2066 | −1.010 | 17.621 | 27.783 | 1.00 | 36.81 | C | |
| ATOM | 1417 | O | MET | B2066 | −1.414 | 16.797 | 28.595 | 1.00 | 36.82 | O | |
| ATOM | 1418 | N | GLY | B2067 | −0.178 | 17.311 | 26.798 | 1.00 | 38.52 | N | |
| ATOM | 1419 | CA | GLY | B2067 | −0.292 | 15.950 | 26.644 | 1.00 | 41.07 | C | |
| ATOM | 1420 | C | GLY | B2067 | −0.794 | 14.998 | 26.187 | 1.00 | 42.84 | C | |
| ATOM | 1421 | O | GLY | B2067 | −1.321 | 14.214 | 26.979 | 1.00 | 43.50 | O | |
| ATOM | 1422 | N | THR | B2068 | −1.140 | 15.077 | 24.908 | 1.00 | 44.30 | N | |
| ATOM | 1423 | CA | THR | B2068 | −2.152 | 14.206 | 24.322 | 1.00 | 45.93 | C | |
| ATOM | 1424 | CB | THP | B2068 | −3.410 | 14.994 | 23.903 | 1.00 | 45.92 | C | |
| ATOM | 1425 | OG1 | THR | B2068 | −3.075 | 15.921 | 22.861 | 1.00 | 46.12 | O | |
| ATOM | 1426 | CG2 | THR | B2068 | −3.972 | 15.758 | 25.093 | 1.00 | 46.09 | C | |
| ATOM | 1427 | C | THP | B2068 | −1.501 | 13.593 | 23.088 | 1.00 | 47.07 | C | |
| ATOM | 1428 | O | THR | B2068 | −2.151 | 12.918 | 22.287 | 1.00 | 47.09 | O | |
| ATOM | 1429 | N | GLY | B2069 | −0.202 | 13.862 | 22.957 | 1.00 | 48.53 | N | |
| ATOM | 1430 | CA | GLY | B2069 | 0.599 | 13.356 | 21.853 | 1.00 | 49.83 | C | |
| ATOM | 1431 | C | GLY | B2069 | −0.085 | 13.307 | 20.503 | 1.00 | 50.67 | C | |
| ATOM | 1432 | O | GLY | B2069 | −0.047 | 14.274 | 19.741 | 1.00 | 51.10 | O | |
| ATOM | 1433 | N | ILE | B2070 | −0.699 | 12.166 | 20.208 | 1.00 | 51.17 | N | |
| ATOM | 1434 | CA | ILE | B2070 | −1.405 | 11.948 | 18.952 | 1.00 | 51.53 | C | |
| ATOM | 1435 | CB | ILE | B2070 | −1.574 | 10.427 | 18.668 | 1.00 | 51.94 | C | |
| ATOM | 1436 | CG2 | ILE | B2070 | −0.221 | 9.804 | 18.341 | 1.00 | 51.87 | C | |
| ATOM | 1437 | CG1 | ILE | B2070 | −2.217 | 9.737 | 19.882 | 1.00 | 52.15 | C | |
| ATOM | 1438 | CD1 | ILE | B2070 | −2.470 | 8.249 | 19.712 | 1.00 | 52.43 | C | |
| ATOM | 1439 | C | ILE | B2070 | −2.792 | 12.590 | 18.979 | 1.00 | 51.42 | C | |
| ATOM | 1440 | O | ILE | B2070 | −3.074 | 13.534 | 18.232 | 1.00 | 51.57 | O | |
| ATOM | 1441 | N | SER | B2071 | −3.653 | 12.068 | 19.847 | 1.00 | 51.02 | N | |
| ATOM | 1442 | CA | SER | B2071 | −5.013 | 12.566 | 19.967 | 1.00 | 50.61 | C | |
| ATOM | 1443 | CB | SER | B2071 | −5.803 | 11.680 | 20.938 | 1.00 | 50.61 | C | |
| ATOM | 1444 | OG | SER | B2071 | −5.007 | 11.299 | 22.044 | 1.00 | 50.73 | O | |
| ATOM | 1445 | C | SER | B2071 | −5.064 | 14.030 | 20.404 | 1.00 | 50.07 | C | |
| ATOM | 1446 | O | SER | B2071 | −4.042 | 14.625 | 20.771 | 1.00 | 49.91 | O | |
| ATOM | 1447 | N | LYS | B2072 | −6.258 | 14.612 | 20.340 | 1.00 | 49.03 | N | |
| ATOM | 1448 | CA | LYS | B2072 | −6.440 | 16.004 | 20.728 | 1.00 | 47.71 | C | |
| ATOM | 1449 | CB | LYS | B2072 | −6.637 | 16.898 | 19.495 | 1.00 | 48.58 | C | |
| ATOM | 1450 | CG | LYS | B2072 | −5.733 | 16.397 | 18.312 | 1.00 | 49.59 | C | |
| ATOM | 1451 | CD | LYS | B2072 | −6.215 | 17.358 | 17.087 | 1.00 | 50.30 | C | |
| ATOM | 1452 | CE | LYS | B2072 | −5.500 | 16.909 | 15.820 | 1.00 | 50.97 | C | |
| ATOM | 1453 | NZ | LYS | B2072 | −6.051 | 17.588 | 14.610 | 1.00 | 51.33 | N | |
| ATOM | 1454 | C | LYS | B2072 | −7.670 | 16.146 | 21.610 | 1.00 | 46.22 | C | |
| ATOM | 1455 | O | LYS | B2072 | −8.484 | 15.229 | 21.742 | 1.00 | 46.03 | O | |
| ATOM | 1456 | N | LEU | B2073 | −7.787 | 17.322 | 22.208 | 1.00 | 44.70 | N | |
| ATOM | 1457 | CA | LEU | B2073 | −8.915 | 17.658 | 23.048 | 1.00 | 42.68 | C | |
| ATOM | 1458 | CB | LEU | B2073 | −8.540 | 18.812 | 23.975 | 1.00 | 42.47 | C | |
| ATOM | 1459 | CG | LEU | B2073 | −7.416 | 18.504 | 24.959 | 1.00 | 42.08 | C | |
| ATOM | 1460 | CD1 | LEU | B2073 | −6.916 | 19.781 | 25.594 | 1.00 | 42.04 | C | |
| ATOM | 1461 | CD2 | LEU | B2073 | −7.925 | 17.543 | 26.010 | 1.00 | 42.14 | C | |
| ATOM | 1462 | C | LEU | B2073 | −9.999 | 18.116 | 22.083 | 1.00 | 41.35 | C | |
| ATOM | 1463 | O | LEU | B2073 | −9.750 | 18.273 | 20.881 | 1.00 | 41.21 | O | |
| ATOM | 1464 | N | GLY | B2074 | −11.203 | 18.312 | 22.595 | 1.00 | 39.49 | N | |
| ATOM | 1465 | CA | GLY | B2074 | −12.252 | 18.791 | 21.727 | 1.00 | 37.51 | C | |
| ATOM | 1466 | C | GLY | B2074 | −11.852 | 20.211 | 21.364 | 1.00 | 35.85 | C | |
| ATOM | 1467 | O | GLY | B2074 | −11.493 | 20.998 | 22.240 | 1.00 | 35.43 | O | |
| ATOM | 1468 | N | PHE | B2075 | −11.878 | 20.548 | 20.082 | 1.00 | 33.89 | N | |
| ATOM | 1469 | CA | PHE | B2075 | −11.524 | 21.900 | 19.704 | 1.00 | 32.41 | C | |
| ATOM | 1470 | CB | PHE | B2075 | −11.287 | 21.990 | 18.194 | 1.00 | 32.43 | C | |
| ATOM | 1471 | CG | PHE | B2075 | −9.846 | 21.778 | 17.802 | 1.00 | 32.87 | C | |
| ATOM | 1472 | CD1 | PHE | B2075 | −8.952 | 21.151 | 18.682 | 1.00 | 33.28 | C | |
| ATOM | 1473 | CD2 | PHE | B2075 | −9.379 | 22.191 | 16.557 | 1.00 | 33.06 | C | |
| ATOM | 1474 | CE1 | PHE | B2075 | −7.616 | 20.941 | 18.326 | 1.00 | 33.27 | C | |
| ATOM | 1475 | CE2 | PHE | B2075 | −8.047 | 21.987 | 16.187 | 1.00 | 33.20 | C | |
| ATOM | 1476 | CZ | PHE | B2075 | −7.163 | 21.360 | 17.071 | 1.00 | 33.45 | C | |
| ATOM | 1477 | C | PHE | B2075 | −12.597 | 22.877 | 20.163 | 1.00 | 31.04 | C | |
| ATOM | 1478 | O | PHE | B2075 | −12.340 | 24.072 | 20.277 | 1.00 | 30.78 | O | |
| ATOM | 1479 | N | GLN | B2076 | −13.787 | 22.357 | 20.462 1.00 | 29.52 | N | | |
| ATOM | 1480 | CA | GLN | B2076 | −14.897 | 23.185 | 20.929 1.00 | 28.06 | C | | |
| ATOM | 1481 | CB | GLN | B2076 | −16.226 | 22.493 | 20.630 1.00 | 27.83 | C | | |
| ATOM | 1482 | CG | GLN | B2076 | −16.489 | 22.365 | 19.144 1.00 | 27.93 | C | | |
| ATOM | 1483 | CD | GLN | B2076 | −16.666 | 23.716 | 18.479 1.00 | 28.25 | C | | |
| ATOM | 1484 | OE1 | GLN | B2076 | −16.170 | 23.950 | 17.376 | 1.00 | 28.52 | O | |
| ATOM | 1485 | NE2 | GLN | B2076 | −17.387 | 24.613 | 19.145 | 1.00 | 27.92 | N | |
| ATOM | 1486 | C | GLN | B2076 | −14.765 | 23.476 | 22.423 | 1.00 | 27.11 | C | |
| ATOM | 1487 | O | GLN | B2076 | −15.536 | 24.241 | 23.000 | 1.00 | 26.54 | O | |
| ATOM | 1488 | N | ASP | B2077 | −13.768 | 22.860 | 23.037 | 1.00 | 26.23 | N | |
| ATOM | 1489 | CA | ASP | B2077 | −13.505 | 23.059 | 24.447 | 1.00 | 26.09 | C | |
| ATOM | 1490 | CB | ASP | B2077 | −13.173 | 21.721 | 25.108 | 1.00 | 27.19 | C | |
| ATOM | 1491 | CG | ASP | B2077 | −14.401 | 20.856 | 25.292 | 1.00 | 28.36 | C | |
| ATOM | 1492 | OD1 | ASP | B2077 | −15.304 | 21.276 | 26.043 | 1.00 | 29.34 | O | |
| ATOM | 1493 | OD2 | ASP | B2077 | −14.469 | 19.767 | 24.686 | 1.00 | 29.83 | O | |
| ATOM | 1494 | C | ASP | B2077 | −12.354 | 24.041 | 24.614 | 1.00 | 24.96 | C | |
| ATOM | 1495 | O | ASP | B2077 | −11.917 | 24.328 | 25.730 | 1.00 | 24.58 | O | |
| ATOM | 1496 | N | LEU | B2078 | −11.870 | 24.553 | 23.490 | 1.00 | 23.44 | N | |
| ATOM | 1497 | CA | LEU | B2078 | −10.783 | 25.507 | 23.502 | 1.00 | 22.63 | C | |
| ATOM | 1498 | CB | LEU | B2078 | −9.534 | 24.900 | 22.868 | 1.00 | 22.62 | C | |
| ATOM | 1499 | CG | LEU | B2078 | −8.989 | 23.599 | 23.468 | 1.00 | 22.75 | C | |
| ATOM | 1500 | CD1 | LEU | B2078 | −7.933 | 23.010 | 22.543 | 1.00 | 22.77 | C | |
| ATOM | 1501 | CD2 | LEU | B2078 | −8.401 | 23.870 | 24.841 | 1.00 | 22.96 | C | |
| ATOM | 1502 | C | LEU | B2078 | −11.200 | 26.741 | 22.726 | 1.00 | 22.37 | C | |
| ATOM | 1503 | O | LEU | B2078 | −11.709 | 26.642 | 21.611 | 1.00 | 21.73 | O | |
| ATOM | 1504 | N | GLU | B2079 | −11.001 | 27.908 | 23.324 | 1.00 | 21.99 | N | |
| ATOM | 1505 | CA | GLU | B2079 | −11.342 | 29.145 | 22.648 | 1.00 | 21.60 | C | |
| ATOM | 1506 | CB | GLU | B2079 | −12.624 | 29.740 | 23.214 | 1.00 | 21.69 | C | |
| ATOM | 1507 | CG | GLU | B2079 | −13.132 | 30.922 | 22.421 | 1.00 | 22.30 | C | |
| ATOM | 1508 | CD | GLU | B2079 | −14.459 | 31.412 | 22.937 | 1.00 | 23.08 | C | |
| ATOM | 1509 | OE1 | GLU | B2079 | −14.499 | 31.893 | 24.089 | 1.00 | 24.19 | O | |
| ATOM | 1510 | OE2 | GLU | B2079 | −15.462 | 31.311 | 22.201 | 1.00 | 22.86 | O | |
| ATOM | 1511 | C | GLU | B2079 | −10.216 | 30.144 | 22.792 | 1.00 | 21.14 | C | |
| ATOM | 1512 | O | GLU | B2079 | −9.606 | 30.265 | 23.862 | 1.00 | 21.43 | O | |
| ATOM | 1513 | N | VAL | | B2080 | −9.940 | 30.852 | 21.705 | 1.00 | 20.42 | N |
| ATOM | 1514 | CA | VAL | B2080 | −8.892 | 31.850 | 21.691 | 1.00 | 20.15 | C | |
| ATOM | 1515 | CB | VAL | B2080 | −7.728 | 31.418 | 20.780 | 1.00 | 19.96 | C | |
| ATOM | 1516 | CG1 | VAL | B2080 | −6.662 | 32.517 | 20.735 | 1.00 | 19.66 | C | |
| ATOM | 1517 | CG2 | VAL | B2080 | −7.127 | 30.123 | 21.297 | 1.00 | 19.86 | C | |
| ATOM | 1518 | C | VAL | B2080 | −9.436 | 33.187 | 21.211 | 1.00 | 20.63 | C | |
| ATOM | 1519 | O | VAL | B2080 | −9.899 | 33.820 | 20.075 | 1.00 | 20.55 | O | |
| ATOM | 1520 | N | LEU | B2081 | −9.379 | 34.178 | 22.089 | 1.00 | 20.91 | N | |
| ATOM | 1521 | CA | LEU | B2081 | −9,852 | 35.515 | 21.767 | 1.00 | 21.71 | C | |
| ATOM | 1522 | CB | LEU | B2081 | −10.913 | 35.953 | 22.779 | 1.00 | 21.41 | C | |
| ATOM | 1523 | CG | LEU | B2081 | −12.150 | 35.049 | 22.846 | 1.00 | 21.70 | C | |
| ATOM | 1524 | COl | LEU | B2081 | −13.145 | 35.618 | 23.864 | 1.00 | 20.86 | C | |
| ATOM | 1525 | CG2 | LEU | B2081 | −12.788 | 34.947 | 21.451 | 1.00 | 20.91 | C | |
| ATOM | 1526 | C | LEU | B2081 | −8.690 | 36.488 | 21.802 | 1.00 | 21.95 | C | |
| ATOM | 1527 | O | LEU | B2081 | −7.592 | 36.135 | 22.255 | 1.00 | 22.11 | O | |
| ATOM | 1528 | N | ASN | B2082 | −8.927 | 37.700 | 21.302 | 1.00 | 22.09 | N | |
| ATOM | 1529 | CA | ASN | B2082 | −7.912 | 38.754 | 21.334 | 1.00 | 22.53 | C | |
| ATOM | 1530 | CB | ASN | B2082 | −7.809 | 39.483 | 19.992 | 1.00 | 23.53 | C | |
| ATOM | 1531 | CG | ASN | B2082 | −7.347 | 38.588 | 18.882 | 1.00 | 24.84 | C | |
| ATOM | 1532 | 001 | ASN | B2082 | −6.350 | 37.880 | 19.016 | 1.00 | 25.69 | O | |
| ATOM | 1533 | ND2 | ASN | B2082 | −8.066 | 38.613 | 17.766 | 1.00 | 26.27 | N | |
| ATOM | 1534 | C | ASM B2082 | −8.345 | 39.754 | 22.402 | 1.00 | 22.18 | C | | |
| ATOM | 1535 | O | ASN | B2082 | −9.528 | 40.069 | 22.514 | 1.00 | 21.35 | O | |
| ATOM | 1536 | N | ASN | B2083 | −7.391 | 40.244 | 23.186 | 1.00 | 22.35 | N | |
| ATOM | 1537 | CA | ASN | B2083 | −7.699 | 41.215 | 24.227 | 1.00 | 22.53 | C | |
| ATOM | 1538 | CB | ASN | B2083 | −6.711 | 41.092 | 25.391 | 1.00 | 21.65 | C | |
| ATOM | 1539 | CG | ASN | B2083 | −5.267 | 41.394 | 24.995 | 1.00 | 21.91 | C | |
| ATOM | 1540 | ODi | ASN | B2083 | −4.338 | 41.115 | 25.762 | 1.00 | 22.04 | O | |
| ATOM | 1541 | ND2 | ASN | B2083 | −5.068 | 41.963 | 23.808 | 1.00 | 20.96 | N | |
| ATOM | 1542 | C | ASN | B2083 | −7.679 | 42.632 | 23.663 | 1.00 | 23.31 | C | |
| ATOM | 1543 | O | ASN | B2083 | −7.344 | 42.834 | 22.493 | 1.00 | 23.42 | O | |
| ATOM | 1544 | N | GLU | B2084 | −8.037 | 43.605 | 24.498 | 1.00 | 23.94 | N | |
| ATOM | 1545 | CA | GLU | B2084 | −8.062 | 45.012 | 24.098 | 1.00 | 24.84 | C | |
| ATOM | 1546 | CB | GLU | B2084 | −8.089 | 45.924 | 25.336 | 1.0026.09 | C | | |
| ATOM | 1547 | CG | GLU | B2084 | −9.171 | 45.622 | 26.355 | 1.00 | 28.21 | C | |
| ATOM | 1548 | CD | OLU B2084 | −8.992 | 44.259 | 27.014 | 1.00 | 30.57 | C | | |
| ATOM | 1549 | OE1 | GLU | B2084 | −7.892 | 44.001 | 27.565 | 1.00 | 31.38 | O | |
| ATOM | 1550 | OE2 | GLU | B2084 | −9.952 | 43.448 | 26.984 | 1.00 | 31.36 | O | |
| ATOM | 1551 | C | GLU | B2084 | −6.871 | 45.421 | 23.223 | 1.00 | 24.40 | C | |
| ATOM | 1552 | O | GLU | B2084 | −7.033 | 46.197 | 22.292 | 1.00 | 24.89 | O | |
| ATOM | 1553 | N | ARG | B2085 | −5.683 | 44.905 | 23.530 | 1.00 | 24.24 | N | |
| ATOM | 1554 | CA | ARG | B2085 | −4.464 | 45.245 | 22.782 | 1.00 | 24.07 | C | |
| ATOM | 1555 | CB | ARG | B2085 | −3.237 | 45.139 | 23.688 | 1.00 | 24.81 | C | |
| ATOM | 1556 | CG | ARG | B2085 | −3.375 | 45.824 | 25.022 | 1.00 | 26.63 | C | |
| ATOM | 1557 | CD | ARG | B2085 | −2.090 | 45.727 | 25.844 | 1.00 | 27.63 | C | |
| ATOM | 1558 | NE | ARG | B2085 | −2.124 | 46.668 | 26.962 | 1.00 | 28.81 | N | |
| ATOM | 1559 | CZ | ARG | B2095 | −2.915 | 46.532 | 28.016 | 1.00 | 29.08 | C | |
| ATOM | 1560 | NH1 | ARG | B2085 | −3.725 | 45.489 | 28.090 | 1.00 | 30.38 | N | |
| ATOM | 1561 | NH2 | ARG | B2085 | −2.915 | 47.441 | 28.984 | 1.00 | 29.49 | N | |
| ATOM | 1562 | C | ARG | B2085 | −4.201 | 44.386 | 21.548 | 1.00 | 23.45 | C | |
| ATOM | 1563 | O | ARG | B2085 | −3.231 | 44.616 | 20.839 | 1.00 | 23.48 | O | |
| ATOM | 1564 | N | GLY | B2086 | −5.043 | 43.388 | 21.304 | 1.00 | 22.86 | N | |
| ATOM | 1565 | CA | GLY | B2086 | −4.845 | 42.531 | 20.148 | 1.00 | 22.18 | C | |
| ATOM | 1566 | C | GLY | B2086 | −4.106 | 41.231 | 20.442 | 1.00 | 22.02 | C | |
| ATOM | 1567 | O | GLY | B2086 | −3.935 | 40.391 | 19.557 | 1.00 | 22.97 | O | |
| ATOM | 1568 | N | ALA | B2087 | −3.658 | 41.053 | 21.677 | 1.00 | 20.91 | N | |
| ATOM | 1569 | CA | ALA | B2087 | −2.948 | 39.836 | 22.038 | 1.00 | 19.91 | C | |
| ATOM | 1570 | CB | ALA | B2087 | −2.196 | 40.028 | 23.362 | 1.00 | 19.31 | C | |
| ATOM | 1571 | C | ALA | B2087 | −3.940 | 38.675 | 22.161 | 1.00 | 19.35 | C | |
| ATOM | 1572 | O | ALA | B2087 | −5.013 | 38.816 | 22.757 | 1.00 | 18.31 | O | |
| ATOM | 1573 | N | PRO | B2088 | −3.598 | 37.520 | 21.566 | 1.00 | 19.16 | N | |
| ATOM | 1574 | CB | PRO | B2088 | −2.449 | 37.311 | 20.656 | 1.00 | 19.16 | C | |
| ATOM | 1575 | CA | PRO | B2088 | −4.450 | 36.331 | 21.611 | 1.00 | 18.85 | C | |
| ATOM | 1576 | CB | PRO | B2088 | −3.968 | 35.529 | 20.408 | 1.00 | 19.28 | C | |
| ATOM | 1577 | CG | PRO | B2088 | −2.478 | 35.817 | 20.403 | 1.00 | 18.72 | C | |
| ATOM | 1578 | C | PRO | B2088 | −4.229 | 35.597 | 22.934 | 1.00 | 18.76 | C | |
| ATOM | 1579 | O | PRO | B2088 | −3.098 | 35.484 | 23.404 | 1.00 | 18.99 | O | |
| ATOM | 1580 | N | TYR | B2089 | −5.310 | 35.108 | 23.533 | 1.00 | 18.27 | N | |
| ATOM | 1581 | CA | TYR | B2089 | −5.230 | 34.394 | 24.804 | 1.00 | 17.56 | C | |
| ATOM | 1582 | CB | TYR | B2089 | −5.394 | 35.379 | 25.960 | 1.00 | 15.80 | C | |
| ATOM | 1583 | CG | TYR | B2089 | −6.792 | 35.933 | 26.103 | 1.00 | 14.62 | C | |
| ATOM | 1584 | CD1 | TYR | B2089 | −7.684 | 35.402 | 27.036 | 1.00 | 14.04 | C | |
| ATOM | 1585 | CE1 | TYR | B2089 | −8.983 | 35.916 | 27.160 | 1.00 | 13.45 | C | |
| ATOM | 1586 | CD2 | TYR | B2089 | −7.231 | 36.991 | 25.294 | 1.00 | 13.76 | C | |
| ATOM | 1587 | CE2 | TYR | B2089 | −8.507 | 37.499 | 25.405 | 1.00 | 12.48 | C | |
| ATOM | 1588 | CZ | TYR | B2089 | −9.381 | 36.964 | 26.336 | 1.00 | 13.07 | C | |
| ATOM | 1589 | OH | TYR | B2089 | −10.649 | 37.475 | 26.433 | 1.00 | 12.87 | O | |
| ATOM | 1590 | C | TYR | B2089 | −6.333 | 33.349 | 24.865 | 1.00 | 18.14 | C | |
| ATOM | 1591 | O | TYR | B2089 | −7.302 | 33.433 | 24.118 | 1.00 | 18.32 | O | |
| ATOM | 1592 | N | PHE | B2090 | −6.196 | 32.360 | 25.741 | 1.00 | 18.75 | N | |
| ATOM | 1593 | CA | PHE | B2090 | −7.242 | 31.350 | 25.853 | 1.00 | 19.41 | C | |
| ATOM | 1594 | CB | PHE | B2090 | −6.686 | 30.012 | 26.353 | 1.00 | 18.52 | C | |
| ATOM | 1595 | CG | PHE | B2090 | −6.044 | 29.187 | 25.282 | 1.00 | 18.19 | C | |
| ATOM | 1596 | CD1 | PHE | B2090 | −4.716 | 29.402 | 24.912 | 1.00 | 17.08 | C | |
| ATOM | 1597 | CD2 | PHE | B2090 | 6.769 | 28.182 | 24.638 | 1.00 | 17.63 | C | |
| ATOM | 1598 | CE1 | PHE | B2090 | −4.119 | 28.626 | 23.920 | 1.00 | 16.47 | C | |
| ATOM | 1599 | CE2 | PHE | B2090 | −6.177 | 27.400 | 23.640 | 1.00 | 16.79 | C | |
| ATOM | 1600 | CZ | PHE | B2090 | −4.847 | 27.625 | 23.284 | 1.00 | 16.73 | C | |
| ATOM | 1601 | C | PHE | B2090 | −8.334 | 31.806 | 26.802 | 1.00 | 20.35 | C | |
| ATOM | 1602 | O | PHE | B2090 | −8.092 | 31.963 | 27.997 | 1.00 | 20.62 | O | |
| ATOM | 1603 | N | SER | B2091 | −9.535 | 32.010 | 26.271 | 1.00 | 21.43 | N | |
| ATOM | 1604 | CA | SER | B2091 | −10.664 | 32.430 | 27.096 | 1.00 | 22.72 | C | |
| ATOM | 1605 | CB | SER | B2091 | −11.635 | 33.293 | 26.278 | 1.00 | 22.59 | C | |
| ATOM | 1606 | OG | SER | B2091 | −12.173 | 32.566 | 25.187 | 1.00 | 24.06 | O | |
| ATOM | 1607 | C | SER | B2091 | −11.385 | 31.194 | 27.651 | 1.00 | 23.50 | C | |
| ATOM | 1608 | O | SER | B2091 | −12.133 | 31.289 | 28.623 | 1.00 | 23.39 | O | |
| ATOM | 1609 | N | GLN | B2092 | −11.154 | 30.041 | 27.024 | 1.00 | 24.05 | N | |
| ATOM | 1610 | CA | GLN | B2092 | −11.757 | 28.784 | 27.458 | 1.00 | 25.18 | C | |
| ATOM | 1611 | CB | GLN | B2092 | −12.970 | 28.420 | 26.591 | 1.00 | 26.15 | C | |
| ATOM | 1612 | CG | GLN | B2092 | −14.194 | 29.321 | 26.733 | 1.00 | 28.02 | C | |
| ATOM | 1613 | CD | GLN | B2092 | −14.848 | 29.241 | 28.105 | 1.00 | 29.69 | C | |
| ATOM | 1614 | OE1 | GLN | B2092 | −14.974 | 28.159 | 28.687 | 1.00 | 30.83 | O | |
| ATOM | 1615 | NE2 | GLN | B2092 | −15.283 | 30.389 | 28.622 | 1.00 | 30.35 | N | |
| ATOM | 1616 | C | GLN | 92092 | −10.725 | 27.661 | 27.354 | 1.00 | 25.31 | C | |
| ATOM | 1617 | O | GLN | 92092 | −10.029 | 27.541 | 26.343 | 1.00 | 25.18 | O | |
| ATOM | 1618 | N | ALA | B2093 | −10.631 | 26.838 | 28.395 | 1.00 | 25.61 | N | |
| ATOM | 1619 | CA | ALA | B2093 | −9.680 | 25.733 | 28.394 | 1.00 | 26.50 | C | |
| ATOM | 1620 | CB | ALA | 22093 | −8.243 | 26.278 | 28.384 | 1.00 | 25.87 | C | |
| ATOM | 1621 | C | ALA | 22093 | −9.876 | 24.805 | 29.587 | 1.00 | 27.03 | C | |
| ATOM | 1622 | O | ALA | B2093 | −10.200 | 25.252 | 30.687 | 1.00 | 27.16 | O | |
| ATOM | 1623 | N | PRO | | B2094 | −9.671 | 23.492 | 29.382 | 1.00 | 27.79 | N |
| ATOM | 1624 | CD | PRO | | B2094 | −9.377 | 22.840 | 28.090 | 1.00 | 27.87 | C |
| ATOM | 1625 | CA | PRO | | B2094 | −9.824 | 22.497 | 30.452 | 1.00 | 28.17 | C |
| ATOM | 1626 | CB | PRO | | B2094 | −9.970 | 21.191 | 29.681 | 1.00 | 27.97 | C |
| ATOM | 1627 | CG | PRO | | B2094 | −9.068 | 21.408 | 28.501 | 1.00 | 27.75 | C |
| ATOM | 1628 | C | PRO | | B2094 | −8.611 | 22.489 | 31.384 | 1.00 | 28.93 | C |
| ATOM | 1629 | O | PRO | | B2094 | −8.149 | 21.429 | 31.819 | 1.00 | 29.42 | O |
| ATOM | 1630 | N | PHE | B2095 | −8.099 | 23.676 | 31.690 | 1.00 | 29.04 | N | |
| ATOM | 1631 | CA | PHE | B2095 | −6.929 | 23.812 | 32.551 | 1.00 | 28.93 | C | |
| ATOM | 1632 | CB | PHE | B2095 | −5.704 | 24.150 | 31.689 | 1.00 | 28.61 | C | |
| ATOM | 1633 | CG | PHE | B2095 | −4.416 | 24.266 | 32.460 | 1.00 | 28.08 | C | |
| ATOM | 1634 | CD1 | PHE | B2095 | −3.697 | 25.457 | 32.454 | 1.00 | 27.74 | C | |
| ATOM | 1635 | CG2 | PHE | B2095 | −3.918 | 23.185 | 33.187 | 1.00 | 27.48 | C | |
| ATOM | 1636 | CBl | PHE | B2095 | −2.503 | 25.574 | 33.158 | 1.00 | 27.44 | C | |
| ATOM | 1637 | CB2 | PHE | B2095 | −2.730 | 23.292 | 33.893 | 1.00 | 27.21 | C | |
| ATOM | 1638 | CZ | PHE | B2095 | −2.018 | 24.492 | 33.880 | 1.00 | 27.64 | C | |
| ATOM | 1639 | C | PHE | B2095 | −7.220 | 24.934 | 33.544 | 1.00 | 29.17 | C | |
| ATOM | 1640 | O | PHE | B2095 | −7.922 | 25.893 | 33.211 | 1.00 | 29.61 | O | |
| ATOM | 1641 | N | SER | B2096 | −6.690 | 24.821 | 34.758 | 1.00 | 28.80 | N | |
| ATOM | 1642 | CA | SER | B2096 | −6.931 | 25.837 | 35.771 | 1.00 | 28.67 | C | |
| ATOM | 1643 | CB | SER | B2096 | −7.374 | 25.177 | 37.078 | 1.00 | 29.60 | C | |
| ATOM | 1644 | OG | SER | B2096 | −8.489 | 24.328 | 36.859 | 1.00 | 31.74 | O | |
| ATOM | 1645 | C | SER | B2096 | −5.719 | 26.716 | 36.038 | 1.00 | 27.69 | C | |
| ATOM | 1646 | O | SER | B2096 | −5.828 | 27.746 | 36.700 | 1.00 | 27.77 | O | |
| ATOM | 1647 | N | GLY | B2097 | −4.563 | 26.318 | 35.526 | 1.00 | 26.80 | N | |
| ATOM | 1648 | CA | GLY | B2097 | −3.370 | 27.115 | 35.752 | 1.00 | 25.56 | C | |
| ATOM | 1649 | C | GLY | B2097 | −3.246 | 28.268 | 34.771 | 1.00 | 24.43 | C | |
| ATOM | 1650 | O | GLY | B2097 | −4.224 | 28.686 | 34.155 | 1.00 | 23.36 | O | |
| ATOM | 1651 | N | LYS | B2098 | −2.032 | 28.786 | 34.629 | 1.00 | 23.85 | N | |
| ATOM | 1652 | CA | LYS | B2098 | −1.786 | 29.886 | 33.707 | 1.00 | 23.16 | C | |
| ATOM | 1653 | CB | LYS | B2098 | −0.656 | 30.789 | 34.212 | 1.00 | 24.15 | C | |
| ATOM | 1654 | CG | LYS | B2098 | −1.025 | 31.639 | 35.408 | 1.00 | 25.14 | C | |
| ATOM | 1655 | CD | LYS | B2098 | 0.036 | 32.693 | 35.659 | 1.00 | 26.88 | C | |
| ATOM | 1656 | CE | LYS | B2098 | −0.288 | 33.524 | 36.895 | 1.00 | 27.83 | C | |
| ATOM | 1657 | NZ | LYS | B2098 | −0.224 | 32.680 | 38.126 | 1.00 | 28.96 | N | |
| ATOM | 1658 | C | LYS | B2098 | −1.406 | 29.332 | 32.353 | 1.00 | 21.88 | C | |
| ATOM | 1659 | O | LYS | B2098 | −0.604 | 28.402 | 32.261 | 1.00 | 21.71 | O | |
| ATOM | 1660 | N | ILE | B2099 | −1.997 | 29.905 | 31.310 | 1.00 | 20.39 | N | |
| ATOM | 1661 | CA | ILE | B2099 | −1.723 | 29.494 | 29.941 | 1.00 | 19.37 | C | |
| ATOM | 1662 | CB | ILE | B2099 | −3.027 | 29.186 | 29.165 | 1.00 | 19.23 | C | |
| ATOM | 1663 | CG2 | ILE | B2099 | −2.695 | 28.629 | 27.779 | 1.00 | 18.49 | C | |
| ATOM | 1664 | CG1 | ILE | B2099 | −3.878 | 28.182 | 29.950 | 1.00 | 18.74 | C | |
| ATOM | 1665 | CD1 | ILE | B2099 | −5.199 | 27.892 | 29.299 | 1.00 | 18.23 | C | |
| ATOM | 1666 | C | ILE | B2099 | −0.997 | 30.621 | 29.221 | 1.00 | 18.65 | C | |
| ATOM | 1667 | O | ILE | B2099 | −1.570 | 31.683 | 28.973 | 1.00 | 18.86 | O | |
| ATOM | 1668 | N | TRP | B2100 | 0.273 | 30.401 | 28.907 | 1.00 | 17.78 | N | |
| ATOM | 1669 | CA | TRP | B2100 | 1.043 | 31.407 | 28.195 | 1.00 | 17.08 | C | |
| ATOM | 1670 | CB | TRP | B2100 | 2.477 | 31.487 | 28.735 | 1.00 | 15.80 | C | |
| ATOM | 1671 | CG | TRP | B2100 | 2.533 | 31.922 | 30.180 | 1.00 | 15.68 | C | |
| ATOM | 1672 | CD2 | TRP | B2100 | 2.423 | 33.264 | 30.679 | 1.00 | 15.05 | C | |
| ATOM | 1673 | CE2 | TRP | B2100 | 2.466 | 33.189 | 32.093 | 1.00 | 15.31 | C | |
| ATOM | 1674 | CE3 | TRP | B2100 | 2.289 | 34.522 | 30.070 | 1.00 | 15.15 | C | |
| ATOM | 1675 | CD1 | TRP | B2100 | 2.636 | 31.111 | 31.284 | 1.00 | 15.45 | C | |
| ATOM | 1676 | NE1 | TRP | B2100 | 2.595 | 31.868 | 32.432 | 1.00 | 15.26 | N | |
| ATOM | 1677 | CZ2 | TRP | B2100 | 2.384 | 34.328 | 32.912 | 1.00 | 15.02 | C | |
| ATOM | 1678 | CZ3 | TRP | B2100 | 2.204 | 35.658 | 30.883 | 1.00 | 15.47 | C | |
| ATOM | 1679 | CH2 | TRP | B2100 | 2.253 | 35.548 | 32.291 | 1.00 | 15.54 | C | |
| ATOM | 1680 | C | TRP | B2100 | 1.027 | 31.028 | 26.723 | 1.00 | 17.16 | C | |
| ATOM | 1681 | O | TRP | B2100 | 1.627 | 30.041 | 26.311 | 1.00 | 17.52 | O | |
| ATOM | 1682 | N | LEU | B2101 | 0.314 | 31.821 | 25.937 | 1.00 | 17.56 | N | |
| ATOM | 1683 | CA | LEU | B2101 | 0.180 | 31.575 | 24.508 | 1.00 | 17.56 | C | |
| ATOM | 1684 | CB | LEU | B2101 | −1.304 | 31.454 | 24.137 | 1.00 | 17.63 | C | |
| ATOM | 1685 | CG | LEU | B2101 | −1.641 | 31.497 | 22.635 | 1.00 | 18.34 | C | |
| ATOM | 1686 | CD1 | LEU | B2101 | −1.241 | 30.157 | 21.964 | 1.00 | 17.98 | C | |
| ATOM | 1687 | CD2 | LEU | B2101 | −3.132 | 31.768 | 22.444 | 1.00 | 17.63 | C | |
| ATOM | 1688 | C | LEU | B2101 | 0.789 | 32.685 | 23.681 | 1.00 | 17.40 | C | |
| ATOM | 1689 | O | LEU | B2101 | 0.770 | 33.849 | 24.075 | 1.00 | 17.23 | O | |
| ATOM | 1690 | N | SER | B2102 | 1.332 | 32.312 | 22.529 | 1.00 | 17.67 | N | |
| ATOM | 1691 | CA | SER | B2102 | 1.892 | 33.274 | 21.602 | 1.00 | 17.46 | C | |
| ATOM | 1692 | CB | SER | B2102 | 3.366 | 33.551 | 21.892 | 1.00 | 17.36 | C | |
| ATOM | 1693 | OG | SER | B2102 | 3.797 | 34.687 | 21.150 | 1.00 | 17.27 | O | |
| ATOM | 1694 | C | SER | B2102 | 1.731 | 32.739 | 20.185 | 1.00 | 17.83 | C | |
| ATOM | 1695 | O | SER | B2102 | 1.984 | 31.563 | 19.919 | 1.00 | 17.13 | O | |
| ATOM | 1696 | N | ILE | B2103 | 1.286 | 33.616 | 19.288 | 1.00 | 18.64 | N | |
| ATOM | 1697 | CA | ILE | B2103 | 1.084 | 33.270 | 17.888 | 1.00 | 19.37 | C | |
| ATOM | 1698 | CB | ILE | B2103 | −0.429 | 33.275 | 17.515 | 1.00 | 19.72 | C | |
| ATOM | 1699 | CG2 | ILE | B2103 | −0.625 | 32.778 | 16.079 | 1.00 | 19.83 | C | |
| ATOM | 1700 | CG1 | ILE | B2103 | −1.213 | 32.382 | 18.480 | 1.00 | 20.18 | C | |
| ATOM | 1701 | CD1 | ILE | B2103 | −2.722 | 32.470 | 18.314 | 1.00 | 20.28 | C | |
| ATOM | 1702 | C | ILE | B2103 | 1.813 | 34.299 | 17.014 | 1.00 | 19.77 | C | |
| ATOM | 1703 | O | ILE | B2103 | 1.857 | 35.489 | 17.330 | 1.00 | 18.81 | O | |
| ATOM | 1704 | N | SER | B2104 | 2.396 | 33.818 | 15.925 | 1.00 | 20.35 | N | |
| ATOM | 1705 | CA | SER | B2104 | 3.096 | 34.673 | 14.974 | 1.00 | 21.48 | C | |
| ATOM | 1706 | CB | SER | B2104 | 4.603 | 34.675 | 15.268 | 1.00 | 21.45 | C | |
| ATOM | 1707 | OG | SER | B2104 | 5.328 | 35.484 | 14.351 | 1.00 | 21.11 | O | |
| ATOM | 1708 | C | SER | B2104 | 2.826 | 34.069 | 13.598 | 1.00 | 22.45 | C | |
| ATOM | 1709 | O | SER | B2104 | 2.492 | 32.884 | 13.488 | 1.00 | 22.26 | O | |
| ATOM | 1710 | N | HIS | B2105 | 2.949 | 34.872 | 12.549 | 1.00 | 23.91 | N | |
| ATOM | 1711 | CA | HIS | B2105 | 2.725 | 34.348 | 11.207 | 1.00 | 25.44 | C | |
| ATOM | 1712 | CB | HIS | B2105 | 1.235 | 34.372 | 10.867 | 1.00 | 26.52 | C | |
| ATOM | 1713 | CG | HIS | B2105 | 0.682 | 35.754 | 10.693 | 1.00 | 27.10 | C | |
| ATOM | 1714 | CD2 | HIS | B2105 | 0.353 | 36.451 | 9.577 | 1.00 | 27.93 | C | |
| ATOM | 1715 | ND1 | HIS | B2105 | 0.427 | 36.594 | 11.755 | 1.00 | 27.73 | N | |
| ATOM | 1716 | CE1 | HIS | B2105 | −0.036 | 37.747 | 11.302 | 1.00 | 27.61 | C | |
| ATOM | 1717 | NE2 | HIS | B2105 | −0.091 | 37.686 | 9.984 | 1.00 | 27.60 | N | |
| ATOM | 1718 | C | HIS | B2105 | 3.467 | 35.117 | 10.123 | 1.00 | 25.84 | C | |
| ATOM | 1719 | O | HIS | B2105 | 3.836 | 36.274 | 10.303 | 1.00 | 25.76 | O | |
| ATOM | 1720 | N | THR | B2106 | 3.688 | 34.444 | 9.001 | 1.00 | 26.43 | N | |
| ATOM | 1721 | CA | THR | B2106 | 4.314 | 35.051 | 7.834 | 1.00 | 27.12 | C | |
| ATOM | 1722 | CB | THR | B2106 | 5.546 | 34.261 | 7.341 | 1.00 | 27.09 | C | |
| ATOM | 1723 | OG1 | THR | B2106 | 5.137 | 32.968 | 6.888 | 1.00 | 26.88 | O | |
| ATOM | 1724 | CG2 | THR | B2106 | 6.576 | 34.120 | 8.452 | 1.00 | 27.18 | C | |
| ATOM | 1725 | C | THR | B2106 | 3.213 | 34.953 | 6.777 | 1.00 | 27.59 | C | |
| ATOM | 1726 | O | THR | B2106 | 2.059 | 34.688 | 7.116 | 1.00 | 28.11 | O | |
| ATOM | 1727 | N | ASP | B2107 | 3.553 | 35.148 | 5.508 | 1.00 | 27.85 | N | |
| ATOM | 1728 | CA | ASP | B2107 | 2.554 | 35.068 | 4.449 | 1.00 | 28.22 | C | |
| ATOM | 1729 | CB | ASP | B2107 | 3.085 | 35.722 | 3.170 | 0.05 | 28.14 | C | |
| ATOM | 1730 | CG | ASP | B2107 | 3.275 | 37.220 | 3.316 | 0.05 | 28.09 | C | |
| ATOM | 1731 | OD1 | ASP | B2107 | 2.276 | 37.926 | 3.569 | 0.05 | 28.04 | O | |
| ATOM | 1732 | OD2 | ASP | B2107 | 4.422 | 37.693 | 3.177 | 0.05 | 28.08 | O | |
| ATOM | 1733 | C | ASP | B2107 | 2.116 | 33.632 | 4.138 | 1.00 | 28.63 | C | |
| ATOM | 1734 | O | ASP | B2107 | 0.995 | 33.408 | 3.684 | 1.00 | 28.77 | O | |
| ATOM | 1735 | N | GLN | B2108 | 2.988 | 32.662 | 4.392 | 1.00 | 28.69 | N | |
| ATOM | 1736 | CA | GLN | B2108 | 2.663 | 31.272 | 4.093 | 1.00 | 29.21 | C | |
| ATOM | 1737 | CB | GLN | B2108 | 3.753 | 30.666 | 3.201 | 1.00 | 30.96 | C | |
| ATOM | 1738 | CG | GLN | B2108 | 4.217 | 31.565 | 2.059 | 1.00 | 32.72 | C | |
| ATOM | 1739 | CD | GLN | B2108 | 5.386 | 30.967 | 1.282 | 1.00 | 34.31 | C | |
| ATOM | 1740 | OE1 | GLN | B2108 | 5.195 | 30.201 | 0.328 | 1.00 | 34.84 | O | |
| ATOM | 1741 | NE2 | GLN | B2108 | 6.605 | 31.306 | 1.699 | 1.00 | 34.53 | N | |
| ATOM | 1742 | C | GLN | B2108 | 2.482 | 30.366 | 5.308 | 1.00 | 28.68 | C | |
| ATOM | 1743 | O | GLN | B2108 | 1.819 | 29.337 | 5.215 | 1.00 | 28.89 | O | |
| ATOM | 1744 | N | PHE | B2109 | 3.068 | 30.741 | 6.441 | 1.00 | 27.66 | N | |
| ATOM | 1745 | CA | PHE | B2109 | 2.985 | 29.916 | 7.641 | 1.00 | 26.50 | C | |
| ATOM | 1746 | CB | PHE | B2109 | 4.347 | 29.287 | 7.953 | 1.00 | 26.61 | C | |
| ATOM | 1747 | CG | PHE | B2109 | 4.860 | 28.387 | 6.875 | 1.00 | 27.11 | C | |
| ATOM | 1748 | CD1 | PHE | B2109 | 5.577 | 28.907 | 5.799 | 1.00 | 27.30 | C | |
| ATOM | 1749 | CD2 | PHE | B2109 | 4.599 | 27.020 | 6.913 | 1.00 | 26.71 | C | |
| ATOM | 1750 | CE1 | PHE | B2109 | 6.026 | 28.073 | 4.773 | 1.00 | 27.50 | C | |
| ATOM | 1751 | CE2 | PHE | B2109 | 5.045 | 26.180 | 5.893 | 1.00 | 27.16 | C | |
| ATOM | 1752 | CZ | PHE | B2109 | 5.757 | 26.706 | 4.823 | 1.00 | 27.50 | C | |
| ATOM | 1753 | C | PHE | B2109 | 2.524 | 30.648 | 8.883 | 1.00 | 25.59 | C | |
| ATOM | 1754 | O | PHE | B2109 | 2.625 | 31.872 | 8.967 | 1.00 | 25.67 | O | |
| ATOM | 1755 | N | VAL | B2110 | 2.016 | 29.881 | 9.843 | 1.00 | 24.04 | N | |
| ATOM | 1756 | CA | VAL | B2110 | 1.580 | 30.435 | 11.114 | 1.00 | 23.42 | C | |
| ATOM | 1757 | CB | VAL | B2110 | 0.045 | 30.435 | 11.267 | 1.00 | 23.14 | C | |
| ATOM | 1758 | CG1 | VAL | B2110 | −0.498 | 29.016 | 11.276 | 1.00 | 23.49 | C | |
| ATOM | 1759 | CG2 | VAL | B2110 | −0.327 | 31.151 | 12.535 | 1.00 | 23.40 | C | |
| ATOM | 1760 | C | VAL | B2110 | 2.201 | 29.562 | 12.188 | 1.00 | 22.98 | C | |
| ATOM | 1761 | O | VAL | B2110 | 2.371 | 28.364 | 11.996 | 1.00 | 23.38 | O | |
| ATOM | 1762 | N | THR | B2111 | 2.555 | 30.161 | 13.314 | 1.00 | 22.66 | N | |
| ATOM | 1763 | CA | THR | B2111 | 3.175 | 29.402 | 14.390 | 1.00 | 22.51 | C | |
| ATOM | 1764 | CB | THR | B2111 | 4.674 | 29.721 | 14.459 | 1.00 | 23.02 | C | |
| ATOM | 1765 | OG1 | THR | B2111 | 5.310 | 28.830 | 15.373 | 1.00 | 25.57 | O | |
| ATOM | 1766 | CG2 | THR | B2111 | 4.891 | 31.148 | 14.928 | 1.00 | 23.27 | C | |
| ATOM | 1767 | C | THR | B2111 | 2.531 | 29.714 | 15.735 | 1.00 | 21.17 | C | |
| ATOM | 1768 | O | THR | B2111 | 2.013 | 30.807 | 15.939 | 1.00 | 21.65 | O | |
| ATOM | 1769 | N | ALA | B2112 | 2.565 | 28.748 | 16.649 | 1.00 | 19.97 | N | |
| ATOM | 1770 | CA | ALA | B2112 | 1.986 | 28.928 | 17.985 | 1.00 | 18.49 | C | |
| ATOM | 1771 | CB | ALA | B2112 | 0.549 | 28.422 | 18.009 | 1.00 | 16.81 | C | |
| ATOM | 1772 | C | ALA | B2112 | 2.805 | 28.227 | 19.073 | 1.00 | 17.74 | C | |
| ATOM | 1773 | O | ALA | B2112 | 3.375 | 27.168 | 18.857 | 1.00 | 16.72 | O | |
| ATOM | 1774 | N | SER | B2113 | 2.860 | 28.834 | 20.251 | 1.00 | 17.84 | N | |
| ATOM | 1775 | CA | SER | B2213 | 3.597 | 28.250 | 21.365 | 1.00 | 17.93 | C | |
| ATOM | 1776 | CB | SER | B2113 | 4.985 | 28.893 | 21.495 | 1.00 | 17.90 | C | |
| ATOM | 1777 | OG | SER | B2113 | 5.742 | 28.263 | 22.512 | 1.00 | 17.60 | O | |
| ATOM | 1778 | C | SER | B2113 | 2.805 | 28.439 | 22.653 | 1.00 | 17.35 | C | |
| ATOM | 1779 | O | SER | B2113 | 2.262 | 29.510 | 22.905 | 1.00 | 17.22 | O | |
| ATOM | 1780 | N | VAL | B2114 | 2.749 | 27.382 | 23.452 | 1.00 | 16.89 | N | |
| ATOM | 1781 | CA | VAL | B2114 | 2.020 | 27.383 | 24.708 | 1.00 | 16.75 | C | |
| ATOM | 1782 | CB | VAL | B2114 | 0.718 | 26.533 | 24.612 | 1.00 | 16.63 | C | |
| ATOM | 1783 | CG1 | VAL | B2114 | 0.112 | 26.347 | 25.999 | 1.00 | 16.16 | C | |
| ATOM | 1784 | CG2 | VAL | B2114 | −0.298 | 27.205 | 23.675 | 1.00 | 16.25 | C | |
| ATOM | 1785 | C | VAL | B2114 | 2.862 | 26.799 | 25.835 | 1.00 | 17.45 | C | |
| ATOM | 1786 | O | VAL | B2114 | 3.503 | 25.750 | 25.680 | 1.00 | 16.99 | O | |
| ATOM | 1787 | N | ILE | B2115 | 2.863 | 27.497 | 26.966 | 1.00 | 17.80 | N | |
| ATOM | 1788 | CA | ILE | B2115 | 3.566 | 27.037 | 28.156 | 1.00 | 18.72 | C | |
| ATOM | 1789 | CB | ILE | B2115 | 4.689 | 28.008 | 28.602 | 1.00 | 18.37 | C | |
| ATOM | 1790 | CG2 | ILE | B2115 | 5.385 | 27.452 | 29.846 | 1.00 | 18.27 | C | |
| ATOM | 1791 | CG1 | ILE | B2115 | 5.708 | 28.205 | 27.480 | 1.00 | 18.40 | C | |
| ATOM | 1792 | CD1 | ILE | B2115 | 6.745 | 29.289 | 27.786 | 1.00 | 17.55 | C | |
| ATOM | 1793 | C | ILE | B2115 | 2.515 | 27.000 | 29.260 | 1.00 | 19.25 | C | |
| ATOM | 1794 | O | ILE | B2115 | 1.858 | 28.004 | 29.524 | 1.00 | 19.38 | O | |
| ATOM | 1795 | N | LEU | B2116 | 2.336 | 25.846 | 29.884 | 1.00 | 20.55 | N | |
| ATOM | 1796 | CA | LEU | B2116 | 1.374 | 25.735 | 30.968 | 1.00 | 22.30 | C | |
| ATOM | 1797 | CB | LEU | B2116 | 0.628 | 24.404 | 30.902 | 1.00 | 21.86 | C | |
| ATOM | 1798 | CG | LEU | B2116 | −0.147 | 24.159 | 29.603 | 1.00 | 22.00 | C | |
| ATOM | 1799 | CD1 | LEU | B2116 | −0.976 | 22.893 | 29.732 | 1.00 | 21.49 | C | |
| ATOM | 1800 | CD2 | LEU | B2116 | −1.050 | 25.359 | 29.303 | 1.00 | 21.93 | C | |
| ATOM | 1801 | C | LEU | B2116 | 2.123 | 25.864 | 32.290 | 1.00 | 24.11 | C | |
| ATOM | 1802 | O | LEU | B2116 | 3.209 | 25.299 | 32.472 | 1.00 | 23.89 | O | |
| ATOM | 1803 | N | GLU | B2117 | 1.544 | 26.621 | 33.210 | 1.00 | 26.00 | N | |
| ATOM | 1804 | CA | GLU | B2117 | 2.177 | 26.840 | 34.493 | 1.00 | 28.60 | C | |
| ATOM | 1805 | CB | GLU | B2117 | 2.746 | 28.258 | 34.531 | 1.00 | 28.59 | C | |
| ATOM | 1806 | CG | GLU | B2117 | 3.418 | 28.627 | 35.829 | 1.00 | 30.48 | C | |
| ATOM | 1807 | CD | GLU | B2117 | 4.079 | 29.997 | 35.771 | 1.00 | 31.11 | C | |
| ATOM | 1808 | OE1 | GLU | B2117 | 3.395 | 30.972 | 35.385 | 1.00 | 31.07 | O | |
| ATOM | 1809 | OE2 | GLU | B2117 | 5.281 | 30.092 | 36.115 | 1.00 | 31.14 | O | |
| ATOM | 1810 | C | GLU | B2117 | 1.217 | 26.601 | 35.659 | 1.00 | 30.07 | C | |
| ATOM | 1811 | O | GLU | B2117 | 0.029 | 26.938 | 35.595 | 1.00 | 29.34 | O | |
| ATOM | 1812 | N | GLU | B2118 | 1.742 | 26.006 | 36.726 | 1.00 | 32.11 | N | |
| ATOM | 1813 | CA | GLU | B2118 | 0.926 | 25.712 | 37.889 | 1.00 | 34.40 | C | |
| ATOM | 1814 | CB | GLU | B2118 | 0.997 | 24.223 | 38.212 | 1.00 | 35.62 | C | |
| ATOM | 1815 | CG | GLU | B2118 | −0.294 | 23.679 | 38.772 | 1.00 | 37.98 | C | |
| ATOM | 1816 | CD | GLU | B2118 | −1.458 | 23.905 | 37.825 | 1.00 | 39.13 | C | |
| ATOM | 1817 | OE1 | GLU | B2118 | −1.403 | 23.398 | 36.682 | 1.00 | 39.94 | O | |
| ATOM | 1818 | OE2 | GLU | B2118 | −2.422 | 24.596 | 38.222 | 1.00 | 40.32 | O | |
| ATOM | 1819 | C | GLU | B2118 | 1.387 26.515 | 39.090 | 1.00 | 35.21 | C | | |
| ATOM | 1820 | O | GLU | B2118 | 2.611 | 26.552 | 39.343 | 1.00 | 35.67 | O | |
| ATOM | 1821 | OXT | GLU | B2118 | 0.507 | 27.088 | 39.767 | 1.00 | 36.45 | O | |
| TER | 1822 | | GLU | B2118 | | | | | | | |
| ATOM | 1823 | CB | MET | C3003 | 14.254 | 28.191 | 38.580 | 1.00 | 31.28 | C | |
| ATOM | 1824 | CG | MET | C3003 | 15.448 | 27.262 | 38.618 | 1.00 | 32.93 | C | |
| ATOM | 1825 | SD | MET | C3003 | 16.870 | 27.987 | 39.434 | 1.00 | 35.68 | S | |
| ATOM | 1826 | CE | MET | C3003 | 16.438 | 27.672 | 41.140 | 1.00 | 34.95 | C | |
| ATOM | 1827 | C | MET | C3003 | 13.519 | 27.087 | 36.482 | 1.00 | 29.25 | C | |
| ATOM | 1828 | O | MET | C3003 | 13.901 | 25.920 | 36.332 | 1.00 | 28.76 | O | |
| ATOM | 1829 | N | MET | C3003 | 12.435 | 26.519 | 38.666 | 1.00 | 30.53 | N | |
| ATOM | 1830 | CA | MET | C3003 | 13.052 | 27.596 | 37.845 | 1.00 | 30.20 | C | |
| ATOM | 1831 | N | ILE | C3004 | 13.491 | 27.976 | 35.493 | 1.00 | 28.16 | N | |
| ATOM | 1832 | CA | ILE | C3004 | 13.945 | 27.636 | 34.149 | 1.00 | 27.31 | C | |
| ATOM | 1833 | CB | ILE | C3004 | 13.377 | 28.604 | 33.084 | 1.00 | 27.05 | C | |
| ATOM | 1834 | CG2 | ILE | C3004 | 14.103 | 28.413 | 31.761 | 1.00 | 26.26 | C | |
| ATOM | 1835 | CG1 | ILE | C3004 | 11.876 | 28.372 | 32.919 | 1.00 | 26.90 | C | |
| ATOM | 1836 | CD1 | ILE | C3004 | 11.203 | 29.374 | 31.996 | 1.00 | 27.09 | C | |
| ATOM | 1837 | C | ILE | C3004 | 15.452 | 27.775 | 34.170 | 1.00 | 26.61 | C | |
| ATOM | 1838 | O | ILE | C3004 | 15.970 | 28.755 | 34.685 | 1.00 | 26.80 | O | |
| ATOM | 1839 | N | VAL | C3005 | 16.153 | 26.794 | 33.617 | 1.00 | 26.02 | N | |
| ATOM | 1840 | CA | VAL | C3005 | 17.611 | 26.822 | 33.581 | 1.00 | 25.10 | C | |
| ATOM | 1841 | CB | VAL | C3005 | 18.221 | 25.644 | 34.396 | 1.00 | 25.36 | C | |
| ATOM | 1842 | CG1 | VAL | C3005 | 17.820 | 25.766 | 35.869 | 1.00 | 25.11 | C | |
| ATOM | 1843 | CG2 | VAL | C3005 | 17.755 | 24.306 | 33.833 | 1.00 | 24.57 | C | |
| ATOM | 1844 | C | VAL | C3005 | 18.124 | 26.747 | 32.152 | 1.00 | 24.50 | C | |
| ATOM | 1845 | O | VAL | C3005 | 19.327 | 26.640 | 31.928 | 1.00 | 25.44 | O | |
| ATOM | 1846 | N | GLY | C3006 | 17.212 | 26.805 | 31.186 | 1.00 | 23.63 | N | |
| ATOM | 1847 | CA | GLY | C3006 | 17.612 | 26.736 | 29.791 | 1.00 | 22.31 | C | |
| ATOM | 1848 | C | GLY | C3006 | 16.446 | 26.762 | 28.820 | 1.00 | 21.49 | C | |
| ATOM | 1849 | O | GLY | C3006 | 15.332 | 26.348 | 29.156 | 1.00 | 21.47 | O | |
| ATOM | 1850 | N | HIS | C3007 | 16.700 | 27.254 | 27.609 | 1.00 | 20.33 | N | |
| ATOM | 1851 | CA | HIS | C3007 | 15.664 | 27.336 | 26.582 | 1.00 | 19.44 | C | |
| ATOM | 1852 | GB | HIS | C3007 | 14.806 | 28.591 | 26.785 | 1.00 | 18.31 | C | |
| ATOM | 1853 | CG | HIS | C3007 | 13.749 | 28.777 | 25.741 | 1.00 | 18.22 | C | |
| ATOM | 1854 | CD2 | HIS | C3007 | 13.411 | 29.857 | 24.997 | 1.00 | 18.17 | C | |
| ATOM | 1855 | ND1 | HIS | C3007 | 12.871 | 27.778 | 25.384 | 1.00 | 18.32 | N | |
| ATOM | 1856 | CE1 | HIS | C3007 | 12.036 | 28.233 | 24.467 | 1.00 | 18.15 | C | |
| ATOM | 1857 | NE2 | HIS | C3007 | 12.342 | 29.492 | 24.215 | 1.00 | 18.03 | N | |
| ATOM | 1858 | C | HIS | C3007 | 16.288 | 27.357 | 25.196 | 1.00 | 18.96 | C | |
| ATOM | 1859 | O | HIS | C3007 | 17.200 | 28.125 | 24.940 | 1.00 | 19.65 | O | |
| ATOM | 1860 | N | GLY | C3008 | 15.795 | 26.504 | 24.307 | 1.00 | 18.27 | N | |
| ATOM | 1861 | CA | GLY | C3008 | 16.340 | 26.468 | 22.965 | 1.00 | 17.73 | C | |
| ATOM | 1862 | C | GLY | C3008 | 15.307 | 26.061 | 21.932 | 1.00 | 17.73 | C | |
| ATOM | 1863 | O | GLY | C3008 | 14.398 | 25.274 | 22.215 | 1.00 | 16.98 | O | |
| ATOM | 1864 | N | ILE | C3009 | 15.426 | 26.620 | 20.735 | 1.00 | 17.47 | N | |
| ATOM | 1865 | CA | ILE | C3009 | 14.512 | 26.281 | 19.658 | 1.00 | 18.23 | C | |
| ATOM | 1866 | CB | ILE | C3009 | 13.483 | 27.414 | 19.340 | 1.00 | 17.95 | C | |
| ATOM | 1867 | CG2 | ILE | C3009 | 12.743 | 27.836 | 20.604 | 1.00 | 17.06 | C | |
| ATOM | 1868 | CG1 | ILE | C3009 | 14.192 | 28.608 | 18.683 | 1.00 | 17.87 | C | |
| ATOM | 1869 | CD1 | ILE | C3009 | 13.233 | 29.653 | 18.104 | 1.00 | 18.30 | C | |
| ATOM | 1870 | C | ILE | C3009 | 15.327 | 26.029 | 18.406 | 1.00 | 19.18 | C | |
| ATOM | 1871 | O | ILE | C3009 | 16.516 | 26.345 | 18.345 | 1.00 | 18.80 | O | |
| ATOM | 1872 | N | ASP | C3010 | 14.682 | 25.439 | 17.413 | 1.00 | 20.69 | N | |
| ATOM | 1873 | CA | ASP | C3010 | 15.330 | 25.188 | 16.144 | 1.00 | 22.71 | C | |
| ATOM | 1874 | CB | ASP | C3010 | 16.251 | 23.974 | 16.216 | 1.00 | 23.72 | C | |
| ATOM | 1875 | CG | ASP | C3010 | 16.949 | 23.710 | 14.899 | 1.00 | 24.96 | C | |
| ATOM | 1876 | OD1 | ASP | C3010 | 17.840 | 24.501 | 14.528 | 1.00 | 26.04 | O | |
| ATOM | 1877 | OD2 | ASP | C3010 | 16.598 | 22.724 | 14.219 | 1.00 | 26.67 | O | |
| ATOM | 1878 | C | ASP | C3010 | 14.307 | 24.963 | 15.045 | 1.00 | 23.50 | C | |
| ATOM | 1879 | O | ASP | C3010 | 13.239 | 24.407 | 15.278 | 1.00 | 23.37 | O | |
| ATOM | 1880 | N | ILE | C3011 | 14.645 | 25.428 | 13.851 | 1.00 | 24.39 | N | |
| ATOM | 1881 | CA | ILE | C3011 | 13.796 | 25.259 | 12.695 | 1.00 | 25.77 | C | |
| ATOM | 1882 | CB | ILE | C3011 | 13.178 | 26.600 | 12.239 | 1.00 | 25.68 | C | |
| ATOM | 1883 | CG2 | ILE | C3011 | 14.270 | 27.609 | 11.941 | 1.00 | 24.95 | C | |
| ATOM | 1884 | CG1 | ILE | C3011 | 12.274 | 26.364 | 11.022 | 1.00 | 25.90 | O | |
| ATOM | 1885 | CD1 | ILE | C3011 | 11.308 | 27.501 | 10.737 | 1.00 | 25.60 | O | |
| ATOM | 1886 | C | ILE | C3011 | 14.701 | 24.681 | 11.616 | 1.00 | 27.06 | O | |
| ATOM | 1887 | O | ILE | C3011 | 15.856 | 25.091 | 11.476 | 1.00 | 27.35 | O | |
| ATOM | 1888 | N | GLU | C3012 | 14.182 | 23.700 | 10.885 | 1.00 | 28.16 | N | |
| ATOM | 1889 | CA | GLU | C3012 | 14.937 | 23.048 | 9.829 | 1.00 | 29.36 | C | |
| ATOM | 1890 | CB | GLU | C3012 | 15.410 | 21.664 | 10.278 | 1.00 | 30.33 | C | |
| ATOM | 1891 | CG | GLU | C3012 | 16.347 | 21.633 | 11.467 | 1.00 | 31.58 | C | |
| ATOM | 1892 | CD | GLU | C3012 | 17.722 | 22.207 | 11.164 | 1.00 | 32.87 | C | |
| ATOM | 1893 | OE1 | GLU | C3012 | 18.082 | 22.322 | 9.965 | 1.00 | 32.65 | O | |
| ATOM | 1894 | OE2 | GLU | C3012 | 18.447 | 22.529 | 12.138 | 1.00 | 32.87 | O | |
| ATOM | 1895 | C | GLU | C3012 | 14.097 | 22.872 | 8.580 | 1.00 | 29.90 | O | |
| ATOM | 1896 | O | GLU | C3012 | 12.906 | 22.548 | 8.647 | 1.00 | 29.35 | O | |
| ATOM | 1897 | N | GLU | C3013 | 14.729 | 23.095 | 7.435 | 1.00 | 31.00 | N | |
| ATOM | 1898 | CA | GLU | C3013 | 14.067 | 22.913 | 6.153 | 1.00 | 32.07 | C | |
| ATOM | 1899 | CB | GLU | C3013 | 14.782 | 23.722 | 5.075 | 1.00 | 33.32 | C | |
| ATOM | 1900 | CG | GLU | C3013 | 14.202 | 23.564 | 3.684 | 1.00 | 35.05 | C | |
| ATOM | 1901 | CD | GLU | C3013 | 15.142 | 24.089 | 2.614 | 1.00 | 36.48 | C | |
| ATOM | 1902 | OE1 | GLU | C3013 | 15.645 | 25.228 | 2.761 | 1.00 | 36.82 | O | |
| ATOM | 1903 | OE2 | GLU | C3013 | 15.376 | 23.365 | 1.622 | 1.00 | 37.39 | O | |
| ATOM | 1904 | C | GLU | C3013 | 14.206 | 21.418 | 5.873 | 1.00 | 31.77 | C | |
| ATOM | 1905 | O | GLU | C3013 | 15.306 | 20.877 | 5.914 | 1.00 | 31.08 | O | |
| ATOM | 1906 | N | LEU | C3014 | 13.098 | 20.750 | 5.614 | 1.00 | 32.39 | N | |
| ATOM | 1907 | CA | LEU | C3014 | 13.130 | 19.318 | 5.360 | 1.00 | 33.65 | C | |
| ATOM | 1908 | CB | LEU | C3014 | 11.718 | 18.794 | 5.080 | 1.00 | 33.57 | C | |
| ATOM | 1909 | CG | LEU | C3014 | 10.813 | 18.685 | 6.313 | 1.00 | 34.16 | C | |
| ATOM | 1910 | CD1 | LEU | C3014 | 9.416 | 18.252 | 5.906 | 1.00 | 34.65 | C | |
| ATOM | 1911 | CD2 | LEU | C3014 | 11.404 | 17.680 | 7.280 | 1.00 | 34.38 | C | |
| ATOM | 1912 | C | LEU | C3014 | 14.069 | 18.946 | 4.220 | 1.00 | 34.37 | C | |
| ATOM | 1913 | O | LEU | C3014 | 14.736 | 17.913 | 4.271 | 1.00 | 34.47 | O | |
| ATOM | 1914 | N | ALA | C3015 | 14.128 | 19.801 | 3.204 | 1.00 | 35.44 | N | |
| ATOM | 1915 | CA | ALA | C3015 | 14.982 | 19.572 | 2.047 | 1.00 | 36.79 | C | |
| ATOM | 1916 | CB | ALA | C3015 | 14.749 | 20.655 | 1.011 | 1.00 | 36.53 | C | |
| ATOM | 1917 | C | ALA | C3015 | 16.457 | 19.511 | 2.418 | 1.00 | 37.84 | C | |
| ATOM | 1918 | O | ALA | C3015 | 17.215 | 18.737 | 1.831 | 1.00 | 38.53 | O | |
| ATOM | 1919 | N | SER | C3016 | 16.868 | 20.322 | 3.391 | 1.00 | 39.07 | N | |
| ATOM | 1920 | CA | SER | C3016 | 18.258 | 20.349 | 3.838 | 1.00 | 40.09 | C | |
| ATOM | 1921 | CB | SER | C3016 | 18.473 | 21.478 | 4.847 | 1.00 | 40.11 | C | |
| ATOM | 1922 | OG | SER | C3016 | 18.172 | 22.734 | 4.271 | 1.00 | 41.50 | O | |
| ATOM | 1923 | C | SER | C3016 | 18.628 | 19.026 | 4.481 | 1.00 | 40.95 | C | |
| ATOM | 1924 | O | SER | C3016 | 19.757 | 18.552 | 4.340 | 1.00 | 41.12 | O | |
| ATOM | 1925 | N | ILE | C3017 | 17.683 | 18.428 | 5.197 | 1.00 | 41.82 | N | |
| ATOM | 1926 | CA | ILE | C3017 | 17.951 | 17.158 | 5.849 | 1.00 | 43.16 | C | |
| ATOM | 1927 | CB | ILE | C3017 | 16.799 | 16.768 | 6.795 | 1.00 | 42.90 | C | |
| ATOM | 1928 | CG2 | ILE | C3017 | 17.170 | 15.527 | 7.598 | 1.00 | 42.32 | C | |
| ATOM | 1929 | CG1 | ILE | C3017 | 16.518 | 17.924 | 7.759 | 1.00 | 43.05 | C | |
| ATOM | 1930 | CD1 | ILE | C3017 | 17.709 | 18.332 | 8.597 | 1.00 | 42.53 | C | |
| ATOM | 1931 | C | ILE | C3017 | 18.165 | 16.076 | 4.789 | 1.00 | 44.24 | C | |
| ATOM | 1932 | O | ILE | C3017 | 19.033 | 15.215 | 4.941 | 1.00 | 44.09 | O | |
| ATOM | 1933 | N | GLU | C3018 | 17.383 | 16.128 | 3.713 | 1.00 | 45.44 | N | |
| ATOM | 1934 | CA | GLU | C3018 | 17.530 | 15.158 | 2.637 | 1.00 | 47.05 | C | |
| ATOM | 1935 | CB | GLU | C3018 | 16.516 | 15.411 | 1.524 | 1.00 | 47.54 | C | |
| ATOM | 1936 | CG | GLU | C3018 | 15.158 | 14.805 | 1.755 | 1.00 | 48.25 | C | |
| ATOM | 1937 | CD | GLU | C3018 | 14.383 | 14.664 | 0.463 | 1.00 | 48.87 | C | |
| ATOM | 1938 | QEl | GLU | C3018 | 14.159 | 15.686 | −0.220 | 1.00 | 49.40 | O | |
| ATOM | 1939 | 022 | GLU | C3018 | 14.005 | 13.525 | 0.126 | 1.00 | 49.49 | O | |
| ATOM | 1940 | C | GLU | C3018 | 18.929 | 15.261 | 2.048 | 1.00 | 47.90 | C | |
| ATOM | 1941 | O | GLU | C3018 | 19.696 | 14.299 | 2.083 | 1.00 | 47.96 | O | |
| ATOM | 1942 | N | SER | C3019 | 19.248 | 16.429 | 1.495 | 1.00 | 48.89 | N | |
| ATOM | 1943 | CA | SER | C3019 | 20.560 | 16.658 | 0.905 | 1.00 | 49.72 | C | |
| ATOM | 1944 | CB | SER | C3019 | 20.815 | 18.152 | 0.713 | 1.00 | 49.87 | C | |
| ATOM | 1945 | OG | SER | C3019 | 19.977 | 18.685 | −0.295 | 1.00 | 50.04 | O | |
| ATOM | 1946 | C | SER | C3019 | 21.614 | 16.078 | 1.825 | 1.00 | 50.34 | C | |
| ATOM | 1947 | O | SER | C3019 | 22.506 | 15.362 | 1.380 | 1.00 | 50.79 | O | |
| ATOM | 1948 | N | ALA | C3020 | 21.504 | 16.380 | 3.114 | 1.00 | 50.84 | N | |
| ATOM | 1949 | CA | ALA | C3020 | 22.461 | 15.860 | 4.083 | 1.00 | 51.57 | C | |
| ATOM | 1950 | CB | ALA | C3020 | 22.054 | 16.266 | 5.491 | 1.00 | 51.45 | C | |
| ATOM | 1951 | C | ALA | C3020 | 22.511 | 14.338 | 3.960 | 1.00 | 52.20 | C | |
| ATOM | 1952 | O | ALA | C3020 | 23.585 | 13.752 | 3.811 | 1.00 | 52.03 | O | |
| ATOM | 1953 | N | VAL | C3021 | 21.337 | 13.711 | 4.013 | 1.00 | 53.10 | N | |
| ATOM | 1954 | CA | VAL | C3021 | 21.212 | 12.259 | 3.897 | 1.00 | 53.96 | C | |
| ATOM | 1955 | CB | VAL | C3021 | 19.730 | 11.818 | 3.969 | 1.00 | 53.97 | C | |
| ATOM | 1956 | CG1 | VAL | C3021 | 19.609 | 10.329 | 3.671 | 1.00 | 54.14 | C | |
| ATOM | 1957 | CG2 | VAL | C3021 | 19.165 | 12.124 | 5.340 | 1.00 | 53.98 | C | |
| ATOM | 1958 | C | VAL | C3021 | 21.788 | 11.793 | 2.566 | 1.00 | 54.57 | C | |
| ATOM | 1959 | O | VAL | C3021 | 22.424 | 10.740 | 2.481 | 1.00 | 54.43 | O | |
| ATOM | 1960 | N | THR | C3022 | 21.553 | 12.591 | 1.530 | 1.00 | 55.21 | N | |
| ATOM | 1961 | CA | THR | C3022 | 22.041 | 12.293 | 0.194 | 1.00 | 55.84 | C | |
| ATOM | 1962 | CB | THR | C3022 | 21.456 | 13.283 | −0.831 | 1.00 | 55.80 | C | |
| ATOM | 1963 | OG1 | THR | C3022 | 20.034 | 13.124 | −0.878 | 1.00 | 55.98 | O | |
| ATOM | 1964 | CG2 | THR | C3022 | 22.036 | 13.036 | −2.214 | 1.00 | 55.79 | C | |
| ATOM | 1965 | C | THR | C3022 | 23.566 | 12.355 | 0.155 | 1.00 | 56.35 | C | |
| ATOM | 1966 | O | THR | C3022 | 24.213 | 11.386 | −0.230 | 1.00 | 56.58 | O | |
| ATOM | 1967 | N | ARG | C3023 | 24.135 | 13.489 | 0.557 | 1.00 | 56.81 | N | |
| ATOM | 1968 | CA | ARG | C3023 | 25.586 | 13.650 | 0.567 | 1.00 | 57.20 | C | |
| ATOM | 1969 | CB | ARG | C3023 | 25.966 | 15.015 | 1.151 | 1.00 | 57.25 | C | |
| ATOM | 1970 | CG | ARG | C3023 | 27.463 | 15.289 | 1.151 | 1.00 | 57.60 | C | |
| ATOM | 1971 | CD | ARG | C3023 | 27.782 | 16.762 | 1.388 | 1.00 | 57.64 | C | |
| ATOM | 1972 | NE | ARG | C3023 | 27.410 | 17.222 | 2.722 | 0.05 | 57.76 | N | |
| ATOM | 1973 | CZ | ARG | C3023 | 27.609 | 18.460 | 3.163 | 0.05 | 57.81 | C | |
| ATOM | 1974 | NH1 | ARG | C3023 | 28.176 | 19.364 | 2.374 | 0.05 | 57.84 | N | |
| ATOM | 1975 | NH2 | ARG | C3023 | 27.245 | 18.796 | 4.393 | 0.05 | 57−.82 | N | |
| ATOM | 1976 | C | ARG | C3023 | 26.203 | 12.522 | 1.390 | 1.00 | 57.47 | C | |
| ATOM | 1977 | O | ARG | C3023 | 26.328 | 11.402 | 0.903 | 1.00 | 57.73 | O | |
| ATOM | 1978 | N | HIS | C3024 | 26.592 | 12.806 | 2.630 | 1.00 | 57.66 | N | |
| ATOM | 1979 | CA | HIS | C3024 | 27.168 | 11.773 | 3.486 | 1.00 | 57.79 | C | |
| ATOM | 1980 | CB | HIS | C3024 | 27.968 | 12.404 | 4.627 | 0.05 | 57.91 | C | |
| ATOM | 1981 | CG | HIS | C3024 | 29.442 | 12.449 | 4.375 | 0.05 | 58.00 | C | |
| ATOM | 1982 | CG2 | HIS | C3024 | 30.301 | 13.491 | 4.278 | 0.05 | 58.04 | C | |
| ATOM | 1983 | ND1 | HIS | C3024 | 30.198 | 11.312 | 4.187 | 0.05 | 58.04 | N | |
| ATOM | 1984 | CBl | HIS | C3024 | 31.459 | 11.651 | 3.986 | 0.05 | 58.06 | C | |
| ATOM | 1985 | NE2 | HIS | C3024 | 31.549 | 12.968 | 4.036 | 0.05 | 58.07 | N | |
| ATOM | 1986 | C | HIS | C3024 | 26.069 | 10.873 | 4.054 | 1.00 | 57.96 | C | |
| ATOM | 1987 | O | HIS | C3024 | 25.413 | 11.223 | 5.038 | 1.00 | 58.10 | O | |
| ATOM | 1988 | N | GLU | C3025 | 25.874 | 9.710 | 3.435 | 1.00 | 57.92 | N | |
| ATOM | 1989 | CA | GLTJ C3025 | 24.846 | 8.773 | 3.879 | 1.00 | 57.71 | C | | |
| ATOM | 1990 | CB | GLU | C3025 | 24.824 | 7.535 | 2.976 | 1.00 | 58.27 | C | |
| ATOM | 1991 | CG | GLU | C3025 | 25.979 | 6.571 | 3.189 | 1.00 | 59.11 | C | |
| ATOM | 1992 | CD | GLU | C3025 | 25.784 | 5.259 | 2.446 | 1.00 | 59.59 | C | |
| ATOM | 1993 | OE1 | GLU | C3025 | 24.749 | 4.592 | 2.673 | 1.00 | 59.65 | O | |
| ATOM | 1994 | OE2 | GLU | C3025 | 26.665 | 4.893 | 1.637 | 1.00 | 59.98 | O | |
| ATOM | 1995 | C | GLU | C3025 | 25.029 | 8.341 | 5.336 | 1.00 | 57.11 | C | |
| ATOM | 1996 | O | GLU | C3025 | 24.089 | 7.857 | 5.974 | 1.00 | 57.33 | O | |
| ATOM | 1997 | N | GLY | C3026 | 26.238 | 8.508 | 5.861 | 1.00 | 56.15 | N | |
| ATOM | 1998 | CA | GLY | C3026 | 26.486 | 8.136 | 7.242 | 1.00 | 55.03 | C | |
| ATOM | 1999 | C | GLY | C3026 | 25.890 | 9.170 | 8.177 | 1.00 | 54.19 | C | |
| ATOM | 2000 | O | GLY | C3026 | 26.023 | 9.081 | 9.398 | 1.00 | 54.12 | O | |
| ATOM | 2001 | N | PHF | C3027 | 25.227 | 10.160 | 7.589 | 1.00 | 53.18 | N | |
| ATOM | 2002 | CA | PHE | C3027 | 24.601 | 11.238 | 8.343 | 1.00 | 52.05 | C | |
| ATOM | 2003 | CB | PHE | C3027 | 23.817 | 12.142 | 7.386 | 1.00 | 52.10 | C | |
| ATOM | 2004 | CG | PHE | C3027 | 23.129 | 13.289 | 8.059 | 1.00 | 52.03 | C | |
| ATOM | 2005 | CD1 | PHE | C3027 | 23.859 | 14.233 | 8.768 | 1.00 | 51.96 | C | |
| ATOM | 2006 | CG2 | PHE | C3027 | 21.743 | 13.427 | 7.984 | 1.00 | 52.34 | C | |
| ATOM | 2007 | CBl | PHE | C3027 | 23.221 | 15.304 | 9.398 | 1.00 | 52.41 | C | |
| ATOM | 2008 | CB2 | PHE | C3027 | 21.092 | 14.492 | 8.609 | 1.00 | 52.33 | C | |
| ATOM | 2009 | CZ | PHE | C3027 | 21.833 | 15.434 | 9.319 | 1.00 | 52.40 | C | |
| ATOM | 2010 | C | PHE | C3027 | 23.676 | 10.720 | 9.443 | 1.00 | 51.01 | C | |
| ATOM | 2011 | O | PHE | C3027 | 23.914 | 10.957 | 10.628 | 1.00 | 51.01 | O | |
| ATOM | 2012 | N | ALA | C3028 | 22.631 | 10.002 | 9.042 | 1.00 | 49.81 | N | |
| ATOM | 2013 | CA | ALA | C3028 | 21.648 | 9.463 | 9.979 | 1.00 | 48.74 | C | |
| ATOM | 2014 | CB | ALA | C3028 | 20.608 | 8.645 | 9.231 | 1.00 | 48.50 | C | |
| ATOM | 2015 | C | ALA | C3028 | 22.240 | 8.630 | 11.104 | 1.00 | 48.04 | C | |
| ATOM | 2016 | O | ALA | C3028 | 21.865 | 8.802 | 12.266 | 1.00 | 47.95 | O | |
| ATOM | 2017 | N | LYS | C3029 | 23.156 | 7.726 | 10.767 | 1.00 | 47.14 | N | |
| ATOM | 2018 | CA | LYS | C3029 | 23.765 | 6.874 | 11.779 | 1.00 | 46.13 | C | |
| ATOM | 2019 | CB | LYS | C3029 | 24.681 | 5.833 | 11.126 | 1.00 | 46.10 | C | |
| ATOM | 2020 | CG | LYS | C3029 | 25.918 | 6.410 | 10.460 | 0.05 | 46.12 | C | |
| ATOM | 2021 | CD | LYS | C3029 | 26.810 | 5.310 | 9.907 | 0.05 | 46.11 | C | |
| ATOM | 2022 | CB | LYS | C3029 | 28.065 | 5.884 | 9.269 | 0.05 | 46.11 | C | |
| ATOM | 2023 | NZ | LYS | C3029 | 28.873 | 6.675 | 10.238 | 0.05 | 46.11 | N | |
| ATOM | 2024 | G | LYS | C3029 | 24.548 | 7.709 | 12.789 | 1.00 | 45.54 | C | |
| ATOM | 2025 | O | LYS | C3029 | 24.739 | 7.299 | 13.936 | 1.00 | 45.54 | O | |
| ATOM | 2026 | N | ARG | C3030 | 24.997 | 8.883 | 12.361 | 1.00 | 44.50 | N | |
| ATOM | 2027 | CA | ARG | C3030 | 25.747 | 9.770 | 13.243 | 1.00 | 43.62 | C | |
| ATOM | 2028 | CB | ARG | C3030 | 26.703 | 10.650 | 12.425 | 1.00 | 43.71 | C | |
| ATOM | 2029 | CG | ARG | C3030 | 28.071 | 10.019 | 12.194 | 0.05 | 43.64 | C | |
| ATOM | 2030 | CD | ARG | C3030 | 28.410 | 9.914 | 10.717 | 0.05 | 43.63 | C | |
| ATOM | 2031 | NE | ARG | C3030 | 28.407 | 11.214 | 10.051 | 0.05 | 43.58 | N | |
| ATOM | 2032 | CZ | ARG | C3030 | 28.653 | 11.389 | 8.756 | 0.05 | 43.55 | C | |
| ATOM | 2033 | NH1 | ARG | C3030 | 28.923 | 10.347 | 7.983 | 0.05 | 43.53 | N | |
| ATOM | 2034 | NH2 | ARG | C3030 | 28.626 | 12.608 | 8.234 | 0.05 | 43.55 | N | |
| ATOM | 2035 | C | ARG | C3030 | 24.819 | 10.651 | 14.071 | 1.00 | 42.89 | C | |
| ATOM | 2036 | O | ARG | C3030 | 25.183 | 11.099 | 15.158 | 1.00 | 42.93 | O | |
| ATOM | 2037 | N | VAL | C3031 | 23.616 | 10.885 | 13.556 | 1.00 | 41.77 | N | |
| ATOM | 2038 | CA | VAL | C3031 | 22.637 | 11.724 | 14.237 | 1.00 | 40.54 | C | |
| ATOM | 2039 | CB | VAL | C3031 | 21.761 | 12.464 | 13.213 | 1.00 | 40.55 | C | |
| ATOM | 2040 | CG1 | VAL | C3031 | 20.717 | 13.298 | 13.926 | 1.00 | 40.68 | C | |
| ATOM | 2041 | CG2 | VAL | C3031 | 22.631 | 13.339 | 12.328 | 1.00 | 40.57 | C | |
| ATOM | 2042 | C | VAL | C3031 | 21.727 | 10.941 | 15.180 | 1.00 | 39.76 | C | |
| ATOM | 2043 | O | VAL | C3031 | 21.373 | 11.418 | 16.256 | 1.00 | 39.22 | O | |
| ATOM | 2044 | N | LEU | C3032 | 21.353 | 9.736 | 14.771 | 1.00 | 39.10 | N | |
| ATOM | 2045 | CA | LEU | C3032 | 20.470 | 8.905 | 15.572 | 1.00 | 38.53 | C | |
| ATOM | 2046 | CB | LEU | C3032 | 19.453 | 8.223 | 14.656 | 1.00 | 38.36 | C | |
| ATOM | 2047 | CG | LEU | C3032 | 18.612 | 9.150 | 13.767 | 1.00 | 38.42 | C | |
| ATOM | 2048 | CD1 | LEU | C3032 | 17.671 | 8.308 | 12.912 | 1.00 | 38.50 | C | |
| ATOM | 2049 | CG2 | LEU | C3032 | 17.819 | 10.131 | 14.622 | 1.00 | 37.90 | C | |
| ATOM | 2050 | C | LEU | C3032 | 21.217 | 7.848 | 16.378 | 1.00 | 38.18 | C | |
| ATOM | 2051 | O | LEU | C3032 | 22.395 | 7.585 | 16.134 | 1.00 | 38.41 | O | |
| ATOM | 2052 | N | THR | C3033 | 20.530 | 7.263 | 17.356 | 1.00 | 37.76 | N | |
| ATOM | 2053 | CA | THR | C3033 | 21.105 | 6.193 | 18.166 | 1.00 | 37.32 | C | |
| ATOM | 2054 | CB | THR | C3033 | 20.636 | 6.248 | 19.638 | 1.00 | 37.17 | C | |
| ATOM | 2055 | OCi | THR | C3033 | 19.233 | 5.967 | 19.704 | 1.00 | 37.15 | O | |
| ATOM | 2056 | CG2 | THR | C3033 | 20.900 | 7.612 | 20.236 | 1.00 | 37.54 | C | |
| ATOM | 2057 | C | THR | C3033 | 20.546 | 4.918 | 17.537 | 1.00 | 37.05 | C | |
| ATOM | 2058 | O | THR | C3033 | 19.725 | 4.992 | 16.623 | 1.00 | 36.86 | O | |
| ATOM | 2059 | N | ALA | C3034 | 20.976 | 3.759 | 18.026 | 1.00 | 37.00 | N | |
| ATOM | 2060 | CA | ALA | C3034 | 20.502 | 2.481 | 17.495 | 1.00 | 36.98 | C | |
| ATOM | 2061 | CB | ALA | C3034 | 21.121 | 1.324 | 18.279 | 1.00 | 36.85 | C | |
| ATOM | 2062 | C | ALA | C3034 | 18.978 | 2.384 | 17.538 | 1.00 | 36.93 | C | |
| ATOM | 2063 | O | ALA | C3034 | 18.331 | 2.154 | 16.516 | 1.00 | 36.95 | O | |
| ATOM | 2064 | N | LEU | C3035 | 18.401 | 2.553 | 18.721 | 1.00 | 37.04 | N | |
| ATOM | 2065 | CA | LEU | C3035 | 16.951 | 2.480 | 18.844 | 1.00 | 37.28 | C | |
| ATOM | 2066 | CB | LEU | C3035 | 16.522 | 2.844 | 20.271 | 1.00 | 37.10 | C | |
| ATOM | 2067 | CG | LEU | C3035 | 15.183 | 2.291 | 20.770 | 0.05 | 37.08 | C | |
| ATOM | 2068 | CD1 | LEU | C3035 | 14.994 | 2.675 | 22.229 | 0.05 | 37.00 | C | |
| ATOM | 2069 | CG2 | LEU | C3035 | 14.040 | 2.822 | 19.922 | 0.05 | 37.02 | C | |
| ATOM | 2070 | C | LEU | C3035 | 16.322 | 3.442 | 17.828 | 1.00 | 37.52 | C | |
| ATOM | 2071 | O | LEU | C3035 | 15.418 | 3.065 | 17.084 | 1.00 | 37.65 | O | |
| ATOM | 2072 | N | GLU | C3036 | 16.814 | 4.678 | 17.790 | 1.00 | 37.89 | N | |
| ATOM | 2073 | CA | GLU | C3036 | 16.299 | 5.679 | 16.854 | 1.00 | 38.14 | C | |
| ATOM | 2074 | CB | GLU | C3036 | 17.007 | 7.031 | 17.063 | 1.00 | 37.82 | C | |
| ATOM | 2075 | CG | GLU | C3036 | 16.486 | 7.857 | 18.244 | 1.00 | 37.71 | C | |
| ATOM | 2076 | CD | GLU | C3036 | 17.305 | 9.128 | 18.490 | 1.00 | 37.80 | C | |
| ATOM | 2077 | OEl | GLU | C3036 | 16.865 | 9.989 | 19.283 | 1.00 | 37.10 | O | |
| ATOM | 2078 | OE2 | GLU | C3036 | 18.397 | 9.265 | 17.896 | 1.00 | 37.62 | O | |
| ATOM | 2079 | C | GLU | C3036 | 16.462 | 5.232 | 15.398 | 1.00 | 38.18 | C | |
| ATOM | 2080 | O | GLU | C3036 | 15.629 | 5.551 | 14.553 | 1.00 | 38.25 | O | |
| ATOM | 2081 | N | MET | C3037 | 17.533 | 4.499 | 15.104 | 1.00 | 38.56 | N | |
| ATOM | 2082 | CA | MET | C3037 | 17.769 | 4.022 | 13.743 | 1.00 | 39.53 | C | |
| ATOM | 2083 | CB | MET | C3037 | 19.196 | 3.495 | 13.591 | 1.00 | 40.21 | C | |
| ATOM | 2084 | CG | MET | C3037 | 20.230 | 4.576 | 13.312 | 1.00 | 41.61 | C | |
| ATOM | 2085 | SD | MET | C3037 | 19.932 | 5.428 | 11.726 | 1.00 | 43.58 | S | |
| ATOM | 2086 | CB | MET | C3037 | 20.794 | 4.327 | 10.578 | 1.00 | 42.57 | C | |
| ATOM | 2087 | C | MET | C3037 | 16.782 | 2.935 | 13.337 | 1.00 | 39.68 | C | |
| ATOM | 2088 | O | MET | C3037 | 16.356 | 2.881 | 12.186 | 1.00 | 39.73 | O | |
| ATOM | 2089 | N | GLU | C3038 | 16.421 | 2.072 | 14.284 | 1.00 | 39.82 | N | |
| ATOM | 2090 | CA | GLU | C3038 | 15.473 | 0.995 | 14.012 | 1.00 | 39.88 | C | |
| ATOM | 2091 | CB | GLU | C3038 | 15.205 | 0.190 | 15.289 | 1.00 | 39.75 | C | |
| ATOM | 2092 | CG | GLU | C3038 | 16.451 | −0.428 | 15.905 | 0.05 | 39.71 | C | |
| ATOM | 2093 | CD | GLU | C3038 | 16.163 | −1.139 | 17.213 | 0.05 | 39.67 | C | |
| ATOM | 2094 | OE1 | GLU | C3038 | 15.344 | −2.083 | 17.210 | 0.05 | 39.64 | O | |
| ATOM | 2095 | OE2 | GLU | C3038 | 16.755 | −0.755 | 18.243 | 0.05 | 39.61 | O | |
| ATOM | 2096 | C | GLU | C3038 | 14.175 | 1.609 | 13.500 | 1.00 | 40.09 | C | |
| ATOM | 2097 | O | GLU | C3038 | 13.556 | 1.105 | 12.559 | 1.00 | 40.19 | O | |
| ATOM | 2098 | N | ARG | C3039 | 13.774 | 2.709 | 14.126 | 1.00 | 40.05 | N | |
| ATOM | 2099 | CA | ARG | C3039 | 12.560 | 3.418 | 13.744 | 1.00 | 40.26 | C | |
| ATOM | 2100 | CB | ARG | C3039 | 12.230 | 4.466 | 14.811 | 1.00 | 40.88 | C | |
| ATOM | 2101 | CG | ARG | C3039 | 11.106 | 5.425 | 14.468 | 1.00 | 41.43 | C | |
| ATOM | 2102 | CD | ARG | C3039 | 9.756 | 4.739 | 14.377 | 1.00 | 42.67 | C | |
| ATOM | 2103 | NE | ARG | C3039 | 8.673 | 5.723 | 14.425 | 1.00 | 43.58 | N | |
| ATOM | 2104 | CZ | ARG | C3039 | 7.394 | 5.459 | 14.169 | 1.00 | 43.85 | C | |
| ATOM | 2105 | NH1 | ARG | C3039 | 7.007 | 4.231 | 13.838 | 1.00 | 43.59 | N | |
| ATOM | 2106 | NH2 | ARG | C3039 | 6.498 | 6.435 | 14.234 | 1.00 | 44.15 | N | |
| ATOM | 2107 | C | ARG | C3039 | 12.755 | 4.090 | 12.384 | 1.00 | 39.98 | C | |
| ATOM | 2108 | O | ARG | C3039 | 11.891 | 4.009 | 11.506 | 1.00 | 39.45 | O | |
| ATOM | 2109 | N | PHE | C3040 | 13.901 | 4.745 | 12.216 | 1.00 | 40.04 | N | |
| ATOM | 2110 | CA | PHE | C3040 | 14.218 | 5.440 | 10.968 | 1.00 | 40.26 | C | |
| ATOM | 2111 | CB | PHE | C3040 | 15.603 | 6.074 | 11.061 | 1.00 | 40.27 | C | |
| ATOM | 2112 | CG | PHE | C3040 | 16.022 | 6.804 | 9.814 | 1.00 | 40.95 | C | |
| ATOM | 2113 | CD1 | PHE | C3040 | 15.431 | 8.015 | 9.466 | 1.00 | 41.11 | C | |
| ATOM | 2114 | CG2 | PHE | C3040 | 17.013 | 6.282 | 8.989 | 1.00 | 41.10 | C | |
| ATOM | 2115 | CBl | PHE | C3040 | 15.822 | 8.699 | 8.315 | 1.00 | 41.43 | C | |
| ATOM | 2116 | CB2 | PHE | C3040 | 17.412 | 6.956 | 7.835 | 1.00 | 41.44 | C | |
| ATOM | 2117 | CZ | PHE | C3040 | 16.815 | 8.169 | 7.497 | 1.00 | 41.61 | C | |
| ATOM | 2118 | C | PHE | C3040 | 14.181 | 4.507 | 9.756 | 1.00 | 40.43 | C | |
| ATOM | 2119 | O | PHE | C3040 | 13.467 | 4.757 | 8.784 | 1.00 | 39.60 | O | |
| ATOM | 2120 | N | THR | C3041 | 14.956 | 3.429 | 9.833 | 1.00 | 40.82 | N | |
| ATOM | 2121 | CA | THR | C3041 | 15.043 | 2.459 | 8.749 | 1.00 | 41.42 | C | |
| ATOM | 2122 | CB | THR | C3041 | 16.154 | 1.435 | 9.018 | 1.00 | 41.55 | C | |
| ATOM | 2123 | OG1 | THR | C3041 | 15.782 | 0.597 | 10.121 | 1.00 | 42.56 | O | |
| ATOM | 2124 | CG2 | THR | C3041 | 17.450 | 2.149 | 9.351 | 1.00 | 41.22 | C | |
| SLAYER | | | | | | | | | | | |
| ATOM | 2125 | C | THR | C3041 | 13.749 | 1.702 | 8.463 | 1.00 | 41.42 | C | |
| ATOM | 2126 | O | THR | C3041 | 13.630 | 1.072 | 7.415 | 1.00 | 41.80 | O | |
| ATOM | 2127 | N | SER | C3042 | 12.783 | 1.757 | 9.377 | 1.00 | 41.30 | N | |
| ATOM | 2128 | CA | SER | C3042 | 11.521 | 1.059 | 9.152 | 1.00 | 41.35 | C | |
| ATOM | 2129 | CB | SER | C3042 | 10.998 | 0.445 | 10.454 | 1.00 | 41.25 | C | |
| ATOM | 2130 | OG | SER | C3042 | 10.501 | 1.442 | 11.324 | 1.00 | 42.03 | O | |
| ATOM | 2131 | C | SER | C3042 | 10.469 | 1.996 | 6.569 | 1.00 | 41.30 | C | |
| ATOM | 2132 | 0 | SER | C3042 | 9.334 | 1.589 | 8.311 | 1.00 | 41.45 | O | |
| ATOM | 2133 | N | LEU | C3043 | 10.850 | 3.255 | 8.373 | 1.00 | 41.13 | N | |
| ATOM | 2134 | CA | LEU | C3043 | 9.956 | 4.262 | 7.808 | 1.00 | 40.94 | C | |
| ATOM | 2135 | CB | LEU | C3043 | 10.026 | 5.566 | 8.618 | 1.00 | 40.70 | C | |
| ATOM | 2136 | CG | LEU | C3043 | 9.382 | 5.618 | 10.012 | 1.00 | 40.56 | C | |
| ATOM | 2137 | COl | LEU | C3043 | 9.835 | 6.871 | 10.735 | 1.00 | 40.07 | C | |
| ATOM | 2138 | CG2 | LEU | C3043 | 7.859 | 5.586 | 9.891 | 1.00 | 40.06 | . | C |
| ATOM | 2139 | C | LEU | C3043 | 10.398 | 4.528 | 6.375 | 1.00 | 41.07 | C | |
| ATOM | 2140 | 0 | LEU | C3043 | 11.494 | 4.128 | 5.978 | 1.00 | 40.93 | O | |
| ATOM | 2141 | N | LYS | C3044 | 9.550 | 5.203 | 5.604 | 1.00 | 41.26 | N | |
| ATOM | 2142 | CA | LYS | C3044 | 9.871 | 5.513 | 4.214 | 1.00 | 41.42 | C | |
| ATOM | 2143 | CB | LYS | C3044 | 9.481 | 4.346 | 3.307 | 1.00 | 42.49 | C | |
| ATOM | 2144 | CG | LYS | C3044 | 1.982 | 4.199 | 3.107 | 1.00 | 43.64 | C | |
| ATOM | 2145 | CD | LYS | C3044 | 7.678 | 3.191 | 2.012 | 1.00 | 45.17 | C | |
| ATOM | 2146 | CB | LYS | C3044 | 6.206 | 3.228 | 1.616 | 1.00 | 45.99 | C | |
| ATOM | 2147 | NZ | LYS | C3044 | 5.900 | 2.249 | 0.535 | 1.00 | 47.05 | N | |
| ATOM | 2148 | G | LYS | C3044 | 9.147 | 6.768 | 3.739 | 1.00 | 40.92 | C | |
| ATOM | 2149 | 0 | LYS | C3044 | 8.191 | 7.219 | 4.365 | 1.00 | 40.90 | O | |
| ATOM | 2150 | N | GLY | C3045 | 9.605 | 7.820 | 2.621 | 1.00 | 40.22 | N | |
| ATOM | 2151 | CA | GLY | C3045 | 8.986 | 8.510 | 2.072 | 1.00 | 39.26 | C | |
| ATOM | 2152 | C | GLY | C3045 | 9.182 | 9.736 | 2.937 | 1.00 | 38.68 | C | |
| ATOM | 2153 | 0 | GLY | C3045 | 10.142 | 9.829 | 3.694 | 1.00 | 38.24 | 0 | |
| ATOM | 2154 | N | ARG | C3046 | 8.260 | 10.683 | 2.818 | 1.00 | 38.56 | N | |
| ATOM | 2155 | CA | ARG | C3046 | 8.318 | 11.921 | 3.589 | 1.00 | 38.49 | C | |
| ATOM | 2156 | CB | ARG | C3046 | 7.107 | 12.793 | 3.251 | 1.00 | 38.44 | C | |
| ATOM | 2157 | CG | ARG | C3046 | 7.104 | 14.158 | 3.912 | 0.05 | 38.46 | C | |
| ATOM | 2158 | CD | ARG | C3046 | 5.887 | 14.963 | 3.483 | 0.05 | 38.40 | C | |
| ATOM | 2159 | NE | ARG | C3046 | 5.874 | 15.200 | 2.042 | 0.05 | 38.36 | N | |
| ATOM | 2160 | CZ | ARG | C3046 | 4.887 | 15.809 | 1.393 | 0.05 | 38.32 | C | |
| ATOM | 2161 | NH1 | ARG | C3046 | 4.963 | 15.982 | 0.081 | 0.05 | 38.29 | N | |
| ATOM | 2162 | NH2 | ARG | C3046 | 3.822 | 16.241 | 2.055 | 0.05 | 38.31 | N | |
| ATOM | 2163 | C | ARG | C3046 | 8.372 | 11.647 | 5.093 | 1.00 | 38.25 | C | |
| ATOM | 2164 | 0 | ARG | C3046 | 9.027 | 12.365 | 5.830 | 1.00 | 38.13 | O | |
| ATOM | 2165 | N | ARG | C3047 | 7.696 | 10.595 | 5.538 | 1.00 | 38.32 | N | |
| ATOM | 2166 | CA | ARG | C3047 | 7.673 | 10.234 | 6.953 | 1.00 | 38.52 | C | |
| ATOM | 2167 | CB | ARG | C3047 | 6.727 | 9.051 | 7.169 | 1.00 | 39.67 | C | |
| ATOM | 2168 | CG | ARG | C3047 | 5.251 | 9.432 | 7.225 | 1.00 | 41.67 | C | |
| ATOM | 2169 | CD | ARG | C3047 | 4.369 | 8.277 | 6.767 | 1.00 | 43.70 | C | |
| ATOM | 2170 | NE | ARG | C3047 | 3.141 | 8.152 | 7.550 | 1.00 | 45.24 | N | |
| ATOM | 2171 | CZ | ARG | C3047 | 3.112 | 7.824 | 8.840 | 1.00 | 46.12 | C | |
| ATOM | 2172 | NH1 | ARG | C3047 | 4.248 | 7.589 | 9.495 | 1.00 | 46.08 | N | |
| ATOM | 2173 | NH2 | ARG | C3047 | 1.947 | 7.722 | 9.474 | 1.00 | 45.94 | N | |
| ATOM | 2174 | C | ARG | C3047 | 9.042 | 9.906 | 7.554 | 1.00 | 38.04 | C | |
| ATOM | 2175 | 0 | ARG | C3047 | 9.287 | −10.174 | 8.731 | 1.00 | 37.75 | O | |
| ATOM | 2176 | N | GLN | C3048 | 9.931 | 9.820 | 6.757 | 1.00 | 37.49 | N | |
| ATOM | 2177 | CA | GLN | C3048 | 11.258 | 8.966 | 7.251 | 1.00 | 36.91 | C | |
| ATOM | 2178 | CB | GLN | C3048 | 11.912 | 7.919 | 6.349 | 1.00 | 36.94 | C | |
| ATOM | 2179 | CG | GLN | C3048 | 13.283 | 7.496 | 6.849 | 1.00 | 37.10 | C | |
| ATOM | 2180 | CD | GLN | C3048 | 14.092 | 6.744 | 5.813 | 1.00 | 37.40 | C | |
| ATOM | 2181 | OE1 | GLN | C3048 | 14.233 | 7.196 | 4.680 | 1.00 | 37.42 | O | |
| ATOM | 2182 | NE2 | GLN | C3048 | 14.643 | 5.600 | 6.202 | 1.00 | 37.18 | N | |
| ATOM | 2183 | C | GLN | C3048 | 12.186 | 10.176 | 7.367 | 1.00 | 36.61 | C | |
| ATOM | 2184 | 0 | GLN | C3048 | 12.971 | 10.284 | 8.315 | 1.00 | 36.44 | O | |
| ATOM | 2185 | N | ILE | C3049 | 12.101 | 11.082 | 6.403 | 1.00 | 35.91 | N | |
| ATOM | 2186 | CA | ILE | U3049 | 12.932 | 12.271 | 6.429 | 1.00 | 35.80 | C | |
| ATOM | 2187 | CB | ILE | U3049 | 12.906 | 12.991 | 5.054 | 1.00 | 36.41 | C | |
| ATOM | 2188 | CG2 | ILE | U3049 | 11.482 | 13.209 | 4.607 | 1.00 | 37.11 | C | |
| ATOM | 2189 | 001 | ILE | U3049 | 13.678 | 14.307 | 5.136 | 1.00 | 36.64 | C | |
| ATOM | 2190 | CD1 | ILE | U3049 | 15.150 | 14.108 | 5.433 | 1.00 | 37.22 | C | |
| ATOM | 2191 | C | ILE | U3049 | 12.436 | 13.201 | 7.542 | 1.00 | 35.41 | C | |
| ATOM | 2192 | 0 | ILE | U3049 | 13.231 | 13.824 | 8.254 | 1.00 | 35.47 | O | |
| ATOM | 2193 | N | GLU | C3050 | 11.119 | 13.280 | 7.707 | 1.00 | 34.48 | N | |
| ATOM | 2194 | CA | GLU | C3050 | 10.546 | 14.118 | 8.756 | 1.00 | 33.55 | C | |
| ATOM | 2195 | CB | GLU | C3050 | 9.023 | 14.074 | 8.699 | 1.00 | 34.18 | C | |
| ATOM | 2196 | CO | GLU | C3050 | 8.471 | 14.609 | 7.410 | 1.00 | 36.22 | C | |
| ATOM | 2197 | CD | GLU | C3050 | 7.005 | 14.309 | 7.242 | 1.00 | 37.41 | C | |
| ATOM | 2198 | OE1 | GLU | C3050 | 6.543 | 13.291 | 7.807 | 1.00 | 38.55 | O | |
| ATOM | 2199 | OE2 | GLU | C3050 | 6.318 | 15.078 | 6.531 | 1.00 | 37.87 | O | |
| ATOM | 2200 | C | GLU | C3050 | 11.018 | 13.620 | 10.116 | 1.00 | 32.30 | C | |
| ATOM | 2201 | 0 | GLU | C3050 | 11.275 | 14.412 | 11.023 | 1.00 | 31.96 | O | |
| ATOM | 2202 | N | TYR | C3051 | 11.130 | 12.303 | 10.251 | 1.00 | 30.44 | N | |
| ATOM | 2203 | CA | TYR | C3051 | 11.574 | 11.715 | 11.501 | 1.00 | 29.38 | C | |
| ATOM | 2204 | CB | TYR | C3051 | 11.438 | 10.191 | 11.433 | 1.00 | 28.33 | C | |
| ATOM | 2205 | CG | TYR | C3051 | 11.936 | 9.478 | 12.675 | 1.00 | 27.96 | C | |
| ATOM | 2206 | CD1 | TYR | C3051 | 13.234 | 8.964 | 12.741 | 1.00 | 27.15 | C | |
| ATOM | 2207 | CE1 | TYR | C3051 | 13.712 | 8.371 | 13.904 | 1.00 | 26.96 | C | |
| ATOM | 2208 | CG2 | TYR | C3051 | 11.127 | 9.373 | 13.809 | 1.00 | 27.47 | C | |
| ATOM | 2209 | CB2 | TYR | C3051 | 11.593 | 8.788 | 14.967 | 1.00 | 27.13 | C | |
| ATOM | 2210 | CZ | TYR | 03051 | 12.885 | 8.290 | 15.014 | 1.00 | 27.21 | C | |
| ATOM | 2211 | OH | TYR | C3051 | 13.344 | 7.735 | 16.187 | 1.00 | 27.00 | O | |
| ATOM | 2212 | C | TYR | 03051 | 13.019 | 12.117 | 11.818 | 1.00 | 29.01 | C | |
| ATOM | 2213 | 0 | TYR | C3051 | 13.342 | 12.486 | 12.946 | 1.00 | 28.83 | 0 | |
| ATOM | 2214 | N | LEU | C3052 | 13.886 | 12.063 | 10.812 | 1.00 | 28.73 | N | |
| ATOM | 2215 | CA | LEU | C3052 | 15.287 | 12.417 | 11.009 | 1.00 | 28.07 | C | |
| ATOM | 2216 | CB | LEU | C3052 | 16.105 | 12.059 | 9.761 | 1.00 | 27.77 | C | |
| ATOM | 2217 | CO | LEU | C3052 | 17.603 | 12.372 | 9.816 | 1.00 | 28.06 | C | |
| ATOM | 2218 | CD1 | LEU | C3052 | 18.267 | 11.639 | 10.972 | 1.00 | 28.26 | C | |
| ATOM | 2219 | CG2 | LEU | C3052 | 18.241 | 11.957 | 8.516 | 1.00 | 28.46 | C | |
| ATOM | 2220 | C | LEU | C3052 | 15.419 | 13.909 | 11.311 | 1.00 | 27.59 | C | |
| ATOM | 2221 | O | LEU | C3052 | 16.156 | 14.309 | 12.217 | 1.00 | 26.99 | O | |
| ATOM | 2222 | N | ALA | C3053 | 14.704 | 14.727 | 10.544 | 1.00 | 27.06 | N | |
| ATOM | 2223 | CA | ALA | C3053 | 14.743 | 16.168 | 10.738 | 1.00 | 26.76 | C | |
| ATOM | 2224 | GB | ALA | 03053 | 13.898 | 16.852 | 9.691 | 1.00 | 26.56 | C | |
| ATOM | 2225 | C | ALA | 03053 | 14.223 | 16.489 | 12.134 | 1.00 | 26.71 | C | |
| ATOM | 2226 | O | ALA | C3053 | 14.802 | 17.298 | 12.854 | 1.00 | 26.43 | O | |
| ATOM | 2227 | N | GLY | C3054 | 13.130 | 15.831 | 12.510 | 1.00 | 26.77 | N | |
| ATOM | 2228 | CA | GLY | C3054 | 12.541 | 16.043 | 13.815 | 1.00 | 26.67 | C | |
| ATOM | 2229 | C | GLY | C3054 | 13.512 | 15.753 | 14.934 | 1.00 | 26.53 | C | |
| ATOM | 2230 | 0 | GLY | C3054 | 13.611 | 16.521 | 15.882 | 1.00 | 26.44 | O | |
| ATOM | 2231 | N | ARG | C3055 | 14.231 | 14.643 | 14.836 | 1.00 | 26.68 | N | |
| ATOM | 2232 | CA | ARG | C3055 | 15.198 | −14.290 | 15.870 | 1.00 | 26.82 | C | |
| ATOM | 2233 | GB | ARG | C3055 | 15.705 | 12.868 | 15.666 | 1.00 | 26.65 | C | |
| ATOM | 2234 | GO | ARG | C3055 | 14.968 | 11.843 | 16.498 | 1.00 | 26.91 | C | |
| ATOM | 2235 | CD | ARG | C3055 | 13.458 | 11.922 | 16.298 | 1.00 | 27.49 | C | |
| ATOM | 2236 | NE | ARG | C3055 | 12.759 | 11.110 | 17.292 | 1.00 | 27.69 | N | |
| ATOM | 2237 | CZ | ARG | 03055 | 11.442 | 11.086 | 17.454 | 1.00 | 27.76 | C | |
| ATOM | 2238 | NH1 | ARG | C3055 | 10.662 | 11.831 | 16.685 | 1.00 | 27.51 | N | |
| ATOM | 2239 | NH2 | ARG | 03055 | 10.906 | 10.324 | 18.398 | 1.00 | 28.13 | N | |
| ATOM | 2240 | C | ARG | C3055 | 16.360 | 15.260 | 15.869 | 1.00 | 26.96 | C | |
| ATOM | 2241 | 0 | ARG | C3055 | 16.834 | 15.667 | 16.928 | 1.00 | 26.54 | O | |
| ATOM | 2242 | N | TRP | C3056 | 16.816 | 15.631 | 14.676 | 1.00 | 27.71 | N | |
| ATOM | 2243 | CA | TRP | C3056 | 17.917 | 16.575 | 14.556 | 1.00 | 28.70 | C | |
| ATOM | 2244 | CB | TRP | C3056 | 18.247 | 16.837 | 13.082 | 1.00 | 30.14 | C | |
| ATOM | 2245 | CG | TRP | C3056 | 19.074 | 18.069 | 12.881 | 1.00 | 32.25 | C | |
| ATOM | 2246 | CG2 | TRP | C3056 | 20.495 | 18.129 | 12.703 | 1.00 | 33.03 | C | |
| ATOM | 2247 | CB2 | TRP | C3056 | 20.854 | 19.497 | 12.662 | 1.00 | 33.33 | C | |
| ATOM | 2248 | CB3 | TRP | C3056 | 21.500 | 17.163 | 12.579 | 1.00 | 33.56 | C | |
| ATOM | 2249 | COl | TRP | C3056 | 18.639 | 19.370 | 12.930 | 1.00 | 32.92 | C | |
| ATOM | 2250 | NEl | TRP | C3056 | 19.704 | 20.230 | 12.803 | 1.00 | 33.50 | N | |
| ATOM | 2251 | CZ2 | TRP | C3056 | 22.175 | 19.920 | 12.505 | 1.00 | 33.41 | C | |
| ATOM | 2252 | CZ3 | TRP | C3056 | 22.818 | 17.587 | 12.422 | 1.00 | 33.92 | C | |
| ATOM | 2253 | CH2 | TRP | C3056 | 23.141 | 18.954 | 12.367 | 1.00 | 33.82 | C | |
| ATOM | 2254 | C | TRFKC3OS6 | 17.498 | 17.875 | 15.230 | 1.00 | 28.10 | C | | |
| ATOM | 2255 | 0 | TRP | C3056 | 18.144 | 18.357 | 16.163 | 1.00 | 27.48 | O | |
| ATOM | 2256 | N | SER | C3057 | 16.397 | 18.429 | 14.749 | 1.00 | 27.65 | N | |
| ATOM | 2257 | CA | SER | C3057 | 15.868 | 19.667 | 15.288 | 1.00 | 27.54 | C | |
| ATOM | 2258 | CB | SER | C3057 | 14.513 | 19.959 | 14.654 | 1.00 | 27.86 | C | |
| ATOM | 2259 | OG | SER | C3057 | 14.233 | 21.344 | 14.718 | 1.00 | 29.93 | O | |
| ATOM | 2260 | C | SER | C3057 | 15.731 | 19.622 | 16.812 | 1.00 | 26.85 | C | |
| ATOM | 2261 | 0 | SER | C3057 | 16.059 | 20.588 | 17.503 | 1.00 | 27.24 | O | |
| ATOM | 2262 | N | ALA | C3058 | 15.257 | 18.501 | 17.340 | 1.00 | 25.77 | N | |
| ATOM | 2263 | CA | ALA | C3058 | 15.080 | 18.369 | 18.780 | 1.00 | 25.50 | C | |
| ATOM | 2264 | CB | ALA | C3058 | 14.334 | 17.077 | 19.094 | 1.00 | 25.02 | C | |
| ATOM | 2265 | C | ALA | C3058 | 16.411 | 18.409 | 19.546 | 1.00 | 25.26 | C | |
| ATOM | 2266 | 0 | ALA | C3058 | 16.513 | 19.024 | 20.607 | 1.00 | 24.25 | O | |
| ATOM | 2267 | N | LYS | C3059 | 17.430 | 17.752 | 19.000 | 1.00 | 25.32 | N | |
| ATOM | 2268 | CA | LYS | C3059 | 18.736 | 17.704 | 19.646 | 1.00 | 25.14 | C | |
| ATOM | 2269 | CB | LYS | C3059 | 19.568 | 16.577 | 19.035 | 1.00 | 24.87 | C | |
| ATOM | 2270 | CG | LYS | C3059 | 18.928 | 15.218 | 19.254 | 1.00 | 24.63 | C | |
| ATOM | 2271 | CD | LYS | C3059 | 19.667 | 14.091 | 18.570 | 1.00 | 24.80 | C | |
| ATOM | 2272 | CB | LYS | C3059 | 18.998 | 12.746 | 18.869 | 1.00 | 24.92 | C | |
| ATOM | 2273 | NZ | LYS | C3059 | 19.674 | 11.614 | 18.185 | 1.00 | 24.83 | N | |
| ATOM | 2274 | G | LYS | C3059 | 19.466 | 19.033 | 19.547 | 1.00 | 25.56 | C | |
| ATOM | 2275 | 0 | LYS | C3059 | 20.262 | 19.388 | 20.415 | 1.00 | 25.33 | O | |
| ATOM | 2276 | N | GLU | C3060 | 19.183 | 19.784 | 18.493 | 1.00 | 26.00 | N | |
| ATOM | 2277 | CA | GLU | C3060 | 19.818 | 21.076 | 18.326 | 1.00 | 26.76 | C | |
| ATOM | 2278 | CB | GLU | C3060 | 19.568 | 21.618 | 16.919 | 1.00 | 28.10 | C | |
| ATOM | 2279 | CG | GLU | C3060 | 20.545 | 22.717 | 16.530 | 1.00 | 31.38 | C | |
| ATOM | 2280 | CD | GLU | C3060 | 22.005 | 22.278 | 16.691 | 1.00 | 33.19 | C | |
| ATOM | 2281 | OEI | GLU | C3060 | 22.444 | 21.369 | 15.947 | 1.00 | 33.60 | O | |
| ATOM | 2282 | OE2 | GLU | C3060 | 22.709 | 22.839 | 17.569 | 1.00 | 34.17 | O | |
| ATOM | 2283 | C | GLU | C3060 | 19.243 | 22.023 | 19.377 | 1.00 | 26.31 | C | |
| ATOM | 2284 | 0 | GLU | C3060 | 19.985 | 22.698 | 20.089 | 1.00 | 25.94 | O | |
| ATOM | 2285 | N | ALA | C3061 | 17.914 | 22.048 | 19.477 | 1.00 | 25.95 | N | |
| ATOM | 2286 | CA | ALA | C3061 | 17.221 | 22.899 | 20.442 | 1.00 | 25.01 | C | |
| ATOM | 2287 | CB | ALA | C3061 | 15.712 | 22.700 | 20.342 | 1.00 | 24.05 | C | |
| ATOM | 2288 | C | ALA | C3061 | 17.696 | 22.562 | 21.846 | 1.00 | 24.62 | C | |
| ATOM | 2289 | 0 | ALA | C3061 | 17.925 | 23.457 | 22.663 | 1.00 | 24.28 | O | |
| ATOM | 2290 | N | PE−LE C3062 | 17.837 | 21.271 | 22.129 | 1.00 | 24.17 | N | | |
| ATOM | 2291 | CA | PHE | C3062 | 18.301 | 20.850 | 23.440 | 1.00 | 24.63 | C | |
| ATOM | 2292 | CB | PHE | C3062 | 18.224 | 19.321 | 23.605 | 1.00 | 24.68 | C | |
| ATOM | 2293 | CG | PHE | C3062 | 18.875 | 18.828 | 24.871 | 1.00 | 24.04 | C | |
| ATOM | 2294 | CD1 | PHE | C3062 | 20.238 | 18.538 | 24.900 | 1.00 | 24.34 | C | |
| ATOM | 2295 | CG2 | PHE | C3062 | 18.152 | 18.756 | 26.053 | 1.00 | 23.91 | C | |
| ATOM | 2296 | CBl | PHE | C3062 | 20.872 | 18.189 | 26.093 | 1.00 | 24.30 | C | |
| ATOM | 2297 | CB2 | PHE | C3062 | 18.774 | 18.411 | 27.255 | 1.00 | 24.14 | C | |
| ATOM | 2298 | CZ | PHE | C3062 | 20.139 | 18.128 | 27.274 | 1.00 | 24.07 | C | |
| ATOM | 2299 | C | PHE | C3062 | 19.732 | 21.316 | 23.701 | 1.00 | 24.85 | C | |
| ATOM | 2300 | 0 | PHE | C3062 | 20.054 | 21.744 | 24.804 | .1.00 | 24.51 | O | |
| ATOM | 2301 | N | SER | C3063 | 20.589 | 21.219 | 22.689 | 1.00 | 25.44 | N | |
| ATOM | 2302 | CA | SER | C3063 | 21.976 | 21.650 | 22.933 | 1.00 | 26.48 | C | |
| ATOM | 2303 | CB | SER | C3063 | 22.777 | 21.312 | 21.576 | 1.00 | 26.81 | C | |
| ATOM | 2304 | OG | SER | C3063 | 22.872 | 19.910 | 21.403 | 1.00 | 28.20 | O | |
| ATOM | 2305 | C | SER | C3063 | 22.059 | 23.149 | 23.082 | 1.00 | 26.77 | C | |
| ATOM | 2306 | 0 | SER | C3063 | 22.891 | 23.614 | 23.859 | 1.00 | 26.78 | O | |
| ATOM | 2307 | N | LYS | C3064 | 21.194 | 23.902 | 22.416 | 1.00 | 26.78 | N | |
| ATOM | 2308 | CA | LYS | C3064 | 21.188 | 25.342 | 22.565 | 1.00 | 27.25 | C | |
| ATOM | 2309 | CB | LYS | C3064 | 20.379 | 25.973 | 21.431 | 1.00 | 26.90 | C | |
| ATOM | 2310 | CG | LYS | C3064 | 20.971 | 25.696 | 20.059 | 1.00 | 26.47 | C | |
| ATOM | 2311 | CD | LYS | C3064 | 20.066 | 26.159 | 18.938 | 1.00 | 26.20 | C | |
| ATOM | 2312 | CB | LYS | C3064 | 19.799 | 27.664 | 18.988 | 1.00 | 26.50 | C | |
| ATOM | 2313 | NZ | LYS | C3064 | 18.996 | 28.088 | 17.806 | 1.00 | 26.49 | N | |
| ATOM | 2314 | G | LYS | C3064 | 20.616 | 25.718 | 23.923 | 1.00 | 28.08 | C | |
| ATOM | 2315 | 0 | LYS | C3064 | 20.987 | 26.738 | 24.500 | 1.00 | 27.79 | O | |
| ATOM | 2316 | N | ALA | C3065 | 19.717 | 24.882 | 24.433 | 1.00 | 29.11 | N | |
| ATOM | 2317 | CA | ALA | C3065 | 19.111 | 25.124 | 25.735 | 1.00 | 30.40 | C | |
| ATOM | 2318 | CB | ALA | C3065 | 17.957 | 24.163 | 25.968 | 1.00 | 29.37 | C | |
| ATOM | 2319 | C | ALA | C3065 | 20.179 | 24.932 | 26.804 | 1.00 | 31.79 | C | |
| ATOM | 2320 | O | ALA | C3065 | 20.228 | 25.668 | 27.786 | 1.00 | 31.85 | O | |
| ATOM | 2321 | N | MET | C3066 | 21.035 | 23.939 | 26.595 | 1.00 | 33.85 | N | |
| ATOM | 2322 | CA | MET | C3066 | 22.114 | 23.628 | 27.524 | 1.00 | 36.16 | C | |
| ATOM | 2323 | CB | MET | C3066 | 22.666 | 22.232 | 27.242 | 1.00 | 36.40 | C | |
| ATOM | 2324 | CG | MET | C3066 | 21.804 | 21.097 | 27.752 | 1.00 | 37.31 | C | |
| ATOM | 2325 | SD | MET | C3066 | 21.824 | 20.993 | 29.551 | 1.00 | 38.61 | S | |
| ATOM | 2326 | CB | MET | C3066 | 23.502 | 20.468 | 29.811 | 1.00 | 38.12 | C | |
| ATOM | 2327 | C | MET | C3066 | 23.247 | 24.638 | 27.413 | 1.00 | 37.57 | C | |
| ATOM | 2328 | 0 | MET | C3066 | 23.958 | 24.893 | 28.379 | 1.00 | 37.83 | O | |
| ATOM | 2329 | N | GLY | C3067 | 23.411 | 25.209 | 26.226 | 1.00 | 39.17 | N | |
| ATOM | 2330 | CA | GLY | C3067 | 24.470 | 26.177 | 26.024 | 1.00 | 41.14 | C | |
| ATOM | 2331 | C | GLY | C3067 | 25.772 | 25.475 | 25.697 | 1.00 | 42.59 | C | |
| ATOM | 2332 | 0 | GLY | C3067 | 26.831 | 25.822 | 26.226 | 1.00 | 42.74 | O | |
| ATOM | 2333 | N | THR | C3068 | 25.687 | 24.476 | 24.825 | 1.00 | 43.64 | N | |
| ATOM | 2334 | CA | THR | C3068 | 26.853 | 23.710 | 24.418 | 1.00 | 44.94 | C | |
| ATOM | 2335 | CB | THR | C3068 | 26.723 | 22.243 | 24.877 | 1.00 | 45.62 | C | |
| ATOM | 2336 | OGi | THR | C3068 | 27.927 | 21.529 | 24.559 | 1.00 | 46.68 | O | |
| ATOM | 2337 | CG2 | THR | C3068 | 25.539 | 21.572 | 24.188 | 1.00 | 45.83 | C | |
| ATOM | 2338 | C | THR | C3068 | 26.994 | 23.755 | 22.898 | 1.00 | 45.32 | C | |
| ATOM | 2339 | 0 | THR | C3068 | 26.027 | 24.200 | 22.242 | 1.00 | 45.93 | O | |
| ATOM | 2340 | OXT | THR | C3068 | 28.057 | 23.348 | 22.377 | 1.00 | 45.68 | O | |
| TER | 2341 | THR | | C3068 | | | | | | | |
| ATOM | 2342 | C | GLY | C3074 | 25.582 | 13.730 | 21.033 | 1.00 | 50.05 | C | |
| ATOM | 2343 | 0 | GLY | C3074 | 24.831 | 13.333 | 21.925 | 1.00 | 50.04 | O | |
| ATOM | 2344 | N | GLY | C3074 | 27.008 | 15.502 | 22.091 | 1.00 | 50.49 | N | |
| ATOM | 2345 | CA | GLY | C3074 | 26.990 | 14.201 | 21.354 | 1.00 | 50.33 | C | |
| ATOM | 2346 | N | PHE | C3075 | 25.218 | 13.780 | 19.755 | 1.00 | 49.73 | N | |
| ATOM | 2347 | CA | PHE | C3075 | 23.894 | 13.345 | 19.328 | 1.00 | 49.39 | C | |
| ATOM | 2348 | CB | PHE | C3075 | 23.740 | 13.548 | 17.818 | 1.00 | 49.61 | C | |
| ATOM | 2349 | CG | PHE | C3075 | 23.196 | 14.902 | 17.447 | 1.00 | 50.02 | C | |
| ATOM | 2350 | COl | PHE | C3075 | 23.555 | 16.036 | 18.178 | 1.00 | 50.35 | C | |
| ATOM | 2351 | CG2 | PHE | C3075 | 22.316 | 15.045 | 16.382 | 1.00 | 50.13 | C | |
| ATOM | 2352 | CBl | PHE | C3075 | 23.040 | 17.294 | 17.854 | 1.00 | 50.43 | C | |
| ATOM | 2353 | CB2 | PHE | C3075 | 21.794 | 16.297 | 16.047 | 1.00 | 50.56 | C | |
| ATOM | 2354 | CZ | PHE | C3075 | 22.156 | 17.424 | 16.784 | 1.00 | 50.60 | C | |
| ATOM | 2355 | C | PHE | C3075 | 23.644 | 11.894 | 19.710 | 1.00 | 48.79 | C | |
| ATOM | 2356 | O | PHE | C3075 | 22.530 | 11.528 | 20.073 | 1.00 | 48.53 | O | |
| ATOM | 2357 | N | GLN | C3076 | 24.694 | 11.082 | 19.642 | 1.00 | 48.39 | N | |
| ATOM | 2358 | CA | GLN | C3076 | 24.602 | 9.671 | 19.998 | 1.00 | 47.89 | C | |
| ATOM | 2359 | CB | GLN | C3076 | 25.905 | 8.944 | 19.643 | 1.00 | 48.47 | C | |
| ATOM | 2360 | CG | GLN | C3076 | 26.287 | 9.049 | 18.175 | 1.00 | 49.29 | C | |
| ATOM | 2361 | CD | GLN | C3076 | 25.242 | 8.441 | 17.260 | 1.00 | 49.81 | C | |
| ATOM | 2362 | OE1 | GLN | C3076 | 25.206 | 8.731 | 16.065 | 1.00 | 50.75 | O | |
| ATOM | 2363 | NE2 | GLN | C3076 | 24.391 | 7.584 | 17.815 | 1.00 | 49.76 | N | |
| ATOM | 2364 | C | GLN | C3076 | 24.351 | 9.560 | 21.492 | 1.00 | 47.10 | C | |
| ATOM | 2365 | 0 | GLN | C3076 | 24.003 | 8.493 | 22.000 | 1.00 | 47.45 | O | |
| ATOM | 2366 | N | ASP | C3077 | 24.533 | 10.676 | 22.190 | 1.00 | 45.97 | N | |
| ATOM | 2367 | CA | ASP | C3077 | 24.331 | 10.727 | 23.631 | 1.00 | 44.90 | C | |
| ATOM | 2368 | CB | ASP | C3077 | 25.397 | 11.612 | 24.271 | 1.00 | 46.44 | C | |
| ATOM | 2369 | CG | ASP | C3077 | 26.800 | 11.083 | 24.036 | 1.00 | 47.82 | C | |
| ATOM | 2370 | 001 | ASP | C3077 | 27.081 | 9.951 | 24.499 | 1.00 | 48.20 | O | |
| ATOM | 2371 | 002 | ASP | C3077 | 27.610 | 11.794 | 23.388 | 1.00 | 48.48 | O | |
| ATOM | 2372 | C | ASP | C3077 | 22.950 | 11.252 | 23.975 | 1.00 | 43.23 | C | |
| ATOM | 2373 | 0 | ASP | C3077 | 22.557 | 11.280 | 25.140 | 1.00 | 43.47 | O | |
| ATOM | 2374 | N | LEU | C3078 | 22.219 | 11.675 | 22.950 | 1.00 | 41.15 | N | |
| ATOM | 2375 | CA | LEU | C3078 | 20.869 | 12.193 | 23.129 | 1.00 | 38.63 | C | |
| ATOM | 2376 | CB | LELJ C3078 | 20.754 | 13.610 | 22.558 | 1.00 | 38.12 | C | | |
| ATOM | 2377 | CG | LEU | C3078 | 21.787 | 14.645 | 23.005 | 1.00 | 37.91 | C | |
| ATOM | 2378 | COl | LEU | C3078 | 21.511 | 15.974 | 22.313 | 1.00 | 37.65 | C | |
| ATOM | 2379 | CG2 | LEU | C3078 | 21.735 | 14.801 | 24.508 | 1.00 | 37.75 | C | |
| ATOM | 2380 | C | LEU | C3078 | 19.921 | 11.272 | 22.376 | 1.00 | 36.97 | C | |
| ATOM | 2381 | 0 | LEU | C3078 | 20.225 | 10.833 | 21.269 | 1.00 | 36.76 | O | |
| ATOM | 2382 | N | GLU | C3079 | 18.778 | 10.974 | 22.979 | 1.00 | 34.99 | N | |
| ATOM | 2383 | CA | GLU | C3079 | 17.793 | 10.112 | 22.338 | 1.00 | 33.02 | C | |
| ATOM | 2384 | CB | GLU | C3079 | 17.937 | 8.676 | 22.842 | 1.00 | 33.31 | C | |
| ATOM | 2385 | CO | GLU | C3079 | 16.818 | 7.753 | 22.412 | 1.00 | 34.12 | C | |
| ATOM | 2386 | CD | GLU | C3079 | 17.129 | 6.298 | 22.691 | 1.00 | 34.85 | C | |
| ATOM | 2387 | OE1 | GLU | C3079 | 17.897 | 5.701 | 21.908 | 1.00 | 34.18 | O | |
| ATOM | 2388 | OE2 | GLU | C3079 | 16.616 | 5.754 | 23.699 | 1.00 | 35.70 | O | |
| ATOM | 2389 | C | GLU | C3079 | 16.365 | 10.598 | 22.566 | 1.00 | 31.51 | C | |
| ATOM | 2390 | 0 | GLU | C3079 | 15.955 | 10.880 | 23.695 | 1.00 | 31.00 | O | |
| ATOM | 2391 | N | VAL | C3080 | 15.618 | 10.707 | 21.474 | 1.00 | 29.65 | N | |
| ATOM | 2392 | CA | VAL | C3080 | 14.237 | 11.135 | 21.542 | 1.00 | 28.33 | C | |
| ATOM | 2393 | CB | VAL | C3080 | 13.987 | 12.401 | 20.698 | 1.00 | 28.04 | C | |
| ATOM | 2394 | CG1 | VAL | C3080 | 12.497 | 12.701 | 20.643 | 1.00 | 27.55 | C | |
| ATOM | 2395 | CG2 | VAL | C3080 | 14.739 | 13.576 | 21.289 | 1.00 | 27.56 | C | |
| ATOM | 2396 | C | VAL | C3080 | 13.373 | 10.014 | 21.007 | 1.00 | 28.05 | C | |
| ATOM | 2397 | 0 | VAL | C3080 | 13.518 | 9.600 | 19.858 | 1.00 | 27.43 | O | |
| ATOM | 2398 | N | LEU | C3081 | 12.482 | 9.513 | 21.853 | 1.00 | 27.62 | N | |
| ATOM | 2399 | CA | LEU | C3081 | 11.578 | 8.445 | 21.455 | 1.00 | 27.82 | C | |
| ATOM | 2400 | CB | LEU | C3081 | 11.724 | 7.235 | 22.391 | 1.00 | 27.48 | C | |
| ATOM | 2401 | CG | LEU | C3081 | 13.107 | 6.575 | 22.533 | 1.00 | 27.65 | C | |
| ATOM | 2402 | CD1 | LEU | C3081 | 13.069 | 5.544 | 23.648 | 1.00 | 26.80 | C | |
| ATOM | 2403 | CG2 | LEU | C3081 | 13.534 | 5.928 | 21.223 | 1.00 | 26.81 | C | |
| ATOM | 2404 | C | LEU | C3081 | 10.160 | 9.000 | 21.528 | 1.00 | 27.94 | C | |
| ATOM | 2405 | 0 | LEU | C3081 | 9.965 | 10.194 | 21.744 | 1.00 | 27.66 | O | |
| ATOM | 2406 | N | ASN | C3082 | 9.177 | 8.128 | 21.340 | 1.00 | 28.22 | N | |
| ATOM | 2407 | CA | ASN | C3082 | 7.775 | 8.523 | 21.400 | 1.00 | 28.52 | C | |
| ATOM | 2408 | CB | ASN | C3082 | 7.111 | 8.355 | 20.024 | 1.00 | 28.54 | C | |
| ATOM | 2409 | CO | ASN | C3082 | 7.584 | 9.382 | 19.021 | 1.00 | 28.73 | C | |
| ATOM | 2410 | 001 | ASN | C3082 | 7.295 | 10.569 | 19.148 | 1.00 | 29.62 | O | |
| ATOM | 2411 | ND2 | ASN | C3082 | 8.321 | 8.932 | 18.020 | 1.00 | 29.32 | N | |
| ATOM | 2412 | C | ASN | C3082 | 7.053 | 7.647 | 22.422 | 1.00 | 28.28 | C | |
| ATOM | 2413 | O | ASN | C3082 | 7.199 | 6.421 | 22.407 | 1.00 | 28.06 | O | |
| ATOM | 2414 | N | ASN | C3083 | 6.280 | 8.274 | 23.308 | 1.00 | 27.85 | N | |
| ATOM | 2415 | CA | ASN | C3083 | 5.537 | 7.520 | 24.304 | 1.00 | 27.66 | C | |
| ATOM | 2416 | CB | ASM C3083 | 5.183 | 8.398 | 25.515 | 1.00 | 27.64 | C | | |
| ATOM | 2417 | CG | ASN | C3083 | 4.203 | 9.527 | 25.189 | 1.00 | 27.67 | C | |
| ATOM | 2418 | ODi | ASN | C3083 | 3.949 | 10.384 | 26.033 | 1.00 | 28.51 | O | |
| ATOM | 2419 | ND2 | ASN | C3083 | 3.648 | 9.527 | 23.986 | 1.00 | 27.19 | N | |
| ATOM | 2420 | C | ASN | C3083 | 4.287 | 6.942 | 23.664 | 1.00 | 27.84 | C | |
| ATOM | 2421 | 0 | ASN | C3083 | 3.988 | 7.233 | 22.501 | 1.00 | 27.10 | O | |
| ATOM | 2422 | N | GLU | C3084 | 3.564 | 6.120 | 24.419 | 1.00 | 28.22 | N | |
| ATOM | 2423 | CA | GLU | C3084 | 2.354 | 5.483 | 23.910 | 1.00 | 28.64 | C | |
| ATOM | 2424 | CB | GLU | C3084 | 1.655 | 4.679 | 25.018 | 1.00 | 29.96 | C | |
| ATOM | 2425 | CG | GLU | C3084 | 1.146 | 5.509 | 26.187 | 1.00 | 31.56 | C | |
| ATOM | 2426 | CD | GLU | C3084 | 2.225 | 5.820 | 27.210 | 1.00 | 33.17 | C | |
| ATOM | 2427 | OE1 | GLU | C3084 | 3.404 | 5.994 | 26.821 | 1.00 | 33.53 | O | |
| ATOM | 2428 | OE2 | GLU | C3084 | 1.888 | 5.903 | 28.411 | 1.00 | 34.64 | O | |
| ATOM | 2429 | C | GLU | C3084 | 1.367 | 6.471 | 23.291 | 1.00 | 28.20 | C | |
| ATOM | 2430 | 0 | GLU | C3084 | 0.559 | 6.092 | 22.446 | 1.00 | 28.01 | O | |
| ATOM | 2431 | N | ARG | C3085 | 1.435 | 7.734 | 23.702 | 1.00 | 27.98 | N | |
| ATOM | 2432 | CA | ARG | C3085 | 0.526 | 8.745 | 23.165 | 1.00 | 27.80 | C | |
| ATOM | 2433 | CB | ARG | C3085 | 0.270 | 9.847 | 24.201 | 1.00 | 28.19 | C | |
| ATOM | 2434 | CG | ARG | C3085 | −0.474 | 9.355 | 25.427 | 1.00 | 29.28 | C | |
| ATOM | 2435 | CD | ARG | C3085 | −1.296 | 10.454 | 26.109 | 1.00 | 30.50 | C | |
| ATOM | 2436 | NE | ARG | C3085 | −2.245 | 9.862 | 27.052 | 1.00 | 31.26 | N | |
| ATOM | 2437 | CZ | ARG | C3085 | −1.872 | 9.221 | 28.153 | 1.00 | 32.48 | C | |
| ATOM | 2438 | N | 111 | ARG | C3085 | −0.581 | 9.110 | 28.437 | 1.00 | 33.70 | N |
| ATOM | 2439 | NH2 | ARG | C3085 | −2.770 | 8.663 | 28.954 | 1.00 | 33.05 | N | |
| ATOM | 2440 | C | ARG | C3085 | 1.021 | 9.369 | 21.863 | 1.00 | 27.34 | C | |
| ATOM | 2441 | 0 | ARG | C3085 | 0.310 | 10.158 | 21.244 | 1.00 | 27.35 | O | |
| ATOM | 2442 | N | CL? C3086 | 2.237 | 9.016 | 21.454 | 1.00 | 26.82 | N | | |
| ATOM | 2443 | CA | GLY | C3086 | 2.788 | 9.545 | 20.218 | 1.00 | 25.87 | C | |
| ATOM | 2444 | C | GLY | C3086 | 3.607 | 10.808 | 20.384 | 1.00 | 25.74 | C | |
| ATOM | 2445 | 0 | GLY | C3086 | 4.109 | 11.370 | 19.401 | 1.00 | 25.82 | O | |
| ATOM | 2446 | N | ALA | C3087 | 3.753 | 11.254 | 21.626 | 1.00 | 24.95 | N | |
| ATOM | 2447 | CA | ALA | C3087 | 4.508 | 12.461 | 21.920 | 1.00 | 24.57 | C | |
| ATOM | 2448 | CB | ALA | C3087 | 3.933 | 13.147 | 23.155 | 1.00 | 24.64 | C | |
| ATOM | 2449 | C | ALA | C3087 | 5.991 | 12.183 | 22.130 | 1.00 | 24.46 | C | |
| ATOM | 2450 | 0 | ALA | C3087 | 6.372 | 11.277 | 22.878 | 1.00 | 24.54 | O | |
| ATOM | 2451 | N | PRO | | C3088 | 6.851 | 12.964 | 21.461 | 1.00 | 24.09 | N |
| ATOM | 2452 | CD | PRO | | C3088 | 6.501 | 14.011 | 20.482 | 1.00 | 23.71 | C |
| ATOM | 2453 | CA | PRO | | C3088 | 8.306 | 12.815 | 21.572 | 1.00 | 23.76 | C |
| ATOM | 2454 | CB | PRO | | C3088 | 8.825 | 13.714 | 20.451 | 1.00 | 23.93 | C |
| ATOM | 2455 | CG | PRO | | C3088 | 7.781 | 14.816 | 20.403 | 1.00 | 23.82 | C |
| ATOM | 2456 | C | PRO | | C3088 | 8.799 | 13.249 | 22.952 | 1.00 | 23.43 | C |
| ATOM | 2457 | 0 | PRO | | C3088 | 8.162 | 14.066 | 23.610 | 1.00 | 23.03 | O |
| ATOM | 2458 | N | TYR | C3089 | 9.925 | 12.692 | 23.391 | 1.00 | 23.74 | N | |
| ATOM | 2459 | CA | TYR | C3089 | 10.496 | 13.028 | 24.698 | 1.00 | 24.43 | C | |
| ATOM | 2460 | CB | TYR | C3089 | 9.671 | −12.400 | 25.834 | 1.00 | 24.30 | C | |
| ATOM | 2461 | CG | TYR | C3089 | 9.843 | 10.904 | 25.973 | 1.00 | 24.64 | C | |
| ATOM | 2462 | COl | TYR | C3089 | 10.832 | 10.363 | 26.804 | 1.00 | 24.62 | C | |
| ATOM | 2463 | CE1 | TYR | C3089 | 11.008 | 8.978 | 26.911 | 1.00 | 24.92 | C | |
| ATOM | 2464 | CG2 | TYR | C3089 | 9.033 | 10.027 | 25.256 | 1.00 | 24.84 | C | |
| ATOM | 2465 | CB2 | TYR | C3089 | 9.199 | 8.643 | 25.352 | 1.00 | 25.43 | C | |
| ATOM | 2466 | CZ | TYR | C3089 | 10.187 | 8.125 | 26.178 | 1.00 | 25.32 | C | |
| ATOM | 2467 | OH | TYR | C3089 | 10.353 | 6.761 | 26.236 | 1.00 | 25.52 | O | |
| ATOM | 2468 | C | TYR | C3089 | 11.925 | 12.521 | 24.772 | 1.00 | 24.85 | C | |
| ATOM | 2469 | 0 | TYR | C3089 | 12.279 | 11.552 | 24.103 | 1.00 | 25.10 | O | |
| ATOM | 2470 | N | PHE | C3090 | 12.750 | 13.171 | 25.584 | 1.00 | 25.52 | N | |
| ATOM | 2471 | CA | PHE | C3090 | 14.135 | 12.742 | 25.714 | 1.00 | 26.19 | C | |
| ATOM | 2472 | GB | PHE | C3090 | 15.038 | 13.901 | 26.156 | 1.00 | 26.18 | C | |
| ATOM | 2473 | CG | PHE | C3090 | 15.387 | 14.852 | 25.051 | 1.00 | 26.76 | C | |
| ATOM | 2474 | CD1 | PHE | C3090 | 14.541 | 15.912 | 24.725 | 1.00 | 27.13 | C | |
| ATOM | 2475 | CG2 | PHE | C3090 | 16.554 | 14.675 | 24.311 | 1.00 | 27.22 | C | |
| ATOM | 2476 | CBl | PHE | C3090 | 14.851 | 16.792 | 23.675 | 1.00 | 27.19 | C | |
| ATOM | 2477 | CB2 | PHE | C3090 | 16.882 | 15.548 | 23.253 | 1.00 | 27.68 | C | |
| ATOM | 2478 | CZ | PHE | C3090 | 16.025 | 16.608 | 22.936 | 1.00 | 27.50 | C | |
| ATOM | 2479 | C | PHE | C3090 | 14.277 | 11.595 | 26.693 | 1.00 | 26.31 | C | |
| ATOM | 2480 | 0 | PHE | C3090 | 14.076 | 11.767 | 27.895 | 1.00 | 27.02 | O | |
| ATOM | 2481 | N | SER | C3091 | 14.616 | 10.423 | 26.169 | 1.00 | 26.64 | N | |
| ATOM | 2482 | CA | SER | C3091 | 14.818 | 9.237 | 26.994 | 1.00 | 27.16 | C | |
| ATOM | 2483 | GB | SER | C3091 | 14.549 | 7.964 | 26.185 | 1.00 | 26.45 | C | |
| ATOM | 2484 | OG | SER | C3091 | 15.280 | 7.970 | 24.974 | 1.00 | 27.09 | O | |
| ATOM | 2485 | C | SER | C3091 | 16.256 | 9.235 | 27.513 | 1.00 | 27.57 | C | |
| ATOM | 2486 | 0 | SER | C3091 | 16.594 | 8.485 | 28.424 | 1.00 | 27.76 | O | |
| ATOM | 2487 | N | GLN | C3092 | 17.095 | 10.086 | 26.928 | 1.00 | 28.18 | N | |
| ATOM | 2488 | CA | GLN | C3092 | 18.490 | 10.199 | 27.340 | 1.00 | 29.06 | C | |
| ATOM | 2489 | GB | GLN | C3092 | 19.355 | 9.181 | 26.595 | 1.00 | 30.20 | C | |
| ATOM | 2490 | CG | GLN | C3092 | 20.843 | 9.363 | 26.840 | 1.00 | 31.72 | C | |
| ATOM | 2491 | CD | GLN | C3092 | 21.280 | 8.873 | 28.211 | 1.00 | 32.79 | C | |
| ATOM | 2492 | OE1 | GLN | C3092 | 21.336 | 7.667 | 28.461 | 1.00 | 33.66 | O | |
| ATOM | 2493 | NE2 | GLN | C3092 | 21.590 | 9.805 | 29.105 | 1.00 | 33.12 | N | |
| ATOM | 2494 | C | GLN | C3092 | 19.021 | 11.601 | 27.069 | 1.00 | 28.89 | C | |
| ATOM | 2495 | 0 | GLN | C3092 | 18.889 | 12.118 | 25.954 | 1.00 | 29.04 | O | |
| ATOM | 2496 | N | ALA | C3093 | 19.630 | 12.207 | 28.088 | 1.00 | 28.63 | N | |
| ATOM | 2497 | CA | ALA | C3093 | 20.179 | 13.556 | 27.963 | 1.00 | 28.35 | C | |
| ATOM | 2498 | GB | ALA | C3093 | 19.040 | 14.565 | 27.805 | 1.00 | 28.23 | C | |
| ATOM | 2499 | C | ALA | C3093 | 21.047 | 13.945 | 29.159 | 1.00 | 28.01 | C | |
| ATOM | 2500 | 0 | ALA | C3093 | 20.721 | 13.635 | 30.307 | 1.00 | 28.15 | O | |
| ATOM | 2501 | N | PRO | | C3094 | 22.167 | 14.640 | 28.905 | 1.00 | 27.81 | N |
| ATOM | 2502 | CD | PRO | | C3094 | 22.702 | 15.035 | 27.590 | 1.00 | 27.69 | C |
| ATOM | 2503 | CA | PRO | | C3094 | 23.066 | 15.067 | 29.984 | 1.00 | 27.41 | C |
| ATOM | 2504 | GB | PRO | | C3094 | 24.271 | 15.625 | 29.226 | 1.00 | 27.35 | C |
| ATOM | 2505 | CG | PRO | | C3094 | 23.658 | 16.148 | 27.960 | 1.00 | 27.34 | C |
| ATOM | 2506 | C | PRO | | C3094 | 22.409 | 16.108 | 30.883 | 1.00 | 27.24 | C |
| ATOM | 2507 | 0 | PRO | | C3094 | 22.992 | 17.150 | 31.172 | 1.00 | 27.73 | O |
| ATOM | 2508 | N | PHE | C3095 | 21.195 | 15.820 | 31.330 | 1.00 | 26.89 | N | |
| ATOM | 2509 | CA | PHE | . C3095 | 20.457 | 16.740 | 32.182 | 1.00 | 26.88 | C | |
| ATOM | 2510 | GB | PHE | C3095 | 19.603 | 17.680 | 31.314 | 1.00 | 26.34 | C | |
| ATOM | 2511 | CO | PHE | C3095 | 18.986 | 18.820 | 32.081 | 1.00 | 25.69 | C | |
| ATOM | 2512 | COl | PHE | C3095 | 19.789 | 19.707 | 32.796 | 1.00 | 24.82 | C | |
| ATOM | 2513 | CG2 | PHE | C3095 | 17.606 | 18.973 | 32.133 | 1.00 | 24.80 | C | |
| ATOM | 2514 | GEl | PHE | C3095 | 19.224 | 20.722 | 33.556 | 1.00 | 24.83 | C | |
| ATOM | 2515 | CB2 | PHE | C3095 | 17.031 | 19.985 | 32.890 | 1.00 | 24.99 | C | |
| ATOM | 2516 | CZ | PHE | C3095 | 17.841 | 20.862 | 33.605 | 1.00 | 24.98 | C | |
| ATOM | 2517 | C | PHE | G3095 | 19.569 | 15.927 | 33.115 | 1.00 | 27.25 | C | |
| ATOM | 2518 | 0 | PHE | C3095 | 18.924 | 14.983 | 32.684 | 1.00 | 28.50 | O | |
| ATOM | 2519 | N | SER | C3096 | 19.525 | 16.284 | 34.390 | 1.00 | 27.37 | N | |
| ATOM | 2520 | CA | SER | C3096 | 18.710 | 15.530 | 35.332 | 1.00 | 27.95 | C | |
| ATOM | 2521 | GB | SER | C3096 | 19.542 | 15.174 | 36.565 | 1.00 | 28.67 | C | |
| ATOM | 2522 | 00 | SER | C3096 | 20.425 | 16.230 | 36.883 | 1.00 | 30.49 | O | |
| ATOM | 2523 | C | SER | C3096 | 17.403 | 16.202 | 35.754 | 1.00 | 27.66 | C | |
| ATOM | 2524 | 0 | SER | C3096 | 16.663 | 15.665 | 36.577 | 1.00 | 27.25 | O | |
| ATOM | 2525 | N | GLY | C3097 | 17.117 | 17.372 | 35.192 | 1.00 | 26.91 | N | |
| ATOM | 2526 | CA | GLY | C3097 | 15.874 | 18.045 | 35.521 | 1.00 | 26.05 | C | |
| ATOM | 2527 | C | GLY | C3097 | 14.839 | 17.654 | 34.488 | 1.00 | 25.55 | C | |
| ATOM | 2528 | 0 | GLY | C3097 | 15.047 | 16.696 | 33.745 | 1.00 | 25.35 | O | |
| ATOM | 2529 | N | LYS | C3098 | 13.729 | 18.384 | 34.428 | 1.00 | 25.19 | N | |
| ATOM | 2530 | CA | LYS | C3098 | 12.690 | 18.086 | 33.452 | 1.00 | 24.30 | C | |
| ATOM | 2531 | CB | LYS | C3098 | 11.820 | 18.543 | 33.945 | 1.00 | 24.76 | C | |
| ATOM | 2532 | CG | LYS | C3098 | 10.863 | 17.934 | 35.237 | 1.00 | 25.27 | C | |
| ATOM | 2533 | CD | LYS | C3098 | 9.397 | 18.241 | 35.459 | 1.00 | 26.28 | C | |
| ATOM | 2534 | CB | LYS | C3098 | 8.973 | 17.880 | 36.860 | 1.00 | 26.82 | C | |
| ATOM | 2535 | NZ | LYS | C3098 | 9.334 | 16.468 | 37.155 | 1.00 | 28.61 | N | |
| ATOM | 2536 | G | LYS | C3098 | 12.978 | 18.774 | 32.119 | 1.00 | 23.93 | C | |
| ATOM | 2537 | 0 | LYS | C3098 | 13.330 | 19.954 | 32.077 | 1.00 | 23.38 | O | |
| ATOM | 2538 | N | ILE | C3099 | 12.821 | 18.019 | 31.034 | 1.00 | 23.34 | N | |
| ATOM | 2539 | CA | ILE | C3099 | 13.033 | 18.536 | 29.690 | 1.00 | 22.75 | C | |
| ATOM | 2540 | CB | ILE | C3099 | 13.960 | 17.613 | 28.874 | 1.00 | 22.58 | C | |
| ATOM | 2541 | CG2 | ILE | C3099 | 14.345 | 18.292 | 27.558 | 1.00 | 22.52 | C | |
| ATOM | 2542 | CG1 | ILE | C3099 | 15.219 | 17.291 | 29.688 | 1.00 | 22.55 | C | |
| ATOM | 2543 | COl | ILE | C3099 | 16.199 | 16.357 | 28.985 | 1.00 | 22.28 | C | |
| ATOM | 2544 | C | ILE | C3099 | 11.676 | 18.617 | 28.990 | 1.00 | 22.48 | C | |
| ATOM | 2545 | 0 | ILE | C3099 | 11.054 | 17.591 | 28.699 | 1.00 | 22.47 | O | |
| ATOM | 2546 | N | TRP | C3100 | 11.205 | 19.835 | 28.743 | 1.00 | 21.77 | N | |
| ATOM | 2547 | CA | TRP | C3100 | 9.926 | 20.008 | 28.071 | 1.00 | 20.54 | C | |
| ATOM | 2548 | CB | TRP | C3100 | 9.180 | 21.228 | 28.600 | 1.00 | 19.83 | C | |
| ATOM | 2549 | CG | TRP | C3100 | 8.900 | 21.139 | 30.066 | 1.00 | 19.91 | C | |
| ATOM | 2550 | CG2 | TRP | C3100 | 7.806 | 20.455 | 30.694 | 1.00 | 19.80 | C | |
| ATOM | 2551 | CB2 | TRP | C3100 | 7.968 | 20.605 | 32.091 | 1.00 | 19.84 | C | |
| ATOM | 2552 | CB3 | TRP | C3100 | 6.704 | 19.727 | 30.211 | 1.00 | 19.87 | C | |
| ATOM | 2553 | COl | TRP | C3100 | 9.660 | 21.660 | 31.081 | 1.00 | 19.93 | C | |
| ATOM | 2554 | NEl | TRP | C3100 | 9.106 | 21.343 | 32.296 | 1.00 | 19.91 | N | |
| ATOM | 2555 | CZ2 | TRP | C3100 | 7.069 | 20.058 | 33.015 | 1.00 | 19.27 | C | |
| ATOM | 2556 | CZ3 | TRP | C3100 | 5.808 | 19.181 | 31.128 | 1.00 | 19.78 | C | |
| ATOM | 2557 | CH2 | TRP | C3100 | 6.000 | 19.352 | 32.520 | 1.00 | 20.41 | C | |
| ATOM | 2558 | C | TRP | C3100 | 10.239 | 20.174 | 26.611 | 1.00 | 20.42 | C | |
| ATOM | 2559 | 0 | TRP | C3100 | 10.831 | 21.174 | 26.195 | 1.00 | 21.30 | O | |
| ATOM | 2560 | N | LEU | C3101 | 9.845 | 19.173 | 25.838 | 1.00 | 19.69 | N | |
| ATOM | 2561 | CA | LEU | C3101 | 10.097 | 19.143 | 24.416 | 1.00 | 19.35 | C | |
| ATOM | 2562 | CB | LEU | C3101 | 10.903 | 17.891 | 24.072 | 1.00 | 18.36 | C | |
| ATOM | 2563 | CG | LEU | C3101 | 10.885 | 17.499 | 22.592 | 1.00 | 18.24 | C | |
| ATOM | 2564 | CD1 | LEU | C3101 | 11.677 | 18.536 | 21.786 | 1.00 | 16.55 | C | |
| ATOM | 2565 | CG2 | LEU | C3101 | 11.466 | 16.097 | 22.414 | 1.00 | 17.56 | C | |
| ATOM | 2566 | CLEt) C3101 | 8.831 | 19.145 | 23.587 | 1.00 | 19.41 | C | | | |
| ATOM | 2567 | 0 | LEU | C3101 | 7.846 | 18.493 | 23.929 | 1.00 | 19.45 | O | |
| ATOM | 2568 | N | SER | C3102 | 8.860 | 19.885 | 22.490 | 1.00 | 19.43 | N | |
| ATOM | 2569 | CA | SER | C3102 | 7.722 | 19.899 | 21.596 | 1.00 | 19.85 | C | |
| ATOM | 2570 | CB | SER | C3102 | 6.793 | 21.082 | 21.873 | 1.00 | 19.85 | C | |
| ATOM | 2571 | OG | SER | C3102 | 5.550 | 20.887 | 21.212 | 1.00 | 19.01 | O | |
| ATOM | 2572 | C | SER | C3102 | 8.269 | 19.972 | 20.190 | 1.00 | 20.13 | C | |
| ATOM | 2573 | 0 | SER | C3102 | 9.272 | 20.637 | 19.939 | 1.00 | 19.67 | O | |
| ATOM | 2574 | N | ILE | C3103 | 7.624 | 19.251 | 19.282 | 1.00 | 21.04 | N | |
| ATOM | 2575 | CA | ILE | C3103 | 8.037 | 19.231 | 17.889 | 1.00 | 22.10 | C | |
| ATOM | 2576 | CB | ILE | C3103 | 8.735 | 17.906 | 17.523 | 1.00 | 21.92 | C | |
| ATOM | 2577 | CG2 | ILE | C3103 | 9.127 | 17.922 | 16.059 | 1.00 | 21.89 | C | |
| ATOM | 2578 | CG1 | ILE | C3103 | 9.963 | 17.681 | 18.405 | 1.00 | 22.24 | C | |
| ATOM | 2579 | CD1 | ILE | C3103 | 10.695 | 16.376 | 18.094 | 1.00 | 22.36 | C | |
| ATOM | 2580 | C | ILE | C3103 | 6.804 | 19.346 | 17.010 | 1.00 | 23.17 | C | |
| ATOM | 2581 | 0 | ILE | C3103 | 5.753 | 18.805 | 17.338 | 1.00 | 23.73 | O | |
| ATOM | 2582 | N | SER | C3104 | 6.932 | 20.051 | 15.896 | 1.00 | 23.79 | N | |
| ATOM | 2583 | CA | SER | C3104 | 5.829 | 20.186 | 14.965 | 1.00 | 24.82 | C | |
| ATOM | 2584 | CB | SER | C3104 | 4.989 | 21.419 | 15.294 | 1.00 | 24.69 | C | |
| ATOM | 2585 | OG | SER | C3104 | 3.905 | 21.546 | 14.390 | 1.00 | 24.65 | O | |
| ATOM | 2586 | C | SER | c3104 | 6.428 | 20.295 | 13.567 | 1.00 | 25.82 | C | |
| ATOM | 2587 | 0 | SER | C3104 | 7.577 | 20.690 | 13.403 | 1.00 | 25.83 | O | |
| ATOM | 2588 | N | HIS | C3105 | 5.646 | 19.929 | 12.560 | 1.00 | 27.14 | N. | |
| ATOM | 2589 | CA | HIS | C3105 | 6.140 | 19.996 | 11.200 | 1.00 | 28.67 | C | |
| ATOM | 2590 | CB | HIS | C3105 | 6.841 | 18.686 | 10.819 | 1.00 | 30.20 | C | |
| ATOM | 2591 | CG | HIS | C3105 | 5.901 | 17.542 | 10.584 | 1.00 | 31.48 | C | |
| ATOM | 2592 | CG2 | HIS | C3105 | 5.474 | 16.961 | 9.437 | 1.00 | 32.52 | C | |
| ATOM | 2593 | ND1 | HIS | C3105 | 5.252 | 16.887 | 11.607 | 1.00 | 33.00 | N | |
| ATOM | 2594 | CBl | HIS | C3105 | 4.465 | 15.952 | 11.103 | 1.00 | 33.08 | C | |
| ATOM | 2595 | NE2 | HIS | C3105 | 4.580 | 15.977 | 9.787 | 1.00 | 33.14 | N | |
| ATOM | 2596 | C | HIS | C3105 | 5.046 | 20.253 | 10.187 | 1.00 | 29.03 | C | |
| ATOM | 2597 | 0 | HIS | C3105 | 3.862 | 20.082 | 10.468 | 1.00 | 28.87 | O | |
| ATOM | 2598 | N | THR | C3106 | 5.478 | 20.682 | 9.009 | 1.00 | 29.70 | N | |
| ATOM | 2599 | CA | THR | C3106 | 4.609 | 20.918 | 7.871 | 1.00 | 30.87 | C | |
| ATOM | 2600 | CB | THR | C3106 | 4.542 | 22.416 | 7.492 | 1.00 | 30.95 | C | |
| ATOM | 2601 | OGi | THR | C3106 | 5.853 | 22.895 | 7.185 | 1.00 | 31.53 | O | |
| ATOM | 2602 | CG2 | THR | C3106 | 3.970 | 23.231 | 8.631 | 1.00 | 31.66 | C | |
| ATOM | 2603 | C | THR | C3106 | 5.310 | 20.118 | 6.758 | 1.00 | 31.46 | C | |
| ATOM | 2604 | 0 | THR | C3106 | 6.288 | 19.411 | 7.032 | 1.00 | 31.17 | O | |
| ATOM | 2605 | N | ASP | C3107 | 4.834 | 20.223 | 5.521 | 1.00 | 32.04 | N | |
| ATOM | 2606 | CA | ASP | C3107 | 5.444 | 19.475 | 4.423 | 1.00 | 32.74 | C | |
| ATOM | 2607 | CD | ASP | C3107 | 4.584 | 19.570 | 3.151 | 1.00 | 33.89 | C | |
| ATOM | 2608 | CG | ASP | C3107 | 3.252 | 18.836 | 3.276 | 1.00 | 35.29 | C | |
| ATOM | 2609 | ODi | ASP | C3107 | 3.059 | 18.077 | 4.257 | 1.00 | 35.09 | O | |
| ATOM | 2610 | 002 | ASP | C3107 | 2.396 | 19.017 | 2.379 | 1.00 | 35.84 | O | |
| ATOM | 2611 | C | ASP | C3107 | 6.861 | 19.942 | 4.096 | 1.00 | 32.51 | C | |
| ATOM | 2612 | 0 | ASP | C3107 | 7.680 | 19.158 | 3.609 | 1.00 | 32.63 | O | |
| ATOM | 2613 | NGL~I C3108 | 7.152 | 21.210 | 4.371 | 1.00 | 31.99 | N | | | |
| ATOM | 2614 | CA | GLN | C3108 | 8.467 | 21.768 | 4.068 | 1.00 | 31.59 | C | |
| ATOM | 2615 | CR | GLN | C3108 | 8.310 | 23.119 | 3.362 | 1.00 | 32.00 | C | |
| ATOM | 2616 | CG | GLN | C3108 | 7.482 | 23.077 | 2.086 | 1.00 | 32.40 | | |
| ATOM | 2617 | CD | GLN | C3108 | 7.294 | 24.452 | 1.469 | 0.05 | 32.28 | C | |
| ATOM | 2618 | OE1 | GLN | C3108 | 6.650 | 24.597 | 0.430 | 0.05 | 32.30 | O | |
| ATOM | 2619 | NE2 | GLN | C3108 | 7.856 | 25.469 | 2.110 | 0.05 | 32.28 | N | |
| ATOM | 2620 | C | GLN | C3108 | 9.391 | 21.957 | 5.272 | 1.00 | 30.94 | C | |
| ATOM | 2621 | 0 | GLN | C3108 | 10.610 | 21.833 | 5.145 | 1.00 | 30.85 | O | |
| ATOM | 2622 | N | PHE | . C3109 | 8.822 | 22.258 | 6.436 | 1.00 | 30.12 | N | |
| ATOM | 2623 | CA | PHE | C3109 | 9.648 | 22.489 | 7.610 | 1.00 | 29.07 | C | |
| ATOM | 2624 | CB | PHE | C3109 | 9.652 | 23.980 | 7.953 | 1.00 | 29.62 | C | |
| ATOM | 2625 | CG | PHE | C3109 | 10.111 | 24.857 | 6.827 | 1.00 | 30.64 | C | |
| ATOM | 2626 | COl | PHE | C3109 | 9.215 | 25.275 | 5.845 | 1.00 | 31.09 | C | |
| ATOM | 2627 | CG2 | PHE | C3109 | 11.440 | 25.259 | 6.738 | 1.00 | 30.59 | C | |
| ATOM | 2628 | CBl | PHE | C3109 | 9.636 | 26.084 | 4.788 | 1.00 | 31.21 | C | |
| ATOM | 2629 | CB2 | PHE | C3109 | 11.870 | 26.066 | 5.688 | 1.00 | 30.89 | C | |
| ATOM | 2630 | CZ | PHE | C3109 | 10.964 | 26.480 | 4.709 | 1.00 | 31.08 | C | |
| ATOM | 2631 | C | PHE | C3109 | 9.292 | 21.716 | 8.860 | 1.00 | 28.01 | C | |
| ATOM | 2632 | 0 | PHE | C3109 | 8.252 | 21.065 | 8.936 | 1.00 | 28.14 | O | |
| ATOM | 2633 | N | VAL | C3110 | 10.192 | 21.798 | 9.838 | 1.00 | 26.84 | N | |
| ATOM | 2634 | CA | VAL | C3110 | 10.019 | 21.175 | 11.140 | 1.00 | 25.60 | C | |
| ATOM | 2635 | CB | VAL | C3110 | 10.766 | 19.822 | 11.250 | 1.00 | 25.63 | C | |
| ATOM | 2636 | CG1 | VAL | C3110 | 12.268 | 20.035 | 11.191 | 1.00 | 25.54 | C | |
| ATOM | 2637 | CG2 | VAL | C3110 | 10.387 | 19.133 | 12.554 | 1.00 | 25.58 | C | |
| ATOM | 2638 | C | VAL | C3110 | 10.580 | 22.139 | 12.187 | 1.00 | 24.84 | C | |
| ATOM | 2639 | 0 | VAL | C3110 | 11.596 | 22.798 | 11.957 | 1.00 | 24.75 | O | |
| ATOM | 2640 | N | THR | C3111 | 9.911 | 22.239 | 13.328 | 1.00 | 23.62 | N | |
| ATOM | 2641 | CA | THR | C3111 | 10.377 | 23.120 | 14.395 | 1.00 | 23.00 | C | |
| ATOM | 2642 | CB | THR | C3111 | 9.451 | 24.332 | 14.583 | 1.00 | 23.10 | C | |
| ATOM | 2643 | OGi | THR | C3111 | 8.138 | 23.871 | 14.924 | 1.00 | 24.21 | O | |
| ATOM | 2644 | CG2 | THR | C3111 | 9.369 | 25.145 | 13.306 | 1.00 | 23.43 | C | |
| ATOM | 2645 | C | THR | C3111 | 10.424 | 22.363 | 15.712 | 1.00 | 22.01 | C | |
| ATOM | 2646 | 0 | THR | C3111 | 9.616 | 21.466 | 15.951 | 1.00 | 22.10 | O | |
| ATOM | 2647 | N | ALA | C3122 | 11.382 | 22.726 | 16.554 | 1.00 | 21.05 | N | |
| ATOM | 2648 | CA | ALA | C3112 | 11.532 | 22.110 | 17.859 | 1.00 | 20.79 | C | |
| ATOM | 2649 | CB | ALA | C3112 | 12.710 | 21.135 | 17.846 | 1.00 | 20.00 | C | |
| ATOM | 2650 | C | ALA | C3112 | 11.741 | 23.194 | 18.927 | 1.00 | 20.81 | C | |
| ATOM | 2651 | 0 | ALA | C3112 | 12.367 | 24.216 | 18.672 | 1.00 | 20.76 | O | |
| ATOM | 2652 | N | SER | C3113 | 11.199 | 22.968 | 20.118 | 1.00 | 20.90 | N | |
| ATOM | 2653 | CA | SER | C3113 | 11.346 | 23.909 | 21.215 | 1.00 | 20.61 | C | |
| ATOM | 2654 | CB | SER | C3113 | 10.061 | 24.721 | 21.413 | 1.00 | 20.72 | C | |
| ATOM | 2655 | OG | SER | C3113 | 10.176 | 25.598 | 22.526 | 1.00 | 20.34 | O | |
| ATOM | 2656 | C | SER | C3113 | 11.653 | 23.128 | 22.477 | 1.00 | 20.63 | C | |
| ATOM | 2657 | 0 | SER | C3113 | 11.013 | 22.119 | 22.759 | 1.00 | 20.86 | O | |
| ATOM | 2658 | N | VAL | C3114 | 12.633 | 23.599 | 23.237 | 1.00 | 20.54 | N | |
| ATOM | 2659 | CA | VAL | C3114 | 13.019 | 22.938 | 24.463 | 1.00 | 20.40 | C | |
| ATOM | 2660 | CB | VAL | C3114 | 14.349 | 22.188 | 24.288 | 1.00 | 20.41 | C | |
| ATOM | 2661 | COl | VAL | C3114 | 14.872 | 21.743 | 25.647 | 1.00 | 19.44 | C | |
| ATOM | 2662 | CG2 | VAL | C3114 | 14.148 | 20.976 | 23.357 | 1.00 | 20.11 | C | |
| ATOM | 2663 | C | VAL | C3114 | 13.159 | 23.883 | 25.644 | 1.00 | 20.99 | C | |
| ATOM | 2664 | 0 | VAL | C3114 | 13.804 | 24.938 | 25.552 | 1.00 | 20.99 | O | |
| ATOM | 2665 | N | ILE | C3115 | 12.543 | 23.494 | 26.756 | 1.00 | 21.35 | N | |
| ATOM | 2666 | CA | ILE | C3115 | 12.626 | 24.269 | 27.980 | 1.00 | 22.44 | C | |
| ATOM | 2667 | CB | ILE | C3115 | 11.250 | 24.816 | 28.428 | 1.00 | 22.27 | C | |
| ATOM | 2668 | CG2 | ILE | C3115 | 11.440 | 25.821 | 29.562 | 1.00 | 22.27 | C | |
| ATOM | 2669 | CG1 | ILE | C3115 | 10.553 | 25.529 | 27.262 | 1.00 | 22.90 | C | |
| ATOM | 2670 | COl | ILE | C3115 | 9.141 | 25.990 | 27.562 | 1.00 | 22.49 | C | |
| ATOM | 2671 | C | ILE | C3115 | 13.167 | 23.360 | 29.077 | 1.00 | 23.09 | C | |
| ATOM | 2672 | 0 | ILE | C3115 | 12.570 | 22.331 | 29.388 | 1.00 | 23.11 | O | |
| ATOM | 2673 | N | LEU | C3116 | 14.311 | 23.730 | 29.640 | 1.00 | 23.90 | N | |
| ATOM | 2674 | CA | LEU | C3116 | 14.912 | 22.959 | 30.720 | 1.00 | 25.63 | C | |
| ATOM | 2675 | CB | LEU | C3116 | 16.434 | 23.051 | 30.667 | 1.00 | 24.96 | C | |
| ATOM | 2676 | CG | LEU | C3116 | 17.007 | 22.497 | 29.366 | 1.00 | 24.50 | C | |
| ATOM | 2677 | COl | LEU | C3116 | 18.508 | 22.663 | 29.350 | 1.00 | 23.62 | C | |
| ATOM | 2678 | CG2 | LEU | C3116 | 16.596 | 21.029 | 29.236 | 1.00 | 24.58 | C | |
| ATOM | 2679 | C | LEU | C3116 | 14.418 | 23.524 | 32.036 | 1.00 | 27.20 | C | |
| ATOM | 2680 | 0 | LEU | C3116 | 14.412 | 24.743 | 32.236 | 1.00 | 27.34 | O | |
| ATOM | 2681 | N | GLU | C3117 | 13.998 | 22.636 | 32.930 | 1.00 | 28.70 | N | |
| ATOM | 2682 | CA | GLU | C3117 | 13.490 | 23.061 | 34.226 | 1.00 | 30.61 | C | |
| ATOM | 2683 | CB | GLU | C3117 | 11.957 | 22.951 | 34.249 | 1.00 | 30.11 | C | |
| ATOM | 2684 | CG | GLU | C3117 | 11.322 | 23.257 | 35.592 | 1.00 | 30.29 | C | |
| ATOM | 2685 | CD | GLU | C3117 | 9.808 | 23.100 | 35.585 | 1.00 | 30.63 | C | |
| ATOM | 2686 | QEl | GLU | C3117 | 9.313 | 22.085 | 35.043 | 1.00 | 30.65 | O | |
| ATOM | 2687 | OE2 | GLU | C3117 | 9.114 | 23.985 | 36.133 | 1.00 | 30.59 | O | |
| ATOM | 2688 | C | GLU | C3117 | 14.089 | 22.241 | 35.357 | 1.00 | 31.98 | C | |
| ATOM | 2689 | 0 | GLU | C3117 | 14.353 | 21.052 | 35.205 | 1.00 | 32.46 | O | |
| ATOM | 2690 | N | GLU | C3118 | 14.321 | 22.900 | 36.485 | 1.00 | 33.79 | N | |
| ATOM | 2691 | CA | GLU | C3118 | 24.863 | 22.257 | 37.674 | 1.00 | 35.41 | C | |
| ATOM | 2692 | CB | GLU | C3118 | 16.264 | 22.779 | 37.990 | 1.00 | 36.58 | C | |
| ATOM | 2693 | CG | GLU | C3118 | 17.396 | 21.983 | 37.366 | 1.00 | 38.74 | C | |
| ATOM | 2694 | CD | GLU | C3118 | 17.335 | 20.507 | 37.733 | 1.00 | 40.59 | C | |
| ATOM | 2695 | OE1 | GLU | C3118 | 16.936 | 20.189 | 38.881 | 1.00 | 41.34 | O | |
| ATOM | 2696 | OE2 | GLU | C3118 | 17.700 | 19.669 | 36.877 | 1.00 | 41.36 | O | |
| ATOM | 2697 | C | GLU | C3118 | 13.940 | 22.578 | 38.837 | 1.00 | 36.11 | C | |
| ATOM | 2698 | O | GLU | C3118 | 13.351 | 21.632 | 39.403 | 1.00 | 37.15 | O | |
| ATOM | 2699 | OXT | GLU | C3118 | 13.813 | 23.778 | 39.156 | 1.00 | 36.00 | O | |
| TER | 2700 | | GLU | C3118 | | | | | | | |
| ATOM | 2701 | O | HOH | W 1 | 8.576 | 28.003 | 19.046 | 1.00 | 26.06 | O | |
| ATOM | 2702 | O | HOH | W 2 | 11.001 | 13.668 | 14.737 | 1.00 | 16.99 | O | |
| ATOM | 2703 | O | HOH | W 3 | −5.347 | 33.382 | 14.724 | 1.00 | 27.55 | O | |
| ATOM | 2704 | O | HOH | W 4 | 19.615 | 31.388 | 19.825 | 1.00 | 18.58 | O | |
| ATOM | 2705 | O | HOH | W 5 | −11.216 | 36.113 | 18.617 | 1.00 | 38.18 | O | |
| ATOM | 2706 | O | HOH | W 6 | 0.159 | 37.746 | 18.293 | 1.00 | 26.61 | O | |
| ATOM | 2707 | O | HOH | W 7 | 1.276 | 36.190 | 20.543 | 1.00 | 28.54 | O | |
| ATOM | 2708 | O | HOH | W 8 | 12.334 | 34.067 | 2.484 | 1.00 | 24.27 | O | |
| ATOM | 2709 | O | HOH | W 9 | 0.255 | 6.277 | 33.704 | 1.00 | 25.24 | O | |
| ATOM | 2710 | O | HOH | W 1O | 18.182 | 37.167 | 27.027 | 1.00 | 23.05 | O | |
| ATOM | 2711 | O | HOH | W 11 | −4.577 | 31.410 | 31.511 | 1.00 | 38.36 | O | |
| ATOM | 2712 | O | HOH | W 12 | −1.511 | 45.557 | 32.920 | 1.00 | 53.67 | O | |
| ATOM | 2713 | O | HOH | W 13 | 18.448 | 25.653 | 12.095 | 1.00 | 26.48 | O | |
| ATOM | 2714 | O | HOH | W 14 | 3.912 | 2.648 | 29.570 | 1.00 | 20.27 | O | |
| ATOM | 2715 | O | HOH | W 15 | −11.595 | 44.974 | 29.760 | 1.00 | 29.90 | O | |
| ATOM | 2716 | O | HOH | W 16 | 25.183 | 18.367 | 31.463 | 1.00 | 31.92 | O | |
| ATOM | 2717 | O | HOH | W 17 | 1.272 | 28.443 | 2.735 | 1.00 | 24.74 | O | |
| ATOM | 2718 | O | HOH | W 18 | 19.674 | 51.864 | 7.542 | 1.00 | 32.28 | O | |
| ATOM | 2719 | O | HOH | W 19 | 7.406 | 44.679 | 0.671 | 1.00 | 37.76 | O | |
| ATOM | 2720 | O | HOH | W 20 | 9.102 | 25.639 | 41.587 | 1.00 | 37.75 | O | |
| ATOM | 2721 | O | HOH | W 21 | −2.117 | 42.358 | 29.367 | 1.00 | 23.51 | O | |
| ATOM | 2722 | O | HOH | W 22 | 12.144 | 15.091 | 31.453 | 1.00 | 40.21 | O | |
| ATOM | 2723 | O | HOH | W 23 | 20.216 | 37.895 | 14.318 | 1.00 | 23.27 | O | |
| ATOM | 2724 | O | HOH | W 24 | 15.409 | 30.644 | 36.448 | 1.00 | 34.67 | O | |
| ATOM | 2725 | O | HOH | W 25 | 8.888 | 28.186 | 21.855 | 1.00 | 19.15 | O | |
| ATOM | 2726 | O | HOH | W 26 | −6.604 | 42.281 | 29.309 | 1.00 | 52.83 | O | |
| ATOM | 2727 | O | HOH | W 27 | 20.104 | 53.238 | 15.360 | 1.00 | 42.16 | O | |
| ATOM | 2728 | O | HOH | W 28 | 24.519 | 36.311 | 10.567 | 1.00 | 45.76 | O | |
| ATOM | 2729 | O | HOH | W 29 | 17.712 | 23.831 | 7.356 | 1.00 | 49.27 | O | |
| ATOM | 2730 | O | HOH | W 30 | −6.885 | 28.651 | 32.939 | 1.00 | 45.10 | O | |
| ATOM | 2731 | O | HOH | W 31 | −4.869 | 16.926 | 12.390 | 1.00 | 39.22 | O | |
| ATOM | 2732 | O | HOH | W 32 | 20.935 | 28.085 | 34.073 | 1.00 | 42.57 | O | |
| ATOM | 2733 | O | HOH | W 33 | 21.868 | 15.456 | 38.834 | 1.00 | 28.55 | O | |
| ATOM | 2734 | O | HOH | W 34 | 30.171 | 27.602 | 20.593 | 1.00 | 34.40 | O | |
| ATOM | 2735 | O | HOH | W 35 | 20.651 | 33.038 | 6.426 | 1.00 | 38.43 | O | |
| ATOM | 2736 | O | HOH | W 36 | 10.238 | 19.208 | 1.078 | 1.00 | 45.56 | O | |
| ATOM | 2737 | O | HOH | W 37 | 13.283 | 54.499 | 28.613 | 1.00 | 29.72 | O | |
| ATOM | 2738 | O | HOH | W 38 | −11.211 | 32.999 | 2.256 | 1.00 | 31.79 | O | |
| ATOM | 2739 | O | HOH | W 39 | 9.887 | 29.798 | 36.670 | 1.00 | 39.85 | O | |
| ATOM | 2740 | O | HOH | W 40 | 18.546 | 10.765 | 31.009 | 1.00 | 38.20 | O | |
| ATOM | 2741 | O | HOH | W 41 | 24.510 | 24.190 | 30.902 | 1.00 | 36.49 | O | |
| ATOM | 2742 | O | HOH | W 42 | 30.384 | 38.300 | 20.406 | 1.00 | 44.88 | O | |
| ATOM | 2743 | O | HOH | W 43 | 18.885 | 28.685 | 22.975 | 1.00 | 28.61 | O | |
| ATOM | 2744 | O | HOH | W 44 | 2.917 | 33.308 | 36.961 | 1.00 | 38.63 | O | |
| ATOM | 2745 | O | HOH | W 45 | 20.025 | .37.807 | 29.500 | 1.00 | 35.61 | O | |
| ATOM | 2746 | O | HOH | W 46 | 8.253 | 16.151 | 27.102 | 1.00 | 45.55 | O | |
| ATOM | 2747 | O | HOH | W 47 | 7.572 | 21.032 | 36.495 | 1.00 | 16.73 | O | |
| ATOM | 2748 | O | HOH | W 48 | 19.451 | 28.611 | 27.413 | 1.00 | 35.89 | O | |
| ATOM | 2749 | O | HOH | W 49 | 11.476 | 8.815 | 36.574 | 1.00 | 25.09 | O | |
| ATOM | 2750 | O | HOH | W 50 | 14.236 | 27.513 | 3.754 | 1.00 | 34.47 | O | |
| ATOM | 2751 | O | HOH | W 51 | 5.103 | 9.140 | 28.941 | 1.00 | 41.40 | O | |
| ATOM | 2752 | O | HOH | W 52 | 34.054 | 32.834 | 25.976 | 1.00 | 45.36 | O | |
| ATOM | 2753 | O | HOH | W 53 | 12.068 | 21.222 | 2.790 | 1.00 | 39.74 | O | |
| ATOM | 2754 | O | HOH | W 54 | 2.919 | 30.077 | 39.125 | 1.00 | 41.17 | O | |
| ATOM | 2755 | O | HOH | W 55 | 27.605 | 12.175 | 17.892 | 1.00 | 50.62 | O | |
| ATOM | 2756 | O | HOH | W 56 | −12.868 | 26.916 | 30.495 | 1.00 | 43.36 | O | |
| ATOM | 2757 | O | HOH | W 57 | 9.027 | 13.611 | 36.816 | 1.00 | 43.33 | O | |
| ATOM | 2758 | O | HOH | W 58 | 7.507 | 37.992 | 7.686 | 1.00 | 29.16 | O | |
| ATOM | 2759 | O | HOH | W 59 | 18.024 | 26.076 | 4.943 | 1.00 | 45.92 | O | |
| ATOM | 2760 | O | HOH | W 60 | 21.889 | 53.044 | 19.501 | 1.00 | 41.90 | O | |
| ATOM | 2761 | O | HOH | W 61 | 34.566 | 32.372 | 30.692 | 1.00 | 50.00 | O | |
| ATOM | 2762 | O | HOH | W 62 | 5.289 | 23.268 | 4.539 | 1.00 | 26.10 | O | |
| ATOM | 2763 | O | HOH | W 63 | 21.724 | 28.905 | 29.036 | 1.00 | 54.44 | O | |
| ATOM | 2764 | O | HOH | W 64 | 7.000 | 28.471 | 37.399 | 1.00 | 36.37 | O | |
| ATOM | 2765 | O | HOH | W 65 | 31.382 | 30.725 | 21.632 | 1.00 | 32.29 | O | |
| ATOM | 2766 | O | HOH | W 66 | 28.182 | 46.827 | 4.332 | 1.00 | 48.39 | O | |
| ATOM | 2767 | O | HOH | W 67 | 7.410 | 9.794 | 14.247 | 1.00 | 49.84 | O | |
| ATOM | 2768 | O | HOH | W 68 | 31.517 | 25.424 | 22.117 | 1.00 | 43.26 | O | |
| ATOM | 2769 | O | HOH | W 69 | −5.301 | 33.373 | 34.239 | 1.00 | 34.98 | O | |
| ATOM | 2770 | O | HOH | W 70 | 36.871 | 33.074 | 30.281 | 1.00 | 41.21 | O | |
| ATOM | 2771 | O | HOH | W 71 | 24.903 | 41.702 | 17.966 | 1.00 | 33.98 | O | |
| ATOM | 2772 | O | HOH | W 72 | 17.963 | 38.386 | 31.211 | 1.00 | 35.46 | O | |
| ATOM | 2773 | O | HOH | W 73 | 20.723 | 48.845 | 4.797 | 1.00 | 50.12 | O | |
| ATOM | 2774 | O | HOH | W 74 | −8.379 | 30.159 | 31.227 | 1.00 | 58.73 | O | |
| ATOM | 2775 | O | HOH | W 75 | −1.343 | 19.609 | 10.013 | 1.00 | 53.09 | O | |
| ATOM | 2776 | O | HOH | W 76 | 15.642 | 12.801 | 31.165 | 1.00 | 65.31 | O | |
| ATOM | 2777 | O | HOH | W 77 | −14.605 | 25.461 | 6.171 | 1.00 | 36.87 | O | |
| ATOM | 2778 | O | HOH | W 78 | 8.718 | 39.594 | 0.632 | 1.00 | 44.46 | O | |
| ATOM | 2779 | O | HOH | W 79 | 22.426 | 26.766 | 11.895 | 1.00 | 39.57 | O | |
| ATOM | 2780 | O | HOH | W 80 | 1.403 | 12.128 | 29.940 | 1.00 | 41.04 | O | |
| ATOM | 2781 | O | HOH | W 81 | 9.724 | 26.576 | 37.441 | 1.00 | 52.28 | O | |
| ATOM | 2782 | O | HOH | W 82 | −20.852 | 35.093 | 18.162 | 1.00 | 32.23 | O | |
| ATOM | 2783 | O | HOH | W 83 | −22.727 | 33.513 | 17.160 | 1.00 | 46.00 | O | |
| ATOM | 2784 | O | HOH | W 84 | −9.764 | 38.910 | 4.301 | 1.00 | 38.92 | O | |
| ATOM | 2785 | O | HOH | W 85 | 4.067 | 40.345 | 32.364 | 1.00 | 54.10 | O | |
| ATOM | 2786 | O | HOH | W 86 | 8.783 | 44.044 | 3.423 | 1.00 | 45.06 | O | |
| ATOM | 2787 | O | HOH | W 87 | 19.259 | 34.587 | 26.113 | 1.00 | 34.60 | O | |
| ATOM | 2788 | O | HOH | W 88 | 19.168 | 34.051 | 31.232 | 1.00 | 40.61 | O | |
| ATOM | 2789 | O | HOH | W 89 | −17.637 | 26.480 | 8.650 | 1.00 | 35.76 | O | |
| ATOM | 2790 | O | HOH | W 90 | 5.883 | 33.114 | 4.115 | 1.00 | 36.72 | O | |
| ATOM | 2791 | O | HOH | W 91 | 5.401 | 9.271 | 3.439 | 1.00 | 23.12 | O | |
| ATOM | 2792 | O | HOH | W 92 | 16.578 | 55.625 | 9.782 | 1.00 | 34.03 | O | |
| ATOM | 2793 | O | HOH | W 93 | −6.649 | 22.603 | 36.177 | 1.00 | 49.27 | O | |
| ATOM | 2794 | O | HOH | W 94 | 12.263 | 32.477 | 39.013 | 1.00 | 44.31 | O | |
| ATOM | 2795 | O | HOH | W 95 | 11.064 | 51.183 | 10.359 | 1.00 | 46.81 | O | |
| ATOM | 2796 | O | HOH | W 96 | −16.103 | 28.570 | 5.999 | 1.00 | 45.48 | O | |
| ATOM | 2797 | O | HOH | W 97 | 19.096 | 56.580 | 8.669 | 1.00 | 48.52 | O | |
| ATOM | 2798 | O | HOH | W 98 | −0.441 | 38.083 | 25.842 | 1.00 | 30.49 | O | |
| ATOM | 2799 | O | HOH | W 99 | 18.821 | 50.159 | 23.847 | 1.00 | 35.93 | O | |
| ATOM | 2800 | O | HOH | W 100 | 21.593 | 29.439 | 22.517 | 1.00 | 46.08 | O | |
| ATOM | 2801 | O | HOH | W 101 | 24.154 | 31.033 | 26.613 | 1.00 | 41.03 | O | |
| ATOM | 2802 | O | HOH | W 102 | 18.634 | 43.303 | 30.404 | 1.00 | 40.85 | O | |
| ATOM | 2803 | O | HOH | W 103 | 19.981 | 32.247 | 35.107 | 1.00 | 36.43 | O | |
| ATOM | 2804 | O | HOH | W 104 | −10.088 | 29.062 | 1.003 | 1.00 | 36.43 | O | |
| ATOM | 2805 | O | HOH | W 105 | −18.529 | 19.631 | 5.334 | 1.00 | 38.77 | O | |
| ATOM | 2806 | O | HOH | W 106 | −20.984 | 28.385 | 15.095 | 1.00 | 40.22 | O | |
| ATOM | 2807 | O | HOH | W 107 | −19.013 | 26.762 | 15.815 | 1.00 | 32.06 | O | |
| ATOM | 2808 | O | HOH | W 108 | −14.266 | 19.001 | 19.100 | 1.00 | 41.92 | O | |
| ATOM | 2809 | O | HOH | W 109 | 7.301 | 10.6941 | 1.123 | 1.00 | 37.73 | O | |
| ATOM | 2810 | O | HOH | W 110 | 24.398 | 18.549 | 24.386 | 1.00 | 37.13 | O | |
| ATOM | 2811 | O | HOH | W 111 | 2.259 | 41.877 | 17.603 | 1.00 | 33.35 | O | |
| ATOM | 2812 | O | HOH | W 112 | 22.143 | 19.952 | 8.110 | 1.00 | 46.84 | O | |
| ATOM | 2813 | O | HOH | W 113 | −4.490 | 20.867 | 8.897 | 1.00 | 50.63 | O | |
| ATOM | 2814 | O | HOH | W 114 | 0.274 | 4.462 | 29.620 | 1.00 | 46.55 | O | |
| ATOM | 2815 | O | HOH | W 115 | 9.966 | 5.205 | 19.879 | 1.00 | 29.38 | O | |
| ATOM | 2816 | O | HOH | W 116 | 11.609 | 14.420 | 39.214 | 1.00 | 45.17 | O | |
| ATOM | 2817 | O | HOH | W 117 | 21.720 | 11.206 | 36.139 | 1.00 | 23.23 | O | |
| ATOM | 2818 | O | HOH | W 118 | 21.825 | 46.932 | 3.417 | 1.00 | 42.90 | O | |
| ATOM | 2819 | O | HOH | W 119 | 0.067 | 30.700 | 1.199 | 1.00 | 54.52 | O | |
| ATOM | 2820 | O | HOH | W 120 | 10.218 | 22.583 | 38.806 | 1.00 | 43.46 | O | |
| ATOM | 2821 | O | HOH | W 121 | 0.686 | 27.966 | 42.608 | 1.00 | 42.57 | O | |
| ATOM | 2822 | O | HOH | W 122 | 23.786 | 35.976 | 16.687 | 1.00 | 27.98 | O | |
| ATOM | 2823 | O | HOH | W 123 | 25.685 | 36.926 | 14.983 | 1.00 | 35.19 | O | |
| ATOM | 2824 | O | HOH | W 124 | 5.593 | 17.615 | 20.205 | 1.00 | 22.22 | O | |
| ATOM | 2825 | O | HOH | W 125 | −3.621 | 36.444 | 16.059 | 1.00 | 38.53 | O | |
| ATOM | 2826 | O | HOH | W 126 | −6.822 | 38.370 | 14.785 | 1.00 | 35.77 | O | |
| ATOM | 2827 | O | HOH | W 127 | 8.658 | 28.063 | 15.129 | 1.00 | 32.14 | O | |
| ATOM | 2828 | O | HOH | W 128 | −4.302 | 30.987 | 36.514 | 1.00 | 38.06 | O | |
| ATOM | 2829 | O | HOH | W 129 | −1.650 | 29.068 | 38.757 | 1.00 | 30.65 | O | |
| TER | 2830 | | HOH | W 129 | | | | | | | |
| END | | | | | | | | | | | |

TABLE 4
REMARK coordinates from restrained individual B-factor refinement
REMARK refinement resolution: 500.0 − 2.05 A
REMARK starting r = 0.2078 free_r = 0.2459
REMARK final r = 0.2076 free_r = 0.2466
REMARK B rmsd for bonded mainchain atoms = 1.338 target = 1.5
REMARK B rmsd for bonded sidechain atoms = 2.017 target = 2.0
REMARK B rmsd for angle mainchain atoms = 2.137 target = 2.0
REMARK B rmsd for angle sidechain atoms = 3.077 target = 2.5
REMARK wa = 1.98578
REMARK rweight = 0.12477
REMARK target = mlf · steps = 30
REMARK sg = C2 a = 116.043 b = 59.291 c = 49.629
alpha = 90 beta = 98.768 gamma = 90
REMARK parameter file 1 CNX_TOPPAR:protein_rep.param
REMARK parameter file 2 CNX_TOPPAR:water_rep.param
REMARK parameter file 3 CNX_TOPPAR:ion.param
REMARK molecular structure file: generate.psf
REMARK input coordinates: minimize.pdb
REMARK reflection file = ../data/x-ray_data.cv
REMARK ncs = none
REMARK B-correction resolution: 6.0 − 2.05
REMARK initial B-factor correction applied to fobs:
REMARK B11 = 0.091 B22 = −2.051 B33 = 1.959
REMARK B12 = 0.000 B13 = 0.271 B23 = 0.000
REMARK B-factor correction applied to coordinate array B: −0.022
REMARK bulk solvent: (Mask) density level = 0.368883 e/A\hat{ }3, B-factor = 52.2583 A\hat{ }2
REMARK reflections with |Fobs|/sigma_F < 0.0 rejected
REMARK reflections with |Fobs| > 10000 * rms (Fobs) rejected

| | | |
|---|---|---|
| REMARK theoretical total number of refl. in resol. range: | 21048 | (100.0%) |
| REMARK number of unobserved reflections (no entry or \|F\| = 0): | 1611 | (7.7%) |
| REMARK number of reflections rejected: | 0 | (0.0%) |
| REMARK total number of reflections used: | 19437 | (92.3%) |
| REMARK number of reflections in working set: | 18494 | (87.9%) |
| REMARK number of reflections in test set: | 943 | (4.5%) |

REMARK FILENAME = “acps_native 2.pdb”:

created by user: rx83663

REMARK Written by CNX VERSION: 2000

| | | | | | | | | | | |
|---|---|---|---|---|---|---|---|---|---|---|
| ATOM | 1 | CB | MET | A1003 | 36.109 | 5.413 | 19.351 | 1.00 | 32.48 | C |
| ATOM | 2 | CG | MET | A1003 | 35.473 | 5.856 | 20.649 | 1.00 | 37.79 | C |
| ATOM | 3 | SD | MET | A1003 | 36.607 | 6.848 | 21.649 | 1.00 | 45.89 | S |
| ATOM | 4 | CE | MET | A1003 | 36.791 | 8.314 | 20.599 | 1.00 | 43.10 | C |
| ATOM | 5 | C | MET | A1003 | 33.900 | 5.073 | 18.225 | 1.00 | 27.65 | C |
| ATOM | 6 | O | MET | A1003 | 33.795 | 6.027 | 17.463 | 1.00 | 25.93 | O |
| ATOM | 7 | N | MET | A1003 | 35.971 | 4.133 | 17.240 | 1.00 | 28.87 | N |
| ATOM | 8 | CA | MET | A1003 | 35.262 | 4.452 | 18.515 | 1.00 | 29.37 | C |
| ATOM | 9 | N | ILE | A1004 | 32.860 | 4.513 | 18.832 | 1.00 | 27.12 | N |
| ATOM | 10 | CA | ILE | A1004 | 31.500 | 5.005 | 18.649 | 1.00 | 25.95 | C |
| ATOM | 11 | CB | ILE | A1004 | 30.463 | 3.928 | 19.020 | 1.00 | 25.43 | C |
| ATOM | 12 | CG2 | ILE | A1004 | 29.048 | 4.516 | 18.954 | 1.00 | 24.89 | C |
| ATOM | 13 | CG1 | ILE | A1004 | 30.604 | 2.730 | 18.076 | 1.00 | 24.55 | C |
| ATOM | 14 | CD1 | ILE | A1004 | 29.619 | 1.616 | 18.347 | 1.00 | 26.14 | C |
| ATOM | 15 | C | ILE | A1004 | 31.289 | 6.214 | 19.546 | 1.00 | 27.18 | C |
| ATOM | 16 | O | ILE | A1004 | 31.700 | 6.212 | 20.703 | 1.00 | 27.39 | O |
| ATOM | 17 | N | VAL | A1005 | 30.655 | 7.251 | 19.018 | 1.00 | 24.87 | N |
| ATOM | 18 | CA | VAL | A1005 | 30.420 | 8.439 | 19.818 | 1.00 | 25.40 | C |
| ATOM | 19 | CB | VAL | A1005 | 31.326 | 9.589 | 19.346 | 1.00 | 26.42 | C |
| ATOM | 20 | CG1 | VAL | A1005 | 32.781 | 9.240 | 19.648 | 1.00 | 26.20 | C |
| ATOM | 21 | CG2 | VAL | A1005 | 31.153 | 9.818 | 17.860 | 1.00 | 27.65 | C |
| ATOM | 22 | C | VAL | A1005 | 28.955 | 8.857 | 19.794 | 1.00 | 24.30 | C |
| ATOM | 23 | O | VAL | A1005 | 28.592 | 9.964 | 20.195 | 1.00 | 23.88 | O |
| ATOM | 24 | N | GLY | A1006 | 28.102 | 7.952 | 19.331 | 1.00 | 23.00 | N |
| ATOM | 25 | CA | GLY | A1006 | 26.687 | 8.261 | 19.290 | 1.00 | 19.45 | C |
| ATOM | 26 | C | GLY | A1006 | 25.887 | 7.300 | 18.443 | 1.00 | 18.59 | C |
| ATOM | 27 | O | GLY | A1006 | 26.396 | 6.730 | 17.480 | 1.00 | 16.47 | O |
| ATOM | 28 | N | HIS | A1007 | 24.621 | 7.130 | 18.809 | 1.00 | 16.39 | N |
| ATOM | 29 | CA | HIS | A1007 | 23.720 | 6.249 | 18.092 | 1.00 | 15.38 | C |
| ATOM | 30 | CB | HIS | A1007 | 23.825 | 4.818 | 18.636 | 1.00 | 17.15 | C |
| ATOM | 31 | CG | HIS | A1007 | 22.877 | 3.853 | 17.993 | 1.00 | 18.41 | C |
| ATOM | 32 | CD2 | HIS | A1007 | 22.075 | 2.902 | 18.528 | 1.00 | 17.85 | C |
| ATOM | 33 | ND1 | HIS | A1007 | 22.690 | 3.789 | 16.628 | 1.00 | 17.93 | N |
| ATOM | 34 | CE1 | HIS | A1007 | 21.811 | 2.841 | 16.351 | 1.00 | 19.72 | C |
| ATOM | 35 | NE2 | HIS | A1007 | 21.423 | 2.288 | 17.486 | 1.00 | 18.78 | N |
| ATOM | 36 | C | HIS | A1007 | 22.302 | 6.768 | 18.262 | 1.00 | 16.54 | C |
| ATOM | 37 | O | HIS | A1007 | 21.883 | 7.113 | 19.364 | 1.00 | 15.23 | O |
| ATOM | 38 | N | GLY | A1008 | 21.568 | 6.844 | 17.162 | 1.00 | 13.65 | N |
| ATOM | 39 | CA | GLY | A1008 | 20.204 | 7.303 | 17.250 | 1.00 | 14.59 | C |
| ATOM | 40 | C | GLY | A1008 | 19.364 | 6.591 | 16.220 | 1.00 | 16.81 | C |
| ATOM | 41 | O | GLY | A1008 | 19.864 | 6.208 | 15.154 | 1.00 | 14.95 | O |
| ATOM | 42 | N | ILE | A1009 | 18.089 | 6.394 | 16.538 | 1.00 | 16.05 | N |
| ATOM | 43 | CA | ILE | A1009 | 17.186 | 5.750 | 15.603 | 1.00 | 16.53 | C |
| ATOM | 44 | CB | ILE | A1009 | 16.841 | 4.297 | 16.012 | 1.00 | 16.37 | C |
| ATOM | 45 | CG2 | ILE | A1009 | 18.087 | 3.436 | 15.974 | 1.00 | 15.97 | C |
| ATOM | 46 | CG1 | ILE | A1009 | 16.168 | 4.284 | 17.393 | 1.00 | 14.54 | C |
| ATOM | 47 | CD1 | ILE | A1009 | 15.697 | 2.907 | 17.831 | 1.00 | 15.26 | C |
| ATOM | 48 | C | ILE | A1009 | 15.898 | 6.541 | 15.578 | 1.00 | 18.21 | C |
| ATOM | 49 | O | ILE | A1009 | 15.681 | 7.423 | 16.408 | 1.00 | 18.91 | O |
| ATOM | 50 | N | ASP | A1010 | 15.041 | 6.218 | 14.623 | 1.00 | 19.48 | N |
| ATOM | 51 | CA | ASP | A1010 | 13.759 | 6.880 | 14.520 | 1.00 | 20.47 | C |
| ATOM | 52 | CB | ASP | A1010 | 13.930 | 8.295 | 13.969 | 1.00 | 21.20 | C |
| ATOM | 53 | CG | ASP | A1010 | 12.614 | 9.013 | 13.813 | 1.00 | 24.68 | C |
| ATOM | 54 | OD1 | ASP | A1010 | 12.002 | 8.901 | 12.725 | 1.00 | 25.58 | O |
| ATOM | 55 | OD2 | ASP | A1010 | 12.182 | 9.675 | 14.785 | 1.00 | 21.51 | O |
| ATOM | 56 | C | ASP | A1010 | 12.822 | 6.094 | 13.630 | 1.00 | 22.13 | C |
| ATOM | 57 | O | ASP | A1010 | 13.249 | 5.485 | 12.641 | 1.00 | 21.63 | O |
| ATOM | 58 | N | ILE | A1011 | 11.548 | 6.074 | 14.011 | 1.00 | 20.69 | N |
| ATOM | 59 | CA | ILE | A1011 | 10.525 | 5.403 | 13.226 | 1.00 | 22.73 | C |
| ATOM | 60 | CB | ILE | A1011 | 10.019 | 4.103 | 13.903 | 1.00 | 22.88 | C |
| ATOM | 61 | CG2 | ILE | A1011 | 9.505 | 4.398 | 15.320 | 1.00 | 23.25 | C |
| ATOM | 62 | CG1 | ILE | A1011 | 8.922 | 3.476 | 13.033 | 1.00 | 24.40 | C |
| ATOM | 63 | CD1 | ILE | A1011 | 8.496 | 2.096 | 13.466 | 1.00 | 25.25 | C |
| ATOM | 64 | C | ILE | A1011 | 9.394 | 6.412 | 13.094 | 1.00 | 23.63 | C |
| ATOM | 65 | O | ILE | A1011 | 9.016 | 7.061 | 14.071 | 1.00 | 23.36 | O |
| ATOM | 66 | N | GLU | A1012 | 8.869 | 6.549 | 11.882 | 1.00 | 25.11 | N |
| ATOM | 67 | CA | GLU | A1012 | 7.810 | 7.514 | 11.602 | 1.00 | 24.91 | C |
| ATOM | 68 | CB | GLU | A1012 | 8.421 | 8.737 | 10.916 | 1.00 | 25.40 | C |
| ATOM | 69 | CG | GLU | A1012 | 7.413 | 9.773 | 10.433 | 1.00 | 29.98 | C |
| ATOM | 70 | CD | GLU | A1012 | 6.857 | 10.631 | 11.552 | 1.00 | 32.43 | C |
| ATOM | 71 | OE1 | GLU | A1012 | 5.954 | 11.447 | 11.274 | 1.00 | 36.73 | O |
| ATOM | 72 | OE2 | GLU | A1012 | 7.319 | 10.497 | 12.704 | 1.00 | 35.01 | O |
| ATOM | 73 | C | GLU | A1012 | 6.705 | 6.952 | 10.716 | 1.00 | 24.25 | C |
| ATOM | 74 | O | GLU | A1012 | 6.975 | 6.186 | 9.789 | 1.00 | 22.71 | O |
| ATOM | 75 | N | GLU | A1013 | 5.462 | 7.331 | 11.006 | 1.00 | 24.75 | N |
| ATOM | 76 | CA | GLU | A1013 | 4.326 | 6.895 | 10.195 | 1.00 | 25.26 | C |
| ATOM | 77 | CB | GLU | A1013 | 3.006 | 7.010 | 10.956 | 1.00 | 27.28 | C |
| ATOM | 78 | CG | GLU | A1013 | 2.782 | 6.015 | 12.056 | 1.00 | 32.89 | C |
| ATOM | 79 | CD | GLU | A1013 | 1.337 | 6.025 | 12.512 | 1.00 | 36.18 | C |
| ATOM | 80 | OE1 | GLU | A1013 | 0.451 | 5.792 | 11.661 | 1.00 | 38.66 | O |
| ATOM | 81 | OE2 | GLU | A1013 | 1.083 | 6.270 | 13.709 | 1.00 | 39.09 | O |
| ATOM | 82 | C | GLU | A1013 | 4.253 | 7.848 | 9.013 | 1.00 | 24.28 | C |
| ATOM | 83 | O | GLU | A1013 | 4.254 | 9.062 | 9.197 | 1.00 | 24.07 | O |
| ATOM | 84 | N | LEU | A1014 | 4.179 | 7.308 | 7.805 | 1.00 | 23.86 | N |
| ATOM | 85 | CA | LEU | A1014 | 4.103 | 8.149 | 6.615 | 1.00 | 24.45 | C |
| ATOM | 86 | CB | LEU | A1014 | 4.150 | 7.274 | 5.362 | 1.00 | 25.54 | C |
| ATOM | 87 | CG | LEU | A1014 | 5.402 | 6.397 | 5.287 | 1.00 | 28.12 | C |
| ATOM | 88 | CD1 | LEU | A1014 | 5.266 | 5.386 | 4.162 | 1.00 | 28.37 | C |
| ATOM | 89 | CD2 | LEU | A1014 | 6.627 | 7.284 | 5.082 | 1.00 | 29.49 | C |
| ATOM | 90 | C | LEU | A1014 | 2.838 | 9.010 | 6.611 | 1.00 | 23.06 | C |
| ATOM | 91 | O | LEU | A1014 | 2.838 | 10.126 | 6.083 | 1.00 | 22.31 | O |
| ATOM | 92 | N | ALA | A1015 | 1.768 | 8.490 | 7.210 | 1.00 | 22.30 | N |
| ATOM | 93 | CA | ALA | A1015 | 0.489 | 9.202 | 7.284 | 1.00 | 21.59 | C |
| ATOM | 94 | CB | ALA | A1015 | −0.529 | 8.372 | 8.068 | 1.00 | 20.65 | C |
| ATOM | 95 | C | ALA | A1015 | 0.664 | 10.568 | 7.938 | 1.00 | 21.21 | C |
| ATOM | 96 | O | ALA | A1015 | 0.101 | 11.562 | 7.483 | 1.00 | 19.58 | O |
| ATOM | 97 | N | SER | A1016 | 1.442 | 10.605 | 9.017 | 1.00 | 21.38 | N |
| ATOM | 98 | CA | SER | A1016 | 1.711 | 11.848 | 9.728 | 1.00 | 22.18 | C |
| ATOM | 99 | CB | SER | A1016 | 2.587 | 11.569 | 10.951 | 1.00 | 22.67 | C |
| ATOM | 100 | OG | SER | A1016 | 2.034 | 10.523 | 11.733 | 1.00 | 29.64 | O |
| ATOM | 101 | C | SER | A1016 | 2.428 | 12.825 | 8.800 | 1.00 | 22.03 | C |
| ATOM | 102 | O | SER | A1016 | 2.134 | 14.020 | 8.786 | 1.00 | 24.58 | O |
| ATOM | 103 | N | ILE | A1017 | 3.382 | 12.319 | 8.027 | 1.00 | 21.56 | N |
| ATOM | 104 | CA | ILE | A1017 | 4.112 | 13.177 | 7.104 | 1.00 | 22.10 | C |
| ATOM | 105 | CB | ILE | A1017 | 5.336 | 12.454 | 6.523 | 1.00 | 22.24 | C |
| ATOM | 106 | CG2 | ILE | A1017 | 5.975 | 13.312 | 5.432 | 1.00 | 21.89 | C |
| ATOM | 107 | CG1 | ILE | A1017 | 6.342 | 12.174 | 7.641 | 1.00 | 22.58 | C |
| ATOM | 108 | CD1 | ILE | A1017 | 6.904 | 13.440 | 8.287 | 1.00 | 24.27 | C |
| ATOM | 109 | C | ILE | A1017 | 3.207 | 13.633 | 5.957 | 1.00 | 22.93 | C |
| ATOM | 110 | O | ILE | A1017 | 3.244 | 14.793 | 5.549 | 1.00 | 23.54 | O |
| ATOM | 111 | N | GLU | A1018 | 2.388 | 12.726 | 5.440 | 1.00 | 23.95 | N |
| ATOM | 112 | CA | GLU | A1018 | 1.494 | 13.096 | 4.348 | 1.00 | 26.57 | C |
| ATOM | 113 | CB | GLU | A1018 | 0.794 | 11.862 | 3.775 | 1.00 | 29.38 | C |
| ATOM | 114 | CG | GLU | A1018 | −0.244 | 12.197 | 2.709 | 1.00 | 33.05 | C |
| ATOM | 115 | CD | GLU | A1018 | −0.655 | 10.987 | 1.900 | 1.00 | 36.21 | C |
| ATOM | 116 | OE1 | GLU | A1018 | −0.803 | 9.897 | 2.495 | 1.00 | 38.49 | O |
| ATOM | 117 | OE2 | GLU | A1018 | −0.840 | 11.131 | 0.672 | 1.00 | 38.02 | O |
| ATOM | 118 | C | GLU | A1018 | 0.466 | 14.116 | 4.819 | 1.00 | 25.75 | C |
| ATOM | 119 | O | GLU | A1018 | 0.115 | 15.032 | 4.082 | 1.00 | 25.64 | O |
| ATOM | 120 | N | SER | A1019 | −0.011 | 13.960 | 6.052 | 1.00 | 27.25 | N |
| ATOM | 121 | CA | SER | A1019 | −0.980 | 14.893 | 6.615 | 1.00 | 27.84 | C |
| ATOM | 122 | CB | SER | A1019 | −1.436 | 14.425 | 8.000 | 1.00 | 28.92 | C |
| ATOM | 123 | OG | SER | A1019 | −2.107 | 13.176 | 7.929 | 1.00 | 31.15 | O |
| ATOM | 124 | C | SER | A1019 | −0.338 | 16.275 | 6.734 | 1.00 | 28.62 | C |
| ATOM | 125 | O | SER | A1019 | −0.956 | 17.289 | 6.400 | 1.00 | 27.65 | O |
| ATOM | 126 | N | ALA | A1020 | 0.907 | 16.302 | 7.209 | 1.00 | 28.27 | N |
| ATOM | 127 | CA | ALA | A1020 | 1.650 | 17.545 | 7.374 | 1.00 | 30.56 | C |
| ATOM | 128 | CB | ALA | A1020 | 3.020 | 17.267 | 8.005 | 1.00 | 30.65 | C |
| ATOM | 129 | C | ALA | A1020 | 1.823 | 18.267 | 6.045 | 1.00 | 31.87 | C |
| ATOM | 130 | O | ALA | A1020 | 1.686 | 19.487 | 5.976 | 1.00 | 32.82 | O |
| ATOM | 131 | N | VAL | A1021 | 2.127 | 17.513 | 4.992 | 1.00 | 33.76 | N |
| ATOM | 132 | CA | VAL | A1021 | 2.304 | 18.099 | 3.666 | 1.00 | 34.05 | C |
| ATOM | 133 | CB | VAL | A1021 | 2.830 | 17.057 | 2.656 | 1.00 | 34.30 | C |
| ATOM | 134 | CG1 | VAL | A1021 | 2.878 | 17.660 | 1.263 | 1.00 | 31.58 | C |
| ATOM | 135 | CG2 | VAL | A1021 | 4.213 | 16.577 | 3.073 | 1.00 | 33.12 | C |
| ATOM | 136 | C | VAL | A1021 | 0.969 | 18.635 | 3.150 | 1.00 | 35.65 | C |
| ATOM | 137 | O | VAL | A1021 | 0.891 | 19.754 | 2.643 | 1.00 | 34.17 | O |
| ATOM | 138 | N | THR | A1022 | −0.081 | 17.829 | 3.294 | 1.00 | 36.17 | N |
| ATOM | 139 | CA | THR | A1022 | −1.418 | 18.209 | 2.842 | 1.00 | 37.29 | C |
| ATOM | 140 | CB | THR | A1022 | −2.433 | 17.074 | 3.096 | 1.00 | 35.53 | C |
| ATOM | 141 | OG1 | THR | A1022 | −2.107 | 15.955 | 2.265 | 1.00 | 34.37 | O |
| ATOM | 142 | CG2 | THR | A1022 | −3.845 | 17.530 | 2.776 | 1.00 | 35.58 | C |
| ATOM | 143 | C | THR | A1022 | −1.933 | 19.482 | 3.504 | 1.00 | 39.32 | C |
| ATOM | 144 | O | THR | A1022 | −2.672 | 20.250 | 2.885 | 1.00 | 38.90 | O |
| ATOM | 145 | N | ARG | A1023 | −1.544 | 19.702 | 4.758 | 1.00 | 42.46 | N |
| ATOM | 146 | CA | ARG | A1023 | −1.977 | 20.885 | 5.496 | 1.00 | 46.07 | C |
| ATOM | 147 | CB | ARG | A1023 | −1.684 | 20.727 | 6.991 | 1.00 | 48.22 | C |
| ATOM | 148 | CG | ARG | A1023 | −2.327 | 19.534 | 7.664 | 1.00 | 51.97 | C |
| ATOM | 149 | CD | ARG | A1023 | −1.900 | 19.477 | 9.125 | 1.00 | 55.95 | C |
| ATOM | 150 | NE | ARG | A1023 | −2.234 | 18.208 | 9.770 | 1.00 | 59.02 | N |
| ATOM | 151 | CZ | ARG | A1023 | −1.958 | 17.922 | 11.039 | 1.00 | 61.01 | C |
| ATOM | 152 | NH1 | ARG | A1023 | −1.344 | 18.818 | 11.804 | 1.00 | 61.80 | N |
| ATOM | 153 | NH2 | ARG | A1023 | −2.292 | 16.741 | 11.546 | 1.00 | 61.79 | N |
| ATOM | 154 | C | ARG | A1023 | −1.275 | 22.147 | 4.999 | 1.00 | 47.81 | C |
| ATOM | 155 | O | ARG | A1023 | −1.801 | 22.888 | 4.166 | 1.00 | 47.51 | O |
| ATOM | 156 | N | HIS | A1024 | −0.077 | 22.381 | 5.526 | 1.00 | 49.48 | N |
| ATOM | 157 | CA | HIS | A1024 | 0.714 | 23.553 | 5.171 | 1.00 | 49.99 | C |
| ATOM | 158 | CB | HIS | A1024 | 1.238 | 24.226 | 6.441 | 1.00 | 51.40 | C |
| ATOM | 159 | CG | HIS | A1024 | 1.367 | 23.295 | 7.608 | 1.00 | 53.33 | C |
| ATOM | 160 | CD2 | HIS | A1024 | 2.447 | 22.670 | 8.136 | 1.00 | 53.80 | C |
| ATOM | 161 | ND1 | HIS | A1024 | 0.284 | 22.885 | 8.357 | 1.00 | 54.21 | N |
| ATOM | 162 | CE1 | HIS | A1024 | 0.691 | 22.048 | 9.295 | 1.00 | 54.57 | C |
| ATOM | 163 | NE2 | HIS | A1024 | 1.999 | 21.900 | 9.183 | 1.00 | 54.28 | N |
| ATOM | 164 | C | HIS | A1024 | 1.877 | 23.218 | 4.249 | 1.00 | 49.79 | C |
| ATOM | 165 | O | HIS | A1024 | 2.655 | 22.296 | 4.513 | 1.00 | 49.42 | O |
| ATOM | 166 | N | GLU | A1025 | 1.987 | 23.983 | 3.167 | 1.00 | 49.05 | N |
| ATOM | 167 | CA | GLU | A1025 | 3.043 | 23.796 | 2.179 | 1.00 | 48.04 | C |
| ATOM | 168 | CB | GLU | A1025 | 2.813 | 24.722 | 0.984 | 1.00 | 50.62 | C |
| ATOM | 169 | CG | GLU | A1025 | 3.876 | 24.605 | −0.092 | 1.00 | 54.70 | C |
| ATOM | 170 | CD | GLU | A1025 | 4.200 | 25.938 | −0.735 | 1.00 | 56.22 | C |
| ATOM | 171 | OE1 | GLU | A1025 | 3.295 | 26.547 | −1.343 | 1.00 | 58.24 | O |
| ATOM | 172 | OE2 | GLU | A1025 | 5.365 | 26.377 | −0.627 | 1.00 | 58.15 | O |
| ATOM | 173 | C | GLU | A1025 | 4.418 | 24.081 | 2.768 | 1.00 | 45.80 | C |
| ATOM | 174 | O | GLU | A1025 | 5.433 | 23.604 | 2.258 | 1.00 | 46.85 | O |
| ATOM | 175 | N | GLY | A1026 | 4.447 | 24.859 | 3.845 | 1.00 | 43.70 | N |
| ATOM | 176 | CA | GLY | A1026 | 5.709 | 25.202 | 4.474 | 1.00 | 39.18 | C |
| ATOM | 177 | C | GLY | A1026 | 6.425 | 24.069 | 5.192 | 1.00 | 37.08 | C |
| ATOM | 178 | O | GLY | A1026 | 7.572 | 24.235 | 5.608 | 1.00 | 36.56 | O |
| ATOM | 179 | N | PHE | A1027 | 5.771 | 22.920 | 5.337 | 1.00 | 33.78 | N |
| ATOM | 180 | CA | PHE | A1027 | 6.390 | 21.791 | 6.028 | 1.00 | 31.60 | C |
| ATOM | 181 | CB | PHE | A1027 | 5.397 | 20.637 | 6.179 | 1.00 | 30.22 | C |
| ATOM | 182 | CG | PHE | A1027 | 5.951 | 19.469 | 6.951 | 1.00 | 30.68 | C |
| ATOM | 183 | CD1 | PHE | A1027 | 6.224 | 19.586 | 8.312 | 1.00 | 29.31 | C |
| ATOM | 184 | CD2 | PHE | A1027 | 6.230 | 18.266 | 6.313 | 1.00 | 29.88 | C |
| ATOM | 185 | CE1 | PHE | A1027 | 6.768 | 18.523 | 9.027 | 1.00 | 31.21 | C |
| ATOM | 186 | CE2 | PHE | A1027 | 6.775 | 17.194 | 7.020 | 1.00 | 30.83 | C |
| ATOM | 187 | CZ | PHE | A1027 | 7.045 | 17.326 | 8.383 | 1.00 | 29.69 | C |
| ATOM | 188 | C | PHE | A1027 | 7.653 | 21.266 | 5.345 | 1.00 | 29.93 | C |
| ATOM | 189 | O | PHE | A1027 | 8.741 | 21.329 | 5.908 | 1.00 | 30.55 | O |
| ATOM | 190 | N | ALA | A1028 | 7.504 | 20.739 | 4.135 | 1.00 | 29.11 | N |
| ATOM | 191 | CA | ALA | A1028 | 8.637 | 20.196 | 3.400 | 1.00 | 28.78 | C |
| ATOM | 192 | CB | ALA | A1028 | 8.213 | 19.836 | 1.986 | 1.00 | 28.60 | C |
| ATOM | 193 | C | ALA | A1028 | 9.779 | 21.204 | 3.371 | 1.00 | 29.38 | C |
| ATOM | 194 | O | ALA | A1028 | 10.940 | 20.850 | 3.579 | 1.00 | 28.07 | O |
| ATOM | 195 | N | LYS | A1029 | 9.424 | 22.463 | 3.127 | 1.00 | 30.57 | N |
| ATOM | 196 | CA | LYS | A1029 | 10.372 | 23.574 | 3.062 | 1.00 | 32.15 | C |
| ATOM | 197 | CB | LYS | A1029 | 9.608 | 24.867 | 2.765 | 1.00 | 34.73 | C |
| ATOM | 198 | CG | LYS | A1029 | 10.076 | 25.630 | 1.535 | 1.00 | 38.51 | C |
| ATOM | 199 | CD | LYS | A1029 | 11.365 | 26.404 | 1.799 | 1.00 | 41.30 | C |
| ATOM | 200 | CE | LYS | A1029 | 11.653 | 27.390 | 0.669 | 1.00 | 41.77 | C |
| ATOM | 201 | NZ | LYS | A1029 | 12.865 | 28.214 | 0.927 | 1.00 | 43.68 | N |
| ATOM | 202 | C | LYS | A1029 | 11.173 | 23.729 | 4.357 | 1.00 | 31.51 | C |
| ATOM | 203 | O | LYS | A1029 | 12.362 | 24.043 | 4.326 | 1.00 | 31.47 | O |
| ATOM | 204 | N | ARG | A1030 | 10.521 | 23.512 | 5.495 | 1.00 | 31.52 | N |
| ATOM | 205 | CA | ARG | A1030 | 11.195 | 23.626 | 6.787 | 1.00 | 32.17 | C |
| ATOM | 206 | CB | ARG | A1030 | 10.188 | 23.625 | 7.949 | 1.00 | 34.49 | C |
| ATOM | 207 | CG | ARG | A1030 | 9.287 | 24.838 | 8.058 | 1.00 | 38.44 | C |
| ATOM | 208 | CD | ARG | A1030 | 8.775 | 25.030 | 9.497 | 1.00 | 41.54 | C |
| ATOM | 209 | NE | ARG | A1030 | 8.304 | 23.796 | 10.134 | 1.00 | 44.99 | N |
| ATOM | 210 | CZ | ARG | A1030 | 9.084 | 22.932 | 10.786 | 1.00 | 47.20 | C |
| ATOM | 211 | NH1 | ARG | A1030 | 10.388 | 23.157 | 10.898 | 1.00 | 48.13 | N |
| ATOM | 212 | NH2 | ARG | A1030 | 8.562 | 21.841 | 11.337 | 1.00 | 47.12 | N |
| ATOM | 213 | C | ARG | A1030 | 12.145 | 22.460 | 7.013 | 1.00 | 30.09 | C |
| ATOM | 214 | O | ARG | A1030 | 13.248 | 22.631 | 7.530 | 1.00 | 29.93 | O |
| ATOM | 215 | N | VAL | A1031 | 11.696 | 21.272 | 6.628 | 1.00 | 27.81 | N |
| ATOM | 216 | CA | VAL | A1031 | 12.460 | 20.050 | 6.819 | 1.00 | 25.50 | C |
| ATOM | 217 | CB | VAL | A1031 | 11.531 | 18.806 | 6.723 | 1.00 | 24.73 | C |
| ATOM | 218 | CG1 | VAL | A1031 | 12.355 | 17.518 | 6.871 | 1.00 | 24.35 | C |
| ATOM | 219 | CG2 | VAL | A1031 | 10.465 | 18.874 | 7.798 | 1.00 | 23.33 | C |
| ATOM | 220 | C | VAL | A1031 | 13.621 | 19.835 | 5.861 | 1.00 | 25.01 | C |
| ATOM | 221 | O | VAL | A1031 | 14.664 | 19.307 | 6.252 | 1.00 | 25.89 | O |
| ATOM | 222 | N | LEU | A1032 | 13.451 | 20.253 | 4.617 | 1.00 | 24.38 | N |
| ATOM | 223 | CA | LEU | A1032 | 14.472 | 20.027 | 3.604 | 1.00 | 25.25 | C |
| ATOM | 224 | CB | LEU | A1032 | 13.818 | 19.409 | 2.370 | 1.00 | 25.23 | C |
| ATOM | 225 | CG | LEU | A1032 | 12.870 | 18.230 | 2.597 | 1.00 | 23.47 | C |
| ATOM | 226 | CD1 | LEU | A1032 | 12.275 | 17.806 | 1.266 | 1.00 | 23.54 | C |
| ATOM | 227 | CD2 | LEU | A1032 | 13.624 | 17.072 | 3.239 | 1.00 | 23.54 | C |
| ATOM | 228 | C | LEU | A1032 | 15.271 | 21.243 | 3.169 | 1.00 | 25.67 | C |
| ATOM | 229 | O | LEU | A1032 | 14.778 | 22.371 | 3.184 | 1.00 | 25.62 | O |
| ATOM | 230 | N | THR | A1033 | 16.516 | 20.997 | 2.776 | 1.00 | 26.32 | N |
| ATOM | 231 | CA | THR | A1033 | 17.387 | 22.057 | 2.289 | 1.00 | 27.40 | C |
| ATOM | 232 | CB | THR | A1033 | 18.862 | 21.628 | 2.283 | 1.00 | 27.76 | C |
| ATOM | 233 | OG1 | THR | A1033 | 19.015 | 20.490 | 1.425 | 1.00 | 26.35 | O |
| ATOM | 234 | CG2 | THR | A1033 | 19.331 | 21.271 | 3.691 | 1.00 | 28.10 | C |
| ATOM | 235 | C | THR | A1033 | 16.963 | 22.275 | 0.844 | 1.00 | 27.65 | C |
| ATOM | 236 | O | THR | A1033 | 16.071 | 21.588 | 0.350 | 1.00 | 27.78 | O |
| ATOM | 237 | N | ALA | A1034 | 17.612 | 23.215 | 0.166 | 1.00 | 27.93 | N |
| ATOM | 238 | CA | ALA | A1034 | 17.294 | 23.502 | −1.225 | 1.00 | 28.06 | C |
| ATOM | 239 | CB | ALA | A1034 | 18.089 | 24.717 | −1.695 | 1.00 | 29.35 | C |
| ATOM | 240 | C | ALA | A1034 | 17.594 | 22.296 | −2.119 | 1.00 | 27.67 | C |
| ATOM | 241 | O | ALA | A1034 | 16.791 | 21.940 | −2.981 | 1.00 | 28.26 | O |
| ATOM | 242 | N | LEU | A1035 | 18.752 | 21.675 | −1.918 | 1.00 | 27.21 | N |
| ATOM | 243 | CA | LEU | A1035 | 19.131 | 20.510 | −2.711 | 1.00 | 29.21 | C |
| ATOM | 244 | CB | LEU | A1035 | 20.531 | 20.015 | −2.318 | 1.00 | 28.90 | C |
| ATOM | 245 | CG | LEU | A1035 | 21.743 | 20.903 | −2.634 | 1.00 | 32.48 | C |
| ATOM | 246 | CD1 | LEU | A1035 | 22.960 | 20.382 | −1.877 | 1.00 | 31.19 | C |
| ATOM | 247 | CD2 | LEU | A1035 | 22.017 | 20.924 | −4.137 | 1.00 | 32.53 | C |
| ATOM | 248 | C | LEU | A1035 | 18.111 | 19.388 | −2.514 | 1.00 | 27.97 | C |
| ATOM | 249 | O | LEU | A1035 | 17.674 | 18.758 | −3.475 | 1.00 | 28.70 | O |
| ATOM | 250 | N | GLU | A1036 | 17.731 | 19.143 | −1.267 | 1.00 | 27.27 | N |
| ATOM | 251 | CA | GLU | A1036 | 16.757 | 18.100 | −0.980 | 1.00 | 27.49 | C |
| ATOM | 252 | CB | GLU | A1036 | 16.599 | 17.938 | 0.532 | 1.00 | 27.41 | C |
| ATOM | 253 | CG | GLU | A1036 | 17.760 | 17.195 | 1.186 | 1.00 | 24.80 | C |
| ATOM | 254 | CD | GLU | A1036 | 17.720 | 17.252 | 2.703 | 1.00 | 26.22 | C |
| ATOM | 255 | OE1 | GLU | A1036 | 18.225 | 16.306 | 3.345 | 1.00 | 25.82 | O |
| ATOM | 256 | OE2 | GLU | A1036 | 17.201 | 18.248 | 3.256 | 1.00 | 26.57 | O |
| ATOM | 257 | C | GLU | A1036 | 15.413 | 18.416 | −1.634 | 1.00 | 27.55 | C |
| ATOM | 258 | O | GLU | A1036 | 14.740 | 17.522 | −2.138 | 1.00 | 25.83 | O |
| ATOM | 259 | N | MET | A1037 | 15.031 | 19.691 | −1.640 | 1.00 | 28.35 | N |
| ATOM | 260 | CA | MET | A1037 | 13.765 | 20.099 | −2.247 | 1.00 | 29.92 | C |
| ATOM | 261 | CB | MET | A1037 | 13.529 | 21.599 | −2.069 | 1.00 | 30.37 | C |
| ATOM | 262 | CG | MET | A1037 | 12.985 | 22.000 | −0.714 | 1.00 | 33.04 | C |
| ATOM | 263 | SD | MET | A1037 | 11.412 | 21.198 | −0.336 | 1.00 | 35.56 | S |
| ATOM | 264 | CE | MET | A1037 | 10.356 | 21.852 | −1.624 | 1.00 | 33.04 | C |
| ATOM | 265 | C | MET | A1037 | 13.758 | 19.779 | −3.731 | 1.00 | 31.19 | C |
| ATOM | 266 | O | MET | A1037 | 12.711 | 19.461 | −4.306 | 1.00 | 30.89 | O |
| ATOM | 267 | N | GLU | A1038 | 14.928 | 19.881 | −4.352 | 1.00 | 31.50 | N |
| ATOM | 268 | CA | GLU | A1038 | 15.049 | 19.600 | −5.774 | 1.00 | 33.14 | C |
| ATOM | 269 | CB | GLU | A1038 | 16.499 | 19.777 | −6.233 | 1.00 | 34.77 | C |
| ATOM | 270 | CG | GLU | A1038 | 17.005 | 21.208 | −6.093 | 1.00 | 39.68 | C |
| ATOM | 271 | CD | GLU | A1038 | 18.411 | 21.400 | −6.639 | 1.00 | 41.20 | C |
| ATOM | 272 | OE1 | GLU | A1038 | 18.981 | 22.492 | −6.434 | 1.00 | 44.15 | O |
| ATOM | 273 | OE2 | GLU | A1038 | 18.943 | 20.467 | −7.276 | 1.00 | 42.51 | O |
| ATOM | 274 | C | GLU | A1038 | 14.570 | 18.186 | −6.066 | 1.00 | 31.14 | C |
| ATOM | 275 | O | GLU | A1038 | 13.773 | 17.974 | −6.976 | 1.00 | 31.21 | O |
| ATOM | 276 | N | ARG | A1039 | 15.039 | 17.215 | −5.290 | 1.00 | 29.70 | N |
| ATOM | 277 | CA | ARG | A1039 | 14.606 | 15.844 | −5.521 | 1.00 | 28.01 | C |
| ATOM | 278 | CB | ARG | A1039 | 15.499 | 14.846 | −4.778 | 1.00 | 29.96 | C |
| ATOM | 279 | CG | ARG | A1039 | 15.061 | 13.406 | −5.005 | 1.00 | 30.43 | C |
| ATOM | 280 | CD | ARG | A1039 | 16.213 | 12.420 | −4.969 | 1.00 | 30.42 | C |
| ATOM | 281 | NE | ARG | A1039 | 15.723 | 11.046 | −5.085 | 1.00 | 31.83 | N |
| ATOM | 282 | CZ | ARG | A1039 | 15.203 | 10.509 | −6.188 | 1.00 | 32.36 | C |
| ATOM | 283 | NH1 | ARG | A1039 | 15.098 | 11.214 | −7.310 | 1.00 | 30.37 | N |
| ATOM | 284 | NH2 | ARG | A1039 | 14.764 | 9.258 | −6.161 | 1.00 | 32.43 | N |
| ATOM | 285 | C | ARG | A1039 | 13.158 | 15.671 | −5.086 | 1.00 | 26.14 | C |
| ATOM | 286 | O | ARG | A1039 | 12.367 | 15.046 | −5.785 | 1.00 | 25.25 | O |
| ATOM | 287 | N | PHE | A1040 | 12.808 | 16.233 | −3.934 | 1.00 | 24.86 | N |
| ATOM | 288 | CA | PHE | A1040 | 11.441 | 16.132 | −3.430 | 1.00 | 26.03 | C |
| ATOM | 289 | CB | PHE | A1040 | 11.273 | 17.007 | −2.178 | 1.00 | 25.44 | C |
| ATOM | 290 | CG | PHE | A1040 | 9.861 | 17.056 | −1.657 | 1.00 | 26.41 | C |
| ATOM | 291 | CD1 | PHE | A1040 | 9.298 | 15.953 | −1.027 | 1.00 | 25.83 | C |
| ATOM | 292 | CD2 | PHE | A1040 | 9.080 | 18.194 | −1.838 | 1.00 | 26.12 | C |
| ATOM | 293 | CE1 | PHE | A1040 | 7.977 | 15.977 | −0.585 | 1.00 | 24.52 | C |
| ATOM | 294 | CE2 | PHE | A1040 | 7.752 | 18.232 | −1.399 | 1.00 | 26.23 | C |
| ATOM | 295 | CZ | PHE | A1040 | 7.202 | 17.119 | −0.772 | 1.00 | 23.77 | C |
| ATOM | 296 | C | PHE | A1040 | 10.412 | 16.548 | −4.488 | 1.00 | 26.70 | C |
| ATOM | 297 | O | PHE | A1040 | 9.438 | 15.837 | −4.731 | 1.00 | 29.10 | O |
| ATOM | 298 | N | THR | A1041 | 10.635 | 17.691 | −5.126 | 1.00 | 28.25 | N |
| ATOM | 299 | CA | THR | A1041 | 9.705 | 18.195 | −6.136 | 1.00 | 30.17 | C |
| ATOM | 300 | CB | THR | A1041 | 9.935 | 19.700 | −6.391 | 1.00 | 31.11 | C |
| ATOM | 301 | OG1 | THR | A1041 | 11.255 | 19.911 | −6.904 | 1.00 | 32.37 | O |
| ATOM | 302 | CG2 | THR | A1041 | 9.781 | 20.478 | −5.099 | 1.00 | 29.66 | C |
| ATOM | 303 | C | THR | A1041 | 9.751 | 17.456 | −7.474 | 1.00 | 31.98 | C |
| ATOM | 304 | O | THR | A1041 | 8.863 | 17.627 | −8.311 | 1.00 | 32.84 | O |
| ATOM | 305 | N | SER | A1042 | 10.781 | 16.637 | −7.673 | 1.00 | 31.91 | N |
| ATOM | 306 | CA | SER | A1042 | 10.933 | 15.869 | −8.909 | 1.00 | 32.77 | C |
| ATOM | 307 | CB | SER | A1042 | 12.416 | 15.609 | −9.192 | 1.00 | 33.18 | C |
| ATOM | 308 | OG | SER | A1042 | 13.106 | 16.820 | −9.448 | 1.00 | 38.29 | O |
| ATOM | 309 | C | SER | A1042 | 10.215 | 14.529 | −8.831 | 1.00 | 32.24 | C |
| ATOM | 310 | O | SER | A1042 | 10.071 | 13.834 | −9.840 | 1.00 | 32.01 | O |
| ATOM | 311 | N | LEU | A1043 | 9.766 | 14.175 | −7.630 | 1.00 | 29.92 | N |
| ATOM | 312 | CA | LEU | A1043 | 9.101 | 12.901 | −7.394 | 1.00 | 28.48 | C |
| ATOM | 313 | CB | LEU | A1043 | 9.541 | 12.332 | −6.043 | 1.00 | 23.86 | C |
| ATOM | 314 | CG | LEU | A1043 | 11.050 | 12.097 | −5.928 | 1.00 | 22.34 | C |
| ATOM | 315 | CD1 | LEU | A1043 | 11.391 | 11.610 | −4.538 | 1.00 | 16.25 | C |
| ATOM | 316 | CD2 | LEU | A1043 | 11.493 | 11.073 | −6.977 | 1.00 | 20.12 | C |
| ATOM | 317 | C | LEU | A1043 | 7.587 | 12.967 | −7.454 | 1.00 | 30.04 | C |
| ATOM | 318 | O | LEU | A1043 | 6.995 | 14.045 | −7.415 | 1.00 | 29.66 | O |
| ATOM | 319 | N | LYS | A1044 | 6.970 | 11.793 | −7.524 | 1.00 | 30.79 | N |
| ATOM | 320 | CA | LYS | A1044 | 5.525 | 11.679 | −7.633 | 1.00 | 32.89 | C |
| ATOM | 321 | CB | LYS | A1044 | 5.167 | 11.102 | −9.008 | 1.00 | 36.71 | C |
| ATOM | 322 | CG | LYS | A1044 | 5.598 | 11.946 | −10.199 | 1.00 | 40.80 | C |
| ATOM | 323 | CD | LYS | A1044 | 4.583 | 13.041 | −10.500 | 1.00 | 45.23 | C |
| ATOM | 324 | CE | LYS | A1044 | 4.793 | 13.615 | −11.897 | 1.00 | 47.01 | C |
| ATOM | 325 | NZ | LYS | A1044 | 3.591 | 14.354 | −12.387 | 1.00 | 48.79 | N |
| ATOM | 326 | C | LYS | A1044 | 4.883 | 10.801 | −6.564 | 1.00 | 31.67 | C |
| ATOM | 327 | O | LYS | A1044 | 5.512 | 9.894 | −6.017 | 1.00 | 31.18 | O |
| ATOM | 328 | N | GLY | A1045 | 3.611 | 11.080 | −6.299 | 1.00 | 30.41 | N |
| ATOM | 329 | CA | GLY | A1045 | 2.834 | 10.321 | −5.337 | 1.00 | 31.09 | C |
| ATOM | 330 | C | GLY | A1045 | 3.518 | 9.841 | −4.074 | 1.00 | 31.50 | C |
| ATOM | 331 | O | GLY | A1045 | 4.098 | 10.631 | −3.329 | 1.00 | 32.31 | O |
| ATOM | 332 | N | ARG | A1046 | 3.435 | 8.534 | −3.841 | 1.00 | 31.79 | N |
| ATOM | 333 | CA | ARG | A1046 | 4.009 | 7.904 | −2.659 | 1.00 | 34.20 | C |
| ATOM | 334 | CB | ARG | A1046 | 3.791 | 6.386 | −2.717 | 1.00 | 37.24 | C |
| ATOM | 335 | CG | ARG | A1046 | 2.420 | 5.971 | −3.239 | 1.00 | 43.96 | C |
| ATOM | 336 | CD | ARG | A1046 | 2.301 | 4.457 | −3.386 | 1.00 | 48.00 | C |
| ATOM | 337 | NE | ARG | A1046 | 1.885 | 3.810 | −2.144 | 1.00 | 52.29 | N |
| ATOM | 338 | CZ | ARG | A1046 | 1.793 | 2.493 | −1.980 | 1.00 | 53.80 | C |
| ATOM | 339 | NH1 | ARG | A1046 | 1.399 | 1.995 | −0.815 | 1.00 | 54.77 | N |
| ATOM | 340 | NH2 | ARG | A1046 | 2.106 | 1.673 | −2.975 | 1.00 | 56.31 | N |
| ATOM | 341 | C | ARG | A1046 | 5.499 | 8.194 | −2.516 | 1.00 | 32.21 | C |
| ATOM | 342 | O | ARG | A1046 | 5.972 | 8.529 | −1.433 | 1.00 | 32.82 | O |
| ATOM | 343 | N | ARG | A1047 | 6.234 | 8.060 | −3.614 | 1.00 | 31.05 | N |
| ATOM | 344 | CA | ARG | A1047 | 7.670 | 8.292 | −3.605 | 1.00 | 30.41 | C |
| ATOM | 345 | CB | ARG | A1047 | 8.235 | 8.126 | −5.015 | 1.00 | 32.83 | C |
| ATOM | 346 | CG | ARG | A1047 | 9.252 | 7.013 | −5.123 | 1.00 | 37.44 | C |
| ATOM | 347 | CD | ARG | A1047 | 8.909 | 6.055 | −6.246 | 1.00 | 40.57 | C |
| ATOM | 348 | NE | ARG | A1047 | 9.734 | 4.854 | −6.183 | 1.00 | 45.77 | N |
| ATOM | 349 | CZ | ARG | A1047 | 9.635 | 3.835 | −7.028 | 1.00 | 47.39 | C |
| ATOM | 350 | NH1 | ARG | A1047 | 10.428 | 2.780 | −6.896 | 1.00 | 49.29 | N |
| ATOM | 351 | NH2 | ARG | A1047 | 8.743 | 3.873 | −8.008 | 1.00 | 48.96 | N |
| ATOM | 352 | C | ARG | A1047 | 8.040 | 9.668 | −3.062 | 1.00 | 28.90 | C |
| ATOM | 353 | O | ARG | A1047 | 9.006 | 9.808 | −2.307 | 1.00 | 27.20 | O |
| ATOM | 354 | N | GLN | A1048 | 7.268 | 10.680 | −3.444 | 1.00 | 26.75 | N |
| ATOM | 355 | CA | GLN | A1048 | 7.522 | 12.042 | −2.993 | 1.00 | 25.72 | C |
| ATOM | 356 | CB | GLN | A1048 | 6.552 | 13.014 | −3.679 | 1.00 | 26.20 | C |
| ATOM | 357 | CG | GLN | A1048 | 6.972 | 14.472 | −3.555 | 1.00 | 26.13 | C |
| ATOM | 358 | CD | GLN | A1048 | 6.169 | 15.400 | −4.449 | 1.00 | 25.31 | C |
| ATOM | 359 | OE1 | GLN | A1048 | 6.618 | 16.495 | −4.773 | 1.00 | 27.04 | O |
| ATOM | 360 | NE2 | GLN | A1048 | 4.977 | 14.970 | −4.843 | 1.00 | 24.57 | N |
| ATOM | 361 | C | GLN | A1048 | 7.390 | 12.162 | −1.475 | 1.00 | 23.82 | C |
| ATOM | 362 | O | GLN | A1048 | 8.225 | 12.783 | −0.819 | 1.00 | 23.93 | O |
| ATOM | 363 | N | ILE | A1049 | 6.340 | 11.568 | −0.919 | 1.00 | 23.76 | N |
| ATOM | 364 | CA | ILE | A1049 | 6.126 | 11.615 | 0.523 | 1.00 | 23.77 | C |
| ATOM | 365 | CB | ILE | A1049 | 4.733 | 11.084 | 0.900 | 1.00 | 25.38 | C |
| ATOM | 366 | CG2 | ILE | A1049 | 4.598 | 11.001 | 2.401 | 1.00 | 25.87 | C |
| ATOM | 367 | CG1 | ILE | A1049 | 3.657 | 11.999 | 0.315 | 1.00 | 26.78 | C |
| ATOM | 368 | CD1 | ILE | A1049 | 3.787 | 13.453 | 0.751 | 1.00 | 30.33 | C |
| ATOM | 369 | C | ILE | A1049 | 7.181 | 10.776 | 1.228 | 1.00 | 22.10 | C |
| ATOM | 370 | O | ILE | A1049 | 7.673 | 11.150 | 2.283 | 1.00 | 21.61 | O |
| ATOM | 371 | N | GLU | A1050 | 7.525 | 9.642 | 0.631 | 1.00 | 22.60 | N |
| ATOM | 372 | CA | GLU | A1050 | 8.532 | 8.755 | 1.200 | 1.00 | 23.30 | C |
| ATOM | 373 | CB | GLU | A1050 | 8.667 | 7.478 | 0.363 | 1.00 | 23.16 | C |
| ATOM | 374 | CG | GLU | A1050 | 9.889 | 6.659 | 0.739 | 1.00 | 28.58 | C |
| ATOM | 375 | CD | GLU | A1050 | 9.978 | 6.445 | 2.234 | 1.00 | 29.28 | C |
| ATOM | 376 | OE1 | GLU | A1050 | 11.108 | 6.330 | 2.760 | 1.00 | 30.35 | O |
| ATOM | 377 | OE2 | GLU | A1050 | 8.909 | 6.394 | 2.882 | 1.00 | 30.91 | O |
| ATOM | 378 | C | GLU | A1050 | 9.885 | 9.454 | 1.279 | 1.00 | 22.26 | C |
| ATOM | 379 | O | GLU | A1050 | 10.627 | 9.288 | 2.253 | 1.00 | 23.12 | O |
| ATOM | 380 | N | TYR | A1051 | 10.209 | 10.245 | 0.262 | 1.00 | 22.07 | N |
| ATOM | 381 | CA | TYR | A1051 | 11.478 | 10.948 | 0.262 | 1.00 | 21.63 | C |
| ATOM | 382 | CB | TYR | A1051 | 11.686 | 11.704 | −1.054 | 1.00 | 20.34 | C |
| ATOM | 383 | CG | TYR | A1051 | 12.992 | 12.472 | −1.082 | 1.00 | 20.50 | C |
| ATOM | 384 | CD1 | TYR | A1051 | 13.055 | 13.801 | −0.659 | 1.00 | 20.43 | C |
| ATOM | 385 | CE1 | TYR | A1051 | 14.269 | 14.490 | −0.621 | 1.00 | 20.01 | C |
| ATOM | 386 | CD2 | TYR | A1051 | 14.179 | 11.849 | −1.475 | 1.00 | 20.86 | C |
| ATOM | 387 | CE2 | TYR | A1051 | 15.400 | 12.531 | −1.441 | 1.00 | 21.25 | C |
| ATOM | 388 | CZ | TYR | A1051 | 15.432 | 13.852 | −1.010 | 1.00 | 19.89 | C |
| ATOM | 389 | OH | TYR | A1051 | 16.630 | 14.525 | −0.959 | 1.00 | 20.04 | O |
| ATOM | 390 | C | TYR | A1051 | 11.557 | 11.920 | 1.434 | 1.00 | 21.88 | C |
| ATOM | 391 | O | TYR | A1051 | 12.557 | 11.963 | 2.152 | 1.00 | 20.59 | O |
| ATOM | 392 | N | LEU | A1052 | 10.499 | 12.702 | 1.621 | 1.00 | 21.63 | N |
| ATOM | 393 | CA | LEU | A1052 | 10.452 | 13.671 | 2.706 | 1.00 | 21.68 | C |
| ATOM | 394 | CB | LEU | A1052 | 9.179 | 14.513 | 2.590 | 1.00 | 20.47 | C |
| ATOM | 395 | CG | LEU | A1052 | 8.808 | 15.418 | 3.765 | 1.00 | 22.03 | C |
| ATOM | 396 | CD1 | LEU | A1052 | 9.962 | 16.348 | 4.120 | 1.00 | 21.80 | C |
| ATOM | 397 | CD2 | LEU | A1052 | 7.566 | 16.209 | 3.381 | 1.00 | 20.67 | C |
| ATOM | 398 | C | LEU | A1052 | 10.504 | 12.964 | 4.055 | 1.00 | 20.98 | C |
| ATOM | 399 | O | LEU | A1052 | 11.314 | 13.306 | 4.919 | 1.00 | 21.63 | O |
| ATOM | 400 | N | ALA | A1053 | 9.639 | 11.971 | 4.223 | 1.00 | 19.65 | N |
| ATOM | 401 | CA | ALA | A1053 | 9.578 | 11.190 | 5.457 | 1.00 | 18.20 | C |
| ATOM | 402 | CB | ALA | A1053 | 8.567 | 10.054 | 5.299 | 1.00 | 18.13 | C |
| ATOM | 403 | C | ALA | A1053 | 10.952 | 10.614 | 5.818 | 1.00 | 17.32 | C |
| ATOM | 404 | O | ALA | A1053 | 11.356 | 10.642 | 6.982 | 1.00 | 15.65 | O |
| ATOM | 405 | N | GLY | A1054 | 11.660 | 10.092 | 4.816 | 1.00 | 16.84 | N |
| ATOM | 406 | CA | GLY | A1054 | 12.978 | 9.516 | 5.049 | 1.00 | 16.81 | C |
| ATOM | 407 | C | GLY | A1054 | 13.981 | 10.540 | 5.542 | 1.00 | 16.80 | C |
| ATOM | 408 | O | GLY | A1054 | 14.788 | 10.270 | 6.439 | 1.00 | 16.69 | O |
| ATOM | 409 | N | ARG | A1055 | 13.946 | 11.728 | 4.952 | 1.00 | 18.02 | N |
| ATOM | 410 | CA | ARG | A1055 | 14.866 | 12.772 | 5.364 | 1.00 | 18.20 | C |
| ATOM | 411 | CB | ARG | A1055 | 14.811 | 13.928 | 4.371 | 1.00 | 19.98 | C |
| ATOM | 412 | CG | ARG | A1055 | 15.908 | 13.873 | 3.303 | 1.00 | 19.89 | C |
| ATOM | 413 | CD | ARG | A1055 | 15.963 | 12.534 | 2.565 | 1.00 | 21.10 | C |
| ATOM | 414 | NE | ARG | A1055 | 17.109 | 12.487 | 1.652 | 1.00 | 21.61 | N |
| ATOM | 415 | CZ | ARG | A1055 | 17.449 | 11.426 | 0.925 | 1.00 | 21.69 | C |
| ATOM | 416 | NH1 | ARG | A1055 | 16.730 | 10.311 | 0.999 | 1.00 | 20.97 | N |
| ATOM | 417 | NH2 | ARG | A1055 | 18.508 | 11.481 | 0.122 | 1.00 | 17.88 | N |
| ATOM | 418 | C | ARG | A1055 | 14.534 | 13.243 | 6.781 | 1.00 | 18.74 | C |
| ATOM | 419 | O | ARG | A1055 | 15.433 | 13.506 | 7.585 | 1.00 | 16.13 | O |
| ATOM | 420 | N | TRP | A1056 | 13.243 | 13.324 | 7.086 | 1.00 | 17.18 | N |
| ATOM | 421 | CA | TRP | A1056 | 12.793 | 13.732 | 8.412 | 1.00 | 19.43 | C |
| ATOM | 422 | CB | TRP | A1056 | 11.267 | 13.703 | 8.468 | 1.00 | 21.99 | C |
| ATOM | 423 | CG | TRP | A1056 | 10.694 | 14.264 | 9.713 | 1.00 | 26.53 | C |
| ATOM | 424 | CD2 | TRP | A1056 | 10.311 | 13.533 | 10.877 | 1.00 | 28.20 | C |
| ATOM | 425 | CE2 | TRP | A1056 | 9.816 | 14.468 | 11.813 | 1.00 | 28.53 | C |
| ATOM | 426 | CE3 | TRP | A1056 | 10.333 | 12.177 | 11.222 | 1.00 | 29.04 | C |
| ATOM | 427 | CD1 | TRP | A1056 | 10.431 | 15.581 | 9.978 | 1.00 | 26.38 | C |
| ATOM | 428 | NE1 | TRP | A1056 | 9.905 | 15.708 | 11.237 | 1.00 | 29.97 | N |
| ATOM | 429 | CZ2 | TRP | A1056 | 9.347 | 14.090 | 13.073 | 1.00 | 30.15 | C |
| ATOM | 430 | CZ3 | TRP | A1056 | 9.868 | 11.801 | 12.476 | 1.00 | 31.30 | C |
| ATOM | 431 | CH2 | TRP | A1056 | 9.379 | 12.755 | 13.386 | 1.00 | 31.45 | C |
| ATOM | 432 | C | TRP | A1056 | 13.343 | 12.710 | 9.412 | 1.00 | 18.72 | C |
| ATOM | 433 | O | TRP | A1056 | 13.992 | 13.063 | 10.397 | 1.00 | 16.51 | O |
| ATOM | 434 | N | SER | A1057 | 13.084 | 11.435 | 9.137 | 1.00 | 17.50 | N |
| ATOM | 435 | CA | SER | A1057 | 13.532 | 10.352 | 10.011 | 1.00 | 19.23 | C |
| ATOM | 436 | CB | SER | A1057 | 13.053 | 9.009 | 9.448 | 1.00 | 21.41 | C |
| ATOM | 437 | OG | SER | A1057 | 13.243 | 7.971 | 10.388 | 1.00 | 25.06 | O |
| ATOM | 438 | C | SER | A1057 | 15.053 | 10.320 | 10.201 | 1.00 | 19.01 | C |
| ATOM | 439 | O | SER | A1057 | 15.549 | 10.120 | 11.317 | 1.00 | 19.71 | O |
| ATOM | 440 | N | ALA | A1058 | 15.789 | 10.511 | 9.111 | 1.00 | 16.54 | N |
| ATOM | 441 | CA | ALA | A1058 | 17.243 | 10.495 | 9.162 | 1.00 | 16.02 | C |
| ATOM | 442 | CB | ALA | A1058 | 17.798 | 10.607 | 7.757 | 1.00 | 15.16 | C |
| ATOM | 443 | C | ALA | A1058 | 17.790 | 11.616 | 10.035 | 1.00 | 15.70 | C |
| ATOM | 444 | O | ALA | A1058 | 18.707 | 11.411 | 10.836 | 1.00 | 16.78 | O |
| ATOM | 445 | N | LYS | A1059 | 17.221 | 12.805 | 9.884 | 1.00 | 16.69 | N |
| ATOM | 446 | CA | LYS | A1059 | 17.664 | 13.957 | 10.648 | 1.00 | 18.17 | C |
| ATOM | 447 | CB | LYS | A1059 | 17.079 | 15.240 | 10.042 | 1.00 | 18.92 | C |
| ATOM | 448 | CG | LYS | A1059 | 17.536 | 15.450 | 8.588 | 1.00 | 19.75 | C |
| ATOM | 449 | CD | LYS | A1059 | 17.059 | 16.773 | 8.003 | 1.00 | 19.99 | C |
| ATOM | 450 | CE | LYS | A1059 | 17.531 | 16.933 | 6.549 | 1.00 | 19.86 | C |
| ATOM | 451 | NZ | LYS | A1059 | 17.304 | 18.322 | 6.031 | 1.00 | 20.25 | N |
| ATOM | 452 | C | LYS | A1059 | 17.302 | 13.818 | 12.116 | 1.00 | 19.11 | C |
| ATOM | 453 | O | LYS | A1059 | 18.067 | 14.222 | 12.995 | 1.00 | 18.56 | O |
| ATOM | 454 | N | GLU | A1060 | 16.143 | 13.225 | 12.374 | 1.00 | 18.52 | N |
| ATOM | 455 | CA | GLU | A1060 | 15.679 | 12.998 | 13.736 | 1.00 | 21.84 | C |
| ATOM | 456 | CB | GLU | A1060 | 14.247 | 12.448 | 13.704 | 1.00 | 26.26 | C |
| ATOM | 457 | CG | GLU | A1060 | 13.428 | 12.722 | 14.952 | 1.00 | 33.87 | C |
| ATOM | 458 | CD | GLU | A1060 | 13.083 | 14.192 | 15.130 | 1.00 | 35.47 | C |
| ATOM | 459 | OE1 | GLU | A1060 | 12.298 | 14.733 | 14.322 | 1.00 | 38.74 | O |
| ATOM | 460 | OE2 | GLU | A1060 | 13.601 | 14.811 | 16.082 | 1.00 | 39.14 | O |
| ATOM | 461 | O | GLU | A1060 | 16.631 | 11.982 | 14.390 | 1.00 | 21.03 | C |
| ATOM | 462 | O | GLU | A1060 | 17.045 | 12.149 | 15.544 | 1.00 | 18.91 | O |
| ATOM | 463 | N | ALA | A1061 | 16.991 | 10.943 | 13.635 | 1.00 | 18.55 | N |
| ATOM | 464 | CA | ALA | A1061 | 17.898 | 9.910 | 14.131 | 1.00 | 18.39 | C |
| ATOM | 465 | CB | ALA | A1061 | 18.017 | 8.777 | 13.113 | 1.00 | 15.08 | C |
| ATOM | 466 | C | ALA | A1061 | 19.283 | 10.490 | 14.429 | 1.00 | 18.00 | C |
| ATOM | 467 | O | ALA | A1061 | 19.905 | 10.151 | 15.436 | 1.00 | 17.16 | O |
| ATOM | 468 | N | PHE | A1062 | 19.768 | 11.352 | 13.542 | 1.00 | 18.20 | N |
| ATOM | 469 | CA | PHE | A1062 | 21.069 | 11.970 | 13.741 | 1.00 | 20.25 | C |
| ATOM | 470 | CB | PHE | A1062 | 21.433 | 12.855 | 12.546 | 1.00 | 20.89 | C |
| ATOM | 471 | CG | PHE | A1062 | 22.721 | 13.605 | 12.730 | 1.00 | 23.32 | C |
| ATOM | 472 | CD1 | PHE | A1062 | 22.761 | 14.776 | 13.489 | 1.00 | 24.72 | C |
| ATOM | 473 | CD2 | PHE | A1062 | 23.908 | 13.112 | 12.194 | 1.00 | 23.55 | C |
| ATOM | 474 | CE1 | PHE | A1062 | 23.973 | 15.443 | 13.717 | 1.00 | 24.71 | C |
| ATOM | 475 | CE2 | PHE | A1062 | 25.119 | 13.766 | 12.414 | 1.00 | 23.92 | C |
| ATOM | 476 | CZ | PHE | A1062 | 25.150 | 14.934 | 13.179 | 1.00 | 25.42 | C |
| ATOM | 477 | C | PHE | A1062 | 21.090 | 12.800 | 15.026 | 1.00 | 21.33 | C |
| ATOM | 478 | O | PHE | A1062 | 22.035 | 12.726 | 15.809 | 1.00 | 20.18 | O |
| ATOM | 479 | N | SER | A1063 | 20.045 | 13.593 | 15.244 | 1.00 | 22.52 | N |
| ATOM | 480 | CA | SER | A1063 | 19.973 | 14.420 | 16.445 | 1.00 | 24.40 | C |
| ATOM | 481 | CB | SER | A1063 | 18.679 | 15.230 | 16.447 | 1.00 | 25.96 | C |
| ATOM | 482 | OG | SER | A1063 | 18.670 | 16.112 | 15.338 | 1.00 | 30.50 | O |
| ATOM | 483 | C | SER | A1063 | 20.060 | 13.562 | 17.704 | 1.00 | 24.30 | C |
| ATOM | 484 | O | SER | A1063 | 20.714 | 13.938 | 18.682 | 1.00 | 23.77 | O |
| ATOM | 485 | N | LYS | A1064 | 19.407 | 12.406 | 17.678 | 1.00 | 22.33 | N |
| ATOM | 486 | CA | LYS | A1064 | 19.451 | 11.513 | 18.823 | 1.00 | 23.04 | C |
| ATOM | 487 | CB | LYS | A1064 | 18.405 | 10.406 | 18.684 | 1.00 | 23.38 | C |
| ATOM | 488 | CG | LYS | A1064 | 16.971 | 10.881 | 18.903 | 1.00 | 25.10 | C |
| ATOM | 489 | CD | LYS | A1064 | 15.986 | 9.734 | 18.726 | 1.00 | 28.41 | C |
| ATOM | 490 | CE | LYS | A1064 | 14.548 | 10.194 | 18.924 | 1.00 | 32.98 | C |
| ATOM | 491 | NZ | LYS | A1064 | 13.566 | 9.195 | 18.410 | 1.00 | 35.52 | N |
| ATOM | 492 | C | LYS | A1064 | 20.845 | 10.907 | 18.949 | 1.00 | 22.52 | C |
| ATOM | 493 | O | LYS | A1064 | 21.285 | 10.572 | 20.047 | 1.00 | 21.41 | O |
| ATOM | 494 | N | ALA | A1065 | 21.539 | 10.774 | 17.820 | 1.00 | 21.47 | N |
| ATOM | 495 | CA | ALA | A1065 | 22.885 | 10.220 | 17.826 | 1.00 | 22.53 | C |
| ATOM | 496 | CB | ALA | A1065 | 23.379 | 9.980 | 16.399 | 1.00 | 20.33 | C |
| ATOM | 497 | C | ALA | A1065 | 23.796 | 11.207 | 18.537 | 1.00 | 24.41 | C |
| ATOM | 498 | O | ALA | A1065 | 24.751 | 10.811 | 19.201 | 1.00 | 24.00 | O |
| ATOM | 499 | N | MET | A1066 | 23.499 | 12.496 | 18.390 | 1.00 | 28.05 | N |
| ATOM | 500 | CA | MET | A1066 | 24.283 | 13.530 | 19.049 | 1.00 | 32.04 | C |
| ATOM | 501 | CB | MET | A1066 | 24.167 | 14.859 | 18.309 | 1.00 | 34.23 | C |
| ATOM | 502 | CG | MET | A1066 | 24.913 | 14.895 | 16.997 | 1.00 | 40.28 | C |
| ATOM | 503 | SD | MET | A1066 | 25.423 | 16.574 | 16.602 | 1.00 | 48.25 | S |
| ATOM | 504 | CE | MET | A1066 | 27.174 | 16.492 | 17.024 | 1.00 | 46.99 | C |
| ATOM | 505 | C | MET | A1066 | 23.826 | 13.716 | 20.493 | 1.00 | 33.24 | C |
| ATOM | 506 | O | MET | A1066 | 24.456 | 14.443 | 21.254 | 1.00 | 33.60 | O |
| ATOM | 507 | N | GLY | A1067 | 22.720 | 13.069 | 20.856 | 1.00 | 33.56 | N |
| ATOM | 508 | CA | GLY | A1067 | 22.217 | 13.159 | 22.216 | 1.00 | 34.99 | C |
| ATOM | 509 | C | GLY | A1067 | 21.311 | 14.339 | 22.519 | 1.00 | 36.72 | C |
| ATOM | 510 | O | GLY | A1067 | 21.237 | 14.787 | 23.666 | 1.00 | 34.92 | O |
| ATOM | 511 | N | THR | A1068 | 20.616 | 14.845 | 21.504 | 1.00 | 38.07 | N |
| ATOM | 512 | CA | THR | A1068 | 19.716 | 15.975 | 21.699 | 1.00 | 39.81 | C |
| ATOM | 513 | CB | THR | A1068 | 20.455 | 17.316 | 21.504 | 1.00 | 40.69 | C |
| ATOM | 514 | OG1 | THR | A1068 | 19.535 | 18.400 | 21.687 | 1.00 | 42.07 | O |
| ATOM | 515 | CG2 | THR | A1068 | 21.062 | 17.396 | 20.114 | 1.00 | 40.32 | C |
| ATOM | 516 | C | THR | A1068 | 18.520 | 15.922 | 20.752 | 1.00 | 40.62 | C |
| ATOM | 517 | O | THR | A1068 | 18.293 | 14.915 | 20.077 | 1.00 | 41.29 | O |
| ATOM | 518 | N | GLY | A1069 | 17.753 | 17.007 | 20.716 | 1.00 | 40.61 | N |
| ATOM | 519 | CA | GLY | A1069 | 16.586 | 17.061 | 19.856 | 1.00 | 40.92 | C |
| ATOM | 520 | C | GLY | A1069 | 16.835 | 17.849 | 18.587 | 1.00 | 40.96 | C |
| ATOM | 521 | O | GLY | A1069 | 17.705 | 18.722 | 18.547 | 1.00 | 41.30 | O |
| ATOM | 522 | N | ILE | A1070 | 16.065 | 17.545 | 17.547 | 1.00 | 40.13 | N |
| ATOM | 523 | CA | ILE | A1070 | 16.207 | 18.224 | 16.266 | 1.00 | 40.90 | C |
| ATOM | 524 | CB | ILE | A1070 | 15.288 | 17.591 | 15.200 | 1.00 | 40.70 | C |
| ATOM | 525 | CG2 | ILE | A1070 | 13.828 | 17.774 | 15.593 | 1.00 | 40.55 | C |
| ATOM | 526 | CG1 | ILE | A1070 | 15.557 | 18.227 | 13.835 | 1.00 | 41.43 | C |
| ATOM | 527 | CD1 | ILE | A1070 | 16.980 | 18.055 | 13.352 | 1.00 | 39.85 | C |
| ATOM | 528 | C | ILE | A1070 | 15.876 | 19.712 | 16.387 | 1.00 | 41.49 | C |
| ATOM | 529 | O | ILE | A1070 | 16.354 | 20.533 | 15.598 | 1.00 | 41.31 | O |
| ATOM | 530 | N | SER | A1071 | 15.058 | 20.054 | 17.379 | 1.00 | 41.78 | N |
| ATOM | 531 | CA | SER | A1071 | 14.672 | 21.442 | 17.594 | 1.00 | 42.83 | C |
| ATOM | 532 | CB | SER | A1071 | 13.546 | 21.531 | 18.633 | 1.00 | 43.42 | C |
| ATOM | 533 | OG | SER | A1071 | 13.938 | 20.980 | 19.877 | 1.00 | 44.55 | O |
| ATOM | 534 | C | SER | A1071 | 15.856 | 22.295 | 18.039 | 1.00 | 43.32 | C |
| ATOM | 535 | O | SER | A1071 | 15.792 | 23.523 | 17.984 | 1.00 | 44.46 | O |
| ATOM | 536 | N | LYS | A1072 | 16.938 | 21.648 | 18.472 | 1.00 | 42.56 | N |
| ATOM | 537 | CA | LYS | A1072 | 18.123 | 22.377 | 18.911 | 1.00 | 41.56 | C |
| ATOM | 538 | CB | LYS | A1072 | 18.744 | 21.706 | 20.141 | 1.00 | 41.25 | C |
| ATOM | 539 | CG | LYS | A1072 | 19.600 | 20.489 | 19.844 | 0.05 | 41.20 | C |
| ATOM | 540 | CD | LYS | A1072 | 21.069 | 20.860 | 19.724 | 0.05 | 41.09 | C |
| ATOM | 541 | CE | LYS | A1072 | 21.602 | 21.414 | 21.037 | 0.05 | 41.02 | C |
| ATOM | 542 | NZ | LYS | A1072 | 21.470 | 20.437 | 22.152 | 0.05 | 40.94 | N |
| ATOM | 543 | C | LYS | A1072 | 19.139 | 22.462 | 17.777 | 1.00 | 41.40 | C |
| ATOM | 544 | O | LYS | A1072 | 19.840 | 23.462 | 17.643 | 1.00 | 41.76 | O |
| ATOM | 545 | N | LEU | A1073 | 19.220 | 21.413 | 16.960 | 1.00 | 40.37 | N |
| ATOM | 546 | CA | LEU | A1073 | 20.142 | 21.411 | 15.829 | 1.00 | 38.42 | C |
| ATOM | 547 | CB | LEU | A1073 | 20.439 | 19.985 | 15.339 | 1.00 | 39.84 | C |
| ATOM | 548 | CG | LEU | A1073 | 21.489 | 19.100 | 16.015 | 1.00 | 40.56 | C |
| ATOM | 549 | CD1 | LEU | A1073 | 22.585 | 19.956 | 16.635 | 1.00 | 41.19 | C |
| ATOM | 550 | CD2 | LEU | A1073 | 20.820 | 18.243 | 17.064 | 1.00 | 42.23 | C |
| ATOM | 551 | C | LEU | A1073 | 19.556 | 22.198 | 14.664 | 1.00 | 36.59 | C |
| ATOM | 552 | O | LEU | A1073 | 20.205 | 23.091 | 14.123 | 1.00 | 36.83 | O |
| ATOM | 553 | N | GLY | A1074 | 18.326 | 21.849 | 14.285 | 1.00 | 34.67 | N |
| ATOM | 554 | CA | GLY | A1074 | 17.653 | 22.500 | 13.172 | 1.00 | 31.12 | C |
| ATOM | 555 | C | GLY | A1074 | 17.746 | 21.631 | 11.927 | 1.00 | 29.81 | C |
| ATOM | 556 | O | GLY | A1074 | 18.830 | 21.176 | 11.576 | 1.00 | 29.20 | O |
| ATOM | 557 | N | PHE | A1075 | 16.619 | 21.396 | 11.260 | 1.00 | 29.29 | N |
| ATOM | 558 | CA | PHE | A1075 | 16.595 | 20.565 | 10.051 | 1.00 | 29.15 | C |
| ATOM | 559 | CB | PHE | A1075 | 15.163 | 20.442 | 9.502 | 1.00 | 28.80 | C |
| ATOM | 560 | CG | PHE | A1075 | 14.244 | 19.610 | 10.353 | 1.00 | 29.16 | C |
| ATOM | 561 | CD1 | PHE | A1075 | 13.306 | 20.212 | 11.183 | 1.00 | 29.51 | C |
| ATOM | 562 | CD2 | PHE | A1075 | 14.309 | 18.221 | 10.315 | 1.00 | 28.39 | C |
| ATOM | 563 | CE1 | PHE | A1075 | 12.440 | 19.443 | 11.964 | 1.00 | 28.84 | C |
| ATOM | 564 | CE2 | PHE | A1075 | 13.451 | 17.445 | 11.089 | 1.00 | 30.94 | C |
| ATOM | 565 | CZ | PHE | A1075 | 12.514 | 18.059 | 11.916 | 1.00 | 29.27 | C |
| ATOM | 566 | C | PHE | A1075 | 17.500 | 21.072 | 8.922 | 1.00 | 29.25 | C |
| ATOM | 567 | O | PHE | A1075 | 18.096 | 20.275 | 8.193 | 1.00 | 27.78 | O |
| ATOM | 568 | N | GLN | A1076 | 17.589 | 22.393 | 8.772 | 1.00 | 29.64 | N |
| ATOM | 569 | CA | GLN | A1076 | 18.397 | 23.008 | 7.717 | 1.00 | 29.73 | C |
| ATOM | 570 | CB | GLN | A1076 | 18.046 | 24.497 | 7.593 | 1.00 | 31.55 | C |
| ATOM | 571 | CG | GLN | A1076 | 16.585 | 24.746 | 7.253 | 1.00 | 31.77 | C |
| ATOM | 572 | CD | GLN | A1076 | 16.202 | 24.181 | 5.895 | 1.00 | 32.15 | C |
| ATOM | 573 | OE1 | GLN | A1076 | 15.044 | 23.849 | 5.652 | 1.00 | 34.09 | O |
| ATOM | 574 | NE2 | GLN | A1076 | 17.174 | 24.084 | 5.002 | 1.00 | 32.35 | N |
| ATOM | 575 | C | GLN | A1076 | 19.888 | 22.845 | 7.975 | 1.00 | 31.01 | C |
| ATOM | 576 | O | GLN | A1076 | 20.724 | 23.105 | 7.099 | 1.00 | 30.59 | O |
| ATOM | 577 | N | ASP | A1077 | 20.211 | 22.401 | 9.183 | 1.00 | 30.30 | N |
| ATOM | 578 | CA | ASP | A1077 | 21.592 | 22.182 | 9.588 | 1.00 | 30.99 | C |
| ATOM | 579 | CB | ASP | A1077 | 21.684 | 22.266 | 11.111 | 1.00 | 34.42 | C |
| ATOM | 580 | CG | ASP | A1077 | 22.894 | 23.034 | 11.578 | 1.00 | 38.94 | C |
| ATOM | 581 | OD1 | ASP | A1077 | 24.025 | 22.549 | 11.358 | 1.00 | 40.63 | O |
| ATOM | 582 | OD2 | ASP | A1077 | 22.710 | 24.127 | 12.162 | 1.00 | 41.18 | O |
| ATOM | 583 | C | ASP | A1077 | 22.066 | 20.803 | 9.114 | 1.00 | 28.98 | C |
| ATOM | 584 | O | ASP | A1077 | 23.247 | 20.470 | 9.198 | 1.00 | 27.03 | O |
| ATOM | 585 | N | LEU | A1078 | 21.134 | 19.998 | 8.622 | 1.00 | 26.47 | N |
| ATOM | 586 | CA | LEU | A1078 | 21.472 | 18.665 | 8.155 | 1.00 | 25.11 | C |
| ATOM | 587 | CB | LEU | A1078 | 20.782 | 17.614 | 9.029 | 1.00 | 25.83 | C |
| ATOM | 588 | CG | LEU | A1078 | 21.066 | 17.667 | 10.529 | 1.00 | 25.61 | C |
| ATOM | 589 | CD1 | LEU | A1078 | 20.075 | 16.787 | 11.291 | 1.00 | 26.16 | C |
| ATOM | 590 | CD2 | LEU | A1078 | 22.494 | 17.227 | 10.775 | 1.00 | 26.52 | C |
| ATOM | 591 | C | LEU | A1078 | 21.015 | 18.495 | 6.723 | 1.00 | 24.62 | C |
| ATOM | 592 | O | LEU | A1078 | 20.052 | 19.125 | 6.296 | 1.00 | 25.13 | O |
| ATOM | 593 | N | GLU | A1079 | 21.710 | 17.648 | 5.975 | 1.00 | 23.38 | N |
| ATOM | 594 | CA | GLU | A1079 | 21.312 | 17.388 | 4.603 | 1.00 | 22.76 | C |
| ATOM | 595 | CB | GLU | A1079 | 22.008 | 18.338 | 3.639 | 1.00 | 24.57 | C |
| ATOM | 596 | CG | GLU | A1079 | 21.425 | 18.299 | 2.241 | 1.00 | 25.55 | C |
| ATOM | 597 | CD | GLU | A1079 | 22.219 | 19.146 | 1.274 | 1.00 | 28.95 | C |
| ATOM | 598 | OE1 | GLU | A1079 | 23.415 | 18.844 | 1.074 | 1.00 | 28.46 | O |
| ATOM | 599 | OE2 | GLU | A1079 | 21.652 | 20.114 | 0.722 | 1.00 | 31.06 | O |
| ATOM | 600 | C | GLU | A1079 | 21.639 | 15.958 | 4.220 | 1.00 | 21.67 | C |
| ATOM | 601 | O | GLU | A1079 | 22.747 | 15.476 | 4.458 | 1.00 | 21.79 | O |
| ATOM | 602 | N | VAL | A1080 | 20.667 | 15.283 | 3.626 | 1.00 | 18.92 | N |
| ATOM | 603 | CA | VAL | A1080 | 20.861 | 13.904 | 3.208 | 1.00 | 19.27 | C |
| ATOM | 604 | CB | VAL | A1080 | 19.836 | 12.967 | 3.881 | 1.00 | 18.84 | C |
| ATOM | 605 | CG1 | VAL | A1080 | 20.181 | 11.504 | 3.578 | 1.00 | 18.64 | C |
| ATOM | 606 | CG2 | VAL | A1080 | 19.801 | 13.230 | 5.368 | 1.00 | 18.66 | C |
| ATOM | 607 | C | VAL | A1080 | 20.690 | 13.798 | 1.696 | 1.00 | 19.85 | C |
| ATOM | 608 | O | VAL | A1080 | 19.596 | 14.003 | 1.169 | 1.00 | 19.06 | O |
| ATOM | 609 | N | LEU | A1081 | 21.780 | 13.497 | 1.003 | 1.00 | 19.76 | N |
| ATOM | 610 | CA | LEU | A1081 | 21.742 | 13.332 | −0.442 | 1.00 | 20.15 | C |
| ATOM | 611 | CB | LEU | A1081 | 22.808 | 14.210 | −1.111 | 1.00 | 19.63 | C |
| ATOM | 612 | CG | LEU | A1081 | 22.640 | 15.717 | −0.878 | 1.00 | 19.82 | C |
| ATOM | 613 | CD1 | LEU | A1081 | 23.817 | 16.488 | −1.475 | 1.00 | 19.43 | C |
| ATOM | 614 | CD2 | LEU | A1081 | 21.326 | 16.171 | −1.485 | 1.00 | 18.80 | C |
| ATOM | 615 | C | LEU | A1081 | 22.037 | 11.861 | −0.696 | 1.00 | 20.78 | C |
| ATOM | 616 | O | LEU | A1081 | 22.097 | 11.067 | 0.242 | 1.00 | 19.52 | O |
| ATOM | 617 | N | ASN | A1082 | 22.224 | 11.495 | −1.957 | 1.00 | 21.64 | N |
| ATOM | 618 | CA | ASN | A1082 | 22.521 | 10.110 | −2.301 | 1.00 | 22.51 | C |
| ATOM | 619 | CB | ASN | A1082 | 21.430 | 9.574 | −3.230 | 1.00 | 21.83 | C |
| ATOM | 620 | CG | ASN | A1082 | 20.134 | 9.281 | −2.485 | 1.00 | 24.97 | C |
| ATOM | 621 | OD1 | ASN | A1082 | 19.930 | 8.174 | −1.973 | 1.00 | 26.72 | O |
| ATOM | 622 | ND2 | ASN | A1082 | 19.264 | 10.275 | −2.402 | 1.00 | 23.64 | N |
| ATOM | 623 | C | ASN | A1082 | 23.897 | 10.029 | −2.954 | 1.00 | 22.59 | C |
| ATOM | 624 | O | ASN | A1082 | 24.196 | 10.786 | −3.876 | 1.00 | 23.10 | O |
| ATOM | 625 | N | ASN | A1083 | 24.738 | 9.118 | −2.472 | 1.00 | 21.81 | N |
| ATOM | 626 | CA | ASN | A1083 | 26.079 | 8.998 | −3.021 | 1.00 | 24.05 | C |
| ATOM | 627 | CB | ASN | A1083 | 27.011 | 8.298 | −2.018 | 1.00 | 21.32 | C |
| ATOM | 628 | CG | ASN | A1083 | 26.634 | 6.849 | −1.748 | 1.00 | 23.86 | C |
| ATOM | 629 | OD1 | ASN | A1083 | 27.083 | 6.269 | −0.752 | 1.00 | 23.67 | O |
| ATOM | 630 | ND2 | ASN | A1083 | 25.835 | 6.249 | −2.631 | 1.00 | 20.56 | N |
| ATOM | 631 | C | ASN | A1083 | 26.112 | 8.315 | −4.385 | 1.00 | 26.50 | C |
| ATOM | 632 | O | ASN | A1083 | 25.065 | 7.986 | −4.951 | 1.00 | 25.00 | O |
| ATOM | 633 | N | GLU | A1084 | 27.313 | 8.118 | −4.921 | 1.00 | 28.34 | N |
| ATOM | 634 | CA | GLU | A1084 | 27.447 | 7.514 | −6.237 | 1.00 | 32.10 | C |
| ATOM | 635 | CB | GLU | A1084 | 28.907 | 7.561 | −6.700 | 1.00 | 32.77 | C |
| ATOM | 636 | CG | GLU | A1084 | 29.885 | 6.749 | −5.877 | 1.00 | 35.30 | C |
| ATOM | 637 | CD | GLU | A1084 | 31.327 | 7.041 | −6.263 | 1.00 | 38.54 | C |
| ATOM | 638 | OE1 | GLU | A1084 | 31.628 | 7.058 | −7.478 | 1.00 | 39.35 | O |
| ATOM | 639 | OE2 | GLU | A1084 | 32.160 | 7.252 | −5.356 | 1.00 | 40.56 | O |
| ATOM | 640 | C | GLU | A1084 | 26.906 | 6.092 | −6.307 | 1.00 | 32.37 | C |
| ATOM | 641 | O | GLU | A1084 | 26.582 | 5.610 | −7.386 | 1.00 | 34.17 | O |
| ATOM | 642 | N | ARG | A1085 | 26.811 | 5.426 | −5.158 | 1.00 | 31.86 | N |
| ATOM | 643 | CA | ARG | A1085 | 26.278 | 4.069 | −5.111 | 1.00 | 29.86 | C |
| ATOM | 644 | CB | ARG | A1085 | 26.967 | 3.259 | −4.009 | 1.00 | 30.40 | C |
| ATOM | 645 | CG | ARG | A1085 | 28.442 | 2.992 | −4.269 | 0.05 | 30.17 | C |
| ATOM | 646 | CD | ARG | A1085 | 28.668 | 2.173 | −5.540 | 0.05 | 30.07 | C |
| ATOM | 647 | NE | ARG | A1085 | 28.539 | 0.730 | −5.333 | 0.05 | 30.12 | N |
| ATOM | 648 | CZ | ARG | A1085 | 27.396 | 0.089 | −5.107 | 0.05 | 30.07 | C |
| ATOM | 649 | NH1 | ARG | A1085 | 26.249 | 0.751 | −5.054 | 0.05 | 30.16 | N |
| ATOM | 650 | NH2 | ARG | A1085 | 27.401 | −1.226 | −4.938 | 0.05 | 30.04 | N |
| ATOM | 651 | C | ARG | A1085 | 24.768 | 4.117 | −4.860 | 1.00 | 28.84 | C |
| ATOM | 652 | O | ARG | A1085 | 24.121 | 3.084 | −4.683 | 1.00 | 28.73 | O |
| ATOM | 653 | N | GLY | A1086 | 24.222 | 5.329 | −4.842 | 1.00 | 26.09 | N |
| ATOM | 654 | CA | GLY | A1086 | 22.797 | 5.514 | −4.635 | 1.00 | 24.49 | C |
| ATOM | 655 | C | GLY | A1086 | 22.286 | 5.493 | −3.202 | 1.00 | 23.98 | C |
| ATOM | 656 | O | GLY | A1086 | 21.090 | 5.649 | −2.975 | 1.00 | 25.43 | O |
| ATOM | 657 | N | ALA | A1087 | 23.172 | 5.306 | −2.233 | 1.00 | 21.90 | N |
| ATOM | 658 | CA | ALA | A1087 | 22.755 | 5.259 | −0.836 | 1.00 | 21.27 | C |
| ATOM | 659 | CB | ALA | A1087 | 23.704 | 4.379 | −0.042 | 1.00 | 21.23 | C |
| ATOM | 660 | C | ALA | A1087 | 22.689 | 6.653 | −0.205 | 1.00 | 21.63 | C |
| ATOM | 661 | O | ALA | A1087 | 23.508 | 7.529 | −0.499 | 1.00 | 19.21 | O |
| ATOM | 662 | N | PRO | A1088 | 21.700 | 6.872 | 0.668 | 1.00 | 20.00 | N |
| ATOM | 663 | CD | PRO | A1088 | 20.666 | 5.919 | 1.105 | 1.00 | 20.94 | C |
| ATOM | 664 | CA | PRO | A1088 | 21.544 | 8.164 | 1.335 | 1.00 | 20.18 | C |
| ATOM | 665 | CB | PRO | A1088 | 20.212 | 8.013 | 2.064 | 1.00 | 20.85 | C |
| ATOM | 666 | CG | PRO | A1088 | 20.183 | 6.545 | 2.390 | 1.00 | 22.84 | C |
| ATOM | 667 | C | PRO | A1088 | 22.714 | 8.380 | 2.281 | 1.00 | 19.50 | C |
| ATOM | 668 | O | PRO | A1088 | 23.220 | 7.431 | 2.887 | 1.00 | 17.61 | O |
| ATOM | 669 | N | TYR | A1089 | 23.164 | 9.621 | 2.395 | 1.00 | 18.16 | N |
| ATOM | 670 | CA | TYR | A1089 | 24.281 | 9.913 | 3.279 | 1.00 | 19.12 | C |
| ATOM | 671 | CB | TYR | A1089 | 25.611 | 9.642 | 2.557 | 1.00 | 20.66 | C |
| ATOM | 672 | CG | TYR | A1089 | 25.977 | 10.698 | 1.534 | 1.00 | 23.33 | C |
| ATOM | 673 | CD1 | TYR | A1089 | 26.869 | 11.724 | 1.853 | 1.00 | 22.08 | C |
| ATOM | 674 | CE1 | TYR | A1089 | 27.211 | 12.699 | 0.916 | 1.00 | 22.82 | C |
| ATOM | 675 | CD2 | TYR | A1089 | 25.426 | 10.673 | 0.249 | 1.00 | 22.30 | C |
| ATOM | 676 | CE2 | TYR | A1089 | 25.752 | 11.642 | −0.692 | 1.00 | 23.44 | C |
| ATOM | 677 | CZ | TYR | A1089 | 26.649 | 12.655 | −0.354 | 1.00 | 25.15 | C |
| ATOM | 678 | OH | TYR | A1089 | 26.987 | 13.614 | −1.288 | 1.00 | 24.63 | O |
| ATOM | 679 | C | TYR | A1089 | 24.205 | 11.363 | 3.699 | 1.00 | 17.64 | C |
| ATOM | 680 | O | TYR | A1089 | 23.578 | 12.178 | 3.023 | 1.00 | 16.44 | O |
| ATOM | 681 | N | PHE | A1090 | 24.824 | 11.691 | 4.825 | 1.00 | 17.81 | N |
| ATOM | 682 | CA | PHE | A1090 | 24.801 | 13.070 | 5.277 | 1.00 | 17.81 | C |
| ATOM | 683 | CB | PHE | A1090 | 25.019 | 13.159 | 6.791 | 1.00 | 17.48 | C |
| ATOM | 684 | CG | PHE | A1090 | 23.777 | 12.873 | 7.595 | 1.00 | 17.01 | C |
| ATOM | 685 | CD1 | PHE | A1090 | 23.434 | 11.568 | 7.938 | 1.00 | 17.02 | C |
| ATOM | 686 | CD2 | PHE | A1090 | 22.919 | 13.910 | 7.962 | 1.00 | 16.23 | C |
| ATOM | 687 | CE1 | PHE | A1090 | 22.247 | 11.293 | 8.632 | 1.00 | 17.95 | C |
| ATOM | 688 | CE2 | PHE | A1090 | 21.732 | 13.647 | 8.656 | 1.00 | 17.65 | C |
| ATOM | 689 | CZ | PHE | A1090 | 21.397 | 12.333 | 8.989 | 1.00 | 16.73 | C |
| ATOM | 690 | C | PHE | A1090 | 25.852 | 13.892 | 4.547 | 1.00 | 20.29 | C |
| ATOM | 691 | O | PHE | A1090 | 27.054 | 13.713 | 4.749 | 1.00 | 19.58 | O |
| ATOM | 692 | N | SER | A1091 | 25.384 | 14.767 | 3.665 | 1.00 | 21.20 | N |
| ATOM | 693 | CA | SER | A1091 | 26.266 | 15.646 | 2.921 | 1.00 | 23.08 | C |
| ATOM | 694 | CB | SER | A1091 | 25.592 | 16.086 | 1.618 | 1.00 | 24.12 | C |
| ATOM | 695 | OG | SER | A1091 | 24.281 | 16.567 | 1.852 | 1.00 | 24.36 | O |
| ATOM | 696 | C | SER | A1091 | 26.580 | 16.856 | 3.806 | 1.00 | 24.80 | C |
| ATOM | 697 | O | SER | A1091 | 27.613 | 17.507 | 3.642 | 1.00 | 25.24 | O |
| ATOM | 698 | N | GLN | A1092 | 25.685 | 17.141 | 4.752 | 1.00 | 24.80 | N |
| ATOM | 699 | CA | GLN | A1092 | 25.853 | 18.254 | 5.688 | 1.00 | 24.09 | C |
| ATOM | 700 | CB | GLN | A1092 | 24.970 | 19.448 | 5.296 | 1.00 | 26.54 | C |
| ATOM | 701 | CG | GLN | A1092 | 25.294 | 20.132 | 3.971 | 1.00 | 30.86 | C |
| ATOM | 702 | CD | GLN | A1092 | 26.711 | 20.678 | 3.907 | 1.00 | 34.83 | C |
| ATOM | 703 | OE1 | GLN | A1092 | 27.257 | 21.147 | 4.910 | 1.00 | 37.27 | O |
| ATOM | 704 | NE2 | GLN | A1092 | 27.308 | 20.637 | 2.717 | 1.00 | 34.99 | N |
| ATOM | 705 | C | GLN | A1092 | 25.453 | 17.803 | 7.096 | 1.00 | 23.97 | C |
| ATOM | 706 | O | GLN | A1092 | 24.343 | 17.295 | 7.300 | 1.00 | 22.36 | O |
| ATOM | 707 | N | ALA | A1093 | 26.356 | 17.989 | 8.057 | 1.00 | 20.55 | N |
| ATOM | 708 | CA | ALA | A1093 | 26.097 | 17.621 | 9.447 | 1.00 | 21.00 | C |
| ATOM | 709 | CB | ALA | A1093 | 25.948 | 16.101 | 9.580 | 1.00 | 17.57 | C |
| ATOM | 710 | C | ALA | A1093 | 27.235 | 18.114 | 10.336 | 1.00 | 22.11 | C |
| ATOM | 711 | O | ALA | A1093 | 28.392 | 18.120 | 9.920 | 1.00 | 23.23 | O |
| ATOM | 712 | N | PRO | A1094 | 26.918 | 18.536 | 11.573 | 1.00 | 23.31 | N |
| ATOM | 713 | CD | PRO | A1094 | 25.550 | 18.680 | 12.111 | 1.00 | 23.07 | C |
| ATOM | 714 | CA | PRO | A1094 | 27.907 | 19.034 | 12.533 | 1.00 | 24.58 | C |
| ATOM | 715 | CB | PRO | A1094 | 27.053 | 19.820 | 13.521 | 1.00 | 24.45 | C |
| ATOM | 716 | CG | PRO | A1094 | 25.798 | 18.997 | 13.575 | 1.00 | 24.77 | C |
| ATOM | 717 | C | PRO | A1094 | 28.687 | 17.909 | 13.219 | 1.00 | 26.12 | C |
| ATOM | 718 | O | PRO | A1094 | 28.675 | 17.791 | 14.445 | 1.00 | 27.56 | O |
| ATOM | 719 | N | PHE | A1095 | 29.353 | 17.079 | 12.423 | 1.00 | 26.05 | N |
| ATOM | 720 | CA | PHE | A1095 | 30.139 | 15.975 | 12.957 | 1.00 | 26.02 | C |
| ATOM | 721 | CB | PHE | A1095 | 29.279 | 14.710 | 13.090 | 1.00 | 27.36 | C |
| ATOM | 722 | CG | PHE | A1095 | 30.016 | 13.544 | 13.674 | 1.00 | 26.42 | C |
| ATOM | 723 | CD1 | PHE | A1095 | 30.208 | 12.385 | 12.931 | 1.00 | 26.59 | C |
| ATOM | 724 | CD2 | PHE | A1095 | 30.571 | 13.625 | 14.950 | 1.00 | 27.10 | C |
| ATOM | 725 | CE1 | PHE | A1095 | 30.946 | 11.324 | 13.446 | 1.00 | 26.29 | C |
| ATOM | 726 | CE2 | PHE | A1095 | 31.312 | 12.574 | 15.475 | 1.00 | 25.68 | C |
| ATOM | 727 | CZ | PHE | A1095 | 31.502 | 11.417 | 14.720 | 1.00 | 27.52 | C |
| ATOM | 728 | C | PHE | A1095 | 31.305 | 15.727 | 12.012 | 1.00 | 26.40 | C |
| ATOM | 729 | O | PHE | A1095 | 31.122 | 15.681 | 10.792 | 1.00 | 25.80 | O |
| ATOM | 730 | N | SER | A1096 | 32.498 | 15.560 | 12.577 | 1.00 | 26.14 | N |
| ATOM | 731 | CA | SER | A1096 | 33.705 | 15.357 | 11.781 | 1.00 | 29.05 | C |
| ATOM | 732 | CB | SER | A1096 | 34.909 | 15.969 | 12.500 | 1.00 | 30.71 | C |
| ATOM | 733 | OG | SER | A1096 | 34.768 | 17.373 | 12.603 | 1.00 | 37.13 | O |
| ATOM | 734 | C | SER | A1096 | 34.039 | 13.919 | 11.413 | 1.00 | 28.85 | C |
| ATOM | 735 | O | SER | A1096 | 34.702 | 13.678 | 10.407 | 1.00 | 29.54 | O |
| ATOM | 736 | N | GLY | A1097 | 33.595 | 12.968 | 12.229 | 1.00 | 28.58 | N |
| ATOM | 737 | CA | GLY | A1097 | 33.888 | 11.573 | 11.957 | 1.00 | 27.16 | C |
| ATOM | 738 | C | GLY | A1097 | 32.991 | 10.948 | 10.907 | 1.00 | 26.54 | C |
| ATOM | 739 | O | GLY | A1097 | 32.406 | 11.648 | 10.077 | 1.00 | 27.09 | O |
| ATOM | 740 | N | LYS | A1098 | 32.888 | 9.624 | 10.931 | 1.00 | 24.94 | N |
| ATOM | 741 | CA | LYS | A1098 | 32.045 | 8.930 | 9.973 | 1.00 | 25.69 | C |
| ATOM | 742 | CB | LYS | A1098 | 32.587 | 7.535 | 9.644 | 1.00 | 29.36 | C |
| ATOM | 743 | CG | LYS | A1098 | 33.687 | 7.015 | 10.547 | 1.00 | 33.98 | C |
| ATOM | 744 | CD | LYS | A1098 | 35.051 | 7.532 | 10.120 | 1.00 | 37.43 | C |
| ATOM | 745 | CE | LYS | A1098 | 36.164 | 6.583 | 10.568 | 1.00 | 39.11 | C |
| ATOM | 746 | NZ | LYS | A1098 | 36.119 | 6.320 | 12.033 | 1.00 | 41.00 | N |
| ATOM | 747 | C | LYS | A1098 | 30.625 | 8.796 | 10.492 | 1.00 | 24.62 | C |
| ATOM | 748 | O | LYS | A1098 | 30.393 | 8.494 | 11.668 | 1.00 | 22.63 | O |
| ATOM | 749 | N | ILE | A1099 | 29.677 | 9.033 | 9.600 | 1.00 | 22.17 | N |
| ATOM | 750 | CA | ILE | A1099 | 28.273 | 8.932 | 9.941 | 1.00 | 21.57 | C |
| ATOM | 751 | CB | ILE | A1099 | 27.514 | 10.193 | 9.523 | 1.00 | 19.45 | C |
| ATOM | 752 | CG2 | ILE | A1099 | 26.039 | 10.056 | 9.905 | 1.00 | 18.01 | C |
| ATOM | 753 | CG1 | ILE | A1099 | 28.155 | 11.419 | 10.177 | 1.00 | 20.05 | C |
| ATOM | 754 | CD1 | ILE | A1099 | 27.677 | 12.738 | 9.613 | 1.00 | 19.45 | C |
| ATOM | 755 | C | ILE | A1099 | 27.695 | 7.748 | 9.184 | 1.00 | 21.84 | C |
| ATOM | 756 | O | ILE | A1099 | 27.597 | 7.775 | 7.958 | 1.00 | 24.63 | O |
| ATOM | 757 | N | TRP | A1100 | 27.334 | 6.699 | 9.907 | 1.00 | 20.85 | N |
| ATOM | 758 | CA | TRP | A1100 | 26.760 | 5.532 | 9.270 | 1.00 | 19.86 | C |
| ATOM | 759 | CB | TRP | A1100 | 27.172 | 4.260 | 10.009 | 1.00 | 18.86 | C |
| ATOM | 760 | CG | TRP | A1100 | 28.652 | 4.007 | 9.945 | 1.00 | 19.33 | C |
| ATOM | 761 | CD2 | TRP | A1100 | 29.370 | 3.395 | 8.864 | 1.00 | 20.59 | C |
| ATOM | 762 | CE2 | TRP | A1100 | 30.743 | 3.416 | 9.209 | 1.00 | 20.05 | C |
| ATOM | 763 | CE3 | TRP | A1100 | 28.987 | 2.835 | 7.634 | 1.00 | 19.76 | C |
| ATOM | 764 | CD1 | TRP | A1100 | 29.590 | 4.362 | 10.873 | 1.00 | 19.35 | C |
| ATOM | 765 | NE1 | TRP | A1100 | 30.846 | 4.010 | 10.439 | 1.00 | 18.87 | N |
| ATOM | 766 | CZ2 | TRP | A1100 | 31.739 | 2.895 | 8.369 | 1.00 | 19.48 | C |
| ATOM | 767 | CZ3 | TRP | A1100 | 29.978 | 2.317 | 6.796 | 1.00 | 22.42 | C |
| ATOM | 768 | CH2 | TRP | A1100 | 31.343 | 2.354 | 7.172 | 1.00 | 20.56 | C |
| ATOM | 769 | C | TRP | A1100 | 25.251 | 5.718 | 9.294 | 1.00 | 20.31 | C |
| ATOM | 770 | O | TRP | A1100 | 24.633 | 5.795 | 10.357 | 1.00 | 20.18 | O |
| ATOM | 771 | N | LEU | A1101 | 24.663 | 5.829 | 8.113 | 1.00 | 18.06 | N |
| ATOM | 772 | CA | LEU | A1101 | 23.229 | 6.023 | 8.019 | 1.00 | 17.46 | C |
| ATOM | 773 | CB | LEU | A1101 | 22.915 | 7.399 | 7.409 | 1.00 | 15.91 | C |
| ATOM | 774 | CG | LEU | A1101 | 21.475 | 7.611 | 6.919 | 1.00 | 15.54 | C |
| ATOM | 775 | CD1 | LEU | A1101 | 20.510 | 7.621 | 8.101 | 1.00 | 14.01 | C |
| ATOM | 776 | CD2 | LEU | A1101 | 21.380 | 8.931 | 6.150 | 1.00 | 12.69 | C |
| ATOM | 777 | C | LEU | A1101 | 22.561 | 4.945 | 7.190 | 1.00 | 17.42 | C |
| ATOM | 778 | O | LEU | A1101 | 23.124 | 4.436 | 6.218 | 1.00 | 15.25 | O |
| ATOM | 779 | N | SER | A1102 | 21.353 | 4.582 | 7.600 | 1.00 | 17.54 | N |
| ATOM | 780 | CA | SER | A1102 | 20.581 | 3.607 | 6.865 | 1.00 | 15.75 | C |
| ATOM | 781 | CB | SER | A1102 | 20.832 | 2.189 | 7.364 | 1.00 | 16.41 | C |
| ATOM | 782 | OG | SER | A1102 | 20.242 | 1.258 | 6.469 | 1.00 | 14.51 | O |
| ATOM | 783 | C | SER | A1102 | 19.129 | 3.980 | 7.063 | 1.00 | 17.48 | C |
| ATOM | 784 | O | SER | A1102 | 18.711 | 4.322 | 8.173 | 1.00 | 16.20 | O |
| ATOM | 785 | N | ILE | A1103 | 18.376 | 3.940 | 5.968 | 1.00 | 17.02 | N |
| ATOM | 786 | CA | ILE | A1103 | 16.963 | 4.274 | 5.979 | 1.00 | 17.85 | C |
| ATOM | 787 | CB | ILE | A1103 | 16.691 | 5.576 | 5.194 | 1.00 | 19.00 | C |
| ATOM | 788 | CG2 | ILE | A1103 | 15.207 | 5.948 | 5.278 | 1.00 | 18.57 | C |
| ATOM | 789 | CG1 | ILE | A1103 | 17.554 | 6.706 | 5.754 | 1.00 | 18.22 | C |
| ATOM | 790 | CD1 | ILE | A1103 | 17.414 | 8.014 | 4.999 | 1.00 | 20.97 | C |
| ATOM | 791 | C | ILE | A1103 | 16.239 | 3.130 | 5.291 | 1.00 | 17.46 | C |
| ATOM | 792 | O | ILE | A1103 | 16.769 | 2.510 | 4.368 | 1.00 | 17.27 | O |
| ATOM | 793 | N | SER | A1104 | 15.033 | 2.845 | 5.751 | 1.00 | 14.92 | N |
| ATOM | 794 | CA | SER | A1104 | 14.240 | 1.772 | 5.175 | 1.00 | 17.06 | C |
| ATOM | 795 | CB | SER | A1104 | 14.566 | 0.447 | 5.876 | 1.00 | 15.94 | C |
| ATOM | 796 | OG | SER | A1104 | 13.790 | −0.613 | 5.342 | 1.00 | 15.77 | O |
| ATOM | 797 | C | SER | A1104 | 12.779 | 2.155 | 5.384 | 1.00 | 17.20 | C |
| ATOM | 798 | O | SER | A1104 | 12.453 | 2.859 | 6.333 | 1.00 | 15.90 | O |
| ATOM | 799 | N | HIS | A1105 | 11.902 | 1.699 | 4.502 | 1.00 | 18.52 | N |
| ATOM | 800 | CA | HIS | A1105 | 10.494 | 2.048 | 4.619 | 1.00 | 20.22 | C |
| ATOM | 801 | CB | HIS | A1105 | 10.199 | 3.316 | 3.813 | 1.00 | 23.50 | C |
| ATOM | 802 | CG | HIS | A1105 | 10.358 | 3.127 | 2.334 | 1.00 | 25.77 | C |
| ATOM | 803 | CD2 | HIS | A1105 | 9.487 | 2.690 | 1.392 | 1.00 | 26.90 | C |
| ATOM | 804 | ND1 | HIS | A1105 | 11.563 | 3.301 | 1.687 | 1.00 | 26.39 | N |
| ATOM | 805 | CE1 | HIS | A1105 | 11.428 | 2.977 | 0.412 | 1.00 | 27.48 | C |
| ATOM | 806 | NE2 | HIS | A1105 | 10.177 | 2.601 | 0.208 | 1.00 | 27.67 | N |
| ATOM | 807 | C | HIS | A1105 | 9.597 | 0.953 | 4.079 | 1.00 | 21.15 | C |
| ATOM | 808 | O | HIS | A1105 | 10.054 | 0.040 | 3.392 | 1.00 | 21.16 | O |
| ATOM | 809 | N | THR | A1106 | 8.313 | 1.067 | 4.402 | 1.00 | 21.46 | N |
| ATOM | 810 | CA | THR | A1106 | 7.290 | 0.160 | 3.911 | 1.00 | 22.28 | C |
| ATOM | 811 | CB | THR | A1106 | 6.646 | −0.682 | 5.032 | 1.00 | 21.51 | C |
| ATOM | 812 | OG1 | THR | A1106 | 5.891 | 0.169 | 5.904 | 1.00 | 19.68 | O |
| ATOM | 813 | CG2 | THR | A1106 | 7.716 | −1.415 | 5.827 | 1.00 | 21.79 | C |
| ATOM | 814 | C | THR | A1106 | 6.263 | 1.161 | 3.386 | 1.00 | 24.40 | C |
| ATOM | 815 | O | THR | A1106 | 6.560 | 2.356 | 3.309 | 1.00 | 22.60 | O |
| ATOM | 816 | N | ASP | A1107 | 5.070 | 0.698 | 3.032 | 1.00 | 25.68 | N |
| ATOM | 817 | CA | ASP | A1107 | 4.050 | 1.612 | 2.534 | 1.00 | 28.90 | C |
| ATOM | 818 | CB | ASP | A1107 | 2.952 | 0.845 | 1.787 | 1.00 | 34.46 | C |
| ATOM | 819 | CG | ASP | A1107 | 3.479 | 0.098 | 0.574 | 1.00 | 40.33 | C |
| ATOM | 820 | OD1 | ASP | A1107 | 4.229 | 0.704 | −0.225 | 1.00 | 43.51 | O |
| ATOM | 821 | OD2 | ASP | A1107 | 3.134 | −1.093 | 0.413 | 1.00 | 43.99 | O |
| ATOM | 822 | C | ASP | A1107 | 3.420 | 2.385 | 3.691 | 1.00 | 28.81 | C |
| ATOM | 823 | O | ASP | A1107 | 2.613 | 3.285 | 3.470 | 1.00 | 29.78 | O |
| ATOM | 824 | N | GLN | A1108 | 3.801 | 2.049 | 4.921 | 1.00 | 25.83 | N |
| ATOM | 825 | CA | GLN | A1108 | 3.228 | 2.710 | 6.086 | 1.00 | 24.71 | C |
| ATOM | 826 | CB | GLN | A1108 | 2.384 | 1.710 | 6.867 | 1.00 | 27.32 | C |
| ATOM | 827 | CG | GLN | A1108 | 1.328 | 1.026 | 6.030 | 1.00 | 32.60 | C |
| ATOM | 828 | CD | GLN | A1108 | 0.951 | −0.324 | 6.583 | 1.00 | 35.93 | C |
| ATOM | 829 | OE1 | GLN | A1108 | 1.085 | −1.344 | 5.902 | 1.00 | 37.52 | O |
| ATOM | 830 | NE2 | GLN | A1108 | 0.481 | −0.346 | 7.829 | 1.00 | 36.07 | N |
| ATOM | 831 | C | GLN | A1108 | 4.224 | 3.356 | 7.038 | 1.00 | 24.10 | C |
| ATOM | 832 | O | GLN | A1108 | 3.879 | 4.305 | 7.742 | 1.00 | 23.70 | O |
| ATOM | 833 | N | PHE | A1109 | 5.448 | 2.840 | 7.082 | 1.00 | 22.72 | N |
| ATOM | 834 | CA | PHE | A1109 | 6.452 | 3.389 | 7.983 | 1.00 | 21.31 | C |
| ATOM | 835 | CB | PHE | A1109 | 6.719 | 2.446 | 9.164 | 1.00 | 22.89 | C |
| ATOM | 836 | CG | PHE | A1109 | 5.518 | 2.151 | 10.004 | 1.00 | 25.18 | C |
| ATOM | 837 | CD1 | PHE | A1109 | 4.746 | 1.018 | 9.763 | 1.00 | 26.55 | C |
| ATOM | 838 | CD2 | PHE | A1109 | 5.155 | 3.003 | 11.044 | 1.00 | 26.19 | C |
| ATOM | 839 | CE1 | PHE | A1109 | 3.626 | 0.734 | 10.549 | 1.00 | 26.78 | C |
| ATOM | 840 | CE2 | PHE | A1109 | 4.037 | 2.729 | 11.834 | 1.00 | 26.65 | C |
| ATOM | 841 | CZ | PHE | A1109 | 3.273 | 1.591 | 11.584 | 1.00 | 25.88 | C |
| ATOM | 842 | C | PHE | A1109 | 7.785 | 3.630 | 7.318 | 1.00 | 20.22 | C |
| ATOM | 843 | O | PHE | A1109 | 8.096 | 3.053 | 6.275 | 1.00 | 19.27 | O |
| ATOM | 844 | N | VAL | A1110 | 8.573 | 4.489 | 7.951 | 1.00 | 18.43 | N |
| ATOM | 845 | CA | VAL | A1110 | 9.925 | 4.781 | 7.507 | 1.00 | 19.10 | C |
| ATOM | 846 | CB | VAL | A1110 | 10.049 | 6.191 | 6.846 | 1.00 | 19.04 | C |
| ATOM | 847 | CG1 | VAL | A1110 | 9.654 | 7.277 | 7.820 | 1.00 | 19.30 | C |
| ATOM | 848 | CG2 | VAL | A1110 | 11.474 | 6.412 | 6.359 | 1.00 | 23.53 | C |
| ATOM | 849 | C | VAL | A1110 | 10.744 | 4.718 | 8.797 | 1.00 | 19.12 | C |
| ATOM | 850 | O | VAL | A1110 | 10.287 | 5.169 | 9.854 | 1.00 | 17.72 | O |
| ATOM | 851 | N | THR | A1111 | 11.925 | 4.115 | 8.720 | 1.00 | 18.67 | N |
| ATOM | 852 | CA | THR | A1111 | 12.805 | 3.992 | 9.875 | 1.00 | 20.42 | C |
| ATOM | 853 | CB | THR | A1111 | 12.897 | 2.525 | 10.397 | 1.00 | 22.98 | C |
| ATOM | 854 | OG1 | THR | A1111 | 13.363 | 1.670 | 9.345 | 1.00 | 25.86 | O |
| ATOM | 855 | CG2 | THR | A1111 | 11.548 | 2.029 | 10.874 | 1.00 | 24.99 | C |
| ATOM | 856 | C | THR | A1111 | 14.200 | 4.421 | 9.456 | 1.00 | 20.11 | C |
| ATOM | 857 | O | THR | A1111 | 14.575 | 4.287 | 8.291 | 1.00 | 20.53 | O |
| ATOM | 858 | N | ALA | A1112 | 14.962 | 4.946 | 10.404 | 1.00 | 17.15 | N |
| ATOM | 859 | CA | ALA | A1112 | 16.321 | 5.370 | 10.119 | 1.00 | 18.28 | C |
| ATOM | 860 | CB | ALA | A1112 | 16.357 | 6.858 | 9.791 | 1.00 | 16.81 | C |
| ATOM | 861 | C | ALA | A1112 | 17.190 | 5.080 | 11.328 | 1.00 | 18.04 | C |
| ATOM | 862 | O | ALA | A1112 | 16.721 | 5.142 | 12.464 | 1.00 | 17.37 | O |
| ATOM | 863 | N | SER | A1113 | 18.454 | 4.755 | 11.075 | 1.00 | 17.48 | N |
| ATOM | 864 | CA | SER | A1113 | 19.401 | 4.479 | 12.146 | 1.00 | 17.23 | C |
| ATOM | 865 | CB | SER | A1113 | 19.635 | 2.967 | 12.283 | 1.00 | 15.09 | C |
| ATOM | 866 | OG | SER | A1113 | 20.666 | 2.686 | 13.224 | 1.00 | 16.24 | O |
| ATOM | 867 | C | SER | A1113 | 20.718 | 5.187 | 11.844 | 1.00 | 17.94 | C |
| ATOM | 868 | O | SER | A1113 | 21.209 | 5.158 | 10.712 | 1.00 | 18.05 | O |
| ATOM | 869 | N | VAL | A1114 | 21.285 | 5.832 | 12.857 | 1.00 | 18.42 | N |
| ATOM | 870 | CA | VAL | A1114 | 22.546 | 6.543 | 12.683 | 1.00 | 17.97 | C |
| ATOM | 871 | CB | VAL | A1114 | 22.350 | 8.082 | 12.783 | 1.00 | 17.35 | C |
| ATOM | 872 | CG1 | VAL | A1114 | 23.708 | 8.784 | 12.874 | 1.00 | 15.58 | C |
| ATOM | 873 | CG2 | VAL | A1114 | 21.571 | 8.592 | 11.571 | 1.00 | 16.65 | C |
| ATOM | 874 | C | VAL | A1114 | 23.565 | 6.130 | 13.735 | 1.00 | 18.00 | C |
| ATOM | 875 | O | VAL | A1114 | 23.241 | 6.023 | 14.926 | 1.00 | 17.56 | O |
| ATOM | 876 | N | ILE | A1115 | 24.793 | 5.885 | 13.286 | 1.00 | 17.57 | N |
| ATOM | 877 | CA | ILE | A1115 | 25.885 | 5.536 | 14.181 | 1.00 | 15.61 | C |
| ATOM | 878 | CB | ILE | A1115 | 26.416 | 4.109 | 13.951 | 1.00 | 17.48 | C |
| ATOM | 879 | CG2 | ILE | A1115 | 27.514 | 3.806 | 14.965 | 1.00 | 14.97 | C |
| ATOM | 880 | CG1 | ILE | A1115 | 25.287 | 3.086 | 14.082 | 1.00 | 16.92 | C |
| ATOM | 881 | CD1 | ILE | A1115 | 25.695 | 1.685 | 13.638 | 1.00 | 16.69 | C |
| ATOM | 882 | C | ILE | A1115 | 27.019 | 6.499 | 13.860 | 1.00 | 17.74 | C |
| ATOM | 883 | O | ILE | A1115 | 27.470 | 6.573 | 12.714 | 1.00 | 16.21 | O |
| ATOM | 884 | N | LEU | A1116 | 27.470 | 7.246 | 14.864 | 1.00 | 17.78 | N |
| ATOM | 885 | CA | LEU | A1116 | 28.562 | 8.188 | 14.668 | 1.00 | 19.32 | C |
| ATOM | 886 | CB | LEU | A1116 | 28.333 | 9.442 | 15.510 | 1.00 | 20.38 | C |
| ATOM | 887 | CG | LEU | A1116 | 26.963 | 10.095 | 15.322 | 1.00 | 20.47 | C |
| ATOM | 888 | CD1 | LEU | A1116 | 26.877 | 11.326 | 16.205 | 1.00 | 22.94 | C |
| ATOM | 889 | CD2 | LEU | A1116 | 26.751 | 10.462 | 13.860 | 1.00 | 18.60 | C |
| ATOM | 890 | C | LEU | A1116 | 29.851 | 7.490 | 15.093 | 1.00 | 21.10 | C |
| ATOM | 891 | O | LEU | A1116 | 29.909 | 6.870 | 16.156 | 1.00 | 19.16 | O |
| ATOM | 892 | N | GLU | A1117 | 30.880 | 7.590 | 14.259 | 1.00 | 22.54 | N |
| ATOM | 893 | CA | GLU | A1117 | 32.141 | 6.934 | 14.554 | 1.00 | 25.40 | C |
| ATOM | 894 | CB | GLU | A1117 | 32.323 | 5.743 | 13.616 | 1.00 | 25.16 | C |
| ATOM | 895 | CG | GLU | A1117 | 33.629 | 5.000 | 13.817 | 1.00 | 27.61 | C |
| ATOM | 896 | CD | GLU | A1117 | 33.720 | 3.772 | 12.950 | 1.00 | 27.04 | C |
| ATOM | 897 | OE1 | GLU | A1117 | 33.391 | 3.875 | 11.750 | 1.00 | 27.90 | O |
| ATOM | 898 | OE2 | GLU | A1117 | 34.126 | 2.712 | 13.465 | 1.00 | 27.49 | O |
| ATOM | 899 | C | GLU | A1117 | 33.358 | 7.852 | 14.464 | 1.00 | 27.82 | C |
| ATOM | 900 | O | GLU | A1117 | 33.425 | 8.742 | 13.616 | 1.00 | 26.44 | O |
| ATOM | 901 | N | GLU | A1118 | 34.328 | 7.605 | 15.339 | 1.00 | 31.38 | N |
| ATOM | 902 | CA | GLU | A1118 | 35.549 | 8.396 | 15.387 | 1.00 | 35.38 | C |
| ATOM | 903 | CB | GLU | A1118 | 35.632 | 9.122 | 16.732 | 1.00 | 38.96 | C |
| ATOM | 904 | CG | GLU | A1118 | 36.450 | 10.397 | 16.711 | 1.00 | 44.28 | C |
| ATOM | 905 | CD | GLU | A1118 | 35.953 | 11.381 | 15.671 | 1.00 | 46.94 | C |
| ATOM | 906 | OE1 | GLU | A1118 | 36.206 | 11.153 | 14.469 | 1.00 | 47.99 | O |
| ATOM | 907 | OE2 | GLU | A1118 | 35.302 | 12.377 | 16.054 | 1.00 | 49.20 | O |
| ATOM | 908 | C | GLU | A1118 | 36.756 | 7.482 | 15.210 | 1.00 | 36.85 | C |
| ATOM | 909 | O | GLU | A1118 | 37.586 | 7.767 | 14.319 | 1.00 | 39.18 | O |
| ATOM | 910 | OXT | GLU | A1118 | 36.858 | 6.488 | 15.967 | 1.00 | 37.85 | O |
| TER | 911 | | GLU | A1118 | | | | | | |
| ATOM | 912 | CB | MET | B2003 | 36.089 | −4.001 | 20.131 | 1.00 | 36.93 | C |
| ATOM | 913 | CG | MET | B2003 | 35.997 | −4.450 | 21.581 | 1.00 | 42.07 | C |
| ATOM | 914 | SD | MET | B2003 | 37.062 | −5.865 | 21.964 | 1.00 | 49.34 | S |
| ATOM | 915 | CE | MET | B2003 | 36.267 | −7.158 | 21.006 | 1.00 | 46.76 | C |
| ATOM | 916 | C | MET | B2003 | 33.796 | −3.017 | 20.233 | 1.00 | 31.67 | C |
| ATOM | 917 | O | MET | B2003 | 33.469 | −2.921 | 21.422 | 1.00 | 28.54 | O |
| ATOM | 918 | N | MET | B2003 | 35.809 | −1.560 | 20.439 | 1.00 | 33.64 | N |
| ATOM | 919 | CA | MET | B2003 | 35.239 | −2.773 | 19.791 | 1.00 | 33.04 | C |
| ATOM | 920 | N | ILE | B2004 | 32.938 | −3.327 | 19.264 | 1.00 | 29.22 | N |
| ATOM | 921 | CA | ILE | B2004 | 31.537 | −3.604 | 19.538 | 1.00 | 26.95 | C |
| ATOM | 922 | CB | ILE | B2004 | 30.694 | −3.586 | 18.236 | 1.00 | 27.19 | C |
| ATOM | 923 | CG2 | ILE | B2004 | 29.273 | −4.070 | 18.528 | 1.00 | 26.67 | C |
| ATOM | 924 | CG1 | ILE | B2004 | 30.692 | −2.175 | 17.642 | 1.00 | 27.75 | C |
| ATOM | 925 | CD1 | ILE | B2004 | 29.965 | −2.048 | 16.307 | 1.00 | 28.82 | C |
| ATOM | 926 | C | ILE | B2004 | 31.452 | −4.986 | 20.165 | 1.00 | 25.64 | C |
| ATOM | 927 | O | ILE | B2004 | 32.092 | −5.924 | 19.693 | 1.00 | 26.78 | O |
| ATOM | 928 | N | VAL | B2005 | 30.681 | −5.114 | 21.240 | 1.00 | 23.42 | N |
| ATOM | 929 | CA | VAL | B2005 | 30.534 | −6.406 | 21.904 | 1.00 | 22.67 | C |
| ATOM | 930 | CB | VAL | B2005 | 31.099 | −6.379 | 23.344 | 1.00 | 23.16 | C |
| ATOM | 931 | CG1 | VAL | B2005 | 32.615 | −6.311 | 23.305 | 1.00 | 22.80 | C |
| ATOM | 932 | CG2 | VAL | B2005 | 30.552 | −5.197 | 24.096 | 1.00 | 23.38 | C |
| ATOM | 933 | C | VAL | B2005 | 29.086 | −6.878 | 21.948 | 1.00 | 21.18 | C |
| ATOM | 934 | O | VAL | B2005 | 28.772 | −7.884 | 22.577 | 1.00 | 23.33 | O |
| ATOM | 935 | N | GLY | B2006 | 28.200 | −6.155 | 21.275 | 1.00 | 19.21 | N |
| ATOM | 936 | CA | GLY | B2006 | 26.811 | −6.568 | 21.260 | 1.00 | 17.80 | C |
| ATOM | 937 | C | GLY | B2006 | 25.896 | −5.584 | 20.567 | 1.00 | 17.03 | C |
| ATOM | 938 | O | GLY | B2006 | 26.161 | −4.387 | 20.553 | 1.00 | 16.68 | O |
| ATOM | 939 | N | HIS | B2007 | 24.813 | −6.093 | 19.989 | 1.00 | 16.35 | N |
| ATOM | 940 | CA | HIS | B2007 | 23.846 | −5.237 | 19.323 | 1.00 | 16.88 | C |
| ATOM | 941 | CB | HIS | B2007 | 24.216 | −5.042 | 17.847 | 1.00 | 17.35 | C |
| ATOM | 942 | CG | HIS | B2007 | 23.254 | −4.170 | 17.097 | 1.00 | 18.35 | C |
| ATOM | 943 | CD2 | HIS | B2007 | 22.786 | −4.239 | 15.827 | 1.00 | 17.84 | C |
| ATOM | 944 | ND1 | HIS | B2007 | 22.668 | −3.058 | 17.660 | 1.00 | 18.04 | N |
| ATOM | 945 | CE1 | HIS | B2007 | 21.880 | −2.479 | 16.769 | 1.00 | 19.69 | C |
| ATOM | 946 | NE2 | HIS | B2007 | 21.934 | −3.176 | 15.648 | 1.00 | 16.84 | N |
| ATOM | 947 | C | HIS | B2007 | 22.447 | −5.831 | 19.444 | 1.00 | 17.26 | C |
| ATOM | 948 | O | HIS | B2007 | 22.238 | −7.027 | 19.213 | 1.00 | 14.74 | O |
| ATOM | 949 | N | GLY | B2008 | 21.490 | −4.990 | 19.819 | 1.00 | 16.95 | N |
| ATOM | 950 | CA | GLY | B2008 | 20.129 | −5.464 | 19.962 | 1.00 | 17.68 | C |
| ATOM | 951 | C | GLY | B2008 | 19.095 | −4.401 | 19.661 | 1.00 | 18.32 | C |
| ATOM | 952 | O | GLY | B2008 | 19.321 | −3.212 | 19.901 | 1.00 | 19.75 | O |
| ATOM | 953 | N | ILE | B2009 | 17.964 | −4.830 | 19.112 | 1.00 | 17.31 | N |
| ATOM | 954 | CA | ILE | B2009 | 16.868 | −3.917 | 18.821 | 1.00 | 18.07 | C |
| ATOM | 955 | CB | ILE | B2009 | 16.727 | −3.598 | 17.306 | 1.00 | 16.33 | C |
| ATOM | 956 | CG2 | ILE | B2009 | 18.025 | −2.990 | 16.777 | 1.00 | 16.15 | C |
| ATOM | 957 | CG1 | ILE | B2009 | 16.361 | −4.856 | 16.519 | 1.00 | 19.06 | C |
| ATOM | 958 | CD1 | ILE | B2009 | 16.014 | −4.559 | 15.060 | 1.00 | 16.01 | C |
| ATOM | 959 | C | ILE | B2009 | 15.607 | −4.607 | 19.312 | 1.00 | 17.52 | C |
| ATOM | 960 | O | ILE | B2009 | 15.576 | −5.827 | 19.448 | 1.00 | 17.73 | O |
| ATOM | 961 | N | ASP | B2010 | 14.580 | −3.825 | 19.607 | 1.00 | 18.54 | N |
| ATOM | 962 | CA | ASP | B2010 | 13.327 | −4.384 | 20.089 | 1.00 | 20.76 | C |
| ATOM | 963 | CB | ASP | B2010 | 13.401 | −4.663 | 21.598 | 1.00 | 21.38 | C |
| ATOM | 964 | CG | ASP | B2010 | 12.157 | −5.372 | 22.127 | 1.00 | 22.62 | C |
| ATOM | 965 | OD1 | ASP | B2010 | 11.403 | −4.762 | 22.915 | 1.00 | 22.62 | O |
| ATOM | 966 | OD2 | ASP | B2010 | 11.932 | −6.544 | 21.756 | 1.00 | 23.17 | O |
| ATOM | 967 | C | ASP | B2010 | 12.198 | −3.416 | 19.814 | 1.00 | 21.83 | C |
| ATOM | 968 | O | ASP | B2010 | 12.378 | −2.200 | 19.912 | 1.00 | 21.02 | O |
| ATOM | 969 | N | ILE | B2011 | 11.039 | −3.966 | 19.464 | 1.00 | 21.90 | N |
| ATOM | 970 | CA | ILE | B2011 | 9.848 | −3.163 | 19.189 | 1.00 | 25.52 | C |
| ATOM | 971 | CB | ILE | B2011 | 9.346 | −3.346 | 17.752 | 1.00 | 27.88 | C |
| ATOM | 972 | CG2 | ILE | B2011 | 8.410 | −2.194 | 17.388 | 1.00 | 30.28 | C |
| ATOM | 973 | CG1 | ILE | B2011 | 10.518 | −3.410 | 16.783 | 1.00 | 29.66 | C |
| ATOM | 974 | CD1 | ILE | B2011 | 10.131 | −3.937 | 15.420 | 1.00 | 31.53 | C |
| ATOM | 975 | C | ILE | B2011 | 8.738 | −3.670 | 20.102 | 1.00 | 24.08 | C |
| ATOM | 976 | O | ILE | B2011 | 8.561 | −4.874 | 20.258 | 1.00 | 22.40 | O |
| ATOM | 977 | N | GLU | B2012 | 8.002 | −2.757 | 20.718 | 1.00 | 24.39 | N |
| ATOM | 978 | CA | GLU | B2012 | 6.911 | −3.156 | 21.598 | 1.00 | 24.77 | C |
| ATOM | 979 | CB | GLU | B2012 | 7.269 | −2.868 | 23.056 | 1.00 | 25.42 | C |
| ATOM | 980 | CG | GLU | B2012 | 8.482 | −3.637 | 23.554 | 1.00 | 28.89 | C |
| ATOM | 981 | CD | GLU | B2012 | 8.233 | −5.139 | 23.652 | 1.00 | 31.05 | C |
| ATOM | 982 | OE1 | GLU | B2012 | 9.224 | −5.895 | 23.721 | 1.00 | 31.07 | O |
| ATOM | 983 | OE2 | GLU | B2012 | 7.053 | −5.560 | 23.671 | 1.00 | 30.63 | O |
| ATOM | 984 | C | GLU | B2012 | 5.655 | −2.387 | 21.208 | 1.00 | 24.28 | C |
| ATOM | 985 | O | GLU | B2012 | 5.732 | −1.235 | 20.786 | 1.00 | 24.45 | O |
| ATOM | 986 | N | GLU | B2013 | 4.507 | −3.043 | 21.328 | 1.00 | 22.06 | N |
| ATOM | 987 | CA | GLU | B2013 | 3.223 | −2.431 | 21.008 | 1.00 | 21.01 | C |
| ATOM | 988 | CB | GLU | B2013 | 2.241 | −3.508 | 20.545 | 1.00 | 21.98 | C |
| ATOM | 989 | CG | GLU | B2013 | 0.857 | −3.010 | 20.179 | 0.05 | 20.80 | C |
| ATOM | 990 | CD | GLU | B2013 | −0.106 | −4.152 | 19.921 | 0.05 | 20.56 | C |
| ATOM | 991 | OE1 | GLU | B2013 | 0.231 | −5.039 | 19.109 | 0.05 | 20.26 | O |
| ATOM | 992 | OE2 | GLU | B2013 | −1.196 | −4.166 | 20.529 | 0.05 | 20.00 | O |
| ATOM | 993 | C | GLU | B2013 | 2.719 | −1.793 | 22.292 | 1.00 | 20.42 | C |
| ATOM | 994 | O | GLU | B2013 | 2.644 | −2.457 | 23.326 | 1.00 | 19.82 | O |
| ATOM | 995 | N | LEU | B2014 | 2.383 | −0.512 | 22.244 | 1.00 | 20.95 | N |
| ATOM | 996 | CA | LEU | B2014 | 1.906 | 0.149 | 23.452 | 1.00 | 25.05 | C |
| ATOM | 997 | CB | LEU | B2014 | 1.530 | 1.604 | 23.162 | 1.00 | 26.46 | C |
| ATOM | 998 | CG | LEU | B2014 | 2.610 | 2.478 | 22.521 | 1.00 | 29.15 | C |
| ATOM | 999 | CD1 | LEU | B2014 | 2.171 | 3.929 | 22.610 | 1.00 | 32.28 | C |
| ATOM | 1000 | CD2 | LEU | B2014 | 3.945 | 2.294 | 23.225 | 1.00 | 28.39 | C |
| ATOM | 1001 | C | LEU | B2014 | 0.708 | −0.582 | 24.053 | 1.00 | 25.52 | C |
| ATOM | 1002 | O | LEU | B2014 | 0.635 | −0.773 | 25.266 | 1.00 | 25.48 | O |
| ATOM | 1003 | N | ALA | B2015 | −0.220 | −1.001 | 23.196 | 1.00 | 26.92 | N |
| ATOM | 1004 | CA | ALA | B2015 | −1.425 | −1.697 | 23.637 | 1.00 | 28.21 | C |
| ATOM | 1005 | CB | ALA | B2015 | −2.267 | −2.088 | 22.426 | 1.00 | 29.71 | C |
| ATOM | 1006 | C | ALA | B2015 | −1.144 | −2.932 | 24.490 | 1.00 | 29.10 | C |
| ATOM | 1007 | O | ALA | B2015 | −1.864 | −3.208 | 25.450 | 1.00 | 29.79 | O |
| ATOM | 1008 | N | SER | B2016 | −0.106 | −3.681 | 24.135 | 1.00 | 29.36 | N |
| ATOM | 1009 | CA | SER | B2016 | 0.246 | −4.885 | 24.875 | 1.00 | 30.31 | C |
| ATOM | 1010 | CB | SER | B2016 | 1.357 | −5.633 | 24.147 | 1.00 | 32.28 | C |
| ATOM | 1011 | OG | SER | B2016 | 1.032 | −5.782 | 22.779 | 1.00 | 33.64 | O |
| ATOM | 1012 | C | SER | B2016 | 0.705 | −4.508 | 26.275 | 1.00 | 30.37 | C |
| ATOM | 1013 | O | SER | B2016 | 0.369 | −5.170 | 27.260 | 1.00 | 29.37 | O |
| ATOM | 1014 | N | ILE | B2017 | 1.476 | −3.431 | 26.353 | 1.00 | 30.04 | N |
| ATOM | 1015 | CA | ILE | B2017 | 1.979 | −2.955 | 27.625 | 1.00 | 30.17 | C |
| ATOM | 1016 | CB | ILE | B2017 | 2.907 | −1.741 | 27.420 | 1.00 | 29.67 | C |
| ATOM | 1017 | CG2 | ILE | B2017 | 3.323 | −1.153 | 28.765 | 1.00 | 31.54 | C |
| ATOM | 1018 | CG1 | ILE | B2017 | 4.141 | −2.178 | 26.630 | 1.00 | 29.63 | C |
| ATOM | 1019 | CD1 | ILE | B2017 | 4.883 | −3.368 | 27.246 | 1.00 | 29.53 | C |
| ATOM | 1020 | C | ILE | B2017 | 0.814 | −2.585 | 28.537 | 1.00 | 30.53 | C |
| ATOM | 1021 | O | ILE | B2017 | 0.736 | −3.051 | 29.673 | 1.00 | 29.90 | O |
| ATOM | 1022 | N | GLU | B2018 | −0.100 | −1.762 | 28.034 | 1.00 | 31.91 | N |
| ATOM | 1023 | CA | GLU | B2018 | −1.252 | −1.363 | 28.831 | 1.00 | 34.92 | C |
| ATOM | 1024 | CB | GLU | B2018 | −2.113 | −0.355 | 28.064 | 1.00 | 37.97 | C |
| ATOM | 1025 | CG | GLU | B2018 | −1.895 | −0.370 | 26.565 | 1.00 | 42.86 | C |
| ATOM | 1026 | CD | GLU | B2018 | −2.698 | 0.700 | 25.844 | 1.00 | 45.97 | C |
| ATOM | 1027 | OE1 | GLU | B2018 | −3.945 | 0.608 | 25.843 | 1.00 | 47.86 | O |
| ATOM | 1028 | OE2 | GLU | B2018 | −2.084 | 1.633 | 25.279 | 1.00 | 47.02 | O |
| ATOM | 1029 | C | GLU | B2018 | −2.085 | −2.573 | 29.247 | 1.00 | 34.56 | C |
| ATOM | 1030 | O | GLU | B2018 | −2.490 | −2.678 | 30.404 | 1.00 | 35.89 | O |
| ATOM | 1031 | N | SER | B2019 | −2.349 | −3.488 | 28.321 | 1.00 | 33.29 | N |
| ATOM | 1032 | CA | SER | B2019 | −3.117 | −4.674 | 28.681 | 1.00 | 33.75 | C |
| ATOM | 1033 | CB | SER | B2019 | −3.270 | −5.623 | 27.490 | 1.00 | 32.73 | C |
| ATOM | 1034 | OG | SER | B2019 | −4.314 | −5.203 | 26.635 | 1.00 | 36.89 | O |
| ATOM | 1035 | C | SER | B2019 | −2.416 | −5.402 | 29.822 | 1.00 | 33.12 | C |
| ATOM | 1036 | O | SER | B2019 | −3.066 | −5.963 | 30.698 | 1.00 | 32.83 | O |
| ATOM | 1037 | N | ALA | B2020 | −1.087 | −5.398 | 29.808 | 1.00 | 33.22 | N |
| ATOM | 1038 | CA | ALA | B2020 | −0.329 | −6.055 | 30.869 | 1.00 | 35.36 | C |
| ATOM | 1039 | CB | ALA | B2020 | 1.157 | −6.105 | 30.510 | 1.00 | 34.87 | C |
| ATOM | 1040 | C | ALA | B2020 | −0.537 | −5.295 | 32.179 | 1.00 | 36.08 | C |
| ATOM | 1041 | O | ALA | B2020 | −0.669 | −5.898 | 33.241 | 1.00 | 34.70 | O |
| ATOM | 1042 | N | VAL | B2021 | −0.570 | −3.968 | 32.097 | 1.00 | 38.89 | N |
| ATOM | 1043 | CA | VAL | B2021 | −0.783 | −3.133 | 33.277 | 1.00 | 42.15 | C |
| ATOM | 1044 | CB | VAL | B2021 | −0.672 | −1.627 | 32.931 | 1.00 | 42.05 | C |
| ATOM | 1045 | CG1 | VAL | B2021 | −1.060 | −0.775 | 34.138 | 1.00 | 42.48 | C |
| ATOM | 1046 | CG2 | VAL | B2021 | 0.746 | −1.298 | 32.504 | 1.00 | 42.42 | C |
| ATOM | 1047 | C | VAL | B2021 | −2.176 | −3.410 | 33.841 | 1.00 | 44.98 | C |
| ATOM | 1048 | O | VAL | B2021 | −2.491 | −3.029 | 34.967 | 1.00 | 45.67 | O |
| ATOM | 1049 | N | THR | B2022 | −3.001 | −4.085 | 33.047 | 1.00 | 47.50 | N |
| ATOM | 1050 | CA | THR | B2022 | −4.359 | −4.420 | 33.451 | 1.00 | 50.10 | C |
| ATOM | 1051 | CB | THR | B2022 | −5.323 | −4.368 | 32.236 | 1.00 | 51.16 | C |
| ATOM | 1052 | OG1 | THR | B2022 | −5.501 | −3.007 | 31.824 | 1.00 | 53.24 | O |
| ATOM | 1053 | CG2 | THR | B2022 | −6.677 | −4.957 | 32.592 | 1.00 | 52.95 | C |
| ATOM | 1054 | C | THR | B2022 | −4.454 | −5.799 | 34.109 | 1.00 | 50.81 | C |
| ATOM | 1055 | O | THR | B2022 | −4.937 | −5.918 | 35.232 | 1.00 | 51.36 | O |
| ATOM | 1056 | N | ARG | B2023 | −3.988 | −6.836 | 33.421 | 1.00 | 51.25 | N |
| ATOM | 1057 | CA | ARG | B2023 | −4.064 | −8.187 | 33.968 | 1.00 | 52.22 | C |
| ATOM | 1058 | CB | ARG | B2023 | −3.746 | −9.220 | 32.883 | 1.00 | 52.29 | C |
| ATOM | 1059 | CG | ARG | B2023 | −4.157 | −10.644 | 33.242 | 0.05 | 51.97 | C |
| ATOM | 1060 | CD | ARG | B2023 | −5.531 | −10.663 | 33.905 | 0.05 | 51.83 | C |
| ATOM | 1061 | NE | ARG | B2023 | −6.183 | −11.966 | 33.828 | 0.05 | 51.66 | N |
| ATOM | 1062 | CZ | ARG | B2023 | −6.697 | −12.474 | 32.713 | 0.05 | 51.57 | C |
| ATOM | 1063 | NH1 | ARG | B2023 | −6.634 | −11.789 | 31.579 | 0.05 | 51.47 | N |
| ATOM | 1064 | NH2 | ARG | B2023 | −7.284 | −13.663 | 32.730 | 0.05 | 51.49 | N |
| ATOM | 1065 | C | ARG | B2023 | −3.157 | −8.405 | 35.173 | 1.00 | 53.22 | C |
| ATOM | 1066 | O | ARG | B2023 | −3.256 | −9.424 | 35.859 | 1.00 | 53.07 | O |
| ATOM | 1067 | N | HIS | B2024 | −2.274 | −7.447 | 35.432 | 1.00 | 53.34 | N |
| ATOM | 1068 | CA | HIS | B2024 | −1.365 | −7.544 | 36.564 | 1.00 | 53.62 | C |
| ATOM | 1069 | CB | HIS | B2024 | −0.042 | −8.176 | 36.127 | 1.00 | 54.97 | C |
| ATOM | 1070 | CG | HIS | B2024 | −0.208 | −9.477 | 35.403 | 1.00 | 56.91 | C |
| ATOM | 1071 | CD2 | HIS | B2024 | −0.006 | −10.755 | 35.805 | 1.00 | 57.80 | C |
| ATOM | 1072 | ND1 | HIS | B2024 | −0.660 | −9.552 | 34.103 | 1.00 | 57.75 | N |
| ATOM | 1073 | CE1 | HIS | B2024 | −0.729 | −10.820 | 33.735 | 1.00 | 58.06 | C |
| ATOM | 1074 | NE2 | HIS | B2024 | −0.338 | −11.570 | 34.750 | 1.00 | 57.59 | N |
| ATOM | 1075 | C | HIS | B2024 | −1.127 | −6.157 | 37.148 | 1.00 | 53.35 | C |
| ATOM | 1076 | O | HIS | B2024 | −0.312 | −5.385 | 36.644 | 1.00 | 53.29 | O |
| ATOM | 1077 | N | GLU | B2025 | −1.861 | −5.854 | 38.214 | 1.00 | 52.80 | N |
| ATOM | 1078 | CA | GLU | B2025 | −1.785 | −4.569 | 38.905 | 1.00 | 51.46 | C |
| ATOM | 1079 | CB | GLU | B2025 | −2.618 | −4.637 | 40.188 | 1.00 | 54.49 | C |
| ATOM | 1080 | CG | GLU | B2025 | −4.095 | −4.920 | 39.949 | 1.00 | 56.85 | C |
| ATOM | 1081 | CD | GLU | B2025 | −4.798 | −5.434 | 41.190 | 1.00 | 58.84 | C |
| ATOM | 1082 | OE1 | GLU | B2025 | −4.520 | −6.584 | 41.595 | 1.00 | 60.08 | O |
| ATOM | 1083 | OE2 | GLU | B2025 | −5.624 | −4.690 | 41.763 | 1.00 | 59.42 | O |
| ATOM | 1084 | C | GLU | B2025 | −0.364 | −4.122 | 39.247 | 1.00 | 48.83 | C |
| ATOM | 1085 | O | GLU | B2025 | −0.058 | −2.931 | 39.211 | 1.00 | 48.34 | O |
| ATOM | 1086 | N | GLY | B2026 | 0.497 | −5.074 | 39.588 | 1.00 | 45.49 | N |
| ATOM | 1087 | CA | GLY | B2026 | 1.864 | −4.729 | 39.932 | 1.00 | 40.95 | C |
| ATOM | 1088 | C | GLY | B2026 | 2.826 | −4.920 | 38.774 | 1.00 | 37.63 | C |
| ATOM | 1089 | O | GLY | B2026 | 4.027 | −5.077 | 38.978 | 1.00 | 37.06 | O |
| ATOM | 1090 | N | PHE | B2027 | 2.301 | −4.902 | 37.554 | 1.00 | 33.22 | N |
| ATOM | 1091 | CA | PHE | B2027 | 3.135 | −5.083 | 36.374 | 1.00 | 30.35 | C |
| ATOM | 1092 | CB | PHE | B2027 | 2.287 | −4.995 | 35.105 | 1.00 | 29.47 | C |
| ATOM | 1093 | CG | PHE | B2027 | 3.079 | −5.162 | 33.840 | 1.00 | 28.37 | C |
| ATOM | 1094 | CD1 | PHE | B2027 | 3.501 | −6.421 | 33.431 | 1.00 | 28.75 | C |
| ATOM | 1095 | CD2 | PHE | B2027 | 3.432 | −4.055 | 33.076 | 1.00 | 26.82 | C |
| ATOM | 1096 | CE1 | PHE | B2027 | 4.264 | −6.575 | 32.277 | 1.00 | 27.85 | C |
| ATOM | 1097 | CE2 | PHE | B2027 | 4.195 | −4.199 | 31.919 | 1.00 | 27.50 | C |
| ATOM | 1098 | CZ | PHE | B2027 | 4.611 | −5.458 | 31.520 | 1.00 | 28.11 | C |
| ATOM | 1099 | C | PHE | B2027 | 4.245 | −4.038 | 36.303 | 1.00 | 28.35 | C |
| ATOM | 1100 | O | PHE | B2027 | 5.419 | −4.378 | 36.179 | 1.00 | 27.55 | O |
| ATOM | 1101 | N | ALA | B2028 | 3.863 | −2.768 | 36.376 | 1.00 | 26.54 | N |
| ATOM | 1102 | CA | ALA | B2028 | 4.820 | −1.674 | 36.306 | 1.00 | 26.97 | C |
| ATOM | 1103 | CB | ALA | B2028 | 4.103 | −0.340 | 36.503 | 1.00 | 26.06 | C |
| ATOM | 1104 | C | ALA | B2028 | 5.938 | −1.815 | 37.329 | 1.00 | 27.20 | C |
| ATOM | 1105 | O | ALA | B2028 | 7.112 | −1.697 | 36.998 | 1.00 | 26.33 | O |
| ATOM | 1106 | N | LYS | B2029 | 5.555 | −2.075 | 38.572 | 1.00 | 28.14 | N |
| ATOM | 1107 | CA | LYS | B2029 | 6.496 | −2.213 | 39.673 | 1.00 | 30.24 | C |
| ATOM | 1108 | CB | LYS | B2029 | 5.738 | −2.543 | 40.965 | 1.00 | 33.94 | C |
| ATOM | 1109 | CG | LYS | B2029 | 6.597 | −2.502 | 42.215 | 1.00 | 39.10 | C |
| ATOM | 1110 | CD | LYS | B2029 | 7.038 | −1.082 | 42.540 | 1.00 | 42.39 | C |
| ATOM | 1111 | CE | LYS | B2029 | 8.108 | −1.070 | 43.629 | 1.00 | 45.14 | C |
| ATOM | 1112 | NZ | LYS | B2029 | 7.712 | −1.858 | 44.836 | 1.00 | 46.16 | N |
| ATOM | 1113 | C | LYS | B2029 | 7.567 | −3.264 | 39.437 | 1.00 | 28.56 | C |
| ATOM | 1114 | O | LYS | B2029 | 8.696 | −3.114 | 39.902 | 1.00 | 28.42 | O |
| ATOM | 1115 | N | ARG | B2030 | 7.219 | −4.325 | 38.717 | 1.00 | 26.96 | N |
| ATOM | 1116 | CA | ARG | B2030 | 8.171 | −5.397 | 38.452 | 1.00 | 25.76 | C |
| ATOM | 1117 | CB | ARG | B2030 | 7.430 | −6.708 | 38.180 | 1.00 | 27.87 | C |
| ATOM | 1118 | CG | ARG | B2030 | 6.318 | −7.007 | 39.174 | 0.05 | 26.73 | C |
| ATOM | 1119 | CD | ARG | B2030 | 6.358 | −8.451 | 39.652 | 0.05 | 26.68 | C |
| ATOM | 1120 | NE | ARG | B2030 | 6.295 | −9.416 | 38.557 | 0.05 | 26.45 | N |
| ATOM | 1121 | CZ | ARG | B2030 | 5.263 | −9.553 | 37.730 | 0.05 | 26.36 | C |
| ATOM | 1122 | NH1 | ARG | B2030 | 4.191 | −8.783 | 37.862 | 0.05 | 26.22 | N |
| ATOM | 1123 | NH2 | ARG | B2030 | 5.301 | −10.469 | 36.771 | 0.05 | 26.29 | N |
| ATOM | 1124 | C | ARG | B2030 | 9.069 | −5.076 | 37.266 | 1.00 | 24.16 | C |
| ATOM | 1125 | O | ARG | B2030 | 10.200 | −5.551 | 37.187 | 1.00 | 22.83 | O |
| ATOM | 1126 | N | VAL | B2031 | 8.559 | −4.270 | 36.341 | 1.00 | 22.75 | N |
| ATOM | 1127 | CA | VAL | B2031 | 9.319 | −3.903 | 35.158 | 1.00 | 22.54 | C |
| ATOM | 1128 | CB | VAL | B2031 | 8.371 | −3.536 | 33.984 | 1.00 | 23.94 | C |
| ATOM | 1129 | CG1 | VAL | B2031 | 9.156 | −2.884 | 32.855 | 1.00 | 24.54 | C |
| ATOM | 1130 | CG2 | VAL | B2031 | 7.677 | −4.794 | 33.467 | 1.00 | 24.38 | C |
| ATOM | 1131 | C | VAL | B2031 | 10.264 | −2.739 | 35.409 | 1.00 | 21.20 | C |
| ATOM | 1132 | O | VAL | B2031 | 11.364 | −2.699 | 34.858 | 1.00 | 21.89 | O |
| ATOM | 1133 | N | LEU | B2032 | 9.837 | −1.805 | 36.252 | 1.00 | 20.43 | N |
| ATOM | 1134 | CA | LEU | B2032 | 10.623 | −0.607 | 36.552 | 1.00 | 21.46 | C |
| ATOM | 1135 | CB | LEU | B2032 | 9.722 | 0.624 | 36.459 | 1.00 | 21.16 | C |
| ATOM | 1136 | CG | LEU | B2032 | 8.902 | 0.814 | 35.180 | 1.00 | 22.44 | C |
| ATOM | 1137 | CD1 | LEU | B2032 | 7.913 | 1.951 | 35.383 | 1.00 | 25.05 | C |
| ATOM | 1138 | CD2 | LEU | B2032 | 9.822 | 1.107 | 34.003 | 1.00 | 23.51 | C |
| ATOM | 1139 | C | LEU | B2032 | 11.289 | −0.613 | 37.928 | 1.00 | 20.88 | C |
| ATOM | 1140 | O | LEU | B2032 | 10.758 | −1.188 | 38.875 | 1.00 | 24.06 | O |
| ATOM | 1141 | N | THR | B2033 | 12.451 | 0.027 | 38.035 | 1.00 | 20.86 | N |
| ATOM | 1142 | CA | THR | B2033 | 13.147 | 0.117 | 39.317 | 1.00 | 20.81 | C |
| ATOM | 1143 | CB | THR | B2033 | 14.602 | 0.569 | 39.160 | 1.00 | 20.39 | C |
| ATOM | 1144 | OG1 | THR | B2033 | 14.623 | 1.853 | 38.521 | 1.00 | 21.21 | O |
| ATOM | 1145 | CG2 | THR | B2033 | 15.394 | −0.443 | 38.341 | 1.00 | 20.57 | C |
| ATOM | 1146 | C | THR | B2033 | 12.432 | 1.189 | 40.119 | 1.00 | 21.74 | C |
| ATOM | 1147 | O | THR | B2033 | 11.479 | 1.806 | 39.635 | 1.00 | 22.52 | O |
| ATOM | 1148 | N | ALA | B2034 | 12.893 | 1.427 | 41.339 | 1.00 | 22.64 | N |
| ATOM | 1149 | CA | ALA | B2034 | 12.267 | 2.441 | 42.177 | 1.00 | 24.18 | C |
| ATOM | 1150 | CB | ALA | B2034 | 12.939 | 2.479 | 43.541 | 1.00 | 24.55 | C |
| ATOM | 1151 | C | ALA | B2034 | 12.371 | 3.806 | 41.502 | 1.00 | 25.08 | C |
| ATOM | 1152 | O | ALA | B2034 | 11.393 | 4.555 | 41.437 | 1.00 | 24.57 | O |
| ATOM | 1153 | N | LEU | B2035 | 13.558 | 4.119 | 40.989 | 1.00 | 25.67 | N |
| ATOM | 1154 | CA | LEU | B2035 | 13.786 | 5.403 | 40.337 | 1.00 | 27.37 | C |
| ATOM | 1155 | CB | LEU | B2035 | 15.274 | 5.576 | 40.025 | 1.00 | 30.75 | C |
| ATOM | 1156 | CG | LEU | B2035 | 15.792 | 7.017 | 40.016 | 1.00 | 34.25 | C |
| ATOM | 1157 | CD1 | LEU | B2035 | 15.529 | 7.681 | 41.373 | 1.00 | 34.25 | C |
| ATOM | 1158 | CD2 | LEU | B2035 | 17.286 | 7.010 | 39.715 | 1.00 | 36.03 | C |
| ATOM | 1159 | C | LEU | B2035 | 12.948 | 5.550 | 39.066 | 1.00 | 26.19 | C |
| ATOM | 1160 | O | LEU | B2035 | 12.424 | 6.625 | 38.789 | 1.00 | 25.81 | O |
| ATOM | 1161 | N | GLU | B2036 | 12.829 | 4.479 | 38.288 | 1.00 | 24.86 | N |
| ATOM | 1162 | CA | GLU | B2036 | 12.010 | 4.523 | 37.074 | 1.00 | 24.94 | C |
| ATOM | 1163 | CB | GLU | B2036 | 12.218 | 3.256 | 36.238 | 1.00 | 24.20 | C |
| ATOM | 1164 | CG | GLU | B2036 | 13.521 | 3.220 | 35.439 | 1.00 | 22.34 | C |
| ATOM | 1165 | CD | GLU | B2036 | 13.823 | 1.834 | 34.878 | 1.00 | 22.43 | C |
| ATOM | 1166 | OE1 | GLU | B2036 | 14.592 | 1.744 | 33.899 | 1.00 | 20.15 | O |
| ATOM | 1167 | OE2 | GLU | B2036 | 13.302 | 0.834 | 35.425 | 1.00 | 21.31 | O |
| ATOM | 1168 | C | GLU | B2036 | 10.528 | 4.645 | 37.461 | 1.00 | 25.14 | C |
| ATOM | 1169 | O | GLU | B2036 | 9.740 | 5.261 | 36.746 | 1.00 | 24.49 | O |
| ATOM | 1170 | N | MET | B2037 | 10.155 | 4.051 | 38.593 | 1.00 | 25.36 | N |
| ATOM | 1171 | CA | MET | B2037 | 8.772 | 4.116 | 39.067 | 1.00 | 27.03 | C |
| ATOM | 1172 | CB | MET | B2037 | 8.570 | 3.202 | 40.279 | 1.00 | 28.19 | C |
| ATOM | 1173 | CG | MET | B2037 | 8.077 | 1.811 | 39.932 | 1.00 | 30.50 | C |
| ATOM | 1174 | SD | MET | B2037 | 6.592 | 1.878 | 38.903 | 1.00 | 35.36 | S |
| ATOM | 1175 | CE | MET | B2037 | 5.315 | 2.130 | 40.111 | 1.00 | 31.11 | C |
| ATOM | 1176 | C | MET | B2037 | 8.389 | 5.541 | 39.445 | 1.00 | 27.51 | C |
| ATOM | 1177 | O | MET | B2037 | 7.288 | 6.004 | 39.146 | 1.00 | 27.72 | O |
| ATOM | 1178 | N | GLU | B2038 | 9.309 | 6.232 | 40.104 | 1.00 | 29.26 | N |
| ATOM | 1179 | CA | GLU | B2038 | 9.075 | 7.604 | 40.522 | 1.00 | 30.50 | C |
| ATOM | 1180 | CB | GLU | B2038 | 10.261 | 8.089 | 41.363 | 1.00 | 33.66 | C |
| ATOM | 1181 | CG | GLU | B2038 | 10.100 | 7.747 | 42.852 | 1.00 | 38.27 | C |
| ATOM | 1182 | CD | GLU | B2038 | 11.384 | 7.278 | 43.517 | 1.00 | 41.20 | C |
| ATOM | 1183 | OE1 | GLU | B2038 | 12.448 | 7.886 | 43.264 | 1.00 | 41.95 | O |
| ATOM | 1184 | OE2 | GLU | B2038 | 11.322 | 6.307 | 44.309 | 1.00 | 42.56 | O |
| ATOM | 1185 | C | GLU | B2038 | 8.827 | 8.512 | 39.320 | 1.00 | 29.98 | C |
| ATOM | 1186 | O | GLU | B2038 | 8.059 | 9.473 | 39.403 | 1.00 | 29.40 | O |
| ATOM | 1187 | N | ARG | B2039 | 9.465 | 8.198 | 38.198 | 1.00 | 28.37 | N |
| ATOM | 1188 | CA | ARG | B2039 | 9.272 | 8.973 | 36.986 | 1.00 | 28.60 | C |
| ATOM | 1189 | CB | ARG | B2039 | 10.374 | 8.670 | 35.973 | 1.00 | 32.59 | C |
| ATOM | 1190 | CG | ARG | B2039 | 10.103 | 9.236 | 34.589 | 1.00 | 36.59 | C |
| ATOM | 1191 | CD | ARG | B2039 | 9.892 | 10.740 | 34.627 | 1.00 | 42.20 | C |
| ATOM | 1192 | NE | ARG | B2039 | 9.750 | 11.301 | 33.284 | 1.00 | 46.45 | N |
| ATOM | 1193 | CZ | ARG | B2039 | 9.599 | 12.597 | 33.025 | 1.00 | 48.88 | C |
| ATOM | 1194 | NH1 | ARG | B2039 | 9.477 | 13.014 | 31.770 | 1.00 | 48.84 | N |
| ATOM | 1195 | NH2 | ARG | B2039 | 9.570 | 13.478 | 34.018 | 1.00 | 49.51 | N |
| ATOM | 1196 | C | ARG | B2039 | 7.926 | 8.585 | 36.397 | 1.00 | 27.40 | C |
| ATOM | 1197 | O | ARG | B2039 | 7.107 | 9.439 | 36.047 | 1.00 | 28.21 | O |
| ATOM | 1198 | N | PHE | B2040 | 7.717 | 7.280 | 36.291 | 1.00 | 25.73 | N |
| ATOM | 1199 | CA | PHE | B2040 | 6.489 | 6.710 | 35.758 | 1.00 | 26.22 | C |
| ATOM | 1200 | CB | PHE | B2040 | 6.546 | 5.185 | 35.916 | 1.00 | 24.31 | C |
| ATOM | 1201 | CG | PHE | B2040 | 5.265 | 4.477 | 35.573 | 1.00 | 26.63 | C |
| ATOM | 1202 | CD1 | PHE | B2040 | 4.871 | 4.318 | 34.248 | 1.00 | 26.24 | C |
| ATOM | 1203 | CD2 | PHE | B2040 | 4.467 | 3.938 | 36.579 | 1.00 | 26.94 | C |
| ATOM | 1204 | CE1 | PHE | B2040 | 3.698 | 3.627 | 33.930 | 1.00 | 27.58 | C |
| ATOM | 1205 | CE2 | PHE | B2040 | 3.297 | 3.249 | 36.276 | 1.00 | 27.41 | C |
| ATOM | 1206 | CZ | PHE | B2040 | 2.911 | 3.090 | 34.950 | 1.00 | 27.69 | C |
| ATOM | 1207 | C | PHE | B2040 | 5.245 | 7.262 | 36.463 | 1.00 | 26.33 | C |
| ATOM | 1208 | O | PHE | B2040 | 4.269 | 7.622 | 35.814 | 1.00 | 27.93 | O |
| ATOM | 1209 | N | THR | B2041 | 5.287 | 7.324 | 37.792 | 1.00 | 27.19 | N |
| ATOM | 1210 | CA | THR | B2041 | 4.151 | 7.805 | 38.575 | 1.00 | 27.42 | C |
| ATOM | 1211 | CB | THR | B2041 | 4.328 | 7.485 | 40.078 | 1.00 | 27.34 | C |
| ATOM | 1212 | OG1 | THR | B2041 | 5.504 | 8.132 | 40.577 | 1.00 | 28.52 | O |
| ATOM | 1213 | CG2 | THR | B2041 | 4.458 | 5.986 | 40.294 | 1.00 | 28.04 | C |
| ATOM | 1214 | C | THR | B2041 | 3.896 | 9.304 | 38.432 | 1.00 | 28.31 | C |
| ATOM | 1215 | O | THR | B2041 | 2.840 | 9.796 | 38.840 | 1.00 | 27.03 | O |
| ATOM | 1216 | N | SER | B2042 | 4.854 | 10.024 | 37.853 | 1.00 | 28.11 | N |
| ATOM | 1217 | CA | SER | B2042 | 4.706 | 11.465 | 37.665 | 1.00 | 31.34 | C |
| ATOM | 1218 | CB | SER | B2042 | 6.039 | 12.179 | 37.896 | 1.00 | 32.90 | C |
| ATOM | 1219 | OG | SER | B2042 | 6.326 | 12.278 | 39.282 | 1.00 | 36.54 | O |
| ATOM | 1220 | C | SER | B2042 | 4.168 | 11.841 | 36.289 | 1.00 | 31.32 | C |
| ATOM | 1221 | O | SER | B2042 | 3.909 | 13.012 | 36.018 | 1.00 | 30.89 | O |
| ATOM | 1222 | N | LEU | B2043 | 4.008 | 10.849 | 35.421 | 1.00 | 31.83 | N |
| ATOM | 1223 | CA | LEU | B2043 | 3.487 | 11.088 | 34.077 | 1.00 | 31.92 | C |
| ATOM | 1224 | CB | LEU | B2043 | 4.293 | 10.300 | 33.038 | 1.00 | 29.48 | C |
| ATOM | 1225 | CG | LEU | B2043 | 5.785 | 10.603 | 32.912 | 1.00 | 29.62 | C |
| ATOM | 1226 | CD1 | LEU | B2043 | 6.420 | 9.633 | 31.921 | 1.00 | 29.54 | C |
| ATOM | 1227 | CD2 | LEU | B2043 | 5.985 | 12.035 | 32.462 | 1.00 | 29.33 | C |
| ATOM | 1228 | C | LEU | B2043 | 2.039 | 10.623 | 34.058 | 1.00 | 33.01 | C |
| ATOM | 1229 | O | LEU | B2043 | 1.569 | 10.030 | 35.026 | 1.00 | 32.48 | O |
| ATOM | 1230 | N | LYS | B2044 | 1.337 | 10.877 | 32.957 | 1.00 | 36.01 | N |
| ATOM | 1231 | CA | LYS | B2044 | −0.060 | 10.469 | 32.850 | 1.00 | 38.06 | C |
| ATOM | 1232 | CB | LYS | B2044 | −0.967 | 11.586 | 33.375 | 1.00 | 39.86 | C |
| ATOM | 1233 | CG | LYS | B2044 | −2.402 | 11.149 | 33.619 | 1.00 | 43.44 | C |
| ATOM | 1234 | CD | LYS | B2044 | −3.295 | 12.317 | 34.013 | 1.00 | 45.82 | C |
| ATOM | 1235 | CE | LYS | B2044 | −4.630 | 11.825 | 34.554 | 1.00 | 46.21 | C |
| ATOM | 1236 | NZ | LYS | B2044 | −5.292 | 10.879 | 33.615 | 1.00 | 47.83 | N |
| ATOM | 1237 | C | LYS | B2044 | −0.472 | 10.116 | 31.422 | 1.00 | 38.81 | C |
| ATOM | 1238 | O | LYS | B2044 | 0.225 | 10.438 | 30.459 | 1.00 | 39.23 | O |
| ATOM | 1239 | N | GLY | B2045 | −1.611 | 9.443 | 31.299 | 1.00 | 39.57 | N |
| ATOM | 1240 | CA | GLY | B2045 | −2.134 | 9.068 | 29.996 | 1.00 | 40.10 | C |
| ATOM | 1241 | C | GLY | B2045 | −1.177 | 8.355 | 29.062 | 1.00 | 40.74 | C |
| ATOM | 1242 | O | GLY | B2045 | −0.513 | 7.394 | 29.454 | 1.00 | 41.23 | O |
| ATOM | 1243 | N | ARG | B2046 | −1.115 | 8.832 | 27.820 | 1.00 | 41.17 | N |
| ATOM | 1244 | CA | ARG | B2046 | −0.255 | 8.249 | 26.791 | 1.00 | 41.02 | C |
| ATOM | 1245 | CB | ARG | B2046 | −0.396 | 9.015 | 25.472 | 1.00 | 43.31 | C |
| ATOM | 1246 | CG | ARG | B2046 | −1.812 | 9.081 | 24.931 | 1.00 | 47.77 | C |
| ATOM | 1247 | CD | ARG | B2046 | −1.882 | 9.873 | 23.627 | 1.00 | 50.51 | C |
| ATOM | 1248 | NE | ARG | B2046 | −3.260 | 10.180 | 23.244 | 1.00 | 52.23 | N |
| ATOM | 1249 | CZ | ARG | B2046 | −3.611 | 10.768 | 22.103 | 1.00 | 52.56 | C |
| ATOM | 1250 | NH1 | ARG | B2046 | −4.890 | 11.012 | 21.844 | 1.00 | 52.50 | N |
| ATOM | 1251 | NH2 | ARG | B2046 | −2.684 | 11.107 | 21.217 | 1.00 | 53.75 | N |
| ATOM | 1252 | C | ARG | B2046 | 1.212 | 8.229 | 27.184 | 1.00 | 39.59 | C |
| ATOM | 1253 | O | ARG | B2046 | 1.873 | 7.198 | 27.078 | 1.00 | 38.95 | O |
| ATOM | 1254 | N | ARG | B2047 | 1.718 | 9.375 | 27.631 | 1.00 | 38.59 | N |
| ATOM | 1255 | CA | ARG | B2047 | 3.117 | 9.492 | 28.022 | 1.00 | 36.93 | C |
| ATOM | 1256 | CB | ARG | B2047 | 3.415 | 10.914 | 28.497 | 1.00 | 39.53 | C |
| ATOM | 1257 | CG | ARG | B2047 | 3.284 | 11.965 | 27.408 | 1.00 | 43.89 | C |
| ATOM | 1258 | CD | ARG | B2047 | 3.915 | 13.275 | 27.843 | 1.00 | 46.98 | C |
| ATOM | 1259 | NE | ARG | B2047 | 5.322 | 13.091 | 28.189 | 1.00 | 49.81 | N |
| ATOM | 1260 | CZ | ARG | B2047 | 6.145 | 14.072 | 28.545 | 1.00 | 50.90 | C |
| ATOM | 1261 | NH1 | ARG | B2047 | 5.708 | 15.324 | 28.603 | 1.00 | 51.65 | N |
| ATOM | 1262 | NH2 | ARG | B2047 | 7.409 | 13.800 | 28.845 | 1.00 | 52.13 | N |
| ATOM | 1263 | C | ARG | B2047 | 3.543 | 8.496 | 29.095 | 1.00 | 34.22 | C |
| ATOM | 1264 | O | ARG | B2047 | 4.671 | 8.017 | 29.086 | 1.00 | 33.86 | O |
| ATOM | 1265 | N | GLN | B2048 | 2.646 | 8.187 | 30.022 | 1.00 | 31.68 | N |
| ATOM | 1266 | CA | GLN | B2048 | 2.968 | 7.247 | 31.081 | 1.00 | 29.06 | C |
| ATOM | 1267 | CB | GLN | B2048 | 1.844 | 7.212 | 32.121 | 1.00 | 29.98 | C |
| ATOM | 1268 | CG | GLN | B2048 | 2.236 | 6.524 | 33.420 | 1.00 | 32.80 | C |
| ATOM | 1269 | CD | GLN | B2048 | 1.089 | 6.443 | 34.415 | 1.00 | 34.55 | C |
| ATOM | 1270 | OE1 | GLN | B2048 | 1.297 | 6.158 | 35.596 | 1.00 | 36.97 | O |
| ATOM | 1271 | NE2 | GLN | B2048 | −0.125 | 6.682 | 33.940 | 1.00 | 33.10 | N |
| ATOM | 1272 | C | GLN | B2048 | 3.161 | 5.861 | 30.471 | 1.00 | 27.54 | C |
| ATOM | 1273 | O | GLN | B2048 | 4.134 | 5.170 | 30.769 | 1.00 | 26.96 | O |
| ATOM | 1274 | N | ILE | B2049 | 2.230 | 5.459 | 29.612 | 1.00 | 25.61 | N |
| ATOM | 1275 | CA | ILE | B2049 | 2.318 | 4.159 | 28.961 | 1.00 | 24.60 | C |
| ATOM | 1276 | CB | ILE | B2049 | 1.099 | 3.894 | 28.053 | 1.00 | 24.71 | C |
| ATOM | 1277 | CG2 | ILE | B2049 | 1.310 | 2.606 | 27.254 | 1.00 | 25.39 | C |
| ATOM | 1278 | CG1 | ILE | B2049 | −0.174 | 3.814 | 28.906 | 1.00 | 25.99 | C |
| ATOM | 1279 | CD1 | ILE | B2049 | −0.111 | 2.776 | 30.004 | 1.00 | 26.01 | C |
| ATOM | 1280 | C | ILE | B2049 | 3.570 | 4.090 | 28.104 | 1.00 | 23.58 | C |
| ATOM | 1281 | O | ILE | B2049 | 4.281 | 3.091 | 28.120 | 1.00 | 24.35 | O |
| ATOM | 1282 | N | GLU | B2050 | 3.828 | 5.160 | 27.360 | 1.00 | 22.52 | N |
| ATOM | 1283 | CA | GLU | B2050 | 4.985 | 5.238 | 26.482 | 1.00 | 23.92 | C |
| ATOM | 1284 | CB | GLU | B2050 | 4.980 | 6.575 | 25.739 | 1.00 | 24.81 | C |
| ATOM | 1285 | CG | GLU | B2050 | 6.216 | 6.842 | 24.901 | 1.00 | 27.69 | C |
| ATOM | 1286 | CD | GLU | B2050 | 6.470 | 5.767 | 23.859 | 1.00 | 30.29 | C |
| ATOM | 1287 | OE1 | GLU | B2050 | 5.514 | 5.384 | 23.149 | 1.00 | 32.11 | O |
| ATOM | 1288 | OE2 | GLU | B2050 | 7.632 | 5.314 | 23.740 | 1.00 | 29.75 | O |
| ATOM | 1289 | C | GLU | B2050 | 6.278 | 5.085 | 27.270 | 1.00 | 23.59 | C |
| ATOM | 1290 | O | GLU | B2050 | 7.235 | 4.452 | 26.804 | 1.00 | 23.37 | O |
| ATOM | 1291 | N | TYR | B2051 | 6.304 | 5.666 | 28.466 | 1.00 | 23.27 | N |
| ATOM | 1292 | CA | TYR | B2051 | 7.487 | 5.584 | 29.306 | 1.00 | 23.17 | C |
| ATOM | 1293 | CB | TYR | B2051 | 7.300 | 6.397 | 30.592 | 1.00 | 22.74 | C |
| ATOM | 1294 | CG | TYR | B2051 | 8.520 | 6.367 | 31.492 | 1.00 | 22.74 | C |
| ATOM | 1295 | CD1 | TYR | B2051 | 8.667 | 5.382 | 32.474 | 1.00 | 21.76 | C |
| ATOM | 1296 | CE1 | TYR | B2051 | 9.818 | 5.317 | 33.263 | 1.00 | 23.29 | C |
| ATOM | 1297 | CD2 | TYR | B2051 | 9.555 | 7.288 | 31.324 | 1.00 | 21.56 | C |
| ATOM | 1298 | CE2 | TYR | B2051 | 10.708 | 7.230 | 32.106 | 1.00 | 22.47 | C |
| ATOM | 1299 | CZ | TYR | B2051 | 10.832 | 6.243 | 33.072 | 1.00 | 24.14 | C |
| ATOM | 1300 | OH | TYR | B2051 | 11.973 | 6.182 | 33.843 | 1.00 | 26.11 | O |
| ATOM | 1301 | C | TYR | B2051 | 7.749 | 4.123 | 29.645 | 1.00 | 22.35 | C |
| ATOM | 1302 | O | TYR | B2051 | 8.834 | 3.603 | 29.397 | 1.00 | 21.78 | O |
| ATOM | 1303 | N | LEU | B2052 | 6.735 | 3.470 | 30.200 | 1.00 | 21.05 | N |
| ATOM | 1304 | CA | LEU | B2052 | 6.820 | 2.069 | 30.581 | 1.00 | 20.91 | C |
| ATOM | 1305 | CB | LEU | B2052 | 5.486 | 1.626 | 31.193 | 1.00 | 19.71 | C |
| ATOM | 1306 | CG | LEU | B2052 | 5.321 | 0.141 | 31.535 | 1.00 | 20.97 | C |
| ATOM | 1307 | CD1 | LEU | B2052 | 6.463 | −0.293 | 32.453 | 1.00 | 20.68 | C |
| ATOM | 1308 | CD2 | LEU | B2052 | 3.953 | −0.092 | 32.206 | 1.00 | 18.56 | C |
| ATOM | 1309 | C | LEU | B2052 | 7.176 | 1.164 | 29.394 | 1.00 | 19.19 | C |
| ATOM | 1310 | O | LEU | B2052 | 8.055 | 0.305 | 29.502 | 1.00 | 18.65 | O |
| ATOM | 1311 | N | ALA | B2053 | 6.501 | 1.365 | 28.264 | 1.00 | 18.96 | N |
| ATOM | 1312 | CA | ALA | B2053 | 6.748 | 0.553 | 27.075 | 1.00 | 18.97 | C |
| ATOM | 1313 | CB | ALA | B2053 | 5.712 | 0.867 | 26.003 | 1.00 | 19.24 | C |
| ATOM | 1314 | C | ALA | B2053 | 8.153 | 0.777 | 26.526 | 1.00 | 19.65 | C |
| ATOM | 1315 | O | ALA | B2053 | 8.811 | −0.167 | 26.067 | 1.00 | 19.77 | O |
| ATOM | 1316 | N | GLY | B2054 | 8.604 | 2.028 | 26.569 | 1.00 | 18.25 | N |
| ATOM | 1317 | CA | GLY | B2054 | 9.930 | 2.355 | 26.084 | 1.00 | 16.89 | C |
| ATOM | 1318 | C | GLY | B2054 | 11.004 | 1.748 | 26.970 | 1.00 | 18.42 | C |
| ATOM | 1319 | O | GLY | B2054 | 12.072 | 1.344 | 26.488 | 1.00 | 17.94 | O |
| ATOM | 1320 | N | ARG | B2055 | 10.731 | 1.692 | 28.270 | 1.00 | 16.40 | N |
| ATOM | 1321 | CA | ARG | B2055 | 11.684 | 1.112 | 29.202 | 1.00 | 19.09 | C |
| ATOM | 1322 | CB | ARG | B2055 | 11.253 | −1.377 | 30.647 | 1.00 | 20.94 | C |
| ATOM | 1323 | CG | ARG | B2055 | 12.060 | 2.478 | 31.329 | 1.00 | 20.89 | C |
| ATOM | 1324 | CD | ARG | B2055 | 11.968 | 3.790 | 30.570 | 1.00 | 24.51 | C |
| ATOM | 1325 | NE | ARG | B2055 | 12.987 | 4.745 | 31.003 | 1.00 | 25.33 | N |
| ATOM | 1326 | CZ | ARG | B2055 | 13.239 | 5.897 | 30.388 | 1.00 | 25.95 | C |
| ATOM | 1327 | NH1 | ARG | B2055 | 12.544 | 6.243 | 29.313 | 1.00 | 23.97 | N |
| ATOM | 1328 | NH2 | ARG | B2055 | 14.194 | 6.700 | 30.843 | 1.00 | 25.82 | N |
| ATOM | 1329 | C | ARG | B2055 | 11.774 | −0.386 | 28.932 | 1.00 | 18.98 | C |
| ATOM | 1330 | O | ARG | B2055 | 12.855 | −0.971 | 28.985 | 1.00 | 18.90 | O |
| ATOM | 1331 | N | TRP | B2056 | 10.632 | −1.003 | 28.631 | 1.00 | 20.10 | N |
| ATOM | 1332 | CA | TRP | B2056 | 10.613 | −2.426 | 28.322 | 1.00 | 20.52 | C |
| ATOM | 1333 | CB | TRP | B2056 | 9.178 | −2.930 | 28.107 | 1.00 | 22.74 | C |
| ATOM | 1334 | CG | TRP | B2056 | 9.118 | −4.394 | 27.738 | 1.00 | 26.17 | C |
| ATOM | 1335 | CD2 | TRP | B2056 | 8.573 | −5.459 | 28.527 | 1.00 | 27.99 | C |
| ATOM | 1336 | CE2 | TRP | B2056 | 8.791 | −6.661 | 27.815 | 1.00 | 28.39 | C |
| ATOM | 1337 | CE3 | TRP | B2056 | 7.924 | −5.516 | 29.767 | 1.00 | 27.60 | C |
| ATOM | 1338 | CD1 | TRP | B2056 | 9.623 | −4.978 | 26.607 | 1.00 | 26.87 | C |
| ATOM | 1339 | NE1 | TRP | B2056 | 9.432 | −6.337 | 26.648 | 1.00 | 29.27 | N |
| ATOM | 1340 | CZ2 | TRP | B2056 | 8.384 | −7.906 | 28.301 | 1.00 | 29.62 | C |
| ATOM | 1341 | CZ3 | TRP | B2056 | 7.516 | −6.760 | 30.253 | 1.00 | 30.44 | C |
| ATOM | 1342 | CH2 | TRP | B2056 | 7.750 | −7.937 | 29.519 | 1.00 | 30.93 | C |
| ATOM | 1343 | C | TRP | B2056 | 11.430 | −2.659 | 27.056 | 1.00 | 18.69 | C |
| ATOM | 1344 | O | TRP | B2056 | 12.265 | −3.562 | 27.007 | 1.00 | 15.35 | O |
| ATOM | 1345 | N | SER | B2057 | 11.190 | −1.843 | 26.032 | 1.00 | 18.10 | N |
| ATOM | 1346 | CA | SER | B2057 | 11.925 | −1.993 | 24.778 | 1.00 | 19.73 | C |
| ATOM | 1347 | CB | SER | B2057 | 11.475 | −0.948 | 23.756 | 1.00 | 21.79 | C |
| ATOM | 1348 | OG | SER | B2057 | 10.174 | −1.245 | 23.273 | 1.00 | 30.44 | O |
| ATOM | 1349 | C | SER | B2057 | 13.427 | −1.857 | 25.009 | 1.00 | 17.21 | C |
| ATOM | 1350 | O | SER | B2057 | 14.207 | −2.679 | 24.539 | 1.00 | 17.96 | O |
| ATOM | 1351 | N | ALA | B2058 | 13.821 | −0.814 | 25.728 | 1.00 | 15.24 | N |
| ATOM | 1352 | CA | ALA | B2058 | 15.233 | −0.568 | 26.016 | 1.00 | 15.12 | C |
| ATOM | 1353 | CB | ALA | B2058 | 15.387 | 0.718 | 26.819 | 1.00 | 14.34 | C |
| ATOM | 1354 | C | ALA | B2058 | 15.879 | −1.724 | 26.768 | 1.00 | 15.45 | C |
| ATOM | 1355 | O | ALA | B2058 | 16.981 | −2.158 | 26.421 | 1.00 | 14.80 | O |
| ATOM | 1356 | N | LYS | B2059 | 15.203 | −2.216 | 27.804 | 1.00 | 15.14 | N |
| ATOM | 1357 | CA | LYS | B2059 | 15.748 | −3.315 | 28.593 | 1.00 | 16.97 | C |
| ATOM | 1358 | CB | LYS | B2059 | 14.909 | −3.523 | 29.860 | 1.00 | 18.38 | C |
| ATOM | 1359 | CG | LYS | B2059 | 14.958 | −2.303 | 30.796 | 1.00 | 17.80 | C |
| ATOM | 1360 | CD | LYS | B2059 | 14.216 | −2.546 | 32.097 | 1.00 | 19.77 | C |
| ATOM | 1361 | CE | LYS | B2059 | 14.345 | −1.327 | 33.021 | 1.00 | 19.57 | C |
| ATOM | 1362 | NZ | LYS | B2059 | 13.932 | −1.624 | 34.417 | 1.00 | 20.44 | N |
| ATOM | 1363 | C | LYS | B2059 | 15.857 | −4.608 | 27.792 | 1.00 | 17.95 | C |
| ATOM | 1364 | O | LYS | B2059 | 16.805 | −5.375 | 27.967 | 1.00 | 16.15 | O |
| ATOM | 1365 | N | GLU | B2060 | 14.897 | −4.844 | 26.904 | 1.00 | 17.88 | N |
| ATOM | 1366 | CA | GLU | B2060 | 14.923 | −6.041 | 26.070 | 1.00 | 17.87 | C |
| ATOM | 1367 | CB | GLU | B2060 | 13.551 | −6.261 | 25.420 | 1.00 | 19.58 | C |
| ATOM | 1368 | CG | GLU | B2060 | 13.519 | −7.359 | 24.361 | 1.00 | 23.62 | C |
| ATOM | 1369 | CD | GLU | B2060 | 13.834 | −8.753 | 24.897 | 1.00 | 24.96 | C |
| ATOM | 1370 | OE1 | GLU | B2060 | 14.150 | −8.906 | 26.099 | 1.00 | 27.91 | O |
| ATOM | 1371 | OE2 | GLU | B2060 | 13.764 | −9.707 | 24.096 | 1.00 | 27.02 | O |
| ATOM | 1372 | C | GLU | B2060 | 16.013 | −5.885 | 24.999 | 1.00 | 17.82 | C |
| ATOM | 1373 | O | GLU | B2060 | 16.769 | −6.816 | 24.741 | 1.00 | 16.78 | O |
| ATOM | 1374 | N | ALA | B2061 | 16.106 | −4.700 | 24.396 | 1.00 | 15.53 | N |
| ATOM | 1375 | CA | ALA | B2061 | 17.126 | −4.443 | 23.379 | 1.00 | 15.22 | C |
| ATOM | 1376 | CB | ALA | B2061 | 17.017 | −3.010 | 22.868 | 1.00 | 12.83 | C |
| ATOM | 1377 | C | ALA | B2061 | 18.514 | −4.683 | 23.984 | 1.00 | 15.45 | C |
| ATOM | 1378 | O | ALA | B2061 | 19.396 | −5.252 | 23.337 | 1.00 | 15.13 | O |
| ATOM | 1379 | N | PHE | B2062 | 18.699 | −4.248 | 25.227 | 1.00 | 15.78 | N |
| ATOM | 1380 | CA | PHE | B2062 | 19.965 | −4.441 | 25.923 | 1.00 | 16.16 | C |
| ATOM | 1381 | CB | PHE | B2062 | 19.972 | −3.664 | 27.244 | 1.00 | 18.34 | C |
| ATOM | 1382 | CG | PHE | B2062 | 21.173 | −3.954 | 28.110 | 1.00 | 20.05 | C |
| ATOM | 1383 | CD1 | PHE | B2062 | 21.180 | −5.055 | 28.972 | 1.00 | 20.65 | C |
| ATOM | 1384 | CD2 | PHE | B2062 | 22.311 | −3.158 | 28.030 | 1.00 | 20.37 | C |
| ATOM | 1385 | CE1 | PHE | B2062 | 22.304 | −5.358 | 29.737 | 1.00 | 20.45 | C |
| ATOM | 1386 | CE2 | PHE | B2062 | 23.450 | −3.453 | 28.796 | 1.00 | 21.28 | C |
| ATOM | 1387 | CZ | PHE | B2062 | 23.443 | −4.552 | 29.647 | 1.00 | 20.21 | C |
| ATOM | 1388 | C | PHE | B2062 | 20.210 | −5.930 | 26.195 | 1.00 | 17.69 | C |
| ATOM | 1389 | O | PHE | B2062 | 21.297 | −6.455 | 25.933 | 1.00 | 17.00 | O |
| ATOM | 1390 | N | SER | B2063 | 19.203 | −6.608 | 26.730 | 1.00 | 18.58 | N |
| ATOM | 1391 | CA | SER | B2063 | 19.336 | −8.033 | 27.016 | 1.00 | 19.54 | C |
| ATOM | 1392 | CB | SER | B2063 | 18.025 | −8.584 | 27.565 | 1.00 | 19.74 | C |
| ATOM | 1393 | OG | SER | B2063 | 17.710 | −7.950 | 28.793 | 1.00 | 22.28 | O |
| ATOM | 1394 | C | SER | B2063 | 19.733 | −8.789 | 25.753 | 1.00 | 20.63 | C |
| ATOM | 1395 | O | SER | B2063 | 20.574 | −9.682 | 25.803 | 1.00 | 18.71 | O |
| ATOM | 1396 | N | LYS | B2064 | 19.128 | −8.430 | 24.620 | 1.00 | 19.41 | N |
| ATOM | 1397 | CA | LYS | B2064 | 19.457 | −9.088 | 23.362 | 1.00 | 20.24 | C |
| ATOM | 1398 | CB | LYS | B2064 | 18.447 | −8.705 | 22.276 | 1.00 | 21.74 | C |
| ATOM | 1399 | CG | LYS | B2064 | 17.036 | −9.246 | 22.553 | 1.00 | 21.35 | C |
| ATOM | 1400 | CD | LYS | B2064 | 16.007 | −8.785 | 21.525 | 1.00 | 21.53 | C |
| ATOM | 1401 | CE | LYS | B2064 | 16.378 | −9.233 | 20.116 | 1.00 | 19.60 | C |
| ATOM | 1402 | NZ | LYS | B2064 | 15.400 | −8.764 | 19.091 | 1.00 | 19.68 | N |
| ATOM | 1403 | C | LYS | B2064 | 20.875 | −8.709 | 22.946 | 1.00 | 20.67 | C |
| ATOM | 1404 | O | LYS | B2064 | 21.573 | −9.498 | 22.310 | 1.00 | 21.49 | O |
| ATOM | 1405 | N | ALA | B2065 | 21.303 | −7.502 | 23.308 | 1.00 | 17.85 | N |
| ATOM | 1406 | CA | ALA | B2065 | 22.660 | −7.059 | 22.995 | 1.00 | 19.57 | C |
| ATOM | 1407 | CB | ALA | B2065 | 22.826 | −5.576 | 23.323 | 1.00 | 15.70 | C |
| ATOM | 1408 | C | ALA | B2065 | 23.647 | −7.892 | 23.820 | 1.00 | 20.80 | C |
| ATOM | 1409 | O | ALA | B2065 | 24.793 | −8.096 | 23.414 | 1.00 | 18.12 | O |
| ATOM | 1410 | N | MET | B2066 | 23.199 | −8.359 | 24.985 | 1.00 | 22.49 | N |
| ATOM | 1411 | CA | MET | B2066 | 24.039 | −9.187 | 25.849 | 1.00 | 27.36 | C |
| ATOM | 1412 | CB | MET | B2066 | 23.608 | −9.069 | 27.310 | 1.00 | 27.69 | C |
| ATOM | 1413 | CG | MET | B2066 | 23.933 | −7.749 | 27.971 | 1.00 | 30.93 | C |
| ATOM | 1414 | SD | MET | B2066 | 25.678 | −7.548 | 28.342 | 1.00 | 35.00 | S |
| ATOM | 1415 | CE | MET | B2066 | 25.840 | −8.691 | 29.727 | 1.00 | 35.63 | C |
| ATOM | 1416 | C | MET | B2066 | 23.927 | −10.646 | 25.416 | 1.00 | 28.77 | C |
| ATOM | 1417 | O | MET | B2066 | 24.570 | −11.517 | 25.990 | 1.00 | 29.95 | O |
| ATOM | 1418 | N | GLY | B2067 | 23.095 | −10.906 | 24.411 | 1.00 | 30.61 | N |
| ATOM | 1419 | CA | GLY | B2067 | 22.927 | −12.266 | 23.924 | 1.00 | 32.14 | C |
| ATOM | 1420 | C | GLY | B2067 | 21.816 | −13.073 | 24.577 | 1.00 | 33.83 | C |
| ATOM | 1421 | O | GLY | B2067 | 21.728 | −14.282 | 24.353 | 1.00 | 35.17 | O |
| ATOM | 1422 | N | THR | B2068 | 20.964 | −12.425 | 25.369 | 1.00 | 33.49 | N |
| ATOM | 1423 | CA | THR | B2068 | 19.870 | −13.124 | 26.048 | 1.00 | 35.61 | C |
| ATOM | 1424 | CB | THR | B2068 | 20.225 | −13.393 | 27.528 | 1.00 | 37.06 | C |
| ATOM | 1425 | OG1 | THR | B2068 | 20.480 | −12.149 | 28.194 | 1.00 | 37.62 | O |
| ATOM | 1426 | CG2 | THR | B2068 | 21.461 | −14.274 | 27.622 | 1.00 | 40.72 | C |
| ATOM | 1427 | C | THR | B2068 | 18.519 | −12.396 | 26.008 | 1.00 | 34.51 | C |
| ATOM | 1428 | O | THR | B2068 | 18.212 | −11.679 | 25.054 | 1.00 | 34.74 | O |
| ATOM | 1429 | N | GLY | B2069 | 17.715 | −12.598 | 27.051 | 1.00 | 34.71 | N |
| ATOM | 1430 | CA | GLY | B2069 | 16.403 | −11.970 | 27.132 | 1.00 | 33.79 | C |
| ATOM | 1431 | C | GLY | B2069 | 16.195 | −11.248 | 28.452 | 1.00 | 33.36 | C |
| ATOM | 1432 | O | GLY | B2069 | 16.823 | −11.590 | 29.455 | 1.00 | 32.35 | O |
| ATOM | 1433 | N | ILE | B2070 | 15.300 | −10.263 | 28.467 | 1.00 | 33.38 | N |
| ATOM | 1434 | CA | ILE | B2070 | 15.059 | −9.487 | 29.680 | 1.00 | 34.29 | C |
| ATOM | 1435 | CB | ILE | B2070 | 14.122 | −8.283 | 29.401 | 1.00 | 33.36 | C |
| ATOM | 1436 | CG2 | ILE | B2070 | 12.761 | −8.768 | 28.904 | 1.00 | 35.55 | C |
| ATOM | 1437 | CG1 | ILE | B2070 | 13.966 | −7.446 | 30.672 | 1.00 | 32.13 | C |
| ATOM | 1438 | CD1 | ILE | B2070 | 15.277 | −6.991 | 31.269 | 1.00 | 28.50 | C |
| ATOM | 1439 | C | ILE | B2070 | 14.514 | −10.295 | 30.856 | 1.00 | 35.39 | C |
| ATOM | 1440 | O | ILE | B2070 | 14.868 | −10.039 | 32.008 | 1.00 | 35.52 | O |
| ATOM | 1441 | N | SER | B2071 | 13.666 | −11.276 | 30.572 | 1.00 | 37.28 | N |
| ATOM | 1442 | CA | SER | B2071 | 13.104 | −12.104 | 31.630 | 1.00 | 39.77 | C |
| ATOM | 1443 | CB | SER | B2071 | 12.020 | −13.025 | 31.066 | 1.00 | 42.43 | C |
| ATOM | 1444 | OG | SER | B2071 | 11.433 | −13.795 | 32.102 | 1.00 | 46.76 | O |
| ATOM | 1445 | C | SER | B2071 | 14.189 | −12.947 | 32.299 | 1.00 | 40.07 | C |
| ATOM | 1446 | O | SER | B2071 | 14.024 | −13.402 | 33.428 | 1.00 | 39.81 | O |
| ATOM | 1447 | N | LYS | B2072 | 15.299 | −13.154 | 31.596 | 1.00 | 39.77 | N |
| ATOM | 1448 | CA | LYS | B2072 | 16.403 | −13.940 | 32.132 | 1.00 | 39.15 | C |
| ATOM | 1449 | CB | LYS | B2072 | 17.115 | −14.689 | 31.007 | 1.00 | 40.68 | C |
| ATOM | 1450 | CG | LYS | B2072 | 16.225 | −15.656 | 30.252 | 1.00 | 43.38 | C |
| ATOM | 1451 | CD | LYS | B2072 | 16.996 | −16.371 | 29.151 | 1.00 | 44.69 | C |
| ATOM | 1452 | CE | LYS | B2072 | 16.070 | −17.260 | 28.342 | 1.00 | 46.81 | C |
| ATOM | 1453 | NZ | LYS | B2072 | 15.337 | −18.225 | 29.214 | 1.00 | 47.85 | N |
| ATOM | 1454 | C | LYS | B2072 | 17.396 | −13.039 | 32.842 | 1.00 | 38.30 | C |
| ATOM | 1455 | O | LYS | B2072 | 17.952 | −13.401 | 33.879 | 1.00 | 38.50 | O |
| ATOM | 1456 | N | LEU | B2073 | 17.613 | −11.860 | 32.269 | 1.00 | 36.74 | N |
| ATOM | 1457 | CA | LEU | B2073 | 18.540 | −10.883 | 32.821 | 1.00 | 34.89 | C |
| ATOM | 1458 | CB | LEU | B2073 | 18.817 | −9.803 | 31.766 | 1.00 | 35.18 | C |
| ATOM | 1459 | CG | LEU | B2073 | 20.010 | −8.856 | 31.910 | 1.00 | 35.71 | C |
| ATOM | 1460 | CD1 | LEU | B2073 | 19.804 | −7.943 | 33.107 | 1.00 | 37.71 | C |
| ATOM | 1461 | CD2 | LEU | B2073 | 21.296 | −9.661 | 32.050 | 1.00 | 36.74 | C |
| ATOM | 1462 | C | LEU | B2073 | 17.961 | −10.259 | 34.098 | 1.00 | 34.21 | C |
| ATOM | 1463 | O | LEU | B2073 | 18.683 | −10.044 | 35.071 | 1.00 | 32.72 | O |
| ATOM | 1464 | N | GLY | B2074 | 16.658 | −9.982 | 34.090 | 1.00 | 33.08 | N |
| ATOM | 1465 | CA | GLY | B2074 | 16.014 | −9.383 | 35.247 | 1.00 | 32.12 | C |
| ATOM | 1466 | C | GLY | B2074 | 15.762 | −7.897 | 35.053 | 1.00 | 30.96 | C |
| ATOM | 1467 | O | GLY | B2074 | 16.702 | −7.127 | 34.871 | 1.00 | 31.73 | O |
| ATOM | 1468 | N | PHE | B2075 | 14.497 | −7.489 | 35.095 | 1.00 | 29.97 | N |
| ATOM | 1469 | CA | PHE | B2075 | 14.139 | −6.083 | 34.913 | 1.00 | 30.52 | C |
| ATOM | 1470 | CB | PHE | B2075 | 12.620 | −5.896 | 34.973 | 1.00 | 32.55 | C |
| ATOM | 1471 | CG | PHE | B2075 | 11.880 | −6.511 | 33.822 | 1.00 | 33.89 | C |
| ATOM | 1472 | CD1 | PHE | B2075 | 11.474 | −7.843 | 33.868 | 1.00 | 35.06 | C |
| ATOM | 1473 | CD2 | PHE | B2075 | 11.578 | −5.755 | 32.693 | 1.00 | 34.72 | C |
| ATOM | 1474 | CE1 | PHE | B2075 | 10.774 | −8.412 | 32.806 | 1.00 | 35.34 | C |
| ATOM | 1475 | CE2 | PHE | B2075 | 10.879 | −6.316 | 31.623 | 1.00 | 34.77 | C |
| ATOM | 1476 | CZ | PHE | B2075 | 10.477 | −7.646 | 31.681 | 1.00 | 34.64 | C |
| ATOM | 1477 | C | PHE | B2075 | 14.769 | −5.146 | 35.934 | 1.00 | 29.45 | C |
| ATOM | 1478 | O | PHE | B2075 | 15.159 | −4.025 | 35.599 | 1.00 | 30.37 | O |
| ATOM | 1479 | N | GLN | B2076 | 14.867 | −5.607 | 37.177 | 1.00 | 28.05 | N |
| ATOM | 1480 | CA | GLN | B2076 | 15.417 | −4.796 | 38.259 | 1.00 | 27.24 | C |
| ATOM | 1481 | CB | GLN | B2076 | 15.043 | −5.420 | 39.607 | 1.00 | 27.56 | C |
| ATOM | 1482 | CG | GLN | B2076 | 13.539 | −5.436 | 39.859 | 1.00 | 27.74 | C |
| ATOM | 1483 | CD | GLN | B2076 | 12.929 | −4.041 | 39.834 | 1.00 | 30.00 | C |
| ATOM | 1484 | OE1 | GLN | B2076 | 11.896 | −3.807 | 39.193 | 1.00 | 31.13 | O |
| ATOM | 1485 | NE2 | GLN | B2076 | 13.563 | −3.107 | 40.536 | 1.00 | 28.51 | N |
| ATOM | 1486 | C | GLN | B2076 | 16.920 | −4.538 | 38.198 | 1.00 | 26.61 | C |
| ATOM | 1487 | O | GLN | B2076 | 17.430 | −3.698 | 38.935 | 1.00 | 25.98 | O |
| ATOM | 1488 | N | ASP | B2077 | 17.621 | −5.251 | 37.320 | 1.00 | 26.64 | N |
| ATOM | 1489 | CA | ASP | B2077 | 19.062 | −5.084 | 37.160 | 1.00 | 27.59 | C |
| ATOM | 1490 | CB | ASP | B2077 | 19.723 | −6.426 | 36.847 | 1.00 | 32.09 | C |
| ATOM | 1491 | CG | ASP | B2077 | 19.749 | −7.352 | 38.041 | 1.00 | 35.66 | C |
| ATOM | 1492 | OD1 | ASP | B2077 | 18.670 | −7.841 | 38.440 | 1.00 | 39.93 | O |
| ATOM | 1493 | OD2 | ASP | B2077 | 20.850 | −7.584 | 38.586 | 1.00 | 40.87 | O |
| ATOM | 1494 | C | ASP | B2077 | 19.403 | −4.087 | 36.054 | 1.00 | 26.77 | C |
| ATOM | 1495 | O | ASP | B2077 | 20.571 | −3.825 | 35.779 | 1.00 | 25.66 | O |
| ATOM | 1496 | N | LEU | B2078 | 18.375 | −3.539 | 35.420 | 1.00 | 24.12 | N |
| ATOM | 1497 | CA | LEU | B2078 | 18.570 | −2.571 | 34.356 | 1.00 | 23.25 | C |
| ATOM | 1498 | CB | LEU | B2078 | 18.058 | −3.118 | 33.018 | 1.00 | 23.97 | C |
| ATOM | 1499 | CG | LEU | B2078 | 18.750 | −4.370 | 32.475 | 1.00 | 24.92 | C |
| ATOM | 1500 | CD1 | LEU | B2078 | 17.987 | −4.914 | 31.276 | 1.00 | 23.04 | C |
| ATOM | 1501 | CD2 | LEU | B2078 | 20.180 | −4.021 | 32.089 | 1.00 | 26.74 | C |
| ATOM | 1502 | C | LEU | B2078 | 17.795 | −1.321 | 34.702 | 1.00 | 23.29 | C |
| ATOM | 1503 | O | LEU | B2078 | 16.669 | −1.390 | 35.196 | 1.00 | 23.17 | O |
| ATOM | 1504 | N | GLU | B2079 | 18.397 | −0.173 | 34.446 | 1.00 | 21.42 | N |
| ATOM | 1505 | CA | GLU | B2079 | 17.724 | 1.075 | 34.716 | 1.00 | 21.45 | C |
| ATOM | 1506 | CB | GLU | B2079 | 18.192 | 1.666 | 36.036 | 1.00 | 22.77 | C |
| ATOM | 1507 | CG | GLU | B2079 | 17.346 | 2.835 | 36.477 | 1.00 | 24.03 | C |
| ATOM | 1508 | CD | GLU | B2079 | 17.726 | 3.340 | 37.848 | 1.00 | 25.73 | C |
| ATOM | 1509 | OE1 | GLU | B2079 | 18.797 | 3.972 | 37.976 | 1.00 | 26.42 | O |
| ATOM | 1510 | OE2 | GLU | B2079 | 16.950 | 3.099 | 38.798 | 1.00 | 27.63 | O |
| ATOM | 1511 | C | GLU | B2079 | 18.014 | 2.055 | 33.600 | 1.00 | 20.55 | C |
| ATOM | 1512 | O | GLU | B2079 | 19.167 | 2.241 | 33.203 | 1.00 | 20.96 | O |
| ATOM | 1513 | N | VAL | B2080 | 16.954 | 2.666 | 33.095 | 1.00 | 18.00 | N |
| ATOM | 1514 | CA | VAL | B2080 | 17.055 | 3.648 | 32.033 | 1.00 | 18.55 | C |
| ATOM | 1515 | CB | VAL | B2080 | 16.268 | 3.211 | 30.769 | 1.00 | 16.14 | C |
| ATOM | 1516 | CG1 | VAL | B2080 | 16.429 | 4.262 | 29.686 | 1.00 | 16.12 | C |
| ATOM | 1517 | CG2 | VAL | B2080 | 16.755 | 1.849 | 30.276 | 1.00 | 14.67 | C |
| ATOM | 1518 | C | VAL | B2080 | 16.442 | 4.948 | 32.549 | 1.00 | 19.85 | C |
| ATOM | 1519 | O | VAL | B2080 | 15.234 | 5.019 | 32.805 | 1.00 | 19.59 | O |
| ATOM | 1520 | N | LEU | B2081 | 17.279 | 5.966 | 32.708 | 1.00 | 20.39 | N |
| ATOM | 1521 | CA | LEU | B2081 | 16.825 | 7.271 | 33.176 | 1.00 | 21.24 | C |
| ATOM | 1522 | CB | LEU | B2081 | 17.644 | 7.716 | 34.394 | 1.00 | 21.31 | C |
| ATOM | 1523 | CG | LEU | B2081 | 17.683 | 6.758 | 35.593 | 1.00 | 22.71 | C |
| ATOM | 1524 | CD1 | LEU | B2081 | 18.641 | 7.299 | 36.661 | 1.00 | 23.10 | C |
| ATOM | 1525 | CD2 | LEU | B2081 | 16.280 | 6.594 | 36.165 | 1.00 | 22.25 | C |
| ATOM | 1526 | C | LEU | B2081 | 17.027 | 8.259 | 32.040 | 1.00 | 22.16 | C |
| ATOM | 1527 | O | LEU | B2081 | 17.421 | 7.882 | 30.934 | 1.00 | 22.05 | O |
| ATOM | 1528 | N | ASN | B2082 | 16.755 | 9.526 | 32.309 | 1.00 | 22.54 | N |
| ATOM | 1529 | CA | ASN | B2082 | 16.933 | 10.561 | 31.303 | 1.00 | 24.30 | C |
| ATOM | 1530 | CB | ASN | B2082 | 15.598 | 11.247 | 31.022 | 1.00 | 25.64 | C |
| ATOM | 1531 | CG | ASN | B2082 | 14.657 | 10.371 | 30.207 | 1.00 | 27.22 | C |
| ATOM | 1532 | OD1 | ASN | B2082 | 14.779 | 10.280 | 28.982 | 1.00 | 28.88 | O |
| ATOM | 1533 | ND2 | ASN | B2082 | 13.729 | 9.710 | 30.883 | 1.00 | 24.27 | N |
| ATOM | 1534 | C | ASN | B2082 | 17.945 | 11.555 | 31.840 | 1.00 | 25.00 | C |
| ATOM | 1535 | O | ASN | B2082 | 17.860 | 11.958 | 33.001 | 1.00 | 23.46 | O |
| ATOM | 1536 | N | ASN | B2083 | 18.913 | 11.939 | 31.010 | 1.00 | 25.22 | N |
| ATOM | 1537 | CA | ASN | B2083 | 19.920 | 12.890 | 31.454 | 1.00 | 25.69 | C |
| ATOM | 1538 | CB | ASN | B2083 | 21.215 | 12.744 | 30.641 | 1.00 | 25.99 | C |
| ATOM | 1539 | CG | ASN | B2083 | 21.013 | 12.961 | 29.153 | 1.00 | 27.46 | C |
| ATOM | 1540 | OD1 | ASN | B2083 | 20.173 | 13.758 | 28.729 | 1.00 | 25.62 | O |
| ATOM | 1541 | ND2 | ASN | B2083 | 21.811 | 12.265 | 28.348 | 1.00 | 27.23 | N |
| ATOM | 1542 | C | ASN | B2083 | 19.410 | 14.330 | 31.397 | 1.00 | 26.79 | C |
| ATOM | 1543 | O | ASN | B2083 | 18.216 | 14.571 | 31.215 | 1.00 | 25.77 | O |
| ATOM | 1544 | N | GLU | B2084 | 20.322 | 15.281 | 31.559 | 1.00 | 29.60 | N |
| ATOM | 1545 | CA | GLU | B2084 | 19.982 | 16.701 | 31.563 | 1.00 | 31.78 | C |
| ATOM | 1546 | CB | GLU | B2084 | 21.238 | 17.539 | 31.827 | 1.00 | 33.94 | C |
| ATOM | 1547 | CG | GLU | B2084 | 22.148 | 16.960 | 32.896 | 1.00 | 39.23 | C |
| ATOM | 1548 | CD | GLU | B2084 | 22.774 | 15.640 | 32.470 | 1.00 | 41.35 | C |
| ATOM | 1549 | OE1 | GLU | B2084 | 23.642 | 15.652 | 31.567 | 1.00 | 42.45 | O |
| ATOM | 1550 | OE2 | GLU | B2084 | 22.390 | −14.588 | 33.030 | 1.00 | 42.21 | O |
| ATOM | 1551 | C | GLU | B2084 | 19.351 | 17.153 | 30.253 | 1.00 | 30.81 | C |
| ATOM | 1552 | O | GLU | B2084 | 18.472 | 18.013 | 30.242 | 1.00 | 29.26 | O |
| ATOM | 1553 | N | ARG | B2085 | 19.808 | 16.573 | 29.148 | 1.00 | 30.86 | N |
| ATOM | 1554 | CA | ARG | B2085 | 19.291 | 16.940 | 27.838 | 1.00 | 31.05 | C |
| ATOM | 1555 | CB | ARG | B2085 | 20.364 | 16.716 | 26.775 | 1.00 | 33.27 | C |
| ATOM | 1556 | CG | ARG | B2085 | 21.598 | 17.554 | 27.005 | 1.00 | 35.81 | C |
| ATOM | 1557 | CD | ARG | B2085 | 22.636 | 17.335 | 25.928 | 1.00 | 38.24 | C |
| ATOM | 1558 | NE | ARG | B2085 | 23.735 | 18.286 | 26.060 | 1.00 | 41.03 | N |
| ATOM | 1559 | CZ | ARG | B2085 | 24.544 | 18.349 | 27.110 | 1.00 | 42.41 | C |
| ATOM | 1560 | NH1 | ARG | B2085 | 24.381 | 17.515 | 28.128 | 1.00 | 43.26 | N |
| ATOM | 1561 | NH2 | ARG | B2085 | 25.523 | 19.239 | 27.138 | 1.00 | 44.30 | N |
| ATOM | 1562 | C | ARG | B2085 | 18.024 | 16.190 | 27.465 | 1.00 | 30.14 | C |
| ATOM | 1563 | O | ARG | B2085 | 17.450 | 16.432 | 26.408 | 1.00 | 31.11 | O |
| ATOM | 1564 | N | GLY | B2086 | 17.593 | 15.273 | 28.326 | 1.00 | 29.20 | N |
| ATOM | 1565 | CA | GLY | B2086 | 16.371 | 14.530 | 28.057 | 1.00 | 27.93 | C |
| ATOM | 1566 | C | GLY | B2086 | 16.531 | 13.222 | 27.304 | 1.00 | 26.69 | C |
| ATOM | 1567 | O | GLY | B2086 | 15.541 | 12.581 | 26.952 | 1.00 | 27.22 | O |
| ATOM | 1568 | N | ALA | B2087 | 17.770 | 12.818 | 27.056 | 1.00 | 24.98 | N |
| ATOM | 1569 | CA | ALA | B2087 | 18.033 | 11.582 | 26.340 | 1.00 | 23.11 | C |
| ATOM | 1570 | CB | ALA | B2087 | 19.358 | 11.692 | 25.589 | 1.00 | 24.73 | C |
| ATOM | 1571 | C | ALA | B2087 | 18.065 | 10.369 | 27.269 | 1.00 | 22.69 | C |
| ATOM | 1572 | O | ALA | B2087 | 18.611 | 10.431 | 28.370 | 1.00 | 22.81 | O |
| ATOM | 1573 | N | PRO | B2088 | 17.481 | 9.242 | 26.833 | 1.00 | 22.85 | N |
| ATOM | 1574 | CD | PRO | B2088 | 16.722 | 9.014 | 25.588 | 1.00 | 23.63 | C |
| ATOM | 1575 | CA | PRO | B2088 | 17.485 | 8.039 | 27.674 | 1.00 | 21.17 | C |
| ATOM | 1576 | CB | PRO | B2088 | 16.471 | 7.134 | 26.982 | 1.00 | 22.95 | C |
| ATOM | 1577 | CG | PRO | B2088 | 16.650 | 7.489 | 25.528 | 1.00 | 24.16 | C |
| ATOM | 1578 | C | PRO | B2088 | 18.879 | 7.416 | 27.716 | 1.00 | 20.46 | C |
| ATOM | 1579 | O | PRO | B2088 | 19.584 | 7.407 | 26.712 | 1.00 | 18.12 | O |
| ATOM | 1580 | N | TYR | B2089 | 19.280 | 6.906 | 28.878 | 1.00 | 19.76 | N |
| ATOM | 1581 | CA | TYR | B2089 | 20.593 | 6.275 | 29.019 | 1.00 | 19.97 | C |
| ATOM | 1582 | CB | TYR | B2089 | 21.660 | 7.337 | 29.324 | 1.00 | 20.30 | C |
| ATOM | 1583 | CG | TYR | B2089 | 21.605 | 7.850 | 30.743 | 1.00 | 21.25 | C |
| ATOM | 1584 | CD1 | TYR | B2089 | 22.471 | 7.347 | 31.719 | 1.00 | 23.06 | C |
| ATOM | 1585 | CE1 | TYR | B2089 | 22.386 | 7.765 | 33.046 | 1.00 | 22.62 | C |
| ATOM | 1586 | CD2 | TYR | B2089 | 20.651 | 8.793 | 31.128 | 1.00 | 21.67 | C |
| ATOM | 1587 | CE2 | TYR | B2089 | 20.557 | 9.218 | 32.454 | 1.00 | 23.51 | C |
| ATOM | 1588 | CZ | TYR | B2089 | 21.429 | 8.693 | 33.404 | 1.00 | 22.23 | C |
| ATOM | 1589 | OH | TYR | B2089 | 21.327 | 9.078 | 34.715 | 1.00 | 23.91 | O |
| ATOM | 1590 | C | TYR | B2089 | 20.547 | 5.246 | 30.148 | 1.00 | 18.84 | C |
| ATOM | 1591 | O | TYR | B2089 | 19.738 | 5.356 | 31.066 | 1.00 | 16.92 | O |
| ATOM | 1592 | N | PHE | B2090 | 21.408 | 4.237 | 30.076 | 1.00 | 19.96 | N |
| ATOM | 1593 | CA | PHE | B2090 | 21.432 | 3.218 | 31.116 | 1.00 | 20.18 | C |
| ATOM | 1594 | CB | PHE | B2090 | 22.015 | 1.906 | 30.588 | 1.00 | 18.86 | C |
| ATOM | 1595 | CG | PHE | B2090 | 21.031 | 1.078 | 29.813 | 1.00 | 17.77 | C |
| ATOM | 1596 | CD1 | PHE | B2090 | 20.843 | 1.282 | 28.452 | 1.00 | 18.34 | C |
| ATOM | 1597 | CD2 | PHE | B2090 | 20.276 | 0.102 | 30.455 | 1.00 | 17.66 | C |
| ATOM | 1598 | CE1 | PHE | B2090 | 19.915 | 0.523 | 27.737 | 1.00 | 16.58 | C |
| ATOM | 1599 | CE2 | PHE | B2090 | 19.344 | −0.666 | 29.751 | 1.00 | 16.83 | C |
| ATOM | 1600 | CZ | PHE | B2090 | 19.166 | −0.453 | 28.393 | 1.00 | 18.42 | C |
| ATOM | 1601 | C | PHE | B2090 | 22.249 | 3.683 | 32.307 | 1.00 | 21.96 | C |
| ATOM | 1602 | O | PHE | B2090 | 23.464 | 3.837 | 32.215 | 1.00 | 21.17 | O |
| ATOM | 1603 | N | SER | B2091 | 21.569 | 3.916 | 33.423 | 1.00 | 21.79 | N |
| ATOM | 1604 | CA | SER | B2091 | 22.233 | 4.346 | 34.642 | 1.00 | 23.26 | C |
| ATOM | 1605 | CB | SER | B2091 | 21.269 | 5.171 | 35.498 | 1.00 | 24.51 | C |
| ATOM | 1606 | OG | SER | B2091 | 20.046 | 4.473 | 35.689 | 1.00 | 27.15 | O |
| ATOM | 1607 | C | SER | B2091 | 22.676 | −3.101 | 35.406 | 1.00 | 24.05 | C |
| ATOM | 1608 | O | SER | B2091 | 23.572 | 3.158 | 36.241 | 1.00 | 22.27 | O |
| ATOM | 1609 | N | GLN | B2092 | 22.039 | 1.974 | 35.100 | 1.00 | 24.23 | N |
| ATOM | 1610 | CA | GLN | B2092 | 22.343 | 0.702 | 35.750 | 1.00 | 26.68 | C |
| ATOM | 1611 | CB | GLN | B2092 | 21.324 | 0.420 | 36.858 | 1.00 | 30.19 | C |
| ATOM | 1612 | CG | GLN | B2092 | 21.738 | 0.888 | 38.236 | 1.00 | 36.96 | C |
| ATOM | 1613 | CD | GLN | B2092 | 22.791 | −0.013 | 38.846 | 1.00 | 41.69 | C |
| ATOM | 1614 | OE1 | GLN | B2092 | 23.923 | −0.086 | 38.360 | 1.00 | 44.08 | O |
| ATOM | 1615 | NE2 | GLN | B2092 | 22.419 | −0.719 | 39.914 | 1.00 | 43.86 | N |
| ATOM | 1616 | C | GLN | B2092 | 22.318 | −0.450 | 34.755 | 1.00 | 25.68 | C |
| ATOM | 1617 | O | GLN | B2092 | 21.350 | −0.628 | 34.015 | 1.00 | 25.64 | O |
| ATOM | 1618 | N | ALA | B2093 | 23.378 | −1.243 | 34.750 | 1.00 | 24.06 | N |
| ATOM | 1619 | CA | ALA | B2093 | 23.451 | −2.379 | 33.845 | 1.00 | 24.55 | C |
| ATOM | 1620 | CB | ALA | B2093 | 23.544 | −1.896 | 32.401 | 1.00 | 23.95 | C |
| ATOM | 1621 | C | ALA | B2093 | 24.654 | −3.240 | 34.182 | 1.00 | 24.42 | C |
| ATOM | 1622 | O | ALA | B2093 | 25.669 | −2.737 | 34.662 | 1.00 | 23.73 | O |
| ATOM | 1623 | N | PRO | B2094 | 24.542 | −4.560 | 33.958 | 1.00 | 24.28 | N |
| ATOM | 1624 | CD | PRO | B2094 | 23.286 | −5.266 | 33.634 | 1.00 | 24.45 | C |
| ATOM | 1625 | CA | PRO | B2094 | 25.625 | −5.509 | 34.226 | 1.00 | 24.13 | C |
| ATOM | 1626 | CB | PRO | B2094 | 24.882 | −6.827 | 34.404 | 1.00 | 24.53 | C |
| ATOM | 1627 | CG | PRO | B2094 | 23.752 | −6.693 | 33.412 | 1.00 | 25.43 | C |
| ATOM | 1628 | C | PRO | B2094 | 26.575 | −5.532 | 33.033 | 1.00 | 25.18 | C |
| ATOM | 1629 | O | PRO | B2094 | 26.687 | −6.538 | 32.330 | 1.00 | 27.39 | O |
| ATOM | 1630 | N | PHE | B2095 | 27.237 | −4.406 | 32.795 | 1.00 | 25.59 | N |
| ATOM | 1631 | CA | PHE | B2095 | 28.166 | −4.276 | 31.674 | 1.00 | 26.28 | C |
| ATOM | 1632 | CB | PHE | B2095 | 27.394 | −3.937 | 30.392 | 1.00 | 26.01 | C |
| ATOM | 1633 | CG | PHE | B2095 | 28.264 | −3.808 | 29.176 | 1.00 | 25.16 | C |
| ATOM | 1634 | CD1 | PHE | B2095 | 28.289 | −2.622 | 28.448 | 1.00 | 23.33 | C |
| ATOM | 1635 | CD2 | PHE | B2095 | 29.070 | −4.864 | 28.766 | 1.00 | 24.13 | C |
| ATOM | 1636 | CE1 | PHE | B2095 | 29.106 | −2.488 | 27.333 | 1.00 | 23.09 | C |
| ATOM | 1637 | CE2 | PHE | B2095 | 29.890 | −4.739 | 27.652 | 1.00 | 24.48 | C |
| ATOM | 1638 | CZ | PHE | B2095 | 29.906 | −3.542 | 26.933 | 1.00 | 21.85 | C |
| ATOM | 1639 | C | PHE | B2095 | 29.157 | −3.168 | 32.000 | 1.00 | 25.62 | C |
| ATOM | 1640 | O | PHE | B2095 | 28.768 | −2.080 | 32.413 | 1.00 | 26.70 | O |
| ATOM | 1641 | N | SER | B2096 | 30.438 | −3.441 | .31.804 | 1.00 | 26.81 | N |
| ATOM | 1642 | CA | SER | B2096 | 31.472 | −2.466 | 32.122 | 1.00 | 27.94 | C |
| ATOM | 1643 | CB | SER | B2096 | 32.750 | −3.203 | 32.531 | 1.00 | 27.66 | C |
| ATOM | 1644 | OG | SER | B2096 | 33.144 | −4.111 | 31.518 | 1.00 | 29.62 | O |
| ATOM | 1645 | C | SER | B2096 | 31.787 | −1.469 | 31.007 | 1.00 | 27.71 | C |
| ATOM | 1646 | O | SER | B2096 | 32.430 | −0.452 | 31.249 | 1.00 | 27.75 | O |
| ATOM | 1647 | N | GLY | B2097 | 31.325 | −1.746 | 29.792 | 1.00 | 26.55 | N |
| ATOM | 1648 | CA | GLY | B2097 | 31.611 | −0.847 | 28.690 | 1.00 | 23.99 | C |
| ATOM | 1649 | C | GLY | B2097 | 30.611 | 0.274 | 28.499 | 1.00 | 23.68 | C |
| ATOM | 1650 | O | GLY | B2097 | 29.869 | 0.639 | 29.413 | 1.00 | 22.73 | O |
| ATOM | 1651 | N | LYS | B2098 | 30.606 | 0.838 | 27.299 | 1.00 | 23.32 | N |
| ATOM | 1652 | CA | LYS | B2098 | 29.682 | 1.912 | 26.975 | 1.00 | 23.52 | C |
| ATOM | 1653 | CB | LYS | B2098 | 30.323 | 2.896 | 25.995 | 1.00 | 25.66 | C |
| ATOM | 1654 | CG | LYS | B2098 | 31.546 | 3.599 | 26.562 | 1.00 | 29.30 | C |
| ATOM | 1655 | CD | LYS | B2098 | 32.162 | 4.532 | 25.545 | 1.00 | 31.81 | C |
| ATOM | 1656 | CE | LYS | B2098 | 33.364 | 5.247 | 26.128 | 1.00 | 35.72 | C |
| ATOM | 1657 | NZ | LYS | B2098 | 33.970 | 6.194 | 25.154 | 1.00 | 38.45 | N |
| ATOM | 1658 | C | LYS | B2098 | 28.438 | 1.302 | 26.356 | 1.00 | 21.94 | C |
| ATOM | 1659 | O | LYS | B2098 | 28.518 | 0.350 | 25.570 | 1.00 | 20.64 | O |
| ATOM | 1660 | N | ILE | B2099 | 27.294 | 1.862 | 26.721 | 1.00 | 20.44 | N |
| ATOM | 1661 | CA | ILE | B2099 | 26.008 | 1.400 | 26.236 | 1.00 | 19.38 | C |
| ATOM | 1662 | CB | ILE | B2099 | 25.109 | 1.012 | 27.421 | 1.00 | 19.92 | C |
| ATOM | 1663 | CG2 | ILE | B2099 | 23.804 | 0.423 | 26.919 | 1.00 | 20.26 | C |
| ATOM | 1664 | CG1 | ILE | B2099 | 25.839 | −0.008 | 28.302 | 1.00 | 19.45 | C |
| ATOM | 1665 | CD1 | ILE | B2099 | 25.193 | −0.230 | 29.663 | 1.00 | 21.98 | C |
| ATOM | 1666 | C | ILE | B2099 | 25.352 | 2.529 | 25.444 | 1.00 | 19.88 | C |
| ATOM | 1667 | O | ILE | B2099 | 24.950 | 3.546 | 26.011 | 1.00 | 19.12 | O |
| ATOM | 1668 | N | TRP | B2100 | 25.267 | 2.352 | 24.129 | 1.00 | 19.02 | N |
| ATOM | 1669 | CA | TRP | B2100 | 24.662 | 3.351 | 23.265 | 1.00 | 17.64 | C |
| ATOM | 1670 | CB | TRP | B2100 | 25.430 | 3.431 | 21.951 | 1.00 | 19.81 | C |
| ATOM | 1671 | CG | TRP | B2100 | 26.840 | 3.861 | 22.165 | 1.00 | 20.97 | C |
| ATOM | 1672 | CD2 | TRP | B2100 | 27.295 | 5.202 | 22.383 | 1.00 | 21.20 | C |
| ATOM | 1673 | CE2 | TRP | B2100 | 28.680 | 5.137 | 22.640 | 1.00 | 20.87 | C |
| ATOM | 1674 | CE3 | TRP | B2100 | 26.661 | 6.454 | 22.393 | 1.00 | 22.40 | C |
| ATOM | 1675 | CD1 | TRP | B2100 | 27.938 | 3.059 | 22.289 | 1.00 | 22.80 | C |
| ATOM | 1676 | NE1 | TRP | B2100 | 29.050 | 3.818 | 22.576 | 1.00 | 21.53 | N |
| ATOM | 1677 | CZ2 | TRP | B2100 | 29.448 | 6.279 | 22.901 | 1.00 | 21.01 | C |
| ATOM | 1678 | CZ3 | TRP | B2100 | 27.425 | 7.591 | 22.654 | 1.00 | 19.76 | C |
| ATOM | 1679 | CH2 | TRP | B2100 | 28.803 | 7.492 | 22.905 | 1.00 | 20.24 | C |
| ATOM | 1680 | C | TRP | B2100 | 23.207 | 2.987 | 23.023 | 1.00 | 17.98 | C |
| ATOM | 1681 | O | TRP | B2100 | 22.889 | 2.033 | 22.308 | 1.00 | 19.71 | O |
| ATOM | 1682 | N | LEU | B2101 | 22.323 | 3.761 | 23.632 | 1.00 | 16.57 | N |
| ATOM | 1683 | CA | LEU | B2101 | 20.897 | 3.527 | 23.524 | 1.00 | 15.94 | C |
| ATOM | 1684 | CB | LEU | B2101 | 20.292 | 3.364 | 24.925 | 1.00 | 14.34 | C |
| ATOM | 1685 | CG | LEU | B2101 | 18.756 | 3.387 | 25.013 | 1.00 | 15.83 | C |
| ATOM | 1686 | CD1 | LEU | B2101 | 18.195 | 2.073 | 24.455 | 1.00 | 11.99 | C |
| ATOM | 1687 | CD2 | LEU | B2101 | 18.324 | 3.555 | 26.467 | 1.00 | 14.03 | C |
| ATOM | 1688 | C | LEU | B2101 | 20.142 | 4.632 | 22.810 | 1.00 | 16.46 | C |
| ATOM | 1689 | O | LEU | B2101 | 20.464 | 5.807 | 22.937 | 1.00 | 14.73 | O |
| ATOM | 1690 | N | SER | B2102 | 19.133 | 4.232 | 22.047 | 1.00 | 17.13 | N |
| ATOM | 1691 | CA | SER | B2102 | 18.260 | 5.182 | 21.380 | 1.00 | 16.87 | C |
| ATOM | 1692 | CB | SER | B2102 | 18.683 | 5.449 | 19.945 | 1.00 | 17.85 | C |
| ATOM | 1693 | OG | SER | B2102 | 17.856 | 6.473 | 19.405 | 1.00 | 18.12 | O |
| ATOM | 1694 | C | SER | B2102 | 16.866 | 4.577 | 21.393 | 1.00 | 15.72 | C |
| ATOM | 1695 | O | SER | B2102 | 16.706 | 3.368 | 21.230 | 1.00 | 14.46 | O |
| ATOM | 1696 | N | ILE | B2103 | 15.863 | 5.414 | 21.613 | 1.00 | 15.13 | N |
| ATOM | 1697 | CA | ILE | B2103 | 14.478 | 4.948 | 21.646 | 1.00 | 17.16 | C |
| ATOM | 1698 | CB | ILE | B2103 | 13.897 | 5.031 | 23.070 | 1.00 | 17.86 | C |
| ATOM | 1699 | CG2 | ILE | B2103 | 12.439 | 4.560 | 23.063 | 1.00 | 17.77 | C |
| ATOM | 1700 | CG1 | ILE | B2103 | 14.765 | 4.215 | 24.036 | 1.00 | 18.09 | C |
| ATOM | 1701 | CD1 | ILE | B2103 | 14.406 | 4.396 | 25.497 | 1.00 | 17.05 | C |
| ATOM | 1702 | C | ILE | B2103 | 13.645 | 5.852 | 20.749 | 1.00 | 17.44 | C |
| ATOM | 1703 | O | ILE | B2103 | 13.886 | 7.055 | 20.684 | 1.00 | 17.70 | O |
| ATOM | 1704 | N | SER | B2104 | 12.675 | 5.275 | 20.053 | 1.00 | 17.76 | N |
| ATOM | 1705 | CA | SER | B2104 | 11.803 | 6.065 | 19.187 | 1.00 | 19.81 | C |
| ATOM | 1706 | CB | SER | B2104 | 12.355 | 6.117 | 17.759 | 1.00 | 19.27 | C |
| ATOM | 1707 | OG | SER | B2104 | 11.570 | 6.964 | 16.930 | 1.00 | 20.56 | O |
| ATOM | 1708 | C | SER | B2104 | 10.426 | 5.432 | 19.189 | 1.00 | 20.64 | C |
| ATOM | 1709 | O | SER | B2104 | 10.290 | 4.213 | 19.338 | 1.00 | 22.59 | O |
| ATOM | 1710 | N | HIS | B2105 | 9.396 | 6.246 | 19.020 | 1.00 | 20.13 | N |
| ATOM | 1711 | CA | HIS | B2105 | 8.055 | 5.698 | 19.028 | 1.00 | 22.93 | C |
| ATOM | 1712 | CB | HIS | B2105 | 7.456 | 5.812 | 20.432 | 1.00 | 23.19 | C |
| ATOM | 1713 | CG | HIS | B2105 | 7.277 | 7.227 | 20.890 | 1.00 | 24.84 | C |
| ATOM | 1714 | CD2 | HIS | B2105 | 6.221 | 8.069 | 20.785 | 1.00 | 26.28 | C |
| ATOM | 1715 | ND1 | HIS | B2105 | 8.284 | 7.950 | 21.490 | 1.00 | 26.53 | N |
| ATOM | 1716 | CE1 | HIS | B2105 | 7.858 | 9.176 | 21.737 | 1.00 | 25.54 | C |
| ATOM | 1717 | NE2 | HIS | B2105 | 6.609 | 9.275 | 21.318 | 1.00 | 26.28 | N |
| ATOM | 1718 | C | HIS | B2105 | 7.116 | 6.375 | 18.050 | 1.00 | 21.93 | C |
| ATOM | 1719 | O | HIS | B2105 | 7.395 | 7.460 | 17.554 | 1.00 | 21.72 | O |
| ATOM | 1720 | N | THR | B2106 | 6.016 | 5.687 | 17.769 | 1.00 | 23.48 | N |
| ATOM | 1721 | CA | THR | B2106 | 4.941 | 6.183 | 16.919 | 1.00 | 25.16 | C |
| ATOM | 1722 | CB | THR | B2106 | 4.605 | 5.242 | 15.755 | 1.00 | 25.21 | C |
| ATOM | 1723 | OG1 | THR | B2106 | 4.222 | 3.965 | 16.278 | 1.00 | 25.03 | O |
| ATOM | 1724 | CG2 | THR | B2106 | 5.791 | 5.089 | 14.823 | 1.00 | 25.52 | C |
| ATOM | 1725 | C | THR | B2106 | 3.775 | 6.121 | 17.894 | 1.00 | 25.80 | C |
| ATOM | 1726 | O | THR | B2106 | 3.978 | 5.862 | 19.081 | 1.00 | 24.98 | O |
| ATOM | 1727 | N | ASP | B2107 | 2.559 | 6.329 | 17.406 | 1.00 | 25.90 | N |
| ATOM | 1728 | CA | ASP | B2107 | 1.397 | 6.284 | 18.285 | 1.00 | 27.49 | C |
| ATOM | 1729 | CB | ASP | B2107 | 0.169 | 6.862 | 17.574 | 0.05 | 26.86 | C |
| ATOM | 1730 | CG | ASP | B2107 | 0.127 | 8.380 | 17.621 | 0.05 | 26.92 | C |
| ATOM | 1731 | OD1 | ASP | B2107 | 1.090 | 9.023 | 17.157 | 0.05 | 27.01 | O |
| ATOM | 1732 | OD2 | ASP | B2107 | −0.874 | 8.930 | 18.125 | 0.05 | 26.97 | O |
| ATOM | 1733 | C | ASP | B2107 | 1.085 | 4.877 | 18.788 | 1.00 | 27.36 | C |
| ATOM | 1734 | O | ASP | B2107 | 0.493 | 4.717 | 19.854 | 1.00 | 29.78 | O |
| ATOM | 1735 | N | GLN | B2108 | 1.482 | 3.858 | 18.032 | 1.00 | 25.07 | N |
| ATOM | 1736 | CA | GLN | B2108 | 1.207 | 2.483 | 18.433 | 1.00 | 25.09 | C |
| ATOM | 1737 | CB | GLN | B2108 | 0.523 | 1.736 | 17.293 | 1.00 | 26.05 | C |
| ATOM | 1738 | CG | GLN | B2108 | −0.728 | 2.395 | 16.780 | 1.00 | 29.37 | C |
| ATOM | 1739 | CD | GLN | B2108 | −1.145 | 1.827 | 15.445 | 1.00 | 31.53 | C |
| ATOM | 1740 | OE1 | GLN | B2108 | −0.362 | 1.822 | 14.492 | 1.00 | 31.44 | O |
| ATOM | 1741 | NE2 | GLN | B2108 | −2.379 | 1.345 | 15.363 | 1.00 | 31.86 | N |
| ATOM | 1742 | C | GLN | B2108 | 2.412 | 1.658 | 18.876 | 1.00 | 24.58 | C |
| ATOM | 1743 | O | GLN | B2108 | 2.251 | 0.710 | 19.650 | 1.00 | 22.64 | O |
| ATOM | 1744 | N | PHE | B2109 | 3.606 | 2.000 | 18.386 | 1.00 | 23.20 | N |
| ATOM | 1745 | CA | PHE | B2109 | 4.800 | 1.229 | 18.725 | 1.00 | 22.81 | C |
| ATOM | 1746 | CB | PHE | B2109 | 5.309 | 0.456 | 17.501 | 1.00 | 24.68 | C |
| ATOM | 1747 | CG | PHE | B2109 | 4.236 | −0.250 | 16.721 | 1.00 | 26.08 | C |
| ATOM | 1748 | CD1 | PHE | B2109 | 3.701 | 0.329 | 15.576 | 1.00 | 26.47 | C |
| ATOM | 1749 | CD2 | PHE | B2109 | 3.783 | −1.505 | 17.110 | 1.00 | 27.02 | C |
| ATOM | 1750 | CE1 | PHE | B2109 | 2.729 | −0.336 | 14.825 | 1.00 | 28.36 | C |
| ATOM | 1751 | CE2 | PHE | B2109 | 2.810 | −2.177 | 16.366 | 1.00 | 27.54 | C |
| ATOM | 1752 | CZ | PHE | B2109 | 2.287 | −1.592 | 15.224 | 1.00 | 28.20 | C |
| ATOM | 1753 | C | PHE | B2109 | 5.980 | 2.037 | 19.260 | 1.00 | 21.56 | C |
| ATOM | 1754 | O | PHE | B2109 | 6.096 | 3.233 | 19.027 | 1.00 | 22.22 | O |
| ATOM | 1755 | N | VAL | B2110 | 6.872 | 1.358 | 19.969 | 1.00 | 21.04 | N |
| ATOM | 1756 | CA | VAL | B2110 | 8.073 | 2.005 | 20.485 | 1.00 | 20.21 | C |
| ATOM | 1757 | CB | VAL | B2110 | 7.982 | 2.279 | 22.006 | 1.00 | 19.74 | C |
| ATOM | 1758 | CG1 | VAL | B2110 | 7.649 | 1.001 | 22.751 | 1.00 | 21.12 | C |
| ATOM | 1759 | CG2 | VAL | B2110 | 9.307 | 2.861 | 22.504 | 1.00 | 21.25 | C |
| ATOM | 1760 | C | VAL | B2110 | 9.230 | 1.063 | 20.197 | 1.00 | 20.24 | C |
| ATOM | 1761 | O | VAL | B2110 | 9.160 | −0.128 | 20.523 | 1.00 | 21.27 | O |
| ATOM | 1762 | N | THR | B2111 | 10.276 | 1.580 | 19.557 | 1.00 | 19.29 | N |
| ATOM | 1763 | CA | THR | B2111 | 11.440 | 0.763 | 19.235 | 1.00 | 20.44 | C |
| ATOM | 1764 | CB | THR | B2111 | 11.759 | 0.788 | 17.725 | 1.00 | 22.75 | C |
| ATOM | 1765 | OG1 | THR | B2111 | 12.116 | 2.120 | 17.337 | 1.00 | 27.12 | O |
| ATOM | 1766 | CG2 | THR | B2111 | 10.550 | 0.353 | 16.911 | 1.00 | 26.61 | C |
| ATOM | 1767 | C | THR | B2111 | 12.668 | 1.270 | 19.982 | 1.00 | 19.21 | C |
| ATOM | 1768 | O | THR | B2111 | 12.780 | 2.455 | 20.278 | 1.00 | 18.06 | O |
| ATOM | 1769 | N | ALA | B2112 | 13.581 | 0.353 | 20.280 | 1.00 | 18.16 | N |
| ATOM | 1770 | CA | ALA | B2112 | 14.810 | 0.675 | 20.988 | 1.00 | 17.59 | C |
| ATOM | 1771 | CB | ALA | B2112 | 14.715 | 0.231 | 22.442 | 1.00 | 14.54 | C |
| ATOM | 1772 | C | ALA | B2112 | 15.951 | −0.052 | 20.292 | 1.00 | 17.15 | C |
| ATOM | 1773 | O | ALA | B2112 | 15.776 | −1.163 | 19.788 | 1.00 | 15.26 | O |
| ATOM | 1774 | N | SER | B2113 | 17.111 | 0.588 | 20.258 | 1.00 | 16.75 | N |
| ATOM | 1775 | CA | SER | B2113 | 18.297 | 0.015 | 19.631 | 1.00 | 17.12 | C |
| ATOM | 1776 | CB | SER | B2113 | 18.574 | 0.705 | 18.287 | 1.00 | 17.55 | C |
| ATOM | 1777 | OG | SER | B2113 | 19.674 | 0.112 | 17.603 | 1.00 | 19.24 | O |
| ATOM | 1778 | C | SER | B2113 | 19.470 | 0.227 | 20.588 | 1.00 | 16.82 | C |
| ATOM | 1779 | O | SER | B2113 | 19.677 | 1.327 | 21.092 | 1.00 | 17.25 | O |
| ATOM | 1780 | N | VAL | B2114 | 20.220 | −0.837 | 20.840 | 1.00 | 16.23 | N |
| ATOM | 1781 | CA | VAL | B2114 | 21.364 | −0.778 | 21.736 | 1.00 | 15.71 | C |
| ATOM | 1782 | CB | VAL | B2114 | 21.129 | −1.591 | 23.017 | 1.00 | 13.94 | C |
| ATOM | 1783 | CG1 | VAL | B2114 | 22.451 | −1.731 | 23.784 | 1.00 | 14.11 | C |
| ATOM | 1784 | CG2 | VAL | B2114 | 20.070 | −0.923 | 23.877 | 1.00 | 10.51 | C |
| ATOM | 1785 | C | VAL | B2114 | 22.617 | −1.345 | 21.106 | 1.00 | 16.39 | C |
| ATOM | 1786 | O | VAL | B2114 | 22.578 | −2.412 | 20.492 | 1.00 | 16.76 | O |
| ATOM | 1787 | N | ILE | B2115 | 23.724 | −0.625 | 21.259 | 1.00 | 14.68 | N |
| ATOM | 1788 | CA | ILE | B2115 | 25.014 | −1.093 | 20.763 | 1.00 | 15.53 | C |
| ATOM | 1789 | CB | ILE | B2115 | 25.589 | −0.191 | 19.664 | 1.00 | 16.82 | C |
| ATOM | 1790 | CG2 | ILE | B2115 | 26.983 | −0.702 | 19.253 | 1.00 | 14.25 | C |
| ATOM | 1791 | CG1 | ILE | B2115 | 24.649 | −0.168 | 18.461 | 1.00 | 17.22 | C |
| ATOM | 1792 | CD1 | ILE | B2115 | 25.020 | 0.874 | 17.423 | 1.00 | 18.67 | C |
| ATOM | 1793 | C | ILE | B2115 | 25.956 | −1.039 | 21.956 | 1.00 | 16.88 | C |
| ATOM | 1794 | O | ILE | B2115 | 26.080 | 0.006 | 22.614 | 1.00 | 16.02 | O |
| ATOM | 1795 | N | LEU | B2116 | 26.603 | −2.164 | 22.237 | 1.00 | 14.98 | N |
| ATOM | 1796 | CA | LEU | B2116 | 27.529 | −2.259 | 23.361 | 1.00 | 16.57 | C |
| ATOM | 1797 | CB | LEU | B2116 | 27.365 | −3.605 | 24.067 | 1.00 | 16.24 | C |
| ATOM | 1798 | CG | LEU | B2116 | 25.945 | −3.870 | 24.578 | 1.00 | 16.49 | C |
| ATOM | 1799 | CD1 | LEU | B2116 | 25.872 | −5.256 | 25.197 | 1.00 | 15.73 | C |
| ATOM | 1800 | CD2 | LEU | B2116 | 25.563 | −2.803 | 25.591 | 1.00 | 15.00 | C |
| ATOM | 1801 | C | LEU | B2116 | 28.945 | −2.121 | 22.852 | 1.00 | 17.64 | C |
| ATOM | 1802 | O | LEU | B2116 | 29.297 | −2.703 | 21.827 | 1.00 | 17.88 | O |
| ATOM | 1803 | N | GLU | B2117 | 29.763 | −1.373 | 23.584 | 1.00 | 19.16 | N |
| ATOM | 1804 | CA | GLU | B2117 | 31.132 | −1.136 | 23.166 | 1.00 | 22.44 | C |
| ATOM | 1805 | CB | GLU | B2117 | 31.199 | 0.229 | 22.473 | 1.00 | 21.55 | C |
| ATOM | 1806 | CG | GLU | B2117 | 32.570 | 0.672 | 22.003 | 1.00 | 26.09 | C |
| ATOM | 1807 | CD | GLU | B2117 | 32.563 | 2.110 | 21.477 | 1.00 | 26.21 | C |
| ATOM | 1808 | OE1 | GLU | B2117 | 31.958 | 2.997 | 22.132 | 1.00 | 24.17 | O |
| ATOM | 1809 | OE2 | GLU | B2117 | 33.170 | 2.355 | 20.412 | 1.00 | 27.59 | O |
| ATOM | 1810 | C | GLU | B2117 | 32.116 | −1.191 | 24.335 | 1.00 | 24.52 | C |
| ATOM | 1811 | O | GLU | B2117 | 31.829 | −0.711 | 25.432 | 1.00 | 21.96 | O |
| ATOM | 1812 | N | GLU | B2118 | 33.266 | −1.806 | 24.084 | 1.00 | 26.38 | N |
| ATOM | 1813 | CA | GLU | B2118 | 34.331 | −1.920 | 25.070 | 1.00 | 31.02 | C |
| ATOM | 1814 | CB | GLU | B2118 | 34.781 | −3.373 | 25.209 | 1.00 | 33.06 | C |
| ATOM | 1815 | CG | GLU | B2118 | 33.888 | −4.220 | 26.082 | 1.00 | 38.01 | C |
| ATOM | 1816 | CD | GLU | B2118 | 34.094 | −3.939 | 27.552 | 1.00 | 42.42 | C |
| ATOM | 1817 | OE1 | GLU | B2118 | 33.776 | −2.816 | 27.998 | 1.00 | 44.59 | O |
| ATOM | 1818 | OE2 | GLU | B2118 | 34.584 | −4.845 | 28.262 | 1.00 | 45.87 | O |
| ATOM | 1819 | C | GLU | B2118 | 35.482 | −1.091 | 24.533 | 1.00 | 32.66 | C |
| ATOM | 1820 | O | GLU | B2118 | 35.880 | −0.108 | 25.197 | 1.00 | 35.23 | O |
| ATOM | 1821 | OXT | GLU | B2118 | 35.958 | −1.442 | 23.431 | 1.00 | 32.94 | O |
| TER | 1822 | | GLU | B2118 | | | | | | |
| ATOM | 1823 | CB | MET | C3003 | 37.039 | 0.395 | 12.081 | 1.00 | 32.22 | C |
| ATOM | 1824 | CG | MET | C3003 | 37.297 | −0.447 | 10.845 | 1.00 | 36.68 | C |
| ATOM | 1825 | SD | MET | C3003 | 38.483 | 0.306 | 9.716 | 1.00 | 46.15 | S |
| ATOM | 1826 | CE | MET | C3003 | 37.454 | 1.490 | 8.876 | 1.00 | 42.14 | C |
| ATOM | 1827 | C | MET | C3003 | 34.864 | −0.764 | 12.331 | 1.00 | 25.86 | C |
| ATOM | 1828 | O | MET | C3003 | 34.821 | −1.903 | 11.864 | 1.00 | 26.11 | O |
| ATOM | 1829 | N | MET | C3003 | 36.772 | −1.388 | 13.778 | 1.00 | 27.93 | N |
| ATOM | 1830 | CA | MET | C3003 | 36.090 | −0.264 | 13.082 | 1.00 | 27.91 | C |
| ATOM | 1831 | N | ILE | C3004 | 33.866 | 0.097 | 12.219 | 1.00 | 23.54 | N |
| ATOM | 1832 | CA | ILE | C3004 | 32.655 | −0.247 | 11.500 | 1.00 | 21.79 | C |
| ATOM | 1833 | CB | ILE | C3004 | 31.503 | 0.697 | 11.890 | 1.00 | 22.07 | C |
| ATOM | 1834 | CG2 | ILE | C3004 | 30.259 | 0.377 | 11.063 | 1.00 | 20.08 | C |
| ATOM | 1835 | CG1 | ILE | C3004 | 31.212 | 0.560 | 13.391 | 1.00 | 21.69 | C |
| ATOM | 1836 | CD1 | ILE | C3004 | 30.181 | 1530 | 13.913 | 1.00 | 20.52 | C |
| ATOM | 1837 | C | ILE | C3004 | 32.942 | −0.109 | 10.005 | 1.00 | 22.42 | C |
| ATOM | 1838 | O | ILE | C3004 | 33.595 | 0.843 | 9.578 | 1.00 | 21.61 | O |
| ATOM | 1839 | N | VAL | C3005 | 32.483 | −1.071 | 9.212 | 1.00 | 21.57 | N |
| ATOM | 1840 | CA | VAL | C3005 | 32.692 | −0.999 | 7.777 | 1.00 | 22.56 | C |
| ATOM | 1841 | CB | VAL | C3005 | 33.600 | −2.145 | 7.265 | 1.00 | 24.27 | C |
| ATOM | 1842 | CG1 | VAL | C3005 | 34.954 | −2.087 | 7.975 | 1.00 | 23.54 | C |
| ATOM | 1843 | CG2 | VAL | C3005 | 32.924 | −3.487 | 7.478 | 1.00 | 25.12 | C |
| ATOM | 1844 | C | VAL | C3005 | 31.349 | −1.036 | 7.049 | 1.00 | 22.07 | C |
| ATOM | 1845 | O | VAL | C3005 | 31.297 | −0.988 | 5.823 | 1.00 | 22.90 | O |
| ATOM | 1846 | N | GLY | C3006 | 30.264 | −1.106 | 7.814 | 1.00 | 19.77 | N |
| ATOM | 1847 | CA | GLY | C3006 | 28.946 | −1.124 | 7.207 | 1.00 | 18.90 | C |
| ATOM | 1848 | C | GLY | C3006 | 27.840 | −1.111 | 8.240 | 1.00 | 18.66 | C |
| ATOM | 1849 | O | GLY | C3006 | 28.039 | −1.542 | 9.375 | 1.00 | 19.40 | O |
| ATOM | 1850 | N | HIS | C3007 | 26.679 | −0.595 | 7.849 | 1.00 | 17.60 | N |
| ATOM | 1851 | CA | HIS | C3007 | 25.517 | −0.544 | 8.726 | 1.00 | 17.25 | C |
| ATOM | 1852 | CB | HIS | C3007 | 25.541 | 0.709 | 9.615 | 1.00 | 17.61 | C |
| ATOM | 1853 | CG | HIS | C3007 | 24.315 | 0.867 | 10.469 | 1.00 | 17.57 | C |
| ATOM | 1854 | CD2 | HIS | C3007 | 23.615 | 1.967 | 10.834 | 1.00 | 17.24 | C |
| ATOM | 1855 | ND1 | HIS | C3007 | 23.686 | −0.203 | 11.072 | 1.00 | 17.16 | N |
| ATOM | 1856 | CE1 | HIS | C3007 | 22.651 | 0.231 | 11.771 | 1.00 | 17.81 | C |
| ATOM | 1857 | NE2 | HIS | C3007 | 22.585 | 1.545 | 11.644 | 1.00 | 18.60 | N |
| ATOM | 1858 | C | HIS | C3007 | 24.264 | −0.540 | 7.867 | 1.00 | 17.20 | C |
| ATOM | 1859 | O | HIS | C3007 | 24.138 | 0.249 | 6.924 | 1.00 | 17.44 | O |
| ATOM | 1860 | N | GLY | C3008 | 23.335 | −1.427 | 8.187 | 1.00 | 16.34 | N |
| ATOM | 1861 | CA | GLY | C3008 | 22.111 | −1.484 | 7.416 | 1.00 | 18.39 | C |
| ATOM | 1862 | C | GLY | C3008 | 20.925 | −1.924 | 8.242 | 1.00 | 19.04 | C |
| ATOM | 1863 | O | GLY | C3008 | 21.060 | −2.752 | 9.145 | 1.00 | 19.03 | O |
| ATOM | 1864 | N | ILE | C3009 | 19.761 | −1.352 | 7.944 | 1.00 | 18.83 | N |
| ATOM | 1865 | CA | ILE | C3009 | 18.542 | −1.718 | 8.640 | 1.00 | 19.42 | C |
| ATOM | 1866 | CB | ILE | C3009 | 18.047 | −0.598 | 9.594 | 1.00 | 18.51 | C |
| ATOM | 1867 | CG2 | ILE | C3009 | 19.197 | −0.150 | 10.493 | 1.00 | 20.36 | C |
| ATOM | 1868 | CG1 | ILE | C3009 | 17.505 | 0.585 | 8.797 | 1.00 | 17.23 | C |
| ATOM | 1869 | CD1 | ILE | C3009 | 16.778 | 1.592 | 9.643 | 1.00 | 13.66 | C |
| ATOM | 1870 | C | ILE | C3009 | 17.473 | −1.992 | 7.602 | 1.00 | 18.70 | C |
| ATOM | 1871 | O | ILE | C3009 | 17.647 | −1.685 | 6.427 | 1.00 | 15.95 | O |
| ATOM | 1872 | N | ASP | C3010 | 16.369 | −2.585 | 8.037 | 1.00 | 19.75 | N |
| ATOM | 1873 | CA | ASP | C3010 | 15.278 | −2.882 | 7.129 | 1.00 | 20.22 | C |
| ATOM | 1874 | CB | ASP | C3010 | 15.550 | −4.184 | 6.358 | 1.00 | 20.94 | C |
| ATOM | 1875 | CG | ASP | C3010 | 14.474 | −4.487 | 5.310 | 1.00 | 22.14 | C |
| ATOM | 1876 | OD1 | ASP | C3010 | 14.480 | −3.844 | 4.236 | 1.00 | 22.44 | O |
| ATOM | 1877 | OD2 | ASP | C3010 | 13.617 | −5.363 | 5.561 | 1.00 | 20.92 | O |
| ATOM | 1878 | C | ASP | C3010 | 14.007 | −3.047 | 7.932 | 1.00 | 21.28 | C |
| ATOM | 1879 | O | ASP | C3010 | 14.042 | −3.500 | 9.078 | 1.00 | 21.26 | O |
| ATOM | 1880 | N | ILE | C3011 | 12.893 | −2.646 | 7.333 | 1.00 | 19.66 | N |
| ATOM | 1881 | CA | ILE | C3011 | 11.588 | −2.813 | 7.949 | 1.00 | 20.09 | C |
| ATOM | 1882 | CB | ILE | C3011 | 10.959 | −1.476 | 8.400 | 1.00 | 20.39 | C |
| ATOM | 1883 | CG2 | ILE | C3011 | 10.910 | −0.478 | 7.234 | 1.00 | 16.27 | C |
| ATOM | 1884 | CG1 | ILE | C3011 | 9.561 | −1.753 | 8.971 | 1.00 | 20.84 | C |
| ATOM | 1885 | CD1 | ILE | C3011 | 9.081 | −0.712 | 9.969 | 1.00 | 21.63 | C |
| ATOM | 1886 | C | ILE | C3011 | 10.777 | −3.440 | 6.825 | 1.00 | 21.21 | C |
| ATOM | 1887 | O | ILE | C3011 | 10.866 | −3.006 | 5.678 | 1.00 | 21.49 | O |
| ATOM | 1888 | N | GLU | C3012 | 10.018 | −4.481 | 7.140 | 1.00 | 21.79 | N |
| ATOM | 1889 | CA | GLU | C3012 | 9.249 | −5.174 | 6.120 | 1.00 | 23.44 | C |
| ATOM | 1890 | CB | GLU | C3012 | 10.024 | −6.415 | 5.656 | 1.00 | 24.63 | C |
| ATOM | 1891 | CG | GLU | C3012 | 9.269 | −7.309 | 4.667 | 1.00 | 27.74 | C |
| ATOM | 1892 | CD | GLU | C3012 | 9.351 | −6.812 | 3.237 | 1.00 | 27.70 | C |
| ATOM | 1893 | OE1 | GLU | C3012 | 9.663 | −5.626 | 3.032 | 1.00 | 28.89 | O |
| ATOM | 1894 | OE2 | GLU | C3012 | 9.091 | −7.612 | 2.314 | 1.00 | 31.17 | O |
| ATOM | 1895 | C | GLU | C3012 | 7.873 | −5.583 | 6.625 | 1.00 | 23.57 | C |
| ATOM | 1896 | O | GLU | C3012 | 7.750 | −6.258 | 7.647 | 1.00 | 22.12 | O |
| ATOM | 1897 | N | GLU | C3013 | 6.840 | −5.170 | 5.904 | 1.00 | 23.45 | N |
| ATOM | 1898 | CA | GLU | C3013 | 5.491 | −5.519 | 6.286 | 1.00 | 24.76 | C |
| ATOM | 1899 | CB | GLU | C3013 | 4.479 | −4.613 | 5.571 | 1.00 | 29.22 | C |
| ATOM | 1900 | CG | GLU | C3013 | 3.662 | −3.731 | 6.531 | 1.00 | 33.17 | C |
| ATOM | 1901 | CD | GLU | C3013 | 3.915 | −2.242 | 6.349 | 1.00 | 38.85 | C |
| ATOM | 1902 | OE1 | GLU | C3013 | 3.660 | −1.719 | 5.238 | 1.00 | 44.44 | O |
| ATOM | 1903 | OE2 | GLU | C3013 | 4.359 | −1.586 | 7.318 | 1.00 | 38.04 | O |
| ATOM | 1904 | C | GLU | C3013 | 5.238 | −6.993 | 5.963 | 1.00 | 24.30 | C |
| ATOM | 1905 | O | GLU | C3013 | 5.643 | −7.502 | 4.910 | 1.00 | 21.31 | O |
| ATOM | 1906 | N | LEU | C3014 | 4.599 | −7.685 | 6.898 | 1.00 | 22.97 | N |
| ATOM | 1907 | CA | LEU | C3014 | 4.287 | −9.092 | 6.712 | 1.00 | 26.19 | C |
| ATOM | 1908 | CB | LEU | C3014 | 3.531 | −9.613 | 7.939 | 1.00 | 28.12 | C |
| ATOM | 1909 | CG | LEU | C3014 | 4.251 | −10.630 | 8.829 | 1.00 | 31.98 | C |
| ATOM | 1910 | CD1 | LEU | C3014 | 5.647 | −10.144 | 9.191 | 1.00 | 31.03 | C |
| ATOM | 1911 | CD2 | LEU | C3014 | 3.414 | −10.868 | 10.083 | 1.00 | 33.69 | C |
| ATOM | 1912 | C | LEU | C3014 | 3.453 | −9.308 | 5.439 | 1.00 | 25.44 | C |
| ATOM | 1913 | O | LEU | C3014 | 3.554 | −10.345 | 4.793 | 1.00 | 25.78 | O |
| ATOM | 1914 | N | ALA | C3015 | 2.644 | −8.320 | 5.073 | 1.00 | 24.83 | N |
| ATOM | 1915 | CA | ALA | C3015 | 1.801 | −8.438 | 3.879 | 1.00 | 27.12 | C |
| ATOM | 1916 | CB | ALA | C3015 | 0.915 | −7.206 | 3.747 | 1.00 | 27.24 | C |
| ATOM | 1917 | C | ALA | C3015 | 2.592 | −8.659 | 2.584 | 1.00 | 27.01 | C |
| ATOM | 1918 | O | ALA | C3015 | 2.215 | −9.497 | 1.761 | 1.00 | 26.98 | O |
| ATOM | 1919 | N | SER | C3016 | 3.683 | −7.916 | 2.399 | 1.00 | 26.19 | N |
| ATOM | 1920 | CA | SER | C3016 | 4.498 | −8.062 | 1.192 | 1.00 | 27.18 | C |
| ATOM | 1921 | CB | SER | C3016 | 5.706 | −7.117 | 1.232 | 1.00 | 27.62 | C |
| ATOM | 1922 | OG | SER | C3016 | 5.324 | −5.799 | 1.573 | 1.00 | 28.51 | O |
| ATOM | 1923 | C | SER | C3016 | 4.997 | −9.502 | 1.072 | 1.00 | 27.53 | C |
| ATOM | 1924 | O | SER | C3016 | 5.069 | −10.062 | −0.027 | 1.00 | 26.07 | O |
| ATOM | 1925 | N | ILE | C3017 | 5.343 | −10.094 | 2.212 | 1.00 | 26.80 | N |
| ATOM | 1926 | CA | ILE | C3017 | 5.841 | −11.464 | 2.248 | 1.00 | 25.88 | C |
| ATOM | 1927 | CB | ILE | C3017 | 6.519 | −11.771 | 3.613 | 1.00 | 25.28 | C |
| ATOM | 1928 | CG2 | ILE | C3017 | 6.909 | −13.241 | 3.688 | 1.00 | 23.81 | C |
| ATOM | 1929 | CG1 | ILE | C3017 | 7.739 | −10.863 | 3.808 | 1.00 | 24.39 | C |
| ATOM | 1930 | CD1 | ILE | C3017 | 8.815 | −11.007 | 2.735 | 1.00 | 24.49 | C |
| ATOM | 1931 | C | ILE | C3017 | 4.698 | −12.456 | 2.019 | 1.00 | 26.06 | C |
| ATOM | 1932 | O | ILE | C3017 | 4.830 | −13.408 | 1.253 | 1.00 | 26.57 | O |
| ATOM | 1933 | N | GLU | C3018 | 3.576 | −12.235 | 2.690 | 1.00 | 26.59 | N |
| ATOM | 1934 | CA | GLU | C3018 | 2.429 | −13.122 | 2.534 | 1.00 | 28.88 | C |
| ATOM | 1935 | CB | GLU | C3018 | 1.284 | −12.645 | 3.427 | 1.00 | 29.66 | C |
| ATOM | 1936 | CG | GLU | C3018 | 1.688 | −12.504 | 4.884 | 1.00 | 32.78 | C |
| ATOM | 1937 | CD | GLU | C3018 | 0.656 | −11.783 | 5.722 | 1.00 | 34.19 | C |
| ATOM | 1938 | OE1 | GLU | C3018 | −0.010 | −10.865 | 5.190 | 1.00 | 34.18 | O |
| ATOM | 1939 | OE2 | GLU | C3018 | 0.525 | −12.118 | 6.920 | 1.00 | 35.99 | O |
| ATOM | 1940 | C | GLU | C3018 | 1.983 | −13.163 | 1.067 | 1.00 | 29.36 | C |
| ATOM | 1941 | O | GLU | C3018 | 1.763 | −14.239 | 0.507 | 1.00 | 28.97 | O |
| ATOM | 1942 | N | SER | C3019 | 1.867 | −11.994 | 0.440 | 1.00 | 28.95 | N |
| ATOM | 1943 | CA | SER | C3019 | 1.454 | −11.931 | −0.957 | 1.00 | 29.90 | C |
| ATOM | 1944 | CB | SER | C3019 | 1.285 | −10.476 | −1.410 | 1.00 | 30.07 | C |
| ATOM | 1945 | OG | SER | C3019 | 2.542 | −9.866 | −1.632 | 1.00 | 33.15 | O |
| ATOM | 1946 | C | SER | C3019 | 2.489 | −12.623 | −1.837 | 1.00 | 30.43 | C |
| ATOM | 1947 | O | SER | C3019 | 2.138 | −13.362 | −2.755 | 1.00 | 30.07 | O |
| ATOM | 1948 | N | ALA | C3020 | 3.766 | −12.391 | −1.549 | 1.00 | 29.14 | N |
| ATOM | 1949 | CA | ALA | C3020 | 4.842 | −12.994 | −2.327 | 1.00 | 30.13 | C |
| ATOM | 1950 | CB | ALA | C3020 | 6.191 | −12.465 | −1.847 | 1.00 | 31.02 | C |
| ATOM | 1951 | C | ALA | C3020 | 4.834 | −14.527 | −2.269 | 1.00 | 30.69 | C |
| ATOM | 1952 | O | ALA | C3020 | 5.111 | −15.198 | −3.264 | 1.00 | 30.30 | O |
| ATOM | 1953 | N | VAL | C3021 | 4.530 | −15.082 | −1.103 | 1.00 | 30.94 | N |
| ATOM | 1954 | CA | VAL | C3021 | 4.501 | −16.532 | −0.959 | 1.00 | 33.03 | C |
| ATOM | 1955 | CB | VAL | C3021 | 4.172 | −16.950 | 0.489 | 1.00 | 32.04 | C |
| ATOM | 1956 | CG1 | VAL | C3021 | 3.982 | −18.465 | 0.565 | 1.00 | 31.51 | C |
| ATOM | 1957 | CG2 | VAL | C3021 | 5.292 | −16.521 | 1.414 | 1.00 | 31.14 | C |
| ATOM | 1958 | C | VAL | C3021 | 3.458 | −17.135 | −1.896 | 1.00 | 35.03 | C |
| ATOM | 1959 | O | VAL | C3021 | 3.736 | −18.094 | −2.616 | 1.00 | 36.27 | O |
| ATOM | 1960 | N | THR | C3022 | 2.263 | −16.552 | −1.889 | 1.00 | 36.26 | N |
| ATOM | 1961 | CA | THR | C3022 | 1.166 | −17.019 | −2.726 | 1.00 | 38.71 | C |
| ATOM | 1962 | CB | THR | C3022 | −0.142 | −16.274 | −2.371 | 1.00 | 38.89 | C |
| ATOM | 1963 | OG1 | THR | C3022 | −0.532 | −16.613 | −1.034 | 1.00 | 39.61 | O |
| ATOM | 1964 | CG2 | THR | C3022 | −1.262 | −16.654 | −3.336 | 1.00 | 37.67 | C |
| ATOM | 1965 | C | THR | C3022 | 1.458 | −16.836 | −4.214 | 1.00 | 40.67 | C |
| ATOM | 1966 | O | THR | C3022 | 1.325 | −17.772 | −5.005 | 1.00 | 40.21 | O |
| ATOM | 1967 | N | ARG | C3023 | 1.859 | −15.626 | −4.587 | 1.00 | 42.70 | N |
| ATOM | 1968 | CA | ARG | C3023 | 2.161 | −15.303 | −5.974 | 1.00 | 46.22 | C |
| ATOM | 1969 | CB | ARG | C3023 | 2.861 | −13.944 | −6.037 | 1.00 | 46.79 | C |
| ATOM | 1970 | CG | ARG | C3023 | 2.842 | −13.278 | −7.396 | 1.00 | 47.31 | C |
| ATOM | 1971 | CD | ARG | C3023 | 3.264 | −11.827 | −7.269 | 1.00 | 47.58 | C |
| ATOM | 1972 | NE | ARG | C3023 | 4.544 | −11.695 | −6.577 | 1.00 | 48.02 | N |
| ATOM | 1973 | CZ | ARG | C3023 | 4.741 | −10.924 | −5.511 | 1.00 | 48.91 | C |
| ATOM | 1974 | NH1 | ARG | C3023 | 3.736 | −10.214 | −5.008 | 1.00 | 46.67 | N |
| ATOM | 1975 | NH2 | ARG | C3023 | 5.943 | −10.861 | −4.948 | 1.00 | 48.13 | N |
| ATOM | 1976 | C | ARG | C3023 | 3.020 | −16.391 | −6.616 | 1.00 | 47.90 | C |
| ATOM | 1977 | O | ARG | C3023 | 2.684 | −16.906 | −7.681 | 1.00 | 49.41 | O |
| ATOM | 1978 | N | HIS | C3024 | 4.129 | −16.737 | −5.968 | 1.00 | 49.64 | N |
| ATOM | 1979 | CA | HIS | C3024 | 5.019 | −17.787 | −6.458 | 1.00 | 50.08 | C |
| ATOM | 1980 | CB | HIS | C3024 | 6.303 | −17.193 | −7.045 | 1.00 | 51.60 | C |
| ATOM | 1981 | CG | HIS | C3024 | 6.100 | −16.464 | −8.338 | 1.00 | 53.54 | C |
| ATOM | 1982 | CD2 | HIS | C3024 | 6.507 | −16.754 | −9.597 | 1.00 | 53.69 | C |
| ATOM | 1983 | ND1 | HIS | C3024 | 5.397 | −15.282 | −8.426 | 1.00 | 54.78 | N |
| ATOM | 1984 | CE1 | HIS | C3024 | 5.379 | −14.875 | −9.683 | 1.00 | 53.97 | C |
| ATOM | 1985 | NE2 | HIS | C3024 | 6.045 | −15.750 | −10.414 | 1.00 | 53.74 | N |
| ATOM | 1986 | C | HIS | C3024 | 5.370 | −18.720 | −5.304 | 1.00 | 50.32 | C |
| ATOM | 1987 | O | HIS | C3024 | 6.030 | −18.314 | −4.347 | 1.00 | 50.15 | O |
| ATOM | 1988 | N | GLU | C3025 | 4.929 | −19.971 | −5.394 | 1.00 | 49.54 | N |
| ATOM | 1989 | CA | GLU | C3025 | 5.197 | −20.937 | −4.335 | 1.00 | 49.18 | C |
| ATOM | 1990 | CB | GLU | C3025 | 4.507 | −22.270 | −4.632 | 1.00 | 52.19 | C |
| ATOM | 1991 | CG | GLU | C3025 | 4.778 | −23.331 | −3.575 | 1.00 | 55.07 | C |
| ATOM | 1992 | CD | GLU | C3025 | 4.269 | −24.694 | −3.972 | 1.00 | 57.92 | C |
| ATOM | 1993 | OE1 | GLU | C3025 | 3.033 | −24.870 | −4.053 | 1.00 | 58.55 | O |
| ATOM | 1994 | OE2 | GLU | C3025 | 5.111 | −25.590 | −4.206 | 1.00 | 59.67 | O |
| ATOM | 1995 | C | GLU | C3025 | 6.685 | −21.182 | −4.118 | 1.00 | 46.90 | C |
| ATOM | 1996 | O | GLU | C3025 | 7.087 | −21.711 | −3.084 | 1.00 | 47.27 | O |
| ATOM | 1997 | N | GLY | C3026 | 7.502 | −20.801 | −5.092 | 1.00 | 44.54 | N |
| ATOM | 1998 | CA | GLY | C3026 | 8.933 | −20.997 | −4.956 | 1.00 | 41.90 | C |
| ATOM | 1999 | C | GLY | C3026 | 9.636 | −19.811 | −4.320 | 1.00 | 40.26 | C |
| ATOM | 2000 | O | GLY | C3026 | 10.867 | −19.768 | −4.265 | 1.00 | 39.86 | O |
| ATOM | 2001 | N | PHE | C3027 | 8.860 | −18.842 | −3.841 | 1.00 | 37.59 | N |
| ATOM | 2002 | CA | PHE | C3027 | 9.434 | −17.655 | −3.217 | 1.00 | 35.31 | C |
| ATOM | 2003 | CB | PHE | C3027 | 8.323 | −16.715 | −2.738 | 1.00 | 34.09 | C |
| ATOM | 2004 | CG | PHE | C3027 | 8.832 | −15.491 | −2.031 | 1.00 | 33.77 | C |
| ATOM | 2005 | CD1 | PHE | C3027 | 9.633 | −14.569 | −2.700 | 1.00 | 33.43 | C |
| ATOM | 2006 | CD2 | PHE | C3027 | 8.543 | −15.277 | −0.687 | 1.00 | 32.68 | C |
| ATOM | 2007 | CE1 | PHE | C3027 | 10.141 | −13.450 | −2.042 | 1.00 | 32.28 | C |
| ATOM | 2008 | CE2 | PHE | C3027 | 9.045 | −14.163 | −0.016 | 1.00 | 33.06 | C |
| ATOM | 2009 | CZ | PHE | C3027 | 9.847 | −13.248 | −0.696 | 1.00 | 33.30 | C |
| ATOM | 2010 | C | PHE | C3027 | 10.331 | −18.032 | −2.042 | 1.00 | 34.03 | C |
| ATOM | 2011 | O | PHE | C3027 | 11.511 | −17.688 | −2.019 | 1.00 | 34.88 | O |
| ATOM | 2012 | N | ALA | C3028 | 9.767 | −18.743 | −1.071 | 1.00 | 33.39 | N |
| ATOM | 2013 | CA | ALA | C3028 | 10.516 | −19.159 | 0.109 | 1.00 | 34.32 | C |
| ATOM | 2014 | CB | ALA | C3028 | 9.651 | −20.053 | 0.987 | 1.00 | 33.64 | C |
| ATOM | 2015 | C | ALA | C3028 | 11.799 | −19.885 | −0.263 | 1.00 | 34.75 | C |
| ATOM | 2016 | O | ALA | C3028 | 12.873 | −19.572 | 0.257 | 1.00 | 34.90 | O |
| ATOM | 2017 | N | LYS | C3029 | 11.683 | −20.854 | −1.166 | 1.00 | 33.73 | N |
| ATOM | 2018 | CA | LYS | C3029 | 12.831 | −21.636 | −1.605 | 1.00 | 32.87 | C |
| ATOM | 2019 | CB | LYS | C3029 | 12.396 | −22.671 | −2.650 | 1.00 | 32.28 | C |
| ATOM | 2020 | CG | LYS | C3029 | 11.313 | −23.617 | −2.157 | 0.05 | 32.40 | C |
| ATOM | 2021 | CD | LYS | C3029 | 10.821 | −24.537 | −3.261 | 0.05 | 32.28 | C |
| ATOM | 2022 | CE | LYS | C3029 | 9.721 | −25.456 | −2.753 | 0.05 | 32.20 | C |
| ATOM | 2023 | NZ | LYS | C3029 | 9.160 | −26.313 | −3.833 | 0.05 | 32.15 | N |
| ATOM | 2024 | C | LYS | C3029 | 13.910 | −20.735 | −2.181 | 1.00 | 32.70 | C |
| ATOM | 2025 | O | LYS | C3029 | 15.100 | −21.016 | −2.048 | 1.00 | 33.44 | O |
| ATOM | 2026 | N | ARG | C3030 | 13.498 | −19.643 | −2.814 | 1.00 | 32.85 | N |
| ATOM | 2027 | CA | ARG | C3030 | 14.458 | −18.714 | −3.397 | 1.00 | 32.53 | C |
| ATOM | 2028 | CB | ARG | C3030 | 13.796 | −17.877 | −4.497 | 0.05 | 32.58 | C |
| ATOM | 2029 | CG | ARG | C3030 | 13.442 | −18.670 | −5.747 | 0.05 | 32.72 | C |
| ATOM | 2030 | CD | ARG | C3030 | 13.135 | −17.752 | −6.922 | 0.05 | 32.87 | C |
| ATOM | 2031 | NE | ARG | C3030 | 11.916 | −16.973 | −6.722 | 0.05 | 33.03 | N |
| ATOM | 2032 | CZ | ARG | C3030 | 10.691 | −17.490 | −6.720 | 0.05 | 33.10 | C |
| ATOM | 2033 | NH1 | ARG | C3030 | 10.517 | −18.791 | −6.908 | 0.05 | 33.16 | N |
| ATOM | 2034 | NH2 | ARG | C3030 | 9.639 | −16.705 | −6.531 | 0.05 | 33.04 | N |
| ATOM | 2035 | C | ARG | C3030 | 15.080 | −17.794 | −2.351 | 1.00 | 32.30 | C |
| ATOM | 2036 | O | ARG | C3030 | 16.242 | −17.408 | −2.470 | 1.00 | 32.85 | O |
| ATOM | 2037 | N | VAL | C3031 | 14.303 | −17.455 | −1.325 | 1.00 | 31.53 | N |
| ATOM | 2038 | CA | VAL | C3031 | 14.755 | −16.574 | −0.245 | 1.00 | 30.10 | C |
| ATOM | 2039 | CB | VAL | C3031 | 13.541 | −15.905 | 0.469 | 1.00 | 30.44 | C |
| ATOM | 2040 | CG1 | VAL | C3031 | 14.007 | −15.140 | 1.701 | 1.00 | 31.92 | C |
| ATOM | 2041 | CG2 | VAL | C3031 | 12.824 | −14.967 | −0.484 | 1.00 | 31.35 | C |
| ATOM | 2042 | C | VAL | C3031 | 15.576 | −17.284 | 0.836 | 1.00 | 29.72 | C |
| ATOM | 2043 | O | VAL | C3031 | 16.475 | −16.686 | 1.439 | 1.00 | 29.40 | O |
| ATOM | 2044 | N | LEU | C3032 | 15.259 | −18.554 | 1.076 | 1.00 | 27.31 | N |
| ATOM | 2045 | CA | LEU | C3032 | 15.911 | −19.338 | 2.119 | 1.00 | 25.97 | C |
| ATOM | 2046 | CB | LEU | C3032 | 14.839 | −19.917 | 3.047 | 1.00 | 24.74 | C |
| ATOM | 2047 | CG | LEU | C3032 | 13.782 | −18.943 | 3.572 | 1.00 | 22.82 | C |
| ATOM | 2048 | CD1 | LEU | C3032 | 12.717 | −19.708 | 4.361 | 1.00 | 23.17 | C |
| ATOM | 2049 | CD2 | LEU | C3032 | 14.454 | −17.893 | 4.452 | 1.00 | 21.55 | C |
| ATOM | 2050 | C | LEU | C3032 | 16.793 | −20.485 | 1.630 | 1.00 | 27.28 | C |
| ATOM | 2051 | O | LEU | C3032 | 16.447 | −21.187 | 0.676 | 1.00 | 28.34 | O |
| ATOM | 2052 | N | THR | C3033 | 17.924 | −20.676 | 2.307 | 1.00 | 26.40 | N |
| ATOM | 2053 | CA | THR | C3033 | 18.847 | −21.756 | 1.984 | 1.00 | 27.30 | C |
| ATOM | 2054 | CB | THR | C3033 | 20.149 | −21.662 | 2.795 | 1.00 | 25.69 | C |
| ATOM | 2055 | OG1 | THR | C3033 | 19.845 | −21.820 | 4.186 | 1.00 | 26.34 | O |
| ATOM | 2056 | CG2 | THR | C3033 | 20.835 | −20.323 | 2.574 | 1.00 | 26.77 | C |
| ATOM | 2057 | C | THR | C3033 | 18.170 | −23.062 | 2.385 | 1.00 | 27.70 | C |
| ATOM | 2058 | O | THR | C3033 | 17.061 | −23.061 | 2.920 | 1.00 | 28.12 | O |
| ATOM | 2059 | N | ALA | C3034 | 18.853 | −24.174 | 2.145 | 1.00 | 28.90 | N |
| ATOM | 2060 | CA | ALA | C3034 | 18.314 | −25.484 | 2.493 | 1.00 | 29.05 | C |
| ATOM | 2061 | CB | ALA | C3034 | 19.283 | −26.572 | 2.062 | 1.00 | 29.42 | C |
| ATOM | 2062 | C | ALA | C3034 | 18.047 | −25.594 | 3.990 | 1.00 | 28.60 | C |
| ATOM | 2063 | O | ALA | C3034 | 16.965 | −26.008 | 4.403 | 1.00 | 29.51 | O |
| ATOM | 2064 | N | LEU | C3035 | 19.029 | −25.218 | 4.805 | 1.00 | 28.04 | N |
| ATOM | 2065 | CA | LEU | C3035 | 18.869 | −25.310 | 6.249 | 1.00 | 27.61 | C |
| ATOM | 2066 | CB | LEU | C3035 | 20.187 | −24.964 | 6.952 | 1.00 | 29.31 | C |
| ATOM | 2067 | CG | LEU | C3035 | 20.522 | −25.809 | 8.190 | 1.00 | 31.59 | C |
| ATOM | 2068 | CD1 | LEU | C3035 | 20.472 | −27.297 | 7.821 | 1.00 | 32.29 | C |
| ATOM | 2069 | CD2 | LEU | C3035 | 21.902 | −25.438 | 8.727 | 1.00 | 30.00 | C |
| ATOM | 2070 | C | LEU | C3035 | 17.740 | −24.410 | 6.747 | 1.00 | 27.52 | C |
| ATOM | 2071 | O | LEU | C3035 | 16.922 | −24.832 | 7.571 | 1.00 | 26.23 | O |
| ATOM | 2072 | N | GLU | C3036 | 17.688 | −23.176 | 6.247 | 1.00 | 27.39 | N |
| ATOM | 2073 | CA | GLU | C3036 | 16.636 | −22.240 | 6.649 | 1.00 | 27.23 | C |
| ATOM | 2074 | CB | GLU | C3036 | 16.849 | −20.877 | 5.981 | 1.00 | 25.56 | C |
| ATOM | 2075 | CG | GLU | C3036 | 17.834 | −19.978 | 6.707 | 1.00 | 22.65 | C |
| ATOM | 2076 | CD | GLU | C3036 | 18.256 | −18.777 | 5.880 | 1.00 | 23.21 | C |
| ATOM | 2077 | OE1 | GLU | C3036 | 18.735 | −17.784 | 6.469 | 1.00 | 22.56 | O |
| ATOM | 2078 | OE2 | GLU | C3036 | 18.124 | −18.825 | 4.638 | 1.00 | 24.80 | O |
| ATOM | 2079 | C | GLU | C3036 | 15.258 | −22.793 | 6.278 | 1.00 | 28.00 | C |
| ATOM | 2080 | O | GLU | C3036 | 14.306 | −22.668 | 7.047 | 1.00 | 27.30 | O |
| ATOM | 2081 | N | MET | C3037 | 15.160 | −23.401 | 5.098 | 1.00 | 28.24 | N |
| ATOM | 2082 | CA | MET | C3037 | 13.897 | −23.979 | 4.641 | 1.00 | 30.24 | C |
| ATOM | 2083 | CB | MET | C3037 | 14.051 | −24.593 | 3.247 | 1.00 | 31.74 | C |
| ATOM | 2084 | CG | MET | C3037 | 13.885 | −23.605 | 2.102 | 1.00 | 32.56 | C |
| ATOM | 2085 | SD | MET | C3037 | 12.289 | −22.761 | 2.156 | 1.00 | 37.45 | S |
| ATOM | 2086 | CE | MET | C3037 | 11.153 | −24.108 | 1.753 | 1.00 | 36.07 | C |
| ATOM | 2087 | C | MET | C3037 | 13.411 | −25.049 | 5.608 | 1.00 | 31.16 | C |
| ATOM | 2088 | O | MET | C3037 | 12.222 | −25.124 | 5.920 | 1.00 | 30.55 | O |
| ATOM | 2089 | N | GLU | C3038 | 14.336 | −25.882 | 6.076 | 1.00 | 31.23 | N |
| ATOM | 2090 | CA | GLU | C3038 | 13.992 | −26.936 | 7.018 | 1.00 | 31.35 | C |
| ATOM | 2091 | CB | GLU | C3038 | 15.257 | −27.635 | 7.525 | 0.05 | 30.95 | C |
| ATOM | 2092 | CG | GLU | C3038 | 15.478 | −29.023 | 6.948 | 0.05 | 30.58 | C |
| ATOM | 2093 | CD | GLU | C3038 | 16.721 | −29.692 | 7.504 | 0.05 | 30.41 | C |
| ATOM | 2094 | OE1 | GLU | C3038 | 16.840 | −29.795 | 8.744 | 0.05 | 30.03 | O |
| ATOM | 2095 | OE2 | GLU | C3038 | 17.578 | −30.118 | 6.702 | 0.05 | 30.15 | O |
| ATOM | 2096 | C | GLU | C3038 | 13.235 | −26.330 | 8.191 | 1.00 | 31.53 | C |
| ATOM | 2097 | O | GLU | C3038 | 12.255 | −26.897 | 8.663 | 1.00 | 32.15 | O |
| ATOM | 2098 | N | ARG | C3039 | 13.697 | −25.176 | 8.664 | 1.00 | 32.00 | N |
| ATOM | 2099 | CA | ARG | C3039 | 13.046 | −24.496 | 9.781 | 1.00 | 33.21 | C |
| ATOM | 2100 | CB | ARG | C3039 | 13.928 | −23.353 | 10.289 | 1.00 | 34.11 | C |
| ATOM | 2101 | CG | ARG | C3039 | 13.317 | −22.548 | 11.434 | 1.00 | 37.68 | C |
| ATOM | 2102 | CD | ARG | C3039 | 13.189 | −23.377 | 12.704 | 1.00 | 39.59 | C |
| ATOM | 2103 | NE | ARG | C3039 | 12.565 | −22.626 | 13.791 | 1.00 | 41.92 | N |
| ATOM | 2104 | CZ | ARG | C3039 | 12.195 | −23.159 | 14.952 | 1.00 | 43.02 | C |
| ATOM | 2105 | NH1 | ARG | C3039 | 12.384 | −24.450 | 15.183 | 1.00 | 43.57 | N |
| ATOM | 2106 | NH2 | ARG | C3039 | 11.625 | −22.402 | 15.881 | 1.00 | 44.00 | N |
| ATOM | 2107 | C | ARG | C3039 | 11.692 | −23.939 | 9.339 | 1.00 | 32.70 | C |
| ATOM | 2108 | O | ARG | C3039 | 10.674 | −24.130 | 10.005 | 1.00 | 31.81 | O |
| ATOM | 2109 | N | PHE | C3040 | 11.699 | −23.240 | 8.210 | 1.00 | 32.86 | N |
| ATOM | 2110 | CA | PHE | C3040 | 10.489 | −22.646 | 7.653 | 1.00 | 33.52 | C |
| ATOM | 2111 | CB | PHE | C3040 | 10.778 | −22.128 | 6.242 | 1.00 | 32.08 | C |
| ATOM | 2112 | CG | PHE | C3040 | 9.569 | −21.604 | 5.528 | 1.00 | 32.50 | C |
| ATOM | 2113 | CD1 | PHE | C3040 | 9.012 | −20.380 | 5.880 | 1.00 | 31.27 | C |
| ATOM | 2114 | CD2 | PHE | C3040 | 8.980 | −22.340 | 4.503 | 1.00 | 32.76 | C |
| ATOM | 2115 | CE1 | PHE | C3040 | 7.882 | −19.895 | 5.222 | 1.00 | 31.92 | C |
| ATOM | 2116 | CE2 | PHE | C3040 | 7.851 | −21.865 | 3.839 | 1.00 | 33.27 | C |
| ATOM | 2117 | CZ | PHE | C3040 | 7.301 | −20.637 | 4.201 | 1.00 | 31.45 | C |
| ATOM | 2118 | C | PHE | C3040 | 9.367 | −23.680 | 7.595 | 1.00 | 33.45 | C |
| ATOM | 2119 | O | PHE | C3040 | 8.264 | −23.457 | 8.100 | 1.00 | 33.35 | O |
| ATOM | 2120 | N | THR | C3041 | 9.669 | −24.819 | 6.983 | 1.00 | 34.68 | N |
| ATOM | 2121 | CA | THR | C3041 | 8.706 | −25.898 | 6.828 | 1.00 | 35.57 | C |
| ATOM | 2122 | CB | THR | C3041 | 9.286 | −27.002 | 5.923 | 1.00 | 36.24 | C |
| ATOM | 2123 | OG1 | THR | C3041 | 9.517 | −26.464 | 4.616 | 1.00 | 38.04 | O |
| ATOM | 2124 | CG2 | THR | C3041 | 8.324 | −28.170 | 5.813 | 1.00 | 36.60 | C |
| ATOM | 2125 | C | THR | C3041 | 8.232 | −26.517 | 8.143 | 1.00 | 36.09 | C |
| ATOM | 2126 | O | THR | C3041 | 7.084 | −26.945 | 8.247 | 1.00 | 37.27 | O |
| ATOM | 2127 | N | SER | C3042 | 9.100 | −26.560 | 9.147 | 1.00 | 36.67 | N |
| ATOM | 2128 | CA | SER | C3042 | 8.718 | −27.142 | 10.427 | 1.00 | 37.38 | C |
| ATOM | 2129 | CB | SER | C3042 | 9.952 | −27.377 | 11.303 | 1.00 | 38.44 | C |
| ATOM | 2130 | OG | SER | C3042 | 10.391 | −26.172 | 11.904 | 1.00 | 39.31 | O |
| ATOM | 2131 | C | SER | C3042 | 7.744 | −26.230 | 11.157 | 1.00 | 37.81 | C |
| ATOM | 2132 | O | SER | C3042 | 7.010 | −26.671 | 12.042 | 1.00 | 38.07 | O |
| ATOM | 2133 | N | LEU | C3043 | 7.740 | −24.956 | 10.781 | 1.00 | 37.43 | N |
| ATOM | 2134 | CA | LEU | C3043 | 6.860 | −23.969 | 11.400 | 1.00 | 37.02 | C |
| ATOM | 2135 | CB | LEU | C3043 | 7.549 | −22.600 | 11.428 | 1.00 | 37.05 | C |
| ATOM | 2136 | CG | LEU | C3043 | 8.597 | −22.327 | 12.513 | 1.00 | 37.93 | C |
| ATOM | 2137 | CD1 | LEU | C3043 | 9.573 | −23.470 | 12.628 | 1.00 | 39.39 | C |
| ATOM | 2138 | CD2 | LEU | C3043 | 9.328 | −21.040 | 12.177 | 1.00 | 38.02 | C |
| ATOM | 2139 | C | LEU | C3043 | 5.526 | −23.851 | 10.666 | 1.00 | 37.04 | C |
| ATOM | 2140 | O | LEU | C3043 | 5.389 | −24.291 | 9.522 | 1.00 | 37.65 | O |
| ATOM | 2141 | N | LYS | C3044 | 4.542 | −23.250 | 11.325 | 1.00 | 36.12 | N |
| ATOM | 2142 | CA | LYS | C3044 | 3.236 | −23.075 | 10.707 | 1.00 | 35.63 | C |
| ATOM | 2143 | CB | LYS | C3044 | 2.313 | −24.246 | 11.072 | 1.00 | 37.63 | C |
| ATOM | 2144 | CG | LYS | C3044 | 1.985 | −24.356 | 12.555 | 1.00 | 40.09 | C |
| ATOM | 2145 | CD | LYS | C3044 | 0.926 | −25.432 | 12.813 | 1.00 | 42.38 | C |
| ATOM | 2146 | CE | LYS | C3044 | 1.397 | −26.813 | 12.365 | 1.00 | 42.85 | C |
| ATOM | 2147 | NZ | LYS | C3044 | 0.339 | −27.847 | 12.550 | 1.00 | 43.11 | N |
| ATOM | 2148 | C | LYS | C3044 | 2.584 | −21.753 | 11.111 | 1.00 | 33.57 | C |
| ATOM | 2149 | O | LYS | C3044 | 2.990 | −21.116 | 12.086 | 1.00 | 33.02 | O |
| ATOM | 2150 | N | GLY | C3045 | 1.579 | −21.345 | 10.339 | 1.00 | 31.64 | N |
| ATOM | 2151 | CA | GLY | C3045 | 0.860 | −20.115 | 10.618 | 1.00 | 29.90 | C |
| ATOM | 2152 | C | GLY | C3045 | 1.689 | −18.844 | 10.666 | 1.00 | 28.38 | C |
| ATOM | 2153 | O | GLY | C3045 | 2.643 | −18.676 | 9.915 | 1.00 | 28.13 | O |
| ATOM | 2154 | N | ARG | C3046 | 1.299 | −17.946 | 11.564 | 1.00 | 28.66 | N |
| ATOM | 2155 | CA | ARG | C3046 | 1.957 | −16.661 | 11.758 | 1.00 | 27.23 | C |
| ATOM | 2156 | CB | ARG | C3046 | 1.279 | −15.920 | 12.912 | 0.05 | 27.45 | C |
| ATOM | 2157 | CG | ARG | C3046 | −0.235 | −15.860 | 12.769 | 0.05 | 27.80 | C |
| ATOM | 2158 | CD | ARG | C3046 | −0.912 | −15.403 | 14.048 | 0.05 | 28.08 | C |
| ATOM | 2159 | NE | ARG | C3046 | −2.364 | −15.526 | 13.962 | 0.05 | 28.35 | N |
| ATOM | 2160 | CZ | ARG | C3046 | −3.199 | −15.249 | 14.959 | 0.05 | 28.51 | C |
| ATOM | 2161 | NH1 | ARG | C3046 | −4.506 | −15.394 | 14.792 | 0.05 | 28.59 | N |
| ATOM | 2162 | NH2 | ARG | C3046 | −2.727 | −14.831 | 16.127 | 0.05 | 28.63 | N |
| ATOM | 2163 | C | ARG | C3046 | 3.451 | −16.827 | 12.041 | 1.00 | 26.66 | C |
| ATOM | 2164 | O | ARG | C3046 | 4.276 | −16.065 | 11.537 | 1.00 | 23.83 | O |
| ATOM | 2165 | N | ARG | C3047 | 3.797 | −17.824 | 12.847 | 1.00 | 26.74 | N |
| ATOM | 2166 | CA | ARG | C3047 | 5.197 | −18.068 | 13.172 | 1.00 | 28.45 | C |
| ATOM | 2167 | CB | ARG | C3047 | 5.330 | −19.223 | 14.167 | 1.00 | 30.78 | C |
| ATOM | 2168 | CG | ARG | C3047 | 4.824 | −18.919 | 15.567 | 1.00 | 36.74 | C |
| ATOM | 2169 | CD | ARG | C3047 | 5.548 | −19.793 | 16.580 | 1.00 | 40.75 | C |
| ATOM | 2170 | NE | ARG | C3047 | 6.991 | −19.561 | 16.540 | 1.00 | 43.99 | N |
| ATOM | 2171 | CZ | ARG | C3047 | 7.894 | −20.341 | 17.126 | 1.00 | 45.84 | C |
| ATOM | 2172 | NH1 | ARG | C3047 | 7.508 | −21.417 | 17.803 | 1.00 | 47.05 | N |
| ATOM | 2173 | NH2 | ARG | C3047 | 9.184 | −20.046 | 17.036 | 1.00 | 46.26 | N |
| ATOM | 2174 | C | ARG | C3047 | 6.004 | −18.396 | 11.924 | 1.00 | 26.96 | C |
| ATOM | 2175 | O | ARG | C3047 | 7.171 | −18.019 | 11.809 | 1.00 | 27.18 | O |
| ATOM | 2176 | N | GLN | C3048 | 5.385 | −19.108 | 10.989 | 1.00 | 25.16 | N |
| ATOM | 2177 | CA | GLN | C3048 | 6.080 | −19.474 | 9.765 | 1.00 | 24.64 | C |
| ATOM | 2178 | CB | GLN | C3048 | 5.262 | −20.489 | 8.964 | 1.00 | 24.71 | C |
| ATOM | 2179 | CG | GLN | C3048 | 6.087 | −21.176 | 7.884 | 1.00 | 24.87 | C |
| ATOM | 2180 | CD | GLN | C3048 | 5.261 | −22.028 | 6.947 | 1.00 | 27.10 | C |
| ATOM | 2181 | OE1 | GLN | C3048 | 5.757 | −23.007 | 6.393 | 1.00 | 28.27 | O |
| ATOM | 2182 | NE2 | GLN | C3048 | 4.000 | −21.650 | 6.747 | 1.00 | 27.28 | N |
| ATOM | 2183 | C | GLN | C3048 | 6.342 | −18.240 | 8.907 | 1.00 | 23.81 | C |
| ATOM | 2184 | O | GLN | C3048 | 7.441 | −18.064 | 8.376 | 1.00 | 22.00 | O |
| ATOM | 2185 | N | ILE | C3049 | 5.323 | −17.394 | 8.767 | 1.00 | 23.83 | N |
| ATOM | 2186 | CA | ILE | C3049 | 5.449 | −16.174 | 7.972 | 1.00 | 22.84 | C |
| ATOM | 2187 | CB | ILE | C3049 | 4.099 | −15.428 | 7.889 | 1.00 | 23.39 | C |
| ATOM | 2188 | CG2 | ILE | C3049 | 4.270 | −14.110 | 7.123 | 1.00 | 20.78 | C |
| ATOM | 2189 | CG1 | ILE | C3049 | 3.068 | −16.317 | 7.194 | 1.00 | 25.01 | C |
| ATOM | 2190 | CD1 | ILE | C3049 | 1.637 | −15.812 | 7.313 | 1.00 | 28.28 | C |
| ATOM | 2191 | C | ILE | C3049 | 6.492 | −15.254 | 8.596 | 1.00 | 22.76 | C |
| ATOM | 2192 | O | ILE | C3049 | 7.334 | −14.695 | 7.901 | 1.00 | 22.64 | O |
| ATOM | 2193 | N | GLU | C3050 | 6.426 | −15.119 | 9.917 | 1.00 | 23.06 | N |
| ATOM | 2194 | CA | GLU | C3050 | 7.347 | −14.287 | 10.685 | 1.00 | 23.40 | C |
| ATOM | 2195 | CB | GLU | C3050 | 7.023 | −14.436 | 12.169 | 1.00 | 27.99 | C |
| ATOM | 2196 | CG | GLU | C3050 | 7.336 | −13.229 | 13.014 | 1.00 | 34.34 | C |
| ATOM | 2197 | CD | GLU | C3050 | 6.633 | −11.997 | 12.501 | 1.00 | 37.30 | C |
| ATOM | 2198 | OE1 | GLU | C3050 | 7.273 | −11.233 | 11.752 | 1.00 | 38.54 | O |
| ATOM | 2199 | OE2 | GLU | C3050 | 5.437 | −11.807 | 12.825 | 1.00 | 38.13 | O |
| ATOM | 2200 | C | GLU | C3050 | 8.798 | −14.707 | 10.429 | 1.00 | 22.91 | C |
| ATOM | 2201 | O | GLU | C3050 | 9.675 | −13.863 | 10.212 | 1.00 | 19.78 | O |
| ATOM | 2202 | N | TYR | C3051 | 9.042 | −16.018 | 10.451 | 1.00 | 20.98 | N |
| ATOM | 2203 | CA | TYR | C3051 | 10.379 | −16.549 | 10.218 | 1.00 | 20.96 | C |
| ATOM | 2204 | CB | TYR | C3051 | 10.376 | −18.087 | 10.286 | 1.00 | 20.94 | C |
| ATOM | 2205 | CG | TYR | C3051 | 11.727 | −18.710 | 9.996 | 1.00 | 21.84 | C |
| ATOM | 2206 | CD1 | TYR | C3051 | 12.091 | −19.072 | 8.699 | 1.00 | 21.88 | C |
| ATOM | 2207 | CE1 | TYR | C3051 | 13.365 | −19.563 | 8.420 | 1.00 | 22.08 | C |
| ATOM | 2208 | CD2 | TYR | C3051 | 12.675 | −18.864 | 11.011 | 1.00 | 24.22 | C |
| ATOM | 2209 | CE2 | TYR | C3051 | 13.947 | −19.353 | 10.743 | 1.00 | 22.50 | C |
| ATOM | 2210 | CZ | TYR | C3051 | 14.286 | −19.695 | 9.447 | 1.00 | 23.28 | C |
| ATOM | 2211 | OH | TYR | C3051 | 15.557 | −20.142 | 9.181 | 1.00 | 26.17 | O |
| ATOM | 2212 | C | TYR | C3051 | 10.893 | −16.093 | 8.863 | 1.00 | 21.41 | C |
| ATOM | 2213 | O | TYR | C3051 | 11.973 | −15.508 | 8.764 | 1.00 | 20.67 | O |
| ATOM | 2214 | N | LEU | C3052 | 10.104 | −16.357 | 7.823 | 1.00 | 20.18 | N |
| ATOM | 2215 | CA | LEU | C3052 | 10.468 | −15.985 | 6.465 | 1.00 | 19.73 | C |
| ATOM | 2216 | CB | LEU | C3052 | 9.347 | −16.399 | 5.502 | 1.00 | 20.90 | C |
| ATOM | 2217 | CG | LEU | C3052 | 9.488 | −16.052 | 4.019 | 1.00 | 20.73 | C |
| ATOM | 2218 | CD1 | LEU | C3052 | 10.837 | −16.537 | 3.484 | 1.00 | 21.92 | C |
| ATOM | 2219 | CD2 | LEU | C3052 | 8.329 | −16.704 | 3.247 | 1.00 | 22.34 | C |
| ATOM | 2220 | C | LEU | C3052 | 10.725 | −14.487 | 6.365 | 1.00 | 18.62 | C |
| ATOM | 2221 | O | LEU | C3052 | 11.745 | −14.057 | 5.819 | 1.00 | 18.97 | O |
| ATOM | 2222 | N | ALA | C3053 | 9.805 | −13.697 | 6.912 | 1.00 | 18.23 | N |
| ATOM | 2223 | CA | ALA | C3053 | 9.921 | −12.238 | 6.888 | 1.00 | 19.28 | C |
| ATOM | 2224 | CB | ALA | C3053 | 8.670 | −11.611 | 7.489 | 1.00 | 19.78 | C |
| ATOM | 2225 | C | ALA | C3053 | 11.164 | −11.746 | 7.634 | 1.00 | 18.63 | C |
| ATOM | 2226 | O | ALA | C3053 | 11.835 | −10.815 | 7.191 | 1.00 | 17.69 | O |
| ATOM | 2227 | N | GLY | C3054 | 11.464 | −12.375 | 8.767 | 1.00 | 18.30 | N |
| ATOM | 2228 | CA | GLY | C3054 | 12.635 | −11.989 | 9.534 | 1.00 | 18.14 | C |
| ATOM | 2229 | C | GLY | C3054 | 13.915 | −12.303 | 8.776 | 1.00 | 19.55 | C |
| ATOM | 2230 | O | GLY | C3054 | 14.888 | −11.545 | 8.831 | 1.00 | 18.40 | O |
| ATOM | 2231 | N | ARG | C3055 | 13.927 | −13.421 | 8.060 | 1.00 | 18.30 | N |
| ATOM | 2232 | CA | ARG | C3055 | 15.112 | −13.783 | 7.298 | 1.00 | 19.48 | C |
| ATOM | 2233 | CB | ARG | C3055 | 15.021 | −15.239 | 6.839 | 1.00 | 20.53 | C |
| ATOM | 2234 | CG | ARG | C3055 | 15.815 | −16.213 | 7.709 | 1.00 | 22.48 | C |
| ATOM | 2235 | CD | ARG | C3055 | 15.450 | −16.093 | 9.181 | 1.00 | 26.13 | C |
| ATOM | 2236 | NE | ARG | C3055 | 16.291 | −16.945 | 10.019 | 1.00 | 28.05 | N |
| ATOM | 2237 | CZ | ARG | C3055 | 16.240 | −16.972 | 11.347 | 1.00 | 28.02 | C |
| ATOM | 2238 | NH1 | ARG | C3055 | 15.389 | −16.190 | 11.999 | 1.00 | 28.89 | N |
| ATOM | 2239 | NH2 | ARG | C3055 | 17.034 | −17.788 | 12.024 | 1.00 | 28.23 | N |
| ATOM | 2240 | C | ARG | C3055 | 15.283 | −12.849 | 6.108 | 1.00 | 20.40 | C |
| ATOM | 2241 | O | ARG | C3055 | 16.396 | −12.399 | 5.817 | 1.00 | 20.36 | O |
| ATOM | 2242 | N | TRP | C3056 | 14.182 | −12.547 | 5.424 | 1.00 | 20.72 | N |
| ATOM | 2243 | CA | TRP | C3056 | 14.234 | −11.641 | 4.280 | 1.00 | 22.18 | C |
| ATOM | 2244 | CB | TRP | C3056 | 12.837 | −11.486 | 3.655 | 1.00 | 23.99 | C |
| ATOM | 2245 | CG | TRP | C3056 | 12.716 | −10.303 | 2.719 | 1.00 | 29.31 | C |
| ATOM | 2246 | CD2 | TRP | C3056 | 12.723 | −10.335 | 1.289 | 1.00 | 30.25 | C |
| ATOM | 2247 | CE2 | TRP | C3056 | 12.609 | −9.001 | 0.840 | 1.00 | 32.03 | C |
| ATOM | 2248 | CE3 | TRP | C3056 | 12.821 | −11.362 | 0.342 | 1.00 | 33.18 | C |
| ATOM | 2249 | CD1 | TRP | C3056 | 12.598 | −8.984 | 3.070 | 1.00 | 30.82 | C |
| ATOM | 2250 | NE1 | TRP | C3056 | 12.532 | −8.198 | 1.947 | 1.00 | 33.62 | N |
| ATOM | 2251 | CZ2 | TRP | C3056 | 12.582 | −8.665 | −0.515 | 1.00 | 34.74 | C |
| ATOM | 2252 | CZ3 | TRP | C3056 | 12.795 | −11.029 | −1.010 | 1.00 | 34.82 | C |
| ATOM | 2253 | CH2 | TRP | C3056 | 12.679 | −9.690 | −1.425 | 1.00 | 36.26 | C |
| ATOM | 2254 | C | TRP | C3056 | 14.746 | −10.284 | 4.760 | 1.00 | 20.86 | C |
| ATOM | 2255 | O | TRP | C3056 | 15.608 | −9.670 | 4.134 | 1.00 | 18.98 | O |
| ATOM | 2256 | N | SER | C3057 | 14.206 | −9.835 | 5.885 | 1.00 | 18.95 | N |
| ATOM | 2257 | CA | SER | C3057 | 14.586 | −8.558 | 6.487 | 1.00 | 19.90 | C |
| ATOM | 2258 | CB | SER | C3057 | 13.755 | −8.321 | 7.752 | 1.00 | 21.78 | C |
| ATOM | 2259 | OG | SER | C3057 | 14.067 | −7.076 | 8.342 | 1.00 | 25.39 | O |
| ATOM | 2260 | C | SER | C3057 | 16.070 | −8.506 | 6.841 | 1.00 | 18.84 | C |
| ATOM | 2261 | O | SER | C3057 | 16.767 | −7.545 | 6.514 | 1.00 | 19.51 | O |
| ATOM | 2262 | N | ALA | C3058 | 16.550 | −9.545 | 7.509 | 1.00 | 18.85 | N |
| ATOM | 2263 | CA | ALA | C3058 | 17.949 | −9.611 | 7.917 | 1.00 | 18.94 | C |
| ATOM | 2264 | CB | ALA | C3058 | 18.182 | −10.854 | 8.765 | 1.00 | 16.86 | C |
| ATOM | 2265 | C | ALA | C3058 | 18.912 | −9.608 | 6.730 | 1.00 | 19.28 | C |
| ATOM | 2266 | O | ALA | C3058 | 19.955 | −8.946 | 6.765 | 1.00 | 19.47 | O |
| ATOM | 2267 | N | LYS | C3059 | 18.567 | −10.349 | 5.683 | 1.00 | 18.93 | N |
| ATOM | 2268 | CA | LYS | C3059 | 19.424 | −10.432 | 4.507 | 1.00 | 19.60 | C |
| ATOM | 2269 | CB | LYS | C3059 | 18.965 | −11.575 | 3.604 | 1.00 | 20.15 | C |
| ATOM | 2270 | CG | LYS | C3059 | 19.102 | −12.925 | 4.271 | 1.00 | 17.40 | C |
| ATOM | 2271 | CD | LYS | C3059 | 18.773 | −14.069 | 3.331 | 1.00 | 21.58 | C |
| ATOM | 2272 | CE | LYS | C3059 | 18.813 | −15.392 | 4.070 | 1.00 | 19.13 | C |
| ATOM | 2273 | NZ | LYS | C3059 | 18.667 | −16.545 | 3.158 | 1.00 | 24.92 | N |
| ATOM | 2274 | C | LYS | C3059 | 19.437 | −9.121 | 3.744 | 1.00 | 21.37 | C |
| ATOM | 2275 | O | LYS | C3059 | 20.457 | −8.734 | 3.165 | 1.00 | 22.73 | O |
| ATOM | 2276 | N | GLU | C3060 | 18.297 | −8.440 | 3.745 | 1.00 | 22.26 | N |
| ATOM | 2277 | CA | GLU | C3060 | 18.175 | −7.158 | 3.080 | 1.00 | 22.29 | C |
| ATOM | 2278 | CB | GLU | C3060 | 16.708 | −6.716 | 3.076 | 1.00 | 24.97 | C |
| ATOM | 2279 | CG | GLU | C3060 | 16.424 | −5.392 | 2.373 | 1.00 | 32.66 | C |
| ATOM | 2280 | CD | GLU | C3060 | 16.807 | −5.410 | 0.901 | 1.00 | 37.10 | C |
| ATOM | 2281 | OE1 | GLU | C3060 | 16.401 | −6.359 | 0.190 | 1.00 | 38.38 | O |
| ATOM | 2282 | OE2 | GLU | C3060 | 17.509 | −4.472 | 0.456 | 1.00 | 38.70 | O |
| ATOM | 2283 | C | GLU | C3060 | 19.042 | −6.172 | 3.864 | 1.00 | 20.94 | C |
| ATOM | 2284 | O | GLU | C3060 | 19.829 | −5.429 | 3.285 | 1.00 | 22.93 | O |
| ATOM | 2285 | N | ALA | C3061 | 18.907 | −6.178 | 5.186 | 1.00 | 19.68 | N |
| ATOM | 2286 | CA | ALA | C3061 | 19.698 | −5.282 | 6.024 | 1.00 | 20.17 | C |
| ATOM | 2287 | CB | ALA | C3061 | 19.373 | −5.515 | 7.499 | 1.00 | 19.49 | C |
| ATOM | 2288 | C | ALA | C3061 | 21.185 | −5.520 | 5.759 | 1.00 | 19.14 | C |
| ATOM | 2289 | O | ALA | C3061 | 21.953 | −4.576 | 5.572 | 1.00 | 18.81 | O |
| ATOM | 2290 | N | PHE | C3062 | 21.588 | −6.786 | 5.723 | 1.00 | 19.90 | N |
| ATOM | 2291 | CA | PHE | C3062 | 22.987 | −7.115 | 5.465 | 1.00 | 19.78 | C |
| ATOM | 2292 | CB | PHE | C3062 | 23.214 | −8.624 | 5.570 | 1.00 | 20.80 | C |
| ATOM | 2293 | CG | PHE | C3062 | 24.604 | −9.051 | 5.171 | 1.00 | 21.17 | C |
| ATOM | 2294 | CD1 | PHE | C3062 | 24.896 | −9.376 | 3.847 | 1.00 | 21.25 | C |
| ATOM | 2295 | CD2 | PHE | C3062 | 25.629 | −9.074 | 6.109 | 1.00 | 20.45 | C |
| ATOM | 2296 | CE1 | PHE | C3062 | 26.195 | −9.717 | 3.463 | 1.00 | 22.31 | C |
| ATOM | 2297 | CE2 | PHE | C3062 | 26.930 | −9.412 | 5.738 | 1.00 | 21.98 | C |
| ATOM | 2298 | CZ | PHE | C3062 | 27.211 | −9.733 | 4.410 | 1.00 | 22.06 | C |
| ATOM | 2299 | C | PHE | C3062 | 23.443 | −6.629 | 4.089 | 1.00 | 21.35 | C |
| ATOM | 2300 | O | PHE | C3062 | 24.526 | −6.067 | 3.951 | 1.00 | 20.77 | O |
| ATOM | 2301 | N | SER | C3063 | 22.612 | −6.843 | 3.074 | 1.00 | 22.17 | N |
| ATOM | 2302 | CA | SER | C3063 | 22.949 | −6.434 | 1.717 | 1.00 | 22.74 | C |
| ATOM | 2303 | CB | SER | C3063 | 21.851 | −6.873 | 0.754 | 1.00 | 23.43 | C |
| ATOM | 2304 | OG | SER | C3063 | 21.635 | −8.265 | 0.877 | 1.00 | 28.48 | O |
| ATOM | 2305 | C | SER | C3063 | 23.123 | −4.926 | 1.649 | 1.00 | 22.84 | C |
| ATOM | 2306 | O | SER | C3063 | 24.001 | −4.422 | 0.947 | 1.00 | 21.58 | O |
| ATOM | 2307 | N | LYS | C3064 | 22.277 | −4.205 | 2.377 | 1.00 | 21.35 | N |
| ATOM | 2308 | CA | LYS | C3064 | 22.370 | −2.755 | 2.406 | 1.00 | 21.55 | C |
| ATOM | 2309 | CB | LYS | C3064 | 21.111 | −2.168 | 3.055 | 1.00 | 22.13 | C |
| ATOM | 2310 | CG | LYS | C3064 | 19.841 | −2.416 | 2.238 | 1.00 | 21.86 | C |
| ATOM | 2311 | CD | LYS | C3064 | 18.588 | −1.884 | 2.929 | 1.00 | 22.40 | C |
| ATOM | 2312 | CE | LYS | C3064 | 18.653 | −0.375 | 3.120 | 1.00 | 22.07 | C |
| ATOM | 2313 | NZ | LYS | C3064 | 17.579 | 0.125 | 4.017 | 1.00 | 21.49 | N |
| ATOM | 2314 | C | LYS | C3064 | 23.639 | −2.341 | 3.168 | 1.00 | 21.95 | C |
| ATOM | 2315 | O | LYS | C3064 | 24.295 | −1.363 | 2.812 | 1.00 | 22.05 | O |
| ATOM | 2316 | N | ALA | C3065 | 23.994 | −3.104 | 4.197 | 1.00 | 20.93 | N |
| ATOM | 2317 | CA | ALA | C3065 | 25.193 | −2.818 | 4.983 | 1.00 | 23.70 | C |
| ATOM | 2318 | CB | ALA | C3065 | 25.254 | −3.729 | 6.200 | 1.00 | 22.08 | C |
| ATOM | 2319 | C | ALA | C3065 | 26.450 | −3.010 | 4.136 | 1.00 | 25.75 | C |
| ATOM | 2320 | O | ALA | C3065 | 27.420 | −2.278 | 4.297 | 1.00 | 25.29 | O |
| ATOM | 2321 | N | MET | C3066 | 26.433 | −4.008 | 3.255 | 1.00 | 27.80 | N |
| ATOM | 2322 | CA | MET | C3066 | 27.569 | −4.278 | 2.375 | 1.00 | 31.06 | C |
| ATOM | 2323 | CB | MET | C3066 | 27.386 | −5.616 | 1.649 | 1.00 | 30.03 | C |
| ATOM | 2324 | CG | MET | C3066 | 27.666 | −6.848 | 2.495 | 1.00 | 30.77 | C |
| ATOM | 2325 | SD | MET | C3066 | 29.395 | −6.944 | 3.059 | 1.00 | 31.09 | S |
| ATOM | 2326 | CE | MET | C3066 | 30.192 | −7.610 | 1.576 | 1.00 | 29.01 | C |
| ATOM | 2327 | C | MET | C3066 | 27.683 | −3.159 | 1.342 | 1.00 | 33.44 | C |
| ATOM | 2328 | O | MET | C3066 | 28.778 | −2.782 | 0.940 | 1.00 | 32.94 | O |
| ATOM | 2329 | N | GLY | C3067 | 26.539 | −2.634 | 0.918 | 1.00 | 36.57 | N |
| ATOM | 2330 | CA | GLY | C3067 | 26.542 | −1.565 | −0.059 | 1.00 | 40.47 | C |
| ATOM | 2331 | C | GLY | C3067 | 26.101 | −2.041 | −1.426 | 1.00 | 43.36 | C |
| ATOM | 2332 | O | GLY | C3067 | 26.602 | −1.573 | −2.447 | 1.00 | 44.87 | O |
| ATOM | 2333 | N | THR | C3068 | 25.161 | −2.976 | −1.447 | 0.05 | 44.27 | N |
| ATOM | 2334 | CA | THR | C3068 | 24.650 | −3.506 | −2.701 | 0.05 | 45.40 | C |
| ATOM | 2335 | CB | THR | C3068 | 25.521 | −4.680 | −3.201 | 0.05 | 45.40 | C |
| ATOM | 2336 | OG1 | THR | C3068 | 25.018 | −5.147 | −4.459 | 0.05 | 45.48 | O |
| ATOM | 2337 | CG2 | THR | C3068 | 25.518 | −5.821 | −2.193 | 0.05 | 45.43 | C |
| ATOM | 2338 | C | THR | C3068 | 23.212 | −3.973 | −2.507 | 0.05 | 46.15 | C |
| ATOM | 2339 | O | THR | C3068 | 22.452 | −3.355 | −1.761 | 0.05 | 46.24 | O |
| ATOM | 2340 | N | GLY | C3069 | 22.839 | −5.058 | −3.175 | 0.05 | 47.05 | N |
| ATOM | 2341 | CA | GLY | C3069 | 21.488 | −5.567 | −3.043 | 0.05 | 48.19 | C |
| ATOM | 2342 | C | GLY | C3069 | 21.341 | −6.984 | −3.555 | 0.05 | 48.95 | C |
| ATOM | 2343 | O | GLY | C3069 | 22.322 | −7.627 | −3.927 | 0.05 | 49.00 | O |
| ATOM | 2344 | N | ILE | C3070 | 20.106 | −7.471 | −3.569 | 0.05 | 49.77 | N |
| ATOM | 2345 | CA | ILE | C3070 | 19.809 | −8.817 | −4.038 | 0.05 | 50.58 | C |
| ATOM | 2346 | CB | ILE | C3070 | 18.311 | −9.155 | −3.800 | 0.05 | 50.80 | C |
| ATOM | 2347 | CG2 | ILE | C3070 | 17.434 | −8.193 | −4.584 | 0.05 | 51.01 | C |
| ATOM | 2348 | CG1 | ILE | C3070 | 18.011 | −10.600 | −4.205 | 0.05 | 51.05 | C |
| ATOM | 2349 | CD1 | ILE | C3070 | 18.852 | −11.631 | −3.486 | 0.05 | 51.19 | C |
| ATOM | 2350 | C | ILE | C3070 | 20.140 | −8.927 | −5.528 | 0.05 | 50.91 | C |
| ATOM | 2351 | O | ILE | C3070 | 20.642 | −7.977 | −6.126 | 0.05 | 50.98 | O |
| ATOM | 2352 | N | SER | C3071 | 19.868 | −10.093 | −6.108 | 0.05 | 51.31 | N |
| ATOM | 2353 | CA | SER | C3071 | 20.112 | −10.372 | −7.522 | 0.05 | 51.63 | C |
| ATOM | 2354 | CB | SER | C3071 | 19.393 | −9.349 | −8.411 | 0.05 | 51.73 | C |
| ATOM | 2355 | OG | SER | C3071 | 20.018 | −8.080 | −8.363 | 0.05 | 51.85 | O |
| ATOM | 2356 | C | SER | C3071 | 21.592 | −10.420 | −7.891 | 0.05 | 51.76 | C |
| ATOM | 2357 | O | SER | C3071 | 21.967 | −11.053 | −8.878 | 0.05 | 51.87 | O |
| ATOM | 2358 | N | LYS | C3072 | 22.433 | −9.755 | −7.106 | 0.05 | 51.86 | N |
| ATOM | 2359 | CA | LYS | C3072 | 23.867 | −9.751 | −7.374 | 0.05 | 51.85 | C |
| ATOM | 2360 | CB | LYS | C3072 | 24.396 | −8.312 | −7.449 | 0.05 | 52.23 | C |
| ATOM | 2361 | CG | LYS | C3072 | 24.392 | −7.543 | −6.131 | 0.05 | 52.70 | C |
| ATOM | 2362 | CD | LYS | C3072 | 25.580 | −7.907 | −5.245 | 0.05 | 53.07 | C |
| ATOM | 2363 | CE | LYS | C3072 | 26.905 | −7.581 | −5.920 | 0.05 | 53.33 | C |
| ATOM | 2364 | NZ | LYS | C3072 | 27.022 | −6.134 | −6.253 | 0.05 | 53.48 | N |
| ATOM | 2365 | C | LYS | C3072 | 24.606 | −10.529 | −6.292 | 0.05 | 51.55 | C |
| ATOM | 2366 | O | LYS | C3072 | 25.643 | −11.140 | −6.550 | 0.05 | 51.64 | O |
| ATOM | 2367 | N | LEU | C3073 | 24.062 | −10.500 | −5.080 | 0.05 | 51.11 | N |
| ATOM | 2368 | CA | LEU | C3073 | 24.660 | −11.204 | −3.953 | 0.05 | 50.57 | C |
| ATOM | 2369 | CB | LEU | C3073 | 24.615 | −10.328 | −2.699 | 0.05 | 50.79 | C |
| ATOM | 2370 | CG | LEU | C3073 | 25.349 | −10.863 | −1.467 | 0.05 | 50.92 | C |
| ATOM | 2371 | CD1 | LEU | C3073 | 26.830 | −11.010 | −1.783 | 0.05 | 51.03 | C |
| ATOM | 2372 | CD2 | LEU | C3073 | 25.145 | −9.917 | −0.294 | 0.05 | 50.94 | C |
| ATOM | 2373 | C | LEU | C3073 | 23.893 | −12.498 | −3.707 | 0.05 | 50.00 | C |
| ATOM | 2374 | O | LEU | C3073 | 24.444 | −13.473 | −3.198 | 0.05 | 50.02 | O |
| ATOM | 2375 | N | GLY | C3074 | 22.616 | −12.492 | −4.076 | 1.00 | 49.38 | N |
| ATOM | 2376 | CA | GLY | C3074 | 21.779 | −13.663 | −3.899 | 1.00 | 48.02 | C |
| ATOM | 2377 | C | GLY | C3074 | 21.479 | −13.986 | −2.447 | 1.00 | 46.92 | C |
| ATOM | 2378 | O | GLY | C3074 | 22.391 | −14.150 | −1.642 | 1.00 | 46.73 | O |
| ATOM | 2379 | N | PHE | C3075 | 20.197 | −14.076 | −2.107 | 1.00 | 46.04 | N |
| ATOM | 2380 | CA | PHE | C3075 | 19.804 | −14.393 | −0.741 | 1.00 | 45.56 | C |
| ATOM | 2381 | CB | PHE | C3075 | 18.305 | −14.137 | −0.544 | 1.00 | 45.22 | C |
| ATOM | 2382 | CG | PHE | C3075 | 17.969 | −12.705 | −0.211 | 1.00 | 44.58 | C |
| ATOM | 2383 | CD1 | PHE | C3075 | 18.961 | −11.724 | −0.187 | 1.00 | 44.91 | C |
| ATOM | 2384 | CD2 | PHE | C3075 | 16.661 | −12.337 | 0.082 | 1.00 | 43.89 | C |
| ATOM | 2385 | CE1 | PHE | C3075 | 18.654 | −10.397 | 0.127 | 1.00 | 45.66 | C |
| ATOM | 2386 | CE2 | PHE | C3075 | 16.340 | −11.016 | 0.398 | 1.00 | 44.22 | C |
| ATOM | 2387 | CZ | PHE | C3075 | 17.338 | −10.042 | 0.420 | 1.00 | 44.74 | C |
| ATOM | 2388 | C | PHE | C3075 | 20.147 | −15.841 | −0.407 | 1.00 | 45.42 | C |
| ATOM | 2389 | O | PHE | C3075 | 20.099 | −16.251 | 0.754 | 1.00 | 44.99 | O |
| ATOM | 2390 | N | GLN | C3076 | 20.503 | −16.607 | −1.433 | 1.00 | 44.83 | N |
| ATOM | 2391 | CA | GLN | C3076 | 20.874 | −18.006 | −1.256 | 1.00 | 44.38 | C |
| ATOM | 2392 | CB | GLN | C3076 | 20.766 | −18.749 | −2.589 | 1.00 | 44.21 | C |
| ATOM | 2393 | CG | GLN | C3076 | 19.360 | −19.192 | −2.890 | 1.00 | 45.02 | C |
| ATOM | 2394 | CD | GLN | C3076 | 18.868 | −20.206 | −1.880 | 1.00 | 46.01 | C |
| ATOM | 2395 | OE1 | GLN | C3076 | 17.666 | −20.367 | −1.676 | 1.00 | 47.94 | O |
| ATOM | 2396 | NE2 | GLN | C3076 | 19.802 | −20.906 | −1.245 | 1.00 | 45.65 | N |
| ATOM | 2397 | C | GLN | C3076 | 22.287 | −18.133 | −0.706 | 1.00 | 43.75 | C |
| ATOM | 2398 | O | GLN | C3076 | 22.660 | −19.165 | −0.149 | 1.00 | 44.83 | O |
| ATOM | 2399 | N | ASP | C3077 | 23.070 | −17.074 | −0.862 | 1.00 | 43.01 | N |
| ATOM | 2400 | CA | ASP | C3077 | 24.442 | −17.065 | −0.377 | 1.00 | 41.11 | C |
| ATOM | 2401 | CB | ASP | C3077 | 25.315 | −16.254 | −1.333 | 1.00 | 44.24 | C |
| ATOM | 2402 | CG | ASP | C3077 | 25.261 | −16.789 | −2.756 | 1.00 | 45.88 | C |
| ATOM | 2403 | OD1 | ASP | C3077 | 25.692 | −17.943 | −2.974 | 1.00 | 47.04 | O |
| ATOM | 2404 | OD2 | ASP | C3077 | 24.777 | −16.064 | −3.652 | 1.00 | 46.86 | O |
| ATOM | 2405 | C | ASP | C3077 | 24.493 | −16.481 | 1.028 | 1.00 | 38.51 | C |
| ATOM | 2406 | O | ASP | C3077 | 25.564 | −16.291 | 1.598 | 1.00 | 39.35 | O |
| ATOM | 2407 | N | LEU | C3078 | 23.319 | −16.194 | 1.578 | 1.00 | 35.03 | N |
| ATOM | 2408 | CA | LEU | C3078 | 23.199 | −15.649 | 2.927 | 1.00 | 31.01 | C |
| ATOM | 2409 | CB | LEU | C3078 | 22.479 | −14.296 | 2.889 | 1.00 | 30.66 | C |
| ATOM | 2410 | CG | LEU | C3078 | 23.280 | −13.026 | 2.584 | 1.00 | 31.31 | C |
| ATOM | 2411 | CD1 | LEU | C3078 | 24.253 | −13.251 | 1.440 | 1.00 | 33.52 | C |
| ATOM | 2412 | CD2 | LEU | C3078 | 22.303 | −11.900 | 2.254 | 1.00 | 30.14 | C |
| ATOM | 2413 | C | LEU | C3078 | 22.396 | −16.632 | 3.778 | 1.00 | 28.65 | C |
| ATOM | 2414 | O | LEU | C3078 | 21.391 | −17.178 | 3.322 | 1.00 | 28.16 | O |
| ATOM | 2415 | N | GLU | C3079 | 22.839 | −16.860 | 5.009 | 1.00 | 25.38 | N |
| ATOM | 2416 | CA | GLU | C3079 | 22.138 | −17.779 | 5.895 | 1.00 | 24.35 | C |
| ATOM | 2417 | CB | GLU | C3079 | 22.794 | −19.163 | 5.869 | 1.00 | 24.49 | C |
| ATOM | 2418 | CG | GLU | C3079 | 21.999 | −20.224 | 6.616 | 1.00 | 24.73 | C |
| ATOM | 2419 | CD | GLU | C3079 | 22.653 | −21.592 | 6.558 | 1.00 | 26.90 | C |
| ATOM | 2420 | OE1 | GLU | C3079 | 23.770 | −21.740 | 7.089 | 1.00 | 28.68 | O |
| ATOM | 2421 | OE2 | GLU | C3079 | 22.053 | −22.523 | 5.979 | 1.00 | 29.27 | O |
| ATOM | 2422 | C | GLU | C3079 | 22.098 | −17.267 | 7.327 | 1.00 | 22.68 | C |
| ATOM | 2423 | O | GLU | C3079 | 23.123 | −16.873 | 7.883 | 1.00 | 22.88 | O |
| ATOM | 2424 | N | VAL | C3080 | 20.901 | −17.275 | 7.907 | 1.00 | 21.79 | N |
| ATOM | 2425 | CA | VAL | C3080 | 20.678 | −16.824 | 9.276 | 1.00 | 21.18 | C |
| ATOM | 2426 | CB | VAL | C3080 | 19.705 | −15.621 | 9.325 | 1.00 | 22.20 | C |
| ATOM | 2427 | CG1 | VAL | C3080 | 19.404 | −15.244 | 10.777 | 1.00 | 20.66 | C |
| ATOM | 2428 | CG2 | VAL | C3080 | 20.311 | −14.435 | 8.587 | 1.00 | 23.11 | C |
| ATOM | 2429 | C | VAL | C3080 | 20.064 | −17.971 | 10.060 | 1.00 | 20.45 | C |
| ATOM | 2430 | O | VAL | C3080 | 18.972 | −18.429 | 9.743 | 1.00 | 19.50 | O |
| ATOM | 2431 | N | LEU | C3081 | 20.781 | −18.441 | 11.072 | 1.00 | 20.00 | N |
| ATOM | 2432 | CA | LEU | C3081 | 20.303 | −19.540 | 11.897 | 1.00 | 21.15 | C |
| ATOM | 2433 | CB | LEU | C3081 | 21.328 | −20.686 | 11.905 | 1.00 | 21.34 | C |
| ATOM | 2434 | CG | LEU | C3081 | 21.677 | −21.275 | 10.528 | 1.00 | 22.27 | C |
| ATOM | 2435 | CD1 | LEU | C3081 | 22.713 | −22.396 | 10.653 | 1.00 | 23.04 | C |
| ATOM | 2436 | CD2 | LEU | C3081 | 20.400 | −21.802 | 9.884 | 1.00 | 24.47 | C |
| ATOM | 2437 | C | LEU | C3081 | 20.107 | −19.003 | 13.300 | 1.00 | 21.01 | C |
| ATOM | 2438 | O | LEU | C3081 | 20.287 | −17.811 | 13.541 | 1.00 | 20.54 | O |
| ATOM | 2439 | N | ASN | C3082 | 19.732 | −19.881 | 14.220 | 1.00 | 21.50 | N |
| ATOM | 2440 | CA | ASN | C3082 | 19.530 | −19.494 | 15.608 | 1.00 | 22.77 | C |
| ATOM | 2441 | CB | ASN | C3082 | 18.089 | −19.783 | 16.020 | 1.00 | 23.53 | C |
| ATOM | 2442 | CG | ASN | C3082 | 17.099 | −18.880 | 15.310 | 1.00 | 25.44 | C |
| ATOM | 2443 | OD1 | ASN | C3082 | 16.907 | −17.731 | 15.698 | 1.00 | 25.60 | O |
| ATOM | 2444 | ND2 | ASN | C3082 | 16.488 | −19.388 | 14.247 | 1.00 | 27.37 | N |
| ATOM | 2445 | C | ASN | C3082 | 20.499 | −20.258 | 16.499 | 1.00 | 22.68 | C |
| ATOM | 2446 | O | ASN | C3082 | 20.629 | −21.478 | 16.380 | 1.00 | 21.84 | O |
| ATOM | 2447 | N | ASN | C3083 | 21.191 | −19.541 | 17.382 | 1.00 | 23.99 | N |
| ATOM | 2448 | CA | ASN | C3083 | 22.137 | −20.187 | 18.282 | 1.00 | 23.34 | C |
| ATOM | 2449 | CB | ASN | C3083 | 23.141 | −19.170 | 18.840 | 1.00 | 24.83 | C |
| ATOM | 2450 | CG | ASN | C3083 | 22.474 | −18.019 | 19.575 | 1.00 | 25.31 | C |
| ATOM | 2451 | OD1 | ASN | C3083 | 21.405 | −18.175 | 20.174 | 1.00 | 23.71 | O |
| ATOM | 2452 | ND2 | ASN | C3083 | 23.116 | −16.859 | 19.550 | 1.00 | 26.83 | N |
| ATOM | 2453 | C | ASN | C3083 | 21.381 | −20.877 | 19.415 | 1.00 | 25.05 | C |
| ATOM | 2454 | O | ASN | C3083 | 20.152 | −20.876 | 19.432 | 1.00 | 22.22 | O |
| ATOM | 2455 | N | GLU | C3084 | 22.108 | −21.460 | 20.365 | 1.00 | 27.04 | N |
| ATOM | 2456 | CA | GLU | C3084 | 21.472 | −22.174 | 21.466 | 1.00 | 29.78 | C |
| ATOM | 2457 | CB | GLU | C3084 | 22.517 | −22.954 | 22.275 | 1.00 | 32.58 | C |
| ATOM | 2458 | CG | GLU | C3084 | 23.715 | −22.141 | 22.711 | 1.00 | 36.99 | C |
| ATOM | 2459 | CD | GLU | C3084 | 24.857 | −22.184 | 21.707 | 1.00 | 41.57 | C |
| ATOM | 2460 | OE1 | GLU | C3084 | 24.647 | −21.812 | 20.529 | 1.00 | 42.03 | O |
| ATOM | 2461 | OE2 | GLU | C3084 | 25.972 | −22.590 | 22.107 | 1.00 | 43.80 | O |
| ATOM | 2462 | C | GLU | C3084 | 20.638 | −21.294 | 22.396 | 1.00 | 30.13 | C |
| ATOM | 2463 | O | GLU | C3084 | 19.766 | −21.796 | 23.111 | 1.00 | 30.37 | O |
| ATOM | 2464 | N | ARG | C3085 | 20.901 | −19.991 | 22.401 | 1.00 | 28.98 | N |
| ATOM | 2465 | CA | ARG | C3085 | 20.119 | −19.088 | 23.238 | 1.00 | 30.28 | C |
| ATOM | 2466 | CB | ARG | C3085 | 20.976 | −17.912 | 23.716 | 1.00 | 31.17 | C |
| ATOM | 2467 | CG | ARG | C3085 | 22.055 | −18.340 | 24.705 | 1.00 | 34.80 | C |
| ATOM | 2468 | CD | ARG | C3085 | 23.056 | −17.237 | 24.995 | 1.00 | 37.35 | C |
| ATOM | 2469 | NE | ARG | C3085 | 24.367 | −17.801 | 25.310 | 1.00 | 40.35 | N |
| ATOM | 2470 | CZ | ARG | C3085 | 24.634 | −18.518 | 26.396 | 1.00 | 40.77 | C |
| ATOM | 2471 | NH1 | ARG | C3085 | 23.681 | −18.757 | 27.285 | 1.00 | 42.29 | N |
| ATOM | 2472 | NH2 | ARG | C3085 | 25.852 | −19.007 | 26.587 | 1.00 | 41.71 | N |
| ATOM | 2473 | C | ARG | C3085 | 18.918 | −18.597 | 22.438 | 1.00 | 29.32 | C |
| ATOM | 2474 | O | ARG | C3085 | 18.112 | −17.806 | 22.923 | 1.00 | 30.16 | O |
| ATOM | 2475 | N | GLY | C3086 | 18.805 | −19.086 | 21.207 | 1.00 | 27.92 | N |
| ATOM | 2476 | CA | GLY | C3086 | 17.690 | −18.714 | 20.350 | 1.00 | 26.11 | C |
| ATOM | 2477 | C | GLY | C3086 | 17.893 | −17.452 | 19.530 | 1.00 | 25.66 | C |
| ATOM | 2478 | O | GLY | C3086 | 17.005 | −17.044 | 18.782 | 1.00 | 26.33 | O |
| ATOM | 2479 | N | ALA | C3087 | 19.061 | −16.831 | 19.654 | 1.00 | 23.88 | N |
| ATOM | 2480 | CA | ALA | C3087 | 19.328 | −15.613 | 18.914 | 1.00 | 22.63 | C |
| ATOM | 2481 | CB | ALA | C3087 | 20.408 | −14.797 | 19.610 | 1.00 | 21.67 | C |
| ATOM | 2482 | C | ALA | C3087 | 19.742 | −15.893 | 17.478 | 1.00 | 22.49 | C |
| ATOM | 2483 | O | ALA | C3087 | 20.515 | −16.808 | 17.210 | 1.00 | 22.63 | O |
| ATOM | 2484 | N | PRO | C3088 | 19.220 | −15.098 | 16.532 | 1.00 | 21.73 | N |
| ATOM | 2485 | CD | PRO | 03088 | 18.185 | −14.068 | 16.740 | 1.00 | 21.19 | C |
| ATOM | 2486 | CA | PRO | C3088 | 19.536 | −15.244 | 15.112 | 1.00 | 20.08 | C |
| ATOM | 2487 | CB | PRO | C3088 | 18.434 | −14.435 | 14.435 | 1.00 | 22.01 | C |
| ATOM | 2488 | CG | PRO | C3088 | 18.196 | −13.325 | 15.412 | 1.00 | 23.33 | C |
| ATOM | 2489 | C | PRO | C3088 | 20.931 | −14.689 | 14.822 | 1.00 | 20.77 | C |
| ATOM | 2490 | O | PRO | C3088 | 21.360 | −13.704 | 15.434 | 1.00 | 19.53 | O |
| ATOM | 2491 | N | TYR | C3089 | 21.647 | −15.327 | 13.903 | 1.00 | 18.65 | N |
| ATOM | 2492 | CA | TYR | C3089 | 22.986 | −14.869 | 13.548 | 1.00 | 18.94 | C |
| ATOM | 2493 | CB | TYR | C3089 | 24.015 | −15.410 | 14.557 | 1.00 | 19.72 | C |
| ATOM | 2494 | CG | TYR | C3089 | 24.295 | −16.891 | 14.434 | 1.00 | 20.97 | C |
| ATOM | 2495 | CD1 | TYR | C3089 | 25.313 | −17.360 | 13.600 | 1.00 | 21.58 | C |
| ATOM | 2496 | CE1 | TYR | C3089 | 25.549 | −18.721 | 13.448 | 1.00 | 21.20 | C |
| ATOM | 2497 | CD2 | TYR | C3089 | 23.521 | −17.826 | 15.118 | 1.00 | 21.44 | C |
| ATOM | 2498 | CE2 | TYR | C3089 | 23.748 | −19.193 | 14.974 | 1.00 | 21.49 | C |
| ATOM | 2499 | CZ | TYR | C3089 | 24.764 | −19.630 | 14.133 | 1.00 | 20.90 | C |
| ATOM | 2500 | OH | TYR | C3089 | 24.980 | −20.972 | 13.961 | 1.00 | 21.13 | O |
| ATOM | 2501 | C | TYR | C3089 | 23.310 | −15.356 | 12.142 | 1.00 | 19.33 | C |
| ATOM | 2502 | O | TYR | C3089 | 22.695 | −16.305 | 11.658 | 1.00 | 18.04 | O |
| ATOM | 2503 | N | PHE | C3090 | 24.252 | −14.694 | 11.476 | 1.00 | 21.68 | N |
| ATOM | 2504 | CA | PHE | C3090 | 24.644 | −15.095 | 10.129 | 1.00 | 22.43 | C |
| ATOM | 2505 | CB | PHE | C3090 | 25.224 | −13.921 | 9.343 | 1.00 | 21.81 | C |
| ATOM | 2506 | CG | PHE | C3090 | 24.187 | −13.057 | 8.686 | 1.00 | 22.97 | C |
| ATOM | 2507 | CD1 | PHE | C3090 | 23.627 | −11.980 | 9.364 | 1.00 | 22.58 | C |
| ATOM | 2508 | CD2 | PHE | C3090 | 23.774 | −13.317 | 7.382 | 1.00 | 22.20 | C |
| ATOM | 2509 | CE1 | PHE | C3090 | 22.670 | −11.172 | 8.752 | 1.00 | 21.60 | C |
| ATOM | 2510 | CE2 | PHE | C3090 | 22.815 | −12.512 | 6.762 | 1.00 | 21.04 | C |
| ATOM | 2511 | CZ | PHE | C3090 | 22.267 | −11.439 | 7.453 | 1.00 | 20.05 | C |
| ATOM | 2512 | C | PHE | C3090 | 25.681 | −16.201 | 10.185 | 1.00 | 23.74 | C |
| ATOM | 2513 | O | PHE | C3090 | 26.804 | −15.992 | 10.642 | 1.00 | 23.80 | O |
| ATOM | 2514 | N | SER | C3091 | 25.291 | −17.381 | 9.727 | 1.00 | 25.13 | N |
| ATOM | 2515 | CA | SER | C3091 | 26.186 | −18.522 | 9.707 | 1.00 | 24.76 | C |
| ATOM | 2516 | CB | SER | C3091 | 25.378 | −19.805 | 9.790 | 1.00 | 25.31 | C |
| ATOM | 2517 | OG | SER | C3091 | 24.465 | −19.856 | 8.709 | 1.00 | 25.46 | O |
| ATOM | 2518 | C | SER | C3091 | 26.959 | −18.493 | 8.393 | 1.00 | 25.15 | C |
| ATOM | 2519 | O | SER | C3091 | 28.036 | −19.075 | 8.285 | 1.00 | 24.95 | O |
| ATOM | 2520 | N | GLN | C3092 | 26.398 | −17.806 | 7.401 | 1.00 | 25.28 | N |
| ATOM | 2521 | CA | GLN | C3092 | 27.012 | −17.701 | 6.079 | 1.00 | 26.50 | C |
| ATOM | 2522 | CB | GLN | C3092 | 26.414 | −18.759 | 5.150 | 1.00 | 29.33 | C |
| ATOM | 2523 | CG | GLN | C3092 | 26.948 | −18.723 | 3.728 | 1.00 | 33.15 | C |
| ATOM | 2524 | CD | GLN | C3092 | 28.353 | −19.271 | 3.638 | 1.00 | 35.44 | C |
| ATOM | 2525 | OE1 | GLN | C3092 | 29.292 | −18.711 | 4.204 | 1.00 | 36.58 | O |
| ATOM | 2526 | NE2 | GLN | C3092 | 28.503 | −20.384 | 2.935 | 1.00 | 37.04 | N |
| ATOM | 2527 | C | GLN | C3092 | 26.780 | −16.323 | 5.465 | 1.00 | 25.49 | C |
| ATOM | 2528 | O | GLN | C3092 | 25.637 | −15.871 | 5.365 | 1.00 | 24.76 | O |
| ATOM | 2529 | N | ALA | C3093 | 27.864 | −15.674 | 5.042 | 1.00 | 24.56 | N |
| ATOM | 2530 | CA | ALA | C3093 | 27.801 | −14.350 | 4.422 | 1.00 | 24.82 | C |
| ATOM | 2531 | CB | ALA | C3093 | 27.400 | −13.302 | 5.451 | 1.00 | 24.36 | C |
| ATOM | 2532 | C | ALA | C3093 | 29.152 | −13.978 | 3.808 | 1.00 | 24.47 | C |
| ATOM | 2533 | O | ALA | C3093 | 30.200 | −14.284 | 4.365 | 1.00 | 24.58 | O |
| ATOM | 2534 | N | PRO | C3094 | 29.137 | −13.289 | 2.654 | 1.00 | 25.93 | N |
| ATOM | 2535 | CD | PRO | C3094 | 27.920 | −12.815 | 1.965 | 1.00 | 24.23 | C |
| ATOM | 2536 | CA | PRO | C3094 | 30.345 | −12.860 | 1.935 | 1.00 | 25.56 | C |
| ATOM | 2537 | CB | PRO | C3094 | 29.788 | −12.404 | 0.592 | 1.00 | 26.20 | C |
| ATOM | 2538 | CG | PRO | C3094 | 28.472 | −11.792 | 0.997 | 1.00 | 26.19 | C |
| ATOM | 2539 | C | PRO | C3094 | 31.098 | −11.744 | 2.658 | 1.00 | 26.12 | C |
| ATOM | 2540 | O | PRO | C3094 | 31.403 | −10.703 | 2.077 | 1.00 | 25.63 | O |
| ATOM | 2541 | N | PHE | C3095 | 31.404 | −11.971 | 3.930 | 1.00 | 26.90 | N |
| ATOM | 2542 | CA | PHE | C3095 | 32.098 | −10.977 | 4.739 | 1.00 | 26.88 | C |
| ATOM | 2543 | CB | PHE | C3095 | 31.068 | −10.004 | 5.331 | 1.00 | 26.61 | C |
| ATOM | 2544 | CG | PHE | C3095 | 31.665 | −8.923 | 6.179 | 1.00 | 26.50 | C |
| ATOM | 2545 | CD1 | PHE | C3095 | 31.491 | −8.931 | 7.559 | 1.00 | 25.34 | C |
| ATOM | 2546 | CD2 | PHE | C3095 | 32.406 | −7.895 | 5.601 | 1.00 | 26.22 | C |
| ATOM | 2547 | CE1 | PHE | C3095 | 32.043 | −7.937 | 8.350 | 1.00 | 24.07 | C |
| ATOM | 2548 | CE2 | PHE | C3095 | 32.964 | −6.895 | 6.384 | 1.00 | 25.18 | C |
| ATOM | 2549 | CZ | PHE | C3095 | 32.781 | −6.917 | 7.764 | 1.00 | 26.71 | C |
| ATOM | 2550 | C | PHE | C3095 | 32.830 | −11.726 | 5.841 | 1.00 | 27.07 | C |
| ATOM | 2551 | O | PHE | C3095 | 32.234 | −12.547 | 6.527 | 1.00 | 28.82 | O |
| ATOM | 2552 | N | SER | C3096 | 34.119 | −11.450 | 6.013 | 1.00 | 27.08 | N |
| ATOM | 2553 | CA | SER | C3096 | 34.907 | −12.153 | 7.022 | 1.00 | 26.87 | C |
| ATOM | 2554 | CB | SER | C3096 | 36.287 | −12.488 | 6.457 | 1.00 | 27.85 | C |
| ATOM | 2555 | OG | SER | C3096 | 36.967 | −11.315 | 6.073 | 1.00 | 29.01 | O |
| ATOM | 2556 | C | SER | C3096 | 35.072 | −11.446 | 8.364 | 1.00 | 27.60 | C |
| ATOM | 2557 | O | SER | C3096 | 35.753 | −11.957 | 9.251 | 1.00 | 28.27 | O |
| ATOM | 2558 | N | GLY | C3097 | 34.466 | −10.271 | 8.513 | 1.00 | 26.02 | N |
| ATOM | 2559 | CA | GLY | C3097 | 34.559 | −9.558 | 9.775 | 1.00 | 25.74 | C |
| ATOM | 2560 | C | GLY | C3097 | 33.406 | −9.991 | 10.664 | 1.00 | 26.75 | C |
| ATOM | 2561 | O | GLY | C3097 | 32.796 | −11.029 | 10.422 | 1.00 | 26.07 | O |
| ATOM | 2562 | N | LYS | C3098 | 33.084 | −9.202 | 11.682 | 1.00 | 25.99 | N |
| ATOM | 2563 | CA | LYS | C3098 | 31.985 | −9.559 | 12.572 | 1.00 | 26.80 | C |
| ATOM | 2564 | CB | LYS | C3098 | 32.302 | −9.106 | 13.998 | 1.00 | 28.83 | C |
| ATOM | 2565 | CG | LYS | C3098 | 33.742 | −9.410 | 14.383 | 1.00 | 33.82 | C |
| ATOM | 2566 | CD | LYS | C3098 | 34.014 | −9.227 | 15.860 | 1.00 | 37.34 | C |
| ATOM | 2567 | CE | LYS | C3098 | 33.574 | −10.444 | 16.652 | 1.00 | 39.78 | C |
| ATOM | 2568 | NZ | LYS | C3098 | 33.986 | −10.326 | 18.084 | 1.00 | 40.81 | N |
| ATOM | 2569 | C | LYS | C3098 | 30.676 | −8.944 | 12.095 | 1.00 | 25.06 | C |
| ATOM | 2570 | O | LYS | C3098 | 30.633 | −7.785 | 11.670 | 1.00 | 24.85 | O |
| ATOM | 2571 | N | ILE | C3099 | 29.611 | −9.737 | 12.152 | 1.00 | 22.88 | N |
| ATOM | 2572 | CA | ILE | C3099 | 28.292 | −9.286 | 11.730 | 1.00 | 20.01 | C |
| ATOM | 2573 | CB | ILE | C3099 | 27.688 | −10.246 | 10.687 | 1.00 | 19.89 | C |
| ATOM | 2574 | CG2 | ILE | C3099 | 26.373 | −9.674 | 10.150 | 1.00 | 15.87 | C |
| ATOM | 2575 | CG1 | ILE | C3099 | 28.686 | −10.474 | 9.548 | 1.00 | 18.98 | C |
| ATOM | 2576 | CD1 | ILE | C3099 | 28.241 | −11.543 | 8.561 | 1.00 | 19.71 | C |
| ATOM | 2577 | C | ILE | C3099 | 27.364 | −9.243 | 12.941 | 1.00 | 20.05 | C |
| ATOM | 2578 | O | ILE | C3099 | 26.967 | −10.289 | 13.459 | 1.00 | 21.97 | O |
| ATOM | 2579 | N | TRP | C3100 | 27.032 | −8.038 | 13.398 | 1.00 | 18.21 | N |
| ATOM | 2580 | CA | TRP | C3100 | 26.140 | −7.880 | 14.541 | 1.00 | 17.20 | C |
| ATOM | 2581 | CB | TRP | C3100 | 26.570 | −6.678 | 15.383 | 1.00 | 17.48 | C |
| ATOM | 2582 | CG | TRP | C3100 | 27.959 | −6.851 | 15.948 | 1.00 | 17.00 | C |
| ATOM | 2583 | CD2 | TRP | C3100 | 28.323 | −7.642 | 17.085 | 1.00 | 16.80 | C |
| ATOM | 2584 | CE2 | TRP | C3100 | 29.724 | −7.538 | 17.237 | 1.00 | 18.05 | C |
| ATOM | 2585 | CE3 | TRP | C3100 | 27.599 | −8.431 | 17.992 | 1.00 | 19.31 | C |
| ATOM | 2586 | CD1 | TRP | C3100 | 29.119 | −6.316 | 15.465 | 1.00 | 17.86 | C |
| ATOM | 2587 | NE1 | TRP | C3100 | 30.188 | −6.724 | 16.236 | 1.00 | 18.87 | N |
| ATOM | 2588 | CZ2 | TRP | C3100 | 30.417 | −8.192 | 18.263 | 1.00 | 19.19 | C |
| ATOM | 2589 | CZ3 | TRP | C3100 | 28.290 | −9.082 | 19.012 | 1.00 | 19.20 | C |
| ATOM | 2590 | CH2 | TRP | C3100 | 29.687 | −8.955 | 19.136 | 1.00 | 17.94 | C |
| ATOM | 2591 | C | TRP | C3100 | 24.717 | −7.699 | 14.018 | 1.00 | 17.56 | C |
| ATOM | 2592 | O | TRP | C3100 | 24.365 | −6.656 | 13.461 | 1.00 | 18.14 | O |
| ATOM | 2593 | N | LEU | C3101 | 23.916 | −8.742 | 14.191 | 1.00 | 17.00 | N |
| ATOM | 2594 | CA | LEU | C3101 | 22.540 | −8.764 | 13.725 | 1.00 | 16.11 | C |
| ATOM | 2595 | CB | LEU | C3101 | 22.321 | −9.991 | 12.837 | 1.00 | 16.56 | C |
| ATOM | 2596 | CG | LEU | C3101 | 20.875 | −10.417 | 12.559 | 1.00 | 16.77 | C |
| ATOM | 2597 | CD1 | LEU | C3101 | 20.194 | −9.413 | 11.625 | 1.00 | 15.60 | C |
| ATOM | 2598 | CD2 | LEU | C3101 | 20.883 | −11.809 | 11.939 | 1.00 | 18.41 | C |
| ATOM | 2599 | C | LEU | C3101 | 21.525 | −8.811 | 14.852 | 1.00 | 16.81 | C |
| ATOM | 2600 | O | LEU | C3101 | 21.746 | −9.435 | 15.890 | 1.00 | 14.02 | O |
| ATOM | 2601 | N | SER | C3102 | 20.410 | −8.128 | 14.638 | 1.00 | 15.53 | N |
| ATOM | 2602 | CA | SER | C3102 | 19.316 | −8.151 | 15.588 | 1.00 | 14.91 | C |
| ATOM | 2603 | CB | SER | C3102 | 19.424 | −7.035 | 16.619 | 1.00 | 14.40 | C |
| ATOM | 2604 | OG | SER | C3102 | 18.483 | −7.263 | 17.657 | 1.00 | 15.41 | O |
| ATOM | 2605 | C | SER | C3102 | 18.052 | −7.981 | 14.761 | 1.00 | 14.92 | C |
| ATOM | 2606 | O | SER | C3102 | 18.020 | −7.195 | 13.805 | 1.00 | 14.51 | O |
| ATOM | 2607 | N | ILE | C3103 | 17.023 | −8.734 | 15.129 | 1.00 | 14.41 | N |
| ATOM | 2608 | CA | ILE | C3103 | 15.744 | −8.710 | 14.437 | 1.00 | 15.86 | C |
| ATOM | 2609 | CB | ILE | C3103 | 15.490 | −10.043 | 13.668 | 1.00 | 16.91 | C |
| ATOM | 2610 | CG2 | ILE | C3103 | 14.171 | −9.958 | 12.880 | 1.00 | 18.33 | C |
| ATOM | 2611 | CG1 | ILE | C3103 | 16.665 | −10.353 | 12.737 | 1.00 | 18.54 | C |
| ATOM | 2612 | CD1 | ILE | C3103 | 16.557 | −11.729 | 12.042 | 1.00 | 20.00 | C |
| ATOM | 2613 | C | ILE | C3103 | 14.657 | −8.608 | 15.495 | 1.00 | 17.43 | C |
| ATOM | 2614 | O | ILE | C3103 | 14.805 | −9.148 | 16.594 | 1.00 | 17.45 | O |
| ATOM | 2615 | N | SER | C3104 | 13.566 | −7.929 | 15.157 | 1.00 | 18.17 | N |
| ATOM | 2616 | CA | SER | C3104 | 12.439 | −7.798 | 16.070 | 1.00 | 20.57 | C |
| ATOM | 2617 | CB | SER | C3104 | 12.610 | −6.583 | 16.986 | 1.00 | 19.79 | C |
| ATOM | 2618 | OG | SER | C3104 | 11.596 | −6.571 | 17.981 | 1.00 | 23.17 | O |
| ATOM | 2619 | C | SER | C3104 | 11.188 | −7.645 | 15.214 | 1.00 | 22.21 | C |
| ATOM | 2620 | O | SER | C3104 | 11.269 | −7.301 | 14.033 | 1.00 | 22.47 | O |
| ATOM | 2621 | N | HIS | C3105 | 10.028 | −7.889 | 15.796 | 1.00 | 23.30 | N |
| ATOM | 2622 | CA | HIS | C3105 | 8.818 | −7.784 | 15.009 | 1.00 | 25.44 | C |
| ATOM | 2623 | CB | HIS | C3105 | 8.597 | −9.083 | 14.241 | 1.00 | 27.72 | C |
| ATOM | 2624 | CG | HIS | C3105 | 8.509 | −10.287 | 15.125 | 1.00 | 29.50 | C |
| ATOM | 2625 | CD2 | HIS | C3105 | 7.438 | −10.938 | 15.639 | 1.00 | 33.02 | C |
| ATOM | 2626 | ND1 | HIS | C3105 | 9.622 | −10.934 | 15.615 | 1.00 | 32.99 | N |
| ATOM | 2627 | CE1 | HIS | C3105 | 9.242 | −11.933 | 16.393 | 1.00 | 34.27 | C |
| ATOM | 2628 | NE2 | HIS | C3105 | 7.922 | −11.956 | 16.424 | 1.00 | 33.98 | N |
| ATOM | 2629 | C | HIS | C3105 | 7.565 | −7.489 | 15.805 | 1.00 | 26.04 | C |
| ATOM | 2630 | O | HIS | C3105 | 7.499 | −7.729 | 17.009 | 1.00 | 23.14 | O |
| ATOM | 2631 | N | THR | C3106 | 6.575 | −6.972 | 15.086 | 1.00 | 28.54 | N |
| ATOM | 2632 | CA | THR | C3106 | 5.261 | −6.652 | 15.613 | 1.00 | 30.04 | C |
| ATOM | 2633 | CB | THR | C3106 | 4.855 | −5.209 | 15.300 | 1.00 | 31.60 | C |
| ATOM | 2634 | OG1 | THR | C3106 | 4.804 | −5.030 | 13.878 | 1.00 | 31.75 | O |
| ATOM | 2635 | CG2 | THR | C3106 | 5.848 | −4.231 | 15.905 | 1.00 | 30.63 | C |
| ATOM | 2636 | C | THR | C3106 | 4.332 | −7.576 | 14.840 | 1.00 | 30.05 | C |
| ATOM | 2637 | O | THR | C3106 | 4.792 | −8.451 | 14.101 | 1.00 | 31.00 | O |
| ATOM | 2638 | N | ASP | C3107 | 3.031 | −7.374 | 14.987 | 1.00 | 31.29 | N |
| ATOM | 2639 | CA | ASP | C3107 | 2.056 | −8.206 | 14.296 | 1.00 | 32.30 | C |
| ATOM | 2640 | CB | ASP | C3107 | 0.674 | −8.033 | 14.934 | 1.00 | 36.82 | C |
| ATOM | 2641 | CG | ASP | C3107 | 0.596 | −8.612 | 16.327 | 1.00 | 40.78 | C |
| ATOM | 2642 | OD1 | ASP | C3107 | −0.205 | −8.097 | 17.136 | 1.00 | 44.76 | O |
| ATOM | 2643 | OD2 | ASP | C3107 | 1.319 | −9.591 | 16.612 | 1.00 | 44.36 | O |
| ATOM | 2644 | C | ASP | C3107 | 1.959 | −7.890 | 12.807 | 1.00 | 31.24 | C |
| ATOM | 2645 | O | ASP | C3107 | 1.521 | −8.729 | 12.026 | 1.00 | 32.84 | O |
| ATOM | 2646 | N | GLN | C3108 | 2.374 | −6.690 | 12.410 | 1.00 | 28.20 | N |
| ATOM | 2647 | CA | GLN | C3108 | 2.284 | −6.289 | 11.009 | 1.00 | 27.03 | C |
| ATOM | 2648 | CB | GLN | C3108 | 1.590 | −4.928 | 10.877 | 0.05 | 26.57 | C |
| ATOM | 2649 | CG | GLN | C3108 | 0.284 | −4.788 | 11.626 | 0.05 | 26.25 | C |
| ATOM | 2650 | CD | GLN | C3108 | 0.486 | −4.733 | 13.121 | 0.05 | 26.05 | C |
| ATOM | 2651 | OE1 | GLN | C3108 | 1.220 | −3.887 | 13.631 | 0.05 | 25.96 | O |
| ATOM | 2652 | NE2 | GLN | C3108 | −0.165 | −5.636 | 13.834 | 0.05 | 25.84 | N |
| ATOM | 2653 | C | GLN | C3108 | 3.607 | −6.204 | 10.264 | 1.00 | 25.37 | C |
| ATOM | 2654 | O | GLN | C3108 | 3.624 | −6.210 | 9.032 | 1.00 | 24.85 | O |
| ATOM | 2655 | N | PHE | C3109 | 4.716 | −6.098 | 10.987 | 1.00 | 24.20 | N |
| ATOM | 2656 | CA | PHE | C3109 | 5.996 | −5.994 | 10.299 | 1.00 | 23.19 | C |
| ATOM | 2657 | CB | PHE | C3109 | 6.210 | −4.548 | 9.827 | 1.00 | 24.65 | C |
| ATOM | 2658 | CG | PHE | C3109 | 6.268 | −3.541 | 10.947 | 1.00 | 26.19 | C |
| ATOM | 2659 | CD1 | PHE | C3109 | 7.406 | −3.421 | 11.738 | 1.00 | 26.36 | C |
| ATOM | 2660 | CD2 | PHE | C3109 | 5.181 | −2.714 | 11.211 | 1.00 | 27.63 | C |
| ATOM | 2661 | CE1 | PHE | C3109 | 7.464 | −2.490 | 12.780 | 1.00 | 26.39 | C |
| ATOM | 2662 | CE2 | PHE | C3109 | 5.225 | −1.780 | 12.249 | 1.00 | 28.63 | C |
| ATOM | 2663 | CZ | PHE | C3109 | 6.371 | −1.669 | 13.036 | 1.00 | 28.60 | C |
| ATOM | 2664 | C | PHE | C3109 | 7.191 | −6.459 | 11.116 | 1.00 | 21.14 | C |
| ATOM | 2665 | O | PHE | C3109 | 7.100 | −6.648 | 12.328 | 1.00 | 19.22 | O |
| ATOM | 2666 | N | VAL | C3110 | 8.308 | −6.650 | 10.425 | 1.00 | 19.26 | N |
| ATOM | 2667 | CA | VAL | C3110 | 9.550 | −7.083 | 11.052 | 1.00 | 19.85 | C |
| ATOM | 2668 | CB | VAL | C3110 | 10.027 | −8.438 | 10.471 | 1.00 | 21.25 | C |
| ATOM | 2669 | CG1 | VAL | C3110 | 11.360 | −8.859 | 11.101 | 1.00 | 23.25 | C |
| ATOM | 2670 | CG2 | VAL | C3110 | 8.980 | −9.488 | 10.719 | 1.00 | 25.17 | C |
| ATOM | 2671 | C | VAL | C3110 | 10.604 | −6.039 | 10.737 | 1.00 | 19.21 | C |
| ATOM | 2672 | O | VAL | C3110 | 10.503 | −5.345 | 9.725 | 1.00 | 19.53 | O |
| ATOM | 2673 | N | THR | C3111 | 11.603 | −5.925 | 11.608 | 1.00 | 19.09 | N |
| ATOM | 2674 | CA | THR | C3111 | 12.695 | −4.984 | 11.409 | 1.00 | 20.72 | C |
| ATOM | 2675 | CB | THR | C3111 | 12.608 | −3.765 | 12.368 | 1.00 | 22.75 | C |
| ATOM | 2676 | OG1 | THR | C3111 | 12.635 | −4.216 | 13.730 | 1.00 | 26.03 | O |
| ATOM | 2677 | CG2 | THR | C3111 | 11.322 | −2.976 | 12.124 | 1.00 | 22.96 | C |
| ATOM | 2678 | C | THR | C3111 | 13.993 | −5.725 | 11.684 | 1.00 | 20.16 | C |
| ATOM | 2679 | O | THR | C3111 | 14.024 | −6.664 | 12.481 | 1.00 | 20.27 | O |
| ATOM | 2680 | N | ALA | C3112 | 15.060 | −5.312 | 11.010 | 1.00 | 19.72 | N |
| ATOM | 2681 | CA | ALA | C3112 | 16.361 | −5.935 | 11.201 | 1.00 | 19.77 | C |
| ATOM | 2682 | CB | ALA | C3112 | 16.617 | −6.975 | 10.109 | 1.00 | 18.61 | C |
| ATOM | 2683 | C | ALA | C3112 | 17.441 | −4.868 | 11.172 | 1.00 | 19.41 | C |
| ATOM | 2684 | O | ALA | C3112 | 17.337 | −3.885 | 10.442 | 1.00 | 22.32 | O |
| ATOM | 2685 | N | SER | C3113 | 18.474 | −5.062 | 11.980 | 1.00 | 20.21 | N |
| ATOM | 2686 | CA | SER | C3113 | 19.579 | −4.124 | 12.045 | 1.00 | 18.53 | C |
| ATOM | 2687 | CB | SER | C3113 | 19.506 | −3.298 | 13.327 | 1.00 | 19.50 | C |
| ATOM | 2688 | OG | SER | C3113 | 20.510 | −2.293 | 13.328 | 1.00 | 19.90 | O |
| ATOM | 2689 | C | SER | C3113 | 20.888 | −4.895 | 12.003 | 1.00 | 18.71 | C |
| ATOM | 2690 | O | SER | C3113 | 21.084 | −5.843 | 12.767 | 1.00 | 16.55 | O |
| ATOM | 2691 | N | VAL | C3114 | 21.779 | −4.472 | 11.107 | 1.00 | 17.09 | N |
| ATOM | 2692 | CA | VAL | C3114 | 23.071 | −5.115 | 10.930 | 1.00 | 17.37 | C |
| ATOM | 2693 | CB | VAL | C3114 | 23.145 | −5.842 | 9.563 | 1.00 | 16.61 | C |
| ATOM | 2694 | CG1 | VAL | C3114 | 24.593 | −6.263 | 9.255 | 1.00 | 18.34 | C |
| ATOM | 2695 | CG2 | VAL | C3114 | 22.244 | −7.050 | 9.576 | 1.00 | 16.94 | C |
| ATOM | 2696 | C | VAL | C3114 | 24.216 | −4.116 | 10.993 | 1.00 | 16.94 | C |
| ATOM | 2697 | O | VAL | C3114 | 24.145 | −3.043 | 10.408 | 1.00 | 16.28 | O |
| ATOM | 2698 | N | ILE | C3115 | 25.265 | −4.477 | 11.720 | 1.00 | 17.86 | N |
| ATOM | 2699 | CA | ILE | C3115 | 26.446 | −3.632 | 11.820 | 1.00 | 19.21 | C |
| ATOM | 2700 | CB | ILE | C3115 | 26.667 | −3.085 | 13.253 | 1.00 | 19.71 | C |
| ATOM | 2701 | CG2 | ILE | C3115 | 27.926 | −2.217 | 13.287 | 1.00 | 19.20 | C |
| ATOM | 2702 | CG1 | ILE | C3115 | 25.451 | −2.268 | 13.703 | 1.00 | 19.12 | C |
| ATOM | 2703 | CD1 | ILE | C3115 | 25.573 | −1.701 | 15.110 | 1.00 | 20.38 | C |
| ATOM | 2704 | C | ILE | C3115 | 27.627 | −4.516 | 11.440 | 1.00 | 18.95 | C |
| ATOM | 2705 | O | ILE | C3115 | 27.806 | −5.589 | 12.007 | 1.00 | 18.31 | O |
| ATOM | 2706 | N | LEU | C3116 | 28.409 | −4.076 | 10.457 | 1.00 | 19.22 | N |
| ATOM | 2707 | CA | LEU | C3116 | 29.578 | −4.830 | 10.021 | 1.00 | 20.77 | C |
| ATOM | 2708 | CB | LEU | C3116 | 29.696 | −4.805 | 8.493 | 1.00 | 20.70 | C |
| ATOM | 2709 | CG | LEU | C3116 | 28.445 | −5.235 | 7.703 | 1.00 | 19.25 | C |
| ATOM | 2710 | CD1 | LEU | C3116 | 28.758 | −5.249 | 6.218 | 1.00 | 21.02 | C |
| ATOM | 2711 | CD2 | LEU | C3116 | 27.996 | −6.614 | 8.150 | 1.00 | 19.48 | C |
| ATOM | 2712 | C | LEU | C3116 | 30.803 | −4.182 | 10.655 | 1.00 | 21.44 | C |
| ATOM | 2713 | O | LEU | C3116 | 30.996 | −2.969 | 10.554 | 1.00 | 21.12 | O |
| ATOM | 2714 | N | GLU | C3117 | 31.620 | −4.994 | 11.314 | 1.00 | 23.48 | N |
| ATOM | 2715 | CA | GLU | C3117 | 32.812 | −4.493 | 11.987 | 1.00 | 26.83 | C |
| ATOM | 2716 | CB | GLU | C3117 | 32.571 | −4.457 | 13.504 | 1.00 | 27.48 | C |
| ATOM | 2717 | CG | GLU | C3117 | 33.817 | −4.166 | 14.343 | 1.00 | 30.16 | C |
| ATOM | 2718 | CD | GLU | C3117 | 33.628 | −4.470 | 15.826 | 1.00 | 29.66 | C |
| ATOM | 2719 | OE1 | GLU | C3117 | 33.182 | −5.595 | 16.158 | 1.00 | 29.18 | O |
| ATOM | 2720 | OE2 | GLU | C3117 | 33.932 | −3.585 | 16.660 | 1.00 | 29.22 | O |
| ATOM | 2721 | C | GLU | C3117 | 34.048 | −5.335 | 11.680 | 1.00 | 28.97 | C |
| ATOM | 2722 | O | GLU | C3117 | 33.978 | −6.563 | 11.578 | 1.00 | 27.81 | O |
| ATOM | 2723 | N | GLU | C3118 | 35.178 | −4.657 | 11.513 | 1.00 | 31.18 | N |
| ATOM | 2724 | CA | GLU | C3118 | 36.446 | −5.323 | 11.248 | 1.00 | 35.42 | C |
| ATOM | 2725 | CB | GLU | C3118 | 37.015 | −4.880 | 9.893 | 1.00 | 37.41 | C |
| ATOM | 2726 | CG | GLU | C3118 | 36.592 | −5.772 | 8.727 | 1.00 | 41.62 | C |
| ATOM | 2727 | CD | GLU | C3118 | 37.041 | −5.248 | 7.368 | 1.00 | 43.91 | C |
| ATOM | 2728 | OE1 | GLU | C3118 | 38.193 | −4.776 | 7.253 | 1.00 | 46.07 | O |
| ATOM | 2729 | OE2 | GLU | C3118 | 36.242 | −5.321 | 6.409 | 1.00 | 44.10 | O |
| ATOM | 2730 | C | GLU | C3118 | 37.414 | −4.963 | 12.369 | 1.00 | 35.74 | C |
| ATOM | 2731 | O | GLU | C3118 | 37.417 | −3.784 | 12.785 | 1.00 | 35.88 | O |
| ATOM | 2732 | OXT | GLU | C3118 | 38.154 | −5.858 | 12.821 | 1.00 | 38.35 | O |
| TER | 2733 | | GLU | C3118 | | | | | | |
| ATOM | 2734 | O | HOH | W4002 | 24.915 | 17.154 | 36.149 | 1.00 | 24.34 | O |
| ATOM | 2735 | O | HOH | W4003 | 1.430 | 5.412 | 7.521 | 1.00 | 22.65 | O |
| ATOM | 2736 | O | HOH | W4004 | 15.230 | −11.883 | 17.512 | 1.00 | 29.62 | O |
| ATOM | 2737 | O | HOH | W4006 | 20.551 | 4.946 | 39.709 | 1.00 | 33.21 | O |
| ATOM | 2738 | O | HOH | W4008 | 0.497 | 12.883 | 37.515 | 1.00 | 46.32 | O |
| ATOM | 2739 | O | HOH | W4009 | 22.219 | 1.070 | 4.722 | 1.00 | 20.90 | O |
| ATOM | 2740 | O | HOH | W4010 | 23.057 | 4.216 | 27.783 | 1.00 | 22.10 | O |
| ATOM | 2741 | O | HOH | W4012 | 26.158 | 9.664 | 6.047 | 1.00 | 26.69 | O |
| ATOM | 2742 | O | HOH | W4013 | 13.195 | −14.652 | 11.137 | 1.00 | 18.24 | O |
| ATOM | 2743 | O | HOH | W4014 | 32.126 | −19.177 | 9.562 | 1.00 | 41.78 | O |
| ATOM | 2744 | O | HOH | W4016 | 11.365 | 8.055 | −2.295 | 1.00 | 29.34 | O |
| ATOM | 2745 | O | HOH | W4017 | 16.979 | 7.951 | 22.041 | 1.00 | 23.20 | O |
| ATOM | 2746 | O | HOH | W4018 | 20.734 | 17.150 | 36.413 | 1.00 | 25.86 | O |
| ATOM | 2747 | O | HOH | W4020 | −9.281 | −6.387 | 41.928 | 1.00 | 31.95 | O |
| ATOM | 2748 | O | HOH | W4021 | 18.869 | −12.662 | 22.632 | 1.00 | 30.51 | O |
| ATOM | 2749 | O | HOH | W4025 | 18.802 | 13.286 | −2.320 | 1.00 | 31.86 | O |
| ATOM | 2750 | O | HOH | W4026 | 26.500 | 16.583 | 31.811 | 1.00 | 52.21 | O |
| ATOM | 2751 | O | HOH | W4027 | 6.858 | −4.124 | 3.265 | 1.00 | 23.26 | O |
| ATOM | 2752 | O | HOH | W4028 | 10.702 | 1.088 | −3.405 | 1.00 | 41.81 | O |
| ATOM | 2753 | O | HOH | W4029 | 12.811 | 8.598 | 27.541 | 1.00 | 33.12 | O |
| ATOM | 2754 | O | HOH | W4031 | 12.749 | −8.445 | 19.973 | 1.00 | 24.85 | O |
| ATOM | 2755 | O | HOH | W4032 | 13.535 | 0.610 | 2.032 | 1.00 | 40.39 | O |
| ATOM | 2756 | O | HOH | W4033 | 13.594 | 8.162 | 33.675 | 1.00 | 30.23 | O |
| ATOM | 2757 | O | HOH | W4035 | 10.527 | 5.028 | 27.661 | 1.00 | 22.20 | O |
| ATOM | 2758 | O | HOH | W4038 | 10.071 | 6.775 | 24.507 | 1.00 | 34.64 | O |
| ATOM | 2759 | O | HOH | W4039 | 22.581 | 2.348 | 2.717 | 1.00 | 30.28 | O |
| ATOM | 2760 | O | HOH | W4041 | 34.765 | −18.840 | 6.665 | 1.00 | 52.31 | O |
| ATOM | 2761 | O | HOH | W4042 | 19.832 | 10.430 | 22.362 | 1.00 | 30.27 | O |
| ATOM | 2762 | O | HOH | W4043 | 25.556 | −0.749 | 36.545 | 1.00 | 47.38 | O |
| ATOM | 2763 | O | HOH | W4044 | 1.280 | −1.711 | 37.119 | 1.00 | 35.25 | O |
| ATOM | 2764 | O | HOH | W4045 | 23.055 | −3.951 | 37.102 | 1.00 | 29.32 | O |
| ATOM | 2765 | O | HOH | W4046 | 10.346 | 9.220 | 18.446 | 1.00 | 31.53 | O |
| ATOM | 2766 | O | HOH | W4047 | −3.287 | 0.403 | 31.271 | 1.00 | 53.94 | O |
| ATOM | 2767 | O | HOH | W4048 | 4.921 | −2.235 | 2.298 | 1.00 | 35.71 | O |
| ATOM | 2768 | O | HOH | W4050 | 4.512 | −5.663 | 22.584 | 1.00 | 34.27 | O |
| ATOM | 2769 | O | HOH | W4051 | 32.165 | −16.448 | 3.541 | 1.00 | 29.46 | O |
| ATOM | 2770 | O | HOH | W4052 | 17.356 | −10.554 | 17.298 | 1.00 | 20.37 | O |
| ATOM | 2771 | O | HOH | W4053 | 23.079 | 8.307 | 36.567 | 1.00 | 27.23 | O |
| ATOM | 2772 | O | HOH | W4054 | 22.333 | 9.961 | 24.703 | 1.00 | 41.93 | O |
| ATOM | 2773 | O | HOH | W4055 | 19.848 | 3.106 | 3.638 | 1.00 | 23.68 | O |
| ATOM | 2774 | O | HOH | W4056 | 14.228 | 9.547 | 2.164 | 1.00 | 13.78 | O |
| ATOM | 2775 | O | HOH | W4057 | −5.992 | 9.409 | 37.088 | 1.00 | 40.47 | O |
| ATOM | 2776 | O | HOH | W4058 | 15.141 | 2.885 | 1.432 | 1.00 | 32.54 | O |
| ATOM | 2777 | O | HOH | W4059 | 27.207 | 3.715 | −0.205 | 1.00 | 38.02 | O |
| ATOM | 2778 | O | HOH | W4060 | 4.520 | 8.617 | 13.683 | 1.00 | 29.66 | O |
| ATOM | 2779 | O | HOH | W4061 | −0.534 | 22.844 | 0.843 | 1.00 | 43.95 | O |
| ATOM | 2780 | O | HOH | W4062 | 33.271 | 0.399 | 4.561 | 1.00 | 41.51 | O |
| ATOM | 2781 | O | HOH | W4063 | 10.750 | −9.990 | 18.523 | 1.00 | 37.79 | O |
| ATOM | 2782 | O | HOH | W4065 | 35.003 | 2.841 | 10.072 | 1.00 | 34.95 | O |
| ATOM | 2783 | O | HOH | W4066 | 15.619 | 13.987 | 20.059 | 1.00 | 51.87 | O |
| ATOM | 2784 | O | HOH | W4067 | 30.322 | −16.861 | 5.985 | 1.00 | 25.67 | O |
| ATOM | 2785 | O | HOH | W4068 | −3.928 | 16.855 | 6.776 | 1.00 | 37.28 | O |
| ATOM | 2786 | O | HOH | W4069 | 20.489 | 9.067 | 39.451 | 1.00 | 48.83 | O |
| ATOM | 2787 | O | HOH | W4070 | 38.865 | −2.991 | 24.079 | 1.00 | 52.94 | O |
| ATOM | 2788 | O | HOH | W4071 | 18.289 | 12.534 | 22.275 | 1.00 | 32.83 | O |
| ATOM | 2789 | O | HOH | W4072 | 16.738 | 0.024 | 14.519 | 1.00 | 34.01 | O |
| ATOM | 2790 | O | HOH | W4073 | 1.959 | −19.500 | 14.371 | 1.00 | 40.61 | O |
| ATOM | 2791 | O | HOH | W4074 | 1.805 | −19.989 | 7.096 | 1.00 | 40.73 | O |
| ATOM | 2792 | O | HOH | W4075 | 15.612 | 10.434 | 34.742 | 1.00 | 32.10 | O |
| ATOM | 2793 | O | HOH | W4076 | 13.088 | 0.232 | 13.391 | 1.00 | 37.71 | O |
| ATOM | 2794 | O | HOH | W4077 | −3.125 | 13.706 | 3.280 | 1.00 | 34.62 | O |
| ATOM | 2795 | O | HOH | W4078 | 2.257 | 8.002 | 15.256 | 1.00 | 30.49 | O |
| ATOM | 2796 | O | HOH | W4079 | 1.805 | 3.820 | 14.707 | 1.00 | 34.86 | O |
| ATOM | 2797 | O | HOH | W4080 | 5.391 | −8.908 | −2.363 | 1.00 | 35.80 | O |
| ATOM | 2798 | O | HOH | W4081 | 13.173 | 18.130 | 19.444 | 1.00 | 42.58 | O |
| ATOM | 2799 | O | HOH | W4083 | 3.428 | 25.760 | 7.166 | 1.00 | 47.46 | O |
| ATOM | 2800 | O | HOH | W4085 | 19.600 | 0.144 | 14.509 | 1.00 | 27.27 | O |
| ATOM | 2801 | O | HOH | W4086 | 17.905 | −21.379 | 25.352 | 1.00 | 38.73 | O |
| ATOM | 2802 | O | HOH | W4087 | 33.360 | 5.719 | 22.662 | 1.00 | 36.03 | O |
| ATOM | 2803 | O | HOH | W4089 | 9.617 | −0.859 | 41.147 | 1.00 | 32.45 | O |
| ATOM | 2804 | O | HOH | W4090 | 28.109 | −16.115 | 0.982 | 1.00 | 29.92 | O |
| ATOM | 2805 | O | HOH | W4091 | 38.387 | −10.896 | 9.764 | 1.00 | 47.94 | O |
| ATOM | 2806 | O | HOH | W4092 | 5.988 | 24.794 | 8.424 | 1.00 | 43.14 | O |
| ATOM | 2807 | O | HOH | W4093 | −3.277 | 6.265 | 6.376 | 1.00 | 35.40 | O |
| ATOM | 2808 | O | HOH | W4094 | 30.455 | 10.041 | 6.941 | 1.00 | 32.99 | O |
| ATOM | 2809 | O | HOH | W4095 | 26.815 | 0.664 | 4.954 | 1.00 | 27.85 | O |
| ATOM | 2810 | O | HOH | W4096 | 15.273 | 23.735 | −4.277 | 1.00 | 47.73 | O |
| ATOM | 2811 | O | HOH | W4099 | 26.449 | 1.509 | 2.656 | 1.00 | 65.15 | O |
| ATOM | 2812 | O | HOH | W4100 | 0.838 | 7.963 | 3.230 | 1.00 | 37.27 | O |
| ATOM | 2813 | O | HOH | W4101 | 29.381 | 8.723 | 0.899 | 1.00 | 43.02 | O |
| ATOM | 2814 | O | HOH | W4102 | 40.660 | −5.785 | 23.570 | 1.00 | 45.63 | O |
| ATOM | 2815 | O | HOH | W4103 | 26.328 | 6.312 | 5.240 | 1.00 | 53.46 | O |
| ATOM | 2816 | O | HOH | W4104 | 34.720 | 1.902 | 16.155 | 1.00 | 43.82 | O |
| ATOM | 2817 | O | HOH | W4105 | −7.319 | −8.388 | 40.539 | 1.00 | 39.00 | O |
| ATOM | 2818 | O | HOH | W4106 | 18.869 | 25.116 | 1.680 | 1.00 | 46.38 | O |
| ATOM | 2819 | O | HOH | W4107 | 16.340 | 14.706 | 24.191 | 1.00 | 38.04 | O |
| ATOM | 2820 | O | HOH | W4108 | 6.923 | 8.908 | 27.885 | 1.00 | 36.28 | O |
| ATOM | 2821 | O | HOH | W4109 | 12.525 | −11.624 | 17.355 | 1.00 | 34.51 | O |
| ATOM | 2822 | O | HOH | W4110 | 34.314 | −7.049 | 18.369 | 1.00 | 34.61 | O |
| ATOM | 2823 | O | HOH | W4113 | 38.750 | −2.512 | 27.167 | 1.00 | 57.88 | O |
| ATOM | 2824 | O | HOH | W4114 | 18.115 | 3.750 | 1.319 | 1.00 | 31.45 | O |
| ATOM | 2825 | O | HOH | W4115 | 27.875 | 1.248 | 31.553 | 1.00 | 55.12 | O |
| ATOM | 2826 | O | HOH | W4116 | 7.683 | −21.130 | −8.639 | 1.00 | 46.38 | O |
| ATOM | 2827 | O | HOH | W4117 | −0.188 | 6.861 | 39.329 | 1.00 | 45.23 | O |
| ATOM | 2828 | O | HOH | W4118 | 15.597 | 2.256 | 13.150 | 1.00 | 38.63 | O |
| ATOM | 2829 | O | HOH | W4120 | 29.648 | 18.094 | 28.167 | 1.00 | 35.93 | O |
| ATOM | 2830 | O | HOH | W4121 | 16.930 | −20.733 | 11.766 | 1.00 | 37.44 | O |
| ATOM | 2831 | O | HOH | W4122 | 7.167 | −12.636 | −7.617 | 1.00 | 48.07 | O |
| ATOM | 2832 | O | HOH | W4123 | 37.628 | 10.036 | 12.640 | 1.00 | 43.48 | O |
| ATOM | 2833 | O | HOH | W4124 | 13.082 | −13.269 | 27.306 | 1.00 | 52.38 | O |
| ATOM | 2834 | O | HOH | W4125 | 4.976 | −26.696 | 6.493 | 1.00 | 47.37 | O |
| ATOM | 2835 | O | HOH | W4126 | 2.241 | −22.225 | 15.163 | 1.00 | 51.68 | O |
| ATOM | 2836 | O | HOH | W4127 | 8.988 | −10.057 | 20.455 | 1.00 | 50.47 | O |
| ATOM | 2837 | O | HOH | W4128 | 18.719 | −13.639 | −6.616 | 1.00 | 49.20 | O |
| ATOM | 2838 | O | HOH | W4129 | 21.651 | 22.882 | 0.638 | 1.00 | 46.89 | O |
| ATOM | 2839 | O | HOH | W4130 | 27.239 | 3.345 | 29.234 | 1.00 | 25.74 | O |
| ATOM | 2840 | O | HOH | W4131 | 9.016 | −8.748 | −1.209 | 1.00 | 45.62 | O |
| ATOM | 2841 | O | HOH | W4132 | 13.082 | −9.632 | 37.155 | 1.00 | 43.55 | O |
| ATOM | 2842 | O | HOH | W4133 | 29.378 | 15.689 | 6.467 | 1.00 | 49.72 | O |
| ATOM | 2843 | O | HOH | W4134 | −7.908 | −3.988 | 42.818 | 1.00 | 30.81 | O |
| ATOM | 2844 | O | HOH | W4135 | 3.562 | −8.297 | 27.665 | 1.00 | 45.56 | O |
| ATOM | 2845 | O | HOH | W4136 | −7.199 | −0.161 | 28.514 | 1.00 | 24.59 | O |
| ATOM | 2846 | O | HOH | W4137 | 31.194 | 6.207 | 6.288 | 1.00 | 42.31 | O |
| ATOM | 2847 | O | HOH | W4138 | 19.383 | 8.002 | 23.933 | 1.00 | 35.57 | O |
| ATOM | 2848 | O | HOH | W4139 | −6.335 | −8.439 | 24.417 | 1.00 | 47.50 | O |
| ATOM | 2849 | O | HOH | W4140 | 37.800 | −1.901 | 17.636 | 1.00 | 38.42 | O |
| ATOM | 2850 | O | HOH | W4141 | 36.896 | 4.279 | 13.213 | 1.00 | 44.41 | O |
| ATOM | 2851 | O | HOH | W4142 | 18.980 | 16.923 | −5.091 | 1.00 | 32.40 | O |
| ATOM | 2852 | O | HOH | W4143 | 6.338 | 17.886 | −7.468 | 1.00 | 44.66 | O |
| ATOM | 2853 | O | HOH | W4144 | 8.099 | 9.789 | −8.452 | 1.00 | 34.29 | O |
| ATOM | 2854 | O | HOH | W4145 | 22.400 | 22.185 | 4.929 | 1.00 | 58.51 | O |
| ATOM | 2855 | O | HOH | W4146 | 23.417 | 1.724 | −2.662 | 1.00 | 49.61 | O |
| ATOM | 2856 | O | HOH | W4147 | 28.353 | 11.186 | 5.638 | 1.00 | 36.80 | O |
| ATOM | 2857 | O | HOH | W4148 | 0.018 | 1.428 | 9.817 | 1.00 | 46.52 | O |
| ATOM | 2858 | O | HOH | W4149 | 38.727 | −0.038 | 26.083 | 1.00 | 50.08 | O |
| ATOM | 2859 | O | HOH | W4150 | 24.171 | −8.880 | 20.179 | 1.00 | 43.36 | O |
| ATOM | 2860 | O | HOH | W4151 | −0.768 | −7.980 | 41.105 | 1.00 | 48.48 | O |
| ATOM | 2861 | O | HOH | W4152 | 26.257 | 5.046 | 35.426 | 1.00 | 48.00 | O |
| ATOM | 2862 | O | HOH | W4153 | 35.432 | 0.075 | 28.104 | 1.00 | 43.89 | O |
| ATOM | 2863 | O | HOH | W4154 | 35.301 | 2.870 | 27.050 | 1.00 | 52.24 | O |
| ATOM | 2864 | O | HOH | W4155 | −4.097 | 3.796 | 17.913 | 1.00 | 44.40 | O |
| ATOM | 2865 | O | HOH | W4156 | 29.195 | 0.822 | 3.786 | 1.00 | 40.55 | O |
| ATOM | 2866 | O | HOH | W4157 | 15.774 | −2.195 | 11.976 | 1.00 | 39.79 | O |
| ATOM | 2867 | O | HOH | W4158 | 22.736 | 4.680 | 3.551 | 1.00 | 24.63 | O |
| ATOM | 2868 | O | HOH | W4159 | 10.086 | −24.038 | 17.929 | 1.00 | 54.44 | O |
| ATOM | 2869 | O | HOH | W4161 | 37.774 | −8.164 | 11.579 | 1.00 | 59.16 | O |
| ATOM | 2870 | O | HOH | W4162 | 25.882 | 18.452 | 33.331 | 1.00 | 33.59 | O |
| ATOM | 2871 | O | HOH | W4163 | −0.430 | 0.356 | 12.041 | 1.00 | 45.32 | O |
| ATOM | 2872 | O | HOH | W4164 | −3.073 | −7.128 | 24.073 | 1.00 | 45.12 | O |
| ATOM | 2873 | O | HOH | W4165 | 31.655 | 13.815 | 8.848 | 1.00 | 29.05 | O |
| ATOM | 2874 | O | HOH | W4166 | 17.133 | 8.014 | −0.782 | 1.00 | 24.81 | O |
| ATOM | 2875 | O | HOH | W4167 | 11.582 | −12.517 | 13.880 | 1.00 | 37.19 | O |
| ATOM | 2876 | O | HOH | W4168 | 15.057 | −15.827 | 14.902 | 1.00 | 25.73 | O |
| ATOM | 2877 | O | HOH | W4169 | 12.130 | −17.876 | 15.236 | 1.00 | 35.36 | O |
| ATOM | 2878 | O | HOH | W4170 | 7.663 | 8.075 | 46.234 | 1.00 | 31.45 | O |
| ATOM | 2879 | O | HOH | W4171 | 8.858 | 17.412 | 12.301 | 1.00 | 37.47 | O |
| TER | 2880 | | HOH | W4171 | | | | | | |
| END | | | | | | | | | | |

TABLE 5
REMARK coordinates from restrained individual B-factor refinement
REMARK refinement resolution: 500.0 − 1.90 A
REMARK starting r = 0.2427 free_r = 0.2720
REMARK final r = 0.2425 free_r = 0.2731
REMARK B rmsd for bonded mainchain atoms = 0.960 target = 1.5
REMARK B rmsd for bonded sidechain atoms = 1.085 target = 2.0
REMARK B rmsd for angle mainchain atoms = 1.688 target = 2.0
REMARK B rmsd for angle sidechain atoms = 1.768 target = 2.5
REMARK wa = 1.18715
REMARK rweight = 0.298329
REMARK target = mlf steps = 30
REMARK sg = C2 a = 120.188 b = 62.273 c = 51.673 alpha = 90
beta = 98.745 gamma = 90
REMARK parameter file 1 CNX_TOPPAR:protein_rep.param
REMARK parameter file 2 CNX_TOPPAR:water_rep.param
REMARK parameter file 3 CNX_TOPPAR:ion.param
REMARK parameter file 4 35-ADP/35-ADP.param
REMARK molecular structure file: generate.psf
REMARK input coordinates: minimize.pdb
REMARK reflection file = ../data/x-ray_data.cv
REMARK ncs = none
REMARK B-correction resolution: 6.0 − 1.90
REMARK initial B-factor correction applied to fobs:
REMARK B11 = 0.036 B22 = −2.635 B33 = 2.599
REMARK B12 = 0.000 B13 = 0.515 B23 = 0.000
REMARK B-factor correction applied to coordinate array B: 0.030
REMARK bulk solvent: (Mask) density level = 0.369861 e/A\hat{ }3, B-factor = 96.7512 A\hat{ }2
REMARK reflections with |Fobs|/sigma_F < 0.0 rejected
REMARK reflections with |Fobs| > 10000 * rms (Fobs) rejected

| | | |
|---|---|---|
| REMARK theoretical total number of refl. in resol. range: | 29870 | (100.0%) |
| REMARK number of unobserved reflections (no entry or \|F\| = 0): | 330 | (1.1%) |
| REMARK number of reflections rejected: | 0 | (0.0%) |
| REMARK total number of reflections used: | 29540 | (98.9%) |
| REMARK number of reflections in working set: | 28083 | (94.0%) |
| REMARK number of reflections in test set: | 1457 | (4.9%) |

REMARK FILENAME = “acps 35-ADP.pdb”
REMARK Written by CNX VERSION: 2000

| | | | | | | | | | | |
|---|---|---|---|---|---|---|---|---|---|---|
| ATOM | 1 | CB | MET | A1003 | 37.264 | 5.634 | 20.230 | 1.00 | 26.46 | C |
| ATOM | 2 | CG | MET | A1003 | 36.648 | 6.209 | 21.498 | 1.00 | 29.51 | C |
| ATOM | 3 | SD | MET | A1003 | 37.784 | 7.312 | 22.398 | 1.00 | 33.70 | S |
| ATOM | 4 | CE | MET | A1003 | 37.843 | 8.718 | 21.291 | 1.00 | 32.08 | C |
| ATOM | 5 | C | MET | A1003 | 35.048 | 5.262 | 19.102 | 1.00 | 23.04 | C |
| ATOM | 6 | O | MET | A1003 | 35.009 | 6.245 | 18.358 | 1.00 | 22.46 | O |
| ATOM | 7 | N | MET | A1003 | 37.108 | 4.155 | 18.256 | 1.00 | 24.80 | N |
| ATOM | 8 | CA | MET | A1003 | 36.389 | 4.622 | 19.479 | 1.00 | 24.32 | C |
| ATOM | 9 | N | ILE | A1004 | 33.957 | 4.690 | 19.606 | 1.00 | 22.15 | N |
| ATOM | 10 | CA | ILE | A1004 | 32.606 | 5.202 | 19.348 | 1.00 | 21.16 | C |
| ATOM | 11 | CB | ILE | A1004 | 31.535 | 4.120 | 19.670 | 1.00 | 21.51 | C |
| ATOM | 12 | CG2 | ILE | A1004 | 30.122 | 4.710 | 19.558 | 1.00 | 21.66 | C |
| ATOM | 13 | CG1 | ILE | A1004 | 31.696 | 2.927 | 18.722 | 1.00 | 21.75 | C |
| ATOM | 14 | CD1 | ILE | A1004 | 30.823 | 1.734 | 19.074 | 1.00 | 22.20 | C |
| ATOM | 15 | C | ILE | A1004 | 32.361 | 6.417 | 20.245 | 1.00 | 20.57 | C |
| ATOM | 16 | O | ILE | A1004 | 32.773 | 6.416 | 21.407 | 1.00 | 21.36 | O |
| ATOM | 17 | N | VAL | A1005 | 31.705 | 7.448 | 19.713 | 1.00 | 19.47 | N |
| ATOM | 18 | CA | VAL | A1005 | 31.409 | 8.652 | 20.492 | 1.00 | 18.73 | C |
| ATOM | 19 | CB | VAL | A1005 | 32.308 | 9.852 | 20.042 | 1.00 | 19.66 | C |
| ATOM | 20 | CG1 | VAL | A1005 | 33.779 | 9.530 | 20.319 | 1.00 | 20.39 | C |
| ATOM | 21 | CG2 | VAL | A1005 | 32.119 | 10.142 | 18.562 | 1.00 | 20.39 | C |
| ATOM | 22 | C | VAL | A1005 | 29.924 | 9.085 | 20.502 | 1.00 | 17.55 | C |
| ATOM | 23 | O | VAL | A1005 | 29.601 | 10.193 | 20.940 | 1.00 | 16.98 | O |
| ATOM | 24 | N | GLY | A1006 | 29.026 | 8.207 | 20.041 | 1.00 | 16.14 | N |
| ATOM | 25 | CA | GLY | A1006 | 27.593 | 8.513 | 20.046 | 1.00 | 14.83 | C |
| ATOM | 26 | C | GLY | A1006 | 26.725 | 7.566 | 19.216 | 1.00 | 13.78 | C |
| ATOM | 27 | O | GLY | A1006 | 27.223 | 6.982 | 18.257 | 1.00 | 12.47 | O |
| ATOM | 28 | N | HIS | A1007 | 25.442 | 7.409 | 19.581 | 1.00 | 13.88 | N |
| ATOM | 29 | CA | HIS | A1007 | 24.495 | 6.535 | 18.844 | 1.00 | 14.19 | C |
| ATOM | 30 | CB | HIS | A1007 | 24.638 | 5.080 | 19.332 | 1.00 | 14.36 | C |
| ATOM | 31 | CG | HIS | A1007 | 23.663 | 4.115 | 18.711 | 1.00 | 14.94 | C |
| ATOM | 32 | CD2 | HIS | A1007 | 22.904 | 3.137 | 19.266 | 1.00 | 15.52 | C |
| ATOM | 33 | ND1 | HIS | A1007 | 23.410 | 4.070 | 17.355 | 1.00 | 14.28 | N |
| ATOM | 34 | CE1 | HIS | A1007 | 22.537 | 3.108 | 17.102 | 1.00 | 15.47 | C |
| ATOM | 35 | NE2 | HIS | A1007 | 22.214 | 2.526 | 18.245 | 1.00 | 15.49 | N |
| ATOM | 36 | C | HIS | A1007 | 23.025 | 7.000 | 18.999 | 1.00 | 13.90 | C |
| ATOM | 37 | O | HIS | A1007 | 22.588 | 7.310 | 20.115 | 1.00 | 13.25 | O |
| ATOM | 38 | N | GLY | A1008 | 22.276 | 7.053 | 17.888 | 1.00 | 13.72 | N |
| ATOM | 39 | CA | GLY | A1008 | 20.870 | 7.476 | 17.931 | 1.00 | 13.61 | C |
| ATOM | 40 | C | GLY | A1008 | 19.944 | 6.833 | 16.890 | 1.00 | 13.86 | C |
| ATOM | 41 | O | GLY | A1008 | 20.402 | 6.498 | 15.795 | 1.00 | 13.86 | O |
| ATOM | 42 | N | ILE | A1009 | 18.653 | 6.649 | 17.218 | 1.00 | 13.29 | N |
| ATOM | 43 | CA | ILE | A1009 | 17.672 | 6.046 | 16.281 | 1.00 | 13.44 | C |
| ATOM | 44 | CB | ILE | A1009 | 17.342 | 4.554 | 16.639 | 1.00 | 14.13 | C |
| ATOM | 45 | CG2 | ILE | A1009 | 18.619 | 3.696 | 16.642 | 1.00 | 13.71 | C |
| ATOM | 46 | CG1 | ILE | A1009 | 16.638 | 4.503 | 18.003 | 1.00 | 13.45 | C |
| ATOM | 47 | CD1 | ILE | A1009 | 16.175 | 3.134 | 18.434 | 1.00 | 15.16 | C |
| ATOM | 48 | C | ILE | A1009 | 16.318 | 6.799 | 16.257 | 1.00 | 14.09 | C |
| ATOM | 49 | O | ILE | A1009 | 16.033 | 7.589 | 17.165 | 1.00 | 12.83 | O |
| ATOM | 50 | N | ASP | A1010 | 15.491 | 6.537 | 15.232 | 1.00 | 14.68 | N |
| ATOM | 51 | CA | ASP | A1010 | 14.161 | 7.167 | 15.101 | 1.00 | 16.24 | C |
| ATOM | 52 | CB | ASP | A1010 | 14.287 | 8.580 | 14.496 | 1.00 | 17.11 | C |
| ATOM | 53 | CG | ASP | A1010 | 12.944 | 9.328 | 14.428 | 1.00 | 19.40 | C |
| ATOM | 54 | OD1 | ASP | A1010 | 12.265 | 9.295 | 13.369 | 1.00 | 19.82 | O |
| ATOM | 55 | OD2 | ASP | A1010 | 12.563 | 9.948 | 15.446 | 1.00 | 19.26 | O |
| ATOM | 56 | C | ASP | A1010 | 13.193 | 6.348 | 14.230 | 1.00 | 16.72 | C |
| ATOM | 57 | O | ASP | A1010 | 13.618 | 5.693 | 13.276 | 1.00 | 16.89 | O |
| ATOM | 58 | N | ILE | A1011 | 11.900 | 6.387 | 14.570 | 1.00 | 17.01 | N |
| ATOM | 59 | CA | ILE | A1011 | 10.850 | 5.706 | 13.796 | 1.00 | 17.33 | C |
| ATOM | 60 | CB | ILE | A1011 | 10.410 | 4.353 | 14.447 | 1.00 | 18.10 | C |
| ATOM | 61 | CG2 | ILE | A1011 | 9.963 | 4.570 | 15.898 | 1.00 | 17.28 | C |
| ATOM | 62 | CG1 | ILE | A1011 | 9.297 | 3.717 | 13.601 | 1.00 | 18.21 | C |
| ATOM | 63 | CD1 | ILE | A1011 | 8.852 | 2.338 | 14.083 | 1.00 | 20.73 | C |
| ATOM | 64 | C | ILE | A1011 | 9.648 | 6.664 | 13.679 | 1.00 | 18.00 | C |
| ATOM | 65 | O | ILE | A1011 | 9.262 | 7.298 | 14.663 | 1.00 | 16.80 | O |
| ATOM | 66 | N | GLU | A1012 | 9.060 | 6.760 | 12.481 | 1.00 | 18.22 | N |
| ATOM | 67 | CA | GLU | A1012 | 7.951 | 7.690 | 12.220 | 1.00 | 19.38 | C |
| ATOM | 68 | CB | GLU | A1012 | 8.537 | 8.972 | 11.609 | 1.00 | 20.91 | C |
| ATOM | 69 | CG | GLU | A1012 | 7.537 | 10.036 | 11.186 | 1.00 | 23.69 | C |
| ATOM | 70 | CD | GLU | A1012 | 7.065 | 10.909 | 12.336 | 1.00 | 24.81 | C |
| ATOM | 71 | OE1 | GLU | A1012 | 7.432 | 10.632 | 13.499 | 1.00 | 24.64 | O |
| ATOM | 72 | OE2 | GLU | A1012 | 6.318 | 11.878 | 12.070 | 1.00 | 27.04 | O |
| ATOM | 73 | C | GLU | A1012 | 6.845 | 7.146 | 11.293 | 1.00 | 19.45 | C |
| ATOM | 74 | O | GLU | A1012 | 7.119 | 6.397 | 10.358 | 1.00 | 17.84 | O |
| ATOM | 75 | N | GLU | A1013 | 5.599 | 7.538 | 11.557 | 1.00 | 20.13 | N |
| ATOM | 76 | CA | GLU | A1013 | 4.456 | 7.119 | 10.735 | 1.00 | 21.62 | C |
| ATOM | 77 | CB | GLU | A1013 | 3.178 | 7.146 | 11.584 | 1.00 | 24.29 | C |
| ATOM | 78 | CG | GLU | A1013 | 1.916 | 6.753 | 10.845 | 1.00 | 28.34 | C |
| ATOM | 79 | CD | GLU | A1013 | 0.710 | 6.656 | 11.766 | 1.00 | 30.69 | C |
| ATOM | 80 | OE1 | GLU | A1013 | −0.400 | 6.361 | 11.267 | 1.00 | 33.13 | O |
| ATOM | 81 | OE2 | GLU | A1013 | 0.871 | 6.872 | 12.990 | 1.00 | 33.49 | O |
| ATOM | 82 | C | GLU | A1013 | 4.302 | 8.061 | 9.521 | 1.00 | 21.59 | C |
| ATOM | 83 | O | GLU | A1013 | 4.247 | 9.280 | 9.682 | 1.00 | 20.05 | O |
| ATOM | 84 | N | LEU | A1014 | 4.223 | 7.493 | 8.316 | 1.00 | 21.61 | N |
| ATOM | 85 | CA | LEU | A1014 | 4.104 | 8.288 | 7.087 | 1.00 | 22.02 | C |
| ATOM | 86 | CB | LEU | A1014 | 4.191 | 7.365 | 5.857 | 1.00 | 23.16 | C |
| ATOM | 87 | CG | LEU | A1014 | 5.502 | 6.571 | 5.745 | 1.00 | 24.38 | C |
| ATOM | 88 | CD1 | LEU | A1014 | 5.454 | 5.622 | 4.551 | 1.00 | 25.08 | C |
| ATOM | 89 | CD2 | LEU | A1014 | 6.667 | 7.538 | 5.604 | 1.00 | 25.38 | C |
| ATOM | 90 | C | LEU | A1014 | 2.841 | 9.158 | 7.006 | 1.00 | 22.02 | C |
| ATOM | 91 | O | LEU | A1014 | 2.867 | 10.240 | 6.419 | 1.00 | 21.55 | O |
| ATOM | 92 | N | ALA | A1015 | 1.742 | 8.691 | 7.593 | 1.00 | 22.06 | N |
| ATOM | 93 | CA | ALA | A1015 | 0.488 | 9.452 | 7.595 | 1.00 | 22.62 | C |
| ATOM | 94 | CB | ALA | A1015 | −0.605 | 8.656 | 8.299 | 1.00 | 22.21 | C |
| ATOM | 95 | C | ALA | A1015 | 0.644 | 10.817 | 8.274 | 1.00 | 22.87 | C |
| ATOM | 96 | O | ALA | A1015 | 0.005 | 11.796 | 7.875 | 1.00 | 23.00 | O |
| ATOM | 97 | N | SER | A1016 | 1.482 | 10.876 | 9.307 | 1.00 | 23.27 | N |
| ATOM | 98 | CA | SER | A1016 | 1.716 | 12.123 | 10.034 | 1.00 | 23.77 | C |
| ATOM | 99 | CB | SER | A1016 | 2.535 | 11.860 | 11.305 | 1.00 | 24.30 | C |
| ATOM | 100 | OG | SER | A1016 | 1.890 | 10.919 | 12.145 | 1.00 | 26.09 | O |
| ATOM | 101 | C | SER | A1016 | 2.443 | 13.146 | 9.162 | 1.00 | 23.41 | C |
| ATOM | 102 | O | SER | A1016 | 2.173 | 14.348 | 9.249 | 1.00 | 23.19 | O |
| ATOM | 103 | N | ILE | A1017 | 3.378 | 12.673 | 8.340 | 1.00 | 23.05 | N |
| ATOM | 104 | CA | ILE | A1017 | 4.118 | 13.566 | 7.450 | 1.00 | 22.92 | C |
| ATOM | 105 | CB | ILE | A1017 | 5.355 | 12.860 | 6.838 | 1.00 | 23.00 | C |
| ATOM | 106 | CG2 | ILE | A1017 | 6.107 | 13.823 | 5.918 | 1.00 | 23.26 | C |
| ATOM | 107 | CG1 | ILE | A1017 | 6.289 | 12.365 | 7.951 | 1.00 | 23.63 | C |
| ATOM | 108 | CD1 | ILE | A1017 | 6.896 | 13.467 | 8.810 | 1.00 | 22.84 | C |
| ATOM | 109 | C | ILE | A1017 | 3.204 | 14.053 | 6.308 | 1.00 | 23.44 | C |
| ATOM | 110 | O | ILE | A1017 | 3.276 | 15.218 | 5.896 | 1.00 | 23.11 | O |
| ATOM | 111 | N | GLU | A1018 | 2.341 | 13.170 | 5.805 | 1.00 | 22.98 | N |
| ATOM | 112 | CA | GLU | A1018 | 1.443 | 13.549 | 4.714 | 1.00 | 24.09 | C |
| ATOM | 113 | CB | GLU | A1018 | 0.729 | 12.306 | 4.147 | 1.00 | 25.23 | C |
| ATOM | 114 | CG | GLU | A1018 | −0.252 | 12.592 | 2.997 | 1.00 | 27.75 | C |
| ATOM | 115 | CD | GLU | A1018 | −0.632 | 11.345 | 2.190 | 1.00 | 29.64 | C |
| ATOM | 116 | OE1 | GLU | A1018 | −0.807 | 10.255 | 2.783 | 1.00 | 30.50 | O |
| ATOM | 117 | OE2 | GLU | A1018 | −0.771 | 11.459 | 0.952 | 1.00 | 30.61 | O |
| ATOM | 118 | C | GLU | A1018 | 0.427 | 14.612 | 5.150 | 1.00 | 24.15 | C |
| ATOM | 119 | O | GLU | A1018 | 0.142 | 15.550 | 4.395 | 1.00 | 23.68 | O |
| ATOM | 120 | N | SER | A1019 | −0.103 | 14.481 | 6.367 | 1.00 | 23.86 | N |
| ATOM | 121 | CA | SER | A1019 | −1.078 | 15.445 | 6.879 | 1.00 | 24.87 | C |
| ATOM | 122 | CB | SER | A1019 | −1.641 | 14.993 | 8.234 | 1.00 | 24.60 | C |
| ATOM | 123 | OG | SER | A1019 | −2.387 | 13.794 | 8.120 | 1.00 | 26.37 | O |
| ATOM | 124 | C | SER | A1019 | −0.450 | 16.824 | 7.045 | 1.00 | 25.06 | C |
| ATOM | 125 | O | SER | A1019 | −1.077 | 17.836 | 6.741 | 1.00 | 25.39 | O |
| ATOM | 126 | N | ALA | A1020 | 0.790 | 16.856 | 7.525 | 1.00 | 25.98 | N |
| ATOM | 127 | CA | ALA | A1020 | 1.508 | 18.112 | 7.748 | 1.00 | 26.26 | C |
| ATOM | 128 | CB | ALA | A1020 | 2.869 | 17.829 | 8.399 | 1.00 | 26.48 | C |
| ATOM | 129 | C | ALA | A1020 | 1.695 | 18.909 | 6.460 | 1.00 | 26.83 | C |
| ATOM | 130 | O | ALA | A1020 | 1.589 | 20.138 | 6.463 | 1.00 | 26.25 | O |
| ATOM | 131 | N | VAL | A1021 | 1.974 | 18.210 | 5.364 | 1.00 | 27.31 | N |
| ATOM | 132 | CA | VAL | A1021 | 2.166 | 18.855 | 4.064 | 1.00 | 28.77 | C |
| ATOM | 133 | CB | VAL | A1021 | 2.767 | 17.868 | 3.029 | 1.00 | 28.35 | C |
| ATOM | 134 | CG1 | VAL | A1021 | 2.801 | 18.510 | 1.639 | 1.00 | 28.63 | C |
| ATOM | 135 | CG2 | VAL | A1021 | 4.176 | 17.461 | 3.457 | 1.00 | 28.09 | C |
| ATOM | 136 | C | VAL | A1021 | 0.845 | 19.396 | 3.512 | 1.00 | 30.16 | C |
| ATOM | 137 | O | VAL | A1021 | 0.775 | 20.541 | 3.054 | 1.00 | 29.31 | O |
| ATOM | 138 | N | THR | A1022 | −0.195 | 18.565 | 3.557 | 1.00 | 31.76 | N |
| ATOM | 139 | CA | THR | A1022 | −1.515 | 18.951 | 3.057 | 1.00 | 33.81 | C |
| ATOM | 140 | CB | THR | A1022 | −2.541 | 17.798 | 3.230 | 1.00 | 33.53 | C |
| ATOM | 141 | OG1 | THR | A1022 | −2.119 | 16.663 | 2.466 | 1.00 | 33.79 | O |
| ATOM | 142 | CG2 | THR | A1022 | −3.926 | 18.224 | 2.749 | 1.00 | 33.72 | C |
| ATOM | 143 | C | THR | A1022 | −2.032 | 20.183 | 3.790 | 1.00 | 34.97 | C |
| ATOM | 144 | O | THR | A1022 | −2.802 | 20.968 | 3.237 | 1.00 | 36.01 | O |
| ATOM | 145 | N | ARG | A1023 | −1.585 | 20.346 | 5.029 | 1.00 | 36.61 | N |
| ATOM | 146 | CA | ARG | A1023 | −1.988 | 21.462 | 5.876 | 1.00 | 38.01 | C |
| ATOM | 147 | CB | ARG | A1023 | −1.371 | 21.297 | 7.267 | 1.00 | 38.96 | C |
| ATOM | 148 | CG | ARG | A1023 | −2.284 | 21.702 | 8.409 | 1.00 | 40.44 | C |
| ATOM | 149 | CD | ARG | A1023 | −2.443 | 20.556 | 9.393 | 1.00 | 41.63 | C |
| ATOM | 150 | NE | ARG | A1023 | −1.184 | 20.219 | 10.053 | 1.00 | 42.83 | N |
| ATOM | 151 | CZ | ARG | A1023 | −1.000 | 19.134 | 10.800 | 1.00 | 43.45 | C |
| ATOM | 152 | NH1 | ARG | A1023 | −1.997 | 18.277 | 10.981 | 1.00 | 43.85 | N |
| ATOM | 153 | NH2 | ARG | A1023 | 0.178 | 18.906 | 11.367 | 1.00 | 43.30 | N |
| ATOM | 154 | C | ARG | A1023 | −1.603 | 22.825 | 5.302 | 1.00 | 38.55 | C |
| ATOM | 155 | O | ARG | A1023 | −2.143 | 23.850 | 5.722 | 1.00 | 38.81 | O |
| ATOM | 156 | N | HIS | A1024 | −0.663 | 22.842 | 4.359 | 1.00 | 38.91 | N |
| ATOM | 157 | CA | HIS | A1024 | −0.244 | 24.096 | 3.733 | 1.00 | 38.99 | C |
| ATOM | 158 | CB | HIS | A1024 | −1.432 | 24.654 | 2.935 | 1.00 | 40.09 | C |
| ATOM | 159 | CG | HIS | A1024 | −1.184 | 25.993 | 2.315 | 1.00 | 41.43 | C |
| ATOM | 160 | CD2 | HIS | A1024 | −1.936 | 27.119 | 2.304 | 1.00 | 41.88 | C |
| ATOM | 161 | ND1 | HIS | A1024 | −0.058 | 26.276 | 1.573 | 1.00 | 41.78 | N |
| ATOM | 162 | CE1 | HIS | A1024 | −0.126 | 27.519 | 1.131 | 1.00 | 42.17 | C |
| ATOM | 163 | NE2 | HIS | A1024 | −1.257 | 28.053 | 1.560 | 1.00 | 42.70 | N |
| ATOM | 164 | C | HIS | A1024 | 0.245 | 25.107 | 4.782 | 1.00 | 38.60 | C |
| ATOM | 165 | O | HIS | A1024 | −0.251 | 26.236 | 4.857 | 1.00 | 38.61 | O |
| ATOM | 166 | N | GLU | A1025 | 1.234 | 24.696 | 5.575 | 1.00 | 37.69 | N |
| ATOM | 167 | CA | GLU | A1025 | 1.784 | 25.533 | 6.643 | 1.00 | 36.89 | C |
| ATOM | 168 | CB | GLU | A1025 | 1.545 | 24.854 | 7.995 | 1.00 | 37.94 | C |
| ATOM | 169 | CG | GLU | A1025 | 2.255 | 23.508 | 8.126 | 1.00 | 39.13 | C |
| ATOM | 170 | CD | GLU | A1025 | 2.003 | 22.828 | 9.459 | 1.00 | 40.28 | C |
| ATOM | 171 | OE1 | GLU | A1025 | 0.853 | 22.405 | 9.709 | 1.00 | 41.24 | O |
| ATOM | 172 | OE2 | GLU | A1025 | 2.959 | 22.716 | 10.258 | 1.00 | 40.84 | O |
| ATOM | 173 | C | GLU | A1025 | 3.280 | 25.856 | 6.518 | 1.00 | 35.45 | C |
| ATOM | 174 | O | GLU | A1025 | 3.857 | 26.466 | 7.424 | 1.00 | 35.71 | O |
| ATOM | 175 | N | GLY | A1026 | 3.906 | 25.441 | 5.420 | 1.00 | 33.22 | N |
| ATOM | 176 | CA | GLY | A1026 | 5.324 | 25.708 | 5.220 | 1.00 | 30.99 | C |
| ATOM | 177 | C | GLY | A1026 | 6.265 | 24.668 | 5.819 | 1.00 | 29.44 | C |
| ATOM | 178 | O | GLY | A1026 | 7.448 | 24.943 | 6.040 | 1.00 | 28.80 | O |
| ATOM | 179 | N | PHE | A1027 | 5.743 | 23.470 | 6.073 | 1.00 | 27.49 | N |
| ATOM | 180 | CA | PHE | A1027 | 6.524 | 22.378 | 6.657 | 1.00 | 26.42 | C |
| ATOM | 181 | CB | PHE | A1027 | 5.618 | 21.149 | 6.830 | 1.00 | 26.50 | C |
| ATOM | 182 | CG | PHE | A1027 | 6.272 | 19.995 | 7.541 | 1.00 | 26.07 | C |
| ATOM | 183 | CD1 | PHE | A1027 | 6.650 | 20.103 | 8.876 | 1.00 | 25.99 | C |
| ATOM | 184 | CD2 | PHE | A1027 | 6.499 | 18.790 | 6.873 | 1.00 | 26.17 | C |
| ATOM | 185 | CE1 | PHE | A1027 | 7.243 | 19.028 | 9.541 | 1.00 | 26.70 | C |
| ATOM | 186 | CE2 | PHE | A1027 | 7.091 | 17.711 | 7.527 | 1.00 | 26.08 | C |
| ATOM | 187 | CZ | PHE | A1027 | 7.465 | 17.828 | 8.863 | 1.00 | 26.23 | C |
| ATOM | 188 | C | PHE | A1027 | 7.776 | 21.998 | 5.852 | 1.00 | 25.70 | C |
| ATOM | 189 | O | PHE | A1027 | 8.892 | 22.017 | 6.377 | 1.00 | 25.54 | O |
| ATOM | 190 | N | ALA | A1028 | 7.598 | 21.656 | 4.579 | 1.00 | 24.86 | N |
| ATOM | 191 | CA | ALA | A1028 | 8.726 | 21.260 | 3.737 | 1.00 | 24.85 | C |
| ATOM | 192 | CB | ALA | A1028 | 8.232 | 20.919 | 2.333 | 1.00 | 24.87 | C |
| ATOM | 193 | C | ALA | A1028 | 9.824 | 22.325 | 3.661 | 1.00 | 25.07 | C |
| ATOM | 194 | O | ALA | A1028 | 11.018 | 22.009 | 3.685 | 1.00 | 24.02 | O |
| ATOM | 195 | N | LYS | A1029 | 9.417 | 23.587 | 3.571 | 1.00 | 25.80 | N |
| ATOM | 196 | CA | LYS | A1029 | 10.364 | 24.695 | 3.488 | 1.00 | 26.80 | C |
| ATOM | 197 | CB | LYS | A1029 | 9.598 | 26.000 | 3.222 | 1.00 | 28.18 | C |
| ATOM | 198 | CG | LYS | A1029 | 10.449 | 27.200 | 2.800 | 1.00 | 30.30 | C |
| ATOM | 199 | CD | LYS | A1029 | 11.251 | 27.769 | 3.958 | 1.00 | 32.28 | C |
| ATOM | 200 | CE | LYS | A1029 | 11.799 | 29.151 | 3.636 | 1.00 | 33.23 | C |
| ATOM | 201 | NZ | LYS | A1029 | 10.702 | 30.153 | 3.506 | 1.00 | 34.55 | N |
| ATOM | 202 | C | LYS | A1029 | 11.197 | 24.806 | 4.771 | 1.00 | 26.54 | C |
| ATOM | 203 | O | LYS | A1029 | 12.344 | 25.244 | 4.738 | 1.00 | 26.39 | O |
| ATOM | 204 | N | ARG | A1030 | 10.624 | 24.394 | 5.897 | 1.00 | 26.43 | N |
| ATOM | 205 | CA | ARG | A1030 | 11.327 | 24.464 | 7.178 | 1.00 | 26.50 | C |
| ATOM | 206 | CB | ARG | A1030 | 10.312 | 24.485 | 8.334 | 1.00 | 27.57 | C |
| ATOM | 207 | CG | ARG | A1030 | 10.945 | 24.487 | 9.727 | 1.00 | 30.51 | C |
| ATOM | 208 | CD | ARG | A1030 | 9.933 | 24.858 | 10.820 | 1.00 | 31.96 | C |
| ATOM | 209 | NE | ARG | A1030 | 8.887 | 23.851 | 11.008 | 1.00 | 32.84 | N |
| ATOM | 210 | CZ | ARG | A1030 | 9.033 | 22.740 | 11.723 | 1.00 | 33.04 | C |
| ATOM | 211 | NH1 | ARG | A1030 | 10.184 | 22.482 | 12.327 | 1.00 | 33.30 | N |
| ATOM | 212 | NH2 | ARG | A1030 | 8.028 | 21.884 | 11.836 | 1.00 | 33.60 | N |
| ATOM | 213 | C | ARG | A1030 | 12.325 | 23.327 | 7.407 | 1.00 | 25.55 | C |
| ATOM | 214 | O | ARG | A1030 | 13.402 | 23.539 | 7.976 | 1.00 | 25.72 | O |
| ATOM | 215 | N | VAL | A1031 | 11.967 | 22.130 | 6.952 | 1.00 | 24.16 | N |
| ATOM | 216 | CA | VAL | A1031 | 12.795 | 20.934 | 7.134 | 1.00 | 23.09 | C |
| ATOM | 217 | CB | VAL | A1031 | 11.903 | 19.655 | 7.150 | 1.00 | 23.53 | C |
| ATOM | 218 | CG1 | VAL | A1031 | 12.765 | 18.398 | 7.339 | 1.00 | 23.93 | C |
| ATOM | 219 | CG2 | VAL | A1031 | 10.874 | 19.754 | 8.258 | 1.00 | 22.96 | C |
| ATOM | 220 | C | VAL | A1031 | 13.932 | 20.684 | 6.134 | 1.00 | 22.57 | C |
| ATOM | 221 | O | VAL | A1031 | 14.952 | 20.094 | 6.504 | 1.00 | 22.31 | O |
| ATOM | 222 | N | LEU | A1032 | 13.765 | 21.127 | 4.887 | 1.00 | 21.47 | N |
| ATOM | 223 | CA | LEU | A1032 | 14.764 | 20.888 | 3.830 | 1.00 | 21.78 | C |
| ATOM | 224 | CB | LEU | A1032 | 14.062 | 20.277 | 2.607 | 1.00 | 21.38 | C |
| ATOM | 225 | CG | LEU | A1032 | 13.126 | 19.073 | 2.773 | 1.00 | 20.75 | C |
| ATOM | 226 | CD1 | LEU | A1032 | 12.446 | 18.779 | 1.428 | 1.00 | 21.22 | C |
| ATOM | 227 | CD2 | LEU | A1032 | 13.912 | 17.851 | 3.249 | 1.00 | 20.06 | C |
| ATOM | 228 | C | LEU | A1032 | 15.565 | 22.107 | 3.355 | 1.00 | 22.39 | C |
| ATOM | 229 | O | LEU | A1032 | 15.061 | 23.233 | 3.375 | 1.00 | 22.40 | O |
| ATOM | 230 | N | THR | A1033 | 16.806 | 21.875 | 2.917 | 1.00 | 23.26 | N |
| ATOM | 231 | CA | THR | A1033 | 17.661 | 22.951 | 2.394 | 1.00 | 24.31 | C |
| ATOM | 232 | CB | THR | A1033 | 19.171 | 22.577 | 2.366 | 1.00 | 24.32 | C |
| ATOM | 233 | OG1 | THR | A1033 | 19.380 | 21.472 | 1.473 | 1.00 | 24.43 | O |
| ATOM | 234 | CG2 | THR | A1033 | 19.671 | 22.217 | 3.756 | 1.00 | 23.93 | C |
| ATOM | 235 | C | THR | A1033 | 17.255 | 23.215 | 0.945 | 1.00 | 25.20 | C |
| ATOM | 236 | O | THR | A1033 | 16.343 | 22.570 | 0.426 | 1.00 | 24.87 | O |
| ATOM | 237 | N | ALA | A1034 | 17.937 | 24.158 | 0.297 | 1.00 | 25.70 | N |
| ATOM | 238 | CA | ALA | A1034 | 17.650 | 24.476 | −1.100 | 1.00 | 26.57 | C |
| ATOM | 239 | CB | ALA | A1034 | 18.485 | 25.676 | −1.548 | 1.00 | 27.15 | C |
| ATOM | 240 | C | ALA | A1034 | 17.943 | 23.270 | −1.997 | 1.00 | 26.78 | C |
| ATOM | 241 | O | ALA | A1034 | 17.115 | 22.896 | −2.829 | 1.00 | 27.20 | O |
| ATOM | 242 | N | LEU | A1035 | 19.122 | 22.672 | −1.831 | 1.00 | 26.52 | N |
| ATOM | 243 | CA | LEU | A1035 | 19.513 | 21.503 | −2.623 | 1.00 | 27.27 | C |
| ATOM | 244 | CB | LEU | A1035 | 20.943 | 21.063 | −2.276 | 1.00 | 27.34 | C |
| ATOM | 245 | CG | LEU | A1035 | 22.137 | 21.950 | −2.658 | 1.00 | 28.71 | C |
| ATOM | 246 | CD1 | LEU | A1035 | 23.388 | 21.443 | −1.953 | 1.00 | 28.34 | C |
| ATOM | 247 | CD2 | LEU | A1035 | 22.348 | 21.949 | −4.171 | 1.00 | 29.34 | C |
| ATOM | 248 | C | LEU | A1035 | 18.549 | 20.327 | −2.408 | 1.00 | 26.73 | C |
| ATOM | 249 | O | LEU | A1035 | 18.124 | 19.679 | −3.369 | 1.00 | 26.11 | O |
| ATOM | 250 | N | GLU | A1036 | 18.209 | 20.048 | −1.151 | 1.00 | 26.36 | N |
| ATOM | 251 | CA | GLU | A1036 | 17.285 | 18.958 | −0.852 | 1.00 | 26.00 | C |
| ATOM | 252 | CB | GLU | A1036 | 17.119 | 18.801 | 0.675 | 1.00 | 24.96 | C |
| ATOM | 253 | CG | GLU | A1036 | 18.307 | 18.111 | 1.367 | 1.00 | 22.07 | C |
| ATOM | 254 | CD | GLU | A1036 | 18.254 | 18.150 | 2.900 | 1.00 | 22.03 | C |
| ATOM | 255 | OE1 | GLU | A1036 | 18.819 | 17.229 | 3.540 | 1.00 | 20.25 | O |
| ATOM | 256 | OE2 | GLU | A1036 | 17.671 | 19.102 | 3.472 | 1.00 | 19.80 | O |
| ATOM | 257 | C | GLU | A1036 | 15.934 | 19.228 | −1.526 | 1.00 | 27.44 | C |
| ATOM | 258 | O | GLU | A1036 | 15.296 | 18.314 | −2.060 | 1.00 | 26.45 | O |
| ATOM | 259 | N | MET | A1037 | 15.514 | 20.493 | −1.525 | 1.00 | 28.51 | N |
| ATOM | 260 | CA | MET | A1037 | 14.242 | 20.888 | −2.132 | 1.00 | 30.20 | C |
| ATOM | 261 | CB | MET | A1037 | 13.979 | 22.377 | −1.877 | 1.00 | 31.18 | C |
| ATOM | 262 | CG | MET | A1037 | 12.608 | 22.872 | −2.324 | 1.00 | 32.64 | C |
| ATOM | 263 | SD | MET | A1037 | 11.236 | 22.231 | −1.342 | 1.00 | 35.22 | S |
| ATOM | 264 | CE | MET | A1037 | 9.873 | 22.344 | −2.511 | 1.00 | 34.31 | C |
| ATOM | 265 | C | MET | A1037 | 14.192 | 20.608 | −3.637 | 1.00 | 30.82 | C |
| ATOM | 266 | O | MET | A1037 | 13.141 | 20.248 | −4.176 | 1.00 | 31.12 | O |
| ATOM | 267 | N | GLU | A1038 | 15.320 | 20.780 | −4.317 | 1.00 | 31.36 | N |
| ATOM | 268 | CA | GLU | A1038 | 15.371 | 20.526 | −5.756 | 1.00 | 31.95 | C |
| ATOM | 269 | CB | GLU | A1038 | 16.773 | 20.820 | −6.296 | 1.00 | 33.04 | C |
| ATOM | 270 | CG | GLU | A1038 | 17.111 | 22.300 | −6.367 | 1.00 | 35.12 | C |
| ATOM | 271 | CD | GLU | A1038 | 18.578 | 22.550 | −6.673 | 1.00 | 36.56 | C |
| ATOM | 272 | OE1 | GLU | A1038 | 18.943 | 23.713 | −6.941 | 1.00 | 37.89 | O |
| ATOM | 273 | OE2 | GLU | A1038 | 19.371 | 21.585 | −6.639 | 1.00 | 37.49 | O |
| ATOM | 274 | C | GLU | A1038 | 14.988 | 19.080 | −6.072 | 1.00 | 31.21 | C |
| ATOM | 275 | O | GLU | A1038 | 14.268 | 18.816 | −7.036 | 1.00 | 30.88 | O |
| ATOM | 276 | N | ARG | A1039 | 15.474 | 18.151 | −5.253 | 1.00 | 29.97 | N |
| ATOM | 277 | CA | ARG | A1039 | 15.188 | 16.730 | −5.427 | 1.00 | 28.84 | C |
| ATOM | 278 | CB | ARG | A1039 | 16.086 | 15.913 | −4.483 | 1.00 | 29.69 | C |
| ATOM | 279 | CG | ARG | A1039 | 15.853 | 14.402 | −4.480 | 1.00 | 31.09 | C |
| ATOM | 280 | CD | ARG | A1039 | 16.329 | 13.727 | −5.758 | 1.00 | 32.23 | C |
| ATOM | 281 | NE | ARG | A1039 | 16.125 | 12.280 | −5.708 | 1.00 | 33.37 | N |
| ATOM | 282 | CZ | ARG | A1039 | 16.076 | 11.492 | −6.780 | 1.00 | 33.30 | C |
| ATOM | 283 | NH1 | ARG | A1039 | 16.222 | 12.007 | −7.992 | 1.00 | 33.19 | N |
| ATOM | 284 | NH2 | ARG | A1039 | 15.852 | 10.191 | −6.641 | 1.00 | 32.92 | N |
| ATOM | 285 | C | ARG | A1039 | 13.709 | 16.442 | −5.145 | 1.00 | 27.71 | C |
| ATOM | 286 | O | ARG | A1039 | 13.059 | 15.705 | −5.887 | 1.00 | 26.61 | O |
| ATOM | 287 | N | PHE | A1040 | 13.189 | 17.041 | −4.076 | 1.00 | 26.75 | N |
| ATOM | 288 | CA | PHE | A1040 | 11.795 | 16.867 | −3.659 | 1.00 | 26.16 | C |
| ATOM | 289 | CB | PHE | A1040 | 11.532 | 17.729 | −2.405 | 1.00 | 25.81 | C |
| ATOM | 290 | CG | PHE | A1040 | 10.111 | 17.665 | −1.886 | 1.00 | 25.91 | C |
| ATOM | 291 | CD1 | PHE | A1040 | 9.615 | 16.508 | −1.292 | 1.00 | 25.16 | C |
| ATOM | 292 | CD2 | PHE | A1040 | 9.275 | 18.778 | −1.979 | 1.00 | 25.61 | C |
| ATOM | 293 | CE1 | PHE | A1040 | 8.309 | 16.457 | −0.798 | 1.00 | 25.37 | C |
| ATOM | 294 | CE2 | PHE | A1040 | 7.966 | 18.740 | −1.490 | 1.00 | 25.17 | C |
| ATOM | 295 | CZ | PHE | A1040 | 7.481 | 17.579 | −0.897 | 1.00 | 25.69 | C |
| ATOM | 296 | C | PHE | A1040 | 10.782 | 17.210 | −4.760 | 1.00 | 25.80 | C |
| ATOM | 297 | O | PHE | A1040 | 9.835 | 16.460 | −4.990 | 1.00 | 25.42 | O |
| ATOM | 298 | N | THR | A1041 | 10.984 | 18.332 | −5.443 | 1.00 | 25.73 | N |
| ATOM | 299 | CA | THR | A1041 | 10.053 | 18.759 | −6.493 | 1.00 | 26.24 | C |
| ATOM | 300 | CB | THR | A1041 | 10.257 | 20.259 | −6.836 | 1.00 | 26.86 | C |
| ATOM | 301 | OG1 | THR | A1041 | 11.634 | 20.507 | −7.154 | 1.00 | 26.58 | O |
| ATOM | 302 | CG2 | THR | A1041 | 9.849 | 21.130 | −5.656 | 1.00 | 26.06 | C |
| ATOM | 303 | C | THR | A1041 | 10.066 | 17.948 | −7.800 | 1.00 | 26.59 | C |
| ATOM | 304 | O | THR | A1041 | 9.149 | 18.071 | −8.620 | 1.00 | 27.01 | O |
| ATOM | 305 | N | SER | A1042 | 11.080 | 17.111 | −7.996 | 1.00 | 26.31 | N |
| ATOM | 306 | CA | SER | A1042 | 11.158 | 16.305 | −9.222 | 1.00 | 26.03 | C |
| ATOM | 307 | CB | SER | A1042 | 12.618 | 16.106 | −9.631 | 1.00 | 26.30 | C |
| ATOM | 308 | OG | SER | A1042 | 13.287 | 15.231 | −8.737 | 1.00 | 27.19 | O |
| ATOM | 309 | C | SER | A1042 | 10.488 | 14.935 | −9.082 | 1.00 | 25.97 | C |
| ATOM | 310 | O | SER | A1042 | 10.354 | 14.198 | −10.063 | 1.00 | 25.27 | O |
| ATOM | 311 | N | LEU | A1043 | 10.064 | 14.599 | −7.865 | 1.00 | 25.25 | N |
| ATOM | 312 | CA | LEU | A1043 | 9.431 | 13.310 | −7.601 | 1.00 | 25.51 | C |
| ATOM | 313 | CB | LEU | A1043 | 9.906 | 12.765 | −6.245 | 1.00 | 24.11 | C |
| ATOM | 314 | CG | LEU | A1043 | 11.425 | 12.571 | −6.118 | 1.00 | 24.06 | C |
| ATOM | 315 | CD1 | LEU | A1043 | 11.768 | 12.080 | −4.715 | 1.00 | 22.60 | C |
| ATOM | 316 | CD2 | LEU | A1043 | 11.914 | 11.571 | −7.164 | 1.00 | 23.86 | C |
| ATOM | 317 | C | LEU | A1043 | 7.907 | 13.389 | −7.634 | 1.00 | 25.82 | C |
| ATOM | 318 | O | LEU | A1043 | 7.333 | 14.475 | −7.575 | 1.00 | 26.42 | O |
| ATOM | 319 | N | LYS | A1044 | 7.256 | 12.233 | −7.723 | 1.00 | 26.67 | N |
| ATOM | 320 | CA | LYS | A1044 | 5.802 | 12.184 | −7.785 | 1.00 | 27.60 | C |
| ATOM | 321 | CB | LYS | A1044 | 5.361 | 11.803 | −9.200 | 1.00 | 29.18 | C |
| ATOM | 322 | CG | LYS | A1044 | 5.705 | 10.369 | −9.562 | 1.00 | 31.06 | C |
| ATOM | 323 | CD | LYS | A1044 | 5.359 | 10.045 | −10.999 | 1.00 | 32.81 | C |
| ATOM | 324 | CE | LYS | A1044 | 5.572 | 8.567 | −11.282 | 1.00 | 33.54 | C |
| ATOM | 325 | NZ | LYS | A1044 | 4.702 | 7.716 | −10.419 | 1.00 | 34.84 | N |
| ATOM | 326 | C | LYS | A1044 | 5.159 | 11.211 | −6.795 | 1.00 | 27.94 | C |
| ATOM | 327 | O | LYS | A1044 | 5.774 | 10.227 | −6.367 | 1.00 | 27.61 | O |
| ATOM | 328 | N | GLY | A1045 | 3.904 | 11.500 | −6.464 | 1.00 | 27.68 | N |
| ATOM | 329 | CA | GLY | A1045 | 3.124 | 10.671 | −5.562 | 1.00 | 28.74 | C |
| ATOM | 330 | C | GLY | A1045 | 3.741 | 10.224 | −4.252 | 1.00 | 28.99 | C |
| ATOM | 331 | O | GLY | A1045 | 4.282 | 11.024 | −3.487 | 1.00 | 29.00 | O |
| ATOM | 332 | N | ARG | A1046 | 3.643 | 8.925 | −3.998 | 1.00 | 29.30 | N |
| ATOM | 333 | CA | ARG | A1046 | 4.155 | 8.320 | −2.776 | 1.00 | 30.48 | C |
| ATOM | 334 | CB | ARG | A1046 | 3.790 | 6.833 | −2.754 | 1.00 | 32.57 | C |
| ATOM | 335 | CG | ARG | A1046 | 3.044 | 6.390 | −1.513 | 1.00 | 35.84 | C |
| ATOM | 336 | CD | ARG | A1046 | 2.680 | 4.922 | −1.598 | 1.00 | 38.51 | C |
| ATOM | 337 | NE | ARG | A1046 | 2.023 | 4.445 | −0.384 | 1.00 | 41.12 | N |
| ATOM | 338 | CZ | ARG | A1046 | 1.613 | 3.194 | −0.204 | 1.00 | 42.33 | C |
| ATOM | 339 | NH1 | ARG | A1046 | 1.024 | 2.842 | 0.931 | 1.00 | 42.60 | N |
| ATOM | 340 | NH2 | ARG | A1046 | 1.793 | 2.292 | −1.164 | 1.00 | 43.25 | N |
| ATOM | 341 | C | ARG | A1046 | 5.663 | 8.479 | −2.582 | 1.00 | 29.60 | C |
| ATOM | 342 | O | ARG | A1046 | 6.128 | 8.686 | −1.460 | 1.00 | 28.95 | O |
| ATOM | 343 | N | ARG | A1047 | 6.423 | 8.375 | −3.668 | 1.00 | 28.70 | N |
| ATOM | 344 | CA | ARG | A1047 | 7.875 | 8.495 | −3.585 | 1.00 | 28.01 | C |
| ATOM | 345 | CD | ARG | A1047 | 8.512 | 8.167 | −4.943 | 1.00 | 29.65 | C |
| ATOM | 346 | CG | ARG | A1047 | 9.566 | 7.069 | −4.869 | 1.00 | 32.09 | C |
| ATOM | 347 | CD | ARG | A1047 | 9.831 | 6.426 | −6.228 | 1.00 | 34.53 | C |
| ATOM | 348 | NE | ARG | A1047 | 8.601 | 5.910 | −6.825 | 1.00 | 36.57 | N |
| ATOM | 349 | CZ | ARG | A1047 | 8.548 | 5.138 | −7.906 | 1.00 | 37.35 | C |
| ATOM | 350 | NH1 | ARG | A1047 | 7.373 | 4.727 | −8.368 | 1.00 | 38.05 | N |
| ATOM | 351 | NH2 | ARG | A1047 | 9.663 | 4.767 | −8.523 | 1.00 | 38.02 | N |
| ATOM | 352 | C | ARG | A1047 | 8.288 | 9.890 | −3.117 | 1.00 | 26.86 | C |
| ATOM | 353 | O | ARG | A1047 | 9.309 | 10.048 | −2.444 | 1.00 | 25.61 | O |
| ATOM | 354 | N | GLN | A1048 | 7.481 | 10.891 | −3.464 | 1.00 | 25.04 | N |
| ATOM | 355 | CA | GLN | A1048 | 7.739 | 12.277 | −3.079 | 1.00 | 24.33 | C |
| ATOM | 356 | CB | GLN | A1048 | 6.733 | 13.217 | −3.779 | 1.00 | 25.19 | C |
| ATOM | 357 | CG | GLN | A1048 | 7.138 | 14.694 | −3.774 | 1.00 | 26.24 | C |
| ATOM | 358 | CD | GLN | A1048 | 6.221 | 15.581 | −4.620 | 1.00 | 26.41 | C |
| ATOM | 359 | OE1 | GLN | A1048 | 6.617 | 16.664 | −5.047 | 1.00 | 26.86 | O |
| ATOM | 360 | NE2 | GLN | A1048 | 4.994 | 15.127 | −4.853 | 1.00 | 27.01 | N |
| ATOM | 361 | C | GLN | A1048 | 7.632 | 12.442 | −1.553 | 1.00 | 23.25 | C |
| ATOM | 362 | O | GLN | A1048 | 8.463 | 13.110 | −0.935 | 1.00 | 23.01 | O |
| ATOM | 363 | N | ILE | A1049 | 6.615 | 11.830 | −0.949 | 1.00 | 22.49 | N |
| ATOM | 364 | CA | ILE | A1049 | 6.423 | 11.919 | 0.504 | 1.00 | 22.00 | C |
| ATOM | 365 | CB | ILE | A1049 | 5.011 | 11.414 | 0.916 | 1.00 | 23.38 | C |
| ATOM | 366 | CG2 | ILE | A1049 | 4.810 | 11.564 | 2.421 | 1.00 | 23.48 | C |
| ATOM | 367 | CG1 | ILE | A1049 | 3.928 | 12.197 | 0.161 | 1.00 | 24.39 | C |
| ATOM | 368 | CD1 | ILE | A1049 | 3.923 | 13.695 | 0.424 | 1.00 | 25.65 | C |
| ATOM | 369 | C | ILE | A1049 | 7.496 | 11.118 | 1.274 | 1.00 | 21.25 | C |
| ATOM | 370 | O | ILE | A1049 | 7.940 | 11.537 | 2.347 | 1.00 | 19.66 | O |
| ATOM | 371 | N | GLU | A1050 | 7.913 | 9.975 | 0.724 | 1.00 | 20.69 | N |
| ATOM | 372 | CA | GLU | A1050 | 8.939 | 9.146 | 1.372 | 1.00 | 20.67 | C |
| ATOM | 373 | CB | GLU | A1050 | 9.093 | 7.794 | 0.650 | 1.00 | 22.03 | C |
| ATOM | 374 | CG | GLU | A1050 | 10.362 | 7.017 | 1.051 | 1.00 | 23.69 | C |
| ATOM | 375 | CD | GLU | A1050 | 10.385 | 6.625 | 2.520 | 1.00 | 24.98 | C |
| ATOM | 376 | OE1 | GLU | A1050 | 11.498 | 6.519 | 3.095 | 1.00 | 23.91 | O |
| ATOM | 377 | OE2 | GLU | A1050 | 9.292 | 6.409 | 3.097 | 1.00 | 25.89 | O |
| ATOM | 378 | C | GLU | A1050 | 10.298 | 9.851 | 1.430 | 1.00 | 19.72 | C |
| ATOM | 379 | O | GLU | A1050 | 11.073 | 9.634 | 2.367 | 1.00 | 19.12 | O |
| ATOM | 380 | N | TYR | A1051 | 10.604 | 10.680 | 0.431 | 1.00 | 18.43 | N |
| ATOM | 381 | CA | TYR | A1051 | 11.878 | 11.400 | 0.435 | 1.00 | 17.86 | C |
| ATOM | 382 | CB | TYR | A1051 | 12.110 | 12.129 | −0.902 | 1.00 | 18.12 | C |
| ATOM | 383 | CG | TYR | A1051 | 13.379 | 12.970 | −0.932 | 1.00 | 17.88 | C |
| ATOM | 384 | CD1 | TYR | A1051 | 13.372 | 14.307 | −0.518 | 1.00 | 17.74 | C |
| ATOM | 385 | CE1 | TYR | A1051 | 14.547 | 15.076 | −0.516 | 1.00 | 17.82 | C |
| ATOM | 386 | CD2 | TYR | A1051 | 14.592 | 12.419 | −1.345 | 1.00 | 17.56 | C |
| ATOM | 387 | CE2 | TYR | A1051 | 15.770 | 13.176 | −1.344 | 1.00 | 18.21 | C |
| ATOM | 388 | CZ | TYR | A1051 | 15.740 | 14.501 | −0.929 | 1.00 | 18.16 | C |
| ATOM | 389 | OH | TYR | A1051 | 16.908 | 15.237 | −0.923 | 1.00 | 18.28 | O |
| ATOM | 390 | C | TYR | A1051 | 11.915 | 12.409 | 1.590 | 1.00 | 17.91 | C |
| ATOM | 391 | O | TYR | A1051 | 12.927 | 12.525 | 2.290 | 1.00 | 16.53 | O |
| ATOM | 392 | N | LEU | A1052 | 10.810 | 13.137 | 1.778 | 1.00 | 17.38 | N |
| ATOM | 393 | CA | LEU | A1052 | 10.709 | 14.131 | 2.851 | 1.00 | 17.07 | C |
| ATOM | 394 | CB | LEU | A1052 | 9.392 | 14.917 | 2.731 | 1.00 | 17.53 | C |
| ATOM | 395 | CG | LEU | A1052 | 8.985 | 15.844 | 3.890 | 1.00 | 17.80 | C |
| ATOM | 396 | CD1 | LEU | A1052 | 10.096 | 16.844 | 4.191 | 1.00 | 18.32 | C |
| ATOM | 397 | CD2 | LEU | A1052 | 7.693 | 16.573 | 3.524 | 1.00 | 17.67 | C |
| ATOM | 398 | C | LEU | A1052 | 10.784 | 13.448 | 4.219 | 1.00 | 16.62 | C |
| ATOM | 399 | O | LEU | A1052 | 11.544 | 13.874 | 5.093 | 1.00 | 16.61 | O |
| ATOM | 400 | N | ALA | A1053 | 10.006 | 12.383 | 4.394 | 1.00 | 15.83 | N |
| ATOM | 401 | CA | ALA | A1053 | 9.985 | 11.645 | 5.662 | 1.00 | 15.97 | C |
| ATOM | 402 | CB | ALA | A1053 | 8.974 | 10.516 | 5.587 | 1.00 | 15.80 | C |
| ATOM | 403 | C | ALA | A1053 | 11.362 | 11.091 | 6.038 | 1.00 | 15.64 | C |
| ATOM | 404 | O | ALA | A1053 | 11.750 | 11.129 | 7.208 | 1.00 | 15.53 | O |
| ATOM | 405 | N | GLY | A1054 | 12.094 | 10.573 | 5.054 | 1.00 | 14.85 | N |
| ATOM | 406 | CA | GLY | A1054 | 13.420 | 10.028 | 5.327 | 1.00 | 14.89 | C |
| ATOM | 407 | C | GLY | A1054 | 14.431 | 11.073 | 5.789 | 1.00 | 14.97 | C |
| ATOM | 408 | O | GLY | A1054 | 15.243 | 10.820 | 6.689 | 1.00 | 14.15 | O |
| ATOM | 409 | N | ARG | A1055 | 14.403 | 12.250 | 5.169 | 1.00 | 14.68 | N |
| ATOM | 410 | CA | ARG | A1055 | 15.327 | 13.313 | 5.553 | 1.00 | 15.29 | C |
| ATOM | 411 | CB | ARG | A1055 | 15.257 | 14.462 | 4.533 | 1.00 | 14.70 | C |
| ATOM | 412 | CG | ARG | A1055 | 16.364 | 14.420 | 3.438 | 1.00 | 16.14 | C |
| ATOM | 413 | CD | ARG | A1055 | 16.452 | 13.082 | 2.658 | 1.00 | 16.61 | C |
| ATOM | 414 | NE | ARG | A1055 | 17.610 | 13.039 | 1.745 | 1.00 | 16.90 | N |
| ATOM | 415 | CZ | ARG | A1055 | 17.966 | 11.976 | 1.021 | 1.00 | 17.56 | C |
| ATOM | 416 | NH1 | ARG | A1055 | 17.265 | 10.850 | 1.093 | 1.00 | 16.02 | N |
| ATOM | 417 | NH2 | ARG | A1055 | 19.028 | 12.034 | 0.215 | 1.00 | 18.06 | N |
| ATOM | 418 | C | ARG | A1055 | 15.010 | 13.804 | 6.980 | 1.00 | 15.61 | C |
| ATOM | 419 | O | ARG | A1055 | 15.909 | 14.138 | 7.755 | 1.00 | 15.05 | O |
| ATOM | 420 | N | TRP | A1056 | 13.731 | 13.826 | 7.333 | 1.00 | 15.92 | N |
| ATOM | 421 | CA | TRP | A1056 | 13.324 | 14.252 | 8.676 | 1.00 | 17.44 | C |
| ATOM | 422 | CB | TRP | A1056 | 11.791 | 14.409 | 8.706 | 1.00 | 20.42 | C |
| ATOM | 423 | CG | TRP | A1056 | 11.151 | 14.202 | 10.044 | 1.00 | 24.52 | C |
| ATOM | 424 | CD2 | TRP | A1056 | 10.650 | 15.212 | 10.921 | 1.00 | 26.47 | C |
| ATOM | 425 | CE2 | TRP | A1056 | 10.140 | 14.556 | 12.065 | 1.00 | 27.71 | C |
| ATOM | 426 | CE3 | TRP | A1056 | 10.581 | 16.607 | 10.854 | 1.00 | 27.13 | C |
| ATOM | 427 | CD1 | TRP | A1056 | 10.931 | 13.005 | 10.673 | 1.00 | 25.93 | C |
| ATOM | 428 | NE1 | TRP | A1056 | 10.325 | 13.211 | 11.885 | 1.00 | 27.77 | N |
| ATOM | 429 | CZ2 | TRP | A1056 | 9.567 | 15.250 | 13.133 | 1.00 | 28.62 | C |
| ATOM | 430 | CZ3 | TRP | A1056 | 10.012 | 17.297 | 11.915 | 1.00 | 28.11 | C |
| ATOM | 431 | CH2 | TRP | A1056 | 9.512 | 16.617 | 13.041 | 1.00 | 29.24 | C |
| ATOM | 432 | C | TRP | A1056 | 13.807 | 13.259 | 9.758 | 1.00 | 16.63 | C |
| ATOM | 433 | O | TRP | A1056 | 14.339 | 13.661 | 10.813 | 1.00 | 14.62 | O |
| ATOM | 434 | N | SER | A1057 | 13.645 | 11.965 | 9.477 | 1.00 | 15.34 | N |
| ATOM | 435 | CA | SER | A1057 | 14.034 | 10.892 | 10.404 | 1.00 | 14.89 | C |
| ATOM | 436 | CB | SER | A1057 | 13.509 | 9.549 | 9.864 | 1.00 | 16.31 | C |
| ATOM | 437 | OG | SER | A1057 | 13.653 | 8.482 | 10.792 | 1.00 | 15.29 | O |
| ATOM | 438 | C | SER | A1057 | 15.558 | 10.833 | 10.624 | 1.00 | 14.80 | C |
| ATOM | 439 | O | SER | A1057 | 16.033 | 10.618 | 11.748 | 1.00 | 13.11 | O |
| ATOM | 440 | N | ALA | A1058 | 16.326 | 11.043 | 9.560 | 1.00 | 13.86 | N |
| ATOM | 441 | CA | ALA | A1058 | 17.784 | 11.015 | 9.670 | 1.00 | 14.03 | C |
| ATOM | 442 | CB | ALA | A1058 | 18.408 | 11.077 | 8.283 | 1.00 | 14.39 | C |
| ATOM | 443 | C | ALA | A1058 | 18.326 | 12.162 | 10.528 | 1.00 | 14.15 | C |
| ATOM | 444 | O | ALA | A1058 | 19.253 | 11.972 | 11.326 | 1.00 | 12.67 | O |
| ATOM | 445 | N | LYS | A1059 | 17.760 | 13.356 | 10.346 | 1.00 | 14.25 | N |
| ATOM | 446 | CA | LYS | A1059 | 18.198 | 14.530 | 11.102 | 1.00 | 14.94 | C |
| ATOM | 447 | CB | LYS | A1059 | 17.561 | 15.807 | 10.511 | 1.00 | 14.63 | C |
| ATOM | 448 | CG | LYS | A1059 | 18.030 | 16.077 | 9.061 | 1.00 | 15.21 | C |
| ATOM | 449 | CD | LYS | A1059 | 17.605 | 17.445 | 8.497 | 1.00 | 14.78 | C |
| ATOM | 450 | CE | LYS | A1059 | 18.198 | 17.662 | 7.096 | 1.00 | 15.27 | C |
| ATOM | 451 | NZ | LYS | A1059 | 17.780 | 18.955 | 6.460 | 1.00 | 14.73 | N |
| ATOM | 452 | C | LYS | A1059 | 17.902 | 14.382 | 12.597 | 1.00 | 15.88 | C |
| ATOM | 453 | O | LYS | A1059 | 18.726 | 14.755 | 13.441 | 1.00 | 15.17 | O |
| ATOM | 454 | N | GLU | A1060 | 16.743 | 13.819 | 12.933 | 1.00 | 15.81 | N |
| ATOM | 455 | CA | GLU | A1060 | 16.395 | 13.609 | 14.337 | 1.00 | 16.66 | C |
| ATOM | 456 | CB | GLU | A1060 | 14.943 | 13.142 | 14.474 | 1.00 | 18.92 | C |
| ATOM | 457 | CG | GLU | A1060 | 13.922 | 14.183 | 14.023 | 1.00 | 23.44 | C |
| ATOM | 458 | CD | GLU | A1060 | 12.804 | 14.369 | 15.031 | 1.00 | 26.75 | C |
| ATOM | 459 | OE1 | GLU | A1060 | 12.175 | 13.358 | 15.414 | 1.00 | 29.16 | O |
| ATOM | 460 | OE2 | GLU | A1060 | 12.547 | 15.522 | 15.437 | 1.00 | 27.98 | O |
| ATOM | 461 | C | GLU | A1060 | 17.333 | 12.578 | 14.977 | 1.00 | 15.69 | C |
| ATOM | 462 | O | GLU | A1060 | 17.740 | 12.730 | 16.138 | 1.00 | 14.40 | O |
| ATOM | 463 | N | ALA | A1061 | 17.689 | 11.541 | 14.219 | 1.00 | 14.51 | N |
| ATOM | 464 | CA | ALA | A1061 | 18.586 | 10.500 | 14.727 | 1.00 | 14.88 | C |
| ATOM | 465 | CB | ALA | A1061 | 18.658 | 9.312 | 13.730 | 1.00 | 14.28 | C |
| ATOM | 466 | C | ALA | A1061 | 19.998 | 11.037 | 15.018 | 1.00 | 15.17 | C |
| ATOM | 467 | O | ALA | A1061 | 20.635 | 10.622 | 16.001 | 1.00 | 14.05 | O |
| ATOM | 468 | N | PHE | A1062 | 20.488 | 11.943 | 14.167 | 1.00 | 14.78 | N |
| ATOM | 469 | CA | PHE | A1062 | 21.811 | 12.545 | 14.365 | 1.00 | 16.06 | C |
| ATOM | 470 | CB | PHE | A1062 | 22.200 | 13.433 | 13.161 | 1.00 | 16.25 | C |
| ATOM | 471 | CG | PHE | A1062 | 23.514 | 14.176 | 13.341 | 1.00 | 17.02 | C |
| ATOM | 472 | CD1 | PHE | A1062 | 23.562 | 15.384 | 14.039 | 1.00 | 17.84 | C |
| ATOM | 473 | CD2 | PHE | A1062 | 24.707 | 13.642 | 12.847 | 1.00 | 16.96 | C |
| ATOM | 474 | CE1 | PHE | A1062 | 24.783 | 16.049 | 14.247 | 1.00 | 17.77 | C |
| ATOM | 475 | CE2 | PHE | A1062 | 25.931 | 14.297 | 13.049 | 1.00 | 17.84 | C |
| ATOM | 476 | CZ | PHE | A1062 | 25.964 | 15.505 | 13.754 | 1.00 | 18.67 | C |
| ATOM | 477 | C | PHE | A1062 | 21.830 | 13.380 | 15.651 | 1.00 | 16.47 | C |
| ATOM | 478 | O | PHE | A1062 | 22.792 | 13.326 | 16.422 | 1.00 | 15.43 | O |
| ATOM | 479 | N | SER | A1063 | 20.760 | 14.138 | 15.895 | 1.00 | 17.69 | N |
| ATOM | 480 | CA | SER | A1063 | 20.695 | 14.979 | 17.093 | 1.00 | 19.00 | C |
| ATOM | 481 | CB | SER | A1063 | 19.446 | 15.881 | 17.067 | 1.00 | 20.22 | C |
| ATOM | 482 | OG | SER | A1063 | 18.276 | 15.162 | 17.434 | 1.00 | 23.89 | O |
| ATOM | 483 | C | SER | A1063 | 20.707 | 14.148 | 18.383 | 1.00 | 19.17 | C |
| ATOM | 484 | O | SER | A1063 | 21.229 | 14.602 | 19.405 | 1.00 | 19.65 | O |
| ATOM | 485 | N | LYS | A1064 | 20.124 | 12.949 | 18.353 | 1.00 | 18.93 | N |
| ATOM | 486 | CA | LYS | A1064 | 20.121 | 12.093 | 19.545 | 1.00 | 19.27 | C |
| ATOM | 487 | CB | LYS | A1064 | 19.094 | 10.961 | 19.418 | 1.00 | 19.83 | C |
| ATOM | 488 | CG | LYS | A1064 | 17.648 | 11.413 | 19.471 | 1.00 | 21.72 | C |
| ATOM | 489 | CD | LYS | A1064 | 16.720 | 10.201 | 19.456 | 1.00 | 23.02 | C |
| ATOM | 490 | CE | LYS | A1064 | 15.270 | 10.614 | 19.240 | 1.00 | 24.30 | C |
| ATOM | 491 | NZ | LYS | A1064 | 14.379 | 9.425 | 19.074 | 1.00 | 25.01 | N |
| ATOM | 492 | C | LYS | A1064 | 21.510 | 11.497 | 19.770 | 1.00 | 19.33 | C |
| ATOM | 493 | O | LYS | A1064 | 21.920 | 11.256 | 20.922 | 1.00 | 18.44 | O |
| ATOM | 494 | N | ALA | A1065 | 22.230 | 11.254 | 18.674 | 1.00 | 18.91 | N |
| ATOM | 495 | CA | ALA | A1065 | 23.588 | 10.716 | 18.757 | 1.00 | 20.48 | C |
| ATOM | 496 | CB | ALA | A1065 | 24.110 | 10.348 | 17.359 | 1.00 | 19.55 | C |
| ATOM | 497 | C | ALA | A1065 | 24.515 | 11.745 | 19.417 | 1.00 | 22.05 | C |
| ATOM | 498 | O | ALA | A1065 | 25.486 | 11.374 | 20.086 | 1.00 | 22.04 | O |
| ATOM | 499 | N | MET | A1066 | 24.224 | 13.032 | 19.224 | 1.00 | 23.48 | N |
| ATOM | 500 | CA | MET | A1066 | 25.027 | 14.101 | 19.836 | 1.00 | 26.32 | C |
| ATOM | 501 | CB | MET | A1066 | 24.893 | 15.409 | 19.044 | 1.00 | 27.55 | C |
| ATOM | 502 | CG | MET | A1066 | 25.471 | 15.377 | 17.634 | 1.00 | 29.85 | C |
| ATOM | 503 | SD | MET | A1066 | 27.227 | 15.839 | 17.541 | 1.00 | 34.49 | S |
| ATOM | 504 | CE | MET | A1066 | 27.100 | 17.585 | 17.194 | 1.00 | 32.80 | C |
| ATOM | 505 | C | MET | A1066 | 24.554 | 14.334 | 21.277 | 1.00 | 27.22 | C |
| ATOM | 506 | O | MET | A1066 | 25.272 | 14.916 | 22.093 | 1.00 | 27.02 | O |
| ATOM | 507 | N | GLY | A1067 | 23.335 | 13.885 | 21.572 | 1.00 | 28.21 | N |
| ATOM | 508 | CA | GLY | A1067 | 22.770 | 14.032 | 22.905 | 1.00 | 29.77 | C |
| ATOM | 509 | C | GLY | A1067 | 22.161 | 15.395 | 23.194 | 1.00 | 31.30 | C |
| ATOM | 510 | O | GLY | A1067 | 21.943 | 15.746 | 24.356 | 1.00 | 30.88 | O |
| ATOM | 511 | N | THR | A1068 | 21.857 | 16.153 | 22.145 | 1.00 | 32.65 | N |
| ATOM | 512 | CA | THR | A1068 | 21.305 | 17.495 | 22.309 | 1.00 | 34.08 | C |
| ATOM | 513 | CB | THR | A1068 | 22.128 | 18.520 | 21.510 | 1.00 | 34.54 | C |
| ATOM | 514 | OG1 | THR | A1068 | 21.950 | 18.280 | 20.109 | 1.00 | 34.50 | O |
| ATOM | 515 | CG2 | THR | A1068 | 23.613 | 18.400 | 21.849 | 1.00 | 34.68 | C |
| ATOM | 516 | C | THR | A1068 | 19.843 | 17.694 | 21.911 | 1.00 | 35.08 | C |
| ATOM | 517 | O | THR | A1068 | 19.199 | 18.637 | 22.380 | 1.00 | 35.34 | O |
| ATOM | 518 | N | GLY | A1069 | 19.320 | 16.834 | 21.046 | 1.00 | 35.84 | N |
| ATOM | 519 | CA | GLY | A1069 | 17.942 | 16.995 | 20.611 | 1.00 | 37.37 | C |
| ATOM | 520 | C | GLY | A1069 | 17.855 | 18.092 | 19.561 | 1.00 | 38.51 | C |
| ATOM | 521 | O | GLY | A1069 | 18.739 | 18.946 | 19.478 | 1.00 | 38.69 | O |
| ATOM | 522 | N | ILE | A1070 | 16.801 | 18.088 | 18.752 | 1.00 | 39.34 | N |
| ATOM | 523 | CA | ILE | A1070 | 16.688 | 19.108 | 17.722 | 1.00 | 40.39 | C |
| ATOM | 524 | CB | ILE | A1070 | 16.453 | 18.482 | 16.329 | 1.00 | 40.56 | C |
| ATOM | 525 | CG2 | ILE | A1070 | 15.019 | 17.992 | 16.207 | 1.00 | 40.43 | C |
| ATOM | 526 | CG1 | ILE | A1070 | 16.784 | 19.515 | 15.245 | 1.00 | 40.90 | C |
| ATOM | 527 | CD1 | ILE | A1070 | 16.900 | 18.947 | 13.841 | 1.00 | 40.92 | C |
| ATOM | 528 | C | ILE | A1070 | 15.616 | 20.147 | 18.019 | 1.00 | 41.07 | C |
| ATOM | 529 | O | ILE | A1070 | 14.461 | 19.826 | 18.305 | 1.00 | 41.30 | O |
| ATOM | 530 | N | SER | A1071 | 16.046 | 21.398 | 17.936 | 1.00 | 41.96 | N |
| ATOM | 531 | CA | SER | A1071 | 15.247 | 22.588 | 18.201 | 1.00 | 42.87 | C |
| ATOM | 532 | CB | SER | A1071 | 14.184 | 22.325 | 19.271 | 1.00 | 43.28 | C |
| ATOM | 533 | OG | SER | A1071 | 14.779 | 21.937 | 20.496 | 1.00 | 44.51 | O |
| ATOM | 534 | C | SER | A1071 | 16.314 | 23.521 | 18.753 | 1.00 | 43.09 | C |
| ATOM | 535 | O | SER | A1071 | 16.254 | 24.741 | 18.585 | 1.00 | 43.47 | O |
| ATOM | 536 | N | LYS | A1072 | 17.295 | 22.914 | 19.419 | 1.00 | 42.86 | N |
| ATOM | 537 | CA | LYS | A1072 | 18.427 | 23.637 | 19.982 | 1.00 | 42.57 | C |
| ATOM | 538 | CB | LYS | A1072 | 19.143 | 22.768 | 21.022 | 1.00 | 42.32 | C |
| ATOM | 539 | CG | LYS | A1072 | 18.214 | 22.218 | 22.093 | 0.05 | 42.40 | C |
| ATOM | 540 | CD | LYS | A1072 | 17.501 | 23.339 | 22.829 | 0.05 | 42.37 | C |
| ATOM | 541 | CE | LYS | A1072 | 16.446 | 22.789 | 23.773 | 0.05 | 42.36 | C |
| ATOM | 542 | NZ | LYS | A1072 | 15.708 | 23.878 | 24.472 | 0.05 | 42.34 | N |
| ATOM | 543 | C | LYS | A1072 | 19.330 | 23.904 | 18.783 | 1.00 | 42.27 | C |
| ATOM | 544 | O | LYS | A1072 | 20.222 | 24.752 | 18.814 | 1.00 | 42.17 | O |
| ATOM | 545 | N | LEU | A1073 | 19.065 | 23.149 | 17.723 | 1.00 | 41.69 | N |
| ATOM | 546 | CA | LEU | A1073 | 19.773 | 23.255 | 16.458 | 1.00 | 41.03 | C |
| ATOM | 547 | CB | LEU | A1073 | 20.879 | 22.197 | 16.365 | 1.00 | 41.27 | C |
| ATOM | 548 | CG | LEU | A1073 | 21.578 | 22.058 | 15.008 | 1.00 | 41.68 | C |
| ATOM | 549 | CD1 | LEU | A1073 | 23.024 | 21.623 | 15.193 | 1.00 | 41.49 | C |
| ATOM | 550 | CD2 | LEU | A1073 | 20.819 | 21.060 | 14.156 | 1.00 | 41.59 | C |
| ATOM | 551 | C | LEU | A1073 | 18.696 | 23.006 | 15.408 | 1.00 | 39.74 | C |
| ATOM | 552 | O | LEU | A1073 | 17.999 | 21.994 | 15.458 | 1.00 | 40.51 | O |
| ATOM | 553 | N | GLY | A1074 | 18.550 | 23.937 | 14.473 | 1.00 | 37.90 | N |
| ATOM | 554 | CA | GLY | A1074 | 17.532 | 23.795 | 13.452 | 1.00 | 34.96 | C |
| ATOM | 555 | C | GLY | A1074 | 17.853 | 22.756 | 12.399 | 1.00 | 32.76 | C |
| ATOM | 556 | O | GLY | A1074 | 18.968 | 22.237 | 12.331 | 1.00 | 31.86 | O |
| ATOM | 557 | N | PHE | A1075 | 16.861 | 22.447 | 11.575 | 1.00 | 30.76 | N |
| ATOM | 558 | CA | PHE | A1075 | 17.047 | 21.474 | 10.510 | 1.00 | 29.13 | C |
| ATOM | 559 | CB | PHE | A1075 | 15.691 | 21.061 | 9.913 | 1.00 | 28.59 | C |
| ATOM | 560 | CG | PHE | A1075 | 14.795 | 20.317 | 10.871 | 1.00 | 28.14 | C |
| ATOM | 561 | CD1 | PHE | A1075 | 14.129 | 20.990 | 11.893 | 1.00 | 28.80 | C |
| ATOM | 562 | CD2 | PHE | A1075 | 14.617 | 18.940 | 10.747 | 1.00 | 27.48 | C |
| ATOM | 563 | CE1 | PHE | A1075 | 13.294 | 20.301 | 12.780 | 1.00 | 28.53 | C |
| ATOM | 564 | CE2 | PHE | A1075 | 13.787 | 18.239 | 11.624 | 1.00 | 28.38 | C |
| ATOM | 565 | CZ | PHE | A1075 | 13.123 | 18.921 | 12.644 | 1.00 | 28.31 | C |
| ATOM | 566 | C | PHE | A1075 | 17.940 | 22.039 | 9.402 | 1.00 | 27.80 | C |
| ATOM | 567 | O | PHE | A1075 | 18.556 | 21.280 | 8.655 | 1.00 | 27.44 | O |
| ATOM | 568 | N | GLN | A1076 | 18.022 | 23.366 | 9.301 | 1.00 | 26.94 | N |
| ATOM | 569 | CA | GLN | A1076 | 18.828 | 24.003 | 8.250 | 1.00 | 26.57 | C |
| ATOM | 570 | CB | GLN | A1076 | 18.468 | 25.490 | 8.111 | 1.00 | 27.23 | C |
| ATOM | 571 | CG | GLN | A1076 | 17.037 | 25.783 | 7.655 | 1.00 | 28.06 | C |
| ATOM | 572 | CD | GLN | A1076 | 16.725 | 25.253 | 6.261 | 1.00 | 28.68 | C |
| ATOM | 573 | OE1 | GLN | A1076 | 17.504 | 25.432 | 5.321 | 1.00 | 28.59 | O |
| ATOM | 574 | NE2 | GLN | A1076 | 15.571 | 24.616 | 6.119 | 1.00 | 28.78 | N |
| ATOM | 575 | C | GLN | A1076 | 20.341 | 23.880 | 8.435 | 1.00 | 26.18 | C |
| ATOM | 576 | O | GLN | A1076 | 21.110 | 24.292 | 7.565 | 1.00 | 25.59 | O |
| ATOM | 577 | N | ASP | A1077 | 20.774 | 23.318 | 9.558 | 1.00 | 25.77 | N |
| ATOM | 578 | CA | ASP | A1077 | 22.204 | 23.158 | 9.808 | 1.00 | 25.68 | C |
| ATOM | 579 | CB | ASP | A1077 | 22.498 | 23.399 | 11.287 | 1.00 | 28.73 | C |
| ATOM | 580 | CG | ASP | A1077 | 23.952 | 23.697 | 11.542 | 1.00 | 30.84 | C |
| ATOM | 581 | OD1 | ASP | A1077 | 24.457 | 24.688 | 10.969 | 1.00 | 33.09 | O |
| ATOM | 582 | OD2 | ASP | A1077 | 24.590 | 22.947 | 12.309 | 1.00 | 32.53 | O |
| ATOM | 583 | C | ASP | A1077 | 22.729 | 21.770 | 9.393 | 1.00 | 24.42 | C |
| ATOM | 584 | O | ASP | A1077 | 23.928 | 21.487 | 9.516 | 1.00 | 23.80 | O |
| ATOM | 585 | N | LEU | A1078 | 21.831 | 20.919 | 8.899 | 1.00 | 22.23 | N |
| ATOM | 586 | CA | LEU | A1078 | 22.187 | 19.559 | 8.463 | 1.00 | 20.68 | C |
| ATOM | 587 | CB | LEU | A1078 | 21.530 | 18.525 | 9.398 | 1.00 | 19.99 | C |
| ATOM | 588 | CG | LEU | A1078 | 21.781 | 18.646 | 10.911 | 1.00 | 19.68 | C |
| ATOM | 589 | CD1 | LEU | A1078 | 20.853 | 17.703 | 11.686 | 1.00 | 19.87 | C |
| ATOM | 590 | CD2 | LEU | A1078 | 23.241 | 18.328 | 11.221 | 1.00 | 20.17 | C |
| ATOM | 591 | C | LEU | A1078 | 21.706 | 19.326 | 7.022 | 1.00 | 20.16 | C |
| ATOM | 592 | O | LEU | A1078 | 20.708 | 19.915 | 6.605 | 1.00 | 19.88 | O |
| ATOM | 593 | N | GLU | A1079 | 22.408 | 18.474 | 6.265 | 1.00 | 19.89 | N |
| ATOM | 594 | CA | GLU | A1079 | 22.021 | 18.165 | 4.875 | 1.00 | 18.83 | C |
| ATOM | 595 | CB | GLU | A1079 | 22.690 | 19.153 | 3.905 | 1.00 | 19.73 | C |
| ATOM | 596 | CG | GLU | A1079 | 22.172 | 19.085 | 2.459 | 1.00 | 21.03 | C |
| ATOM | 597 | CD | GLU | A1079 | 22.914 | 20.028 | 1.510 | 1.00 | 22.47 | C |
| ATOM | 598 | OE1 | GLU | A1079 | 24.127 | 19.815 | 1.271 | 1.00 | 22.27 | O |
| ATOM | 599 | OE2 | GLU | A1079 | 22.279 | 20.982 | 1.002 | 1.00 | 23.12 | O |
| ATOM | 600 | C | GLU | A1079 | 22.385 | 16.716 | 4.484 | 1.00 | 18.06 | C |
| ATOM | 601 | O | GLU | A1079 | 23.492 | 16.242 | 4.778 | 1.00 | 17.37 | O |
| ATOM | 602 | N | VAL | A1080 | 21.451 | 16.023 | 3.826 | 1.00 | 16.74 | N |
| ATOM | 603 | CA | VAL | A1080 | 21.644 | 14.626 | 3.396 | 1.00 | 16.37 | C |
| ATOM | 604 | CB | VAL | A1080 | 20.633 | 13.675 | 4.138 | 1.00 | 16.24 | C |
| ATOM | 605 | CG1 | VAL | A1080 | 20.859 | 12.217 | 3.738 | 1.00 | 16.31 | C |
| ATOM | 606 | CG2 | VAL | A1080 | 20.776 | 13.832 | 5.654 | 1.00 | 16.73 | C |
| ATOM | 607 | C | VAL | A1080 | 21.454 | 14.439 | 1.868 | 1.00 | 16.11 | C |
| ATOM | 608 | O | VAL | A1080 | 20.356 | 14.635 | 1.357 | 1.00 | 16.59 | O |
| ATOM | 609 | N | LEU | A1081 | 22.519 | 14.060 | 1.155 | 1.00 | 16.23 | N |
| ATOM | 610 | CA | LEU | A1081 | 22.473 | 13.830 | −0.303 | 1.00 | 16.11 | C |
| ATOM | 611 | CB | LEU | A1081 | 23.488 | 14.749 | −1.017 | 1.00 | 16.03 | C |
| ATOM | 612 | CG | LEU | A1081 | 23.400 | 16.265 | −0.767 | 1.00 | 17.13 | C |
| ATOM | 613 | CD1 | LEU | A1081 | 24.565 | 16.983 | −1.464 | 1.00 | 16.85 | C |
| ATOM | 614 | CD2 | LEU | A1081 | 22.064 | 16.798 | −1.283 | 1.00 | 17.61 | C |
| ATOM | 615 | C | LEU | A1081 | 22.790 | 12.350 | −0.624 | 1.00 | 16.47 | C |
| ATOM | 616 | O | LEU | A1081 | 22.881 | 11.530 | 0.285 | 1.00 | 15.85 | O |
| ATOM | 617 | N | ASN | A1082 | 22.954 | 12.004 | −1.906 | 1.00 | 17.23 | N |
| ATOM | 618 | CA | ASN | A1082 | 23.266 | 10.615 | −2.299 | 1.00 | 18.16 | C |
| ATOM | 619 | CB | ASN | A1082 | 22.169 | 10.064 | −3.227 | 1.00 | 19.47 | C |
| ATOM | 620 | CG | ASN | A1082 | 20.853 | 9.800 | −2.496 | 1.00 | 20.37 | C |
| ATOM | 621 | OD1 | ASN | A1082 | 20.701 | 8.793 | −1.806 | 1.00 | 23.12 | O |
| ATOM | 622 | ND2 | ASN | A1082 | 19.908 | 10.709 | −2.637 | 1.00 | 21.16 | N |
| ATOM | 623 | C | ASN | A1082 | 24.633 | 10.540 | −3.004 | 1.00 | 18.65 | C |
| ATOM | 624 | O | ASN | A1082 | 24.894 | 11.311 | −3.936 | 1.00 | 18.53 | O |
| ATOM | 625 | N | ASN | A1083 | 25.501 | 9.621 | −2.567 | 1.00 | 18.47 | N |
| ATOM | 626 | CA | ASN | A1083 | 26.840 | 9.496 | −3.160 | 1.00 | 19.58 | C |
| ATOM | 627 | CB | ASN | A1083 | 27.823 | 8.831 | −2.172 | 1.00 | 19.14 | C |
| ATOM | 628 | CG | ASN | A1083 | 27.525 | 7.354 | −1.898 | 1.00 | 19.47 | C |
| ATOM | 629 | OD1 | ASN | A1083 | 28.000 | 6.806 | −0.896 | 1.00 | 21.52 | O |
| ATOM | 630 | ND2 | ASN | A1083 | 26.774 | 6.705 | −2.778 | 1.00 | 18.08 | N |
| ATOM | 631 | C | ASN | A1083 | 26.879 | 8.801 | −4.522 | 1.00 | 21.02 | C |
| ATOM | 632 | O | ASN | A1083 | 25.835 | 8.408 | −5.053 | 1.00 | 20.46 | O |
| ATOM | 633 | N | GLU | A1084 | 28.073 | 8.655 | −5.095 | 1.00 | 22.56 | N |
| ATOM | 634 | CA | GLU | A1084 | 28.183 | 8.048 | −6.422 | 1.00 | 25.48 | C |
| ATOM | 635 | CB | GLU | A1084 | 29.606 | 8.243 | −6.993 | 1.00 | 26.01 | C |
| ATOM | 636 | CG | GLU | A1084 | 30.704 | 7.346 | −6.452 | 1.00 | 27.48 | C |
| ATOM | 637 | CD | GLU | A1084 | 32.055 | 7.621 | −7.127 | 1.00 | 28.81 | C |
| ATOM | 638 | OE1 | GLU | A1084 | 32.087 | 7.807 | −8.365 | 1.00 | 28.44 | O |
| ATOM | 639 | OE2 | GLU | A1084 | 33.085 | 7.644 | −6.424 | 1.00 | 29.66 | O |
| ATOM | 640 | C | GLU | A1084 | 27.747 | 6.583 | −6.523 | 1.00 | 26.10 | C |
| ATOM | 641 | O | GLU | A1084 | 27.675 | 6.030 | −7.618 | 1.00 | 26.96 | O |
| ATOM | 642 | N | ARG | A1085 | 27.446 | 5.958 | −5.390 | 1.00 | 26.87 | N |
| ATOM | 643 | CA | ARG | A1085 | 26.976 | 4.571 | −5.381 | 1.00 | 27.01 | C |
| ATOM | 644 | CB | ARG | A1085 | 27.697 | 3.761 | −4.302 | 1.00 | 29.89 | C |
| ATOM | 645 | CG | ARG | A1085 | 29.210 | 3.670 | −4.479 | 1.00 | 33.27 | C |
| ATOM | 646 | CD | ARG | A1085 | 29.603 | 2.687 | −5.570 | 1.00 | 35.91 | C |
| ATOM | 647 | NE | ARG | A1085 | 31.041 | 2.724 | −5.834 | 1.00 | 37.82 | N |
| ATOM | 648 | CZ | ARG | A1085 | 31.696 | 1.814 | −6.549 | 1.00 | 39.02 | C |
| ATOM | 649 | NH1 | ARG | A1085 | 33.005 | 1.929 | −6.739 | 1.00 | 39.21 | N |
| ATOM | 650 | NH2 | ARG | A1085 | 31.043 | 0.782 | −7.067 | 1.00 | 39.98 | N |
| ATOM | 651 | C | ARG | A1085 | 25.467 | 4.573 | −5.097 | 1.00 | 26.06 | C |
| ATOM | 652 | O | ARG | A1085 | 24.830 | 3.515 | −5.033 | 1.00 | 26.15 | O |
| ATOM | 653 | N | GLY | A1086 | 24.911 | 5.768 | −4.907 | 1.00 | 24.38 | N |
| ATOM | 654 | CA | GLY | A1086 | 23.485 | 5.910 | −4.649 | 1.00 | 22.84 | C |
| ATOM | 655 | C | GLY | A1086 | 22.989 | 5.874 | −3.207 | 1.00 | 21.70 | C |
| ATOM | 656 | O | GLY | A1086 | 21.780 | 5.910 | −2.980 | 1.00 | 21.52 | O |
| ATOM | 657 | N | ALA | A1087 | 23.896 | 5.813 | −2.234 | 1.00 | 20.27 | N |
| ATOM | 658 | CA | ALA | A1087 | 23.506 | 5.757 | −0.819 | 1.00 | 19.12 | C |
| ATOM | 659 | CB | ALA | A1087 | 24.452 | 4.825 | −0.065 | 1.00 | 19.31 | C |
| ATOM | 660 | C | ALA | A1087 | 23.468 | 7.126 | −0.123 | 1.00 | 18.28 | C |
| ATOM | 661 | O | ALA | A1087 | 24.293 | 8.001 | −0.400 | 1.00 | 17.46 | O |
| ATOM | 662 | N | PRO | A1088 | 22.511 | 7.322 | 0.807 | 1.00 | 17.16 | N |
| ATOM | 663 | CD | PRO | A1088 | 21.424 | 6.400 | 1.186 | 1.00 | 17.79 | C |
| ATOM | 664 | CA | PRO | A1088 | 22.398 | 8.598 | 1.529 | 1.00 | 16.21 | C |
| ATOM | 665 | CB | PRO | A1088 | 21.053 | 8.469 | 2.263 | 1.00 | 16.47 | C |
| ATOM | 666 | CG | PRO | A1088 | 20.938 | 6.995 | 2.507 | 1.00 | 17.39 | C |
| ATOM | 667 | C | PRO | A1088 | 23.579 | 8.828 | 2.481 | 1.00 | 15.55 | C |
| ATOM | 668 | O | PRO | A1088 | 24.084 | 7.876 | 3.082 | 1.00 | 14.47 | O |
| ATOM | 669 | N | TYR | A1089 | 24.018 | 10.085 | 2.597 | 1.00 | 14.68 | N |
| ATOM | 670 | CA | TYR | A1089 | 25.148 | 10.453 | 3.466 | 1.00 | 15.17 | C |
| ATOM | 671 | CB | TYR | A1089 | 26.478 | 10.205 | 2.728 | 1.00 | 15.96 | C |
| ATOM | 672 | CG | TYR | A1089 | 26.834 | 11.280 | 1.707 | 1.00 | 17.14 | C |
| ATOM | 673 | CD1 | TYR | A1089 | 27.815 | 12.240 | 1.980 | 1.00 | 17.14 | C |
| ATOM | 674 | CE1 | TYR | A1089 | 28.136 | 13.243 | 1.042 | 1.00 | 17.71 | C |
| ATOM | 675 | CD2 | TYR | A1089 | 26.180 | 11.345 | 0.476 | 1.00 | 17.70 | C |
| ATOM | 676 | CE2 | TYR | A1089 | 26.490 | 12.342 | −0.470 | 1.00 | 18.23 | C |
| ATOM | 677 | CZ | TYR | A1089 | 27.467 | 13.286 | −0.180 | 1.00 | 18.79 | C |
| ATOM | 678 | OH | TYR | A1089 | 27.769 | 14.272 | −1.110 | 1.00 | 18.83 | O |
| ATOM | 679 | C | TYR | A1089 | 25.068 | 11.931 | 3.886 | 1.00 | 15.23 | C |
| ATOM | 680 | O | TYR | A1089 | 24.480 | 12.749 | 3.172 | 1.00 | 14.55 | O |
| ATOM | 681 | N | PHE | A1090 | 25.664 | 12.276 | 5.030 | 1.00 | 15.03 | N |
| ATOM | 682 | CA | PHE | A1090 | 25.646 | 13.668 | 5.502 | 1.00 | 16.07 | C |
| ATOM | 683 | CB | PHE | A1090 | 25.891 | 13.743 | 7.023 | 1.00 | 15.87 | C |
| ATOM | 684 | CG | PHE | A1090 | 24.662 | 13.469 | 7.864 | 1.00 | 15.54 | C |
| ATOM | 685 | CD1 | PHE | A1090 | 24.359 | 12.176 | 8.287 | 1.00 | 14.81 | C |
| ATOM | 686 | CD2 | PHE | A1090 | 23.798 | 14.512 | 8.213 | 1.00 | 15.28 | C |
| ATOM | 687 | CE1 | PHE | A1090 | 23.207 | 11.920 | 9.048 | 1.00 | 15.40 | C |
| ATOM | 688 | CE2 | PHE | A1090 | 22.643 | 14.272 | 8.973 | 1.00 | 16.17 | C |
| ATOM | 689 | CZ | PHE | A1090 | 22.347 | 12.971 | 9.390 | 1.00 | 15.25 | C |
| ATOM | 690 | C | PHE | A1090 | 26.694 | 14.553 | 4.800 | 1.00 | 16.94 | C |
| ATOM | 691 | O | PHE | A1090 | 27.898 | 14.378 | 5.005 | 1.00 | 15.67 | O |
| ATOM | 692 | N | SER | A1091 | 26.233 | 15.499 | 3.983 | 1.00 | 18.05 | N |
| ATOM | 693 | CA | SER | A1091 | 27.135 | 16.421 | 3.285 | 1.00 | 20.04 | C |
| ATOM | 694 | CB | SER | A1091 | 26.528 | 16.864 | 1.949 | 1.00 | 20.26 | C |
| ATOM | 695 | OG | SER | A1091 | 25.196 | 17.305 | 2.106 | 1.00 | 21.03 | O |
| ATOM | 696 | C | SER | A1091 | 27.456 | 17.653 | 4.143 | 1.00 | 20.79 | C |
| ATOM | 697 | O | SER | A1091 | 28.445 | 18.349 | 3.890 | 1.00 | 21.71 | O |
| ATOM | 698 | N | GLN | A1092 | 26.606 | 17.930 | 5.133 | 1.00 | 20.94 | N |
| ATOM | 699 | CA | GLN | A1092 | 26.810 | 19.049 | 6.062 | 1.00 | 21.39 | C |
| ATOM | 700 | CB | GLN | A1092 | 25.958 | 20.265 | 5.665 | 1.00 | 23.27 | C |
| ATOM | 701 | CG | GLN | A1092 | 26.267 | 20.898 | 4.305 | 1.00 | 25.76 | C |
| ATOM | 702 | CD | GLN | A1092 | 27.623 | 21.589 | 4.244 | 1.00 | 27.71 | C |
| ATOM | 703 | OE1 | GLN | A1092 | 28.044 | 22.248 | 5.197 | 1.00 | 29.37 | O |
| ATOM | 704 | NE2 | GLN | A1092 | 28.301 | 21.461 | 3.106 | 1.00 | 28.25 | N |
| ATOM | 705 | C | GLN | A1092 | 26.416 | 18.619 | 7.489 | 1.00 | 20.95 | C |
| ATOM | 706 | O | GLN | A1092 | 25.340 | 18.064 | 7.689 | 1.00 | 20.12 | O |
| ATOM | 707 | N | ALA | A1093 | 27.290 | 18.868 | 8.466 | 1.00 | 20.33 | N |
| ATOM | 708 | CA | ALA | A1093 | 27.030 | 18.533 | 9.874 | 1.00 | 20.84 | C |
| ATOM | 709 | CB | ALA | A1093 | 26.874 | 17.013 | 10.046 | 1.00 | 20.16 | C |
| ATOM | 710 | C | ALA | A1093 | 28.179 | 19.041 | 10.757 | 1.00 | 21.48 | C |
| ATOM | 711 | O | ALA | A1093 | 29.319 | 19.128 | 10.304 | 1.00 | 21.44 | O |
| ATOM | 712 | N | PRO | A1094 | 27.890 | 19.389 | 12.027 | 1.00 | 22.28 | N |
| ATOM | 713 | CD | PRO | A1094 | 26.539 | 19.598 | 12.585 | 1.00 | 22.57 | C |
| ATOM | 714 | CA | PRO | A1094 | 28.921 | 19.887 | 12.954 | 1.00 | 22.63 | C |
| ATOM | 715 | CB | PRO | A1094 | 28.125 | 20.803 | 13.876 | 1.00 | 22.88 | C |
| ATOM | 716 | CG | PRO | A1094 | 26.828 | 20.043 | 14.020 | 1.00 | 23.37 | C |
| ATOM | 717 | C | PRO | A1094 | 29.678 | 18.791 | 13.731 | 1.00 | 23.05 | C |
| ATOM | 718 | O | PRO | A1094 | 29.571 | 18.700 | 14.959 | 1.00 | 23.97 | O |
| ATOM | 719 | N | PHE | A1095 | 30.446 | 17.976 | 13.009 | 1.00 | 22.89 | N |
| ATOM | 720 | CA | PHE | A1095 | 31.232 | 16.883 | 13.590 | 1.00 | 22.99 | C |
| ATOM | 721 | CB | PHE | A1095 | 30.341 | 15.639 | 13.767 | 1.00 | 22.67 | C |
| ATOM | 722 | CG | PHE | A1095 | 31.067 | 14.436 | 14.307 | 1.00 | 22.55 | C |
| ATOM | 723 | CD1 | PHE | A1095 | 31.374 | 13.360 | 13.474 | 1.00 | 22.34 | C |
| ATOM | 724 | CD2 | PHE | A1095 | 31.462 | 14.385 | 15.641 | 1.00 | 22.13 | C |
| ATOM | 725 | CE1 | PHE | A1095 | 32.068 | 12.250 | 13.963 | 1.00 | 22.27 | C |
| ATOM | 726 | CE2 | PHE | A1095 | 32.156 | 13.281 | 16.143 | 1.00 | 22.71 | C |
| ATOM | 727 | CZ | PHE | A1095 | 32.461 | 12.209 | 15.300 | 1.00 | 22.51 | C |
| ATOM | 728 | C | PHE | A1095 | 32.406 | 16.592 | 12.639 | 1.00 | 23.22 | C |
| ATOM | 729 | O | PHE | A1095 | 32.221 | 16.574 | 11.426 | 1.00 | 23.38 | O |
| ATOM | 730 | N | SER | A1096 | 33.602 | 16.353 | 13.183 | 1.00 | 23.37 | N |
| ATOM | 731 | CA | SER | A1096 | 34.792 | 16.122 | 12.347 | 1.00 | 23.62 | C |
| ATOM | 732 | CB | SER | A1096 | 35.993 | 16.858 | 12.955 | 1.00 | 24.11 | C |
| ATOM | 733 | OG | SER | A1096 | 36.331 | 16.317 | 14.223 | 1.00 | 26.37 | O |
| ATOM | 734 | C | SER | A1096 | 35.233 | 14.691 | 12.000 | 1.00 | 23.23 | C |
| ATOM | 735 | O | SER | A1096 | 36.094 | 14.510 | 11.125 | 1.00 | 22.88 | O |
| ATOM | 736 | N | GLY | A1097 | 34.670 | 13.686 | 12.669 | 1.00 | 22.73 | N |
| ATOM | 737 | CA | GLY | A1097 | 35.043 | 12.304 | 12.391 | 1.00 | 21.96 | C |
| ATOM | 738 | C | GLY | A1097 | 34.138 | 11.589 | 11.393 | 1.00 | 21.41 | C |
| ATOM | 739 | O | GLY | A1097 | 33.518 | 12.232 | 10.555 | 1.00 | 21.73 | O |
| ATOM | 740 | N | LYS | A1098 | 34.066 | 10.259 | 11.479 | 1.00 | 20.96 | N |
| ATOM | 741 | CA | LYS | A1098 | 33.228 | 9.456 | 10.578 | 1.00 | 20.77 | C |
| ATOM | 742 | CB | LYS | A1098 | 33.822 | 8.052 | 10.409 | 1.00 | 22.35 | C |
| ATOM | 743 | CG | LYS | A1098 | 35.205 | 8.017 | 9.774 | 1.00 | 25.18 | C |
| ATOM | 744 | CD | LYS | A1098 | 35.630 | 6.587 | 9.449 | 1.00 | 26.26 | C |
| ATOM | 745 | CE | LYS | A1098 | 36.996 | 6.553 | 8.772 | 1.00 | 28.39 | C |
| ATOM | 746 | NZ | LYS | A1098 | 37.332 | 5.194 | 8.258 | 1.00 | 28.94 | N |
| ATOM | 747 | C | LYS | A1098 | 31.770 | 9.313 | 11.046 | 1.00 | 19.74 | C |
| ATOM | 748 | O | LYS | A1098 | 31.517 | 9.064 | 12.224 | 1.00 | 19.90 | O |
| ATOM | 749 | N | ILE | A1099 | 30.826 | 9.461 | 10.113 | 1.00 | 18.27 | N |
| ATOM | 750 | CA | ILE | A1099 | 29.393 | 9.340 | 10.400 | 1.00 | 17.38 | C |
| ATOM | 751 | CB | ILE | A1099 | 28.622 | 10.638 | 9.997 | 1.00 | 17.39 | C |
| ATOM | 752 | CG2 | ILE | A1099 | 27.124 | 10.465 | 10.284 | 1.00 | 16.62 | C |
| ATOM | 753 | CG1 | ILE | A1099 | 29.171 | 11.849 | 10.761 | 1.00 | 15.42 | C |
| ATOM | 754 | CD1 | ILE | A1099 | 28.790 | 13.196 | 10.165 | 1.00 | 15.80 | C |
| ATOM | 755 | C | ILE | A1099 | 28.777 | 8.164 | 9.607 | 1.00 | 17.23 | C |
| ATOM | 756 | O | ILE | A1099 | 28.697 | 8.226 | 8.381 | 1.00 | 18.55 | O |
| ATOM | 757 | N | TRP | A1100 | 28.356 | 7.102 | 10.298 | 1.00 | 16.06 | N |
| ATOM | 758 | CA | TRP | A1100 | 27.733 | 5.939 | 9.641 | 1.00 | 15.73 | C |
| ATOM | 759 | CB | TRP | A1100 | 28.163 | 4.638 | 10.347 | 1.00 | 15.81 | C |
| ATOM | 760 | CG | TRP | A1100 | 29.665 | 4.374 | 10.328 | 1.00 | 16.55 | C |
| ATOM | 761 | CD2 | TRP | A1100 | 30.415 | 3.750 | 9.274 | 1.00 | 16.48 | C |
| ATOM | 762 | CE2 | TRP | A1100 | 31.774 | 3.736 | 9.676 | 1.00 | 17.61 | C |
| ATOM | 763 | CE3 | TRP | A1100 | 30.071 | 3.199 | 8.030 | 1.00 | 17.03 | C |
| ATOM | 764 | CD1 | TRP | A1100 | 30.575 | 4.704 | 11.300 | 1.00 | 17.00 | C |
| ATOM | 765 | NE1 | TRP | A1100 | 31.844 | 4.322 | 10.916 | 1.00 | 16.91 | N |
| ATOM | 766 | CZ2 | TRP | A1100 | 32.792 | 3.191 | 8.873 | 1.00 | 17.99 | C |
| ATOM | 767 | CZ3 | TRP | A1100 | 31.083 | 2.654 | 7.230 | 1.00 | 18.05 | C |
| ATOM | 768 | CH2 | TRP | A1100 | 32.430 | 2.656 | 7.659 | 1.00 | 17.40 | C |
| ATOM | 769 | C | TRP | A1100 | 26.190 | 6.079 | 9.705 | 1.00 | 15.39 | C |
| ATOM | 770 | O | TRP | A1100 | 25.638 | 6.160 | 10.801 | 1.00 | 15.11 | O |
| ATOM | 771 | N | LEU | A1101 | 25.517 | 6.100 | 8.545 | 1.00 | 14.54 | N |
| ATOM | 772 | CA | LEU | A1101 | 24.045 | 6.260 | 8.448 | 1.00 | 14.10 | C |
| ATOM | 773 | CB | LEU | A1101 | 23.705 | 7.658 | 7.883 | 1.00 | 14.83 | C |
| ATOM | 774 | CG | LEU | A1101 | 22.267 | 7.893 | 7.366 | 1.00 | 15.12 | C |
| ATOM | 775 | CD1 | LEU | A1101 | 21.330 | 8.116 | 8.541 | 1.00 | 15.77 | C |
| ATOM | 776 | CD2 | LEU | A1101 | 22.216 | 9.105 | 6.427 | 1.00 | 15.58 | C |
| ATOM | 777 | C | LEU | A1101 | 23.318 | 5.214 | 7.576 | 1.00 | 13.98 | C |
| ATOM | 778 | O | LEU | A1101 | 23.847 | 4.789 | 6.537 | 1.00 | 13.24 | O |
| ATOM | 779 | N | SER | A1102 | 22.109 | 4.808 | 7.996 | 1.00 | 13.49 | N |
| ATOM | 780 | CA | SER | A1102 | 21.278 | 3.857 | 7.226 | 1.00 | 14.16 | C |
| ATOM | 781 | CB | SER | A1102 | 21.525 | 2.411 | 7.683 | 1.00 | 14.32 | C |
| ATOM | 782 | OG | SER | A1102 | 20.947 | 1.471 | 6.781 | 1.00 | 14.30 | O |
| ATOM | 783 | C | SER | A1102 | 19.777 | 4.190 | 7.363 | 1.00 | 14.45 | C |
| ATOM | 784 | O | SER | A1102 | 19.311 | 4.519 | 8.464 | 1.00 | 13.54 | O |
| ATOM | 785 | N | ILE | A1103 | 19.029 | 4.089 | 6.254 | 1.00 | 14.69 | N |
| ATOM | 786 | CA | ILE | A1103 | 17.575 | 4.387 | 6.231 | 1.00 | 15.31 | C |
| ATOM | 787 | CB | ILE | A1103 | 17.282 | 5.709 | 5.454 | 1.00 | 15.58 | C |
| ATOM | 788 | CG2 | ILE | A1103 | 15.783 | 6.027 | 5.508 | 1.00 | 15.76 | C |
| ATOM | 789 | CG1 | ILE | A1103 | 18.085 | 6.875 | 6.041 | 1.00 | 15.76 | C |
| ATOM | 790 | CD1 | ILE | A1103 | 17.838 | 8.216 | 5.317 | 1.00 | 17.30 | C |
| ATOM | 791 | C | ILE | A1103 | 16.766 | 3.263 | 5.541 | 1.00 | 15.22 | C |
| ATOM | 792 | O | ILE | A1103 | 17.249 | 2.668 | 4.575 | 1.00 | 16.46 | O |
| ATOM | 793 | N | SER | A1104 | 15.550 | 2.977 | 6.023 | 1.00 | 15.94 | N |
| ATOM | 794 | CA | SER | A1104 | 14.686 | 1.926 | 5.429 | 1.00 | 17.08 | C |
| ATOM | 795 | CB | SER | A1104 | 14.993 | 0.564 | 6.085 | 1.00 | 17.67 | C |
| ATOM | 796 | OG | SER | A1104 | 14.251 | −0.486 | 5.476 | 1.00 | 17.18 | O |
| ATOM | 797 | C | SER | A1104 | 13.174 | 2.248 | 5.574 | 1.00 | 17.46 | C |
| ATOM | 798 | O | SER | A1104 | 12.784 | 2.982 | 6.483 | 1.00 | 16.57 | O |
| ATOM | 799 | N | HIS | A1105 | 12.323 | 1.699 | 4.698 | 1.00 | 18.34 | N |
| ATOM | 800 | CA | HIS | A1105 | 10.877 | 1.989 | 4.776 | 1.00 | 19.13 | C |
| ATOM | 801 | CB | HIS | A1105 | 10.572 | 3.300 | 4.041 | 1.00 | 20.61 | C |
| ATOM | 802 | CG | HIS | A1105 | 10.702 | 3.188 | 2.551 | 1.00 | 21.51 | C |
| ATOM | 803 | CD2 | HIS | A1105 | 9.799 | 2.840 | 1.601 | 1.00 | 22.68 | C |
| ATOM | 804 | ND1 | HIS | A1105 | 11.906 | 3.333 | 1.896 | 1.00 | 23.14 | N |
| ATOM | 805 | CE1 | HIS | A1105 | 11.742 | 3.075 | 0.610 | 1.00 | 23.36 | C |
| ATOM | 806 | NE2 | HIS | A1105 | 10.473 | 2.772 | 0.405 | 1.00 | 22.97 | N |
| ATOM | 807 | C | HIS | A1105 | 9.918 | 0.933 | 4.192 | 1.00 | 19.69 | C |
| ATOM | 808 | O | HIS | A1105 | 10.324 | 0.073 | 3.405 | 1.00 | 19.77 | O |
| ATOM | 809 | N | THR | A1106 | 8.640 | 1.034 | 4.577 | 1.00 | 20.07 | N |
| ATOM | 810 | CA | THR | A1106 | 7.564 | 0.171 | 4.056 | 1.00 | 20.65 | C |
| ATOM | 811 | CB | THR | A1106 | 6.941 | −0.769 | 5.131 | 1.00 | 20.02 | C |
| ATOM | 812 | OG1 | THR | A1106 | 6.198 | 0.004 | 6.085 | 1.00 | 19.30 | O |
| ATOM | 813 | CG2 | THR | A1106 | 8.029 | −1.578 | 5.852 | 1.00 | 19.90 | C |
| ATOM | 814 | C | THR | A1106 | 6.470 | 1.143 | 3.587 | 1.00 | 22.48 | C |
| ATOM | 815 | O | THR | A1106 | 6.720 | 2.347 | 3.476 | 1.00 | 21.87 | O |
| ATOM | 816 | N | ASP | A1107 | 5.262 | 0.652 | 3.315 | 1.00 | 23.59 | N |
| ATOM | 817 | CA | ASP | A1107 | 4.214 | 1.569 | 2.875 | 1.00 | 25.20 | C |
| ATOM | 818 | CB | ASP | A1107 | 3.146 | 0.831 | 2.052 | 1.00 | 27.87 | C |
| ATOM | 819 | CG | ASP | A1107 | 2.302 | −0.116 | 2.886 | 1.00 | 29.94 | C |
| ATOM | 820 | OD1 | ASP | A1107 | 1.331 | −0.672 | 2.332 | 1.00 | 32.47 | O |
| ATOM | 821 | OD2 | ASP | A1107 | 2.601 | −0.314 | 4.086 | 1.00 | 32.30 | O |
| ATOM | 822 | C | ASP | A1107 | 3.549 | 2.322 | 4.027 | 1.00 | 25.08 | C |
| ATOM | 823 | O | ASP | A1107 | 2.673 | 3.156 | 3.791 | 1.00 | 25.52 | O |
| ATOM | 824 | N | GLN | A1108 | 3.965 | 2.042 | 5.264 | 1.00 | 24.18 | N |
| ATOM | 825 | CA | GLN | A1108 | 3.380 | 2.703 | 6.435 | 1.00 | 23.78 | C |
| ATOM | 826 | CB | GLN | A1108 | 2.533 | 1.715 | 7.251 | 1.00 | 25.19 | C |
| ATOM | 827 | CG | GLN | A1108 | 1.283 | 1.211 | 6.550 | 1.00 | 28.60 | C |
| ATOM | 828 | CD | GLN | A1108 | 0.375 | 0.426 | 7.479 | 1.00 | 30.45 | C |
| ATOM | 829 | OE1 | GLN | A1108 | 0.768 | −0.606 | 8.026 | 1.00 | 31.65 | O |
| ATOM | 830 | NE2 | GLN | A1108 | −0.847 | 0.918 | 7.670 | 1.00 | 31.23 | N |
| ATOM | 831 | C | GLN | A1108 | 4.361 | 3.391 | 7.390 | 1.00 | 22.77 | C |
| ATOM | 832 | O | GLN | A1108 | 3.966 | 4.334 | 8.081 | 1.00 | 22.34 | O |
| ATOM | 833 | N | PHE | A1109 | 5.616 | 2.926 | 7.441 | 1.00 | 21.34 | N |
| ATOM | 834 | CA | PHE | A1109 | 6.633 | 3.513 | 8.342 | 1.00 | 20.67 | C |
| ATOM | 835 | CB | PHE | A1109 | 6.888 | 2.605 | 9.567 | 1.00 | 21.50 | C |
| ATOM | 836 | CG | PHE | A1109 | 5.654 | 2.213 | 10.328 | 1.00 | 22.53 | C |
| ATOM | 837 | CD1 | PHE | A1109 | 4.947 | 1.059 | 9.989 | 1.00 | 23.72 | C |
| ATOM | 838 | CD2 | PHE | A1109 | 5.212 | 2.978 | 11.403 | 1.00 | 23.05 | C |
| ATOM | 839 | CE1 | PHE | A1109 | 3.816 | 0.673 | 10.715 | 1.00 | 24.14 | C |
| ATOM | 840 | CE2 | PHE | A1109 | 4.082 | 2.601 | 12.135 | 1.00 | 24.05 | C |
| ATOM | 841 | CZ | PHE | A1109 | 3.383 | 1.445 | 11.789 | 1.00 | 23.64 | C |
| ATOM | 842 | C | PHE | A1109 | 8.015 | 3.755 | 7.705 | 1.00 | 19.29 | C |
| ATOM | 843 | O | PHE | A1109 | 8.333 | 3.199 | 6.654 | 1.00 | 19.32 | O |
| ATOM | 844 | N | VAL | A1110 | 8.830 | 4.582 | 8.368 | 1.00 | 18.27 | N |
| ATOM | 845 | CA | VAL | A1110 | 10.218 | 4.864 | 7.955 | 1.00 | 16.90 | C |
| ATOM | 846 | CB | VAL | A1110 | 10.368 | 6.279 | 7.299 | 1.00 | 17.79 | C |
| ATOM | 847 | CG1 | VAL | A1110 | 10.126 | 7.377 | 8.325 | 1.00 | 17.09 | C |
| ATOM | 848 | CG2 | VAL | A1110 | 11.754 | 6.428 | 6.681 | 1.00 | 17.98 | C |
| ATOM | 849 | C | VAL | A1110 | 11.100 | 4.796 | 9.224 | 1.00 | 16.47 | C |
| ATOM | 850 | O | VAL | A1110 | 10.655 | 5.214 | 10.300 | 1.00 | 15.21 | O |
| ATOM | 851 | N | THR | A1111 | 12.323 | 4.260 | 9.097 | 1.00 | 15.44 | N |
| ATOM | 852 | CA | THR | A1111 | 13.271 | 4.126 | 10.225 | 1.00 | 16.10 | C |
| ATOM | 853 | CB | THR | A1111 | 13.369 | 2.646 | 10.736 | 1.00 | 16.89 | C |
| ATOM | 854 | OG1 | THR | A1111 | 13.775 | 1.775 | 9.667 | 1.00 | 18.14 | O |
| ATOM | 855 | CG2 | THR | A1111 | 12.038 | 2.180 | 11.274 | 1.00 | 18.11 | C |
| ATOM | 856 | C | THR | A1111 | 14.692 | 4.582 | 9.860 | 1.00 | 15.27 | C |
| ATOM | 857 | O | THR | A1111 | 15.082 | 4.502 | 8.690 | 1.00 | 14.78 | O |
| ATOM | 858 | N | ALA | A1112 | 15.462 | 5.041 | 10.858 | 1.00 | 14.23 | N |
| ATOM | 859 | CA | ALA | A1112 | 16.845 | 5.505 | 10.639 | 1.00 | 13.59 | C |
| ATOM | 860 | CB | ALA | A1112 | 16.855 | 7.001 | 10.293 | 1.00 | 12.71 | C |
| ATOM | 861 | C | ALA | A1112 | 17.764 | 5.255 | 11.843 | 1.00 | 12.99 | C |
| ATOM | 862 | O | ALA | A1112 | 17.310 | 5.303 | 12.993 | 1.00 | 12.81 | O |
| ATOM | 863 | N | SER | A1113 | 19.055 | 5.032 | 11.570 | 1.00 | 12.47 | N |
| ATOM | 864 | CA | SER | A1113 | 20.064 | 4.761 | 12.610 | 1.00 | 13.04 | C |
| ATOM | 865 | CB | SER | A1113 | 20.256 | 3.238 | 12.736 | 1.00 | 13.53 | C |
| ATOM | 866 | OG | SER | A1113 | 21.244 | 2.893 | 13.698 | 1.00 | 13.09 | O |
| ATOM | 867 | C | SER | A1113 | 21.427 | 5.444 | 12.318 | 1.00 | 13.78 | C |
| ATOM | 868 | O | SER | A1113 | 21.901 | 5.419 | 11.180 | 1.00 | 13.46 | O |
| ATOM | 869 | N | VAL | A1114 | 22.048 | 6.039 | 13.348 | 1.00 | 13.44 | N |
| ATOM | 870 | CA | VAL | A1114 | 23.349 | 6.735 | 13.208 | 1.00 | 12.85 | C |
| ATOM | 871 | CB | VAL | A1114 | 23.151 | 8.288 | 13.286 | 1.00 | 12.88 | C |
| ATOM | 872 | CG1 | VAL | A1114 | 24.496 | 9.005 | 13.384 | 1.00 | 12.82 | C |
| ATOM | 873 | CG2 | VAL | A1114 | 22.384 | 8.794 | 12.056 | 1.00 | 13.36 | C |
| ATOM | 874 | C | VAL | A1114 | 24.393 | 6.328 | 14.278 | 1.00 | 12.70 | C |
| ATOM | 875 | O | VAL | A1114 | 24.045 | 6.140 | 15.444 | 1.00 | 12.48 | O |
| ATOM | 876 | N | ILE | A1115 | 25.662 | 6.183 | 13.873 | 1.00 | 13.61 | N |
| ATOM | 877 | CA | ILE | A1115 | 26.765 | 5.846 | 14.800 | 1.00 | 13.97 | C |
| ATOM | 878 | CB | ILE | A1115 | 27.291 | 4.399 | 14.621 | 1.00 | 14.70 | C |
| ATOM | 879 | CG2 | ILE | A1115 | 28.393 | 4.124 | 15.653 | 1.00 | 15.55 | C |
| ATOM | 880 | CG1 | ILE | A1115 | 26.163 | 3.377 | 14.800 | 1.00 | 14.56 | C |
| ATOM | 881 | CD1 | ILE | A1115 | 26.580 | 1.948 | 14.433 | 1.00 | 14.04 | C |
| ATOM | 882 | C | ILE | A1115 | 27.940 | 6.786 | 14.502 | 1.00 | 15.39 | C |
| ATOM | 883 | O | ILE | A1115 | 28.372 | 6.879 | 13.348 | 1.00 | 14.30 | O |
| ATOM | 884 | N | LEU | A1116 | 28.456 | 7.465 | 15.537 | 1.00 | 15.93 | N |
| ATOM | 885 | CA | LEU | A1116 | 29.574 | 8.412 | 15.397 | 1.00 | 16.40 | C |
| ATOM | 886 | CB | LEU | A1116 | 29.300 | 9.680 | 16.235 | 1.00 | 16.45 | C |
| ATOM | 887 | CG | LEU | A1116 | 27.972 | 10.439 | 16.051 | 1.00 | 15.86 | C |
| ATOM | 888 | CD1 | LEU | A1116 | 27.935 | 11.642 | 16.994 | 1.00 | 16.47 | C |
| ATOM | 889 | CD2 | LEU | A1116 | 27.816 | 10.888 | 14.601 | 1.00 | 16.74 | C |
| ATOM | 890 | C | LEU | A1116 | 30.906 | 7.778 | 15.839 | 1.00 | 17.18 | C |
| ATOM | 891 | O | LEU | A1116 | 30.973 | 7.125 | 16.880 | 1.00 | 15.82 | O |
| ATOM | 892 | N | GLU | A1117 | 31.970 | 8.002 | 15.064 | 1.00 | 18.58 | N |
| ATOM | 893 | CA | GLU | A1117 | 33.278 | 7.414 | 15.362 | 1.00 | 21.21 | C |
| ATOM | 894 | CB | GLU | A1117 | 33.404 | 6.068 | 14.615 | 1.00 | 21.29 | C |
| ATOM | 895 | CG | GLU | A1117 | 34.793 | 5.403 | 14.659 | 1.00 | 21.49 | C |
| ATOM | 896 | CD | GLU | A1117 | 34.930 | 4.238 | 13.675 | 1.00 | 21.22 | C |
| ATOM | 897 | OE1 | GLU | A1117 | 34.538 | 4.390 | 12.500 | 1.00 | 20.57 | O |
| ATOM | 898 | OE2 | GLU | A1117 | 35.446 | 3.166 | 14.064 | 1.00 | 22.68 | O |
| ATOM | 899 | C | GLU | A1117 | 34.487 | 8.290 | 15.001 | 1.00 | 23.59 | C |
| ATOM | 900 | O | GLU | A1117 | 34.440 | 9.108 | 14.083 | 1.00 | 22.02 | O |
| ATOM | 901 | N | GLU | A1118 | 35.576 | 8.115 | 15.740 | 1.00 | 27.34 | N |
| ATOM | 902 | CA | GLU | A1118 | 36.801 | 8.839 | 15.429 | 1.00 | 31.25 | C |
| ATOM | 903 | CB | GLU | A1118 | 36.859 | 10.184 | 16.157 | 1.00 | 33.00 | C |
| ATOM | 904 | CG | GLU | A1118 | 36.747 | 10.131 | 17.651 | 1.00 | 35.45 | C |
| ATOM | 905 | CD | GLU | A1118 | 37.042 | 11.480 | 18.269 | 1.00 | 37.44 | C |
| ATOM | 906 | OE1 | GLU | A1118 | 36.389 | 12.470 | 17.874 | 1.00 | 38.60 | O |
| ATOM | 907 | OE2 | GLU | A1118 | 37.932 | 11.551 | 19.145 | 1.00 | 39.01 | O |
| ATOM | 908 | C | GLU | A1118 | 38.005 | 7.964 | 15.764 | 1.00 | 33.04 | C |
| ATOM | 909 | O | GLU | A1118 | 38.074 | 7.358 | 16.835 | 1.00 | 33.37 | O |
| ATOM | 910 | N | ASN | A1119 | 38.928 | 7.875 | 14.809 | 1.00 | 34.80 | N |
| ATOM | 911 | CA | ASN | A1119 | 40.132 | 7.063 | 14.945 | 1.00 | 36.61 | C |
| ATOM | 912 | CB | ASN | A1119 | 40.203 | 6.015 | 13.823 | 1.00 | 37.32 | C |
| ATOM | 913 | CG | ASN | A1119 | 38.862 | 5.356 | 13.537 | 1.00 | 38.38 | C |
| ATOM | 914 | OD1 | ASN | A1119 | 38.258 | 4.731 | 14.409 | 1.00 | 39.30 | O |
| ATOM | 915 | ND2 | ASN | A1119 | 38.393 | 5.492 | 12.302 | 1.00 | 38.79 | N |
| ATOM | 916 | C | ASN | A1119 | 41.354 | 7.972 | 14.845 | 1.00 | 37.29 | C |
| ATOM | 917 | O | ASN | A1119 | 42.132 | 7.795 | 13.885 | 1.00 | 37.98 | O |
| ATOM | 918 | OXT | ASN | A1119 | 41.513 | 8.857 | 15.710 | 1.00 | 38.37 | O |
| TER | 919 | | ASN | A1119 | | | | | | |
| ATOM | 920 | CB | MET | B2003 | 37.510 | −3.957 | 21.175 | 1.00 | 28.67 | C |
| ATOM | 921 | CG | MET | B2003 | 37.230 | −5.443 | 21.092 | 1.00 | 31.61 | C |
| ATOM | 922 | SD | MET | B2003 | 38.381 | −6.420 | 22.089 | 1.00 | 36.00 | S |
| ATOM | 923 | CE | MET | B2003 | 37.478 | −6.538 | 23.632 | 1.00 | 34.64 | C |
| ATOM | 924 | C | MET | B2003 | 35.111 | −3.318 | 20.851 | 1.00 | 24.52 | C |
| ATOM | 925 | O | MET | B2003 | 34.821 | −3.223 | 22.044 | 1.00 | 23.78 | O |
| ATOM | 926 | N | MET | B2003 | 36.911 | −1.645 | 20.536 | 1.00 | 26.75 | N |
| ATOM | 927 | CA | MET | B2003 | 36.543 | −3.080 | 20.373 | 1.00 | 26.43 | C |
| ATOM | 928 | N | ILE | B2004 | 34.220 | −3.611 | 19.907 | 1.00 | 23.33 | N |
| ATOM | 929 | CA | ILE | B2004 | 32.814 | −3.877 | 20.214 | 1.00 | 21.77 | C |
| ATOM | 930 | CB | ILE | B2004 | 31.948 | −3.818 | 18.923 | 1.00 | 22.25 | C |
| ATOM | 931 | CG2 | ILE | B2004 | 30.528 | −4.317 | 19.214 | 1.00 | 21.40 | C |
| ATOM | 932 | CG1 | ILE | B2004 | 31.936 | −2.387 | 18.370 | 1.00 | 22.04 | C |
| ATOM | 933 | CD1 | ILE | B2004 | 31.248 | −2.242 | 17.013 | 1.00 | 23.31 | C |
| ATOM | 934 | C | ILE | B2004 | 32.688 | −5.268 | 20.837 | 1.00 | 21.10 | C |
| ATOM | 935 | O | ILE | B2004 | 33.423 | −6.190 | 20.463 | 1.00 | 21.53 | O |
| ATOM | 936 | N | VAL | B2005 | 31.774 | −5.423 | 21.793 | 1.00 | 19.59 | N |
| ATOM | 937 | CA | VAL | B2005 | 31.577 | −6.725 | 22.423 | 1.00 | 18.29 | C |
| ATOM | 938 | CB | VAL | B2005 | 32.240 | −6.783 | 23.834 | 1.00 | 18.82 | C |
| ATOM | 939 | CG1 | VAL | B2005 | 33.741 | −6.518 | 23.712 | 1.00 | 18.55 | C |
| ATOM | 940 | CG2 | VAL | B2005 | 31.596 | −5.776 | 24.778 | 1.00 | 19.42 | C |
| ATOM | 941 | C | VAL | B2005 | 30.108 | −7.189 | 22.520 | 1.00 | 17.17 | C |
| ATOM | 942 | O | VAL | B2005 | 29.827 | −8.180 | 23.193 | 1.00 | 16.88 | O |
| ATOM | 943 | N | GLY | B2006 | 29.190 | −6.493 | 21.838 | 1.00 | 15.24 | N |
| ATOM | 944 | CA | GLY | B2006 | 27.776 | −6.881 | 21.849 | 1.00 | 14.41 | C |
| ATOM | 945 | C | GLY | B2006 | 26.800 | −5.899 | 21.191 | 1.00 | 13.76 | C |
| ATOM | 946 | O | GLY | B2006 | 27.067 | −4.703 | 21.177 | 1.00 | 12.82 | O |
| ATOM | 947 | N | HIS | B2007 | 25.667 | −6.388 | 20.664 | 1.00 | 13.79 | N |
| ATOM | 948 | CA | HIS | B2007 | 24.656 | −5.521 | 20.015 | 1.00 | 13.97 | C |
| ATOM | 949 | CB | HIS | B2007 | 25.046 | −5.296 | 18.534 | 1.00 | 13.17 | C |
| ATOM | 950 | CG | HIS | B2007 | 24.110 | −4.400 | 17.767 | 1.00 | 13.29 | C |
| ATOM | 951 | CD2 | HIS | B2007 | 23.648 | −4.481 | 16.494 | 1.00 | 13.60 | C |
| ATOM | 952 | ND1 | HIS | B2007 | 23.602 | −3.224 | 18.281 | 1.00 | 14.70 | N |
| ATOM | 953 | CE1 | HIS | B2007 | 22.871 | −2.619 | 17.357 | 1.00 | 15.43 | C |
| ATOM | 954 | NE2 | HIS | B2007 | 22.884 | −3.360 | 16.262 | 1.00 | 13.92 | N |
| ATOM | 955 | C | HIS | B2007 | 23.227 | −6.108 | 20.104 | 1.00 | 13.36 | C |
| ATOM | 956 | O | HIS | B2007 | 23.024 | −7.293 | 19.817 | 1.00 | 13.30 | O |
| ATOM | 957 | N | GLY | B2008 | 22.249 | −5.286 | 20.504 | 1.00 | 13.56 | N |
| ATOM | 958 | CA | GLY | B2008 | 20.870 | −5.758 | 20.603 | 1.00 | 13.42 | C |
| ATOM | 959 | C | GLY | B2008 | 19.760 | −4.724 | 20.407 | 1.00 | 13.33 | C |
| ATOM | 960 | O | GLY | B2008 | 19.921 | −3.566 | 20.786 | 1.00 | 13.08 | O |
| ATOM | 961 | N | ILE | B2009 | 18.630 | −5.143 | 19.827 | 1.00 | 13.48 | N |
| ATOM | 962 | CA | ILE | B2009 | 17.476 | −4.253 | 19.601 | 1.00 | 13.81 | C |
| ATOM | 963 | CB | ILE | B2009 | 17.327 | −3.864 | 18.086 | 1.00 | 14.27 | C |
| ATOM | 964 | CG2 | ILE | B2009 | 18.651 | −3.276 | 17.549 | 1.00 | 14.33 | C |
| ATOM | 965 | CG1 | ILE | B2009 | 16.931 | −5.091 | 17.257 | 1.00 | 14.49 | C |
| ATOM | 966 | CD1 | ILE | B2009 | 16.666 | −4.783 | 15.777 | 1.00 | 15.47 | C |
| ATOM | 967 | C | ILE | B2009 | 16.155 | −4.916 | 20.066 | 1.00 | 14.94 | C |
| ATOM | 968 | O | ILE | B2009 | 16.090 | −6.145 | 20.190 | 1.00 | 14.51 | O |
| ATOM | 969 | N | ASP | B2010 | 15.115 | −4.111 | 20.318 | 1.00 | 15.66 | N |
| ATOM | 970 | CA | ASP | B2010 | 13.806 | −4.644 | 20.746 | 1.00 | 17.47 | C |
| ATOM | 971 | CB | ASP | B2010 | 13.820 | −4.974 | 22.250 | 1.00 | 18.72 | C |
| ATOM | 972 | CG | ASP | B2010 | 12.474 | −5.527 | 22.757 | 1.00 | 19.74 | C |
| ATOM | 973 | OD1 | ASP | B2010 | 11.670 | −4.747 | 23.311 | 1.00 | 21.08 | O |
| ATOM | 974 | OD2 | ASP | B2010 | 12.214 | −6.740 | 22.593 | 1.00 | 19.62 | O |
| ATOM | 975 | C | ASP | B2010 | 12.623 | −3.709 | 20.447 | 1.00 | 18.58 | C |
| ATOM | 976 | O | ASP | B2010 | 12.769 | −2.483 | 20.472 | 1.00 | 17.47 | O |
| ATOM | 977 | N | ILE | B2011 | 11.465 | −4.304 | 20.148 | 1.00 | 19.97 | N |
| ATOM | 978 | CA | ILE | B2011 | 10.224 | −3.558 | 19.874 | 1.00 | 22.59 | C |
| ATOM | 979 | CB | ILE | B2011 | 9.666 | −3.817 | 18.459 | 1.00 | 23.44 | C |
| ATOM | 980 | CG2 | ILE | B2011 | 8.532 | −2.831 | 18.167 | 1.00 | 24.17 | C |
| ATOM | 981 | CG1 | ILE | B2011 | 10.752 | −3.688 | 17.411 | 1.00 | 23.83 | C |
| ATOM | 982 | CD1 | ILE | B2011 | 10.368 | −4.350 | 16.110 | 1.00 | 24.58 | C |
| ATOM | 983 | C | ILE | B2011 | 9.134 | −4.066 | 20.833 | 1.00 | 23.70 | C |
| ATOM | 984 | O | ILE | B2011 | 9.039 | −5.271 | 21.078 | 1.00 | 23.87 | O |
| ATOM | 985 | N | GLU | B2012 | 8.305 | −3.166 | 21.359 | 1.00 | 24.36 | N |
| ATOM | 986 | CA | GLU | B2012 | 7.232 | −3.569 | 22.270 | 1.00 | 25.44 | C |
| ATOM | 987 | CB | GLU | B2012 | 7.626 | −3.297 | 23.723 | 1.00 | 26.83 | C |
| ATOM | 988 | CG | GLU | B2012 | 6.659 | −3.896 | 24.741 | 1.00 | 29.96 | C |
| ATOM | 989 | CD | GLU | B2012 | 6.806 | −5.408 | 24.892 | 1.00 | 32.18 | C |
| ATOM | 990 | OE1 | GLU | B2012 | 7.094 | −6.098 | 23.888 | 1.00 | 33.01 | O |
| ATOM | 991 | OE2 | GLU | B2012 | 6.617 | −5.908 | 26.024 | 1.00 | 33.41 | O |
| ATOM | 992 | C | GLU | B2012 | 5.915 | −2.848 | 21.952 | 1.00 | 25.77 | C |
| ATOM | 993 | O | GLU | B2012 | 5.911 | −1.677 | 21.585 | 1.00 | 24.25 | O |
| ATOM | 994 | N | GLU | B2013 | 4.800 | −3.563 | 22.089 | 1.00 | 26.07 | N |
| ATOM | 995 | CA | GLU | B2013 | 3.483 | −2.997 | 21.806 | 1.00 | 26.79 | C |
| ATOM | 996 | CB | GLU | B2013 | 2.546 | −4.097 | 21.294 | 1.00 | 27.06 | C |
| ATOM | 997 | CG | GLU | B2013 | 3.025 | −4.777 | 20.014 | 1.00 | 27.29 | C |
| ATOM | 998 | CD | GLU | B2013 | 2.147 | −5.943 | 19.607 | 0.05 | 27.19 | C |
| ATOM | 999 | OE1 | GLU | B2013 | 0.920 | −5.750 | 19.479 | 0.05 | 27.19 | O |
| ATOM | 1000 | OE2 | GLU | B2013 | 2.684 | −7.054 | 19.413 | 0.05 | 27.19 | O |
| ATOM | 1001 | C | GLU | B2013 | 2.895 | −2.358 | 23.061 | 1.00 | 27.09 | C |
| ATOM | 1002 | O | GLU | B2013 | 2.816 | −2.998 | 24.113 | 1.00 | 27.20 | O |
| ATOM | 1003 | N | LEU | B2014 | 2.489 | −1.096 | 22.952 | 1.00 | 27.74 | N |
| ATOM | 1004 | CA | LEU | B2014 | 1.915 | −0.383 | 24.088 | 1.00 | 28.99 | C |
| ATOM | 1005 | CB | LEU | B2014 | 1.448 | 1.015 | 23.666 | 1.00 | 29.85 | C |
| ATOM | 1006 | CG | LEU | B2014 | 2.499 | 2.064 | 23.293 | 1.00 | 30.49 | C |
| ATOM | 1007 | CD1 | LEU | B2014 | 1.802 | 3.353 | 22.863 | 1.00 | 31.36 | C |
| ATOM | 1008 | CD2 | LEU | B2014 | 3.408 | 2.332 | 24.477 | 1.00 | 30.66 | C |
| ATOM | 1009 | C | LEU | B2014 | 0.742 | −1.144 | 24.705 | 1.00 | 29.62 | C |
| ATOM | 1010 | O | LEU | B2014 | 0.614 | −1.209 | 25.924 | 1.00 | 28.92 | O |
| ATOM | 1011 | N | ALA | B2015 | −0.103 | −1.723 | 23.855 | 1.00 | 30.68 | N |
| ATOM | 1012 | CA | ALA | B2015 | −1.273 | −2.468 | 24.312 | 1.00 | 31.89 | C |
| ATOM | 1013 | CB | ALA | B2015 | −2.062 | −2.992 | 23.106 | 1.00 | 31.63 | C |
| ATOM | 1014 | C | ALA | B2015 | −0.942 | −3.624 | 25.256 | 1.00 | 32.68 | C |
| ATOM | 1015 | O | ALA | B2015 | −1.705 | −3.913 | 26.180 | 1.00 | 32.95 | O |
| ATOM | 1016 | N | SER | B2016 | 0.192 | −4.282 | 25.026 | 1.00 | 32.98 | N |
| ATOM | 1017 | CA | SER | B2016 | 0.599 | −5.409 | 25.861 | 1.00 | 33.69 | C |
| ATOM | 1018 | CB | SER | B2016 | 1.758 | −6.157 | 25.205 | 1.00 | 34.30 | C |
| ATOM | 1019 | OG | SER | B2016 | 1.360 | −6.697 | 23.955 | 1.00 | 36.37 | O |
| ATOM | 1020 | C | SER | B2016 | 0.993 | −4.981 | 27.271 | 1.00 | 33.62 | C |
| ATOM | 1021 | O | SER | B2016 | 0.707 | −5.680 | 28.242 | 1.00 | 33.30 | O |
| ATOM | 1022 | N | ILE | B2017 | 1.656 | −3.835 | 27.384 | 1.00 | 33.85 | N |
| ATOM | 1023 | CA | ILE | B2017 | 2.056 | −3.339 | 28.694 | 1.00 | 34.29 | C |
| ATOM | 1024 | CB | ILE | B2017 | 2.995 | −2.115 | 28.571 | 1.00 | 34.08 | C |
| ATOM | 1025 | CG2 | ILE | B2017 | 3.350 | −1.588 | 29.958 | 1.00 | 33.83 | C |
| ATOM | 1026 | CG1 | ILE | B2017 | 4.264 | −2.501 | 27.800 | 1.00 | 33.75 | C |
| ATOM | 1027 | CD1 | ILE | B2017 | 5.080 | −3.611 | 28.449 | 1.00 | 33.98 | C |
| ATOM | 1028 | C | ILE | B2017 | 0.802 | −2.932 | 29.474 | 1.00 | 35.14 | C |
| ATOM | 1029 | O | ILE | B2017 | 0.714 | −3.143 | 30.683 | 1.00 | 34.67 | O |
| ATOM | 1030 | N | GLU | B2018 | −0.172 | −2.362 | 28.770 | 1.00 | 36.36 | N |
| ATOM | 1031 | CA | GLU | B2018 | −1.414 | −1.925 | 29.402 | 1.00 | 37.92 | C |
| ATOM | 1032 | CB | GLU | B2018 | −2.292 | −1.189 | 28.386 | 1.00 | 38.65 | C |
| ATOM | 1033 | CG | GLU | B2018 | −1.548 | −0.122 | 27.599 | 1.00 | 40.03 | C |
| ATOM | 1034 | CD | GLU | B2018 | −2.462 | 0.705 | 26.716 | 1.00 | 41.09 | C |
| ATOM | 1035 | OE1 | GLU | B2018 | −3.463 | 0.148 | 26.216 | 1.00 | 41.60 | O |
| ATOM | 1036 | OE2 | GLU | B2018 | −2.172 | 1.907 | 26.510 | 1.00 | 41.15 | O |
| ATOM | 1037 | C | GLU | B2018 | −2.189 | −3.093 | 30.011 | 1.00 | 38.32 | C |
| ATOM | 1038 | O | GLU | B2018 | −2.697 | −2.993 | 31.129 | 1.00 | 39.05 | O |
| ATOM | 1039 | N | SER | B2019 | −2.281 | −4.198 | 29.278 | 1.00 | 38.71 | N |
| ATOM | 1040 | CA | SER | B2019 | −2.989 | −5.375 | 29.771 | 1.00 | 39.10 | C |
| ATOM | 1041 | CB | SER | B2019 | −2.996 | −6.476 | 28.709 | 1.00 | 39.02 | C |
| ATOM | 1042 | OG | SER | B2019 | −3.734 | −6.082 | 27.568 | 1.00 | 39.39 | O |
| ATOM | 1043 | C | SER | B2019 | −2.332 | −5.903 | 31.044 | 1.00 | 39.56 | C |
| ATOM | 1044 | O | SER | B2019 | −3.013 | −6.228 | 32.016 | 1.00 | 39.07 | O |
| ATOM | 1045 | N | ALA | B2020 | −1.004 | −5.983 | 31.031 | 1.00 | 39.92 | N |
| ATOM | 1046 | CA | ALA | B2020 | −0.253 | −6.471 | 32.182 | 1.00 | 40.75 | C |
| ATOM | 1047 | CB | ALA | B2020 | 1.240 | −6.474 | 31.866 | 1.00 | 39.96 | C |
| ATOM | 1048 | C | ALA | B2020 | −0.531 | −5.614 | 33.415 | 1.00 | 41.51 | C |
| ATOM | 1049 | O | ALA | B2020 | −0.584 | −6.122 | 34.535 | 1.00 | 41.34 | O |
| ATOM | 1050 | N | VAL | B2021 | −0.704 | −4.314 | 33.202 | 1.00 | 42.82 | N |
| ATOM | 1051 | CA | VAL | B2021 | −0.985 | −3.390 | 34.295 | 1.00 | 44.39 | C |
| ATOM | 1052 | CB | VAL | B2021 | −0.767 | −1.922 | 33.855 | 1.00 | 44.31 | C |
| ATOM | 1053 | CG1 | VAL | B2021 | −1.303 | −0.971 | 34.913 | 1.00 | 44.07 | C |
| ATOM | 1054 | CG2 | VAL | B2021 | 0.718 | −1.663 | 33.627 | 1.00 | 44.22 | C |
| ATOM | 1055 | C | VAL | B2021 | −2.424 | −3.555 | 34.787 | 1.00 | 45.72 | C |
| ATOM | 1056 | O | VAL | B2021 | −2.673 | −3.604 | 35.992 | 1.00 | 45.74 | O |
| ATOM | 1057 | N | THR | B2022 | −3.365 | −3.642 | 33.851 | 1.00 | 47.27 | N |
| ATOM | 1058 | CA | THR | B2022 | −4.776 | −3.801 | 34.191 | 1.00 | 48.73 | C |
| ATOM | 1059 | CB | THR | B2022 | −5.661 | −3.833 | 32.919 | 1.00 | 49.02 | C |
| ATOM | 1060 | OG1 | THR | B2022 | −5.482 | −2.619 | 32.177 | 1.00 | 49.29 | O |
| ATOM | 1061 | CG2 | THR | B2022 | −7.134 | −3.968 | 33.293 | 1.00 | 49.42 | C |
| ATOM | 1062 | C | THR | B2022 | −4.988 | −5.093 | 34.977 | 1.00 | 49.61 | C |
| ATOM | 1063 | O | THR | B2022 | −5.953 | −5.225 | 35.733 | 1.00 | 49.90 | O |
| ATOM | 1064 | N | ARG | B2023 | −4.077 | −6.043 | 34.794 | 1.00 | 50.61 | N |
| ATOM | 1065 | CA | ARG | B2023 | −4.152 | −7.325 | 35.484 | 1.00 | 51.35 | C |
| ATOM | 1066 | CB | ARG | B2023 | −3.028 | −8.245 | 35.009 | 1.00 | 52.09 | C |
| ATOM | 1067 | CG | ARG | B2023 | −3.416 | −9.710 | 34.907 | 1.00 | 53.02 | C |
| ATOM | 1068 | CD | ARG | B2023 | −3.676 | −10.103 | 33.461 | 1.00 | 54.10 | C |
| ATOM | 1069 | NE | ARG | B2023 | −2.492 | −9.895 | 32.628 | 1.00 | 54.73 | N |
| ATOM | 1070 | CZ | ARG | B2023 | −2.419 | −10.194 | 31.334 | 1.00 | 55.21 | C |
| ATOM | 1071 | NH1 | ARG | B2023 | −3.465 | −10.720 | 30.710 | 1.00 | 55.43 | N |
| ATOM | 1072 | NH2 | ARG | B2023 | −1.296 | −9.967 | 30.663 | 1.00 | 55.24 | N |
| ATOM | 1073 | C | ARG | B2023 | −4.025 | −7.093 | 36.989 | 1.00 | 51.64 | C |
| ATOM | 1074 | O | ARG | B2023 | −4.448 | −7.924 | 37.794 | 1.00 | 51.91 | O |
| ATOM | 1075 | N | HIS | B2024 | −3.428 | −5.960 | 37.354 | 1.00 | 51.80 | N |
| ATOM | 1076 | CA | HIS | B2024 | −3.242 | −5.575 | 38.753 | 1.00 | 51.59 | C |
| ATOM | 1077 | CB | HIS | B2024 | −4.586 | −5.645 | 39.491 | 1.00 | 52.56 | C |
| ATOM | 1078 | CG | HIS | B2024 | −4.528 | −5.165 | 40.908 | 1.00 | 53.50 | C |
| ATOM | 1079 | CD2 | HIS | B2024 | −4.815 | −5.791 | 42.074 | 1.00 | 53.85 | C |
| ATOM | 1080 | ND1 | HIS | B2024 | −4.142 | −3.886 | 41.245 | 1.00 | 53.73 | N |
| ATOM | 1081 | CE1 | HIS | B2024 | −4.195 | −3.743 | 42.557 | 1.00 | 54.11 | C |
| ATOM | 1082 | NE2 | HIS | B2024 | −4.601 | −4.885 | 43.084 | 1.00 | 54.34 | N |
| ATOM | 1083 | C | HIS | B2024 | −2.207 | −6.427 | 39.490 | 1.00 | 50.92 | C |
| ATOM | 1084 | O | HIS | B2024 | −1.802 | −6.100 | 40.607 | 1.00 | 51.09 | O |
| ATOM | 1085 | N | GLU | B2025 | −1.777 | −7.511 | 38.853 | 1.00 | 49.85 | N |
| ATOM | 1086 | CA | GLU | B2025 | −0.805 | −8.436 | 39.429 | 1.00 | 48.60 | C |
| ATOM | 1087 | CB | GLU | B2025 | −0.133 | −9.231 | 38.309 | 1.00 | 49.70 | C |
| ATOM | 1088 | CG | GLU | B2025 | −1.109 | −9.812 | 37.300 | 1.00 | 50.84 | C |
| ATOM | 1089 | CD | GLU | B2025 | −0.430 | −10.676 | 36.255 | 1.00 | 51.43 | C |
| ATOM | 1090 | OE1 | GLU | B2025 | 0.084 | −11.754 | 36.617 | 1.00 | 52.08 | O |
| ATOM | 1091 | OE2 | GLU | B2025 | −0.408 | −10.276 | 35.073 | 1.00 | 51.96 | O |
| ATOM | 1092 | C | GLU | B2025 | 0.274 | −7.788 | 40.301 | 1.00 | 47.18 | C |
| ATOM | 1093 | O | GLU | B2025 | 0.636 | −8.322 | 41.350 | 1.00 | 47.28 | O |
| ATOM | 1094 | N | GLY | B2026 | 0.779 | −6.638 | 39.864 | 1.00 | 45.23 | N |
| ATOM | 1095 | CA | GLY | B2026 | 1.832 | −5.953 | 40.598 | 1.00 | 42.17 | C |
| ATOM | 1096 | C | GLY | B2026 | 3.021 | −5.830 | 39.663 | 1.00 | 39.71 | C |
| ATOM | 1097 | O | GLY | B2026 | 4.185 | −5.914 | 40.066 | 1.00 | 39.77 | O |
| ATOM | 1098 | N | PHE | B2027 | 2.695 | −5.632 | 38.392 | 1.00 | 37.19 | N |
| ATOM | 1099 | CA | PHE | B2027 | 3.667 | −5.509 | 37.315 | 1.00 | 34.55 | C |
| ATOM | 1100 | CB | PHE | B2027 | 2.922 | −5.189 | 36.014 | 1.00 | 34.52 | C |
| ATOM | 1101 | CG | PHE | B2027 | 3.752 | −5.355 | 34.777 | 1.00 | 34.13 | C |
| ATOM | 1102 | CD1 | PHE | B2027 | 4.207 | −6.613 | 34.393 | 1.00 | 34.08 | C |
| ATOM | 1103 | CD2 | PHE | B2027 | 4.067 | −4.258 | 33.986 | 1.00 | 34.08 | C |
| ATOM | 1104 | CE1 | PHE | B2027 | 4.963 | −6.776 | 33.237 | 1.00 | 34.11 | C |
| ATOM | 1105 | CE2 | PHE | B2027 | 4.825 | −4.409 | 32.824 | 1.00 | 34.27 | C |
| ATOM | 1106 | CZ | PHE | B2027 | 5.274 | −5.670 | 32.449 | 1.00 | 34.06 | C |
| ATOM | 1107 | C | PHE | B2027 | 4.755 | −4.459 | 37.559 | 1.00 | 32.71 | C |
| ATOM | 1108 | O | PHE | B2027 | 5.933 | −4.794 | 37.676 | 1.00 | 31.86 | O |
| ATOM | 1109 | N | ALA | B2028 | 4.352 | −3.192 | 37.631 | 1.00 | 30.82 | N |
| ATOM | 1110 | CA | ALA | B2028 | 5.284 | −2.082 | 37.831 | 1.00 | 29.20 | C |
| ATOM | 1111 | CB | ALA | B2028 | 4.506 | −0.787 | 38.047 | 1.00 | 29.12 | C |
| ATOM | 1112 | C | ALA | B2028 | 6.290 | −2.269 | 38.966 | 1.00 | 28.66 | C |
| ATOM | 1113 | O | ALA | B2028 | 7.451 | −1.884 | 38.836 | 1.00 | 27.76 | O |
| ATOM | 1114 | N | LYS | B2029 | 5.846 | −2.853 | 40.075 | 1.00 | 27.74 | N |
| ATOM | 1115 | CA | LYS | B2029 | 6.718 | −3.065 | 41.226 | 1.00 | 27.62 | C |
| ATOM | 1116 | CB | LYS | B2029 | 5.899 | −3.552 | 42.423 | 1.00 | 27.96 | C |
| ATOM | 1117 | CG | LYS | B2029 | 5.853 | −2.569 | 43.584 | 1.00 | 28.74 | C |
| ATOM | 1118 | CD | LYS | B2029 | 7.247 | −2.299 | 44.131 | 0.05 | 28.50 | C |
| ATOM | 1119 | CE | LYS | B2029 | 7.204 | −1.384 | 45.343 | 0.05 | 28.61 | C |
| ATOM | 1120 | NZ | LYS | B2029 | 6.454 | −1.997 | 46.474 | 0.05 | 28.56 | N |
| ATOM | 1121 | C | LYS | B2029 | 7.864 | −4.042 | 40.969 | 1.00 | 27.09 | C |
| ATOM | 1122 | O | LYS | B2029 | 8.937 | −3.910 | 41.555 | 1.00 | 27.52 | O |
| ATOM | 1123 | N | ARG | B2030 | 7.642 | −5.022 | 40.099 | 1.00 | 25.99 | N |
| ATOM | 1124 | CA | ARG | B2030 | 8.682 | −6.002 | 39.803 | 1.00 | 24.71 | C |
| ATOM | 1125 | CB | ARG | B2030 | 8.048 | −7.358 | 39.465 | 1.00 | 25.36 | C |
| ATOM | 1126 | CG | ARG | B2030 | 7.303 | −7.990 | 40.631 | 0.05 | 25.04 | C |
| ATOM | 1127 | CD | ARG | B2030 | 6.804 | −9.384 | 40.291 | 0.05 | 25.09 | C |
| ATOM | 1128 | NE | ARG | B2030 | 6.143 | −10.015 | 41.430 | 0.05 | 25.02 | N |
| ATOM | 1129 | CZ | ARG | B2030 | 5.651 | −11.249 | 41.421 | 0.05 | 25.00 | C |
| ATOM | 1130 | NH1 | ARG | B2030 | 5.742 | −11.996 | 40.329 | 0.05 | 24.97 | N |
| ATOM | 1131 | NH2 | ARG | B2030 | 5.067 | −11.739 | 42.506 | 0.05 | 24.97 | N |
| ATOM | 1132 | C | ARG | B2030 | 9.616 | −5.566 | 38.674 | 1.00 | 23.43 | C |
| ATOM | 1133 | O | ARG | B2030 | 10.763 | −6.008 | 38.613 | 1.00 | 22.95 | O |
| ATOM | 1134 | N | VAL | B2031 | 9.131 | −4.688 | 37.797 | 1.00 | 22.08 | N |
| ATOM | 1135 | CA | VAL | B2031 | 9.924 | −4.202 | 36.665 | 1.00 | 20.91 | C |
| ATOM | 1136 | CB | VAL | B2031 | 8.998 | −3.878 | 35.447 | 1.00 | 21.66 | C |
| ATOM | 1137 | CG1 | VAL | B2031 | 9.804 | −3.269 | 34.307 | 1.00 | 21.12 | C |
| ATOM | 1138 | CG2 | VAL | B2031 | 8.311 | −5.152 | 34.961 | 1.00 | 21.59 | C |
| ATOM | 1139 | C | VAL | B2031 | 10.806 | −2.971 | 36.946 | 1.00 | 20.08 | C |
| ATOM | 1140 | O | VAL | B2031 | 11.891 | −2.840 | 36.362 | 1.00 | 18.87 | O |
| ATOM | 1141 | N | LEU | B2032 | 10.362 | −2.096 | 37.851 | 1.00 | 18.43 | N |
| ATOM | 1142 | CA | LEU | B2032 | 11.093 | −0.857 | 38.170 | 1.00 | 18.33 | C |
| ATOM | 1143 | CB | LEU | B2032 | 10.123 | 0.333 | 38.094 | 1.00 | 18.11 | C |
| ATOM | 1144 | CG | LEU | B2032 | 9.264 | 0.562 | 36.836 | 1.00 | 17.77 | C |
| ATOM | 1145 | CD1 | LEU | B2032 | 8.288 | 1.710 | 37.095 | 1.00 | 18.63 | C |
| ATOM | 1146 | CD2 | LEU | B2032 | 10.141 | 0.893 | 35.642 | 1.00 | 18.26 | C |
| ATOM | 1147 | C | LEU | B2032 | 11.809 | −0.804 | 39.537 | 1.00 | 18.11 | C |
| ATOM | 1148 | O | LEU | B2032 | 11.313 | −1.351 | 40.528 | 1.00 | 17.53 | O |
| ATOM | 1149 | N | THR | B2033 | 12.964 | −0.133 | 39.589 | 1.00 | 17.46 | N |
| ATOM | 1150 | CA | THR | B2033 | 13.707 | 0.022 | 40.848 | 1.00 | 17.07 | C |
| ATOM | 1151 | CB | THR | B2033 | 15.178 | 0.466 | 40.617 | 1.00 | 17.19 | C |
| ATOM | 1152 | OG1 | THR | B2033 | 15.199 | 1.760 | 39.996 | 1.00 | 17.05 | O |
| ATOM | 1153 | CG2 | THR | B2033 | 15.919 | −0.544 | 39.733 | 1.00 | 15.96 | C |
| ATOM | 1154 | C | THR | B2033 | 13.010 | 1.107 | 41.686 | 1.00 | 17.66 | C |
| ATOM | 1155 | O | THR | B2033 | 12.042 | 1.720 | 41.234 | 1.00 | 17.87 | O |
| ATOM | 1156 | N | ALA | B2034 | 13.501 | 1.355 | 42.896 | 1.00 | 17.54 | N |
| ATOM | 1157 | CA | ALA | B2034 | 12.886 | 2.367 | 43.757 | 1.00 | 18.74 | C |
| ATOM | 1158 | CB | ALA | B2034 | 13.565 | 2.369 | 45.123 | 1.00 | 18.99 | C |
| ATOM | 1159 | C | ALA | B2034 | 12.925 | 3.771 | 43.145 | 1.00 | 18.65 | C |
| ATOM | 1160 | O | ALA | B2034 | 11.930 | 4.503 | 43.188 | 1.00 | 18.77 | O |
| ATOM | 1161 | N | LEU | B2035 | 14.066 | 4.148 | 42.572 | 1.00 | 18.26 | N |
| ATOM | 1162 | CA | LEU | B2035 | 14.216 | 5.470 | 41.962 | 1.00 | 19.09 | C |
| ATOM | 1163 | CB | LEU | B2035 | 15.697 | 5.742 | 41.665 | 1.00 | 20.60 | C |
| ATOM | 1164 | CG | LEU | B2035 | 16.154 | 7.182 | 41.435 | 1.00 | 22.12 | C |
| ATOM | 1165 | CD1 | LEU | B2035 | 15.976 | 8.015 | 42.714 | 1.00 | 23.52 | C |
| ATOM | 1166 | CD2 | LEU | B2035 | 17.618 | 7.173 | 41.031 | 1.00 | 24.30 | C |
| ATOM | 1167 | C | LEU | B2035 | 13.383 | 5.610 | 40.680 | 1.00 | 18.93 | C |
| ATOM | 1168 | O | LEU | B2035 | 12.801 | 6.670 | 40.430 | 1.00 | 19.57 | O |
| ATOM | 1169 | N | GLU | B2036 | 13.333 | 4.559 | 39.858 | 1.00 | 18.62 | N |
| ATOM | 1170 | CA | GLU | B2036 | 12.533 | 4.604 | 38.626 | 1.00 | 18.35 | C |
| ATOM | 1171 | CB | GLU | B2036 | 12.733 | 3.327 | 37.784 | 1.00 | 17.51 | C |
| ATOM | 1172 | CG | GLU | B2036 | 14.085 | 3.215 | 37.042 | 1.00 | 15.99 | C |
| ATOM | 1173 | CD | GLU | B2036 | 14.300 | 1.848 | 36.391 | 1.00 | 16.12 | C |
| ATOM | 1174 | OE1 | GLU | B2036 | 15.007 | 1.777 | 35.361 | 1.00 | 15.21 | O |
| ATOM | 1175 | OE2 | GLU | B2036 | 13.781 | 0.836 | 36.911 | 1.00 | 15.16 | O |
| ATOM | 1176 | C | GLU | B2036 | 11.039 | 4.744 | 38.982 | 1.00 | 19.23 | C |
| ATOM | 1177 | O | GLU | B2036 | 10.290 | 5.440 | 38.296 | 1.00 | 19.60 | O |
| ATOM | 1178 | N | MET | B2037 | 10.616 | 4.074 | 40.051 | 1.00 | 19.86 | N |
| ATOM | 1179 | CA | MET | B2037 | 9.219 | 4.123 | 40.501 | 1.00 | 21.88 | C |
| ATOM | 1180 | CB | MET | B2037 | 9.008 | 3.086 | 41.615 | 1.00 | 22.85 | C |
| ATOM | 1181 | CG | MET | B2037 | 7.623 | 3.055 | 42.236 | 1.00 | 25.86 | C |
| ATOM | 1182 | SD | MET | B2037 | 6.356 | 2.391 | 41.143 | 1.00 | 28.53 | S |
| ATOM | 1183 | CE | MET | B2037 | 6.759 | 0.626 | 41.201 | 1.00 | 27.97 | C |
| ATOM | 1184 | C | MET | B2037 | 8.834 | 5.528 | 41.000 | 1.00 | 22.03 | C |
| ATOM | 1185 | O | MET | B2037 | 7.716 | 6.006 | 40.772 | 1.00 | 22.35 | O |
| ATOM | 1186 | N | GLU | B2038 | 9.759 | 6.187 | 41.687 | 1.00 | 22.54 | N |
| ATOM | 1187 | CA | GLU | B2038 | 9.501 | 7.528 | 42.194 | 1.00 | 23.34 | C |
| ATOM | 1188 | CB | GLU | B2038 | 10.663 | 7.973 | 43.101 | 1.00 | 23.49 | C |
| ATOM | 1189 | CG | GLU | B2038 | 10.475 | 7.496 | 44.550 | 1.00 | 24.52 | C |
| ATOM | 1190 | CD | GLU | B2038 | 11.772 | 7.284 | 45.326 | 1.00 | 25.50 | C |
| ATOM | 1191 | OE1 | GLU | B2038 | 12.785 | 7.962 | 45.036 | 1.00 | 25.67 | O |
| ATOM | 1192 | OE2 | GLU | B2038 | 11.765 | 6.439 | 46.251 | 1.00 | 25.94 | O |
| ATOM | 1193 | C | GLU | B2038 | 9.252 | 8.513 | 41.046 | 1.00 | 23.18 | C |
| ATOM | 1194 | O | GLU | B2038 | 8.512 | 9.482 | 41.200 | 1.00 | 23.42 | O |
| ATOM | 1195 | N | ARG | B2039 | 9.854 | 8.250 | 39.888 | 1.00 | 23.18 | N |
| ATOM | 1196 | CA | ARG | B2039 | 9.659 | 9.097 | 38.713 | 1.00 | 23.42 | C |
| ATOM | 1197 | CB | ARG | B2039 | 10.801 | 8.888 | 37.710 | 1.00 | 24.21 | C |
| ATOM | 1198 | CG | ARG | B2039 | 10.566 | 9.517 | 36.338 | 1.00 | 24.82 | C |
| ATOM | 1199 | CD | ARG | B2039 | 10.414 | 11.027 | 36.415 | 0.05 | 24.52 | C |
| ATOM | 1200 | NE | ARG | B2039 | 10.169 | 11.609 | 35.099 | 0.05 | 24.58 | N |
| ATOM | 1201 | CZ | ARG | B2039 | 9.995 | 12.908 | 34.873 | 0.05 | 24.54 | C |
| ATOM | 1202 | NH1 | ARG | B2039 | 9.776 | 13.344 | 33.640 | 0.05 | 24.55 | N |
| ATOM | 1203 | NH2 | ARG | B2039 | 10.039 | 13.771 | 35.879 | 0.05 | 24.55 | N |
| ATOM | 1204 | C | ARG | B2039 | 8.323 | 8.738 | 38.057 | 1.00 | 22.97 | C |
| ATOM | 1205 | O | ARG | B2039 | 7.591 | 9.614 | 37.594 | 1.00 | 23.62 | O |
| ATOM | 1206 | N | PHE | B2040 | 8.015 | 7.443 | 38.037 | 1.00 | 22.59 | N |
| ATOM | 1207 | CA | PHE | B2040 | 6.777 | 6.914 | 37.456 | 1.00 | 23.04 | C |
| ATOM | 1208 | CB | PHE | B2040 | 6.769 | 5.381 | 37.598 | 1.00 | 23.27 | C |
| ATOM | 1209 | CG | PHE | B2040 | 5.520 | 4.705 | 37.082 | 1.00 | 24.49 | C |
| ATOM | 1210 | CD1 | PHE | B2040 | 5.270 | 4.604 | 35.716 | 1.00 | 24.75 | C |
| ATOM | 1211 | CD2 | PHE | B2040 | 4.611 | 4.125 | 37.971 | 1.00 | 24.92 | C |
| ATOM | 1212 | CE1 | PHE | B2040 | 4.135 | 3.930 | 35.240 | 1.00 | 25.22 | C |
| ATOM | 1213 | CE2 | PHE | B2040 | 3.473 | 3.451 | 37.509 | 1.00 | 24.72 | C |
| ATOM | 1214 | CZ | PHE | B2040 | 3.236 | 3.353 | 36.139 | 1.00 | 25.48 | C |
| ATOM | 1215 | C | PHE | B2040 | 5.529 | 7.506 | 38.126 | 1.00 | 23.15 | C |
| ATOM | 1216 | O | PHE | B2040 | 4.558 | 7.841 | 37.448 | 1.00 | 22.73 | O |
| ATOM | 1217 | N | THR | B2041 | 5.559 | 7.636 | 39.452 | 1.00 | 23.63 | N |
| ATOM | 1218 | CA | THR | B2041 | 4.407 | 8.165 | 40.186 | 1.00 | 25.06 | C |
| ATOM | 1219 | CB | THR | B2041 | 4.487 | 7.819 | 41.701 | 1.00 | 24.62 | C |
| ATOM | 1220 | OG1 | THR | B2041 | 5.710 | 8.322 | 42.250 | 1.00 | 24.25 | O |
| ATOM | 1221 | CG2 | THR | B2041 | 4.421 | 6.311 | 41.910 | 1.00 | 24.17 | C |
| ATOM | 1222 | C | THR | B2041 | 4.156 | 9.669 | 40.050 | 1.00 | 25.84 | C |
| ATOM | 1223 | O | THR | B2041 | 3.089 | 10.155 | 40.442 | 1.00 | 26.22 | O |
| ATOM | 1224 | N | SER | B2042 | 5.119 | 10.412 | 39.508 | 1.00 | 27.24 | N |
| ATOM | 1225 | CA | SER | B2042 | 4.939 | 11.856 | 39.346 | 1.00 | 28.70 | C |
| ATOM | 1226 | CB | SER | B2042 | 6.256 | 12.605 | 39.605 | 1.00 | 29.90 | C |
| ATOM | 1227 | OG | SER | B2042 | 7.260 | 12.270 | 38.658 | 1.00 | 31.49 | O |
| ATOM | 1228 | C | SER | B2042 | 4.404 | 12.214 | 37.959 | 1.00 | 29.44 | C |
| ATOM | 1229 | O | SER | B2042 | 4.245 | 13.392 | 37.632 | 1.00 | 28.96 | O |
| ATOM | 1230 | N | LEU | B2043 | 4.127 | 11.192 | 37.151 | 1.00 | 29.93 | N |
| ATOM | 1231 | CA | LEU | B2043 | 3.598 | 11.379 | 35.797 | 1.00 | 30.98 | C |
| ATOM | 1232 | CB | LEU | B2043 | 4.466 | 10.622 | 34.779 | 1.00 | 30.83 | C |
| ATOM | 1233 | CG | LEU | B2043 | 5.925 | 11.061 | 34.614 | 1.00 | 30.66 | C |
| ATOM | 1234 | CD1 | LEU | B2043 | 6.690 | 10.037 | 33.778 | 1.00 | 31.31 | C |
| ATOM | 1235 | CD2 | LEU | B2043 | 5.970 | 12.431 | 33.960 | 1.00 | 31.38 | C |
| ATOM | 1236 | C | LEU | B2043 | 2.160 | 10.864 | 35.712 | 1.00 | 31.75 | C |
| ATOM | 1237 | O | LEU | B2043 | 1.711 | 10.106 | 36.573 | 1.00 | 31.45 | O |
| ATOM | 1238 | N | LYS | B2044 | 1.440 | 11.264 | 34.668 | 1.00 | 33.21 | N |
| ATOM | 1239 | CA | LYS | B2044 | 0.059 | 10.820 | 34.497 | 1.00 | 34.53 | C |
| ATOM | 1240 | CB | LYS | B2044 | −0.904 | 11.853 | 35.100 | 1.00 | 35.00 | C |
| ATOM | 1241 | CG | LYS | B2044 | −2.362 | 11.415 | 35.118 | 0.05 | 34.86 | C |
| ATOM | 1242 | CD | LYS | B2044 | −3.267 | 12.481 | 35.714 | 0.05 | 34.96 | C |
| ATOM | 1243 | CE | LYS | B2044 | −4.718 | 12.025 | 35.718 | 0.05 | 34.95 | C |
| ATOM | 1244 | NZ | LYS | B2044 | −5.625 | 13.056 | 36.293 | 0.05 | 35.07 | N |
| ATOM | 1245 | C | LYS | B2044 | −0.311 | 10.580 | 33.035 | 1.00 | 35.45 | C |
| ATOM | 1246 | O | LYS | B2044 | 0.422 | 10.960 | 32.121 | 1.00 | 35.43 | O |
| ATOM | 1247 | N | GLY | B2045 | −1.454 | 9.934 | 32.829 | 1.00 | 36.33 | N |
| ATOM | 1248 | CA | GLY | B2045 | −1.940 | 9.671 | 31.488 | 1.00 | 37.45 | C |
| ATOM | 1249 | C | GLY | B2045 | −1.031 | 8.869 | 30.579 | 1.00 | 38.02 | C |
| ATOM | 1250 | O | GLY | B2045 | −0.215 | 8.065 | 31.036 | 1.00 | 38.35 | O |
| ATOM | 1251 | N | ARG | B2046 | −1.187 | 9.099 | 29.279 | 1.00 | 38.11 | N |
| ATOM | 1252 | CA | ARG | B2046 | −0.414 | 8.409 | 28.250 | 1.00 | 38.00 | C |
| ATOM | 1253 | CB | ARG | B2046 | −0.718 | 9.026 | 26.882 | 1.00 | 38.22 | C |
| ATOM | 1254 | CG | ARG | B2046 | −2.203 | 9.241 | 26.635 | 0.05 | 38.32 | C |
| ATOM | 1255 | CD | ARG | B2046 | −2.469 | 9.951 | 25.319 | 0.05 | 38.47 | C |
| ATOM | 1256 | NE | ARG | B2046 | −2.193 | 9.103 | 24.164 | 0.05 | 38.58 | N |
| ATOM | 1257 | CZ | ARG | B2046 | −2.418 | 9.462 | 22.904 | 0.05 | 38.64 | C |
| ATOM | 1258 | NH1 | ARG | B2046 | −2.138 | 8.626 | 21.915 | 0.05 | 38.67 | N |
| ATOM | 1259 | NH2 | ARG | B2046 | −2.925 | 10.657 | 22.633 | 0.05 | 38.67 | N |
| ATOM | 1260 | C | ARG | B2046 | 1.083 | 8.472 | 28.516 | 1.00 | 37.79 | C |
| ATOM | 1261 | O | ARG | B2046 | 1.809 | 7.514 | 28.262 | 1.00 | 37.64 | O |
| ATOM | 1262 | N | ARG | B2047 | 1.534 | 9.611 | 29.031 | 1.00 | 37.35 | N |
| ATOM | 1263 | CA | ARG | B2047 | 2.945 | 9.827 | 29.327 | 1.00 | 36.86 | C |
| ATOM | 1264 | CB | ARG | B2047 | 3.139 | 11.262 | 29.824 | 1.00 | 38.35 | C |
| ATOM | 1265 | CG | ARG | B2047 | 4.529 | 11.834 | 29.622 | 1.00 | 40.01 | C |
| ATOM | 1266 | CD | ARG | B2047 | 4.591 | 13.258 | 30.156 | 1.00 | 41.52 | C |
| ATOM | 1267 | NE | ARG | B2047 | 5.880 | 13.894 | 29.903 | 1.00 | 43.12 | N |
| ATOM | 1268 | CZ | ARG | B2047 | 6.276 | 15.030 | 30.471 | 1.00 | 44.06 | C |
| ATOM | 1269 | NH1 | ARG | B2047 | 7.467 | 15.539 | 30.180 | 1.00 | 44.67 | N |
| ATOM | 1270 | NH2 | ARG | B2047 | 5.486 | 15.654 | 31.336 | 1.00 | 44.50 | N |
| ATOM | 1271 | C | ARG | B2047 | 3.460 | 8.826 | 30.368 | 1.00 | 35.56 | C |
| ATOM | 1272 | O | ARG | B2047 | 4.631 | 8.454 | 30.354 | 1.00 | 34.93 | O |
| ATOM | 1273 | N | GLN | B2048 | 2.578 | 8.388 | 31.263 | 1.00 | 34.21 | N |
| ATOM | 1274 | CA | GLN | B2048 | 2.947 | 7.433 | 32.305 | 1.00 | 32.54 | C |
| ATOM | 1275 | CB | GLN | B2048 | 1.866 | 7.413 | 33.396 | 1.00 | 33.03 | C |
| ATOM | 1276 | CG | GLN | B2048 | 2.203 | 6.564 | 34.616 | 1.00 | 32.53 | C |
| ATOM | 1277 | CD | GLN | B2048 | 1.203 | 6.741 | 35.751 | 1.00 | 33.31 | C |
| ATOM | 1278 | OE1 | GLN | B2048 | 0.009 | 6.499 | 35.586 | 1.00 | 32.97 | O |
| ATOM | 1279 | NE2 | GLN | B2048 | 1.694 | 7.164 | 36.914 | 1.00 | 33.17 | N |
| ATOM | 1280 | C | GLN | B2048 | 3.161 | 6.021 | 31.740 | 1.00 | 31.47 | C |
| ATOM | 1281 | O | GLN | B2048 | 4.119 | 5.340 | 32.106 | 1.00 | 31.22 | O |
| ATOM | 1282 | N | ILE | B2049 | 2.269 | 5.589 | 30.852 | 1.00 | 29.87 | N |
| ATOM | 1283 | CA | ILE | B2049 | 2.366 | 4.266 | 30.233 | 1.00 | 28.47 | C |
| ATOM | 1284 | CB | ILE | B2049 | 1.087 | 3.936 | 29.417 | 1.00 | 28.92 | C |
| ATOM | 1285 | CG2 | ILE | B2049 | 1.263 | 2.627 | 28.667 | 1.00 | 29.21 | C |
| ATOM | 1286 | CG1 | ILE | B2049 | −0.130 | 3.858 | 30.346 | 1.00 | 29.32 | C |
| ATOM | 1287 | CD1 | ILE | B2049 | −0.118 | 2.675 | 31.305 | 1.00 | 28.99 | C |
| ATOM | 1288 | C | ILE | B2049 | 3.577 | 4.191 | 29.292 | 1.00 | 27.84 | C |
| ATOM | 1289 | O | ILE | B2049 | 4.172 | 3.127 | 29.116 | 1.00 | 27.30 | O |
| ATOM | 1290 | N | GLU | B2050 | 3.933 | 5.321 | 28.688 | 1.00 | 26.72 | N |
| ATOM | 1291 | CA | GLU | B2050 | 5.073 | 5.373 | 27.774 | 1.00 | 26.84 | C |
| ATOM | 1292 | CB | GLU | B2050 | 5.060 | 6.692 | 26.985 | 1.00 | 27.77 | C |
| ATOM | 1293 | CG | GLU | B2050 | 6.298 | 6.973 | 26.119 | 1.00 | 30.05 | C |
| ATOM | 1294 | CD | GLU | B2050 | 6.456 | 6.027 | 24.927 | 1.00 | 31.34 | C |
| ATOM | 1295 | OE1 | GLU | B2050 | 5.443 | 5.718 | 24.262 | 1.00 | 32.49 | O |
| ATOM | 1296 | OE2 | GLU | B2050 | 7.603 | 5.610 | 24.643 | 1.00 | 31.18 | O |
| ATOM | 1297 | C | GLU | B2050 | 6.397 | 5.223 | 28.520 | 1.00 | 25.29 | C |
| ATOM | 1298 | O | GLU | B2050 | 7.339 | 4.621 | 27.994 | 1.00 | 24.70 | O |
| ATOM | 1299 | N | TYR | B2051 | 6.471 | 5.767 | 29.737 | 1.00 | 23.32 | N |
| ATOM | 1300 | CA | TYR | B2051 | 7.696 | 5.675 | 30.537 | 1.00 | 21.39 | C |
| ATOM | 1301 | CB | TYR | B2051 | 7.576 | 6.526 | 31.821 | 1.00 | 21.45 | C |
| ATOM | 1302 | CG | TYR | B2051 | 8.819 | 6.522 | 32.712 | 1.00 | 20.60 | C |
| ATOM | 1303 | CD1 | TYR | B2051 | 8.974 | 5.577 | 33.728 | 1.00 | 20.47 | C |
| ATOM | 1304 | CE1 | TYR | B2051 | 10.128 | 5.547 | 34.526 | 1.00 | 20.26 | C |
| ATOM | 1305 | CD2 | TYR | B2051 | 9.852 | 7.444 | 32.512 | 1.00 | 20.44 | C |
| ATOM | 1306 | CE2 | TYR | B2051 | 11.012 | 7.420 | 33.302 | 1.00 | 20.69 | C |
| ATOM | 1307 | CZ | TYR | B2051 | 11.141 | 6.469 | 34.306 | 1.00 | 20.69 | C |
| ATOM | 1308 | OH | TYR | B2051 | 12.277 | 6.449 | 35.094 | 1.00 | 21.32 | O |
| ATOM | 1309 | C | TYR | B2051 | 7.978 | 4.212 | 30.885 | 1.00 | 20.75 | C |
| ATOM | 1310 | O | TYR | B2051 | 9.085 | 3.718 | 30.658 | 1.00 | 19.49 | O |
| ATOM | 1311 | N | LEU | B2052 | 6.968 | 3.520 | 31.413 | 1.00 | 19.18 | N |
| ATOM | 1312 | CA | LEU | B2052 | 7.096 | 2.109 | 31.788 | 1.00 | 18.14 | C |
| ATOM | 1313 | CB | LEU | B2052 | 5.782 | 1.616 | 32.419 | 1.00 | 18.69 | C |
| ATOM | 1314 | CG | LEU | B2052 | 5.668 | 0.154 | 32.868 | 1.00 | 19.58 | C |
| ATOM | 1315 | CD1 | LEU | B2052 | 6.783 | −0.189 | 33.861 | 1.00 | 19.24 | C |
| ATOM | 1316 | CD2 | LEU | B2052 | 4.299 | −0.070 | 33.511 | 1.00 | 20.41 | C |
| ATOM | 1317 | C | LEU | B2052 | 7.476 | 1.209 | 30.598 | 1.00 | 17.54 | C |
| ATOM | 1318 | O | LEU | B2052 | 8.347 | 0.347 | 30.719 | 1.00 | 16.53 | O |
| ATOM | 1319 | N | ALA | B2053 | 6.828 | 1.411 | 29.453 | 1.00 | 16.29 | N |
| ATOM | 1320 | CA | ALA | B2053 | 7.115 | 0.600 | 28.262 | 1.00 | 16.39 | C |
| ATOM | 1321 | CB | ALA | B2053 | 6.106 | 0.926 | 27.154 | 1.00 | 16.13 | C |
| ATOM | 1322 | C | ALA | B2053 | 8.550 | 0.794 | 27.744 | 1.00 | 15.79 | C |
| ATOM | 1323 | O | ALA | B2053 | 9.206 | −0.169 | 27.325 | 1.00 | 16.28 | O |
| ATOM | 1324 | N | GLY | B2054 | 9.034 | 2.033 | 27.771 | 1.00 | 15.48 | N |
| ATOM | 1325 | CA | GLY | B2054 | 10.391 | 2.311 | 27.316 | 1.00 | 15.32 | C |
| ATOM | 1326 | C | GLY | B2054 | 11.471 | 1.701 | 28.201 | 1.00 | 16.07 | C |
| ATOM | 1327 | O | GLY | B2054 | 12.550 | 1.309 | 27.716 | 1.00 | 14.96 | O |
| ATOM | 1328 | N | ARG | B2055 | 11.208 | 1.633 | 29.506 | 1.00 | 15.22 | N |
| ATOM | 1329 | CA | ARG | B2055 | 12.179 | 1.040 | 30.430 | 1.00 | 16.35 | C |
| ATOM | 1330 | CB | ARG | B2055 | 11.790 | 1.353 | 31.886 | 1.00 | 17.42 | C |
| ATOM | 1331 | CG | ARG | B2055 | 12.545 | 2.543 | 32.514 | 1.00 | 19.58 | C |
| ATOM | 1332 | CD | ARG | B2055 | 12.545 | 3.794 | 31.623 | 1.00 | 21.46 | C |
| ATOM | 1333 | NE | ARG | B2055 | 13.423 | 4.838 | 32.153 | 1.00 | 21.36 | N |
| ATOM | 1334 | CZ | ARG | B2055 | 13.695 | 5.986 | 31.534 | 1.00 | 22.03 | C |
| ATOM | 1335 | NH1 | ARG | B2055 | 13.155 | 6.256 | 30.353 | 1.00 | 22.33 | N |
| ATOM | 1336 | NH2 | ARG | B2055 | 14.520 | 6.865 | 32.094 | 1.00 | 21.30 | N |
| ATOM | 1337 | C | ARG | B2055 | 12.261 | −0.479 | 30.194 | 1.00 | 16.15 | C |
| ATOM | 1338 | O | ARG | B2055 | 13.339 | −1.079 | 30.277 | 1.00 | 15.13 | O |
| ATOM | 1339 | N | TRP | B2056 | 11.119 | −1.090 | 29.884 | 1.00 | 16.30 | N |
| ATOM | 1340 | CA | TRP | B2056 | 11.055 | −2.528 | 29.608 | 1.00 | 17.26 | C |
| ATOM | 1341 | CB | TRP | B2056 | 9.592 | −2.966 | 29.425 | 1.00 | 19.31 | C |
| ATOM | 1342 | CG | TRP | B2056 | 9.393 | −4.449 | 29.444 | 1.00 | 22.46 | C |
| ATOM | 1343 | CD2 | TRP | B2056 | 9.458 | −5.335 | 28.325 | 1.00 | 24.24 | C |
| ATOM | 1344 | CE2 | TRP | B2056 | 9.234 | −6.644 | 28.813 | 1.00 | 25.47 | C |
| ATOM | 1345 | CE3 | TRP | B2056 | 9.684 | −5.153 | 26.954 | 1.00 | 25.79 | C |
| ATOM | 1346 | CD1 | TRP | B2056 | 9.139 | −5.234 | 30.540 | 1.00 | 23.87 | C |
| ATOM | 1347 | NE1 | TRP | B2056 | 9.041 | −6.552 | 30.167 | 1.00 | 24.64 | N |
| ATOM | 1348 | CZ2 | TRP | B2056 | 9.231 | −7.763 | 27.979 | 1.00 | 26.38 | C |
| ATOM | 1349 | CZ3 | TRP | B2056 | 9.681 | −6.270 | 26.122 | 1.00 | 27.06 | C |
| ATOM | 1350 | CH2 | TRP | B2056 | 9.455 | −7.559 | 26.641 | 1.00 | 27.05 | C |
| ATOM | 1351 | C | TRP | B2056 | 11.843 | −2.850 | 28.327 | 1.00 | 16.16 | C |
| ATOM | 1352 | O | TRP | B2056 | 12.642 | −3.794 | 28.288 | 1.00 | 16.26 | O |
| ATOM | 1353 | N | SER | B2057 | 11.608 | −2.053 | 27.288 | 1.00 | 15.01 | N |
| ATOM | 1354 | CA | SER | B2057 | 12.265 | −2.208 | 25.980 | 1.00 | 14.82 | C |
| ATOM | 1355 | CB | SER | B2057 | 11.678 | −1.155 | 25.017 | 1.00 | 15.72 | C |
| ATOM | 1356 | OG | SER | B2057 | 12.069 | −1.352 | 23.670 | 1.00 | 19.47 | O |
| ATOM | 1357 | C | SER | B2057 | 13.807 | −2.052 | 26.087 | 1.00 | 14.21 | C |
| ATOM | 1358 | O | SER | B2057 | 14.581 | −2.816 | 25.471 | 1.00 | 12.39 | O |
| ATOM | 1359 | N | ALA | B2058 | 14.246 | −1.072 | 26.873 | 1.00 | 12.21 | N |
| ATOM | 1360 | CA | ALA | B2058 | 15.680 | −0.810 | 27.052 | 1.00 | 12.92 | C |
| ATOM | 1361 | CB | ALA | B2058 | 15.878 | 0.516 | 27.800 | 1.00 | 12.17 | C |
| ATOM | 1362 | C | ALA | B2058 | 16.398 | −1.949 | 27.801 | 1.00 | 12.59 | C |
| ATOM | 1363 | O | ALA | B2058 | 17.507 | −2.354 | 27.428 | 1.00 | 11.75 | O |
| ATOM | 1364 | N | LYS | B2059 | 15.772 | −2.455 | 28.861 | 1.00 | 12.47 | N |
| ATOM | 1365 | CA | LYS | B2059 | 16.368 | −3.546 | 29.634 | 1.00 | 13.35 | C |
| ATOM | 1366 | CB | LYS | B2059 | 15.579 | −3.757 | 30.947 | 1.00 | 14.19 | C |
| ATOM | 1367 | CG | LYS | B2059 | 15.656 | −2.538 | 31.896 | 1.00 | 14.71 | C |
| ATOM | 1368 | CD | LYS | B2059 | 14.961 | −2.754 | 33.258 | 1.00 | 15.04 | C |
| ATOM | 1369 | CE | LYS | B2059 | 15.045 | −1.488 | 34.124 | 1.00 | 16.38 | C |
| ATOM | 1370 | NZ | LYS | B2059 | 14.590 | −1.651 | 35.545 | 1.00 | 15.99 | N |
| ATOM | 1371 | C | LYS | B2059 | 16.459 | −4.846 | 28.814 | 1.00 | 14.13 | C |
| ATOM | 1372 | O | LYS | B2059 | 17.406 | −5.632 | 28.992 | 1.00 | 13.94 | O |
| ATOM | 1373 | N | GLU | B2060 | 15.500 | −5.072 | 27.907 | 1.00 | 13.55 | N |
| ATOM | 1374 | CA | GLU | B2060 | 15.535 | −6.276 | 27.066 | 1.00 | 14.73 | C |
| ATOM | 1375 | CB | GLU | B2060 | 14.160 | −6.552 | 26.436 | 1.00 | 16.24 | C |
| ATOM | 1376 | CG | GLU | B2060 | 14.173 | −7.696 | 25.413 | 1.00 | 20.48 | C |
| ATOM | 1377 | CD | GLU | B2060 | 14.283 | −9.089 | 26.037 | 1.00 | 23.43 | C |
| ATOM | 1378 | OE1 | GLU | B2060 | 14.802 | −9.230 | 27.168 | 1.00 | 26.30 | O |
| ATOM | 1379 | OE2 | GLU | B2060 | 13.860 | −10.061 | 25.376 | 1.00 | 25.45 | O |
| ATOM | 1380 | C | GLU | B2060 | 16.608 | −6.174 | 25.960 | 1.00 | 13.95 | C |
| ATOM | 1381 | O | GLU | B2060 | 17.238 | −7.178 | 25.609 | 1.00 | 13.52 | O |
| ATOM | 1382 | N | ALA | B2061 | 16.819 | −4.971 | 25.418 | 1.00 | 13.22 | N |
| ATOM | 1383 | CA | ALA | B2061 | 17.838 | −4.765 | 24.379 | 1.00 | 13.62 | C |
| ATOM | 1384 | CB | ALA | B2061 | 17.740 | −3.332 | 23.801 | 1.00 | 13.16 | C |
| ATOM | 1385 | C | ALA | B2061 | 19.241 | −5.022 | 24.950 | 1.00 | 13.67 | C |
| ATOM | 1386 | O | ALA | B2061 | 20.107 | −5.576 | 24.257 | 1.00 | 14.02 | O |
| ATOM | 1387 | N | PHE | B2062 | 19.463 | −4.630 | 26.207 | 1.00 | 13.22 | N |
| ATOM | 1388 | CA | PHE | B2062 | 20.760 | −4.860 | 26.873 | 1.00 | 14.50 | C |
| ATOM | 1389 | CB | PHE | B2062 | 20.844 | −4.049 | 28.185 | 1.00 | 14.44 | C |
| ATOM | 1390 | CG | PHE | B2062 | 22.074 | −4.353 | 29.029 | 1.00 | 14.90 | C |
| ATOM | 1391 | CD1 | PHE | B2062 | 22.084 | −5.445 | 29.908 | 1.00 | 15.30 | C |
| ATOM | 1392 | CD2 | PHE | B2062 | 23.223 | −3.571 | 28.923 | 1.00 | 15.56 | C |
| ATOM | 1393 | CE1 | PHE | B2062 | 23.223 | −5.752 | 30.663 | 1.00 | 15.94 | C |
| ATOM | 1394 | CE2 | PHE | B2062 | 24.375 | −3.869 | 29.678 | 1.00 | 15.34 | C |
| ATOM | 1395 | CZ | PHE | B2062 | 24.370 | −4.961 | 30.545 | 1.00 | 15.50 | C |
| ATOM | 1396 | C | PHE | B2062 | 20.969 | −6.363 | 27.147 | 1.00 | 14.72 | C |
| ATOM | 1397 | O | PHE | B2062 | 22.063 | −6.894 | 26.938 | 1.00 | 14.93 | O |
| ATOM | 1398 | N | SER | B2063 | 19.922 | −7.052 | 27.599 | 1.00 | 14.63 | N |
| ATOM | 1399 | CA | SER | B2063 | 20.016 | −8.490 | 27.864 | 1.00 | 15.27 | C |
| ATOM | 1400 | CB | SER | B2063 | 18.688 | −9.021 | 28.415 | 1.00 | 16.20 | C |
| ATOM | 1401 | OG | SER | B2063 | 18.406 | −8.473 | 29.694 | 1.00 | 18.16 | O |
| ATOM | 1402 | C | SER | B2063 | 20.379 | −9.257 | 26.579 | 1.00 | 15.62 | C |
| ATOM | 1403 | O | SER | B2063 | 21.187 | −10.185 | 26.607 | 1.00 | 14.45 | O |
| ATOM | 1404 | N | LYS | B2064 | 19.767 | −8.872 | 25.459 | 1.00 | 15.61 | N |
| ATOM | 1405 | CA | LYS | B2064 | 20.049 | −9.514 | 24.175 | 1.00 | 16.52 | C |
| ATOM | 1406 | CB | LYS | B2064 | 19.012 | −9.077 | 23.125 | 1.00 | 16.00 | C |
| ATOM | 1407 | CG | LYS | B2064 | 17.614 | −9.664 | 23.369 | 1.00 | 15.31 | C |
| ATOM | 1408 | CD | LYS | B2064 | 16.563 | −9.149 | 22.365 | 1.00 | 17.09 | C |
| ATOM | 1409 | CE | LYS | B2064 | 16.849 | −9.635 | 20.935 | 1.00 | 18.35 | C |
| ATOM | 1410 | NZ | LYS | B2064 | 15.798 | −9.213 | 19.951 | 1.00 | 19.10 | N |
| ATOM | 1411 | C | LYS | B2064 | 21.470 | −9.195 | 23.694 | 1.00 | 17.20 | C |
| ATOM | 1412 | O | LYS | B2064 | 22.113 | −10.024 | 23.043 | 1.00 | 17.94 | O |
| ATOM | 1413 | N | ALA | B2065 | 21.962 | −7.999 | 24.014 | 1.00 | 18.06 | N |
| ATOM | 1414 | CA | ALA | B2065 | 23.316 | −7.605 | 23.623 | 1.00 | 19.56 | C |
| ATOM | 1415 | CB | ALA | B2065 | 23.545 | −6.119 | 23.922 | 1.00 | 20.35 | C |
| ATOM | 1416 | C | ALA | B2065 | 24.361 | −8.453 | 24.358 | 1.00 | 21.22 | C |
| ATOM | 1417 | O | ALA | B2065 | 25.473 | −8.657 | 23.859 | 1.00 | 20.91 | O |
| ATOM | 1418 | N | MET | B2066 | 24.007 | −8.940 | 25.545 | 1.00 | 22.79 | N |
| ATOM | 1419 | CA | MET | B2066 | 24.924 | −9.774 | 26.331 | 1.00 | 25.40 | C |
| ATOM | 1420 | CB | MET | B2066 | 24.690 | −9.573 | 27.836 | 1.00 | 25.98 | C |
| ATOM | 1421 | CG | MET | B2066 | 25.035 | −8.178 | 28.353 | 1.00 | 28.04 | C |
| ATOM | 1422 | SD | MET | B2066 | 26.596 | −8.065 | 29.288 | 1.00 | 31.65 | S |
| ATOM | 1423 | CE | MET | B2066 | 26.043 | −8.670 | 30.858 | 1.00 | 29.68 | C |
| ATOM | 1424 | C | MET | B2066 | 24.761 | −11.256 | 25.983 | 1.00 | 26.28 | C |
| ATOM | 1425 | O | MET | B2066 | 25.539 | −12.095 | 26.441 | 1.00 | 27.12 | O |
| ATOM | 1426 | N | GLY | B2067 | 23.743 | −11.570 | 25.184 | 1.00 | 26.98 | N |
| ATOM | 1427 | CA | GLY | B2067 | 23.501 | −12.943 | 24.772 | 1.00 | 28.85 | C |
| ATOM | 1428 | C | GLY | B2067 | 22.447 | −13.728 | 25.544 | 1.00 | 30.30 | C |
| ATOM | 1429 | O | GLY | B2067 | 22.379 | −14.950 | 25.408 | 1.00 | 30.22 | O |
| ATOM | 1430 | N | THR | B2068 | 21.618 | −13.048 | 26.335 | 1.00 | 31.36 | N |
| ATOM | 1431 | CA | THR | B2068 | 20.583 | −13.729 | 27.120 | 1.00 | 33.00 | C |
| ATOM | 1432 | CB | THR | B2068 | 21.043 | −13.892 | 28.595 | 1.00 | 32.89 | C |
| ATOM | 1433 | OG1 | THR | B2068 | 20.057 | −14.628 | 29.332 | 1.00 | 33.56 | O |
| ATOM | 1434 | CG2 | THR | B2068 | 21.253 | −12.528 | 29.248 | 1.00 | 32.58 | C |
| ATOM | 1435 | C | THR | B2068 | 19.217 | −13.023 | 27.083 | 1.00 | 33.93 | C |
| ATOM | 1436 | O | THR | B2068 | 18.867 | −12.381 | 26.091 | 1.00 | 34.69 | O |
| ATOM | 1437 | N | GLY | B2069 | 18.446 | −13.157 | 28.161 | 1.00 | 35.37 | N |
| ATOM | 1438 | CA | GLY | B2069 | 17.132 | −12.534 | 28.239 | 1.00 | 36.59 | C |
| ATOM | 1439 | C | GLY | B2069 | 16.890 | −11.870 | 29.586 | 1.00 | 37.70 | C |
| ATOM | 1440 | O | GLY | B2069 | 17.525 | −12.221 | 30.580 | 1.00 | 38.22 | O |
| ATOM | 1441 | N | ILE | B2070 | 15.960 | −10.918 | 29.631 | 1.00 | 38.74 | N |
| ATOM | 1442 | CA | ILE | B2070 | 15.668 | −10.195 | 30.869 | 1.00 | 39.48 | C |
| ATOM | 1443 | CB | ILE | B2070 | 14.789 | −8.948 | 30.595 | 1.00 | 39.69 | C |
| ATOM | 1444 | CG2 | ILE | B2070 | 13.430 | −9.375 | 30.057 | 1.00 | 40.00 | C |
| ATOM | 1445 | CG1 | ILE | B2070 | 14.621 | −8.132 | 31.880 | 1.00 | 39.65 | C |
| ATOM | 1446 | CD1 | ILE | B2070 | 15.923 | −7.652 | 32.491 | 1.00 | 39.07 | C |
| ATOM | 1447 | C | ILE | B2070 | 15.008 | −11.043 | 31.960 | 1.00 | 40.17 | C |
| ATOM | 1448 | O | ILE | B2070 | 15.155 | −10.748 | 33.150 | 1.00 | 40.22 | O |
| ATOM | 1449 | N | SER | B2071 | 14.283 | −12.087 | 31.564 | 1.00 | 40.66 | N |
| ATOM | 1450 | CA | SER | B2071 | 13.632 | −12.963 | 32.539 | 1.00 | 41.23 | C |
| ATOM | 1451 | CB | SER | B2071 | 12.702 | −13.965 | 31.841 | 1.00 | 41.77 | C |
| ATOM | 1452 | OG | SER | B2071 | 11.623 | −13.319 | 31.185 | 1.00 | 43.00 | O |
| ATOM | 1453 | C | SER | B2071 | 14.694 | −13.729 | 33.324 | 1.00 | 41.05 | C |
| ATOM | 1454 | O | SER | B2071 | 14.446 | −14.202 | 34.435 | 1.00 | 41.50 | O |
| ATOM | 1455 | N | LYS | B2072 | 15.883 | −13.838 | 32.740 | 1.00 | 40.66 | N |
| ATOM | 1456 | CA | LYS | B2072 | 16.992 | −14.554 | 33.360 | 1.00 | 39.81 | C |
| ATOM | 1457 | CB | LYS | B2072 | 17.754 | −15.336 | 32.286 | 1.00 | 40.65 | C |
| ATOM | 1458 | CG | LYS | B2072 | 16.868 | −16.186 | 31.383 | 1.00 | 41.38 | C |
| ATOM | 1459 | CD | LYS | B2072 | 17.686 | −16.871 | 30.301 | 1.00 | 42.22 | C |
| ATOM | 1460 | CE | LYS | B2072 | 16.818 | −17.749 | 29.419 | 1.00 | 42.85 | C |
| ATOM | 1461 | NZ | LYS | B2072 | 15.748 | −16.964 | 28.747 | 1.00 | 43.40 | N |
| ATOM | 1462 | C | LYS | B2072 | 17.954 | −13.620 | 34.093 | 1.00 | 38.99 | C |
| ATOM | 1463 | O | LYS | B2072 | 18.445 | −13.947 | 35.174 | 1.00 | 38.73 | O |
| ATOM | 1464 | N | LEU | B2073 | 18.214 | −12.457 | 33.502 | 1.00 | 38.00 | N |
| ATOM | 1465 | CA | LEU | B2073 | 19.136 | −11.480 | 34.082 | 1.00 | 37.21 | C |
| ATOM | 1466 | CB | LEU | B2073 | 19.514 | −10.444 | 33.015 | 1.00 | 37.84 | C |
| ATOM | 1467 | CG | LEU | B2073 | 20.842 | −9.689 | 33.144 | 1.00 | 38.45 | C |
| ATOM | 1468 | CD1 | LEU | B2073 | 21.202 | −9.069 | 31.801 | 1.00 | 38.77 | C |
| ATOM | 1469 | CD2 | LEU | B2073 | 20.738 | −8.625 | 34.226 | 1.00 | 38.87 | C |
| ATOM | 1470 | C | LEU | B2073 | 18.583 | −10.784 | 35.338 | 1.00 | 36.22 | C |
| ATOM | 1471 | O | LEU | B2073 | 19.335 | −10.491 | 36.268 | 1.00 | 36.38 | O |
| ATOM | 1472 | N | GLY | B2074 | 17.276 | −10.527 | 35.366 | 1.00 | 34.86 | N |
| ATOM | 1473 | CA | GLY | B2074 | 16.670 | −9.874 | 36.520 | 1.00 | 32.86 | C |
| ATOM | 1474 | C | GLY | B2074 | 16.457 | −8.376 | 36.351 | 1.00 | 31.51 | C |
| ATOM | 1475 | O | GLY | B2074 | 17.415 | −7.625 | 36.168 | 1.00 | 31.09 | O |
| ATOM | 1476 | N | PHE | B2075 | 15.204 | −7.934 | 36.435 | 1.00 | 29.86 | N |
| ATOM | 1477 | CA | PHE | B2075 | 14.873 | −6.516 | 36.274 | 1.00 | 28.90 | C |
| ATOM | 1478 | CB | PHE | B2075 | 13.348 | −6.309 | 36.264 | 1.00 | 29.85 | C |
| ATOM | 1479 | CG | PHE | B2075 | 12.656 | −6.885 | 35.062 | 1.00 | 30.57 | C |
| ATOM | 1480 | CD1 | PHE | B2075 | 12.145 | −8.177 | 35.090 | 1.00 | 31.57 | C |
| ATOM | 1481 | CD2 | PHE | B2075 | 12.502 | −6.130 | 33.905 | 1.00 | 31.69 | C |
| ATOM | 1482 | CE1 | PHE | B2075 | 11.487 | −8.714 | 33.980 | 1.00 | 31.82 | C |
| ATOM | 1483 | CE2 | PHE | B2075 | 11.845 | −6.656 | 32.786 | 1.00 | 32.40 | C |
| ATOM | 1484 | CZ | PHE | B2075 | 11.336 | −7.954 | 32.828 | 1.00 | 32.18 | C |
| ATOM | 1485 | C | PHE | B2075 | 15.470 | −5.558 | 37.310 | 1.00 | 27.58 | C |
| ATOM | 1486 | O | PHE | B2075 | 15.773 | −4.409 | 36.983 | 1.00 | 26.85 | O |
| ATOM | 1487 | N | GLN | B2076 | 15.626 | −6.013 | 38.551 | 1.00 | 26.42 | N |
| ATOM | 1488 | CA | GLN | B2076 | 16.161 | −5.156 | 39.612 | 1.00 | 26.02 | C |
| ATOM | 1489 | CB | GLN | B2076 | 15.829 | −5.745 | 40.995 | 1.00 | 26.15 | C |
| ATOM | 1490 | CG | GLN | B2076 | 14.332 | −5.838 | 41.292 | 1.00 | 25.92 | C |
| ATOM | 1491 | CD | GLN | B2076 | 13.641 | −4.482 | 41.284 | 1.00 | 26.13 | C |
| ATOM | 1492 | OE1 | GLN | B2076 | 12.615 | −4.300 | 40.624 | 1.00 | 25.80 | O |
| ATOM | 1493 | NE2 | GLN | B2076 | 14.193 | −3.528 | 42.031 | 1.00 | 25.89 | N |
| ATOM | 1494 | C | GLN | B2076 | 17.663 | −4.892 | 39.524 | 1.00 | 25.52 | C |
| ATOM | 1495 | O | GLN | B2076 | 18.195 | −4.068 | 40.276 | 1.00 | 25.38 | O |
| ATOM | 1496 | N | ASP | B2077 | 18.343 | −5.576 | 38.608 | 1.00 | 25.07 | N |
| ATOM | 1497 | CA | ASP | B2077 | 19.789 | −5.407 | 38.448 | 1.00 | 25.24 | C |
| ATOM | 1498 | CB | ASP | B2077 | 20.447 | −6.739 | 38.060 | 1.00 | 28.16 | C |
| ATOM | 1499 | CG | ASP | B2077 | 20.008 | −7.895 | 38.945 | 1.00 | 31.24 | C |
| ATOM | 1500 | OD1 | ASP | B2077 | 19.868 | −7.699 | 40.172 | 1.00 | 32.43 | O |
| ATOM | 1501 | OD2 | ASP | B2077 | 19.815 | −9.009 | 38.407 | 1.00 | 34.01 | O |
| ATOM | 1502 | C | ASP | B2077 | 20.164 | −4.350 | 37.402 | 1.00 | 23.27 | C |
| ATOM | 1503 | O | ASP | B2077 | 21.347 | −4.096 | 37.173 | 1.00 | 23.32 | O |
| ATOM | 1504 | N | LEU | B2078 | 19.160 | −3.742 | 36.774 | 1.00 | 21.03 | N |
| ATOM | 1505 | CA | LEU | B2078 | 19.376 | −2.717 | 35.746 | 1.00 | 18.79 | C |
| ATOM | 1506 | CB | LEU | B2078 | 18.923 | −3.240 | 34.370 | 1.00 | 18.82 | C |
| ATOM | 1507 | CG | LEU | B2078 | 19.560 | −4.543 | 33.861 | 1.00 | 19.30 | C |
| ATOM | 1508 | CD1 | LEU | B2078 | 18.877 | −5.001 | 32.575 | 1.00 | 19.23 | C |
| ATOM | 1509 | CD2 | LEU | B2078 | 21.040 | −4.325 | 33.622 | 1.00 | 19.43 | C |
| ATOM | 1510 | C | LEU | B2078 | 18.582 | −1.451 | 36.081 | 1.00 | 18.03 | C |
| ATOM | 1511 | O | LEU | B2078 | 17.463 | −1.543 | 36.591 | 1.00 | 17.56 | O |
| ATOM | 1512 | N | GLU | B2079 | 19.150 | −0.276 | 35.792 | 1.00 | 16.76 | N |
| ATOM | 1513 | CA | GLU | B2079 | 18.466 | 0.998 | 36.060 | 1.00 | 15.78 | C |
| ATOM | 1514 | CB | GLU | B2079 | 18.912 | 1.563 | 37.417 | 1.00 | 16.39 | C |
| ATOM | 1515 | CG | GLU | B2079 | 18.136 | 2.809 | 37.863 | 1.00 | 18.61 | C |
| ATOM | 1516 | CD | GLU | B2079 | 18.567 | 3.319 | 39.237 | 1.00 | 19.54 | C |
| ATOM | 1517 | OE1 | GLU | B2079 | 19.725 | 3.776 | 39.376 | 1.00 | 18.25 | O |
| ATOM | 1518 | OE2 | GLU | B2079 | 17.743 | 3.257 | 40.180 | 1.00 | 20.67 | O |
| ATOM | 1519 | C | GLU | B2079 | 18.729 | 2.029 | 34.944 | 1.00 | 15.93 | C |
| ATOM | 1520 | O | GLU | B2079 | 19.884 | 2.233 | 34.543 | 1.00 | 15.32 | O |
| ATOM | 1521 | N | VAL | B2080 | 17.659 | 2.666 | 34.452 | 1.00 | 15.15 | N |
| ATOM | 1522 | CA | VAL | B2080 | 17.735 | 3.664 | 33.371 | 1.00 | 15.13 | C |
| ATOM | 1523 | CB | VAL | B2080 | 16.973 | 3.169 | 32.083 | 1.00 | 14.90 | C |
| ATOM | 1524 | CG1 | VAL | B2080 | 17.063 | 4.228 | 30.963 | 1.00 | 12.93 | C |
| ATOM | 1525 | CG2 | VAL | B2080 | 17.555 | 1.844 | 31.593 | 1.00 | 14.67 | C |
| ATOM | 1526 | C | VAL | B2080 | 17.120 | 5.015 | 33.779 | 1.00 | 16.52 | C |
| ATOM | 1527 | O | VAL | B2080 | 15.913 | 5.096 | 34.046 | 1.00 | 16.43 | O |
| ATOM | 1528 | N | LEU | B2081 | 17.941 | 6.069 | 33.815 | 1.00 | 17.23 | N |
| ATOM | 1529 | CA | LEU | B2081 | 17.469 | 7.414 | 34.178 | 1.00 | 18.35 | C |
| ATOM | 1530 | CB | LEU | B2081 | 18.248 | 7.944 | 35.398 | 1.00 | 18.31 | C |
| ATOM | 1531 | CG | LEU | B2081 | 18.457 | 7.034 | 36.621 | 1.00 | 18.74 | C |
| ATOM | 1532 | CD1 | LEU | B2081 | 19.403 | 7.722 | 37.602 | 1.00 | 19.49 | C |
| ATOM | 1533 | CD2 | LEU | B2081 | 17.128 | 6.720 | 37.296 | 1.00 | 19.00 | C |
| ATOM | 1534 | C | LEU | B2081 | 17.657 | 8.391 | 33.006 | 1.00 | 19.38 | C |
| ATOM | 1535 | O | LEU | B2081 | 18.257 | 8.040 | 31.984 | 1.00 | 18.07 | O |
| ATOM | 1536 | N | ASN | B2082 | 17.148 | 9.616 | 33.159 | 1.00 | 21.21 | N |
| ATOM | 1537 | CA | ASN | B2082 | 17.276 | 10.659 | 32.125 | 1.00 | 23.15 | C |
| ATOM | 1538 | CB | ASN | B2082 | 15.898 | 11.236 | 31.770 | 1.00 | 24.29 | C |
| ATOM | 1539 | CG | ASN | B2082 | 15.110 | 10.351 | 30.816 | 1.00 | 25.10 | C |
| ATOM | 1540 | OD1 | ASN | B2082 | 15.401 | 10.292 | 29.619 | 1.00 | 26.00 | O |
| ATOM | 1541 | ND2 | ASN | B2082 | 14.109 | 9.657 | 31.343 | 1.00 | 24.28 | N |
| ATOM | 1542 | C | ASN | B2082 | 18.171 | 11.785 | 32.654 | 1.00 | 23.63 | C |
| ATOM | 1543 | O | ASN | B2082 | 17.988 | 12.228 | 33.785 | 1.00 | 24.09 | O |
| ATOM | 1544 | N | ASN | B2083 | 19.137 | 12.260 | 31.864 | 1.00 | 24.20 | N |
| ATOM | 1545 | CA | ASN | B2083 | 20.001 | 13.328 | 32.376 | 1.00 | 25.17 | C |
| ATOM | 1546 | CB | ASN | B2083 | 21.395 | 13.280 | 31.711 | 1.00 | 25.74 | C |
| ATOM | 1547 | CG | ASN | B2083 | 21.405 | 13.770 | 30.270 | 1.00 | 25.64 | C |
| ATOM | 1548 | OD1 | ASN | B2083 | 22.425 | 13.652 | 29.584 | 1.00 | 27.52 | O |
| ATOM | 1549 | ND2 | ASN | B2083 | 20.299 | 14.327 | 29.810 | 1.00 | 24.85 | N |
| ATOM | 1550 | C | ASN | B2083 | 19.356 | 14.718 | 32.279 | 1.00 | 25.54 | C |
| ATOM | 1551 | O | ASN | B2083 | 18.214 | 14.844 | 31.831 | 1.00 | 25.60 | O |
| ATOM | 1552 | N | GLU | B2084 | 20.067 | 15.759 | 32.706 | 1.00 | 26.28 | N |
| ATOM | 1553 | CA | GLU | B2084 | 19.490 | 17.105 | 32.688 | 1.00 | 27.23 | C |
| ATOM | 1554 | CB | GLU | B2084 | 20.378 | 18.083 | 33.479 | 1.00 | 28.30 | C |
| ATOM | 1555 | CG | GLU | B2084 | 21.731 | 18.411 | 32.879 | 1.00 | 29.51 | C |
| ATOM | 1556 | CD | GLU | B2084 | 22.612 | 19.175 | 33.860 | 1.00 | 31.00 | C |
| ATOM | 1557 | OE1 | GLU | B2084 | 22.065 | 19.983 | 34.645 | 1.00 | 31.91 | O |
| ATOM | 1558 | OE2 | GLU | B2084 | 23.847 | 18.976 | 33.847 | 1.00 | 30.61 | O |
| ATOM | 1559 | C | GLU | B2084 | 19.153 | 17.675 | 31.307 | 1.00 | 27.45 | C |
| ATOM | 1560 | O | GLU | B2084 | 18.514 | 18.725 | 31.207 | 1.00 | 27.41 | O |
| ATOM | 1561 | N | ARG | B2085 | 19.569 | 16.986 | 30.244 | 1.00 | 27.72 | N |
| ATOM | 1562 | CA | ARG | B2085 | 19.259 | 17.429 | 28.885 | 1.00 | 27.29 | C |
| ATOM | 1563 | CB | ARG | B2085 | 20.490 | 17.355 | 27.976 | 1.00 | 29.07 | C |
| ATOM | 1564 | CG | ARG | B2085 | 21.545 | 18.411 | 28.244 | 1.00 | 30.86 | C |
| ATOM | 1565 | CD | ARG | B2085 | 22.540 | 18.483 | 27.089 | 1.00 | 33.01 | C |
| ATOM | 1566 | NE | ARG | B2085 | 23.592 | 19.471 | 27.319 | 1.00 | 34.52 | N |
| ATOM | 1567 | CZ | ARG | B2085 | 24.556 | 19.337 | 28.221 | 1.00 | 34.97 | C |
| ATOM | 1568 | NH1 | ARG | B2085 | 24.608 | 18.252 | 28.982 | 1.00 | 36.01 | N |
| ATOM | 1569 | NH2 | ARG | B2085 | 25.469 | 20.285 | 28.363 | 1.00 | 35.52 | N |
| ATOM | 1570 | C | ARG | B2085 | 18.147 | 16.563 | 28.294 | 1.00 | 26.76 | C |
| ATOM | 1571 | O | ARG | B2085 | 17.642 | 16.838 | 27.203 | 1.00 | 26.29 | O |
| ATOM | 1572 | N | GLY | B2086 | 17.777 | 15.508 | 29.018 | 1.00 | 25.36 | N |
| ATOM | 1573 | CA | GLY | B2086 | 16.721 | 14.623 | 28.558 | 1.00 | 24.19 | C |
| ATOM | 1574 | C | GLY | B2086 | 17.156 | 13.325 | 27.896 | 1.00 | 23.02 | C |
| ATOM | 1575 | O | GLY | B2086 | 16.305 | 12.537 | 27.475 | 1.00 | 22.83 | O |
| ATOM | 1576 | N | ALA | B2087 | 18.462 | 13.083 | 27.798 | 1.00 | 21.71 | N |
| ATOM | 1577 | CA | ALA | B2087 | 18.938 | 11.848 | 27.172 | 1.00 | 20.63 | C |
| ATOM | 1578 | CB | ALA | B2087 | 20.336 | 12.055 | 26.576 | 1.00 | 21.15 | C |
| ATOM | 1579 | C | ALA | B2087 | 18.955 | 10.671 | 28.157 | 1.00 | 19.81 | C |
| ATOM | 1580 | O | ALA | B2087 | 19.364 | 10.824 | 29.315 | 1.00 | 18.56 | O |
| ATOM | 1581 | N | PRO | B2088 | 18.505 | 9.479 | 27.707 | 1.00 | 18.87 | N |
| ATOM | 1582 | CD | PRO | B2088 | 17.864 | 9.249 | 26.397 | 1.00 | 19.36 | C |
| ATOM | 1583 | CA | PRO | B2088 | 18.461 | 8.258 | 28.530 | 1.00 | 17.76 | C |
| ATOM | 1584 | CB | PRO | B2088 | 17.442 | 7.390 | 27.790 | 1.00 | 18.59 | C |
| ATOM | 1585 | CG | PRO | B2088 | 17.719 | 7.724 | 26.358 | 1.00 | 18.87 | C |
| ATOM | 1586 | C | PRO | B2088 | 19.831 | 7.571 | 28.650 | 1.00 | 16.87 | C |
| ATOM | 1587 | O | PRO | B2088 | 20.593 | 7.559 | 27.688 | 1.00 | 15.25 | O |
| ATOM | 1588 | N | TYR | B2089 | 20.130 | 7.002 | 29.826 | 1.00 | 16.14 | N |
| ATOM | 1589 | CA | TYR | B2089 | 21.405 | 6.311 | 30.078 | 1.00 | 16.26 | C |
| ATOM | 1590 | CB | TYR | B2089 | 22.510 | 7.344 | 30.377 | 1.00 | 17.32 | C |
| ATOM | 1591 | CG | TYR | B2089 | 22.461 | 7.951 | 31.774 | 1.00 | 16.96 | C |
| ATOM | 1592 | CD1 | TYR | B2089 | 23.255 | 7.445 | 32.810 | 1.00 | 18.32 | C |
| ATOM | 1593 | CE1 | TYR | B2089 | 23.217 | 8.007 | 34.098 | 1.00 | 18.36 | C |
| ATOM | 1594 | CD2 | TYR | B2089 | 21.625 | 9.031 | 32.057 | 1.00 | 17.32 | C |
| ATOM | 1595 | CE2 | TYR | B2089 | 21.580 | 9.595 | 33.332 | 1.00 | 18.67 | C |
| ATOM | 1596 | CZ | TYR | B2089 | 22.379 | 9.079 | 34.347 | 1.00 | 18.08 | C |
| ATOM | 1597 | OH | TYR | B2089 | 22.337 | 9.654 | 35.601 | 1.00 | 19.43 | O |
| ATOM | 1598 | C | TYR | B2089 | 21.313 | 5.311 | 31.250 | 1.00 | 16.51 | C |
| ATOM | 1599 | O | TYR | B2089 | 20.453 | 5.455 | 32.127 | 1.00 | 16.59 | O |
| ATOM | 1600 | N | PHE | B2090 | 22.191 | 4.304 | 31.261 | 1.00 | 16.46 | N |
| ATOM | 1601 | CA | PHE | B2090 | 22.207 | 3.292 | 32.338 | 1.00 | 16.71 | C |
| ATOM | 1602 | CB | PHE | B2090 | 22.803 | 1.953 | 31.855 | 1.00 | 15.86 | C |
| ATOM | 1603 | CG | PHE | B2090 | 21.830 | 1.069 | 31.099 | 1.00 | 16.44 | C |
| ATOM | 1604 | CD1 | PHE | B2090 | 21.652 | 1.216 | 29.724 | 1.00 | 16.03 | C |
| ATOM | 1605 | CD2 | PHE | B2090 | 21.089 | 0.094 | 31.770 | 1.00 | 16.46 | C |
| ATOM | 1606 | CE1 | PHE | B2090 | 20.748 | 0.407 | 29.022 | 1.00 | 16.54 | C |
| ATOM | 1607 | CE2 | PHE | B2090 | 20.179 | −0.723 | 31.084 | 1.00 | 16.80 | C |
| ATOM | 1608 | CZ | PHE | B2090 | 20.008 | −0.568 | 29.709 | 1.00 | 16.46 | C |
| ATOM | 1609 | C | PHE | B2090 | 23.027 | 3.748 | 33.550 | 1.00 | 17.25 | C |
| ATOM | 1610 | O | PHE | B2090 | 24.245 | 3.938 | 33.445 | 1.00 | 16.58 | O |
| ATOM | 1611 | N | SER | B2091 | 22.372 | 3.900 | 34.700 | 1.00 | 16.92 | N |
| ATOM | 1612 | CA | SER | B2091 | 23.068 | 4.312 | 35.922 | 1.00 | 18.02 | C |
| ATOM | 1613 | CB | SER | B2091 | 22.148 | 5.177 | 36.802 | 1.00 | 18.33 | C |
| ATOM | 1614 | OG | SER | B2091 | 20.902 | 4.535 | 37.052 | 1.00 | 17.93 | O |
| ATOM | 1615 | C | SER | B2091 | 23.592 | 3.105 | 36.729 | 1.00 | 18.66 | C |
| ATOM | 1616 | O | SER | B2091 | 24.479 | 3.255 | 37.582 | 1.00 | 18.83 | O |
| ATOM | 1617 | N | GLN | B2092 | 23.048 | 1.920 | 36.448 | 1.00 | 18.75 | N |
| ATOM | 1618 | CA | GLN | B2092 | 23.438 | 0.675 | 37.116 | 1.00 | 19.69 | C |
| ATOM | 1619 | CB | GLN | B2092 | 22.526 | 0.377 | 38.313 | 1.00 | 21.62 | C |
| ATOM | 1620 | CG | GLN | B2092 | 22.726 | 1.259 | 39.533 | 1.00 | 25.05 | C |
| ATOM | 1621 | CD | GLN | B2092 | 21.981 | 0.730 | 40.746 | 1.00 | 27.87 | C |
| ATOM | 1622 | OE1 | GLN | B2092 | 20.775 | 0.944 | 40.900 | 1.00 | 29.82 | O |
| ATOM | 1623 | NE2 | GLN | B2092 | 22.696 | 0.018 | 41.611 | 1.00 | 29.02 | N |
| ATOM | 1624 | C | GLN | B2092 | 23.336 | −0.512 | 36.151 | 1.00 | 19.36 | C |
| ATOM | 1625 | O | GLN | B2092 | 22.316 | −0.678 | 35.486 | 1.00 | 18.36 | O |
| ATOM | 1626 | N | ALA | B2093 | 24.383 | −1.336 | 36.094 | 1.00 | 18.90 | N |
| ATOM | 1627 | CA | ALA | B2093 | 24.411 | −2.514 | 35.221 | 1.00 | 19.53 | C |
| ATOM | 1628 | CB | ALA | B2093 | 24.441 | −2.073 | 33.755 | 1.00 | 19.53 | C |
| ATOM | 1629 | C | ALA | B2093 | 25.625 | −3.407 | 35.524 | 1.00 | 20.13 | C |
| ATOM | 1630 | O | ALA | B2093 | 26.641 | −2.928 | 36.024 | 1.00 | 20.09 | O |
| ATOM | 1631 | N | PRO | B2094 | 25.527 | −4.720 | 35.239 | 1.00 | 21.00 | N |
| ATOM | 1632 | CD | PRO | B2094 | 24.303 | −5.472 | 34.892 | 1.00 | 21.48 | C |
| ATOM | 1633 | CA | PRO | B2094 | 26.653 | −5.631 | 35.498 | 1.00 | 21.95 | C |
| ATOM | 1634 | CB | PRO | B2094 | 25.956 | −6.963 | 35.744 | 1.00 | 22.34 | C |
| ATOM | 1635 | CG | PRO | B2094 | 24.808 | −6.905 | 34.739 | 1.00 | 22.42 | C |
| ATOM | 1636 | C | PRO | B2094 | 27.613 | −5.677 | 34.300 | 1.00 | 22.30 | C |
| ATOM | 1637 | O | PRO | B2094 | 27.695 | −6.690 | 33.594 | 1.00 | 22.45 | O |
| ATOM | 1638 | N | PHE | B2095 | 28.331 | −4.572 | 34.083 | 1.00 | 22.56 | N |
| ATOM | 1639 | CA | PHE | B2095 | 29.267 | −4.435 | 32.957 | 1.00 | 23.11 | C |
| ATOM | 1640 | CB | PHE | B2095 | 28.471 | −4.089 | 31.688 | 1.00 | 22.75 | C |
| ATOM | 1641 | CG | PHE | B2095 | 29.307 | −3.979 | 30.439 | 1.00 | 22.31 | C |
| ATOM | 1642 | CD1 | PHE | B2095 | 29.484 | −2.746 | 29.812 | 1.00 | 22.23 | C |
| ATOM | 1643 | CD2 | PHE | B2095 | 29.898 | −5.107 | 29.876 | 1.00 | 22.30 | C |
| ATOM | 1644 | CE1 | PHE | B2095 | 30.237 | −2.639 | 28.640 | 1.00 | 22.73 | C |
| ATOM | 1645 | CE2 | PHE | B2095 | 30.655 | −5.012 | 28.705 | 1.00 | 22.36 | C |
| ATOM | 1646 | CZ | PHE | B2095 | 30.825 | −3.774 | 28.085 | 1.00 | 21.98 | C |
| ATOM | 1647 | C | PHE | B2095 | 30.305 | −3.340 | 33.248 | 1.00 | 23.62 | C |
| ATOM | 1648 | O | PHE | B2095 | 29.965 | −2.284 | 33.778 | 1.00 | 23.96 | O |
| ATOM | 1649 | N | SER | B2096 | 31.561 | −3.584 | 32.884 | 1.00 | 24.19 | N |
| ATOM | 1650 | CA | SER | B2096 | 32.636 | −2.623 | 33.143 | 1.00 | 25.11 | C |
| ATOM | 1651 | CB | SER | B2096 | 33.927 | −3.376 | 33.477 | 1.00 | 26.30 | C |
| ATOM | 1652 | OG | SER | B2096 | 33.722 | −4.306 | 34.523 | 1.00 | 28.33 | O |
| ATOM | 1653 | C | SER | B2096 | 32.954 | −1.591 | 32.050 | 1.00 | 24.46 | C |
| ATOM | 1654 | O | SER | B2096 | 33.606 | −0.583 | 32.332 | 1.00 | 25.13 | O |
| ATOM | 1655 | N | GLY | B2097 | 32.513 | −1.828 | 30.818 | 1.00 | 23.39 | N |
| ATOM | 1656 | CA | GLY | B2097 | 32.818 | −0.891 | 29.739 | 1.00 | 21.60 | C |
| ATOM | 1657 | C | GLY | B2097 | 31.823 | 0.242 | 29.527 | 1.00 | 20.27 | C |
| ATOM | 1658 | O | GLY | B2097 | 31.138 | 0.651 | 30.460 | 1.00 | 20.10 | O |
| ATOM | 1659 | N | LYS | B2098 | 31.756 | 0.758 | 28.299 | 1.00 | 19.44 | N |
| ATOM | 1660 | CA | LYS | B2098 | 30.822 | 1.836 | 27.954 | 1.00 | 17.90 | C |
| ATOM | 1661 | CB | LYS | B2098 | 31.472 | 2.822 | 26.978 | 1.00 | 20.19 | C |
| ATOM | 1662 | CG | LYS | B2098 | 32.604 | 3.651 | 27.561 | 1.00 | 21.96 | C |
| ATOM | 1663 | CD | LYS | B2098 | 33.055 | 4.682 | 26.543 | 1.00 | 24.18 | C |
| ATOM | 1664 | CE | LYS | B2098 | 34.115 | 5.612 | 27.103 | 1.00 | 25.70 | C |
| ATOM | 1665 | NZ | LYS | B2098 | 34.538 | 6.626 | 26.087 | 1.00 | 27.22 | N |
| ATOM | 1666 | C | LYS | B2098 | 29.561 | 1.256 | 27.309 | 1.00 | 16.51 | C |
| ATOM | 1667 | O | LYS | B2098 | 29.647 | 0.298 | 26.543 | 1.00 | 14.73 | O |
| ATOM | 1668 | N | ILE | B2099 | 28.401 | 1.844 | 27.621 | 1.00 | 15.59 | N |
| ATOM | 1669 | CA | ILE | B2099 | 27.107 | 1.395 | 27.086 | 1.00 | 15.02 | C |
| ATOM | 1670 | CB | ILE | B2099 | 26.144 | 0.954 | 28.236 | 1.00 | 15.10 | C |
| ATOM | 1671 | CG2 | ILE | B2099 | 24.823 | 0.429 | 27.647 | 1.00 | 16.11 | C |
| ATOM | 1672 | CG1 | ILE | B2099 | 26.790 | −0.138 | 29.095 | 1.00 | 15.18 | C |
| ATOM | 1673 | CD1 | ILE | B2099 | 25.994 | −0.462 | 30.363 | 1.00 | 16.41 | C |
| ATOM | 1674 | C | ILE | B2099 | 26.406 | 2.533 | 26.308 | 1.00 | 14.43 | C |
| ATOM | 1675 | O | ILE | B2099 | 26.039 | 3.551 | 26.900 | 1.00 | 14.51 | O |
| ATOM | 1676 | N | TRP | B2100 | 26.207 | 2.359 | 25.000 | 1.00 | 13.72 | N |
| ATOM | 1677 | CA | TRP | B2100 | 25.547 | 3.381 | 24.173 | 1.00 | 13.62 | C |
| ATOM | 1678 | CB | TRP | B2100 | 26.342 | 3.558 | 22.864 | 1.00 | 13.58 | C |
| ATOM | 1679 | CG | TRP | B2100 | 27.817 | 3.938 | 23.093 | 1.00 | 13.89 | C |
| ATOM | 1680 | CD2 | TRP | B2100 | 28.334 | 5.257 | 23.347 | 1.00 | 13.95 | C |
| ATOM | 1681 | CE2 | TRP | B2100 | 29.726 | 5.126 | 23.597 | 1.00 | 14.82 | C |
| ATOM | 1682 | CE3 | TRP | B2100 | 27.755 | 6.535 | 23.395 | 1.00 | 14.46 | C |
| ATOM | 1683 | CD1 | TRP | B2100 | 28.890 | 3.085 | 23.187 | 1.00 | 14.49 | C |
| ATOM | 1684 | NE1 | TRP | B2100 | 30.041 | 3.795 | 23.492 | 1.00 | 14.89 | N |
| ATOM | 1685 | CZ2 | TRP | B2100 | 30.546 | 6.230 | 23.895 | 1.00 | 15.47 | C |
| ATOM | 1686 | CZ3 | TRP | B2100 | 28.577 | 7.640 | 23.697 | 1.00 | 15.28 | C |
| ATOM | 1687 | CH2 | TRP | B2100 | 29.955 | 7.472 | 23.942 | 1.00 | 14.88 | C |
| ATOM | 1688 | C | TRP | B2100 | 24.071 | 2.998 | 23.886 | 1.00 | 13.84 | C |
| ATOM | 1689 | O | TRP | B2100 | 23.816 | 1.995 | 23.214 | 1.00 | 13.89 | O |
| ATOM | 1690 | N | LEU | B2101 | 23.120 | 3.808 | 24.379 | 1.00 | 13.08 | N |
| ATOM | 1691 | CA | LEU | B2101 | 21.657 | 3.557 | 24.239 | 1.00 | 12.41 | C |
| ATOM | 1692 | CB | LEU | B2101 | 21.059 | 3.267 | 25.634 | 1.00 | 13.29 | C |
| ATOM | 1693 | CG | LEU | B2101 | 19.524 | 3.353 | 25.825 | 1.00 | 12.98 | C |
| ATOM | 1694 | CD1 | LEU | B2101 | 18.889 | 2.043 | 25.355 | 1.00 | 12.28 | C |
| ATOM | 1695 | CD2 | LEU | B2101 | 19.156 | 3.601 | 27.303 | 1.00 | 12.42 | C |
| ATOM | 1696 | C | LEU | B2101 | 20.796 | 4.674 | 23.602 | 1.00 | 11.67 | C |
| ATOM | 1697 | O | LEU | B2101 | 21.063 | 5.858 | 23.811 | 1.00 | 11.61 | O |
| ATOM | 1698 | N | SER | B2102 | 19.760 | 4.287 | 22.844 | 1.00 | 11.90 | N |
| ATOM | 1699 | CA | SER | B2102 | 18.799 | 5.237 | 22.237 | 1.00 | 11.37 | C |
| ATOM | 1700 | CB | SER | B2102 | 19.212 | 5.616 | 20.810 | 1.00 | 11.79 | C |
| ATOM | 1701 | OG | SER | B2102 | 18.405 | 6.679 | 20.308 | 1.00 | 9.95 | O |
| ATOM | 1702 | C | SER | B2102 | 17.371 | 4.634 | 22.219 | 1.00 | 11.48 | C |
| ATOM | 1703 | O | SER | B2102 | 17.220 | 3.421 | 22.026 | 1.00 | 11.63 | O |
| ATOM | 1704 | N | ILE | B2103 | 16.351 | 5.482 | 22.425 | 1.00 | 10.47 | N |
| ATOM | 1705 | CA | ILE | B2103 | 14.912 | 5.096 | 22.465 | 1.00 | 10.97 | C |
| ATOM | 1706 | CB | ILE | B2103 | 14.337 | 5.250 | 23.934 | 1.00 | 11.04 | C |
| ATOM | 1707 | CG2 | ILE | B2103 | 12.852 | 4.835 | 23.981 | 1.00 | 11.15 | C |
| ATOM | 1708 | CG1 | ILE | B2103 | 15.136 | 4.390 | 24.924 | 1.00 | 10.41 | C |
| ATOM | 1709 | CD1 | ILE | B2103 | 14.781 | 4.658 | 26.424 | 1.00 | 10.59 | C |
| ATOM | 1710 | C | ILE | B2103 | 14.042 | 5.995 | 21.536 | 1.00 | 11.56 | C |
| ATOM | 1711 | O | ILE | B2103 | 14.327 | 7.184 | 21.393 | 1.00 | 11.36 | O |
| ATOM | 1712 | N | SER | B2104 | 12.994 | 5.435 | 20.915 | 1.00 | 13.13 | N |
| ATOM | 1713 | CA | SER | B2104 | 12.073 | 6.214 | 20.054 | 1.00 | 14.88 | C |
| ATOM | 1714 | CB | SER | B2104 | 12.582 | 6.263 | 18.603 | 1.00 | 15.92 | C |
| ATOM | 1715 | OG | SER | B2104 | 11.787 | 7.132 | 17.789 | 1.00 | 16.21 | O |
| ATOM | 1716 | C | SER | B2104 | 10.652 | 5.617 | 20.070 | 1.00 | 15.87 | C |
| ATOM | 1717 | O | SER | B2104 | 10.485 | 4.418 | 20.297 | 1.00 | 15.32 | O |
| ATOM | 1718 | N | HIS | B2105 | 9.623 | 6.429 | 19.822 | 1.00 | 17.79 | N |
| ATOM | 1719 | CA | HIS | B2105 | 8.266 | 5.872 | 19.833 | 1.00 | 19.31 | C |
| ATOM | 1720 | CB | HIS | B2105 | 7.683 | 5.921 | 21.249 | 1.00 | 21.32 | C |
| ATOM | 1721 | CG | HIS | B2105 | 7.405 | 7.306 | 21.746 | 1.00 | 23.02 | C |
| ATOM | 1722 | CD2 | HIS | B2105 | 6.279 | 8.058 | 21.704 | 1.00 | 25.09 | C |
| ATOM | 1723 | ND1 | HIS | B2105 | 8.363 | 8.086 | 22.356 | 1.00 | 25.22 | N |
| ATOM | 1724 | CE1 | HIS | B2105 | 7.841 | 9.260 | 22.669 | 1.00 | 25.65 | C |
| ATOM | 1725 | NE2 | HIS | B2105 | 6.577 | 9.269 | 22.285 | 1.00 | 26.41 | N |
| ATOM | 1726 | C | HIS | B2105 | 7.237 | 6.486 | 18.870 | 1.00 | 20.14 | C |
| ATOM | 1727 | O | HIS | B2105 | 7.453 | 7.560 | 18.305 | 1.00 | 18.58 | O |
| ATOM | 1728 | N | THR | B2106 | 6.128 | 5.763 | 18.687 | 1.00 | 20.92 | N |
| ATOM | 1729 | CA | THR | B2106 | 5.008 | 6.208 | 17.852 | 1.00 | 23.37 | C |
| ATOM | 1730 | CB | THR | B2106 | 4.758 | 5.302 | 16.612 | 1.00 | 23.62 | C |
| ATOM | 1731 | OG1 | THR | B2106 | 4.237 | 4.035 | 17.039 | 1.00 | 24.37 | O |
| ATOM | 1732 | CG2 | THR | B2106 | 6.043 | 5.092 | 15.820 | 1.00 | 23.72 | C |
| ATOM | 1733 | C | THR | B2106 | 3.766 | 6.106 | 18.735 | 1.00 | 23.79 | C |
| ATOM | 1734 | O | THR | B2106 | 3.867 | 5.875 | 19.937 | 1.00 | 24.63 | O |
| ATOM | 1735 | N | ASP | B2107 | 2.591 | 6.255 | 18.138 | 1.00 | 24.86 | N |
| ATOM | 1736 | CA | ASP | B2107 | 1.355 | 6.176 | 18.906 | 1.00 | 24.83 | C |
| ATOM | 1737 | CB | ASP | B2107 | 0.189 | 6.691 | 18.059 | 0.05 | 24.72 | C |
| ATOM | 1738 | CG | ASP | B2107 | −0.652 | 7.716 | 18.790 | 0.05 | 24.62 | C |
| ATOM | 1739 | OD1 | ASP | B2107 | −1.227 | 7.372 | 19.844 | 0.05 | 24.64 | O |
| ATOM | 1740 | OD2 | ASP | B2107 | −0.738 | 8.867 | 18.312 | 0.05 | 24.51 | O |
| ATOM | 1741 | C | ASP | B2107 | 1.043 | 4.763 | 19.403 | 1.00 | 24.92 | C |
| ATOM | 1742 | O | ASP | B2107 | 0.368 | 4.598 | 20.421 | 1.00 | 26.07 | O |
| ATOM | 1743 | N | GLN | B2108 | 1.535 | 3.745 | 18.701 | 1.00 | 24.68 | N |
| ATOM | 1744 | CA | GLN | B2108 | 1.255 | 2.361 | 19.086 | 1.00 | 24.22 | C |
| ATOM | 1745 | CB | GLN | B2108 | 0.522 | 1.652 | 17.939 | 1.00 | 25.96 | C |
| ATOM | 1746 | CG | GLN | B2108 | −0.753 | 2.352 | 17.462 | 1.00 | 27.90 | C |
| ATOM | 1747 | CD | GLN | B2108 | −1.328 | 1.732 | 16.189 | 1.00 | 29.69 | C |
| ATOM | 1748 | OE1 | GLN | B2108 | −2.371 | 1.073 | 16.215 | 1.00 | 31.19 | O |
| ATOM | 1749 | NE2 | GLN | B2108 | −0.641 | 1.940 | 15.069 | 1.00 | 30.55 | N |
| ATOM | 1750 | C | GLN | B2108 | 2.450 | 1.489 | 19.517 | 1.00 | 23.66 | C |
| ATOM | 1751 | O | GLN | B2108 | 2.246 | 0.490 | 20.216 | 1.00 | 22.94 | O |
| ATOM | 1752 | N | PHE | B2109 | 3.672 | 1.854 | 19.115 | 1.00 | 22.58 | N |
| ATOM | 1753 | CA | PHE | B2109 | 4.869 | 1.058 | 19.450 | 1.00 | 22.19 | C |
| ATOM | 1754 | CB | PHE | B2109 | 5.440 | 0.378 | 18.187 | 1.00 | 23.42 | C |
| ATOM | 1755 | CG | PHE | B2109 | 4.409 | −0.256 | 17.287 | 1.00 | 25.28 | C |
| ATOM | 1756 | CD1 | PHE | B2109 | 3.830 | 0.469 | 16.248 | 1.00 | 26.47 | C |
| ATOM | 1757 | CD2 | PHE | B2109 | 4.038 | −1.586 | 17.459 | 1.00 | 26.35 | C |
| ATOM | 1758 | CE1 | PHE | B2109 | 2.894 | −0.123 | 15.386 | 1.00 | 26.84 | C |
| ATOM | 1759 | CE2 | PHE | B2109 | 3.106 | −2.190 | 16.607 | 1.00 | 27.41 | C |
| ATOM | 1760 | CZ | PHE | B2109 | 2.534 | −1.454 | 15.567 | 1.00 | 27.07 | C |
| ATOM | 1761 | C | PHE | B2109 | 6.031 | 1.852 | 20.088 | 1.00 | 20.94 | C |
| ATOM | 1762 | O | PHE | B2109 | 6.034 | 3.082 | 20.086 | 1.00 | 20.73 | O |
| ATOM | 1763 | N | VAL | B2110 | 7.020 | 1.128 | 20.619 | 1.00 | 20.20 | N |
| ATOM | 1764 | CA | VAL | B2110 | 8.225 | 1.737 | 21.210 | 1.00 | 18.93 | C |
| ATOM | 1765 | CB | VAL | B2110 | 8.115 | 1.867 | 22.761 | 1.00 | 19.35 | C |
| ATOM | 1766 | CG1 | VAL | B2110 | 7.968 | 0.496 | 23.404 | 1.00 | 21.12 | C |
| ATOM | 1767 | CG2 | VAL | B2110 | 9.339 | 2.595 | 23.316 | 1.00 | 19.95 | C |
| ATOM | 1768 | C | VAL | B2110 | 9.423 | 0.846 | 20.845 | 1.00 | 18.01 | C |
| ATOM | 1769 | O | VAL | B2110 | 9.295 | −0.380 | 20.827 | 1.00 | 17.84 | O |
| ATOM | 1770 | N | THR | B2111 | 10.576 | 1.450 | 20.548 | 1.00 | 16.67 | N |
| ATOM | 1771 | CA | THR | B2111 | 11.768 | 0.678 | 20.170 | 1.00 | 15.96 | C |
| ATOM | 1772 | CB | THR | B2111 | 11.950 | 0.693 | 18.628 | 1.00 | 17.10 | C |
| ATOM | 1773 | OG1 | THR | B2111 | 13.011 | −0.201 | 18.258 | 1.00 | 21.79 | O |
| ATOM | 1774 | CG2 | THR | B2111 | 12.283 | 2.096 | 18.141 | 1.00 | 17.28 | C |
| ATOM | 1775 | C | THR | B2111 | 13.049 | 1.203 | 20.839 | 1.00 | 14.55 | C |
| ATOM | 1776 | O | THR | B2111 | 13.122 | 2.380 | 21.194 | 1.00 | 12.59 | O |
| ATOM | 1777 | N | ALA | B2112 | 14.044 | 0.326 | 21.005 | 1.00 | 13.42 | N |
| ATOM | 1778 | CA | ALA | B2112 | 15.325 | 0.680 | 21.644 | 1.00 | 12.71 | C |
| ATOM | 1779 | CB | ALA | B2112 | 15.247 | 0.379 | 23.145 | 1.00 | 12.85 | C |
| ATOM | 1780 | C | ALA | B2112 | 16.516 | −0.078 | 21.024 | 1.00 | 12.80 | C |
| ATOM | 1781 | O | ALA | B2112 | 16.348 | −1.196 | 20.535 | 1.00 | 13.45 | O |
| ATOM | 1782 | N | SER | B2113 | 17.709 | 0.527 | 21.060 | 1.00 | 12.54 | N |
| ATOM | 1783 | CA | SER | B2113 | 18.937 | −0.077 | 20.498 | 1.00 | 12.67 | C |
| ATOM | 1784 | CB | SER | B2113 | 19.222 | 0.539 | 19.115 | 1.00 | 12.72 | C |
| ATOM | 1785 | OG | SER | B2113 | 20.402 | 0.007 | 18.523 | 1.00 | 13.88 | O |
| ATOM | 1786 | C | SER | B2113 | 20.152 | 0.142 | 21.443 | 1.00 | 11.86 | C |
| ATOM | 1787 | O | SER | B2113 | 20.326 | 1.247 | 21.973 | 1.00 | 12.14 | O |
| ATOM | 1788 | N | VAL | B2114 | 20.967 | −0.905 | 21.636 | 1.00 | 10.76 | N |
| ATOM | 1789 | CA | VAL | B2114 | 22.157 | −0.885 | 22.526 | 1.00 | 11.02 | C |
| ATOM | 1790 | CB | VAL | B2114 | 21.912 | −1.748 | 23.810 | 1.00 | 10.07 | C |
| ATOM | 1791 | CG1 | VAL | B2114 | 23.204 | −1.874 | 24.619 | 1.00 | 12.63 | C |
| ATOM | 1792 | CG2 | VAL | B2114 | 20.808 | −1.124 | 24.689 | 1.00 | 11.23 | C |
| ATOM | 1793 | C | VAL | B2114 | 23.451 | −1.438 | 21.880 | 1.00 | 11.08 | C |
| ATOM | 1794 | O | VAL | B2114 | 23.408 | −2.477 | 21.204 | 1.00 | 10.49 | O |
| ATOM | 1795 | N | ILE | B2115 | 24.584 | −0.753 | 22.093 | 1.00 | 11.85 | N |
| ATOM | 1796 | CA | ILE | B2115 | 25.905 | −1.198 | 21.585 | 1.00 | 11.88 | C |
| ATOM | 1797 | CB | ILE | B2115 | 26.485 | −0.236 | 20.492 | 1.00 | 12.44 | C |
| ATOM | 1798 | CG2 | ILE | B2115 | 27.841 | −0.756 | 19.999 | 1.00 | 12.78 | C |
| ATOM | 1799 | CG1 | ILE | B2115 | 25.529 | −0.134 | 19.300 | 1.00 | 12.56 | C |
| ATOM | 1800 | CD1 | ILE | B2115 | 25.889 | 0.986 | 18.331 | 1.00 | 12.68 | C |
| ATOM | 1801 | C | ILE | B2115 | 26.883 | −1.197 | 22.782 | 1.00 | 12.95 | C |
| ATOM | 1802 | O | ILE | B2115 | 26.966 | −0.197 | 23.507 | 1.00 | 13.03 | O |
| ATOM | 1803 | N | LEU | B2116 | 27.600 | −2.306 | 22.994 | 1.00 | 12.77 | N |
| ATOM | 1804 | CA | LEU | B2116 | 28.559 | −2.432 | 24.115 | 1.00 | 14.04 | C |
| ATOM | 1805 | CB | LEU | B2116 | 28.353 | −3.785 | 24.830 | 1.00 | 13.62 | C |
| ATOM | 1806 | CG | LEU | B2116 | 26.929 | −4.093 | 25.349 | 1.00 | 14.05 | C |
| ATOM | 1807 | CD1 | LEU | B2116 | 26.866 | −5.489 | 25.971 | 1.00 | 15.37 | C |
| ATOM | 1808 | CD2 | LEU | B2116 | 26.520 | −3.055 | 26.384 | 1.00 | 14.15 | C |
| ATOM | 1809 | C | LEU | B2116 | 30.020 | −2.296 | 23.630 | 1.00 | 14.81 | C |
| ATOM | 1810 | O | LEU | B2116 | 30.381 | −2.839 | 22.582 | 1.00 | 14.52 | O |
| ATOM | 1811 | N | GLU | B2117 | 30.858 | −1.605 | 24.409 | 1.00 | 16.40 | N |
| ATOM | 1812 | CA | GLU | B2117 | 32.255 | −1.353 | 24.027 | 1.00 | 18.58 | C |
| ATOM | 1813 | CB | GLU | B2117 | 32.344 | 0.062 | 23.426 | 1.00 | 18.84 | C |
| ATOM | 1814 | CG | GLU | B2117 | 33.737 | 0.570 | 23.061 | 1.00 | 21.74 | C |
| ATOM | 1815 | CD | GLU | B2117 | 33.703 | 1.977 | 22.450 | 1.00 | 23.34 | C |
| ATOM | 1816 | OE1 | GLU | B2117 | 33.161 | 2.908 | 23.098 | 1.00 | 22.55 | O |
| ATOM | 1817 | OE2 | GLU | B2117 | 34.221 | 2.153 | 21.322 | 1.00 | 24.09 | O |
| ATOM | 1818 | C | GLU | B2117 | 33.247 | −1.495 | 25.196 | 1.00 | 20.32 | C |
| ATOM | 1819 | O | GLU | B2117 | 32.989 | −1.010 | 26.303 | 1.00 | 18.78 | O |
| ATOM | 1820 | N | GLU | B2118 | 34.384 | −2.141 | 24.931 | 1.00 | 22.46 | N |
| ATOM | 1821 | CA | GLU | B2118 | 35.413 | −2.366 | 25.949 | 1.00 | 26.31 | C |
| ATOM | 1822 | CB | GLU | B2118 | 35.364 | −3.830 | 26.415 | 1.00 | 28.48 | C |
| ATOM | 1823 | CG | GLU | B2118 | 36.185 | −4.151 | 27.657 | 1.00 | 31.58 | C |
| ATOM | 1824 | CD | GLU | B2118 | 35.479 | −3.793 | 28.952 | 1.00 | 33.18 | C |
| ATOM | 1825 | OE1 | GLU | B2118 | 34.479 | −4.464 | 29.295 | 1.00 | 34.62 | O |
| ATOM | 1826 | OE2 | GLU | B2118 | 35.925 | −2.839 | 29.628 | 1.00 | 34.75 | O |
| ATOM | 1827 | C | GLU | B2118 | 36.820 | −2.039 | 25.435 | 1.00 | 27.70 | C |
| ATOM | 1828 | O | GLU | B2118 | 36.958 | −1.559 | 24.285 | 1.00 | 28.17 | O |
| ATOM | 1829 | OXT | GLU | B2118 | 37.784 | −2.267 | 26.199 | 1.00 | 29.81 | O |
| TER | 1830 | | GLU | B2118 | | | | | | |
| ATOM | 1831 | CB | MET | C3003 | 38.407 | 0.566 | 12.507 | 1.00 | 29.11 | C |
| ATOM | 1832 | CG | MET | C3003 | 38.953 | −0.372 | 11.436 | 1.00 | 32.10 | C |
| ATOM | 1833 | SD | MET | C3003 | 39.971 | 0.467 | 10.186 | 1.00 | 36.76 | S |
| ATOM | 1834 | CE | MET | C3003 | 41.535 | 0.656 | 11.078 | 1.00 | 35.20 | C |
| ATOM | 1835 | C | MET | C3003 | 36.229 | −0.625 | 12.723 | 1.00 | 24.49 | C |
| ATOM | 1836 | O | MET | C3003 | 36.251 | −1.743 | 12.216 | 1.00 | 24.23 | O |
| ATOM | 1837 | N | MET | C3003 | 38.104 | −1.203 | 14.236 | 1.00 | 26.20 | N |
| ATOM | 1838 | CA | MET | C3003 | 37.432 | −0.097 | 13.493 | 1.00 | 26.22 | C |
| ATOM | 1839 | N | ILE | C3004 | 35.182 | 0.190 | 12.648 | 1.00 | 22.87 | N |
| ATOM | 1840 | CA | ILE | C3004 | 33.960 | −0.165 | 11.933 | 1.00 | 21.52 | C |
| ATOM | 1841 | CB | ILE | C3004 | 32.800 | 0.781 | 12.355 | 1.00 | 21.32 | C |
| ATOM | 1842 | CG2 | ILE | C3004 | 31.535 | 0.469 | 11.557 | 1.00 | 21.05 | C |
| ATOM | 1843 | CG1 | ILE | C3004 | 32.547 | 0.638 | 13.864 | 1.00 | 20.83 | C |
| ATOM | 1844 | CD1 | ILE | C3004 | 31.537 | 1.615 | 14.443 | 1.00 | 20.25 | C |
| ATOM | 1845 | C | ILE | C3004 | 34.199 | −0.043 | 10.422 | 1.00 | 21.57 | C |
| ATOM | 1846 | O | ILE | C3004 | 34.938 | 0.848 | 9.979 | 1.00 | 21.16 | O |
| ATOM | 1847 | N | VAL | C3005 | 33.610 | −0.952 | 9.638 | 1.00 | 20.59 | N |
| ATOM | 1848 | CA | VAL | C3005 | 33.749 | −0.906 | 8.182 | 1.00 | 20.63 | C |
| ATOM | 1849 | CB | VAL | C3005 | 34.642 | −2.068 | 7.628 | 1.00 | 21.47 | C |
| ATOM | 1850 | CG1 | VAL | C3005 | 36.083 | −1.890 | 8.090 | 1.00 | 21.00 | C |
| ATOM | 1851 | CG2 | VAL | C3005 | 34.109 | −3.413 | 8.076 | 1.00 | 23.00 | C |
| ATOM | 1852 | C | VAL | C3005 | 32.413 | −0.901 | 7.422 | 1.00 | 19.52 | C |
| ATOM | 1853 | O | VAL | C3005 | 32.411 | −0.880 | 6.192 | 1.00 | 19.11 | O |
| ATOM | 1854 | N | GLY | C3006 | 31.289 | −0.906 | 8.149 | 1.00 | 18.23 | N |
| ATOM | 1855 | CA | GLY | C3006 | 29.974 | −0.880 | 7.508 | 1.00 | 17.17 | C |
| ATOM | 1856 | C | GLY | C3006 | 28.797 | −0.986 | 8.482 | 1.00 | 16.61 | C |
| ATOM | 1857 | O | GLY | C3006 | 28.963 | −1.526 | 9.579 | 1.00 | 16.76 | O |
| ATOM | 1858 | N | HIS | C3007 | 27.620 | −0.477 | 8.093 | 1.00 | 15.51 | N |
| ATOM | 1859 | CA | HIS | C3007 | 26.400 | −0.531 | 8.935 | 1.00 | 15.40 | C |
| ATOM | 1860 | CB | HIS | C3007 | 26.418 | 0.624 | 9.953 | 1.00 | 15.15 | C |
| ATOM | 1861 | CG | HIS | C3007 | 25.191 | 0.709 | 10.821 | 1.00 | 14.61 | C |
| ATOM | 1862 | C02 | HIS | C3007 | 24.430 | 1.772 | 11.180 | 1.00 | 14.84 | C |
| ATOM | 1863 | ND1 | HIS | C3007 | 24.668 | −0.377 | 11.493 | 1.00 | 15.01 | N |
| ATOM | 1864 | CE1 | HIS | C3007 | 23.639 | 0.013 | 12.229 | 1.00 | 15.56 | C |
| ATOM | 1865 | NE2 | HIS | C3007 | 23.475 | 1.313 | 12.058 | 1.00 | 15.15 | N |
| ATOM | 1866 | C | HIS | C3007 | 25.086 | −0.476 | 8.115 | 1.00 | 14.78 | C |
| ATOM | 1867 | O | HIS | C3007 | 24.933 | 0.372 | 7.228 | 1.00 | 14.49 | O |
| ATOM | 1868 | N | GLY | C3008 | 24.145 | −1.376 | 8.418 | 1.00 | 14.82 | N |
| ATOM | 1869 | CA | GLY | C3008 | 22.867 | −1.390 | 7.709 | 1.00 | 14.90 | C |
| ATOM | 1870 | C | GLY | C3008 | 21.668 | −1.918 | 8.495 | 1.00 | 15.74 | C |
| ATOM | 1871 | O | GLY | C3008 | 21.835 | −2.757 | 9.391 | 1.00 | 14.75 | O |
| ATOM | 1872 | N | ILE | C3009 | 20.465 | −1.425 | 8.166 | 1.00 | 15.25 | N |
| ATOM | 1873 | CA | ILE | C3009 | 19.211 | −1.847 | 8.823 | 1.00 | 15.66 | C |
| ATOM | 1874 | CB | ILE | C3009 | 18.692 | −0.769 | 9.837 | 1.00 | 14.95 | C |
| ATOM | 1875 | CG2 | ILE | C3009 | 19.789 | −0.421 | 10.873 | 1.00 | 15.05 | C |
| ATOM | 1876 | CG1 | ILE | C3009 | 18.261 | 0.492 | 9.079 | 1.00 | 14.33 | C |
| ATOM | 1877 | CD1 | ILE | C3009 | 17.632 | 1.555 | 9.956 | 1.00 | 14.63 | C |
| ATOM | 1878 | C | ILE | C3009 | 18.090 | −2.102 | 7.780 | 1.00 | 16.20 | C |
| ATOM | 1879 | O | ILE | C3009 | 18.221 | −1.696 | 6.619 | 1.00 | 16.14 | O |
| ATOM | 1880 | N | ASP | C3010 | 17.004 | −2.762 | 8.204 | 1.00 | 16.80 | N |
| ATOM | 1881 | CA | ASP | C3010 | 15.853 | −3.080 | 7.330 | 1.00 | 17.32 | C |
| ATOM | 1882 | CB | ASP | C3010 | 16.130 | −4.365 | 6.527 | 1.00 | 18.68 | C |
| ATOM | 1883 | CG | ASP | C3010 | 14.977 | −4.741 | 5.579 | 1.00 | 18.99 | C |
| ATOM | 1884 | OD1 | ASP | C3010 | 14.852 | −4.105 | 4.518 | 1.00 | 19.45 | O |
| ATOM | 1885 | OD2 | ASP | C3010 | 14.193 | −5.667 | 5.896 | 1.00 | 19.42 | O |
| ATOM | 1886 | C | ASP | C3010 | 14.549 | −3.290 | 8.120 | 1.00 | 18.11 | C |
| ATOM | 1887 | O | ASP | C3010 | 14.580 | −3.711 | 9.283 | 1.00 | 17.23 | O |
| ATOM | 1888 | N | ILE | C3011 | 13.411 | −2.978 | 7.488 | 1.00 | 17.97 | N |
| ATOM | 1889 | CA | ILE | C3011 | 12.082 | −3.191 | 8.081 | 1.00 | 18.54 | C |
| ATOM | 1890 | CB | ILE | C3011 | 11.444 | −1.876 | 8.656 | 1.00 | 18.66 | C |
| ATOM | 1891 | CG2 | ILE | C3011 | 11.363 | −0.776 | 7.579 | 1.00 | 19.04 | C |
| ATOM | 1892 | CG1 | ILE | C3011 | 10.048 | −2.185 | 9.211 | 1.00 | 19.19 | C |
| ATOM | 1893 | CG1 | ILE | C3011 | 9.421 | −1.049 | 9.999 | 1.00 | 19.08 | C |
| ATOM | 1894 | C | ILE | C3011 | 11.207 | −3.762 | 6.950 | 1.00 | 18.75 | C |
| ATOM | 1895 | O | ILE | C3011 | 11.286 | −3.288 | 5.825 | 1.00 | 18.39 | O |
| ATOM | 1896 | N | GLU | C3012 | 10.389 | −4.774 | 7.253 | 1.00 | 19.66 | N |
| ATOM | 1897 | CA | GLU | C3012 | 9.544 | −5.433 | 6.246 | 1.00 | 20.80 | C |
| ATOM | 1898 | CB | GLU | C3012 | 10.290 | −6.663 | 5.706 | 1.00 | 22.15 | C |
| ATOM | 1899 | CG | GLU | C3012 | 9.468 | −7.566 | 4.774 | 1.00 | 23.49 | C |
| ATOM | 1900 | CD | GLU | C3012 | 9.692 | −7.274 | 3.300 | 1.00 | 24.70 | C |
| ATOM | 1901 | OE1 | GLU | C3012 | 10.294 | −6.228 | 2.973 | 1.00 | 24.77 | O |
| ATOM | 1902 | OE2 | GLU | C3012 | 9.258 | −8.095 | 2.460 | 1.00 | 25.50 | O |
| ATOM | 1903 | C | GLU | C3012 | 8.160 | −5.870 | 6.759 | 1.00 | 21.36 | C |
| ATOM | 1904 | O | GLU | C3012 | 8.049 | −6.431 | 7.848 | 1.00 | 20.37 | O |
| ATOM | 1905 | N | GLU | C3013 | 7.111 | −5.614 | 5.971 | 1.00 | 22.03 | N |
| ATOM | 1906 | CA | GLU | C3013 | 5.743 | −6.004 | 6.345 | 1.00 | 23.43 | C |
| ATOM | 1907 | CB | GLU | C3013 | 4.703 | −5.207 | 5.534 | 1.00 | 24.60 | C |
| ATOM | 1908 | CG | GLU | C3013 | 4.706 | −3.683 | 5.716 | 1.00 | 26.52 | C |
| ATOM | 1909 | CD | GLU | C3013 | 4.063 | −3.212 | 7.018 | 1.00 | 28.49 | C |
| ATOM | 1910 | OE1 | GLU | C3013 | 3.071 | −3.834 | 7.467 | 1.00 | 28.55 | O |
| ATOM | 1911 | OE2 | GLU | C3013 | 4.533 | −2.192 | 7.579 | 1.00 | 28.72 | O |
| ATOM | 1912 | C | GLU | C3013 | 5.517 | −7.501 | 6.075 | 1.00 | 23.35 | C |
| ATOM | 1913 | O | GLU | C3013 | 5.931 | −8.012 | 5.032 | 1.00 | 23.44 | O |
| ATOM | 1914 | N | LEU | C3014 | 4.855 | −8.198 | 7.000 | 1.00 | 23.78 | N |
| ATOM | 1915 | CA | LEU | C3014 | 4.578 | −9.627 | 6.818 | 1.00 | 24.31 | C |
| ATOM | 1916 | CB | LEU | C3014 | 3.922 | −10.227 | 8.071 | 1.00 | 24.95 | C |
| ATOM | 1917 | CG | LEU | C3014 | 4.664 | −10.149 | 9.417 | 1.00 | 27.00 | C |
| ATOM | 1918 | CD1 | LEU | C3014 | 3.957 | −11.044 | 10.423 | 1.00 | 27.46 | C |
| ATOM | 1919 | CD2 | LEU | C3014 | 6.118 | −10.589 | 9.258 | 1.00 | 26.69 | C |
| ATOM | 1920 | C | LEU | C3014 | 3.660 | −9.867 | 5.608 | 1.00 | 24.29 | C |
| ATOM | 1921 | O | LEU | C3014 | 3.680 | −10.939 | 5.004 | 1.00 | 23.68 | O |
| ATOM | 1922 | N | ALA | C3015 | 2.857 | −8.868 | 5.262 | 1.00 | 24.50 | N |
| ATOM | 1923 | CA | ALA | C3015 | 1.946 | −8.984 | 4.120 | 1.00 | 24.91 | C |
| ATOM | 1924 | CB | ALA | C3015 | 1.082 | −7.732 | 4.016 | 1.00 | 25.04 | C |
| ATOM | 1925 | C | ALA | C3015 | 2.691 | −9.220 | 2.803 | 1.00 | 25.57 | C |
| ATOM | 1926 | O | ALA | C3015 | 2.215 | −9.962 | 1.940 | 1.00 | 25.88 | O |
| ATOM | 1927 | N | SER | C3016 | 3.857 | −8.592 | 2.648 | 1.00 | 25.61 | N |
| ATOM | 1928 | CA | SER | C3016 | 4.658 | −8.737 | 1.432 | 1.00 | 25.91 | C |
| ATOM | 1929 | CB | SER | C3016 | 5.829 | −7.742 | 1.441 | 1.00 | 26.86 | C |
| ATOM | 1930 | OG | SER | C3016 | 5.385 | −6.401 | 1.589 | 1.00 | 27.09 | O |
| ATOM | 1931 | C | SER | C3016 | 5.204 | −10.159 | 1.275 | 1.00 | 26.38 | C |
| ATOM | 1932 | O | SER | C3016 | 5.276 | −10.690 | 0.161 | 1.00 | 25.43 | O |
| ATOM | 1933 | N | ILE | C3017 | 5.603 | −10.768 | 2.390 | 1.00 | 26.46 | N |
| ATOM | 1934 | CA | ILE | C3017 | 6.128 | −12.131 | 2.365 | 1.00 | 26.86 | C |
| ATOM | 1935 | CB | ILE | C3017 | 6.839 | −12.474 | 3.706 | 1.00 | 26.57 | C |
| ATOM | 1936 | CG2 | ILE | C3017 | 7.306 | −13.925 | 3.709 | 1.00 | 26.15 | C |
| ATOM | 1937 | CG1 | ILE | C3017 | 8.029 | −11.526 | 3.923 | 1.00 | 26.30 | C |
| ATOM | 1938 | CD1 | ILE | C3017 | 9.102 | −11.612 | 2.842 | 1.00 | 26.29 | C |
| ATOM | 1939 | C | ILE | C3017 | 4.986 | −13.129 | 2.121 | 1.00 | 28.01 | C |
| ATOM | 1940 | O | ILE | C3017 | 5.160 | −14.133 | 1.426 | 1.00 | 27.90 | O |
| ATOM | 1941 | N | GLU | C3018 | 3.816 | −12.837 | 2.679 | 1.00 | 28.89 | N |
| ATOM | 1942 | CA | GLU | C3018 | 2.657 | −13.713 | 2.530 | 1.00 | 30.68 | C |
| ATOM | 1943 | CB | GLU | C3018 | 1.527 | −13.250 | 3.456 | 1.00 | 31.08 | C |
| ATOM | 1944 | CG | GLU | C3018 | 1.936 | −13.180 | 4.919 | 1.00 | 32.50 | C |
| ATOM | 1945 | CD | GLU | C3018 | 0.853 | −12.614 | 5.817 | 1.00 | 33.07 | C |
| ATOM | 1946 | OE1 | GLU | C3018 | 0.161 | −11.663 | 5.395 | 1.00 | 33.76 | O |
| ATOM | 1947 | OE2 | GLU | C3018 | 0.704 | −13.108 | 6.955 | 1.00 | 33.50 | O |
| ATOM | 1948 | C | GLU | C3018 | 2.144 | −13.796 | 1.092 | 1.00 | 31.38 | C |
| ATOM | 1949 | O | GLU | C3018 | 1.736 | −14.868 | 0.639 | 1.00 | 31.29 | O |
| ATOM | 1950 | N | SER | C3019 | 2.166 | −12.677 | 0.372 | 1.00 | 32.24 | N |
| ATOM | 1951 | CA | SER | C3019 | 1.691 | −12.676 | −1.008 | 1.00 | 33.36 | C |
| ATOM | 1952 | CB | SER | C3019 | 1.420 | −11.240 | −1.494 | 1.00 | 33.64 | C |
| ATOM | 1953 | OG | SER | C3019 | 2.610 | −10.547 | −1.838 | 1.00 | 34.47 | O |
| ATOM | 1954 | C | SER | C3019 | 2.706 | −13.364 | −1.918 | 1.00 | 34.14 | C |
| ATOM | 1955 | O | SER | C3019 | 2.331 | −14.055 | −2.864 | 1.00 | 34.07 | O |
| ATOM | 1956 | N | ALA | C3020 | 3.989 | −13.191 | −1.611 | 1.00 | 34.65 | N |
| ATOM | 1957 | CA | ALA | C3020 | 5.061 | −13.791 | −2.399 | 1.00 | 35.89 | C |
| ATOM | 1958 | CB | ALA | C3020 | 6.415 | −13.247 | −1.938 | 1.00 | 35.73 | C |
| ATOM | 1959 | C | ALA | C3020 | 5.071 | −15.322 | −2.347 | 1.00 | 36.89 | C |
| ATOM | 1960 | O | ALA | C3020 | 5.451 | −15.978 | −3.317 | 1.00 | 36.66 | O |
| ATOM | 1961 | N | VAL | C3021 | 4.663 | −15.891 | −1.217 | 1.00 | 38.20 | N |
| ATOM | 1962 | CA | VAL | C3021 | 4.640 | −17.343 | −1.078 | 1.00 | 40.04 | C |
| ATOM | 1963 | CB | VAL | C3021 | 4.270 | −17.770 | 0.362 | 1.00 | 40.00 | C |
| ATOM | 1964 | CG1 | VAL | C3021 | 4.180 | −19.285 | 0.445 | 1.00 | 40.22 | C |
| ATOM | 1965 | CG2 | VAL | C3021 | 5.310 | −17.255 | 1.347 | 1.00 | 40.17 | C |
| ATOM | 1966 | C | VAL | C3021 | 3.636 | −17.969 | −2.043 | 1.00 | 41.37 | C |
| ATOM | 1967 | O | VAL | C3021 | 3.939 | −18.954 | −2.718 | 1.00 | 41.44 | O |
| ATOM | 1968 | N | THR | C3022 | 2.443 | −17.387 | −2.105 | 1.00 | 42.72 | N |
| ATOM | 1969 | CA | THR | C3022 | 1.382 | −17.883 | −2.974 | 1.00 | 44.38 | C |
| ATOM | 1970 | CB | THR | C3022 | 0.049 | −17.160 | −2.683 | 1.00 | 43.84 | C |
| ATOM | 1971 | OG1 | THR | C3022 | −0.341 | −17.403 | −1.326 | 1.00 | 43.59 | O |
| ATOM | 1972 | CG2 | THR | C3022 | −1.048 | −17.665 | −3.612 | 1.00 | 43.81 | C |
| ATOM | 1973 | C | THR | C3022 | 1.703 | −17.738 | −4.461 | 1.00 | 45.84 | C |
| ATOM | 1974 | O | THR | C3022 | 1.725 | −18.730 | −5.193 | 1.00 | 45.92 | O |
| ATOM | 1975 | N | ARG | C3023 | 1.951 | −16.508 | −4.906 | 1.00 | 47.48 | N |
| ATOM | 1976 | CA | ARG | C3023 | 2.250 | −16.253 | −6.313 | 1.00 | 49.27 | C |
| ATOM | 1977 | CB | ARG | C3023 | 2.771 | −14.823 | −6.502 | 1.00 | 49.46 | C |
| ATOM | 1978 | CG | ARG | C3023 | 2.801 | −14.390 | −7.958 | 1.00 | 49.98 | C |
| ATOM | 1979 | CD | ARG | C3023 | 2.939 | −12.884 | −8.125 | 1.00 | 50.04 | C |
| ATOM | 1980 | NE | ARG | C3023 | 4.288 | −12.399 | −7.854 | 0.05 | 50.32 | N |
| ATOM | 1981 | CZ | ARG | C3023 | 4.694 | −11.158 | −8.105 | 0.05 | 50.40 | C |
| ATOM | 1982 | NH1 | ARG | C3023 | 3.853 | −10.279 | −8.634 | 0.05 | 50.48 | N |
| ATOM | 1983 | NH2 | ARG | C3023 | 5.940 | −10.797 | −7.833 | 0.05 | 50.49 | N |
| ATOM | 1984 | C | ARG | C3023 | 3.260 | −17.259 | −6.861 | 1.00 | 50.29 | C |
| ATOM | 1985 | O | ARG | C3023 | 2.889 | −18.186 | −7.580 | 1.00 | 50.77 | O |
| ATOM | 1986 | N | HIS | C3024 | 4.532 | −17.074 | −6.526 | 1.00 | 51.47 | N |
| ATOM | 1987 | CA | HIS | C3024 | 5.581 | −17.988 | −6.970 | 1.00 | 52.39 | C |
| ATOM | 1988 | CB | HIS | C3024 | 6.822 | −17.207 | −7.410 | 0.05 | 52.53 | C |
| ATOM | 1989 | CG | HIS | C3024 | 7.108 | −17.304 | −8.876 | 0.05 | 52.72 | C |
| ATOM | 1990 | CD2 | HIS | C3024 | 7.177 | −16.353 | −9.838 | 0.05 | 52.78 | C |
| ATOM | 1991 | ND1 | HIS | C3024 | 7.367 | −18.503 | −9.505 | 0.05 | 52.78 | N |
| ATOM | 1992 | CE1 | HIS | C3024 | 7.583 | −18.287 | −10.790 | 0.05 | 52.83 | C |
| ATOM | 1993 | NE2 | HIS | C3024 | 7.474 | −16.990 | −11.018 | 0.05 | 52.83 | N |
| ATOM | 1994 | C | HIS | C3024 | 5.931 | −18.911 | −5.806 | 1.00 | 53.02 | C |
| ATOM | 1995 | O | HIS | C3024 | 6.822 | −18.613 | −5.013 | 1.00 | 52.97 | O |
| ATOM | 1996 | N | GLU | C3025 | 5.222 | −20.032 | −5.712 | 1.00 | 53.71 | N |
| ATOM | 1997 | CA | GLU | C3025 | 5.430 | −20.992 | −4.632 | 1.00 | 54.23 | C |
| ATOM | 1998 | CB | GLU | C3025 | 4.308 | −22.035 | −4.647 | 1.00 | 55.24 | C |
| ATOM | 1999 | CG | GLU | C3025 | 4.150 | −22.772 | −5.965 | 1.00 | 56.63 | C |
| ATOM | 2000 | CD | GLU | C3025 | 2.869 | −23.585 | −6.024 | 1.00 | 57.48 | C |
| ATOM | 2001 | OE1 | GLU | C3025 | 2.657 | −24.435 | −5.131 | 1.00 | 58.10 | O |
| ATOM | 2002 | OE2 | GLU | C3025 | 2.075 | −23.372 | −6.966 | 1.00 | 57.87 | O |
| ATOM | 2003 | C | GLU | C3025 | 6.796 | −21.684 | −4.626 | 1.00 | 53.75 | C |
| ATOM | 2004 | O | GLU | C3025 | 6.916 | −22.841 | −4.227 | 1.00 | 53.95 | O |
| ATOM | 2005 | N | GLY | C3026 | 7.820 | −20.961 | −5.068 | 1.00 | 53.15 | N |
| ATOM | 2006 | CA | GLY | C3026 | 9.171 | −21.492 | −5.080 | 1.00 | 51.80 | C |
| ATOM | 2007 | C | GLY | C3026 | 10.057 | −20.503 | −4.340 | 1.00 | 50.97 | C |
| ATOM | 2008 | O | GLY | C3026 | 11.260 | −20.712 | −4.167 | 1.00 | 50.85 | O |
| ATOM | 2009 | N | PHE | C3027 | 9.426 | −19.418 | −3.899 | 1.00 | 49.86 | N |
| ATOM | 2010 | CA | PHE | C3027 | 10.077 | −18.331 | −3.173 | 1.00 | 48.78 | C |
| ATOM | 2011 | CB | PHE | C3027 | 9.001 | −17.419 | −2.570 | 1.00 | 48.81 | C |
| ATOM | 2012 | CG | PHE | C3027 | 9.546 | −16.198 | −1.884 | 1.00 | 48.66 | C |
| ATOM | 2013 | CD1 | PHE | C3027 | 10.279 | −15.252 | −2.593 | 1.00 | 48.61 | C |
| ATOM | 2014 | CD2 | PHE | C3027 | 9.313 | −15.986 | −0.527 | 1.00 | 48.69 | C |
| ATOM | 2015 | CE1 | PHE | C3027 | 10.772 | −14.111 | −1.962 | 1.00 | 48.58 | C |
| ATOM | 2016 | CE2 | PHE | C3027 | 9.802 | −14.849 | 0.113 | 1.00 | 48.35 | C |
| ATOM | 2017 | CZ | PHE | C3027 | 10.533 | −13.910 | −0.606 | 1.00 | 48.38 | C |
| ATOM | 2018 | C | PHE | C3027 | 11.037 | −18.795 | −2.076 | 1.00 | 47.88 | C |
| ATOM | 2019 | O | PHE | C3027 | 12.188 | −18.364 | −2.027 | 1.00 | 47.77 | O |
| ATOM | 2020 | N | ALA | C3028 | 10.558 | −19.676 | −1.205 | 1.00 | 47.03 | N |
| ATOM | 2021 | CA | ALA | C3028 | 11.362 | −20.186 | −0.097 | 1.00 | 46.36 | C |
| ATOM | 2022 | CB | ALA | C3028 | 10.529 | −21.147 | 0.741 | 1.00 | 46.30 | C |
| ATOM | 2023 | C | ALA | C3028 | 12.668 | −20.862 | −0.519 | 1.00 | 45.98 | C |
| ATOM | 2024 | O | ALA | C3028 | 13.735 | −20.562 | 0.026 | 1.00 | 46.02 | O |
| ATOM | 2025 | N | LYS | C3029 | 12.585 | −21.774 | −1.483 | 1.00 | 44.97 | N |
| ATOM | 2026 | CA | LYS | C3029 | 13.762 | −22.497 | −1.952 | 1.00 | 44.02 | C |
| ATOM | 2027 | CB | LYS | C3029 | 13.377 | −23.478 | −3.065 | 1.00 | 44.26 | C |
| ATOM | 2028 | CG | LYS | C3029 | 12.440 | −24.589 | −2.621 | 0.05 | 44.27 | C |
| ATOM | 2029 | CD | LYS | C3029 | 12.186 | −25.576 | −3.749 | 0.05 | 44.38 | C |
| ATOM | 2030 | CE | LYS | C3029 | 11.290 | −26.717 | −3.296 | 0.05 | 44.42 | C |
| ATOM | 2031 | NZ | LYS | C3029 | 11.066 | −27.712 | −4.380 | 0.05 | 44.48 | N |
| ATOM | 2032 | C | LYS | C3029 | 14.878 | −21.586 | −2.444 | 1.00 | 43.30 | C |
| ATOM | 2033 | O | LYS | C3029 | 16.055 | −21.926 | −2.330 | 1.00 | 43.66 | O |
| ATOM | 2034 | N | ARG | C3030 | 14.516 | −20.429 | −2.988 | 1.00 | 42.33 | N |
| ATOM | 2035 | CA | ARG | C3030 | 15.515 | −19.498 | −3.495 | 1.00 | 41.38 | C |
| ATOM | 2036 | CB | ARG | C3030 | 14.933 | −18.685 | −4.655 | 1.00 | 41.73 | C |
| ATOM | 2037 | CG | ARG | C3030 | 14.577 | −19.527 | −5.870 | 0.05 | 41.77 | C |
| ATOM | 2038 | CD | ARG | C3030 | 14.062 | −18.672 | −7.016 | 0.05 | 41.90 | C |
| ATOM | 2039 | NE | ARG | C3030 | 13.773 | −19.474 | −8.202 | 0.05 | 42.00 | N |
| ATOM | 2040 | CZ | ARG | C3030 | 13.329 | −18.977 | −9.351 | 0.05 | 42.04 | C |
| ATOM | 2041 | NH1 | ARG | C3030 | 13.092 | −19.784 | −10.377 | 0.05 | 42.08 | N |
| ATOM | 2042 | NH2 | ARG | C3030 | 13.119 | −17.674 | −9.476 | 0.05 | 42.08 | N |
| ATOM | 2043 | C | ARG | C3030 | 16.073 | −18.551 | −2.435 | 1.00 | 40.66 | C |
| ATOM | 2044 | O | ARG | C3030 | 17.141 | −17.969 | −2.620 | 1.00 | 40.56 | O |
| ATOM | 2045 | N | VAL | C3031 | 15.357 | −18.402 | −1.324 | 1.00 | 39.61 | N |
| ATOM | 2046 | CA | VAL | C3031 | 15.797 | −17.509 | −0.252 | 1.00 | 38.51 | C |
| ATOM | 2047 | CB | VAL | C3031 | 14.593 | −16.737 | 0.354 | 1.00 | 38.68 | C |
| ATOM | 2048 | CG1 | VAL | C3031 | 15.046 | −15.899 | 1.545 | 1.00 | 39.01 | C |
| ATOM | 2049 | CG2 | VAL | C3031 | 13.964 | −15.843 | −0.703 | 1.00 | 39.32 | C |
| ATOM | 2050 | C | VAL | C3031 | 16.535 | −18.222 | 0.887 | 1.00 | 37.22 | C |
| ATOM | 2051 | O | VAL | C3031 | 17.392 | −17.625 | 1.540 | 1.00 | 36.96 | O |
| ATOM | 2052 | N | LEU | C3032 | 16.215 | −19.494 | 1.109 | 1.00 | 35.97 | N |
| ATOM | 2053 | CA | LEU | C3032 | 16.825 | −20.268 | 2.194 | 1.00 | 35.20 | C |
| ATOM | 2054 | CB | LEU | C3032 | 15.713 | −20.865 | 3.062 | 1.00 | 34.43 | C |
| ATOM | 2055 | CG | LEU | C3032 | 14.682 | −19.887 | 3.635 | 1.00 | 34.07 | C |
| ATOM | 2056 | CD1 | LEU | C3032 | 13.508 | −20.658 | 4.224 | 1.00 | 34.12 | C |
| ATOM | 2057 | CD2 | LEU | C3032 | 15.340 | −19.010 | 4.694 | 1.00 | 33.54 | C |
| ATOM | 2058 | C | LEU | C3032 | 17.774 | −21.395 | 1.756 | 1.00 | 35.04 | C |
| ATOM | 2059 | O | LEU | C3032 | 17.592 | −21.997 | 0.696 | 1.00 | 34.60 | O |
| ATOM | 2060 | N | THR | C3033 | 18.780 | −21.679 | 2.584 | 1.00 | 34.45 | N |
| ATOM | 2061 | CA | THR | C3033 | 19.729 | −22.755 | 2.293 | 1.00 | 34.22 | C |
| ATOM | 2062 | CB | THR | C3033 | 21.050 | −22.619 | 3.096 | 1.00 | 33.96 | C |
| ATOM | 2063 | OG1 | THR | C3033 | 20.797 | −22.889 | 4.481 | 1.00 | 33.02 | O |
| ATOM | 2064 | CG2 | THR | C3033 | 21.630 | −21.222 | 2.953 | 1.00 | 34.21 | C |
| ATOM | 2065 | C | THR | C3033 | 19.063 | −24.063 | 2.720 | 1.00 | 34.16 | C |
| ATOM | 2066 | O | THR | C3033 | 17.968 | −24.052 | 3.279 | 1.00 | 33.78 | O |
| ATOM | 2067 | N | ALA | C3034 | 19.729 | −25.185 | 2.472 | 1.00 | 34.40 | N |
| ATOM | 2068 | CA | ALA | C3034 | 19.178 | −26.487 | 2.839 | 1.00 | 34.48 | C |
| ATOM | 2069 | CB | ALA | C3034 | 20.103 | −27.598 | 2.350 | 1.00 | 34.65 | C |
| ATOM | 2070 | C | ALA | C3034 | 18.927 | −26.638 | 4.341 | 1.00 | 34.51 | C |
| ATOM | 2071 | O | ALA | C3034 | 17.867 | −27.117 | 4.751 | 1.00 | 34.56 | O |
| ATOM | 2072 | N | LEU | C3035 | 19.895 | −26.233 | 5.162 | 1.00 | 34.52 | N |
| ATOM | 2073 | CA | LEU | C3035 | 19.755 | −26.345 | 6.612 | 1.00 | 34.61 | C |
| ATOM | 2074 | CB | LEU | C3035 | 21.087 | −26.047 | 7.308 | 1.00 | 34.52 | C |
| ATOM | 2075 | CG | LEU | C3035 | 21.101 | −26.182 | 8.834 | 0.05 | 34.52 | C |
| ATOM | 2076 | CD1 | LEU | C3035 | 20.741 | −27.607 | 9.228 | 0.05 | 34.51 | C |
| ATOM | 2077 | CD2 | LEU | C3035 | 22.475 | −25.814 | 9.369 | 0.05 | 34.53 | C |
| ATOM | 2078 | C | LEU | C3035 | 18.675 | −25.414 | 7.156 | 1.00 | 35.00 | C |
| ATOM | 2079 | O | LEU | C3035 | 17.965 | −25.769 | 8.098 | 1.00 | 35.04 | O |
| ATOM | 2080 | N | GLU | C3036 | 18.558 | −24.222 | 6.574 | 1.00 | 35.33 | N |
| ATOM | 2081 | CA | GLU | C3036 | 17.537 | −23.263 | 7.004 | 1.00 | 35.62 | C |
| ATOM | 2082 | CB | GLU | C3036 | 17.741 | −21.907 | 6.310 | 1.00 | 34.70 | C |
| ATOM | 2083 | CG | GLU | C3036 | 18.809 | −21.006 | 6.941 | 1.00 | 33.21 | C |
| ATOM | 2084 | CD | GLU | C3036 | 19.117 | −19.767 | 6.103 | 1.00 | 32.82 | C |
| ATOM | 2085 | OE1 | GLU | C3036 | 19.598 | −18.759 | 6.671 | 1.00 | 31.24 | O |
| ATOM | 2086 | OE2 | GLU | C3036 | 18.893 | −19.803 | 4.873 | 1.00 | 32.21 | O |
| ATOM | 2087 | C | GLU | C3036 | 16.155 | −23.815 | 6.654 | 1.00 | 36.75 | C |
| ATOM | 2088 | O | GLU | C3036 | 15.205 | −23.687 | 7.427 | 1.00 | 36.40 | O |
| ATOM | 2089 | N | MET | C3037 | 16.064 | −24.436 | 5.481 | 1.00 | 38.24 | N |
| ATOM | 2090 | CA | MET | C3037 | 14.821 | −25.018 | 4.981 | 1.00 | 40.09 | C |
| ATOM | 2091 | CB | MET | C3037 | 15.064 | −25.625 | 3.596 | 1.00 | 41.14 | C |
| ATOM | 2092 | CG | MET | C3037 | 13.808 | −26.028 | 2.834 | 1.00 | 43.15 | C |
| ATOM | 2093 | SD | MET | C3037 | 12.851 | −24.618 | 2.239 | 1.00 | 45.06 | S |
| ATOM | 2094 | CE | MET | C3037 | 11.189 | −25.242 | 2.400 | 1.00 | 44.92 | C |
| ATOM | 2095 | C | MET | C3037 | 14.270 | −26.089 | 5.926 | 1.00 | 40.63 | C |
| ATOM | 2096 | O | MET | C3037 | 13.061 | −26.171 | 6.151 | 1.00 | 40.94 | O |
| ATOM | 2097 | N | GLU | C3038 | 15.159 | −26.911 | 6.473 | 1.00 | 41.23 | N |
| ATOM | 2098 | CA | GLU | C3038 | 14.752 | −27.969 | 7.393 | 1.00 | 41.49 | C |
| ATOM | 2099 | CB | GLU | C3038 | 15.982 | −28.713 | 7.924 | 1.00 | 41.61 | C |
| ATOM | 2100 | CG | GLU | C3038 | 16.711 | −29.544 | 6.878 | 0.05 | 41.50 | C |
| ATOM | 2101 | CD | GLU | C3038 | 15.889 | −30.721 | 6.385 | 0.05 | 41.51 | C |
| ATOM | 2102 | OE1 | GLU | C3038 | 16.376 | −31.459 | 5.503 | 0.05 | 41.48 | O |
| ATOM | 2103 | OE2 | GLU | C3038 | 14.758 | −30.911 | 6.880 | 0.05 | 41.45 | O |
| ATOM | 2104 | C | GLU | C3038 | 13.958 | −27.391 | 8.559 | 1.00 | 41.78 | C |
| ATOM | 2105 | O | GLU | C3038 | 12.984 | −27.989 | 9.013 | 1.00 | 41.81 | O |
| ATOM | 2106 | N | ARG | C3039 | 14.384 | −26.226 | 9.037 | 1.00 | 42.11 | N |
| ATOM | 2107 | CA | ARG | C3039 | 13.716 | −25.550 | 10.146 | 1.00 | 42.69 | C |
| ATOM | 2108 | CB | ARG | C3039 | 14.580 | −24.380 | 10.634 | 1.00 | 43.67 | C |
| ATOM | 2109 | CG | ARG | C3039 | 13.950 | −23.514 | 11.722 | 1.00 | 45.06 | C |
| ATOM | 2110 | CD | ARG | C3039 | 13.752 | −24.276 | 13.026 | 1.00 | 46.69 | C |
| ATOM | 2111 | NE | ARG | C3039 | 13.295 | −23.394 | 14.099 | 1.00 | 47.90 | N |
| ATOM | 2112 | CZ | ARG | C3039 | 12.966 | −23.803 | 15.321 | 1.00 | 48.72 | C |
| ATOM | 2113 | NH1 | ARG | C3039 | 13.040 | −25.089 | 15.637 | 1.00 | 49.17 | N |
| ATOM | 2114 | NH2 | ARG | C3039 | 12.563 | −22.925 | 16.230 | 1.00 | 49.02 | N |
| ATOM | 2115 | C | ARG | C3039 | 12.343 | −25.034 | 9.714 | 1.00 | 42.44 | C |
| ATOM | 2116 | O | ARG | C3039 | 11.360 | −25.153 | 10.448 | 1.00 | 42.21 | O |
| ATOM | 2117 | N | PHE | C3040 | 12.293 | −24.467 | 8.512 | 1.00 | 42.44 | N |
| ATOM | 2118 | CA | PHE | C3040 | 11.064 | −23.909 | 7.948 | 1.00 | 42.58 | C |
| ATOM | 2119 | CB | PHE | C3040 | 11.343 | −23.394 | 6.529 | 1.00 | 41.84 | C |
| ATOM | 2120 | CG | PHE | C3040 | 10.165 | −22.713 | 5.879 | 1.00 | 41.26 | C |
| ATOM | 2121 | CD1 | PHE | C3040 | 9.621 | −21.556 | 6.428 | 1.00 | 40.76 | C |
| ATOM | 2122 | CD2 | PHE | C3040 | 9.610 | −23.223 | 4.708 | 1.00 | 40.96 | C |
| ATOM | 2123 | CE1 | PHE | C3040 | 8.541 | −20.915 | 5.821 | 1.00 | 40.74 | C |
| ATOM | 2124 | CE2 | PHE | C3040 | 8.527 | −22.590 | 4.090 | 1.00 | 40.92 | C |
| ATOM | 2125 | CZ | PHE | C3040 | 7.992 | −21.433 | 4.649 | 1.00 | 40.88 | C |
| ATOM | 2126 | C | PHE | C3040 | 9.899 | −24.902 | 7.913 | 1.00 | 43.12 | C |
| ATOM | 2127 | O | PHE | C3040 | 8.790 | −24.585 | 8.343 | 1.00 | 42.92 | O |
| ATOM | 2128 | N | THR | C3041 | 10.156 | −26.104 | 7.406 | 1.00 | 43.77 | N |
| ATOM | 2129 | CA | THR | C3041 | 9.119 | −27.126 | 7.303 | 1.00 | 44.43 | C |
| ATOM | 2130 | CB | THR | C3041 | 9.569 | −22.283 | 6.395 | 1.00 | 44.80 | C |
| ATOM | 2131 | OG1 | THR | C3041 | 10.703 | −28.936 | 6.978 | 1.00 | 45.05 | O |
| ATOM | 2132 | CG2 | THR | C3041 | 9.935 | −27.759 | 5.016 | 1.00 | 45.00 | C |
| ATOM | 2133 | C | THR | C3041 | 8.676 | −27.719 | 8.635 | 1.00 | 44.57 | C |
| ATOM | 2134 | O | THR | C3041 | 7.611 | −28.327 | 8.721 | 1.00 | 44.92 | O |
| ATOM | 2135 | N | SER | C3042 | 9.488 | −27.548 | 9.672 | 1.00 | 44.91 | N |
| ATOM | 2136 | CA | SER | C3042 | 9.160 | −28.091 | 10.986 | 1.00 | 44.98 | C |
| ATOM | 2137 | CB | SER | C3042 | 10.439 | −28.297 | 11.801 | 1.00 | 45.18 | C |
| ATOM | 2138 | OG | SER | C3042 | 11.336 | −29.151 | 11.110 | 1.00 | 45.91 | O |
| ATOM | 2139 | C | SER | C3042 | 8.194 | −27.212 | 11.770 | 1.00 | 44.99 | C |
| ATOM | 2140 | O | SER | C3042 | 7.536 | −27.683 | 12.699 | 1.00 | 45.11 | O |
| ATOM | 2141 | N | LEU | C3043 | 8.112 | −25.938 | 11.397 | 1.00 | 44.68 | N |
| ATOM | 2142 | CA | LEU | C3043 | 7.228 | −24.993 | 12.073 | 1.00 | 44.47 | C |
| ATOM | 2143 | CB | LEU | C3043 | 7.882 | −23.608 | 12.124 | 1.00 | 44.33 | C |
| ATOM | 2144 | CG | LEU | C3043 | 9.250 | −23.485 | 12.797 | 1.00 | 44.21 | C |
| ATOM | 2145 | CD1 | LEU | C3043 | 9.761 | −22.052 | 12.662 | 1.00 | 44.22 | C |
| ATOM | 2146 | CD2 | LEU | C3043 | 9.139 | −23.875 | 14.265 | 1.00 | 44.47 | C |
| ATOM | 2147 | C | LEU | C3043 | 5.888 | −24.899 | 11.347 | 1.00 | 44.31 | C |
| ATOM | 2148 | O | LEU | C3043 | 5.738 | −25.422 | 10.240 | 1.00 | 44.09 | O |
| ATOM | 2149 | N | LYS | C3044 | 4.923 | −24.221 | 11.965 | 1.00 | 44.27 | N |
| ATOM | 2150 | CA | LYS | C3044 | 3.601 | −24.064 | 11.365 | 1.00 | 44.15 | C |
| ATOM | 2151 | CB | LYS | C3044 | 2.703 | −25.234 | 11.777 | 1.00 | 45.11 | C |
| ATOM | 2152 | CG | LYS | C3044 | 1.429 | −25.375 | 10.953 | 1.00 | 46.10 | C |
| ATOM | 2153 | CD | LYS | C3044 | 0.462 | −26.398 | 11.547 | 1.00 | 47.10 | C |
| ATOM | 2154 | CE | LYS | C3044 | 1.099 | −27.774 | 11.701 | 1.00 | 47.88 | C |
| ATOM | 2155 | NZ | LYS | C3044 | 2.155 | −27.802 | 12.758 | 1.00 | 48.36 | N |
| ATOM | 2156 | C | LYS | C3044 | 2.924 | −22.745 | 11.753 | 1.00 | 43.58 | C |
| ATOM | 2157 | O | LYS | C3044 | 3.349 | −22.062 | 12.687 | 1.00 | 43.58 | O |
| ATOM | 2158 | N | GLY | C3045 | 1.868 | −22.397 | 11.022 | 1.00 | 42.72 | N |
| ATOM | 2159 | CA | GLY | C3045 | 1.129 | −21.177 | 11.299 | 1.00 | 41.65 | C |
| ATOM | 2160 | C | GLY | C3045 | 1.932 | −19.893 | 11.197 | 1.00 | 40.64 | C |
| ATOM | 2161 | O | GLY | C3045 | 2.788 | −19.749 | 10.323 | 1.00 | 40.57 | O |
| ATOM | 2162 | N | ARG | C3046 | 1.644 | −18.956 | 12.097 | 1.00 | 39.78 | N |
| ATOM | 2163 | CA | ARG | C3046 | 2.327 | −17.666 | 12.129 | 1.00 | 38.64 | C |
| ATOM | 2164 | CB | ARG | C3046 | 1.752 | −16.792 | 13.249 | 1.00 | 38.93 | C |
| ATOM | 2165 | CG | ARG | C3046 | 2.393 | −15.415 | 13.350 | 0.05 | 38.89 | C |
| ATOM | 2166 | CD | ARG | C3046 | 1.769 | −14.591 | 14.465 | 0.05 | 38.99 | C |
| ATOM | 2167 | NE | ARG | C3046 | 0.346 | −14.352 | 14.241 | 0.05 | 39.04 | N |
| ATOM | 2168 | CZ | ARG | C3046 | −0.428 | −13.636 | 15.050 | 0.05 | 39.08 | C |
| ATOM | 2169 | NH1 | ARG | C3046 | −1.713 | −13.471 | 14.768 | 0.05 | 39.09 | N |
| ATOM | 2170 | NH2 | ARG | C3046 | 0.084 | −13.084 | 16.142 | 0.05 | 39.12 | N |
| ATOM | 2171 | C | ARG | C3046 | 3.826 | −17.840 | 12.338 | 1.00 | 37.81 | C |
| ATOM | 2172 | O | ARG | C3046 | 4.629 | −17.130 | 11.735 | 1.00 | 37.34 | O |
| ATOM | 2173 | N | ARG | C3047 | 4.195 | −18.785 | 13.198 | 1.00 | 36.94 | N |
| ATOM | 2174 | CA | ARG | C3047 | 5.601 | −19.056 | 13.485 | 1.00 | 35.80 | C |
| ATOM | 2175 | CB | ARG | C3047 | 5.727 | −20.255 | 14.424 | 1.00 | 37.34 | C |
| ATOM | 2176 | CG | ARG | C3047 | 5.554 | −19.924 | 15.887 | 1.00 | 38.72 | C |
| ATOM | 2177 | CD | ARG | C3047 | 5.695 | −21.177 | 16.731 | 1.00 | 40.55 | C |
| ATOM | 2178 | NE | ARG | C3047 | 6.145 | −20.870 | 18.083 | 1.00 | 41.86 | N |
| ATOM | 2179 | CZ | ARG | C3047 | 7.328 | −20.335 | 18.369 | 1.00 | 42.33 | C |
| ATOM | 2180 | NH1 | ARG | C3047 | 8.181 | −20.050 | 17.395 | 1.00 | 43.10 | N |
| ATOM | 2181 | NH2 | ARG | C3047 | 7.659 | −20.087 | 19.629 | 1.00 | 42.88 | N |
| ATOM | 2182 | C | ARG | C3047 | 6.408 | −19.329 | 12.223 | 1.00 | 34.36 | C |
| ATOM | 2183 | O | ARG | C3047 | 7.518 | −18.812 | 12.057 | 1.00 | 33.67 | O |
| ATOM | 2184 | N | GLN | C3048 | 5.852 | −20.152 | 11.340 | 1.00 | 32.32 | N |
| ATOM | 2185 | CA | GLN | C3048 | 6.522 | −20.493 | 10.095 | 1.00 | 30.19 | C |
| ATOM | 2186 | CB | GLN | C3048 | 5.709 | −21.538 | 9.326 | 1.00 | 30.56 | C |
| ATOM | 2187 | CG | GLN | C3048 | 6.346 | −21.947 | 8.010 | 1.00 | 31.08 | C |
| ATOM | 2188 | CD | GLN | C3048 | 5.545 | −22.989 | 7.256 | 1.00 | 31.55 | C |
| ATOM | 2189 | OE1 | GLN | C3048 | 4.363 | −22.793 | 6.971 | 1.00 | 32.61 | O |
| ATOM | 2190 | NE2 | GLN | C3048 | 6.189 | −24.100 | 6.919 | 1.00 | 31.23 | N |
| ATOM | 2191 | C | GLN | C3048 | 6.749 | −19.266 | 9.211 | 1.00 | 28.78 | C |
| ATOM | 2192 | O | GLN | C3048 | 7.816 | −19.113 | 8.615 | 1.00 | 27.77 | O |
| ATOM | 2193 | N | ILE | C3049 | 5.747 | −18.396 | 9.124 | 1.00 | 27.16 | N |
| ATOM | 2194 | CA | ILE | C3049 | 5.862 | −17.195 | 8.298 | 1.00 | 26.51 | C |
| ATOM | 2195 | CB | ILE | C3049 | 4.485 | −16.521 | 8.086 | 1.00 | 26.30 | C |
| ATOM | 2196 | CG2 | ILE | C3049 | 4.647 | −15.250 | 7.255 | 1.00 | 26.17 | C |
| ATOM | 2197 | CG1 | ILE | C3049 | 3.530 | −17.491 | 7.380 | 1.00 | 26.22 | C |
| ATOM | 2198 | CD1 | ILE | C3049 | 4.023 | −17.978 | 6.014 | 1.00 | 26.61 | C |
| ATOM | 2199 | C | ILE | C3049 | 6.831 | −16.178 | 8.910 | 1.00 | 25.94 | C |
| ATOM | 2200 | O | ILE | C3049 | 7.570 | −15.510 | 8.192 | 1.00 | 25.27 | O |
| ATOM | 2201 | N | GLU | C3050 | 6.824 | −16.069 | 10.236 | 1.00 | 26.02 | N |
| ATOM | 2202 | CA | GLU | C3050 | 7.716 | −15.141 | 10.935 | 1.00 | 25.96 | C |
| ATOM | 2203 | CB | GLU | C3050 | 7.347 | −15.075 | 12.423 | 1.00 | 27.39 | C |
| ATOM | 2204 | CG | GLU | C3050 | 6.073 | −14.287 | 12.724 | 1.00 | 29.92 | C |
| ATOM | 2205 | CD | GLU | C3050 | 6.345 | −12.853 | 13.175 | 1.00 | 31.52 | C |
| ATOM | 2206 | OE1 | GLU | C3050 | 7.446 | −12.322 | 12.894 | 1.00 | 32.57 | O |
| ATOM | 2207 | OE2 | GLU | C3050 | 5.444 | −12.252 | 13.802 | 1.00 | 31.86 | O |
| ATOM | 2208 | C | GLU | C3050 | 9.195 | −15.521 | 10.792 | 1.00 | 24.88 | C |
| ATOM | 2209 | O | GLU | C3050 | 10.061 | −14.650 | 10.815 | 1.00 | 24.17 | O |
| ATOM | 2210 | N | TYR | C3051 | 9.481 | −16.815 | 10.652 | 1.00 | 23.54 | N |
| ATOM | 2211 | CA | TYR | C3051 | 10.863 | −17.281 | 10.508 | 1.00 | 22.59 | C |
| ATOM | 2212 | CB | TYR | C3051 | 10.941 | −18.808 | 10.711 | 1.00 | 23.12 | C |
| ATOM | 2213 | CG | TYR | C3051 | 12.317 | −19.425 | 10.473 | 1.00 | 23.17 | C |
| ATOM | 2214 | CD1 | TYR | C3051 | 12.685 | −19.902 | 9.213 | 1.00 | 23.85 | C |
| ATOM | 2215 | CE1 | TYR | C3051 | 13.954 | −20.454 | 8.984 | 1.00 | 24.32 | C |
| ATOM | 2216 | CD2 | TYR | C3051 | 13.253 | −19.515 | 11.504 | 1.00 | 24.30 | C |
| ATOM | 2217 | CE2 | TYR | C3051 | 14.528 | −20.064 | 11.288 | 1.00 | 24.54 | C |
| ATOM | 2218 | CZ | TYR | C3051 | 14.868 | −20.528 | 10.026 | 1.00 | 24.99 | C |
| ATOM | 2219 | OH | TYR | C3051 | 16.122 | −21.050 | 9.803 | 1.00 | 25.78 | O |
| ATOM | 2220 | C | TYR | C3051 | 11.419 | −16.894 | 9.140 | 1.00 | 22.18 | C |
| ATOM | 2221 | O | TYR | C3051 | 12.546 | −16.395 | 9.031 | 1.00 | 20.97 | O |
| ATOM | 2222 | N | LEU | C3052 | 10.618 | −17.115 | 8.101 | 1.00 | 21.39 | N |
| ATOM | 2223 | CA | LEU | C3052 | 11.016 | −16.787 | 6.736 | 1.00 | 21.55 | C |
| ATOM | 2224 | CB | LEU | C3052 | 9.935 | −17.254 | 5.747 | 1.00 | 22.13 | C |
| ATOM | 2225 | CG | LEU | C3052 | 10.124 | −16.929 | 4.259 | 1.00 | 21.93 | C |
| ATOM | 2226 | CD1 | LEU | C3052 | 11.439 | −17.515 | 3.755 | 1.00 | 22.55 | C |
| ATOM | 2227 | CD2 | LEU | C3052 | 8.958 | −17.495 | 3.469 | 1.00 | 22.75 | C |
| ATOM | 2228 | C | LEU | C3052 | 11.245 | −15.284 | 6.574 | 1.00 | 21.31 | C |
| ATOM | 2229 | O | LEU | C3052 | 12.196 | −14.866 | 5.914 | 1.00 | 21.85 | O |
| ATOM | 2230 | N | ALA | C3053 | 10.371 | −14.484 | 7.181 | 1.00 | 20.68 | N |
| ATOM | 2231 | CA | ALA | C3053 | 10.454 | −13.025 | 7.102 | 1.00 | 20.75 | C |
| ATOM | 2232 | CB | ALA | C3053 | 9.203 | −12.401 | 7.738 | 1.00 | 20.99 | C |
| ATOM | 2233 | C | ALA | C3053 | 11.720 | −12.469 | 7.768 | 1.00 | 20.60 | C |
| ATOM | 2234 | O | ALA | C3053 | 12.356 | −11.552 | 7.237 | 1.00 | 20.36 | O |
| ATOM | 2235 | N | GLY | C3054 | 12.073 | −13.027 | 8.925 | 1.00 | 20.45 | N |
| ATOM | 2236 | CA | GLY | C3054 | 13.264 | −12.597 | 9.646 | 1.00 | 20.63 | C |
| ATOM | 2237 | C | GLY | C3054 | 14.576 | −12.928 | 8.945 | 1.00 | 20.53 | C |
| ATOM | 2238 | O | GLY | C3054 | 15.550 | −12.174 | 9.034 | 1.00 | 19.61 | O |
| ATOM | 2239 | N | ARG | C3055 | 14.620 | −14.065 | 8.256 | 1.00 | 20.88 | N |
| ATOM | 2240 | CA | ARG | C3055 | 15.824 | −14.457 | 7.527 | 1.00 | 21.09 | C |
| ATOM | 2241 | CB | ARG | C3055 | 15.731 | −15.930 | 7.101 | 1.00 | 21.81 | C |
| ATOM | 2242 | CG | ARG | C3055 | 16.533 | −16.895 | 7.988 | 1.00 | 23.24 | C |
| ATOM | 2243 | CD | ARG | C3055 | 16.139 | −16.801 | 9.465 | 1.00 | 23.82 | C |
| ATOM | 2244 | NE | ARG | C3055 | 17.018 | −17.600 | 10.325 | 1.00 | 23.91 | N |
| ATOM | 2245 | CZ | ARG | C3055 | 16.881 | −17.706 | 11.646 | 1.00 | 24.64 | C |
| ATOM | 2246 | NH1 | ARG | C3055 | 15.901 | −17.065 | 12.267 | 1.00 | 23.93 | N |
| ATOM | 2247 | NH2 | ARG | C3055 | 17.719 | −18.459 | 12.352 | 1.00 | 25.21 | N |
| ATOM | 2248 | C | ARG | C3055 | 16.017 | −13.552 | 6.301 | 1.00 | 21.23 | C |
| ATOM | 2249 | O | ARG | C3055 | 17.142 | −13.234 | 5.921 | 1.00 | 20.84 | O |
| ATOM | 2250 | N | TRP | C3056 | 14.911 | −13.132 | 5.694 | 1.00 | 21.62 | N |
| ATOM | 2251 | CA | TRP | C3056 | 14.958 | −12.247 | 4.529 | 1.00 | 22.92 | C |
| ATOM | 2252 | CB | TRP | C3056 | 13.559 | −12.121 | 3.910 | 1.00 | 25.18 | C |
| ATOM | 2253 | CG | TRP | C3056 | 13.515 | −11.388 | 2.594 | 1.00 | 27.65 | C |
| ATOM | 2254 | CD2 | TRP | C3056 | 13.357 | −9.977 | 2.401 | 1.00 | 28.51 | C |
| ATOM | 2255 | CE2 | TRP | C3056 | 13.413 | −9.736 | 1.009 | 1.00 | 29.11 | C |
| ATOM | 2256 | CE3 | TRP | C3056 | 13.176 | −8.892 | 3.269 | 1.00 | 29.11 | C |
| ATOM | 2257 | CD1 | TRP | C3056 | 13.656 | −11.931 | 1.344 | 1.00 | 28.78 | C |
| ATOM | 2258 | NE1 | TRP | C3056 | 13.596 | −10.944 | 0.388 | 1.00 | 29.88 | N |
| ATOM | 2259 | CZ2 | TRP | C3056 | 13.296 | −8.453 | 0.465 | 1.00 | 28.95 | C |
| ATOM | 2260 | CZ3 | TRP | C3056 | 13.060 | −7.614 | 2.726 | 1.00 | 29.68 | C |
| ATOM | 2261 | CH2 | TRP | C3056 | 13.121 | −7.409 | 1.336 | 1.00 | 29.31 | C |
| ATOM | 2262 | C | TRP | C3056 | 15.453 | −10.855 | 4.953 | 1.00 | 22.17 | C |
| ATOM | 2263 | O | TRP | C3056 | 16.286 | −10.247 | 4.281 | 1.00 | 22.33 | O |
| ATOM | 2264 | N | SER | C3057 | 14.942 | −10.365 | 6.078 | 1.00 | 21.01 | N |
| ATOM | 2265 | CA | SER | C3057 | 15.311 | −9.041 | 6.587 | 1.00 | 20.66 | C |
| ATOM | 2266 | CB | SER | C3057 | 14.388 | −8.663 | 7.751 | 1.00 | 21.23 | C |
| ATOM | 2267 | OG | SER | C3057 | 14.569 | −7.311 | 8.147 | 1.00 | 22.78 | O |
| ATOM | 2268 | C | SER | C3057 | 16.786 | −8.969 | 7.025 | 1.00 | 20.18 | C |
| ATOM | 2269 | O | SER | C3057 | 17.464 | −7.959 | 6.803 | 1.00 | 19.84 | O |
| ATOM | 2270 | N | ALA | C3058 | 17.280 | −10.043 | 7.633 | 1.00 | 18.66 | N |
| ATOM | 2271 | CA | ALA | C3058 | 18.671 | −10.090 | 8.085 | 1.00 | 18.83 | C |
| ATOM | 2272 | CB | ALA | C3058 | 18.905 | −11.344 | 8.939 | 1.00 | 17.30 | C |
| ATOM | 2273 | C | ALA | C3058 | 19.663 | −10.062 | 6.915 | 1.00 | 18.44 | C |
| ATOM | 2274 | O | ALA | C3058 | 20.693 | −9.381 | 6.980 | 1.00 | 18.07 | O |
| ATOM | 2275 | N | LYS | C3059 | 19.359 | −10.803 | 5.851 | 1.00 | 18.60 | N |
| ATOM | 2276 | CA | LYS | C3059 | 20.252 | −10.850 | 4.692 | 1.00 | 18.76 | C |
| ATOM | 2277 | CB | LYS | C3059 | 19.848 | −12.012 | 3.766 | 1.00 | 18.60 | C |
| ATOM | 2278 | CG | LYS | C3059 | 19.956 | −13.396 | 4.447 | 1.00 | 18.86 | C |
| ATOM | 2279 | CD | LYS | C3059 | 19.739 | −14.578 | 3.494 | 1.00 | 19.28 | C |
| ATOM | 2280 | CE | LYS | C3059 | 19.815 | −15.920 | 4.244 | 1.00 | 20.00 | C |
| ATOM | 2281 | NZ | LYS | C3059 | 19.590 | −17.130 | 3.388 | 1.00 | 20.93 | N |
| ATOM | 2282 | C | LYS | C3059 | 20.274 | −9.508 | 3.938 | 1.00 | 19.50 | C |
| ATOM | 2283 | O | LYS | C3059 | 21.306 | −9.117 | 3.388 | 1.00 | 20.13 | O |
| ATOM | 2284 | N | GLU | C3060 | 19.142 | −8.804 | 3.935 | 1.00 | 20.20 | N |
| ATOM | 2285 | CA | GLU | C3060 | 19.026 | −7.501 | 3.277 | 1.00 | 20.95 | C |
| ATOM | 2286 | CB | GLU | C3060 | 17.550 | −7.090 | 3.205 | 1.00 | 23.10 | C |
| ATOM | 2287 | CG | GLU | C3060 | 17.268 | −5.738 | 2.545 | 1.00 | 26.69 | C |
| ATOM | 2288 | CD | GLU | C3060 | 17.579 | −5.717 | 1.055 | 1.00 | 29.57 | C |
| ATOM | 2289 | OE1 | GLU | C3060 | 17.161 | −6.660 | 0.341 | 1.00 | 30.70 | O |
| ATOM | 2290 | OE2 | GLU | C3060 | 18.232 | −4.749 | 0.598 | 1.00 | 31.10 | O |
| ATOM | 2291 | C | GLU | C3060 | 19.837 | −6.432 | 4.030 | 1.00 | 20.53 | C |
| ATOM | 2292 | O | GLU | C3060 | 20.455 | −5.563 | 3.414 | 1.00 | 20.28 | O |
| ATOM | 2293 | N | ALA | C3061 | 19.838 | −6.497 | 5.360 | 1.00 | 19.74 | N |
| ATOM | 2294 | CA | ALA | C3061 | 20.591 | −5.531 | 6.165 | 1.00 | 19.24 | C |
| ATOM | 2295 | CB | ALA | C3061 | 20.252 | −5.696 | 7.656 | 1.00 | 19.62 | C |
| ATOM | 2296 | C | ALA | C3061 | 22.097 | −5.711 | 5.938 | 1.00 | 18.92 | C |
| ATOM | 2297 | O | ALA | C3061 | 22.848 | −4.732 | 5.885 | 1.00 | 18.37 | O |
| ATOM | 2298 | N | PHE | C3062 | 22.530 | −6.963 | 5.811 | 1.00 | 18.75 | N |
| ATOM | 2299 | CA | PHE | C3062 | 23.941 | −7.270 | 5.570 | 1.00 | 19.65 | C |
| ATOM | 2300 | CB | PHE | C3062 | 24.190 | −8.783 | 5.714 | 1.00 | 19.70 | C |
| ATOM | 2301 | CG | PHE | C3062 | 25.592 | −9.224 | 5.325 | 1.00 | 20.12 | C |
| ATOM | 2302 | CD1 | PHE | C3062 | 25.917 | −9.472 | 3.988 | 1.00 | 20.55 | C |
| ATOM | 2303 | CD2 | PHE | C3062 | 26.577 | −9.391 | 6.293 | 1.00 | 20.19 | C |
| ATOM | 2304 | CE1 | PHE | C3062 | 27.200 | −9.883 | 3.622 | 1.00 | 21.03 | C |
| ATOM | 2305 | CE2 | PHE | C3062 | 27.871 | −9.803 | 5.940 | 1.00 | 20.44 | C |
| ATOM | 2306 | CZ | PHE | C3062 | 28.181 | −10.049 | 4.598 | 1.00 | 20.96 | C |
| ATOM | 2307 | C | PHE | C3062 | 24.381 | −6.784 | 4.182 | 1.00 | 20.13 | C |
| ATOM | 2308 | O | PHE | C3062 | 25.474 | −6.231 | 4.037 | 1.00 | 20.04 | O |
| ATOM | 2309 | N | SER | C3063 | 23.530 | −6.981 | 3.174 | 1.00 | 21.29 | N |
| ATOM | 2310 | CA | SER | C3063 | 23.840 | −6.551 | 1.808 | 1.00 | 22.13 | C |
| ATOM | 2311 | CB | SER | C3063 | 22.727 | −6.967 | 0.832 | 1.00 | 22.59 | C |
| ATOM | 2312 | OG | SER | C3063 | 22.699 | −8.374 | 0.636 | 1.00 | 24.36 | O |
| ATOM | 2313 | C | SER | C3063 | 24.019 | −5.032 | 1.762 | 1.00 | 22.61 | C |
| ATOM | 2314 | O | SER | C3063 | 24.913 | −4.527 | 1.082 | 1.00 | 22.10 | O |
| ATOM | 2315 | N | LYS | C3064 | 23.160 | −4.309 | 2.477 | 1.00 | 22.80 | N |
| ATOM | 2316 | CA | LYS | C3064 | 23.257 | −2.852 | 2.530 | 1.00 | 24.07 | C |
| ATOM | 2317 | CB | LYS | C3064 | 22.013 | −2.261 | 3.210 | 1.00 | 23.92 | C |
| ATOM | 2318 | CG | LYS | C3064 | 20.734 | −2.376 | 2.377 | 1.00 | 23.95 | C |
| ATOM | 2319 | CD | LYS | C3064 | 19.505 | −1.886 | 3.138 | 1.00 | 24.12 | C |
| ATOM | 2320 | CE | LYS | C3064 | 19.616 | −0.407 | 3.519 | 1.00 | 24.30 | C |
| ATOM | 2321 | NZ | LYS | C3064 | 18.388 | 0.060 | 4.209 | 1.00 | 24.43 | N |
| ATOM | 2322 | C | LYS | C3064 | 24.519 | −2.408 | 3.281 | 1.00 | 24.84 | C |
| ATOM | 2323 | O | LYS | C3064 | 25.132 | −1.401 | 2.939 | 1.00 | 24.90 | O |
| ATOM | 2324 | N | ALA | C3065 | 24.916 | −3.166 | 4.297 | 1.00 | 26.22 | N |
| ATOM | 2325 | CA | ALA | C3065 | 26.101 | −2.818 | 5.079 | 1.00 | 28.20 | C |
| ATOM | 2326 | CB | ALA | C3065 | 26.184 | −3.693 | 6.324 | 1.00 | 28.57 | C |
| ATOM | 2327 | C | ALA | C3065 | 27.396 | −2.943 | 4.277 | 1.00 | 30.15 | C |
| ATOM | 2328 | O | ALA | C3065 | 28.346 | −2.188 | 4.492 | 1.00 | 29.72 | O |
| ATOM | 2329 | N | MET | C3066 | 27.435 | −3.903 | 3.361 | 1.00 | 32.11 | N |
| ATOM | 2330 | CA | MET | C3066 | 28.629 | −4.113 | 2.555 | 1.00 | 35.57 | C |
| ATOM | 2331 | CB | MET | C3066 | 28.706 | −5.577 | 2.115 | 1.00 | 36.03 | C |
| ATOM | 2332 | CG | MET | C3066 | 30.087 | −5.993 | 1.635 | 1.00 | 38.01 | C |
| ATOM | 2333 | SD | MET | C3066 | 30.589 | −7.586 | 2.315 | 1.00 | 39.43 | S |
| ATOM | 2334 | CE | MET | C3066 | 29.820 | −8.664 | 1.141 | 1.00 | 38.43 | C |
| ATOM | 2335 | C | MET | C3066 | 28.641 | −3.186 | 1.340 | 1.00 | 37.25 | C |
| ATOM | 2336 | O | MET | C3066 | 29.690 | −2.928 | 0.753 | 1.00 | 37.48 | O |
| ATOM | 2337 | N | GLY | C3067 | 27.468 | −2.678 | 0.978 | 1.00 | 39.12 | N |
| ATOM | 2338 | CA | GLY | C3067 | 27.372 | −1.782 | −0.158 | 1.00 | 41.84 | C |
| ATOM | 2339 | C | GLY | C3067 | 27.338 | −2.554 | −1.458 | 1.00 | 43.68 | C |
| ATOM | 2340 | O | GLY | C3067 | 28.251 | −2.446 | −2.277 | 1.00 | 44.30 | O |
| ATOM | 2341 | N | THR | C3068 | 26.281 | −3.337 | −1.648 | 1.00 | 45.16 | N |
| ATOM | 2342 | CA | THR | C3068 | 26.133 | −4.140 | −2.853 | 1.00 | 46.52 | C |
| ATOM | 2343 | CB | THR | C3068 | 27.012 | −5.409 | −2.771 | 1.00 | 46.98 | C |
| ATOM | 2344 | OG1 | THR | C3068 | 26.723 | −6.268 | −3.880 | 1.00 | 47.83 | O |
| ATOM | 2345 | CG2 | THR | C3068 | 26.756 | −6.154 | −1.469 | 1.00 | 47.19 | C |
| ATOM | 2346 | C | THR | C3068 | 24.679 | −4.546 | −3.076 | 1.00 | 47.27 | C |
| ATOM | 2347 | O | THR | C3068 | 23.940 | −4.800 | −2.123 | 1.00 | 47.53 | O |
| ATOM | 2348 | N | GLY | C3069 | 24.272 | −4.597 | −4.341 | 1.00 | 47.97 | N |
| ATOM | 2349 | CA | GLY | C3069 | 22.908 | −4.969 | −4.668 | 1.00 | 48.41 | C |
| ATOM | 2350 | C | GLY | C3069 | 22.848 | −6.140 | −5.629 | 1.00 | 48.94 | C |
| ATOM | 2351 | O | GLY | C3069 | 22.349 | −5.952 | −6.758 | 1.00 | 49.17 | O |
| ATOM | 2352 | OXT | GLY | C3069 | 23.304 | −7.244 | −5.257 | 1.00 | 49.26 | O |
| TER | 2353 | | GLY | C3069 | | | | | | |
| ATOM | 2354 | C | GLY | C3074 | 23.102 | −14.343 | −2.448 | 1.00 | 42.87 | C |
| ATOM | 2355 | O | GLY | C3074 | 23.918 | −14.155 | −1.552 | 1.00 | 43.22 | O |
| ATOM | 2356 | N | GLY | C3074 | 23.249 | −12.549 | −4.163 | 1.00 | 43.05 | N |
| ATOM | 2357 | CA | GLY | C3074 | 23.446 | −14.000 | −3.885 | 1.00 | 42.94 | C |
| ATOM | 2358 | N | PHE | C3075 | 21.892 | −14.845 | −2.228 | 1.00 | 42.82 | N |
| ATOM | 2359 | CA | PHE | C3075 | 21.441 | −15.212 | −0.890 | 1.00 | 42.97 | C |
| ATOM | 2360 | CB | PHE | C3075 | 19.920 | −15.035 | −0.787 | 1.00 | 43.13 | C |
| ATOM | 2361 | CG | PHE | C3075 | 19.484 | −13.638 | −0.400 | 1.00 | 43.82 | C |
| ATOM | 2362 | CD1 | PHE | C3075 | 20.299 | −12.534 | −0.647 | 1.00 | 43.85 | C |
| ATOM | 2363 | CD2 | PHE | C3075 | 18.243 | −13.429 | 0.199 | 1.00 | 43.74 | C |
| ATOM | 2364 | CE1 | PHE | C3075 | 19.883 | −11.243 | −0.304 | 1.00 | 44.03 | C |
| ATOM | 2365 | CE2 | PHE | C3075 | 17.817 | −12.144 | 0.545 | 1.00 | 44.13 | C |
| ATOM | 2366 | CZ | PHE | C3075 | 18.639 | −11.049 | 0.293 | 1.00 | 44.14 | C |
| ATOM | 2367 | C | PHE | C3075 | 21.826 | −16.639 | −0.516 | 1.00 | 42.74 | C |
| ATOM | 2368 | O | PHE | C3075 | 21.820 | −17.007 | 0.661 | 1.00 | 42.92 | O |
| ATOM | 2369 | N | GLN | C3076 | 22.175 | −17.438 | −1.518 | 1.00 | 42.00 | N |
| ATOM | 2370 | CA | GLN | C3076 | 22.548 | −18.824 | −1.276 | 1.00 | 41.00 | C |
| ATOM | 2371 | CB | GLN | C3076 | 22.537 | −19.615 | −2.587 | 1.00 | 41.57 | C |
| ATOM | 2372 | CG | GLN | C3076 | 21.135 | −19.865 | −3.119 | 1.00 | 42.06 | C |
| ATOM | 2373 | CD | GLN | C3076 | 20.286 | −20.695 | −2.171 | 1.00 | 42.45 | C |
| ATOM | 2374 | OE1 | GLN | C3076 | 19.056 | −20.670 | −2.243 | 1.00 | 42.81 | O |
| ATOM | 2375 | NE2 | GLN | C3076 | 20.938 | −21.445 | −1.284 | 1.00 | 42.52 | N |
| ATOM | 2376 | C | GLN | C3076 | 23.892 | −18.977 | −0.584 | 1.00 | 40.27 | C |
| ATOM | 2377 | O | GLN | C3076 | 24.216 | −20.060 | −0.103 | 1.00 | 40.25 | O |
| ATOM | 2378 | N | ASP | C3077 | 24.682 | −17.907 | −0.537 | 1.00 | 38.81 | N |
| ATOM | 2379 | CA | ASP | C3077 | 25.965 | −17.981 | 0.148 | 1.00 | 37.42 | C |
| ATOM | 2380 | CB | ASP | C3077 | 27.107 | −17.527 | −0.769 | 1.00 | 39.03 | C |
| ATOM | 2381 | CG | ASP | C3077 | 27.008 | −16.075 | −1.152 | 1.00 | 40.35 | C |
| ATOM | 2382 | OD1 | ASP | C3077 | 25.897 | −15.635 | −1.514 | 1.00 | 41.46 | O |
| ATOM | 2383 | OD2 | ASP | C3077 | 28.048 | −15.379 | −1.102 | 1.00 | 40.89 | O |
| ATOM | 2384 | C | ASP | C3077 | 25.926 | −17.158 | 1.438 | 1.00 | 35.34 | C |
| ATOM | 2385 | O | ASP | C3077 | 26.916 | −16.556 | 1.848 | 1.00 | 35.66 | O |
| ATOM | 2386 | N | LEU | C3078 | 24.751 | −17.142 | 2.060 | 1.00 | 32.99 | N |
| ATOM | 2387 | CA | LEU | C3078 | 24.513 | −16.456 | 3.328 | 1.00 | 30.22 | C |
| ATOM | 2388 | CB | LEU | C3078 | 23.775 | −15.127 | 3.113 | 1.00 | 30.01 | C |
| ATOM | 2389 | CG | LEU | C3078 | 24.478 | −13.972 | 2.393 | 1.00 | 30.35 | C |
| ATOM | 2390 | CD1 | LEU | C3078 | 23.555 | −12.762 | 2.352 | 1.00 | 29.89 | C |
| ATOM | 2391 | CD2 | LEU | C3078 | 25.768 | −13.615 | 3.114 | 1.00 | 31.03 | C |
| ATOM | 2392 | C | LEU | C3078 | 23.632 | −17.392 | 4.165 | 1.00 | 27.99 | C |
| ATOM | 2393 | O | LEU | C3078 | 22.645 | −17.927 | 3.659 | 1.00 | 27.40 | O |
| ATOM | 2394 | N | GLU | C3079 | 23.984 | −17.589 | 5.435 | 1.00 | 25.61 | N |
| ATOM | 2395 | CA | GLU | C3079 | 23.206 | −18.467 | 6.310 | 1.00 | 24.01 | C |
| ATOM | 2396 | CB | GLU | C3079 | 23.815 | −19.876 | 6.284 | 1.00 | 24.83 | C |
| ATOM | 2397 | CG | GLU | C3079 | 23.137 | −20.897 | 7.190 | 1.00 | 25.45 | C |
| ATOM | 2398 | CD | GLU | C3079 | 23.716 | −22.290 | 7.022 | 1.00 | 26.01 | C |
| ATOM | 2399 | OE1 | GLU | C3079 | 23.270 | −23.022 | 6.110 | 1.00 | 26.62 | O |
| ATOM | 2400 | OE2 | GLU | C3079 | 24.627 | −22.648 | 7.797 | 1.00 | 25.76 | O |
| ATOM | 2401 | C | GLU | C3079 | 23.113 | −17.945 | 7.756 | 1.00 | 22.32 | C |
| ATOM | 2402 | O | GLU | C3079 | 24.113 | −17.500 | 8.335 | 1.00 | 21.78 | O |
| ATOM | 2403 | N | VAL | C3080 | 21.905 | −18.007 | 8.319 | 1.00 | 20.99 | N |
| ATOM | 2404 | CA | VAL | C3080 | 21.616 | −17.554 | 9.685 | 1.00 | 19.93 | C |
| ATOM | 2405 | CB | VAL | C3080 | 20.634 | −16.347 | 9.666 | 1.00 | 19.55 | C |
| ATOM | 2406 | CG1 | VAL | C3080 | 20.302 | −15.911 | 11.099 | 1.00 | 18.41 | C |
| ATOM | 2407 | CG2 | VAL | C3080 | 21.246 | −15.183 | 8.878 | 1.00 | 19.42 | C |
| ATOM | 2408 | C | VAL | C3080 | 20.992 | −18.667 | 10.550 | 1.00 | 20.28 | C |
| ATOM | 2409 | O | VAL | C3080 | 19.888 | −19.140 | 10.261 | 1.00 | 19.70 | O |
| ATOM | 2410 | N | LEU | C3081 | 21.701 | −19.075 | 11.606 | 1.00 | 20.21 | N |
| ATOM | 2411 | CA | LEU | C3081 | 21.232 | −20.121 | 12.521 | 1.00 | 21.05 | C |
| ATOM | 2412 | CB | LEU | C3081 | 22.240 | −21.290 | 12.559 | 1.00 | 21.12 | C |
| ATOM | 2413 | CG | LEU | C3081 | 22.698 | −21.950 | 11.246 | 1.00 | 20.96 | C |
| ATOM | 2414 | CD1 | LEU | C3081 | 23.699 | −23.067 | 11.542 | 1.00 | 21.74 | C |
| ATOM | 2415 | CD2 | LEU | C3081 | 21.497 | −22.498 | 10.489 | 1.00 | 20.34 | C |
| ATOM | 2416 | C | LEU | C3081 | 21.041 | −19.566 | 13.948 | 1.00 | 21.83 | C |
| ATOM | 2417 | O | LEU | C3081 | 21.340 | −18.399 | 14.211 | 1.00 | 20.95 | O |
| ATOM | 2418 | N | ASN | C3082 | 20.546 | −20.411 | 14.855 | 1.00 | 22.96 | N |
| ATOM | 2419 | CA | ASN | C3082 | 20.322 | −20.043 | 16.262 | 1.00 | 24.19 | C |
| ATOM | 2420 | CB | ASN | C3082 | 18.857 | −20.281 | 16.654 | 1.00 | 24.95 | C |
| ATOM | 2421 | CG | ASN | C3082 | 17.913 | −19.223 | 16.101 | 1.00 | 25.21 | C |
| ATOM | 2422 | OD1 | ASN | C3082 | 17.843 | −18.100 | 16.611 | 1.00 | 26.05 | O |
| ATOM | 2423 | ND2 | ASN | C3082 | 17.184 | −19.577 | 15.055 | 1.00 | 24.39 | N |
| ATOM | 2424 | C | ASN | C3082 | 21.212 | −20.890 | 17.184 | 1.00 | 24.76 | C |
| ATOM | 2425 | O | ASN | C3082 | 21.268 | −22.114 | 17.034 | 1.00 | 24.23 | O |
| ATOM | 2426 | N | ASN | C3083 | 21.901 | −20.258 | 18.133 | 1.00 | 24.88 | N |
| ATOM | 2427 | CA | ASN | C3083 | 22.753 | −21.024 | 19.046 | 1.00 | 26.41 | C |
| ATOM | 2428 | CB | ASN | C3083 | 23.880 | −20.147 | 19.619 | 1.00 | 26.57 | C |
| ATOM | 2429 | CG | ASN | C3083 | 23.394 | −19.088 | 20.603 | 1.00 | 27.00 | C |
| ATOM | 2430 | OD1 | ASN | C3083 | 24.185 | −18.255 | 21.047 | 1.00 | 28.80 | O |
| ATOM | 2431 | ND2 | ASN | C3083 | 22.114 | −19.115 | 20.952 | 1.00 | 25.26 | N |
| ATOM | 2432 | C | ASN | C3083 | 21.922 | −21.676 | 20.159 | 1.00 | 26.81 | C |
| ATOM | 2433 | O | ASN | C3083 | 20.697 | −21.536 | 20.178 | 1.00 | 26.49 | O |
| ATOM | 2434 | N | GLU | C3084 | 22.567 | −22.390 | 21.078 | 1.00 | 27.91 | N |
| ATOM | 2435 | CA | GLU | C3084 | 21.815 | −23.070 | 22.132 | 1.00 | 28.75 | C |
| ATOM | 2436 | CB | GLU | C3084 | 22.716 | −24.035 | 22.917 | 1.00 | 29.93 | C |
| ATOM | 2437 | CG | GLU | C3084 | 24.064 | −23.506 | 23.335 | 1.00 | 31.45 | C |
| ATOM | 2438 | CD | GLU | C3084 | 24.916 | −24.594 | 23.966 | 1.00 | 32.85 | C |
| ATOM | 2439 | OE1 | GLU | C3084 | 26.051 | −24.298 | 24.397 | 1.00 | 33.48 | O |
| ATOM | 2440 | OE2 | GLU | C3084 | 24.445 | −25.752 | 24.029 | 1.00 | 33.46 | O |
| ATOM | 2441 | C | GLU | C3084 | 21.024 | −22.189 | 23.092 | 1.00 | 28.50 | C |
| ATOM | 2442 | O | GLU | C3084 | 20.074 | −22.664 | 23.714 | 1.00 | 28.13 | O |
| ATOM | 2443 | N | ARG | C3085 | 21.399 | −20.917 | 23.215 | 1.00 | 28.57 | N |
| ATOM | 2444 | CA | ARG | C3085 | 20.661 | −20.001 | 24.085 | 1.00 | 28.82 | C |
| ATOM | 2445 | CB | ARG | C3085 | 21.584 | −18.929 | 24.670 | 1.00 | 29.93 | C |
| ATOM | 2446 | CG | ARG | C3085 | 22.566 | −19.469 | 25.692 | 1.00 | 31.34 | C |
| ATOM | 2447 | CD | ARG | C3085 | 22.949 | −18.409 | 26.713 | 1.00 | 33.11 | C |
| ATOM | 2448 | NE | ARG | C3085 | 23.902 | −18.936 | 27.683 | 1.00 | 33.67 | N |
| ATOM | 2449 | CZ | ARG | C3085 | 25.167 | −19.217 | 27.398 | 1.00 | 34.16 | C |
| ATOM | 2450 | NH1 | ARG | C3085 | 25.630 | −19.014 | 26.171 | 1.00 | 34.21 | N |
| ATOM | 2451 | NH2 | ARG | C3085 | 25.965 | −19.715 | 28.330 | 1.00 | 33.81 | N |
| ATOM | 2452 | C | ARG | C3085 | 19.519 | −19.332 | 23.318 | 1.00 | 28.44 | C |
| ATOM | 2453 | O | ARG | C3085 | 18.663 | −18.667 | 23.912 | 1.00 | 28.67 | O |
| ATOM | 2454 | N | GLY | C3086 | 19.522 | −19.496 | 21.996 | 1.00 | 27.15 | N |
| ATOM | 2455 | CA | GLY | C3086 | 18.466 | −18.928 | 21.170 | 1.00 | 26.89 | C |
| ATOM | 2456 | C | GLY | C3086 | 18.800 | −17.712 | 20.316 | 1.00 | 26.43 | C |
| ATOM | 2457 | O | GLY | C3086 | 17.942 | −17.229 | 19.581 | 1.00 | 26.94 | O |
| ATOM | 2458 | N | ALA | C3087 | 20.031 | −17.218 | 20.396 | 1.00 | 25.34 | N |
| ATOM | 2459 | CA | ALA | C3087 | 20.432 | −16.043 | 19.622 | 1.00 | 25.00 | C |
| ATOM | 2460 | CB | ALA | C3087 | 21.610 | −15.361 | 20.300 | 1.00 | 25.01 | C |
| ATOM | 2461 | C | ALA | C3087 | 20.788 | −16.355 | 18.163 | 1.00 | 24.71 | C |
| ATOM | 2462 | O | ALA | C3087 | 21.385 | −17.394 | 17.867 | 1.00 | 23.78 | O |
| ATOM | 2463 | N | PRO | C3088 | 20.413 | −15.457 | 17.231 | 1.00 | 23.74 | N |
| ATOM | 2464 | CD | PRO | C3088 | 19.533 | −14.291 | 17.448 | 1.00 | 24.91 | C |
| ATOM | 2465 | CA | PRO | C3088 | 20.697 | −15.631 | 15.800 | 1.00 | 23.12 | C |
| ATOM | 2466 | CB | PRO | C3088 | 19.628 | −14.762 | 15.136 | 1.00 | 24.34 | C |
| ATOM | 2467 | CG | PRO | C3088 | 19.551 | −13.593 | 16.083 | 1.00 | 24.53 | C |
| ATOM | 2468 | C | PRO | C3088 | 22.115 | −15.157 | 15.466 | 1.00 | 22.17 | C |
| ATOM | 2469 | O | PRO | C3088 | 22.606 | −14.198 | 16.070 | 1.00 | 21.42 | O |
| ATOM | 2470 | N | TYR | C3089 | 22.765 | −15.826 | 14.512 | 1.00 | 20.38 | N |
| ATOM | 2471 | CA | TYR | C3089 | 24.128 | −15.469 | 14.098 | 1.00 | 19.72 | C |
| ATOM | 2472 | CB | TYR | C3089 | 25.149 | −16.054 | 15.097 | 1.00 | 19.67 | C |
| ATOM | 2473 | CG | TYR | C3089 | 25.352 | −17.564 | 15.029 | 1.00 | 19.93 | C |
| ATOM | 2474 | CD1 | TYR | C3089 | 26.410 | −18.110 | 14.294 | 1.00 | 21.07 | C |
| ATOM | 2475 | CE1 | TYR | C3089 | 26.594 | −19.497 | 14.194 | 1.00 | 20.45 | C |
| ATOM | 2476 | CD2 | TYR | C3089 | 24.477 | −18.446 | 15.672 | 1.00 | 20.53 | C |
| ATOM | 2477 | CE2 | TYR | C3089 | 24.653 | −19.848 | 15.578 | 1.00 | 20.54 | C |
| ATOM | 2478 | CZ | TYR | C3089 | 25.717 | −20.359 | 14.833 | 1.00 | 20.66 | C |
| ATOM | 2479 | OH | TYR | C3089 | 25.908 | −21.725 | 14.713 | 1.00 | 19.20 | O |
| ATOM | 2480 | C | TYR | C3089 | 24.428 | −15.972 | 12.671 | 1.00 | 19.17 | C |
| ATOM | 2481 | O | TYR | C3089 | 23.820 | −16.944 | 12.213 | 1.00 | 17.72 | O |
| ATOM | 2482 | N | PHE | C3090 | 25.340 | −15.295 | 11.967 | 1.00 | 19.73 | N |
| ATOM | 2483 | CA | PHE | C3090 | 25.729 | −15.704 | 10.607 | 1.00 | 20.26 | C |
| ATOM | 2484 | CB | PHE | C3090 | 26.310 | −14.529 | 9.800 | 1.00 | 20.96 | C |
| ATOM | 2485 | CG | PHE | C3090 | 25.274 | −13.607 | 9.198 | 1.00 | 20.15 | C |
| ATOM | 2486 | CD1 | PHE | C3090 | 24.812 | −12.499 | 9.902 | 1.00 | 20.78 | C |
| ATOM | 2487 | CD2 | PHE | C3090 | 24.791 | −13.831 | 7.912 | 1.00 | 20.37 | C |
| ATOM | 2488 | CE1 | PHE | C3090 | 23.880 | −11.620 | 9.330 | 1.00 | 20.73 | C |
| ATOM | 2489 | CE2 | PHE | C3090 | 23.860 | −12.964 | 7.326 | 1.00 | 20.67 | C |
| ATOM | 2490 | CZ | PHE | C3090 | 23.404 | −11.853 | 8.040 | 1.00 | 20.35 | C |
| ATOM | 2491 | C | PHE | C3090 | 26.788 | −16.812 | 10.637 | 1.00 | 20.91 | C |
| ATOM | 2492 | O | PHE | C3090 | 27.905 | −16.598 | 11.117 | 1.00 | 20.54 | O |
| ATOM | 2493 | N | SER | C3091 | 26.442 | −17.986 | 10.116 | 1.00 | 21.82 | N |
| ATOM | 2494 | CA | SER | C3091 | 27.378 | −19.113 | 10.073 | 1.00 | 22.73 | C |
| ATOM | 2495 | CB | SER | C3091 | 26.630 | −20.435 | 10.290 | 1.00 | 22.96 | C |
| ATOM | 2496 | OG | SER | C3091 | 25.613 | −20.617 | 9.318 | 1.00 | 23.28 | O |
| ATOM | 2497 | C | SER | C3091 | 28.137 | −19.164 | 8.738 | 1.00 | 23.33 | C |
| ATOM | 2498 | O | SER | C3091 | 29.114 | −19.904 | 8.604 | 1.00 | 22.86 | O |
| ATOM | 2499 | N | GLN | C3092 | 27.683 | −18.375 | 7.762 | 1.00 | 23.62 | N |
| ATOM | 2500 | CA | GLN | C3092 | 28.309 | −18.319 | 6.436 | 1.00 | 24.85 | C |
| ATOM | 2501 | CB | GLN | C3092 | 27.730 | −19.439 | 5.558 | 1.00 | 25.88 | C |
| ATOM | 2502 | CG | GLN | C3092 | 28.045 | −19.357 | 4.064 | 1.00 | 28.11 | C |
| ATOM | 2503 | CD | GLN | C3092 | 29.529 | −19.339 | 3.762 | 1.00 | 29.40 | C |
| ATOM | 2504 | OE1 | GLN | C3092 | 30.298 | −20.131 | 4.310 | 1.00 | 31.43 | O |
| ATOM | 2505 | NE2 | GLN | C3092 | 29.940 | −18.441 | 2.874 | 1.00 | 30.15 | N |
| ATOM | 2506 | C | GLN | C3092 | 28.107 | −16.944 | 5.764 | 1.00 | 24.73 | C |
| ATOM | 2507 | O | GLN | C3092 | 26.974 | −16.472 | 5.637 | 1.00 | 24.71 | O |
| ATOM | 2508 | N | ALA | C3093 | 29.205 | −16.310 | 5.343 | 1.00 | 23.94 | N |
| ATOM | 2509 | CA | ALA | C3093 | 29.160 | −14.994 | 4.685 | 1.00 | 23.97 | C |
| ATOM | 2510 | CB | ALA | C3093 | 28.779 | −13.909 | 5.706 | 1.00 | 23.95 | C |
| ATOM | 2511 | C | ALA | C3093 | 30.493 | −14.618 | 4.009 | 1.00 | 23.83 | C |
| ATOM | 2512 | O | ALA | C3093 | 31.556 | −15.008 | 4.472 | 1.00 | 23.82 | O |
| ATOM | 2513 | N | PRO | C3094 | 30.443 | −13.841 | 2.910 | 1.00 | 24.15 | N |
| ATOM | 2514 | CD | PRO | C3094 | 29.221 | −13.410 | 2.203 | 1.00 | 24.15 | C |
| ATOM | 2515 | CA | PRO | C3094 | 31.649 | −13.414 | 2.177 | 1.00 | 24.68 | C |
| ATOM | 2516 | CB | PRO | C3094 | 31.098 | −13.058 | 0.801 | 1.00 | 24.26 | C |
| ATOM | 2517 | CG | PRO | C3094 | 29.762 | −12.461 | 1.144 | 1.00 | 24.90 | C |
| ATOM | 2518 | C | PRO | C3094 | 32.369 | −12.231 | 2.840 | 1.00 | 24.92 | C |
| ATOM | 2519 | O | PRO | C3094 | 32.529 | −11.165 | 2.239 | 1.00 | 25.65 | O |
| ATOM | 2520 | N | PHE | C3095 | 32.810 | −12.437 | 4.076 | 1.00 | 25.08 | N |
| ATOM | 2521 | CA | PHE | C3095 | 33.495 | −11.410 | 4.864 | 1.00 | 25.37 | C |
| ATOM | 2522 | CB | PHE | C3095 | 32.444 | −10.502 | 5.521 | 1.00 | 24.79 | C |
| ATOM | 2523 | CG | PHE | C3095 | 33.015 | −9.384 | 6.356 | 1.00 | 24.93 | C |
| ATOM | 2524 | CD1 | PHE | C3095 | 33.652 | −8.299 | 5.760 | 1.00 | 24.60 | C |
| ATOM | 2525 | CD2 | PHE | C3095 | 32.883 | −9.403 | 7.741 | 1.00 | 24.43 | C |
| ATOM | 2526 | CE1 | PHE | C3095 | 34.148 | −7.247 | 6.536 | 1.00 | 24.72 | C |
| ATOM | 2527 | CE2 | PHE | C3095 | 33.374 | −8.358 | 8.527 | 1.00 | 24.22 | C |
| ATOM | 2528 | CZ | PHE | C3095 | 34.008 | −7.278 | 7.921 | 1.00 | 24.70 | C |
| ATOM | 2529 | C | PHE | C3095 | 34.288 | −12.163 | 5.928 | 1.00 | 25.61 | C |
| ATOM | 2530 | O | PHE | C3095 | 33.766 | −13.101 | 6.537 | 1.00 | 26.06 | O |
| ATOM | 2531 | N | SER | C3096 | 35.538 | −11.773 | 6.164 | 1.00 | 26.42 | N |
| ATOM | 2532 | CA | SER | C3096 | 36.343 | −12.485 | 7.158 | 1.00 | 27.23 | C |
| ATOM | 2533 | CB | SER | C3096 | 37.709 | −12.857 | 6.558 | 1.00 | 27.32 | C |
| ATOM | 2534 | OG | SER | C3096 | 38.389 | −11.725 | 6.054 | 1.00 | 28.68 | O |
| ATOM | 2535 | C | SER | C3096 | 36.529 | −11.795 | 8.515 | 1.00 | 27.58 | C |
| ATOM | 2536 | O | SER | C3096 | 37.311 | −12.259 | 9.349 | 1.00 | 28.21 | O |
| ATOM | 2537 | N | GLY | C3097 | 35.807 | −10.700 | 8.745 | 1.00 | 27.18 | N |
| ATOM | 2538 | CA | GLY | C3097 | 35.908 | −10.003 | 10.021 | 1.00 | 26.33 | C |
| ATOM | 2539 | C | GLY | C3097 | 34.808 | −10.462 | 10.971 | 1.00 | 26.17 | C |
| ATOM | 2540 | O | GLY | C3097 | 34.322 | −11.584 | 10.845 | 1.00 | 25.72 | O |
| ATOM | 2541 | N | LYS | C3098 | 34.408 | −9.610 | 11.916 | 1.00 | 25.25 | N |
| ATOM | 2542 | CA | LYS | C3098 | 33.347 | −9.971 | 12.864 | 1.00 | 24.54 | C |
| ATOM | 2543 | CB | LYS | C3098 | 33.759 | −9.591 | 14.289 | 1.00 | 25.96 | C |
| ATOM | 2544 | CG | LYS | C3098 | 35.059 | −10.259 | 14.723 | 1.00 | 28.19 | C |
| ATOM | 2545 | CD | LYS | C3098 | 35.079 | −10.576 | 16.208 | 1.00 | 29.56 | C |
| ATOM | 2546 | CE | LYS | C3098 | 35.022 | −9.324 | 17.053 | 1.00 | 30.68 | C |
| ATOM | 2547 | NZ | LYS | C3098 | 35.150 | −9.647 | 18.505 | 1.00 | 32.82 | N |
| ATOM | 2548 | C | LYS | C3098 | 32.013 | −9.313 | 12.498 | 1.00 | 22.87 | C |
| ATOM | 2549 | O | LYS | C3098 | 31.984 | −8.155 | 12.082 | 1.00 | 22.97 | O |
| ATOM | 2550 | N | ILE | C3099 | 30.919 | −10.060 | 12.659 | 1.00 | 21.29 | N |
| ATOM | 2551 | CA | ILE | C3099 | 29.567 | −9.589 | 12.316 | 1.00 | 19.46 | C |
| ATOM | 2552 | CB | ILE | C3099 | 28.918 | −10.546 | 11.251 | 1.00 | 19.44 | C |
| ATOM | 2553 | CG2 | ILE | C3099 | 27.577 | −9.987 | 10.769 | 1.00 | 18.38 | C |
| ATOM | 2554 | CG1 | ILE | C3099 | 29.869 | −10.742 | 10.062 | 1.00 | 19.30 | C |
| ATOM | 2555 | CD1 | ILE | C3099 | 29.447 | −11.853 | 9.103 | 1.00 | 19.40 | C |
| ATOM | 2556 | C | ILE | C3099 | 28.632 | −9.537 | 13.544 | 1.00 | 18.29 | C |
| ATOM | 2557 | O | ILE | C3099 | 28.381 | −10.568 | 14.165 | 1.00 | 18.68 | O |
| ATOM | 2558 | N | TRP | C3100 | 28.109 | −8.357 | 13.888 | 1.00 | 16.88 | N |
| ATOM | 2559 | CA | TRP | C3100 | 27.195 | −8.233 | 15.036 | 1.00 | 15.59 | C |
| ATOM | 2560 | CB | TRP | C3100 | 27.643 | −7.078 | 15.956 | 1.00 | 15.65 | C |
| ATOM | 2561 | CG | TRP | C3100 | 29.076 | −7.231 | 16.505 | 1.00 | 15.70 | C |
| ATOM | 2562 | CD2 | TRP | C3100 | 29.492 | −8.034 | 17.626 | 1.00 | 15.13 | C |
| ATOM | 2563 | CE2 | TRP | C3100 | 30.901 | −7.910 | 17.735 | 1.00 | 15.28 | C |
| ATOM | 2564 | CE3 | TRP | C3100 | 28.813 | −8.846 | 18.547 | 1.00 | 16.40 | C |
| ATOM | 2565 | CD1 | TRP | C3100 | 30.220 | −6.668 | 15.997 | 1.00 | 15.61 | C |
| ATOM | 2566 | NE1 | TRP | C3100 | 31.321 | −7.074 | 16.734 | 1.00 | 15.83 | N |
| ATOM | 2567 | CZ2 | TRP | C3100 | 31.639 | −8.570 | 18.730 | 1.00 | 15.21 | C |
| ATOM | 2568 | CZ3 | TRP | C3100 | 29.549 | −9.504 | 19.538 | 1.00 | 15.48 | C |
| ATOM | 2569 | CH2 | TRP | C3100 | 30.950 | −9.358 | 19.618 | 1.00 | 15.73 | C |
| ATOM | 2570 | C | TRP | C3100 | 25.746 | −8.022 | 14.533 | 1.00 | 15.44 | C |
| ATOM | 2571 | O | TRP | C3100 | 25.438 | −6.991 | 13.924 | 1.00 | 14.84 | O |
| ATOM | 2572 | N | LEU | C3101 | 24.875 | −9.003 | 14.800 | 1.00 | 14.06 | N |
| ATOM | 2573 | CA | LEU | C3101 | 23.469 | −9.004 | 14.348 | 1.00 | 13.05 | C |
| ATOM | 2574 | CB | LEU | C3101 | 23.268 | −10.161 | 13.357 | 1.00 | 13.07 | C |
| ATOM | 2575 | CG | LEU | C3101 | 21.828 | −10.623 | 13.084 | 1.00 | 12.96 | C |
| ATOM | 2576 | CD1 | LEU | C3101 | 21.208 | −9.737 | 12.015 | 1.00 | 13.42 | C |
| ATOM | 2577 | CD2 | LEU | C3101 | 21.821 | −12.083 | 12.624 | 1.00 | 14.82 | C |
| ATOM | 2578 | C | LEU | C3101 | 22.382 | −9.133 | 15.430 | 1.00 | 12.57 | C |
| ATOM | 2579 | O | LEU | C3101 | 22.597 | −9.789 | 16.450 | 1.00 | 11.63 | O |
| ATOM | 2580 | N | SER | C3102 | 21.215 | −8.525 | 15.181 | 1.00 | 12.95 | N |
| ATOM | 2581 | CA | SER | C3102 | 20.054 | −8.615 | 16.092 | 1.00 | 13.03 | C |
| ATOM | 2582 | CB | SER | C3102 | 20.128 | −7.536 | 17.187 | 1.00 | 13.35 | C |
| ATOM | 2583 | OG | SER | C3102 | 19.148 | −7.749 | 18.205 | 1.00 | 12.78 | O |
| ATOM | 2584 | C | SER | C3102 | 18.736 | −8.484 | 15.297 | 1.00 | 13.43 | C |
| ATOM | 2585 | O | SER | C3102 | 18.687 | −7.747 | 14.298 | 1.00 | 13.38 | O |
| ATOM | 2586 | N | ILE | C3103 | 17.687 | −9.197 | 15.741 | 1.00 | 13.23 | N |
| ATOM | 2587 | CA | ILE | C3103 | 16.355 | −9.215 | 15.082 | 1.00 | 14.65 | C |
| ATOM | 2588 | CB | ILE | C3103 | 16.135 | −10.552 | 14.290 | 1.00 | 15.02 | C |
| ATOM | 2589 | CG2 | ILE | C3103 | 14.776 | −10.534 | 13.581 | 1.00 | 14.81 | C |
| ATOM | 2590 | CG1 | ILE | C3103 | 17.254 | −10.764 | 13.267 | 1.00 | 15.40 | C |
| ATOM | 2591 | CD1 | ILE | C3103 | 17.113 | −12.053 | 12.443 | 1.00 | 16.79 | C |
| ATOM | 2592 | C | ILE | C3103 | 15.180 | −9.123 | 16.087 | 1.00 | 15.28 | C |
| ATOM | 2593 | O | ILE | C3103 | 15.239 | −9.717 | 17.166 | 1.00 | 15.06 | O |
| ATOM | 2594 | N | SER | C3104 | 14.109 | −8.414 | 15.726 | 1.00 | 16.55 | N |
| ATOM | 2595 | CA | SER | C3104 | 12.930 | −8.295 | 16.614 | 1.00 | 17.82 | C |
| ATOM | 2596 | CB | SER | C3104 | 13.077 | −7.067 | 17.527 | 1.00 | 18.38 | C |
| ATOM | 2597 | OG | SER | C3104 | 12.058 | −7.028 | 18.524 | 1.00 | 18.13 | O |
| ATOM | 2598 | C | SER | C3104 | 11.629 | −8.186 | 15.793 | 1.00 | 19.18 | C |
| ATOM | 2599 | O | SER | C3104 | 11.672 | −7.850 | 14.608 | 1.00 | 18.60 | O |
| ATOM | 2600 | N | HIS | C3105 | 10.475 | −8.466 | 16.405 | 1.00 | 21.28 | N |
| ATOM | 2601 | CA | HIS | C3105 | 9.209 | −8.376 | 15.660 | 1.00 | 23.07 | C |
| ATOM | 2602 | CB | HIS | C3105 | 8.964 | −9.664 | 14.877 | 1.00 | 24.36 | C |
| ATOM | 2603 | CG | HIS | C3105 | 8.715 | −10.856 | 15.749 | 1.00 | 25.51 | C |
| ATOM | 2604 | CD2 | HIS | C3105 | 7.631 | −11.219 | 16.474 | 1.00 | 26.93 | C |
| ATOM | 2605 | ND1 | HIS | C3105 | 9.673 | −11.815 | 15.986 | 1.00 | 26.74 | N |
| ATOM | 2606 | CE1 | HIS | C3105 | 9.192 | −12.721 | 16.820 | 1.00 | 27.20 | C |
| ATOM | 2607 | NE2 | HIS | C3105 | 7.955 | −12.383 | 17.132 | 1.00 | 28.00 | N |
| ATOM | 2608 | C | HIS | C3105 | 7.927 | −8.081 | 16.452 | 1.00 | 24.09 | C |
| ATOM | 2609 | O | HIS | C3105 | 7.913 | −8.058 | 17.686 | 1.00 | 23.14 | O |
| ATOM | 2610 | N | THR | C3106 | 6.851 | −7.870 | 15.692 | 1.00 | 25.15 | N |
| ATOM | 2611 | CA | THR | C3106 | 5.506 | −7.616 | 16.212 | 1.00 | 26.55 | C |
| ATOM | 2612 | CB | THR | C3106 | 5.100 | −6.137 | 16.097 | 1.00 | 26.80 | C |
| ATOM | 2613 | OG1 | THR | C3106 | 5.097 | −5.748 | 14.717 | 1.00 | 27.05 | O |
| ATOM | 2614 | CG2 | THR | C3106 | 6.052 | −5.258 | 16.880 | 1.00 | 27.26 | C |
| ATOM | 2615 | C | THR | C3106 | 4.549 | −8.437 | 15.339 | 1.00 | 27.26 | C |
| ATOM | 2616 | O | THR | C3106 | 4.987 | −9.196 | 14.477 | 1.00 | 26.93 | O |
| ATOM | 2617 | N | ASP | C3107 | 3.245 | −8.287 | 15.539 | 1.00 | 28.56 | N |
| ATOM | 2618 | CA | ASP | C3107 | 2.310 | −9.067 | 14.732 | 1.00 | 30.14 | C |
| ATOM | 2619 | CB | ASP | C3107 | 0.977 | −9.243 | 15.474 | 1.00 | 32.49 | C |
| ATOM | 2620 | CG | ASP | C3107 | 0.498 | −7.968 | 16.133 | 1.00 | 34.70 | C |
| ATOM | 2621 | OD1 | ASP | C3107 | 0.406 | −6.930 | 15.440 | 1.00 | 37.09 | O |
| ATOM | 2622 | OD2 | ASP | C3107 | 0.209 | −8.003 | 17.348 | 1.00 | 36.52 | O |
| ATOM | 2623 | C | ASP | C3107 | 2.065 | −8.499 | 13.334 | 1.00 | 29.74 | C |
| ATOM | 2624 | O | ASP | C3107 | 1.264 | −9.043 | 12.575 | 1.00 | 30.83 | O |
| ATOM | 2625 | N | GLN | C3108 | 2.762 | −7.425 | 12.979 | 1.00 | 28.55 | N |
| ATOM | 2626 | CA | GLN | C3108 | 2.578 | −6.815 | 11.665 | 1.00 | 27.66 | C |
| ATOM | 2627 | CB | GLN | C3108 | 1.942 | −5.432 | 11.822 | 1.00 | 27.82 | C |
| ATOM | 2628 | CG | GLN | C3108 | 0.557 | −5.445 | 12.466 | 1.00 | 28.58 | C |
| ATOM | 2629 | CD | GLN | C3108 | 0.012 | −4.045 | 12.681 | 0.05 | 28.18 | C |
| ATOM | 2630 | OE1 | GLN | C3108 | −1.083 | −3.866 | 13.214 | 0.05 | 28.21 | O |
| ATOM | 2631 | NE2 | GLN | C3108 | 0.779 | −3.043 | 12.266 | 0.05 | 28.20 | N |
| ATOM | 2632 | C | GLN | C3108 | 3.845 | −6.693 | 10.812 | 1.00 | 26.77 | C |
| ATOM | 2633 | O | GLN | C3108 | 3.760 | −6.652 | 9.582 | 1.00 | 25.82 | O |
| ATOM | 2634 | N | PHE | C3109 | 5.015 | −6.625 | 11.449 | 1.00 | 25.68 | N |
| ATOM | 2635 | CA | PHE | C3109 | 6.268 | −6.496 | 10.697 | 1.00 | 24.08 | C |
| ATOM | 2636 | CB | PHE | C3109 | 6.441 | −5.039 | 10.221 | 1.00 | 25.01 | C |
| ATOM | 2637 | CG | PHE | C3109 | 6.572 | −4.029 | 11.343 | 1.00 | 26.02 | C |
| ATOM | 2638 | CD1 | PHE | C3109 | 7.763 | −3.908 | 12.062 | 1.00 | 26.89 | C |
| ATOM | 2639 | CD2 | PHE | C3109 | 5.505 | −3.205 | 11.682 | 1.00 | 26.71 | C |
| ATOM | 2640 | CE1 | PHE | C3109 | 7.889 | −2.977 | 13.105 | 1.00 | 27.26 | C |
| ATOM | 2641 | CE2 | PHE | C3109 | 5.616 | −2.268 | 12.725 | 1.00 | 27.81 | C |
| ATOM | 2642 | CZ | PHE | C3109 | 6.811 | −2.156 | 13.437 | 1.00 | 27.24 | C |
| ATOM | 2643 | C | PHE | C3109 | 7.497 | −6.950 | 11.497 | 1.00 | 22.66 | C |
| ATOM | 2644 | O | PHE | C3109 | 7.382 | −7.267 | 12.681 | 1.00 | 21.87 | O |
| ATOM | 2645 | N | VAL | C3110 | 8.658 | −6.992 | 10.833 | 1.00 | 20.94 | N |
| ATOM | 2646 | CA | VAL | C3110 | 9.932 | −7.396 | 11.460 | 1.00 | 19.58 | C |
| ATOM | 2647 | CB | VAL | C3110 | 10.402 | −8.790 | 10.931 | 1.00 | 20.14 | C |
| ATOM | 2648 | CG1 | VAL | C3110 | 10.731 | −8.705 | 9.443 | 1.00 | 20.40 | C |
| ATOM | 2649 | CG2 | VAL | C3110 | 11.639 | −9.268 | 11.707 | 1.00 | 21.57 | C |
| ATOM | 2650 | C | VAL | C3110 | 11.049 | −6.374 | 11.162 | 1.00 | 18.29 | C |
| ATOM | 2651 | O | VAL | C3110 | 10.982 | −5.675 | 10.154 | 1.00 | 16.70 | O |
| ATOM | 2652 | N | THR | C3111 | 12.065 | −6.298 | 12.035 | 1.00 | 17.21 | N |
| ATOM | 2653 | CA | THR | C3111 | 13.210 | −5.383 | 11.852 | 1.00 | 16.21 | C |
| ATOM | 2654 | CB | THR | C3111 | 13.109 | −4.122 | 12.781 | 1.00 | 16.74 | C |
| ATOM | 2655 | OG1 | THR | C3111 | 13.100 | −4.528 | 14.159 | 1.00 | 19.56 | O |
| ATOM | 2656 | CG2 | THR | C3111 | 11.851 | −3.321 | 12.485 | 1.00 | 18.23 | C |
| ATOM | 2657 | C | THR | C3111 | 14.551 | −6.096 | 12.159 | 1.00 | 15.10 | C |
| ATOM | 2658 | O | THR | C3111 | 14.575 | −7.053 | 12.945 | 1.00 | 13.98 | O |
| ATOM | 2659 | N | ALA | C3112 | 15.643 | −5.638 | 11.532 | 1.00 | 13.72 | N |
| ATOM | 2660 | CA | ALA | C3112 | 16.996 | −6.210 | 11.737 | 1.00 | 14.06 | C |
| ATOM | 2661 | CB | ALA | C3112 | 17.266 | −7.324 | 10.712 | 1.00 | 13.98 | C |
| ATOM | 2662 | C | ALA | C3112 | 18.092 | −5.135 | 11.630 | 1.00 | 13.79 | C |
| ATOM | 2663 | O | ALA | C3112 | 17.936 | −4.164 | 10.884 | 1.00 | 14.41 | O |
| ATOM | 2664 | N | SER | C3113 | 19.194 | −5.320 | 12.364 | 1.00 | 13.18 | N |
| ATOM | 2665 | CA | SER | C3113 | 20.321 | −4.369 | 12.395 | 1.00 | 12.99 | C |
| ATOM | 2666 | CB | SER | C3113 | 20.210 | −3.510 | 13.665 | 1.00 | 12.64 | C |
| ATOM | 2667 | OG | SER | C3113 | 21.209 | −2.499 | 13.724 | 1.00 | 12.28 | O |
| ATOM | 2668 | C | SER | C3113 | 21.690 | −5.103 | 12.369 | 1.00 | 13.25 | C |
| ATOM | 2669 | O | SER | C3113 | 21.875 | −6.075 | 13.097 | 1.00 | 12.44 | O |
| ATOM | 2670 | N | VAL | C3114 | 22.636 | −4.611 | 11.555 | 1.00 | 13.63 | N |
| ATOM | 2671 | CA | VAL | C3114 | 23.972 | −5.230 | 11.374 | 1.00 | 13.14 | C |
| ATOM | 2672 | CB | VAL | C3114 | 24.030 | −5.984 | 9.989 | 1.00 | 12.86 | C |
| ATOM | 2673 | CG1 | VAL | C3114 | 25.467 | −6.425 | 9.641 | 1.00 | 13.59 | C |
| ATOM | 2674 | CG2 | VAL | C3114 | 23.117 | −7.194 | 10.016 | 1.00 | 13.11 | C |
| ATOM | 2675 | C | VAL | C3114 | 25.161 | −4.248 | 11.419 | 1.00 | 13.18 | C |
| ATOM | 2676 | O | VAL | C3114 | 25.094 | −3.171 | 10.834 | 1.00 | 11.64 | O |
| ATOM | 2677 | N | ILE | C3115 | 26.245 | −4.631 | 12.101 | 1.00 | 14.05 | N |
| ATOM | 2678 | CA | ILE | C3115 | 27.463 | −3.797 | 12.176 | 1.00 | 15.16 | C |
| ATOM | 2679 | CB | ILE | C3115 | 27.717 | −3.208 | 13.593 | 1.00 | 15.92 | C |
| ATOM | 2680 | CG2 | ILE | C3115 | 28.902 | −2.217 | 13.532 | 1.00 | 16.68 | C |
| ATOM | 2681 | CG1 | ILE | C3115 | 26.482 | −2.477 | 14.122 | 1.00 | 15.85 | C |
| ATOM | 2682 | CD1 | ILE | C3115 | 26.657 | −1.949 | 15.549 | 1.00 | 15.56 | C |
| ATOM | 2683 | C | ILE | C3115 | 28.699 | −4.655 | 11.836 | 1.00 | 16.02 | C |
| ATOM | 2684 | O | ILE | C3115 | 28.900 | −5.714 | 12.444 | 1.00 | 14.67 | O |
| ATOM | 2685 | N | LEU | C3116 | 29.519 | −4.195 | 10.883 | 1.00 | 16.42 | N |
| ATOM | 2686 | CA | LEU | C3116 | 30.741 | −4.920 | 10.469 | 1.00 | 17.73 | C |
| ATOM | 2687 | CB | LEU | C3116 | 30.864 | −4.910 | 8.939 | 1.00 | 17.22 | C |
| ATOM | 2688 | CG | LEU | C3116 | 29.646 | −5.423 | 8.157 | 1.00 | 17.27 | C |
| ATOM | 2689 | CD1 | LEU | C3116 | 29.914 | −5.349 | 6.660 | 1.00 | 17.70 | C |
| ATOM | 2690 | CD2 | LEU | C3116 | 29.339 | −6.855 | 8.570 | 1.00 | 18.07 | C |
| ATOM | 2691 | C | LEU | C3116 | 32.016 | −4.322 | 11.099 | 1.00 | 18.48 | C |
| ATOM | 2692 | O | LEU | C3116 | 32.185 | −3.101 | 11.134 | 1.00 | 16.07 | O |
| ATOM | 2693 | N | GLU | C3117 | 32.926 | −5.184 | 11.562 | 1.00 | 20.56 | N |
| ATOM | 2694 | CA | GLU | C3117 | 34.150 | −4.718 | 12.227 | 1.00 | 23.53 | C |
| ATOM | 2695 | CB | GLU | C3117 | 33.939 | −4.757 | 13.754 | 1.00 | 24.06 | C |
| ATOM | 2696 | CG | GLU | C3117 | 35.200 | −4.587 | 14.608 | 1.00 | 24.76 | C |
| ATOM | 2697 | CD | GLU | C3117 | 34.952 | −4.835 | 16.103 | 1.00 | 25.52 | C |
| ATOM | 2698 | OE1 | GLU | C3117 | 34.507 | −5.946 | 16.473 | 1.00 | 25.65 | O |
| ATOM | 2699 | OE2 | GLU | C3117 | 35.203 | −3.917 | 16.913 | 1.00 | 25.88 | O |
| ATOM | 2700 | C | GLU | C3117 | 35.429 | −5.488 | 11.878 | 1.00 | 25.38 | C |
| ATOM | 2701 | O | GLU | C3117 | 35.403 | −6.695 | 11.654 | 1.00 | 25.28 | O |
| ATOM | 2702 | N | GLU | C3118 | 36.545 | −4.766 | 11.840 | 1.00 | 28.60 | N |
| ATOM | 2703 | CA | GLU | C3118 | 37.857 | −5.346 | 11.559 | 1.00 | 32.23 | C |
| ATOM | 2704 | CB | GLU | C3118 | 38.321 | −4.996 | 10.139 | 1.00 | 33.43 | C |
| ATOM | 2705 | CG | GLU | C3118 | 37.520 | −5.678 | 9.038 | 1.00 | 35.47 | C |
| ATOM | 2706 | CD | GLU | C3118 | 38.123 | −5.482 | 7.657 | 1.00 | 36.73 | C |
| ATOM | 2707 | OE1 | GLU | C3118 | 37.561 | −6.029 | 6.683 | 1.00 | 37.62 | O |
| ATOM | 2708 | OE2 | GLU | C3118 | 39.156 | −4.786 | 7.540 | 1.00 | 37.51 | O |
| ATOM | 2709 | C | GLU | C3118 | 38.857 | −4.800 | 12.576 | 1.00 | 33.91 | C |
| ATOM | 2710 | O | GLU | C3118 | 39.168 | −3.608 | 12.576 | 1.00 | 34.12 | O |
| ATOM | 2711 | N | ASN | C3119 | 39.348 | −5.678 | 13.444 | 1.00 | 36.07 | N |
| ATOM | 2712 | CA | ASN | C3119 | 40.308 | −5.298 | 14.477 | 1.00 | 37.92 | C |
| ATOM | 2713 | CB | ASN | C3119 | 40.307 | −6.343 | 15.597 | 1.00 | 38.97 | C |
| ATOM | 2714 | CG | ASN | C3119 | 38.908 | −6.622 | 16.137 | 1.00 | 39.98 | C |
| ATOM | 2715 | OD1 | ASN | C3119 | 38.271 | −5.753 | 16.737 | 1.00 | 40.51 | O |
| ATOM | 2716 | ND2 | ASN | C3119 | 38.424 | −7.841 | 15.919 | 1.00 | 40.65 | N |
| ATOM | 2717 | C | ASN | C3119 | 41.713 | −5.169 | 13.889 | 1.00 | 38.71 | C |
| ATOM | 2718 | O | ASN | C3119 | 41.850 | −5.354 | 12.662 | 1.00 | 39.38 | O |
| ATOM | 2719 | OXT | ASN | C3119 | 42.661 | −4.885 | 14.657 | 1.00 | 39.94 | O |
| TER | 2720 | | ASN | C3119 | | | | | | |
| ATOM | 2721 | O | HOH | W5001 | 1.203 | 5.537 | 8.026 | 1.00 | 17.73 | O |
| ATOM | 2722 | O | HOH | W5002 | 14.815 | 9.909 | 2.322 | 1.00 | 14.32 | O |
| ATOM | 2723 | O | HOH | W5003 | 23.904 | 8.622 | 37.957 | 1.00 | 27.02 | O |
| ATOM | 2724 | O | HOH | W5004 | 20.469 | 3.444 | 3.735 | 1.00 | 17.55 | O |
| ATOM | 2725 | O | HOH | W5005 | 24.018 | 4.203 | 28.855 | 1.00 | 20.10 | O |
| ATOM | 2726 | O | HOH | W5006 | 10.973 | 5.376 | 28.684 | 1.00 | 19.79 | O |
| ATOM | 2727 | O | HOH | W5007 | 22.784 | 15.314 | 34.546 | 1.00 | 22.90 | O |
| ATOM | 2728 | O | HOH | W5008 | 23.453 | 4.929 | 3.777 | 1.00 | 19.63 | O |
| ATOM | 2729 | O | HOH | W5009 | 23.062 | 1.129 | 4.843 | 1.00 | 21.68 | O |
| ATOM | 2730 | O | HOH | W5010 | 25.642 | 18.163 | 37.608 | 1.00 | 19.94 | O |
| ATOM | 2731 | O | HOH | W5011 | 23.184 | 2.653 | 2.606 | 1.00 | 26.65 | O |
| ATOM | 2732 | O | HOH | W5012 | 28.019 | 0.947 | 5.252 | 1.00 | 22.66 | O |
| ATOM | 2733 | O | HOH | W5013 | 4.711 | 21.958 | 2.912 | 1.00 | 24.85 | O |
| ATOM | 2734 | O | HOH | W5014 | 36.483 | 3.458 | 10.706 | 1.00 | 31.97 | O |
| ATOM | 2735 | O | HOH | W5015 | 17.241 | 0.177 | 14.610 | 1.00 | 23.93 | O |
| ATOM | 2736 | O | HOH | W5016 | 13.566 | −15.296 | 11.672 | 1.00 | 22.95 | O |
| ATOM | 2737 | O | HOH | W5017 | 19.479 | 14.186 | −2.520 | 1.00 | 25.49 | O |
| ATOM | 2738 | O | HOH | W5018 | 27.283 | 10.084 | 6.422 | 1.00 | 24.49 | O |
| ATOM | 2739 | O | HOH | W5019 | 14.135 | 8.932 | 34.920 | 1.00 | 26.53 | O |
| ATOM | 2740 | O | HOH | W5020 | 36.044 | 2.408 | 16.583 | 1.00 | 32.56 | O |
| ATOM | 2741 | O | HOH | W5021 | 33.258 | −17.363 | 3.678 | 1.00 | 19.73 | O |
| ATOM | 2742 | O | HOH | W5022 | 15.667 | −0.884 | 2.725 | 1.00 | 31.95 | O |
| ATOM | 2743 | O | HOH | W5023 | 9.740 | −0.876 | 42.696 | 1.00 | 26.45 | O |
| ATOM | 2744 | O | HOH | W5024 | 28.298 | −24.311 | 26.736 | 1.00 | 36.28 | O |
| ATOM | 2745 | O | HOH | W5025 | 16.170 | 2.461 | 13.853 | 1.00 | 22.79 | O |
| ATOM | 2746 | O | HOH | W5026 | 13.623 | −1.972 | 15.922 | 1.00 | 33.10 | O |
| ATOM | 2747 | O | HOH | W5027 | 35.568 | −7.137 | 18.918 | 1.00 | 28.33 | O |
| ATOM | 2748 | O | HOH | W5028 | 21.193 | 8.337 | 22.471 | 1.00 | 24.39 | O |
| ATOM | 2749 | O | HOH | W5029 | 0.905 | 8.530 | 3.669 | 1.00 | 29.99 | O |
| ATOM | 2750 | O | HOH | W5030 | 26.791 | 21.664 | 30.504 | 1.00 | 26.91 | O |
| ATOM | 2751 | O | HOH | W5031 | 12.687 | −4.279 | 3.191 | 1.00 | 33.49 | O |
| ATOM | 2752 | O | HOH | W5032 | 25.981 | 5.485 | 38.408 | 1.00 | 30.01 | O |
| ATOM | 2753 | O | HOH | W5033 | 18.351 | −22.326 | 27.170 | 1.00 | 39.62 | O |
| ATOM | 2754 | O | HOH | W5034 | 27.692 | −22.175 | 28.942 | 1.00 | 33.59 | O |
| ATOM | 2755 | O | HOH | W5035 | 7.766 | 9.184 | 29.149 | 1.00 | 37.38 | O |
| ATOM | 2756 | O | HOH | W5036 | 27.081 | 7.365 | 5.947 | 1.00 | 37.77 | O |
| ATOM | 2757 | O | HOH | W5037 | 26.538 | 19.381 | 35.080 | 1.00 | 37.77 | O |
| ATOM | 2758 | O | HOH | W5038 | −4.201 | 17.799 | 6.607 | 1.00 | 32.96 | O |
| ATOM | 2759 | O | HOH | W5039 | 5.036 | 8.700 | 14.260 | 1.00 | 30.11 | O |
| ATOM | 2760 | O | HOH | W5040 | 0.851 | 9.642 | 42.538 | 1.00 | 22.98 | O |
| ATOM | 2761 | O | HOH | W5041 | 19.927 | 0.231 | 15.198 | 1.00 | 20.70 | O |
| ATOM | 2762 | O | HOH | W5042 | 38.613 | −9.206 | 13.685 | 1.00 | 46.18 | O |
| ATOM | 2763 | O | HOH | W5043 | 35.672 | −15.638 | 5.122 | 1.00 | 43.16 | O |
| ATOM | 2764 | O | HOH | W5044 | 14.774 | −8.966 | 39.562 | 1.00 | 27.43 | O |
| ATOM | 2765 | O | HOH | W5045 | −0.343 | −11.294 | 8.886 | 1.00 | 35.83 | O |
| ATOM | 2766 | O | HOH | W5046 | 0.384 | 7.133 | 40.132 | 1.00 | 36.11 | O |
| ATOM | 2767 | O | HOH | W5047 | −1.790 | −16.597 | 10.364 | 1.00 | 51.74 | O |
| ATOM | 2768 | O | HOH | W5048 | 17.913 | 8.434 | 22.684 | 1.00 | 19.14 | O |
| ATOM | 2769 | O | HOH | W5049 | 26.849 | 7.812 | 30.849 | 1.00 | 32.50 | O |
| ATOM | 2770 | O | HOH | W5050 | 31.682 | 10.495 | 7.280 | 1.00 | 43.96 | O |
| ATOM | 2771 | O | HOH | W5051 | 33.295 | 7.550 | 31.842 | 1.00 | 42.65 | O |
| ATOM | 2772 | O | HOH | W5052 | 26.726 | 22.667 | 9.308 | 1.00 | 42.17 | O |
| ATOM | 2773 | O | HOH | W5053 | 16.839 | 2.653 | 43.220 | 1.00 | 32.73 | O |
| ATOM | 2774 | O | HOH | W5054 | 28.300 | 3.705 | 0.074 | 1.00 | 31.84 | O |
| ATOM | 2775 | O | HOH | W5055 | 21.249 | 10.241 | 40.281 | 1.00 | 40.01 | O |
| ATOM | 2776 | O | HOH | W5056 | 18.910 | 0.164 | 43.189 | 1.00 | 35.91 | O |
| ATOM | 2777 | O | HOH | W5057 | 14.293 | 25.027 | 1.495 | 1.00 | 30.20 | O |
| ATOM | 2778 | O | HOH | W5058 | 15.432 | −0.416 | 17.518 | 1.00 | 26.65 | O |
| ATOM | 2779 | O | HOH | W5059 | 21.028 | 5.383 | 41.219 | 1.00 | 31.60 | O |
| ATOM | 2780 | O | HOH | W5060 | 27.025 | 7.703 | 33.714 | 1.00 | 38.38 | O |
| ATOM | 2781 | O | HOH | W5061 | 1.162 | −2.470 | 38.136 | 1.00 | 32.76 | O |
| ATOM | 2782 | O | HOH | W5062 | 7.118 | −4.468 | 3.111 | 1.00 | 25.42 | O |
| ATOM | 2783 | O | HOH | W5063 | 10.345 | 8.046 | 50.558 | 1.00 | 21.47 | O |
| ATOM | 2784 | O | HOH | W5064 | 1.601 | −6.957 | 7.392 | 1.00 | 19.01 | O |
| ATOM | 2785 | O | HOH | W5065 | 8.301 | 8.667 | 48.256 | 1.00 | 31.31 | O |
| ATOM | 2786 | O | HOH | W5066 | 13.857 | 7.152 | 1.988 | 1.00 | 29.65 | O |
| ATOM | 2787 | O | HOH | W5067 | −8.101 | −4.494 | 44.898 | 1.00 | 34.47 | O |
| ATOM | 2788 | O | HOH | W5068 | 31.896 | −17.405 | 6.589 | 1.00 | 41.64 | O |
| ATOM | 2789 | O | HOH | W5069 | 17.654 | −25.606 | 27.581 | 1.00 | 27.36 | O |
| ATOM | 2790 | O | HOH | W5070 | 23.222 | 14.650 | 26.416 | 1.00 | 26.05 | O |
| ATOM | 2791 | O | HOH | W5071 | 7.588 | −0.528 | 0.314 | 1.00 | 32.42 | O |
| ATOM | 2792 | O | HOH | W5072 | 37.956 | −17.275 | 4.444 | 1.00 | 27.98 | O |
| ATOM | 2793 | O | HOH | W5073 | 13.506 | −13.814 | 28.837 | 1.00 | 53.93 | O |
| ATOM | 2794 | O | HOH | W5074 | 32.339 | 6.763 | 6.234 | 1.00 | 44.01 | O |
| ATOM | 2795 | O | HOH | W5075 | 17.732 | −21.822 | 12.391 | 1.00 | 27.13 | O |
| ATOM | 2796 | O | HOH | W5076 | 2.962 | 14.623 | 32.943 | 1.00 | 35.34 | O |
| ATOM | 2797 | O | HOH | W5077 | 18.219 | −11.144 | 17.921 | 1.00 | 32.13 | O |
| ATOM | 2798 | O | HOH | W5078 | 21.073 | 24.057 | −0.096 | 1.00 | 34.29 | O |
| ATOM | 2799 | O | HOH | W5079 | 15.379 | −12.595 | 24.369 | 1.00 | 37.81 | O |
| ATOM | 2800 | O | HOH | W5080 | 26.131 | 21.190 | 0.182 | 1.00 | 31.04 | O |
| ATOM | 2801 | O | HOH | W5081 | −7.427 | −1.162 | 30.153 | 1.00 | 37.22 | O |
| ATOM | 2802 | O | HOH | W5082 | 9.376 | 19.648 | 13.353 | 1.00 | 46.89 | O |
| ATOM | 2803 | O | HOH | W5083 | 26.771 | −14.783 | 26.158 | 1.00 | 30.66 | O |
| ATOM | 2804 | O | HOH | W5085 | 24.158 | −4.023 | 38.598 | 1.00 | 29.95 | O |
| ATOM | 2805 | O | HOH | W5086 | 2.673 | −2.236 | 40.838 | 1.00 | 36.71 | O |
| ATOM | 2806 | O | HOH | W5087 | 14.901 | 15.719 | 31.793 | 1.00 | 66.05 | O |
| ATOM | 2807 | O | HOH | W5089 | 0.185 | 6.149 | 5.302 | 1.00 | 45.92 | O |
| ATOM | 2808 | O | HOH | W5090 | −3.050 | 13.963 | 3.439 | 1.00 | 31.57 | O |
| ATOM | 2809 | O | HOH | W5091 | 20.002 | 26.238 | 4.307 | 1.00 | 38.95 | O |
| ATOM | 2810 | O | HOH | W5092 | 9.528 | 7.010 | 25.049 | 1.00 | 40.00 | O |
| ATOM | 2811 | O | HOH | W5093 | 22.998 | 9.245 | 25.495 | 1.00 | 45.96 | O |
| ATOM | 2812 | O | HOH | W5094 | 25.633 | 5.504 | 41.203 | 1.00 | 33.74 | O |
| ATOM | 2813 | O | HOH | W5095 | 22.525 | 7.413 | 40.125 | 1.00 | 27.93 | O |
| ATOM | 2814 | O | HOH | W5096 | 25.772 | −0.812 | 38.637 | 1.00 | 43.16 | O |
| ATOM | 2815 | O | HOH | W5097 | 1.675 | 3.923 | 15.398 | 1.00 | 24.93 | O |
| ATOM | 2816 | O | HOH | W5098 | 3.426 | 25.166 | 2.688 | 1.00 | 33.60 | O |
| ATOM | 2817 | O | HOH | W5099 | 6.716 | 24.079 | 2.431 | 1.00 | 30.61 | O |
| ATOM | 2818 | O | HOH | W5100 | 13.776 | 2.826 | 15.316 | 1.00 | 35.09 | O |
| ATOM | 2819 | O | HOH | W5101 | 2.840 | 22.627 | 5.321 | 1.00 | 28.88 | O |
| ATOM | 2820 | O | HOH | W5102 | 4.306 | 26.695 | 0.803 | 1.00 | 45.75 | O |
| ATOM | 2821 | O | HOH | W5103 | 28.312 | 3.533 | 30.225 | 1.00 | 30.56 | O |
| ATOM | 2822 | O | HOH | W5104 | 7.296 | 5.105 | 45.493 | 1.00 | 44.55 | O |
| ATOM | 2823 | O | HOH | W5105 | 8.727 | −7.822 | 20.152 | 1.00 | 30.96 | O |
| ATOM | 2824 | O | HOH | W5106 | 15.910 | 2.991 | 1.288 | 1.00 | 26.19 | O |
| ATOM | 2825 | O | HOH | W5107 | 24.692 | 15.648 | 32.485 | 1.00 | 45.46 | O |
| ATOM | 2826 | O | HOH | W5108 | 10.229 | 10.752 | 16.032 | 1.00 | 39.52 | O |
| ATOM | 2827 | O | HOH | W5109 | 30.124 | 8.492 | 1.194 | 1.00 | 42.53 | O |
| ATOM | 2828 | O | HOH | W5110 | 38.431 | 9.460 | 12.406 | 1.00 | 34.79 | O |
| ATOM | 2829 | O | HOH | W5111 | 15.296 | −0.704 | 44.230 | 1.00 | 30.31 | O |
| ATOM | 2830 | O | HOH | W5112 | 22.219 | −25.058 | 0.518 | 1.00 | 40.16 | O |
| ATOM | 2831 | O | HOH | W5113 | 16.280 | −1.757 | 13.031 | 1.00 | 32.98 | O |
| ATOM | 2832 | O | HOH | W5114 | 7.672 | 5.118 | 50.500 | 1.00 | 42.44 | O |
| ATOM | 2833 | O | HOH | W5116 | 19.588 | 26.165 | 1.692 | 1.00 | 35.11 | O |
| ATOM | 2834 | O | HOH | W5117 | 29.689 | 0.468 | 33.170 | 1.00 | 50.63 | O |
| ATOM | 2835 | O | HOH | W5118 | 27.638 | 14.621 | 27.167 | 1.00 | 37.33 | O |
| ATOM | 2836 | O | HOH | W5119 | 25.674 | 24.122 | 6.832 | 1.00 | 44.26 | O |
| ATOM | 2837 | O | HOH | W5120 | 40.801 | −13.288 | 5.322 | 1.00 | 31.98 | O |
| ATOM | 2838 | O | HOH | W5121 | 36.143 | −1.829 | 16.148 | 1.00 | 41.24 | O |
| ATOM | 2839 | O | HOH | W5122 | 3.151 | 5.682 | 48.883 | 1.00 | 34.36 | O |
| ATOM | 2840 | O | HOH | W5123 | 32.693 | 14.445 | 9.290 | 1.00 | 34.74 | O |
| ATOM | 2841 | O | HOH | W5124 | 28.701 | 6.185 | 34.708 | 1.00 | 49.77 | O |
| ATOM | 2842 | O | HOH | W5125 | 26.196 | 1.517 | −1.280 | 1.00 | 39.43 | O |
| ATOM | 2843 | O | HOH | W5126 | −0.033 | 0.726 | 10.398 | 1.00 | 47.62 | O |
| ATOM | 2844 | O | HOH | W5127 | 21.383 | −3.597 | −1.922 | 1.00 | 51.56 | O |
| ATOM | 2845 | O | HOH | W5128 | 13.364 | 12.833 | 27.633 | 1.00 | 52.29 | O |
| ATOM | 2846 | O | HOH | W5129 | 21.714 | 12.615 | 35.721 | 1.00 | 33.09 | O |
| ATOM | 2847 | O | HOH | W5130 | 8.469 | 6.895 | 52.440 | 1.00 | 45.76 | O |
| ATOM | 2848 | O | HOH | W5131 | 26.110 | 4.660 | 30.832 | 1.00 | 35.30 | O |
| ATOM | 2849 | O | HOH | W5132 | 19.463 | −23.363 | 14.165 | 1.00 | 35.76 | O |
| ATOM | 2850 | O | HOH | W5133 | 13.443 | 20.073 | −9.252 | 1.00 | 40.04 | O |
| ATOM | 2851 | O | HOH | W5134 | 9.131 | 8.743 | 16.901 | 1.00 | 44.35 | O |
| ATOM | 2852 | O | HOH | W5135 | 2.141 | 8.144 | 15.984 | 1.00 | 43.82 | O |
| ATOM | 2853 | O | HOH | W5136 | 8.533 | 27.824 | 6.026 | 1.00 | 33.71 | O |
| ATOM | 2854 | O | HOH | W5137 | 23.489 | 23.402 | 0.284 | 1.00 | 47.94 | O |
| ATOM | 2855 | O | HOH | W5138 | 19.996 | 15.564 | −7.451 | 1.00 | 42.23 | O |
| ATOM | 2856 | O | HOH | W5139 | 3.830 | 6.820 | −6.454 | 1.00 | 43.70 | O |
| ATOM | 2857 | O | HOH | W5140 | 15.282 | 15.611 | 18.402 | 1.00 | 41.11 | O |
| ATOM | 2858 | O | HOH | W5141 | 17.265 | 26.061 | 11.713 | 1.00 | 39.14 | O |
| ATOM | 2859 | O | HOH | W5142 | 23.754 | 1.893 | −2.493 | 1.00 | 45.81 | O |
| ATOM | 2860 | O | HOH | W5143 | 26.488 | 3.338 | 6.608 | 1.00 | 42.72 | O |
| ATOM | 2861 | O | HOH | W5144 | 6.963 | 4.729 | 1.634 | 1.00 | 33.11 | O |
| ATOM | 2862 | O | HOH | W5145 | −0.293 | 3.358 | 3.517 | 1.00 | 47.82 | O |
| ATOM | 2863 | O | HOH | W5146 | 13.304 | 0.010 | 14.071 | 1.00 | 44.49 | O |
| ATOM | 2864 | O | HOH | W5147 | 14.485 | −0.911 | 11.196 | 1.00 | 34.10 | O |
| ATOM | 2865 | O | HOH | W5148 | 38.684 | −3.391 | 16.424 | 1.00 | 42.58 | O |
| ATOM | 2866 | O | HOH | W5149 | −0.378 | −3.887 | 41.179 | 1.00 | 39.92 | O |
| ATOM | 2867 | O | HOH | W5150 | 14.999 | 10.060 | 38.087 | 1.00 | 56.40 | O |
| ATOM | 2868 | O | HOH | W5151 | 27.440 | −10.394 | 33.773 | 1.00 | 51.57 | O |
| ATOM | 2869 | O | HOH | W5152 | 9.035 | −7.425 | 0.106 | 1.00 | 46.37 | O |
| ATOM | 2870 | O | HOH | W5153 | 5.110 | −2.372 | 2.316 | 1.00 | 33.68 | O |
| ATOM | 2871 | O | HOH | W5154 | 29.018 | −14.387 | 13.400 | 1.00 | 40.14 | O |
| ATOM | 2872 | O | HOH | W5155 | 1.676 | −2.447 | 9.577 | 1.00 | 41.85 | O |
| ATOM | 2873 | O | HOH | W5156 | 20.185 | 11.538 | −6.635 | 1.00 | 37.49 | O |
| ATOM | 2874 | O | HOH | W5157 | 15.389 | −28.857 | 26.962 | 1.00 | 41.90 | O |
| ATOM | 2875 | O | HOH | W5158 | −6.106 | 19.784 | 6.635 | 1.00 | 36.72 | O |
| ATOM | 2876 | O | HOH | W5159 | 5.517 | 17.184 | 13.233 | 1.00 | 48.23 | O |
| ATOM | 2877 | O | HOH | W5160 | 5.076 | 15.020 | 11.985 | 1.00 | 45.45 | O |
| ATOM | 2878 | O | HOH | W5161 | 5.445 | −7.849 | 27.659 | 1.00 | 39.11 | O |
| ATOM | 2879 | O | HOH | W5162 | 20.980 | 18.101 | 37.922 | 1.00 | 29.42 | O |
| ATOM | 2880 | O | HOH | W5163 | 16.748 | −29.146 | 3.293 | 1.00 | 42.39 | O |
| ATOM | 2881 | O | HOH | W5164 | 30.770 | 4.871 | 31.188 | 1.00 | 43.36 | O |
| ATOM | 2882 | O | HOH | W5165 | 3.854 | −8.929 | 29.337 | 1.00 | 54.81 | O |
| ATOM | 2883 | O | HOH | W5166 | 10.593 | 19.605 | 16.042 | 1.00 | 47.04 | O |
| ATOM | 2884 | O | HOH | W5167 | 42.095 | 8.840 | 19.150 | 1.00 | 49.61 | O |
| ATOM | 2885 | O | HOH | W5168 | 9.957 | 11.390 | 13.550 | 1.00 | 42.37 | O |
| ATOM | 2886 | O | HOH | W5169 | −4.606 | 21.333 | 1.399 | 1.00 | 48.40 | O |
| ATOM | 2887 | O | HOH | W5170 | 11.695 | 29.342 | 6.480 | 1.00 | 58.15 | O |
| ATOM | 2888 | O | HOH | W5171 | 23.099 | 25.128 | 3.657 | 1.00 | 40.99 | O |
| ATOM | 2889 | O | HOH | W5172 | 23.505 | 24.766 | −3.702 | 1.00 | 44.31 | O |
| ATOM | 2890 | O | HOH | W5173 | 14.447 | 17.461 | 20.573 | 1.00 | 40.93 | O |
| ATOM | 2891 | O | HOH | W5174 | 11.974 | 23.606 | 15.698 | 1.00 | 50.59 | O |
| ATOM | 2892 | O | HOH | W5175 | 33.868 | 19.345 | 11.374 | 1.00 | 39.71 | O |
| ATOM | 2893 | O | HOH | W5176 | 8.306 | −9.142 | 31.222 | 1.00 | 42.33 | O |
| ATOM | 2894 | O | HOH | W5177 | 12.171 | 14.379 | 29.266 | 1.00 | 52.75 | O |
| ATOM | 2895 | O | HOH | W5178 | 29.974 | −2.958 | 37.157 | 1.00 | 38.64 | O |
| ATOM | 2896 | O | HOH | W5179 | 30.177 | 1.102 | 3.621 | 1.00 | 43.39 | O |
| ATOM | 2897 | O | HOH | W5180 | 13.634 | 0.062 | 1.980 | 1.00 | 43.54 | O |
| ATOM | 2898 | O | HOH | W5181 | 9.893 | 10.792 | 25.719 | 1.00 | 35.63 | O |
| ATOM | 2899 | O | HOH | W5182 | 15.133 | 16.851 | 24.775 | 1.00 | 34.15 | O |
| TER | 2900 | | HOH | W5182 | | | | | | |
| ATOM | 2901 | N1A | COE | E4002 | 19.788 | −13.382 | 22.863 | 1.00 | 37.66 | N |
| ATOM | 2902 | C2A | COE | E4002 | 19.629 | −12.509 | 21.832 | 1.00 | 37.59 | C |
| ATOM | 2903 | N3A | COE | E4002 | 18.590 | −12.603 | 20.980 | 1.00 | 37.39 | N |
| ATOM | 2904 | C4A | COE | E4002 | 17.686 | −13.627 | 21.196 | 1.00 | 37.85 | C |
| ATOM | 2905 | C5A | COE | E4002 | 17.805 | −14.571 | 22.262 | 1.00 | 38.04 | C |
| ATOM | 2906 | C6A | COE | E4002 | 18.900 | −14.393 | 23.079 | 1.00 | 38.11 | C |
| ATOM | 2907 | N6A | COE | E4002 | 19.128 | −15.221 | 24.120 | 1.00 | 38.32 | N |
| ATOM | 2908 | N7A | COE | E4002 | 16.749 | −15.454 | 22.198 | 1.00 | 38.10 | N |
| ATOM | 2909 | C8A | COE | E4002 | 15.977 | −15.090 | 21.125 | 1.00 | 38.02 | C |
| ATOM | 2910 | N9A | COE | E4002 | 16.529 | −13.990 | 20.508 | 1.00 | 38.30 | N |
| ATOM | 2911 | C71 | COE | E4002 | 15.927 | −13.354 | 19.309 | 1.00 | 38.56 | C |
| ATOM | 2912 | C72 | COE | E4002 | 15.686 | −14.244 | 18.059 | 1.00 | 38.64 | C |
| ATOM | 2913 | O72 | COE | E4002 | 16.358 | −13.703 | 16.916 | 1.00 | 37.72 | O |
| ATOM | 2914 | C73 | COE | E4002 | 14.168 | −14.225 | 17.883 | 1.00 | 39.06 | C |
| ATOM | 2915 | O73 | COE | E4002 | 13.797 | −14.451 | 16.455 | 1.00 | 38.84 | O |
| ATOM | 2916 | P73 | COE | E4002 | 14.041 | −15.822 | 15.721 | 1.00 | 39.24 | P |
| ATOM | 2917 | O7A | COE | E4002 | 13.165 | −15.826 | 14.433 | 1.00 | 39.04 | O |
| ATOM | 2918 | O8A | COE | E4002 | 15.540 | −15.939 | 15.325 | 1.00 | 39.12 | O |
| ATOM | 2919 | O9A | COE | E4002 | 13.637 | −17.009 | 16.646 | 1.00 | 39.35 | O |
| ATOM | 2920 | C74 | COE | E4002 | 13.826 | −12.831 | 18.392 | 1.00 | 39.77 | C |
| ATOM | 2921 | O74 | COE | E4002 | 14.690 | −12.631 | 19.516 | 1.00 | 39.23 | O |
| ATOM | 2922 | C75 | COE | E4002 | 12.345 | −12.657 | 18.817 | 1.00 | 40.70 | C |
| ATOM | 2923 | O75 | COE | E4002 | 12.121 | −11.261 | 19.226 | 1.00 | 42.75 | O |
| ATOM | 2924 | P1A | COE | E4002 | 10.787 | −10.752 | 19.901 | 1.00 | 43.64 | P |
| ATOM | 2925 | O3A | COE | E4002 | 9.754 | −11.915 | 20.051 | 1.00 | 43.83 | O |
| ATOM | 2926 | O4A | COE | E4002 | 10.208 | −9.595 | 19.036 | 1.00 | 44.05 | O |
| ATOM | 2927 | O5A | COE | E4002 | 11.200 | −10.230 | 21.306 | 1.00 | 43.87 | O |
| TER | 2928 | | COE | E4002 | | | | | | |
| END | | | | | | | | | | |

What is claimed is:

1. A composition, comprising a crystal of isolated Streptococcus pneumoniae acyl carrier protein synthase.

2. The composition of claim 1, wherein said crystal effectively diffracts X-rays, and permits the determination of the atomic coordinates of said acyl carrier protein synthase to a resolution of about 2.0 Å.

3. The composition of claim 1, wherein said Streptococcus pneumoniae acyl carrier protein synthase has an active site cavity comprising the 3′,5′-adenosine diphosphate binding site shown in FIG. 9.

4. The composition of claim 1, wherein said Streptococcus pneumoniae acyl carrier protein synthase is a homotrimer, wherein each protomer comprises the following structural motifs:
(a) a three-stranded anti-parallel β-sheet formed by strands β1, β5, and β4;
(b) a long α-helix that packs diagonally against said β-sheet, together with α-helices α1, α2, α3, and α4 of an anti-parallel four helical bundle; and
(c) a long, extended loop with a two-strand anti-parallel β-sheet comprising strands β2 and β3,
wherein said structural motifs (a), (b), and (c) are organized such that said long helix α4 runs through said homotrimer, and is surrounded by the remainder of said structural motifs, as shown in FIGS. 8(B) and 8(C).

5. The composition of claim 1, wherein said crystal belongs to orthorhombic space group P2₁2₁2₁, with unit cell dimensions of a=49.8 Å, b=59.6 Å, and c=114.7 Å, or monoclinic space group C2, with unit cell dimensions of a=120.2 Å, b=62.3 Å, c=51.7 Å, and β=98.7°.

6. The composition of claim 1, wherein said acyl carrier protein synthase comprises selenocysteine or selenomethionine.

7. The composition of claim 1, wherein said acyl carrier protein synthase comprises a heavy metal atom.

8. The composition of claim 1, further comprising a chemical compound complexed covalently or non-covalently with said crystal.

9. The composition of claim 8, wherein said chemical compound is 3′,5′-adenosine diphosphate.

10. The composition claim 9, wherein said crystal belongs to monoclinic space group C2, with unit cell dimensions of a=120.2 Å, b=62.3 Å, c=51.7 Å, and β=98.7°.

11. The composition of claim 1, wherein said Streptococcus pneumoniae acyl carrier protein synthase has the amino acid sequence shown in SEQ ID NO:1.

12. The composition of claim 1, wherein said crystal has the atomic coordinates shown in Table 3 or Table 4.

13. The composition of claim 9, wherein said crystal has the atomic coordinates shown in Table 5.

14. A composition, comprising a crystal of Streptococcus pneumoniae acyl carrier protein synthase having the amino acid sequence shown in SEQ ID NO: 1 wherein methionine is substituted with selenomethioinine,
wherein said acyl carrier protein synthase is a homotrimer, wherein each protomer comprises the following structural motifs:
(d) a three-stranded anti-parallel β-sheet formed by strands β1, β5, and β4;
(e) a long α-helix that packs diagonally against said β-sheet, together with α-helices α1, α2, α3, and α4 of an anti-parallel four helical bundle; and
(f) a long, extended loop with a two-strand anti-parallel β-sheet comprising strands β2 and β3,
wherein said structural motifs (a), (b), and (c) are organized such that said long helix α4 runs through said homotrimer, and is surrounded by the remainder of said structural motifs, as shown in FIGS. 8(B) and 8(C);
wherein when said acyl carrier protein synthase is in native form, said crystal belongs to orthorhombic space group P2₁2₁2₁, with unit cell dimensions of a=49.8 Å, b=59.6 Å, and c=114.7 Å, or monoclinic space group C2, with unit cell dimensions of a=120.2 Å, b=62.3 Å, c=51.7 Å, and β=98.7°, and wherein said crystal has the atomic coordinates shown in Table 3 or Table 4, respectively; and
wherein when said acyl carrier protein synthase is complexed with 3′,5′-adenosine diphosphate, said crystal belongs to monoclinic space group C2, with unit cell dimensions of a=120.2 Å, b=62.3 Å, c=51.7 Å, and β=98.7°, and wherein said crystal has the atomic coordinates shown in Table 5.

15. An enzyme active site crystal structure comprising the 3′,5′-adenosine diphosphate binding site shown in FIG. 9.

16. The enzyme active site crystal structure of claim 15, wherein said enzyme comprises isolated, properly folded Streptococcus pneumoniae acyl carrier protein synthase, or a fragment thereof comprising said active site.

17. A method of isolating Streptococcus pneumoniae acyl carrier protein synthase, comprising:
(a) growing said Streptococcus pneumoniae in a medium lacking methionine but containing L-selenomethionine;
(b) preparing a cell extract of said Streptococcus pneumoniae;
(c) centrifuging said cell extract to produce a supernatant fraction, and collecting said supernatant fraction;
(d) chromatographing said supernatant fraction on a cation exchange column in buffer containing dithiothreitol or β-mercaptoethanol, and collecting fractions containing said Streptococcus pneumoniae acyl carrier protein synthase;
(e) chromatographing said fractions of step (d) on a gel filtration column in buffer containing dithiothreitol or β-mercaptoethanol, and collecting fractions containing said Streptococcus pneumoniae acyl carrier protein synthase comprising L-selenomethionine.

18. The method of claim 17, further comprising chromatographing said fractions of step (e) on an anion exchange column in buffer containing dithiothreitol or β-mercaptoethanol, and collecting fractions containing said Streptococcus pneumoniae acyl carrier protein synthase.

19. The method of claim 18, wherein said Streptococcus pneumoniae acyl carrier protein synthase has the amino acid sequence shown in SEQ ID NO:1, wherein methionine is replaced with L-selenomethionine.

20. Isolated Streptococcus pneumoniae acyl carrier protein synthase produced by the method of claim 19.

21. A method of producing a crystal of Streptococcus pneumoniae acyl carrier protein synthase that diffracts X-rays, comprising:
(a) providing Streptococcus pneumoniae acyl carrier protein synthase isolated according to claim 19;
(b) concentrating said acyl carrier protein synthase to 8 mg/ml in a solution containing 10 mM MgCl₂, 14 mM KCl, and 20 mM Tris-HCl at pH 7. 1 to produce a concentrated protein solution;
(c) equilibrating a 4 μl drop of said acyl carrier protein synthase in a solution comprising a mixture of 1: 1, v/v, concentrated protein solution as in step (b)/reservoir solution over a 500 μL reservoir solution comprising 8-15% polyethyleneglycol 4000, 200 mM ammonium sulfate, and 100 mM citrate buffer at pH 4. 5; and
(d) growing a crystal of said acyl carrier protein synthase by vapor diffusion at 294K for at least 4 to 5 days.

22. The method of claim 21, further comprising determining a three-dimensional structure of said crystal.

23. The method of claim 21, wherein said crystal belongs to orthorhombic space group P2₁2₁2₁, having unit cell parameters a=49.8 Å, b=59.6 Å, c=114.7 Å, or monoclinic space group C2, having unit cell parameters a=120.2 Å, b=62.3 Å, c=51.7 Å, β=98.7°), comprises one homotrimeric molecule per asymmetric unit, and has the atomic coordinates shown in Table 3 or Table 4.

24. The method of claim 21, further comprising testing the ability of a compound to form a complex with an active site of said acyl carrier protein synthase by including said compound in said concentrated protein solution of step (b).

25. The method of claim 21, further comprising contacting said crystal of acyl carrier protein synthase and a solution comprising a compound of interest to form a mixture, incubating said mixture to permit said compound to diffuse into said crystal, and determining whether said compound forms a complex with said acyl carrier protein synthase.

26. The method of claim 25, wherein said compound is 3′,5′-adenosine diphosphate.

27. The method of claim 25, wherein when said compound forms a complex with said acyl carrier protein synthase, determining a three-dimensional structure of said acyl carrier protein synthaselcompound crystal complex.

28. The method of claim 26, wherein said crystal belongs to monoclinic space group C2, having unit cell parameters a=120.2 Å, b=62.3 Å, c=51.7 Å, β=98.7°, comprises one homotrimeric molecule per asymmetric unit, and has the atomic coordinates shown in Table 5.

29. A crystal of Streptococcus pneumoniae acyl carrier protein synthase produced by the method of claim 21.

30. A crystal of Streptococcus pneumoniae acyl carrier protein synthase produced by the method of claim 24.

31. A crystal of Streptococcus pneumoniae acyl carrier protein synthase produced by the method of claim 25.
